SEQUENCE LISTING (45 sequences)

SEQ ID NO: 1 - DNA, 63 bp, synthetic construct
  source 1..63: mol_type = other DNA
tgccgtgtaatagtgagatctggaggctcaatnnnnnnnnnntcgtgactgggaaaaccagac

SEQ ID NO: 2 - DNA, 63 bp, synthetic construct
  source 1..63: mol_type = other DNA
tgccgtgtaatagtgagatctggacgacctaannnnnnnnnntcgtgactgggaaaaccagac

SEQ ID NO: 3 - DNA, 63 bp, synthetic construct
  source 1..63: mol_type = other DNA
tgccgtgtaatagtgagatctggataacgtcgnnnnnnnnnntcgtgactgggaaaaccagac

SEQ ID NO: 4 - DNA, 63 bp, synthetic construct
  source 1..63: mol_type = other DNA
tgccgtgtaatagtgagatctggaaagcatcgnnnnnnnnnntcgtgactgggaaaaccagac

SEQ ID NO: 5 - DNA, 63 bp, synthetic construct
  source 1..63: mol_type = other DNA
tgccgtgtaatagtgagatctggagcaattccnnnnnnnnnntcgtgactgggaaaaccagac

SEQ ID NO: 6 - DNA, 63 bp, synthetic construct
  source 1..63: mol_type = other DNA
tgccgtgtaatagtgagatctggagctacaacnnnnnnnnnntcgtgactgggaaaaccagac

SEQ ID NO: 7 - DNA, 63 bp, synthetic construct
  source 1..63: mol_type = other DNA
tgccgtgtaatagtgagatctggataggagctnnnnnnnnnntcgtgactgggaaaaccagac

SEQ ID NO: 8 - DNA, 63 bp, synthetic construct
  source 1..63: mol_type = other DNA
tgccgtgtaatagtgagatctggatggatggtnnnnnnnnnntcgtgactgggaaaaccagac

SEQ ID NO: 9 - DNA, 63 bp, synthetic construct
  source 1..63: mol_type = other DNA
tgccgtgtaatagtgagatctggatactagcgnnnnnnnnnntcgtgactgggaaaaccagac

SEQ ID NO: 10 - DNA, 63 bp, synthetic construct
  source 1..63: mol_type = other DNA
tgccgtgtaatagtgagatctggacagtgcttnnnnnnnnnntcgtgactgggaaaaccagac

SEQ ID NO: 11 - DNA, 63 bp, synthetic construct
  source 1..63: mol_type = other DNA
tgccgtgtaatagtgagatctggagatcaaggnnnnnnnnnntcgtgactgggaaaaccagac

SEQ ID NO: 12 - RNA, 2077 nt, synthetic construct
  source 1..2077: mol_type = other RNA
gggaaataagagagaaaagaagagtaagaagaaatataagaccccggcgccgccaccatggaggacgccaagaacatcaagaagggccctgcgccattctaccccctggaagatggaactgccggcgaacagttgcacaaggccatgaagcgctacgccttggtgcccggtactatcgccttcaccgacgcccacatcgaagtggacataacctacgcggagtacttcgaaatgtccgtgagactggccgaggctatgaagagatacggactgaacaccaaccaccgcatcgtggtctgctcggagaacagcctgcagttcttcatgcctgtcctcggtgcattgttcatcggagtggcggtggctcctgcaaacgacatctacaacgaacgcgaactcctgaactccatgggcatcagccagccgactgtagtgttcgtgagcaaaaagggactccagaagatcctgaacgtgcagaagaagctgccgatcattcaaaagattatcatcatggactccaagaccgactaccagggcttccagtcgatgtataccttcgtgacctcccatcttccgccggggttcaatgagtacgatttcgtgcctgagtctttcgaccgggacaagactatcgccctcatcatgaatagctccggatccaccggtctgcccaagggagtggccctgccacatcggaccgcctgcgtgagattctcacacgcccgggatcccatcttcggaaaccagatcatccccgacaccgcgattctgtccgtggtgccgtttcaccacggattcgggatgttcaccactctgggttacctgatttgcggcttccgcgtggtgctgatgtaccggttcgaggaggaactgtttctgcggagcctccaggattacaagattcagagcgcccttctggtcccgactctcttctcgttctttgccaagtccacacttattgacaaatacgatctctccaacctccatgaaattgcctcaggaggggctccgctgagcaaggaggtcggagaggctgtggccaagcggttccacctacccggcattagacagggttacggcctgacggaaaccacctccgcgatcctgatcactccggagggcgatgacaagcctggagctgtggggaaggtcgttccattctttgaagccaaggtcgtcgacctggataccggcaaaaccctgggtgtcaaccagcgcggagagctgtgtgtgcgcggacctatgattatgtccggctacgtgaacaaccccgaggccactaacgccctcatcgacaaggacggctggctgcactccggggacatcgcctactgggacgaggacgagcacttcttcattgtggaccggctgaagtcccttatcaagtataagggctaccaagtcgcaccggctgaactcgaatcaatcttgctgcaacaccccaacatcttcgacgccggagtggcaggactgcctgatgacgacgcgggagaactcccagcagccgtggtggtgctggaacatggcaaaaccatgaccgagaaggaaatcgtggattacgtggcgtcgcaagtcaccactgccaagaagctgagggggggagtggtctttgtggatgaagtgccgaaggggctgacggggaagctggacgccaggaagattagggagatcctgattaaggccaagaagggcggcaaaattgccgtgtaatagtgagatctggaaacagctatgaccatggaggctcaatnnnnnnnnnngtcgtgactgggaaaaccagactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacatgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - RNA, 2077 nt, synthetic construct
  source 1..2077: mol_type = other RNA
gggaaataagagagaaaagaagagtaagaagaaatataagaccccggcgccgccaccatggaggacgccaagaacatcaagaagggccctgcgccattctaccccctggaagatggaactgccggcgaacagttgcacaaggccatgaagcgctacgccttggtgcccggtactatcgccttcaccgacgcccacatcgaagtggacataacctacgcggagtacttcgaaatgtccgtgagactggccgaggctatgaagagatacggactgaacaccaaccaccgcatcgtggtctgctcggagaacagcctgcagttcttcatgcctgtcctcggtgcattgttcatcggagtggcggtggctcctgcaaacgacatctacaacgaacgcgaactcctgaactccatgggcatcagccagccgactgtagtgttcgtgagcaaaaagggactccagaagatcctgaacgtgcagaagaagctgccgatcattcaaaagattatcatcatggactccaagaccgactaccagggcttccagtcgatgtataccttcgtgacctcccatcttccgccggggttcaatgagtacgatttcgtgcctgagtctttcgaccgggacaagactatcgccctcatcatgaatagctccggatccaccggtctgcccaagggagtggccctgccacatcggaccgcctgcgtgagattctcacacgcccgggatcccatcttcggaaaccagatcatccccgacaccgcgattctgtccgtggtgccgtttcaccacggattcgggatgttcaccactctgggttacctgatttgcggcttccgcgtggtgctgatgtaccggttcgaggaggaactgtttctgcggagcctccaggattacaagattcagagcgcccttctggtcccgactctcttctcgttctttgccaagtccacacttattgacaaatacgatctctccaacctccatgaaattgcctcaggaggggctccgctgagcaaggaggtcggagaggctgtggccaagcggttccacctacccggcattagacagggttacggcctgacggaaaccacctccgcgatcctgatcactccggagggcgatgacaagcctggagctgtggggaaggtcgttccattctttgaagccaaggtcgtcgacctggataccggcaaaaccctgggtgtcaaccagcgcggagagctgtgtgtgcgcggacctatgattatgtccggctacgtgaacaaccccgaggccactaacgccctcatcgacaaggacggctggctgcactccggggacatcgcctactgggacgaggacgagcacttcttcattgtggaccggctgaagtcccttatcaagtataagggctaccaagtcgcaccggctgaactcgaatcaatcttgctgcaacaccccaacatcttcgacgccggagtggcaggactgcctgatgacgacgcgggagaactcccagcagccgtggtggtgctggaacatggcaaaaccatgaccgagaaggaaatcgtggattacgtggcgtcgcaagtcaccactgccaagaagctgagggggggagtggtctttgtggatgaagtgccgaaggggctgacggggaagctggacgccaggaagattagggagatcctgattaaggccaagaagggcggcaaaattgccgtgtaatagtgagatctggaaacagctatgaccatggacgacctaannnnnnnnnngtcgtgactgggaaaaccagactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacatgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - RNA, 2077 nt, synthetic construct
  source 1..2077: mol_type = other RNA
gggaaataagagagaaaagaagagtaagaagaaatataagaccccggcgccgccaccatggaggacgccaagaacatcaagaagggccctgcgccattctaccccctggaagatggaactgccggcgaacagttgcacaaggccatgaagcgctacgccttggtgcccggtactatcgccttcaccgacgcccacatcgaagtggacataacctacgcggagtacttcgaaatgtccgtgagactggccgaggctatgaagagatacggactgaacaccaaccaccgcatcgtggtctgctcggagaacagcctgcagttcttcatgcctgtcctcggtgcattgttcatcggagtggcggtggctcctgcaaacgacatctacaacgaacgcgaactcctgaactccatgggcatcagccagccgactgtagtgttcgtgagcaaaaagggactccagaagatcctgaacgtgcagaagaagctgccgatcattcaaaagattatcatcatggactccaagaccgactaccagggcttccagtcgatgtataccttcgtgacctcccatcttccgccggggttcaatgagtacgatttcgtgcctgagtctttcgaccgggacaagactatcgccctcatcatgaatagctccggatccaccggtctgcccaagggagtggccctgccacatcggaccgcctgcgtgagattctcacacgcccgggatcccatcttcggaaaccagatcatccccgacaccgcgattctgtccgtggtgccgtttcaccacggattcgggatgttcaccactctgggttacctgatttgcggcttccgcgtggtgctgatgtaccggttcgaggaggaactgtttctgcggagcctccaggattacaagattcagagcgcccttctggtcccgactctcttctcgttctttgccaagtccacacttattgacaaatacgatctctccaacctccatgaaattgcctcaggaggggctccgctgagcaaggaggtcggagaggctgtggccaagcggttccacctacccggcattagacagggttacggcctgacggaaaccacctccgcgatcctgatcactccggagggcgatgacaagcctggagctgtggggaaggtcgttccattctttgaagccaaggtcgtcgacctggataccggcaaaaccctgggtgtcaaccagcgcggagagctgtgtgtgcgcggacctatgattatgtccggctacgtgaacaaccccgaggccactaacgccctcatcgacaaggacggctggctgcactccggggacatcgcctactgggacgaggacgagcacttcttcattgtggaccggctgaagtcccttatcaagtataagggctaccaagtcgcaccggctgaactcgaatcaatcttgctgcaacaccccaacatcttcgacgccggagtggcaggactgcctgatgacgacgcgggagaactcccagcagccgtggtggtgctggaacatggcaaaaccatgaccgagaaggaaatcgtggattacgtggcgtcgcaagtcaccactgccaagaagctgagggggggagtggtctttgtggatgaagtgccgaaggggctgacggggaagctggacgccaggaagattagggagatcctgattaaggccaagaagggcggcaaaattgccgtgtaatagtgagatctggaaacagctatgaccatggataacgtcgnnnnnnnnnngtcgtgactgggaaaaccagactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacatgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - RNA, 2077 nt, synthetic construct
  source 1..2077: mol_type = other RNA
gggaaataagagagaaaagaagagtaagaagaaatataagaccccggcgccgccaccatggaggacgccaagaacatcaagaagggccctgcgccattctaccccctggaagatggaactgccggcgaacagttgcacaaggccatgaagcgctacgccttggtgcccggtactatcgccttcaccgacgcccacatcgaagtggacataacctacgcggagtacttcgaaatgtccgtgagactggccgaggctatgaagagatacggactgaacaccaaccaccgcatcgtggtctgctcggagaacagcctgcagttcttcatgcctgtcctcggtgcattgttcatcggagtggcggtggctcctgcaaacgacatctacaacgaacgcgaactcctgaactccatgggcatcagccagccgactgtagtgttcgtgagcaaaaagggactccagaagatcctgaacgtgcagaagaagctgccgatcattcaaaagattatcatcatggactccaagaccgactaccagggcttccagtcgatgtataccttcgtgacctcccatcttccgccggggttcaatgagtacgatttcgtgcctgagtctttcgaccgggacaagactatcgccctcatcatgaatagctccggatccaccggtctgcccaagggagtggccctgccacatcggaccgcctgcgtgagattctcacacgcccgggatcccatcttcggaaaccagatcatccccgacaccgcgattctgtccgtggtgccgtttcaccacggattcgggatgttcaccactctgggttacctgatttgcggcttccgcgtggtgctgatgtaccggttcgaggaggaactgtttctgcggagcctccaggattacaagattcagagcgcccttctggtcccgactctcttctcgttctttgccaagtccacacttattgacaaatacgatctctccaacctccatgaaattgcctcaggaggggctccgctgagcaaggaggtcggagaggctgtggccaagcggttccacctacccggcattagacagggttacggcctgacggaaaccacctccgcgatcctgatcactccggagggcgatgacaagcctggagctgtggggaaggtcgttccattctttgaagccaaggtcgtcgacctggataccggcaaaaccctgggtgtcaaccagcgcggagagctgtgtgtgcgcggacctatgattatgtccggctacgtgaacaaccccgaggccactaacgccctcatcgacaaggacggctggctgcactccggggacatcgcctactgggacgaggacgagcacttcttcattgtggaccggctgaagtcccttatcaagtataagggctaccaagtcgcaccggctgaactcgaatcaatcttgctgcaacaccccaacatcttcgacgccggagtggcaggactgcctgatgacgacgcgggagaactcccagcagccgtggtggtgctggaacatggcaaaaccatgaccgagaaggaaatcgtggattacgtggcgtcgcaagtcaccactgccaagaagctgagggggggagtggtctttgtggatgaagtgccgaaggggctgacggggaagctggacgccaggaagattagggagatcctgattaaggccaagaagggcggcaaaattgccgtgtaatagtgagatctggaaacagctatgaccatggaaagcatcgnnnnnnnnnngtcgtgactgggaaaaccagactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacatgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - RNA, 2077 nt, synthetic construct
  source 1..2077: mol_type = other RNA
gggaaataagagagaaaagaagagtaagaagaaatataagaccccggcgccgccaccatggaggacgccaagaacatcaagaagggccctgcgccattctaccccctggaagatggaactgccggcgaacagttgcacaaggccatgaagcgctacgccttggtgcccggtactatcgccttcaccgacgcccacatcgaagtggacataacctacgcggagtacttcgaaatgtccgtgagactggccgaggctatgaagagatacggactgaacaccaaccaccgcatcgtggtctgctcggagaacagcctgcagttcttcatgcctgtcctcggtgcattgttcatcggagtggcggtggctcctgcaaacgacatctacaacgaacgcgaactcctgaactccatgggcatcagccagccgactgtagtgttcgtgagcaaaaagggactccagaagatcctgaacgtgcagaagaagctgccgatcattcaaaagattatcatcatggactccaagaccgactaccagggcttccagtcgatgtataccttcgtgacctcccatcttccgccggggttcaatgagtacgatttcgtgcctgagtctttcgaccgggacaagactatcgccctcatcatgaatagctccggatccaccggtctgcccaagggagtggccctgccacatcggaccgcctgcgtgagattctcacacgcccgggatcccatcttcggaaaccagatcatccccgacaccgcgattctgtccgtggtgccgtttcaccacggattcgggatgttcaccactctgggttacctgatttgcggcttccgcgtggtgctgatgtaccggttcgaggaggaactgtttctgcggagcctccaggattacaagattcagagcgcccttctggtcccgactctcttctcgttctttgccaagtccacacttattgacaaatacgatctctccaacctccatgaaattgcctcaggaggggctccgctgagcaaggaggtcggagaggctgtggccaagcggttccacctacccggcattagacagggttacggcctgacggaaaccacctccgcgatcctgatcactccggagggcgatgacaagcctggagctgtggggaaggtcgttccattctttgaagccaaggtcgtcgacctggataccggcaaaaccctgggtgtcaaccagcgcggagagctgtgtgtgcgcggacctatgattatgtccggctacgtgaacaaccccgaggccactaacgccctcatcgacaaggacggctggctgcactccggggacatcgcctactgggacgaggacgagcacttcttcattgtggaccggctgaagtcccttatcaagtataagggctaccaagtcgcaccggctgaactcgaatcaatcttgctgcaacaccccaacatcttcgacgccggagtggcaggactgcctgatgacgacgcgggagaactcccagcagccgtggtggtgctggaacatggcaaaaccatgaccgagaaggaaatcgtggattacgtggcgtcgcaagtcaccactgccaagaagctgagggggggagtggtctttgtggatgaagtgccgaaggggctgacggggaagctggacgccaggaagattagggagatcctgattaaggccaagaagggcggcaaaattgccgtgtaatagtgagatctggaaacagctatgaccatggagcaattccnnnnnnnnnngtcgtgactgggaaaaccagactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacatgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 2077 nt, synthetic construct
  source 1..2077: mol_type = other RNA
gggaaataagagagaaaagaagagtaagaagaaatataagaccccggcgccgccaccatggaggacgccaagaacatcaagaagggccctgcgccattctaccccctggaagatggaactgccggcgaacagttgcacaaggccatgaagcgctacgccttggtgcccggtactatcgccttcaccgacgcccacatcgaagtggacataacctacgcggagtacttcgaaatgtccgtgagactggccgaggctatgaagagatacggactgaacaccaaccaccgcatcgtggtctgctcggagaacagcctgcagttcttcatgcctgtcctcggtgcattgttcatcggagtggcggtggctcctgcaaacgacatctacaacgaacgcgaactcctgaactccatgggcatcagccagccgactgtagtgttcgtgagcaaaaagggactccagaagatcctgaacgtgcagaagaagctgccgatcattcaaaagattatcatcatggactccaagaccgactaccagggcttccagtcgatgtataccttcgtgacctcccatcttccgccggggttcaatgagtacgatttcgtgcctgagtctttcgaccgggacaagactatcgccctcatcatgaatagctccggatccaccggtctgcccaagggagtggccctgccacatcggaccgcctgcgtgagattctcacacgcccgggatcccatcttcggaaaccagatcatccccgacaccgcgattctgtccgtggtgccgtttcaccacggattcgggatgttcaccactctgggttacctgatttgcggcttccgcgtggtgctgatgtaccggttcgaggaggaactgtttctgcggagcctccaggattacaagattcagagcgcccttctggtcccgactctcttctcgttctttgccaagtccacacttattgacaaatacgatctctccaacctccatgaaattgcctcaggaggggctccgctgagcaaggaggtcggagaggctgtggccaagcggttccacctacccggcattagacagggttacggcctgacggaaaccacctccgcgatcctgatcactccggagggcgatgacaagcctggagctgtggggaaggtcgttccattctttgaagccaaggtcgtcgacctggataccggcaaaaccctgggtgtcaaccagcgcggagagctgtgtgtgcgcggacctatgattatgtccggctacgtgaacaaccccgaggccactaacgccctcatcgacaaggacggctggctgcactccggggacatcgcctactgggacgaggacgagcacttcttcattgtggaccggctgaagtcccttatcaagtataagggctaccaagtcgcaccggctgaactcgaatcaatcttgctgcaacaccccaacatcttcgacgccggagtggcaggactgcctgatgacgacgcgggagaactcccagcagccgtggtggtgctggaacatggcaaaaccatgaccgagaaggaaatcgtggattacgtggcgtcgcaagtcaccactgccaagaagctgagggggggagtggtctttgtggatgaagtgccgaaggggctgacggggaagctggacgccaggaagattagggagatcctgattaaggccaagaagggcggcaaaattgccgtgtaatagtgagatctggaaacagctatgaccatggagctacaacnnnnnnnnnngtcgtgactgggaaaaccagactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacatgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - RNA, 2077 nt, synthetic construct
  source 1..2077: mol_type = other RNA
gggaaataagagagaaaagaagagtaagaagaaatataagaccccggcgccgccaccatggaggacgccaagaacatcaagaagggccctgcgccattctaccccctggaagatggaactgccggcgaacagttgcacaaggccatgaagcgctacgccttggtgcccggtactatcgccttcaccgacgcccacatcgaagtggacataacctacgcggagtacttcgaaatgtccgtgagactggccgaggctatgaagagatacggactgaacaccaaccaccgcatcgtggtctgctcggagaacagcctgcagttcttcatgcctgtcctcggtgcattgttcatcggagtggcggtggctcctgcaaacgacatctacaacgaacgcgaactcctgaactccatgggcatcagccagccgactgtagtgttcgtgagcaaaaagggactccagaagatcctgaacgtgcagaagaagctgccgatcattcaaaagattatcatcatggactccaagaccgactaccagggcttccagtcgatgtataccttcgtgacctcccatcttccgccggggttcaatgagtacgatttcgtgcctgagtctttcgaccgggacaagactatcgccctcatcatgaatagctccggatccaccggtctgcccaagggagtggccctgccacatcggaccgcctgcgtgagattctcacacgcccgggatcccatcttcggaaaccagatcatccccgacaccgcgattctgtccgtggtgccgtttcaccacggattcgggatgttcaccactctgggttacctgatttgcggcttccgcgtggtgctgatgtaccggttcgaggaggaactgtttctgcggagcctccaggattacaagattcagagcgcccttctggtcccgactctcttctcgttctttgccaagtccacacttattgacaaatacgatctctccaacctccatgaaattgcctcaggaggggctccgctgagcaaggaggtcggagaggctgtggccaagcggttccacctacccggcattagacagggttacggcctgacggaaaccacctccgcgatcctgatcactccggagggcgatgacaagcctggagctgtggggaaggtcgttccattctttgaagccaaggtcgtcgacctggataccggcaaaaccctgggtgtcaaccagcgcggagagctgtgtgtgcgcggacctatgattatgtccggctacgtgaacaaccccgaggccactaacgccctcatcgacaaggacggctggctgcactccggggacatcgcctactgggacgaggacgagcacttcttcattgtggaccggctgaagtcccttatcaagtataagggctaccaagtcgcaccggctgaactcgaatcaatcttgctgcaacaccccaacatcttcgacgccggagtggcaggactgcctgatgacgacgcgggagaactcccagcagccgtggtggtgctggaacatggcaaaaccatgaccgagaaggaaatcgtggattacgtggcgtcgcaagtcaccactgccaagaagctgagggggggagtggtctttgtggatgaagtgccgaaggggctgacggggaagctggacgccaggaagattagggagatcctgattaaggccaagaagggcggcaaaattgccgtgtaatagtgagatctggaaacagctatgaccatggataggagctnnnnnnnnnngtcgtgactgggaaaaccagactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacatgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - RNA, 2077 nt, synthetic construct
  source 1..2077: mol_type = other RNA
gggaaataagagagaaaagaagagtaagaagaaatataagaccccggcgccgccaccatggaggacgccaagaacatcaagaagggccctgcgccattctaccccctggaagatggaactgccggcgaacagttgcacaaggccatgaagcgctacgccttggtgcccggtactatcgccttcaccgacgcccacatcgaagtggacataacctacgcggagtacttcgaaatgtccgtgagactggccgaggctatgaagagatacggactgaacaccaaccaccgcatcgtggtctgctcggagaacagcctgcagttcttcatgcctgtcctcggtgcattgttcatcggagtggcggtggctcctgcaaacgacatctacaacgaacgcgaactcctgaactccatgggcatcagccagccgactgtagtgttcgtgagcaaaaagggactccagaagatcctgaacgtgcagaagaagctgccgatcattcaaaagattatcatcatggactccaagaccgactaccagggcttccagtcgatgtataccttcgtgacctcccatcttccgccggggttcaatgagtacgatttcgtgcctgagtctttcgaccgggacaagactatcgccctcatcatgaatagctccggatccaccggtctgcccaagggagtggccctgccacatcggaccgcctgcgtgagattctcacacgcccgggatcccatcttcggaaaccagatcatccccgacaccgcgattctgtccgtggtgccgtttcaccacggattcgggatgttcaccactctgggttacctgatttgcggcttccgcgtggtgctgatgtaccggttcgaggaggaactgtttctgcggagcctccaggattacaagattcagagcgcccttctggtcccgactctcttctcgttctttgccaagtccacacttattgacaaatacgatctctccaacctccatgaaattgcctcaggaggggctccgctgagcaaggaggtcggagaggctgtggccaagcggttccacctacccggcattagacagggttacggcctgacggaaaccacctccgcgatcctgatcactccggagggcgatgacaagcctggagctgtggggaaggtcgttccattctttgaagccaaggtcgtcgacctggataccggcaaaaccctgggtgtcaaccagcgcggagagctgtgtgtgcgcggacctatgattatgtccggctacgtgaacaaccccgaggccactaacgccctcatcgacaaggacggctggctgcactccggggacatcgcctactgggacgaggacgagcacttcttcattgtggaccggctgaagtcccttatcaagtataagggctaccaagtcgcaccggctgaactcgaatcaatcttgctgcaacaccccaacatcttcgacgccggagtggcaggactgcctgatgacgacgcgggagaactcccagcagccgtggtggtgctggaacatggcaaaaccatgaccgagaaggaaatcgtggattacgtggcgtcgcaagtcaccactgccaagaagctgagggggggagtggtctttgtggatgaagtgccgaaggggctgacggggaagctggacgccaggaagattagggagatcctgattaaggccaagaagggcggcaaaattgccgtgtaatagtgagatctggaaacagctatgaccatggatggatggtnnnnnnnnnngtcgtgactgggaaaaccagactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacatgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 20 - RNA, 2077 nt, synthetic construct
  source 1..2077: mol_type = other RNA
gggaaataagagagaaaagaagagtaagaagaaatataagaccccggcgccgccaccatggaggacgccaagaacatcaagaagggccctgcgccattctaccccctggaagatggaactgccggcgaacagttgcacaaggccatgaagcgctacgccttggtgcccggtactatcgccttcaccgacgcccacatcgaagtggacataacctacgcggagtacttcgaaatgtccgtgagactggccgaggctatgaagagatacggactgaacaccaaccaccgcatcgtggtctgctcggagaacagcctgcagttcttcatgcctgtcctcggtgcattgttcatcggagtggcggtggctcctgcaaacgacatctacaacgaacgcgaactcctgaactccatgggcatcagccagccgactgtagtgttcgtgagcaaaaagggactccagaagatcctgaacgtgcagaagaagctgccgatcattcaaaagattatcatcatggactccaagaccgactaccagggcttccagtcgatgtataccttcgtgacctcccatcttccgccggggttcaatgagtacgatttcgtgcctgagtctttcgaccgggacaagactatcgccctcatcatgaatagctccggatccaccggtctgcccaagggagtggccctgccacatcggaccgcctgcgtgagattctcacacgcccgggatcccatcttcggaaaccagatcatccccgacaccgcgattctgtccgtggtgccgtttcaccacggattcgggatgttcaccactctgggttacctgatttgcggcttccgcgtggtgctgatgtaccggttcgaggaggaactgtttctgcggagcctccaggattacaagattcagagcgcccttctggtcccgactctcttctcgttctttgccaagtccacacttattgacaaatacgatctctccaacctccatgaaattgcctcaggaggggctccgctgagcaaggaggtcggagaggctgtggccaagcggttccacctacccggcattagacagggttacggcctgacggaaaccacctccgcgatcctgatcactccggagggcgatgacaagcctggagctgtggggaaggtcgttccattctttgaagccaaggtcgtcgacctggataccggcaaaaccctgggtgtcaaccagcgcggagagctgtgtgtgcgcggacctatgattatgtccggctacgtgaacaaccccgaggccactaacgccctcatcgacaaggacggctggctgcactccggggacatcgcctactgggacgaggacgagcacttcttcattgtggaccggctgaagtcccttatcaagtataagggctaccaagtcgcaccggctgaactcgaatcaatcttgctgcaacaccccaacatcttcgacgccggagtggcaggactgcctgatgacgacgcgggagaactcccagcagccgtggtggtgctggaacatggcaaaaccatgaccgagaaggaaatcgtggattacgtggcgtcgcaagtcaccactgccaagaagctgagggggggagtggtctttgtggatgaagtgccgaaggggctgacggggaagctggacgccaggaagattagggagatcctgattaaggccaagaagggcggcaaaattgccgtgtaatagtgagatctggaaacagctatgaccatggatactagcgnnnnnnnnnngtcgtgactgggaaaaccagactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacatgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 21 - RNA, 2077 nt, synthetic construct
  source 1..2077: mol_type = other RNA
gggaaataagagagaaaagaagagtaagaagaaatataagaccccggcgccgccaccatggaggacgccaagaacatcaagaagggccctgcgccattctaccccctggaagatggaactgccggcgaacagttgcacaaggccatgaagcgctacgccttggtgcccggtactatcgccttcaccgacgcccacatcgaagtggacataacctacgcggagtacttcgaaatgtccgtgagactggccgaggctatgaagagatacggactgaacaccaaccaccgcatcgtggtctgctcggagaacagcctgcagttcttcatgcctgtcctcggtgcattgttcatcggagtggcggtggctcctgcaaacgacatctacaacgaacgcgaactcctgaactccatgggcatcagccagccgactgtagtgttcgtgagcaaaaagggactccagaagatcctgaacgtgcagaagaagctgccgatcattcaaaagattatcatcatggactccaagaccgactaccagggcttccagtcgatgtataccttcgtgacctcccatcttccgccggggttcaatgagtacgatttcgtgcctgagtctttcgaccgggacaagactatcgccctcatcatgaatagctccggatccaccggtctgcccaagggagtggccctgccacatcggaccgcctgcgtgagattctcacacgcccgggatcccatcttcggaaaccagatcatccccgacaccgcgattctgtccgtggtgccgtttcaccacggattcgggatgttcaccactctgggttacctgatttgcggcttccgcgtggtgctgatgtaccggttcgaggaggaactgtttctgcggagcctccaggattacaagattcagagcgcccttctggtcccgactctcttctcgttctttgccaagtccacacttattgacaaatacgatctctccaacctccatgaaattgcctcaggaggggctccgctgagcaaggaggtcggagaggctgtggccaagcggttccacctacccggcattagacagggttacggcctgacggaaaccacctccgcgatcctgatcactccggagggcgatgacaagcctggagctgtggggaaggtcgttccattctttgaagccaaggtcgtcgacctggataccggcaaaaccctgggtgtcaaccagcgcggagagctgtgtgtgcgcggacctatgattatgtccggctacgtgaacaaccccgaggccactaacgccctcatcgacaaggacggctggctgcactccggggacatcgcctactgggacgaggacgagcacttcttcattgtggaccggctgaagtcccttatcaagtataagggctaccaagtcgcaccggctgaactcgaatcaatcttgctgcaacaccccaacatcttcgacgccggagtggcaggactgcctgatgacgacgcgggagaactcccagcagccgtggtggtgctggaacatggcaaaaccatgaccgagaaggaaatcgtggattacgtggcgtcgcaagtcaccactgccaagaagctgagggggggagtggtctttgtggatgaagtgccgaaggggctgacggggaagctggacgccaggaagattagggagatcctgattaaggccaagaagggcggcaaaattgccgtgtaatagtgagatctggaaacagctatgaccatggacagtgcttnnnnnnnnnngtcgtgactgggaaaaccagactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacatgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 22 - RNA, 2277 nt, synthetic construct
  source 1..2277: mol_type = other RNA
aaaatccgtggctcgcagcaaacaccaaacggctcaataaaacaaacacaaaccaacaaaaagtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgtcacgatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtaatagctaactagacaaaacaccaaacaaacaagctgcgagccacggactt

SEQ ID NO: 23 - RNA, 2277 nt, synthetic construct
  source 1..2277: mol_type = other RNA
aaaatccgtggctcgcagcaaacaccaaaccgacctaaaaaacaaacacaaaccaacaaaaagtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgtcacgatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtaatagctaactagacaaaacaccaaacaaacaagctgcgagccacggactt

SEQ ID NO: 24 - RNA, 2277 nt, synthetic construct
  source 1..2277: mol_type = other RNA
aaaatccgtggctcgcagcaaacaccaaactaacgtcgaaaacaaacacaaaccaacaaaaagtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgtcacgatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtaatagctaactagacaaaacaccaaacaaacaagctgcgagccacggactt

SEQ ID NO: 25 - RNA, 2277 nt, synthetic construct
  source 1..2277: mol_type = other RNA
aaaatccgtggctcgcagcaaacaccaaacaagcatcgaaaacaaacacaaaccaacaaaaagtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgtcacgatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtaatagctaactagacaaaacaccaaacaaacaagctgcgagccacggactt

SEQ ID NO: 26 - RNA, 2277 nt, synthetic construct
  source 1..2277: mol_type = other RNA
aaaatccgtggctcgcagcaaacaccaaacgcaattccaaaacaaacacaaaccaacaaaaagtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgtcacgatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtaatagctaactagacaaaacaccaaacaaacaagctgcgagccacggactt

SEQ ID NO: 27 - RNA, 2277 nt, synthetic construct
  source 1..2277: mol_type = other RNA
aaaatccgtggctcgcagcaaacaccaaacgctacaacaaaacaaacacaaaccaacaaaaagtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgtcacgatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtaatagctaactagacaaaacaccaaacaaacaagctgcgagccacggactt

SEQ ID NO: 28 - RNA, 2277 nt, synthetic construct
  source 1..2277: mol_type = other RNA
aaaatccgtggctcgcagcaaacaccaaactaggagctaaaacaaacacaaaccaacaaaaagtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgtcacgatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtaatagctaactagacaaaacaccaaacaaacaagctgcgagccacggactt

SEQ ID NO: 29 - RNA, 2277 nt, synthetic construct
  source 1..2277: mol_type = other RNA
aaaatccgtggctcgcagcaaacaccaaactggatggtaaaacaaacacaaaccaacaaaaagtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgtcacgatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtaatagctaactagacaaaacaccaaacaaacaagctgcgagccacggactt

SEQ ID NO: 30 - RNA, 2277 nt, synthetic construct
  source 1..2277: mol_type = other RNA
aaaatccgtggctcgcagcaaacaccaaactactagcgaaaacaaacacaaaccaacaaaaagtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgtcacgatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtaatagctaactagacaaaacaccaaacaaacaagctgcgagccacggactt

SEQ ID NO: 31 - RNA, 2277 nt, synthetic construct
  source 1..2277: mol_type = other RNA
aaaatccgtggctcgcagcaaacaccaaaccagtgcttaaaacaaacacaaaccaacaaaaagtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgtcacgatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtaatagctaactagacaaaacaccaaacaaacaagctgcgagccacggactt

SEQ ID NO: 32 - protein, 50 aa, synthetic construct
  SITE 1..50: note = This sequence may encompass 1-10 GGGGS repeats
GGGGSGGGGSGGGGSGGGGSGGGGSGGGGSGGGGSGGGGSGGGGSGGGGS

SEQ ID NO: 33 - protein, 25 aa, synthetic construct
GGGGSGGGGSGGGGSGGGGSGGGGS

SEQ ID NO: 34 - DNA, 44 bp, synthetic construct
  source 1..44: mol_type = other DNA
cgccagaattccaaaatttgcctgcattaccggtcgatgcaacg

SEQ ID NO: 35 - DNA, 42 bp, synthetic construct
  source 1..42: mol_type = other DNA
gaccgcgcgcctgcagatatagaagataatcgcgaacatctt

SEQ ID NO: 36 - DNA, 45 bp, synthetic construct
  source 1..45: mol_type = other DNA
ctgagcggccgcctaggcccatggcgaatttactgacggtaccag

SEQ ID NO: 37 - DNA, 44 bp, synthetic construct
  source 1..44: mol_type = other DNA
aattctggtaccgtcagtaaattcgccatggcctaggcggccgc

SEQ ID NO: 38 - DNA, 36 bp, synthetic construct
  source 1..36: mol_type = other DNA
gcgatctgcaggtaagtatcaaggttacaagacagg

SEQ ID NO: 39 - DNA, 35 bp, synthetic construct
  source 1..35: mol_type = other DNA
atatgcgcgcctgtggagagaaaggcaaagtggat

SEQ ID NO: 40 - DNA, 39 bp, synthetic construct
  source 1..39: mol_type = other DNA
caataacttcgtataatgtatactatacgaagttattcg

SEQ ID NO: 41 - DNA, 47 bp, synthetic construct
  source 1..47: mol_type = other DNA
aattcgaataacttcgtatagtatacattatacgaagttattggtac

SEQ ID NO: 42 - DNA, 41 bp, synthetic construct
  source 1..41: mol_type = other DNA
caataacttcgtataatgtatactatacgaagttatctaga

SEQ ID NO: 43 - DNA, 49 bp, synthetic construct
  source 1..49: mol_type = other DNA
ccggtctagataacttcgtatagtatacattatacgaagttattggtac

SEQ ID NO: 44 - DNA, 43 bp, synthetic construct
  source 1..43: mol_type = other DNA
ttaagaataacttcgtataatgtatactatacgaagttattcg

SEQ ID NO: 45 - DNA, 43 bp, synthetic construct
  source 1..43: mol_type = other DNA
aattcgaataacttcgtatagtatacattatacgaagttattc

(19) United States
(12) Patent Application Publication
(10) Pub. No.: US 2024/0200118 A1
(43) Pub. Date: Jun. 20, 2024

(54) COMPOSITIONS AND METHODS FOR OPTIMIZING TROPISM OF DELIVERY SYSTEMS FOR RNA

(71) Applicant: Renagade Therapeutics Management Inc., Cambridge, MA (US)

(72) Inventors: Inna SHCHERBAKOVA, Holliston, MA (US); Grace CHEN, Cambridge, MA (US); Abril Fleitas BEITANS, Boston, MA (US); Brian GOODMAN, Boston, MA (US); Ciaran LAWLOR, Milton, MA (US); Kevin Yingxin YANG, Newton, MA (US)

(21) Appl. No.: 18/443,407

(22) Filed: Feb. 16, 2024

Related U.S. Application Data
(63) Continuation of application No. PCT/US22/40462, filed on Aug. 16, 2022.
(60) Provisional application No. 63/233,504, filed on Aug. 16, 2021.
(60) Provisional application No. 63/233,507, filed on Aug. 16, 2021.

(51) Int. Cl. C12Q 1/02; A61K 47/10; A61K 47/54; A61K 48/00

(52) U.S. Cl. CPC C12Q 1/025; A61K 47/10; A61K 47/543; A61K 48/0025

(57) ABSTRACT

The present disclosure details various compositions and/or methods of optimizing delivery systems the localized delivery of nucleic acid sequences, polypeptides or peptides for use in therapeutics and/or diagnostics.

### 1. FIELD OF THE DISCLOSURE

The present disclosure relates to optimized systems for delivery of nucleic acid sequences, polypeptides or peptides and methods of optimizing the delivery systems for targeted delivery.

### 2. BACKGROUND

Proteins have been the standard for therapeutics but the use of nucleic acids as therapeutic modalities for a variety of diseases and therapeutic indications has gained in prominence over the past few years. Various companies have shown that nucleic acids (e.g., siRNA, mRNA, circular RNA, DNA, etc.) can be more effective when compared to protein based therapies, but there is a need for targeted delivery systems for both nucleic acid and protein therapeutics in order to ensure the therapeutic is localized to a targeted cell, tissue or organ.

Current delivery systems, including lipid based delivery systems such as lipid nanoparticles, focus on protecting the cargo being delivered, but do not focus on localized delivery of the cargo or delivery system. To address this need, the present disclosure provides a tropism discovery platform for screening and developing targeting systems for localized delivery of nucleic acid and protein therapeutics.

### 3. SUMMARY

The present disclosure provides:

In an aspect of the invention, provided herein is a library of polynucleotides, wherein each of the polynucleotides in said library comprises:
a) a payload sequence region, said payload sequence region comprising a coding nucleic acid sequence or non-coding nucleic acid sequence;
b) at least one flanking sequence region located upstream or downstream of the payload sequence region;
c) at least one unique identifier; and
d) optionally, at least one regulatory sequence region.

In an aspect, the polynucleotides are DNA.

In an aspect, the polynucleotides are RNA.

In an aspect, the RNA are circular RNA (oRNA).

In an aspect, the RNA are short interfering RNA (siRNA)

In an aspect, the oRNA inhibits or suppresses the expression of a target of interest in a cell.

In an aspect, the siRNA inhibits or suppresses the expression of a target of interest in a cell.

In an aspect, the inhibition or suppression is about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In an aspect, the polynucleotides are substantially circular.

In an aspect, the RNA are small RNA.

In an aspect, polynucleotide comprises an internal ribosome entry site (IRES) sequence that is operably linked to the payload sequence region.

In an aspect, the RNA are microRNA.

In an aspect, the IRES sequence comprises a sequence derived from picornavirus complementary DNA, encephalomyocarditis virus (EMCV) complementary DNA, poliovirus complementary DNA, or an Antennapedia gene from Drosophila melanogaster.

In an aspect, the RNA are transfer RNA.

In an aspect, the polynucleotide comprises a termination element, wherein the termination element comprises at least one stop codon.

In an aspect, the polynucleotide comprises a regulatory element.

In an aspect, the polynucleotide comprises at least one masking agent.

In an aspect, the substantially circular polynucleotide is produced using in vitro transcription.

In an aspect the polynucleotide is produced using in vitro transcription

In an aspect, the payload sequence region comprises a non-coding nucleic acid sequence.

In an aspect, the payload sequence region comprises a coding nucleic acid sequence.

In an aspect, the coding nucleic acid sequence encodes a protein of interest for treatment of muscular dystrophy.

In an aspect, the protein of interest is Dystrophin.

In an aspect, the coding nucleic acid sequence encodes a protein of interest for treatment of cardiovascular disease.

In an aspect, the protein of interest is SERCA2a, GATA4, Tbx5, Mef2C, Hand2, or Myocd.

In an aspect, the coding nucleic acid sequence encodes a protein of interest for treatment of neurodegenerative disease.

In an aspect, the protein of interest is NGF, BDNF, GDNF, or NT-3.

In an aspect, the coding nucleic acid sequence encodes a protein of interest for treatment of chronic pain.

In an aspect, the protein of interest is GlyRal.

In an aspect, the coding nucleic acid sequence encodes a protein of interest for treatment of a glutamate decarboxylase disease or disorder.

In an aspect, the protein of interest is GAD65 or GAD67.

In an aspect, the coding nucleic acid sequence encodes a protein of interest for treatment of lung disease.

In an aspect, the protein of interest is CFTR.

In an aspect, the coding nucleic acid sequence encodes a protein of interest for treatment of hemophilia.

In an aspect, the protein of interest is Factor VIII or Factor IX.

In an aspect, the coding nucleic acid sequence encodes a protein of interest for treatment of neoplasia.

In an aspect, the protein of interest is PTEN, ATM, ATR, EGFR, ERBB2, ERBB3, ERBB4, Notch1, Notch2, Notch3, Notch4, AKT, AKT2, AKT3, HIF, HI Fla, HIF3a, Met, HRG, Bcl2, PPARalpha, PPAR gamma, WT1 (Wilms Tumor), FGF Receptor Family member 1, FGF Receptor Family member 2, FGF Receptor Family member 3, FGF Receptor Family member 4, FGF Receptor Family member 5, CDKN2a, APC, RB (retinoblastoma), MEN1, VHL, BRCA1, BRCA2, AR (Androgen Receptor), TSG101, IGF, IGF Receptor, Igf1 variant 1, Igf1 variant 2, Igf1 variant 3, Igf1 variant 4, Igf2 variant 1, Igf2 variant 2, Igf2 variant 3, Igf1 Receptor, Igf2 Receptor, Bax, Bcl2, caspase 1, caspase 2, caspase 3, caspase 4, caspase 6, caspase 7, caspase 8, caspase 9, caspase 12, Kras, or Ape.

In an aspect, the coding nucleic acid sequence encodes a protein of interest for treatment of age-related macular degeneration.

In an aspect, the protein of interest is Aber, Ccl2, Cc2, cp (ceruloplasmin), Timp3, cathepsin D, or Vldlr.

In an aspect, the coding nucleic acid sequence encodes a protein of interest for treatment of schizophrenia.

In an aspect, the protein of interest is Neuregulin (Nrgl), Erb4, Complexin-1 (Cplx1), Tph1 Tryptophan hydroxylase, Tph2 Tryptophan hydroxylase 2, Neurexin 1, GSK3, GSK3a, GSK3b, 5-HIT (Slc6a4), COMT, DRD (Drdla), SLC6A3, DAOA, DTNBPI, or Dao (Daol).

In an aspect, the coding nucleic acid sequence encodes a protein of interest for treatment of trinucleotide repeat disorders.

In an aspect, the protein of interest is HTT, SBMA/SMAXI/AR, FXN/X25, ATX3, ATXN1 and ATXN2, DMPK, Atrophin-1 and Atnl, CBP, VLDLR, Atxn7, or Atxn10.

In an aspect, the coding nucleic acid sequence encodes a protein of interest for treatment of fragile X syndrome.

In an aspect, the protein of interest is FMR2, FXRI, FXR2, or mGLUR5.

In an aspect, the coding nucleic acid sequence encodes a protein of interest for treatment of secretase related disorders.

In an aspect, the protein of interest is APH-1 (alpha and beta), Presenilin (Psenl), nicastrin (Ncstn), PEN-2.

In an aspect, the coding nucleic acid sequence encodes a protein of interest for treatment of ALS.

In an aspect, the protein of interest it SOD1, ALS2, STEX, FUS, TARD BP, or VEGF (VEGF-a, VEGF-b, VEGF-c).

In an aspect, the coding nucleic acid sequence encodes a protein of interest for treatment of autism.

In an aspect, the protein of interest is Mecp2, BZRAP1, MDGA2, Sema5A, or Neurexin 1.

In an aspect, the coding nucleic acid sequence encodes a protein of interest for treatment of Alzheimer's disease.

In an aspect, the protein of interest is E1, CHIP, UCH, UBB, Tau, LRP, PICALM, Clusterin, PSi, SORL1, CR1, Vldlr, Ubal, Uba3, CHIP28 (Aqpl, Aquaporin 1), Uchll, Uchl3, APP).

In an aspect, the coding nucleic acid sequence encodes a protein of interest for treatment of inflammation.

In an aspect, the protein of interest is IL-10, IL-1 (IL-Ia, IL-Ib), IL-13, IL-17 (IL-17a (CTLA8), IL-17b, IL-17c, IL-17d, IL-171), 11-23, Cx3crl, ptpn22, TNFa, NOD2/CARD15 for IBD, IL-6, IL-12 (IL-12a, IL-12b), CTLA4, Cx3cll, JAK3, JAKL, DCLREIC, ARTEMIS, SCIDA, RAG1, RAG2, ADA, PTPRC, CD45, LCA, IL7R, CD3D, T3D, IL2RG, SCIDXI, SCIDX, or IMD4.

In an aspect, the coding nucleic acid sequence encodes a protein of interest for treatment of Parkinson's Disease.

In an aspect, the protein of interest is x-Synuclein, DJ-1, LRRK2, Parkin, PINK1.

In an aspect, the coding nucleic acid sequence encodes a protein of interest for treatment of blood and coagulation disorders.

In an aspect, the blood and coagulation disorder is anemia, bare lymphocyte syndrome, bleeding disorders, hemophagocytic lymphohistiocytosis disorders, hemophilia A, hemophilia B, hemorrhagic disorders, leukocyte deficiencies and disorders, sickle cell anemia, or thalassemia.

In an aspect, the protein of interest is CRAN1, CDA1, RPS19, DBA, PKLR, PK1, NT5C3, UMPH1, PSNI, RHAG, RH50A, NRAMP2, SPTB, ALAS2, ANH1, ASB, ABCB7, ABC7, ASAT, TAPBP, TPSN, TAP2, ABCB3, PSF2, RING11, MHC2TA, C2TA, RFX5, RFXAP, RFX5, TBXA2R, P2RX1, P2X1, HFi, CFH, HUS, MCFD2, FANCA, FAC A, FA1, FA, FA A, FAAP95, FAAP90, FLJ34064, FANCB, FANCC, FACC, BRCA2, FANCDI, FANCD2, FANCD, FACD, FAD, FANCE, FACE, FANCF, XRCC9, FANCG, BR1PI, BACH1, FANCJ, PHF9, FANCL, FANCM, KIAA1596, PRF1, HPLH2, UNC13D, MUNC13-4, HPLH3, HLH3, FHL3, F8, FSC, PI, ATT, F5, ITGB2, CD18, LCAMB, LAD, EIF2B1, EIF2BA, EIF2B2, EIF2B3, EIF2B5, LVWM, CACH, CLE, EIF2B4, HBB, HBA2, HBB, HBD, LCRB, HBA1.

In an aspect, the coding nucleic acid sequence encodes a protein of interest for treatment of B-cell non-Hodgkin lymphoma or leukemia.

In an aspect, the protein of interest is BCL7A, BCL7, ALI, TCL5, SCL, TAL2, FLT3, NBS1, NBS, ZNFN1AI, 1KI, LYF1, HOXD4, HOX4B, BCR, CML, PHL, ALL, ARNT, KRAS2, RASK2, GMPS, AFIO, ARHGEF12, LARG, KIAA0382, CALM, CLTH, CEBPA, CEBP, CHIC2, BTL, FLT3, KIT, PBT, LPP, NPMI, NUP214, D9S46E, CAN, CAIN, RUNXI, CBFA2, AML1, WHSClLI, NSD3, FLT3, AF1Q, NPMI, NUMA1, ZNF145, PLZF, PML, MYL, STAT5B, AF1Q, CALM, CLTH, ARL11, ARLTS1, P2RX7, P2X7, BCR, CML, PHL, ALL, GRAF, NF1, VRNF, WSS, NFNS, PTPNII, PTP2C, SHP2, NS1, BCL2, CCND1, PRAD1, BCL1, TCRA, GATA1, GF1, ERYF1, NFE1, ABLI, NQO1, DIA4, NMOR1, NUP214, D9S46E, CAN, CAIN.

In an aspect, the coding nucleic acid sequence encodes a protein of interest for treatment of immune related diseases and disorders.

In an aspect, the protein of interest is KIR3DL1, NKAT3, NKB1, AMB11, K1R3DS1, IFNG, CXCL12, TNFRSF6, APT1, FAS, CD95, ALPS1A, IL2RG, SCIDX1, SCIDX, IMD4, CCL5, SCYA5, D17S136E, TCP228, IL10, CSIF, CMKBR2, CCR2, CMKBR5, CCCKR5 (CCR5), CD3E, CD3G, AICDA, AID, HIGM2, TNFRSF5, CD40, UNG, DGU, HIGM4, TNFSFS, CD40LG, HIGM1, IGM, FOXP3, IPEX, AIID, XPID, PIDX, TNFRSF14B, or TACI.

In an aspect, the coding nucleic acid sequence encodes a protein of interest for treatment of metabolic, liver, kidney and protein diseases and disorders.

In an aspect, the protein of interest is TTR, PALB, APOA1, APP, AAA, CVAP, ADI, GSN, FGA, LYZ, TTR, PALB, KRT18, KRT8, CIRHIA, NAIC, TEX292, KIAA1988, CFTR, ABCC7, CF, MRP7, SLC2A2, GLUT2, G6PC, G6PT, G6PT1, GAA, LAMP2, LAMPB, AGL, GDE, GBE1, GYS2, PYGL, PFKM, TCF1, HNF1A, MODY3, SCOD1, SCOl, CTNNB1, PDGFRL, PDGRL, PRLTS, AX1NI, AXIN, CTNNB1, TP53, P53, LFS1, IGF2R, MPRI, MET, CASP8, MCH5, UMOD, HNFJ, FJHN, MCKD2, ADMCKD2, PAH, PKU1, QDPR, DHPR, PTS, FCYT, PKHD1, ARPKD, PKD1, PKD2, PKD4, PKDTS, PRKCSH, G19P1, PCLD, or SEC63.

In an aspect, the coding nucleic acid sequence encodes a protein of interest for treatment of muscular/skeletal diseases and disorders.

In an aspect, the protein of interest is DMD, BMD, MYF6, LMNA, LMN1, EMD2, FPLD, CMDIA, HGPS, LGMDIB, LMNA, LMNI, EMD2, FPLD, CMDIA, FSHMD1A, FSHD1A, FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDCIC, LGMD21, TTN, CMD1G, TMD, LGMD2J, POMT1, CAV3, LGMD1C, SEPN1, SELN, RSMD1, PLEC1, PLTN, EBS1, LRP5, BMND1, LRP7, LR3, OPPG, VBCH2, CLCN7, CLC7, OPTA2, OSTMI, GL, TCIRG1, TIRC7, OC116, OPTB1, VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, or SMARD1.

In an aspect, the coding nucleic acid sequence encodes a protein of interest for treatment of neurological and neuronal diseases and disorders.

In an aspect, the protein of interest is SOD1, ALS2, STEX, FUS, TARDBP, VEGF (VEGF-a, VEGF-b, VEGF-c), APP, AAA, CVAP, ADI, APOE, AD2, PSEN2, AD4, STM2, APBB2, FE65LI, NOS3, PLAU, URK, ACE, DCPI, ACEI, MPO, PACIPI, PAXIPIL, PTIP, A2M, BLMH, BMH, PSEN1, AD3, Mecp2, BZRAP1, MDGA2, Sema5A, Neurexin 1, GLOl, MECP2, RTT, PPMX, MRX16, MRX79, NLGN3, NLGN4, KIAA1260, AUTSX2, FMR2, FXR1, FXR2, mGLUR5, HD, IT15, PRNP, PRIP, JPH3, JP3, HDL2, TBP, SCA17, NR4A2, NURR1, NOT, TINUR, SNCAIP, TBP, SCA17, SNCA, NACP, PARK1, PARK4, DJI, PARK7, LRRK2, PARK8, PINK1, PARK6, UCHL1, PARK5, SNCA, NACP, PARK1, PARK4, PRKN, PARK2, PDJ, DBH, NDUFV2, MECP2, RTT, PPMX, MRX16, MRX79, CDKL5, STK9, MECP2, RTT, PPMX, MRX16, MRX79, x-Synuclein, DJ-1, Neuregulin-1 (Nrgl), Erb4, Complexin-1 (Cplx1), Tph1 Tryptophan hydroxylase, Tph2, Tryptophan hydroxylase 2, Neurexin 1, GSK3, GSK3a, GSK3b, 5-HTT (Slc6a4), CONT, DRD (Drdla), SLC6A, DAOA, DTNBP1, Dao (Daol), APH-1(alpha and beta), Presenilin (Psenl), Nicastrin, (Ncstn), PEN-2, Nosl, Parpl, Nat1, Nat2, HTT, SBMA/SMAX1/AR, FXN/X25, ATX3, TXN, ATXN2, DMPK, Atrophin-1, Atnl, CBP, VLDLR, Atxn7, or AtxnlO.

In an aspect, the coding nucleic acid sequence encodes a protein of interest for treatment ofocular diseases and disorders.

In an aspect, the protein of interest is Aber, Ccl2, Cc2, cp (ceruloplasmin), Timp3, cathepsin-D, Vldlr, Ccr2, CRYAA, CRYA1, CRYBB2, CRYB2, PITX3, BFSP2, CP49, CP47, CRYAA, CRYAI, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYAI, GJA8, CX50, CAEl, GJA3, CX46, CZP3, CAE3, CCM1, CAM, KRIT1, APOA1, TGFBI, CSD2, CDGG1, CSD, BIGH3, CDG2, TACSTD2, TROP2, MlSI, VSX1, RINX, PPCD, PPD, KTCN, COL8A2, FECD, PPCD2, PIP5K3, CFD, KERA, CNA2, MYOC, TIGR, GLCIA, JO AG, GPOA, OPTN, GLC1E, FIP2, HYPL, NRP, CYPIBI, GLC3A, OPAl, NTG, NPG, CYPIBI, GLC3A, CRB1, RP12, CRX, CORD2, CRD, RPGRIPI, LCA6, CORD9, RPE65, RP20, AIPL1, LCA4, GUCY2D, GUC2D, LCA1, CORD6, RDH12, LCA3, ELOVL4, ADMD, STGD2, STGD3, RDS, RP7, PRPH2, PRPH, AVMD, AOFMD, or VMD2.

In an aspect, the polynucleotide comprises at least one modification. For example, at least 20%, at least 30%, at least 40%, at least 50%, at least 60%, at least 70%, at least 80%, about 90%, or at least 100% of the bases of the polynucleotide may be modified. In polynucleotide modifications of this disclosure, a specific base may comprise at least one modification. For example, the base adenine can be modified in polynucleotides of this disclosure, e.g., at least 20%, at least 30%, at least 40%, at least 50%, at least 60%, at least 70%, at least 80%, at least 90%, or at least 100% of the adenine bases can be modified. For example, the base guanine can be modified in polynucleotides of this disclosure, e.g., at least 20%, at least 30%, at least 40%, at least 50%, at least 60%, at least 70%, at least 80%, at least 90%, or at least 100% of the guanine bases can be modified. For example, the base cytosine can be modified in polynucleotides of this disclosure, e.g., at least 20%, at least 30%, at least 40%, at least 50%, at least 60%, at least 70%, at least 80%, at least 90%, or at least 100% of the cytosine bases can be modified. For example, the base uracil can be modified in polynucleotides of this disclosure, e.g., at least 20%, at least 30%, at least 40%, at least 50%, at least 60%, at least 70%, at least 80%, at least 90%, or at least 100% of the uracil bases can be modified.

In an aspect, the at least one modification is pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, or N2,N2-dimethyl-6-thio-guanosine.

In an aspect, the at least one unique identifier is detectable by florescence.

In an aspect, the at least one unique identifier is fluorescent dye.

In an aspect, the fluorescent dye is fluorescein, TAMRA (carboxytetramethylrhodamine), Cy dyes, Texas red, HEX, JOE, Oregon green, rhodamine 6 G, coumarin, pyrene, or DiOC6 (3,3′-dihexyloxacarbocyanine iodide).

In an aspect, the at least one unique identifier is fluorescent protein.

In an aspect, the fluorescent protein is Green Fluorescent Protein (GFP), Yellow Fluorescent Protein (YFP), Red Fluorescent Protein (RFP), Sirius, excitable blue fluorescent protein (EBFP2), cyan fluorescent protein (CFP), Cerulean, excitable green fluorescent protein (EGFP), excitable yellow fluorescent protein (EYFP), mOrange, mCherry, mPlum, NIR, iRFP, EosFP, PamCherry, Dronpa, Dreiklang, asFP595, mMaple, mGeo, mEos2, Dendra2, psCFP2, and 2,3,5,6-tetracarbazole-4-cyano-pyridine (CPy).

In an aspect, the fluorescent protein is luciferase.

In an aspect, the luciferase is Renilla luciferase, Gaussia luciferase, Nanoluc luciferase, Firefly luciferase, or Click Beetle luciferase.

In an aspect, the at least one unique identifier is a fluorescent nanoparticle.

In an aspect, the fluorescent nanoparticle is carbon dots, graphene quantum dots, gold nanorods, polymer-based nanoparticles, aggregation-induced emission dots, Conjugated Polymer nanoparticles (CP-dots), Gold nanospheres, Gold nano shells, Gold nanocages, or AIE pheromone.

In an aspect, the at least one unique identifier is fluorescent lipid.

In an aspect, the fluorescent lipid is DiR, DiD, DiO, DiI, Bodipy, or FL-Sphingomyelin.

In an aspect, the at least one unique identifier is β-galactosidase (β-gal).

In an aspect, the at least one unique identifier is a quencher molecule.

In an aspect, the quencher molecule is dimethylaminophenylazobenzoic acid (DABCYL), QSY 7, Cu(II) ion, Dabcyl, QSY 35, BHQ-0, Eclipse, BHQ-1, QSY 9, BHQ-2, ElleQuencher, Iowa Black, QSY 21, and BHQ-3.

In an aspect, the at least one unique identifier is a fluorophore.

In an aspect, the fluorophore is a quantum dot.

In an aspect, the quantum dot is CdSe/ZnS, CdTe/ZnS, CdTe/CdSe, CdSe/ZnTe, CdSe/CdTe/ZnSe, nAs/ZnSe, InAs/CdSe, InAs/InP, Cu:InP/ZnSe, InAsxP1-x/InP/ZnSe, CdS/CdSe, ZnSe/CdSe, ZnSe/InP/ZnS, ZnSe/InP/ZnS, CdTe/ZnSe, QD585, and QD655.

In an aspect, the fluorophore is an organic small molecule.

In an aspect, the organic small molecule is 7-dialkyl-amino-4-trifluoromethyl coumarin, rhodamine B, Coumarin 314, Lucifer Yellow CH, florescein, rhodamine 123, BODIPY FL NHS ester, Cy5, Rhodamine 6G, Silicon-rhodamine (SiR), Cy3, Cy5.5, Cy7, Cy2, ATT0655, ATTO680, ATTO700, Nitrobenzoxadiazole (NBD), 1,6-diphenyl-1,3,5-hexatriene (DPH), ABBERIOR™ ALEXA FLUOR™, ATTO™, DYLIGHT FLUOR™, ALEXA FLUOR 647™, and TOPFLUOR™.

In an aspect, the at least one unique identifier is a radioactive phosphate.

In an aspect, the at least one unique identifier is biotin.

In an aspect, the at least one unique identifier is digoxygenin.

In an aspect, the at least one unique identifier is dinitrophenyl (DNP).

In an aspect, the at least one unique identifier is Fluorescein.

In an aspect, the at least one unique identifier is fucose.

In an aspect, the at least one unique identifier is amine.

In an aspect, the at least one unique identifier is Texas Red®.

In an aspect, there are at least two unique identifiers.

In an aspect, there are 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more unique identifiers, e.g., from 2 to 50, from 2 to 100, from 2 to 100 unique identifiers, or from 2 to 5000 unique identifiers.

In an aspect, the unique identifiers are the same.

In an aspect, the unique identifiers different.

In an aspect, the length of the unique polynucleotide identifier is 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 nucleotides long.

In an aspect, the length of the unique peptide (e.g., polypeptide) identifier is 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 residues long.

In an aspect, a composition comprising a population of formulated polynucleotides, wherein each of the formulated polynucleotides is selected from the library of polynucleotides disclosed herein and wherein said polynucleotides are formulated in a delivery vehicle at a ratio of at least 1:1 (PN to DV), said delivery vehicle selected from group consisting of nanoparticles, lipid nanoparticles, micelles, exosomes, targeted lipid nanoparticles, non-lipid nanoparticles, liposomes, viral particles, and polymeric delivery particles.

In an aspect, the delivery vehicle comprises an exosome.

In an aspect, the delivery vehicle comprises a micelle.

In an aspect, the delivery vehicle comprises a non-lipid nanoparticle.

In an aspect, the delivery vehicle comprises a viral particle.

In an aspect, the viral particle is an AAV particle.

In an aspect, the delivery vehicle comprises a polymeric particle.

In an aspect, the delivery vehicle comprises a nanoparticle.

In an aspect, the nanoparticle comprises a lipid nanoparticle.

In an aspect, the population of formulated polynucleotides comprises at least two lipid nanoparticles.

In an aspect, the population of formulated polynucleotides comprises at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1,000, 1,025, 1,050, 1,075, 1,100, 1,125, 1,150, 1,175, 1,200, 1,225, 1,250, 1,275, 1,300, 1,325, 1,350, 1,375, 1,400, 1,425, 1,450, 1,475, 1,500, 1,525, 1,550, 1,575, 1,600, 1,625, 1,650, 1,675, 1,700, 1,725, 1,750, 1,775, 1,800, 1,825, 1,850, 1,875, 1,900, 1,925, 1,950, 1,975, 2,000, 2,025, 2,050, 2,075, 2,100, 2,125, 2,150, 2,175, 2,200, 2,225, 2,250, 2,275, 2,300, 2,325, 2,350, 2,375, 2,400, 2,425, 2,450, 2,475, 2,500, 2,525, 2,550, 2,575, 2,600, 2,625, 2,650, 2,675, 2,700, 2,725, 2,750, 2,775, 2,800, 2,825, 2,850, 2,875, 2,900, 2,925, 2,950, 2,975, 3,000, 3,025, 3,050, 3,075, 3,100, 3,125, 3,150, 3,175, 3,200, 3,225, 3,250, 3,275, 3,300, 3,325, 3,350, 3,375, 3,400, 3,425, 3,450, 3,475, 3,500, 3,525, 3,550, 3,575, 3,600, 3,625, 3,650, 3,675, 3,700, 3,725, 3,750, 3,775, 3,800, 3,825, 3,850, 3,875, 3,900, 3,925, 3,950, 3,975, 4,000, 4,025, 4,050, 4,075, 4,100, 4,125, 4,150, 4,175, 4,200, 4,225, 4,250, 4,275, 4,300, 4,325, 4,350, 4,375, 4,400, 4,425, 4,450, 4,475, 4,500, 4,525, 4,550, 4,575, 4,600, 4,625, 4,650, 4,675, 4,700, 4,725, 4,750, 4,775, 4,800, 4,825, 4,850, 4,875, 4,900, 4,925, 4,950, 4,975, 5,000, 5,025, 5,050, 5,075, 5,100, 5,125, 5,150, 5,175, 5,200, 5,225, 5,250, 5,275, 5,300, 5,325, 5,350, 5,375, 5,400, 5,425, 5,450, 5,475, 5,500, 5,525, 5,550, 5,575, 5,600, 5,625, 5,650, 5,675, 5,700, 5,725, 5,750, 5,775, 5,800, 5,825, 5,850, 5,875, 5,900, 5,925, 5,950, 5,975, 6,000, 6,025, 6,050, 6,075, 6,100, 6,125, 6,150, 6,175, 6,200, 6,225, 6,250, 6,275, 6,300, 6,325, 6,350, 6,375, 6,400, 6,425, 6,450, 6,475, 6,500, 6,525, 6,550, 6,575, 6,600, 6,625, 6,650, 6,675, 6,700, 6,725, 6,750, 6,775, 6,800, 6,825, 6,850, 6,875, 6,900, 6,925, 6,950, 6,975, 7,000, 7,025, 7,050, 7,075, 7,100, 7,125, 7,150, 7,175, 7,200, 7,225, 7,250, 7,275, 7,300, 7,325, 7,350, 7,375, 7,400, 7,425, 7,450, 7,475, 7,500, 7,525, 7,550, 7,575, 7,600, 7,625, 7,650, 7,675, 7,700, 7,725, 7,750, 7,775, 7,800, 7,825, 7,850, 7,875, 7,900, 7,925, 7,950, 7,975, 8,000, 8,025, 8,050, 8,075, 8,100, 8,125, 8,150, 8,175, 8,200, 8,225, 8,250, 8,275, 8,300, 8,325, 8,350, 8,375, 8,400, 8,425, 8,450, 8,475, 8,500, 8,525, 8,550, 8,575, 8,600, 8,625, 8,650, 8,675, 8,700, 8,725, 8,750, 8,775, 8,800, 8,825, 8,850, 8,875, 8,900, 8,925, 8,950, 8,975, 9,000, 9,025, 9,050, 9,075, 9,100, 9,125, 9,150, 9,175, 9,200, 9,225, 9,250, 9,275, 9,300, 9,325, 9,350, 9,375, 9,400, 9,425, 9,450, 9,475, 9,500, 9,525, 9,550, 9,575, 9,600, 9,625, 9,650, 9,675, 9,700, 9,725, 9,750, 9,775, 9,800, 9,825, 9,850, 9,875, 9,900, 9,925, 9,950, 9,975, or 10,000 individual lipid nanoparticles

In an aspect, the population of formulated polynucleotides comprises at least 2, 3, 4, 5, 6, 7, 8, 9, 10, 15, 20, 25, 30, 35, 40, 45, 50, 55, 60, 65, 70, 75, 80, 85, 90, 95, or 100 unique lipid nanoparticle varieties, each comprising a unique barcoded polynucleotide.

In an alternative aspect, the population of formulated polynucleotides comprises at least 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1,000, 1,025, 1,050, 1,075, 1,100, 1,125, 1,150, 1,175, 1,200, 1,225, 1,250, 1,275, 1,300, 1,325, 1,350, 1,375, 1,400, 1,425, 1,450, 1,475, 1,500, 1,525, 1,550, 1,575, 1,600, 1,625, 1,650, 1,675, 1,700, 1,725, 1,750, 1,775, 1,800, 1,825, 1,850, 1,875, 1,900, 1,925, 1,950, 1,975, 2,000, 2,025, 2,050, 2,075, 2,100, 2,125, 2,150, 2,175, 2,200, 2,225, 2,250, 2,275, 2,300, 2,325, 2,350, 2,375, 2,400, 2,425, 2,450, 2,475, 2,500, 2,525, 2,550, 2,575, 2,600, 2,625, 2,650, 2,675, 2,700, 2,725, 2,750, 2,775, 2,800, 2,825, 2,850, 2,875, 2,900, 2,925, 2,950, 2,975, 3,000, 3,025, 3,050, 3,075, 3,100, 3,125, 3,150, 3,175, 3,200, 3,225, 3,250, 3,275, 3,300, 3,325, 3,350, 3,375, 3,400, 3,425, 3,450, 3,475, 3,500, 3,525, 3,550, 3,575, 3,600, 3,625, 3,650, 3,675, 3,700, 3,725, 3,750, 3,775, 3,800, 3,825, 3,850, 3,875, 3,900, 3,925, 3,950, 3,975, 4,000, 4,025, 4,050, 4,075, 4,100, 4,125, 4,150, 4,175, 4,200, 4,225, 4,250, 4,275, 4,300, 4,325, 4,350, 4,375, 4,400, 4,425, 4,450, 4,475, 4,500, 4,525, 4,550, 4,575, 4,600, 4,625, 4,650, 4,675, 4,700, 4,725, 4,750, 4,775, 4,800, 4,825, 4,850, 4,875, 4,900, 4,925, 4,950, 4,975, 5,000, 5,025, 5,050, 5,075, 5,100, 5,125, 5,150, 5,175, 5,200, 5,225, 5,250, 5,275, 5,300, 5,325, 5,350, 5,375, 5,400, 5,425, 5,450, 5,475, 5,500, 5,525, 5,550, 5,575, 5,600, 5,625, 5,650, 5,675, 5,700, 5,725, 5,750, 5,775, 5,800, 5,825, 5,850, 5,875, 5,900, 5,925, 5,950, 5,975, 6,000, 6,025, 6,050, 6,075, 6,100, 6,125, 6,150, 6,175, 6,200, 6,225, 6,250, 6,275, 6,300, 6,325, 6,350, 6,375, 6,400, 6,425, 6,450, 6,475, 6,500, 6,525, 6,550, 6,575, 6,600, 6,625, 6,650, 6,675, 6,700, 6,725, 6,750, 6,775, 6,800, 6,825, 6,850, 6,875, 6,900, 6,925, 6,950, 6,975, 7,000, 7,025, 7,050, 7,075, 7,100, 7,125, 7,150, 7,175, 7,200, 7,225, 7,250, 7,275, 7,300, 7,325, 7,350, 7,375, 7,400, 7,425, 7,450, 7,475, 7,500, 7,525, 7,550, 7,575, 7,600, 7,625, 7,650, 7,675, 7,700, 7,725, 7,750, 7,775, 7,800, 7,825, 7,850, 7,875, 7,900, 7,925, 7,950, 7,975, 8,000, 8,025, 8,050, 8,075, 8,100, 8,125, 8,150, 8,175, 8,200, 8,225, 8,250, 8,275, 8,300, 8,325, 8,350, 8,375, 8,400, 8,425, 8,450, 8,475, 8,500, 8,525, 8,550, 8,575, 8,600, 8,625, 8,650, 8,675, 8,700, 8,725, 8,750, 8,775, 8,800, 8,825, 8,850, 8,875, 8,900, 8,925, 8,950, 8,975, 9,000, 9,025, 9,050, 9,075, 9,100, 9,125, 9,150, 9,175, 9,200, 9,225, 9,250, 9,275, 9,300, 9,325, 9,350, 9,375, 9,400, 9,425, 9,450, 9,475, 9,500, 9,525, 9,550, 9,575, 9,600, 9,625, 9,650, 9,675, 9,700, 9,725, 9,750, 9,775, 9,800, 9,825, 9,850, 9,875, 9,900, 9,925, 9,950, 9,975, or 10,000 unique lipid nanoparticle varieties, each comprising a unique barcoded polynucleotide.

In an aspect, the lipid nanoparticle comprises a targeted lipid nanoparticle.

In an aspect, the targeted lipid nanoparticle comprises one or more unique identifier.

In an aspect, at least one unique identifier is detectable by florescence.

In an aspect, the at least one unique identifier is fluorescent dye.

In an aspect, the fluorescent dye is fluorescein, TAMRA (carboxytetramethylrhodamine), Cy dyes, Texas red, HEX, JOE, Oregon green, rhodamine 6 G, coumarin, pyrene, or DiOC6 (3,3′-dihexyloxacarbocyanine iodide).

In an aspect, the at least one unique identifier is fluorescent protein.

In an aspect, the fluorescent protein is Green Fluorescent Protein (GFP), Yellow Fluorescent Protein (YFP), Red Fluorescent Protein (RFP), Sirius, excitable blue fluorescent protein (EBFP2), cyan fluorescent protein (CFP), Cerulean, excitable green fluorescent protein (EGFP), excitable yellow fluorescent protein (EYFP), mOrange, mCherry, mPlum, NIR, iRFP, EosFP, PamCherry, Dronpa, Dreiklang, asFP595, mMaple, mGeo, mEos2, Dendra2, psCFP2, and 2,3,5,6-tetracarbazole-4-cyano-pyridine (CPy).

In an aspect, the fluorescent protein is luciferase.

In an aspect, the luciferase is Renilla luciferase, Gaussia luciferase, Nanoluc luciferase, Firefly luciferase, or Click Beetle luciferase.

In an aspect, the at least one unique identifier is a fluorescent nanoparticle.

In an aspect, the fluorescent nanoparticle is carbon dots, graphene quantum dots, gold nanorods, polymer-based nanoparticles, aggregation-induced emission dots, Conjugated Polymer nanoparticles (CP-dots), Gold nanospheres, Gold nano shells, Gold nanocages, or AIE pheromone.

In an aspect, the at least one unique identifier is fluorescent lipid.

In an aspect, the fluorescent lipid is DiR, DiD, DiO, DiI, Bodipy, or FL-Sphingomyelin.

In an aspect, at least one unique identifier is β-galactosidase (β-gal).

In an aspect, at least one unique identifier is a quencher molecule.

In an aspect, the quencher molecule is dimethylaminophenylazobenzoic acid (DABCYL), QSY 7, Cu(II) ion, Dabcyl, QSY 35, BHQ-0, Eclipse, BHQ-1, QSY 9, BHQ-2, ElleQuencher, Iowa Black, QSY 21, and BHQ-3.

In an aspect, at least one unique identifier is a fluorophore.

In an aspect, the fluorophore is a quantum dot.

In an aspect, the quantum dot is CdSe/ZnS, CdTe/ZnS, CdTe/CdSe, CdSe/ZnTe, CdSe/CdTe/ZnSe, nAs/ZnSe, InAs/CdSe, InAs/InP, Cu:InP/ZnSe, InAsxP1-x/InP/ZnSe, CdS/CdSe, ZnSe/CdSe, ZnSe/InP/ZnS, ZnSe/InP/ZnS, CdTe/ZnSe, QD585, and QD655.

In an aspect, the fluorophore is an organic small molecule.

In an aspect, the organic small molecule is 7-dialkyl-amino-4-trifluoromethyl coumarin, rhodamine B, Coumarin 314, Lucifer Yellow CH, florescein, rhodamine 123, BODIPY FL NHS ester, Cy5, Rhodamine 6G, Silicon-rhodamine (SiR), Cy3, Cy5.5, Cy7, Cy2, ATT0655, ATTO680, ATTO700, Nitrobenzoxadiazole (NBD), 1,6-diphenyl-1,3,5-hexatriene (DPH), ABBERIOR™ ALEXA FLUOR™, ATTO™, DYLIGHT FLUOR™, ALEXA FLUOR 647™, and TOPFLUOR™.

In an aspect, at least one unique identifier is a radioactive phosphate.

In an aspect, at least one unique identifier is biotin.

In an aspect, at least one unique identifier is digoxygenin.

In an aspect, at least one unique identifier is dinitrophenyl (DNP).

In an aspect, at least one unique identifier is Fluorescein.

In an aspect, at least one unique identifier is fucose.

In an aspect, at least one unique identifier is amine.

In an aspect, the at least one unique identifier is Texas Red®.

In an aspect, the targeted lipid nanoparticle has attached, appended, bound or incorporated therein one or more targeting moieties selected from the group consisting of glycans, antibodies or fragments thereof, small molecules and peptides.

In an aspect, the targeted nanoparticle has attached, appended, bound or incorporated therein an antibody or fragment thereof.

In an aspect, the antibody or fragment thereof is a monospecific antibody, bispecific antibody, miniaturized antibody, diabody, unibody, intrabody, maxibody, Fab, Fab′, F(ab′)2, or Fv fragments of an antibody.

In an aspect, the target organ, tissue, or cell is not the liver.

In an aspect, the target organ, tissue, or cell is not the kidney.

In an aspect, the target organ, tissue, or cell is not the spleen.

In an aspect, a higher proportion of the polynucleotides are delivered to a target organ, tissue, or cell than to the liver following administration (e.g., intravenous administration) to a subject.

In an aspect, a higher proportion of the polynucleotides are delivered to a target organ, tissue, or cell than to the kidney following administration (e.g., intravenous administration) to a subject.

In an aspect, a higher proportion of the polynucleotides are delivered to a target organ, tissue, or cell than to the spleen following administration (e.g., intravenous administration) to a subject.

In an aspect, 1.5 times, 2 times, 3 times, 4 times, 5 times, 6 times, 7 times, 8 times, 9 times, 10 times, 20 times, 30 times, 40 times, 50 times, 60 times, 70 times, 80 times, 90 times, 100 times or more of the polynucleotides are delivered to a target organ, tissue, or cell than to the liver following administration (e.g., intravenous administration) to a subject.

In an aspect, 1.5 times, 2 times, 3 times, 4 times, 5 times, 6 times, 7 times, 8 times, 9 times, 10 times, 20 times, 30 times, 40 times, 50 times, 60 times, 70 times, 80 times, 90 times, 100 times or more of the polynucleotides are delivered to a target organ, tissue, or cell than to the kidney following administration (e.g., intravenous administration) to a subject.

In an aspect, 1.5 times, 2 times, 3 times, 4 times, 5 times, 6 times, 7 times, 8 times, 9 times, 10 times, 20 times, 30 times, 40 times, 50 times, 60 times, 70 times, 80 times, 90 times, 100 times or more of the polynucleotides are delivered to a target organ, tissue, or cell than to the spleen following administration (e.g., intravenous administration) to a subject.

In an aspect, provided herein is a method of validating/determining the cellular tropism of a candidate targeting system, comprising
a) contacting a non-human mammal with said candidate targeting system, said candidate targeting system being targeting systems of this disclosure, and
b) determining the cellular localization of the candidate targeting system by detecting for the presence of one or more of said unique identifiers in a plurality of tissues or organs.

In an aspect, the candidate targeting system comprises lipid nanoparticles.

In an aspect, unique identifier detected determine the localization of the polynucleotide.

In an aspect, unique identifier detected determine the localization of the formulation.

In an aspect, unique identifier detected determine the localization of the formulation polynucleotide.

In an aspect, the detection is by nuclear imaging.

In an aspect, the nuclear imaging is X-ray, magnetic resonance imaging (MRI), functional magnetic resonance imaging (fMRI), nuclear magnetic resonance imaging, computed tomography (CT), positron emission tomography (PET), single-photon emission computed tomography (SPECT), or absorption imaging.

In an aspect, the detection is by optical imaging.

In an aspect, the optical imaging is visible light microscopy, Raman spectroscopy, fluorescence microscopy, bioluminescence imaging (BLI), or optical coherence tomography.

In an aspect, the detection is by visible fluorescence microscopy.

In an aspect, the visible fluorescence microscopy is confocal fluorescence microscopy, fluorescence reflectance imaging, fluorescence molecular tomographic imaging, or Forster Resonance Energy Transfer (FRET).

In an aspect, the detection is by bioluminescence imaging (BLI).

In an aspect, the detection is by nucleotide sequencing.

In an aspect, the detection is by cell sorting techniques.

In an aspect, the cell sorting technique is magnetic beads, flow cytometry, cleavage of peptide with LC-MS/MS, or Fluorescence-activated Cell Sorting (FACS).

In an aspect, the method further comprises
a) obtaining one or more cells from the one or more organs or tissues of the subject, b) optionally amplifying one or more unique identifiers in the one or more cells, and c) identifying the one or more molecules associated with the unique identifiers in the one or more cells from the one or more organs or tissues of the subject, thereby identifying one or more targeted nanoparticles suitable for delivery to one or more tissues or organs of the subject.

In an aspect, tissues are adrenal medulla, adult fibrous tissue, blood vessels, bone, breast, bronchial lining, carotid body, cartilage, connective tissue, embryonic (myxomatous) fibrous tissue, epithelial, epithelium, fat, glandular epithelium (liver, kidney, bile duct), gonads, hematopoietic cells, lymph vessels, lymphoid tissue, meninges, mesothelium, muscle, nerve sheath, nervous, notochord, ovary, pancreas, parathyroid, pituitary, placenta, renal anlage, smooth muscle, stomach and intestines, stratified squamous, striated muscle, stroma, testis, thyroid, and transitional epithelium. As a non-limiting example, the tissue is connective tissue.

In an aspect, the organs are anal canal, arteries, ascending colon, bladder, bone marrow, brain, bronchi, bronchioles, bulbourethral glands, capillaries, cecum, cerebellum, cerebral hemispheres, cerebrum, cervix, choroid plexus, clitoris, cranial nerves, descending colon, diencephalon, duodenum, ear, enteric nervous system, epididymis, esophagus, external reproductive organs, fallopian tubes, gallbladder, ganglia, gustatory, gut-associated lymphoid tissue, heart, ileum, internal reproductive organs, interstitium, jejunum, joints, kidneys, large intestine, larynx, ligaments, liver, lungs, lymph node, lymphatic vessel, mammary glands, medulla oblongata, mesentery, midbrain, mouth, muscles of breathing, nasal cavity, nerves, olfactory, ovaries, pancreas, parotid glands, penis, pharynx, placenta, pons, prostate, rectum, salivary glands, scrotum, seminal vesicles, sigmoid colon, skeleton, skin, small intestine, spinal nerves, spleen, stomach, subcutaneous tissue, sublingual glands, submandibular glands, teeth, tendons, testes, the brainstem, the spinal cord, the ventricular system, thymus, tongue, tonsils, trachea, transverse colon, ureter, urethra, uterus, vagina, vas deferens, veins, or vulva.

In an aspect, provided herein is a method of delivering a therapeutic nucleic acid to a specific cellular location or site, comprising administering to a subject a therapeutic nucleic acid formulated in a delivery vehicle having validated tropism for said specific cellular location or site.

In an aspect, administration is enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, or spinal.

In an aspect, provided herein is method of identifying a treatment for a specific patient or patient population, the method comprising
a) obtaining a sample from the patient or members of the patient population, said sample containing one or more cells;
b) contacting said sample with one or more candidate targeting systems, said candidate targeting systems being targeting systems of this disclosure,
c) assaying for the presence of one or more of said unique identifiers, tags or labels in said one or more cells in said sample; and
d) identifying said unique identifiers, tags or labels in the one or more cells, thereby correlating the specific candidate targeting system with a cell-specific tropism indicator for the patient or patient population.

In an aspect, the sample is a tissue sample.

In an aspect, the sample is a tumor biopsy.

In an aspect, the sample is a blood sample.

This disclosure relates to methods for determining the biodistribution of one or more pharmaceutical delivery vehicles upon administration to a subject. The methods comprise formulating the pharmaceutical delivery vehicles to comprise a first engineered polynucleotide. The engineered polynucleotide comprises an optional payload sequence region comprising a coding nucleic acid sequence or non-coding nucleic acid sequence, a unique nucleotide identifier sequence, a flanking region sequence region, and optionally a regulatory sequence region. The flanking sequence region can be located upstream of the unique nucleotide identifier sequence and/or downstream of the unique nucleotide identifier sequence. The pharmaceutical delivery vehicles comprising the first engineered polynucleotide is administered to a subject. Next, a sample from the subject can be collected at one or more time points after administration of the one or more pharmaceutical delivery vehicles comprising the engineered polynucleotide. The at least one sample is then analyzed to determine the amount of the one or more pharmaceutical delivery vehicles present in the sample by measuring the amount of the unique identifier sequence present in the sample. When more than one distinct delivery vehicle is administered to the subject, each distinct pharmaceutical delivery vehicle comprises a distinct engineered polynucleotide comprising a different unique nucleotide identifier sequence.

The one or more pharmaceutical delivery vehicles can further comprise a second engineered polynucleotide.

This disclosure further relates to engineered polynucleotides. The engineered polynucleotide can comprise a payload sequence region comprising a coding nucleic acid sequence or non-coding nucleic acid sequence, a unique identifier sequence, a flanking sequence region, and optionally e a regulatory sequence region. The flanking region may be located upstream of the unique identifier sequence and/or downstream of the unique identifier sequence.

The engineered polynucleotides described herein can be DNA, RNA, circular RNA, mRNA, small RNA, miRNA, transfer RNA, siRNA, rRNA.

The engineered polynucleotides described herein can be RNA that inhibits or suppresses the expression of a target of interest in a cell. The inhibition or suppression by RNA can be about 30%, about 40%, about 50%, about 60%, about 70%, about 80%, about 85%, about 90%, about 95%, or about 100%, or at least about 20-30%, about 20-40%, about 20-50%, about 20-60%, about 20-70%, about 20-80%, about 20-90%, about 20-95%, about 20-100%, about 30-40%, about 30-50%, about 30-60%, about 30-70%, about 30-80%, about 30-90%, about 30-95%, about 30-100%, about 40-50%, about 40-60%, about 40-70%, about 40-80%, about 40-90%, about 40-95%, about 40-100%, about 50-60%, about 50-70%, about 50-80%, about 50-90%, about 50-95%, about 50-100%, about 60-70%, about 60-80%, about 60-90%, about 60-95%, about 60-100%, about 70-80%, about 70-90%, about 70-95%, about 70-100%, about 80-90%, about 80-95%, about 80-100%, about 90-95%, about 90-100% or about 95-100%.

The engineered polynucleotides can comprise an internal ribosome entry site (IRES) sequence that is operably linked to the payload sequence region. The engineered polynucleotides can comprise a promotor sequence that is operably linked to the payload sequence region. The engineered polynucleotides can comprise a termination element, wherein the termination element comprises at least one stop codon. The engineered polynucleotides can comprise a regulatory element. The engineered polynucleotides can comprise at least one masking agent. The engineered polynucleotides can be produced using in vitro transcription. The payload sequence regions n can comprise a non-coding nucleic acid sequence. The payload sequence regions can comprise a coding nucleic acid sequence. The engineered polynucleotides can comprise at least 5%, at least 10%, at least 20%, at least 30%, at least 40%, at least 50%, at least 60%, at least 70%, at least 80%, at least 90%, or at least 100% of the nucleotides as chemically modified nucleotides, wherein the chemically modified nucleotides are non-naturally occurring nucleotides.

This disclosure relates to a pharmaceutical composition comprising a pharmaceutical delivery vehicle (e.g. lipid nanoparticles) and an engineered polynucleotide as disclosed herein.

This disclosure also relates to a method of determining the biodistribution of one or more pharmaceutical delivery vehicles upon administration to a subject. The method of determining biodistribution comprises formulating the one or more pharmaceutical delivery vehicles to comprise a first engineered polynucleotide and a second engineered polynucleotide, administering the one or more pharmaceutical delivery vehicles comprising the first and second engineered polynucleotides to a subject, collecting a sample from the subject at one or more time points after administration of the one or more pharmaceutical delivery vehicles comprising the engineered polynucleotide, and analyzing the at least one sample to determine the amount of the one or more pharmaceutical delivery vehicles present in the sample by measuring the amount of unique identifier sequence(s) present in the sample. The first engineered polynucleotide can comprise the unique identifier sequence that is a barcode sequence, a flanking sequence region located at one or more locations selected from upstream of the unique identifier sequence and downstream of the unique identifier sequence, and optionally, a regulatory sequence region. The second engineered polynucleotides o can comprise a payload sequence region that has a coding nucleic acid sequence or a non-coding nucleic acid sequence. When more than one distinct pharmaceutical delivery vehicle is administered to the subject, each distinct pharmaceutical delivery vehicle comprises a distinct first engineered polynucleotide comprising a different unique identifier sequence.

Two or more distinct pharmaceutical delivery vehicles can be administered to the subject. At least five distinct pharmaceutical delivery vehicles can be administered to the subject. At least ten distinct pharmaceutical delivery vehicles can be administered to a subject.

The samples from the subject can be collected from tissue, organ or fluid selected from a group consisting of whole blood, plasma, lymph, anal canal, arteries, ascending colon, bladder, bone marrow, brain, bronchi, bronchioles, bulbourethral glands, capillaries, cecum, cerebellum, cerebral hemispheres, cerebrum, cervix, choroid plexus, clitoris, cranial nerves, descending colon, diencephalon, duodenum, ear, enteric nervous system, epididymis, esophagus, external reproductive organs, fallopian tubes, gallbladder, ganglia, gustatory, gut-associated lymphoid tissue, heart, ileum, internal reproductive organs, interstitium, jejunum, joints, kidneys, large intestine, larynx, ligaments, liver, lungs, lymph node, lymphatic vessel, mammary glands, medulla oblongata, mesentery, midbrain, mouth, muscles of breathing, nasal cavity, nerves, olfactory, ovaries, pancreas, parotid glands, penis, pharynx, placenta, pons, prostate, rectum, salivary glands, scrotum, seminal vesicles, sigmoid colon, skeleton, skin, small intestine, spinal nerves, spleen, stomach, subcutaneous tissue, sublingual glands, submandibular glands, teeth, tendons, testes, the brainstem, the spinal cord, the ventricular system, thymus, tongue, tonsils, trachea, transverse colon, ureter, urethra, uterus, vagina, vas deferens, veins, and vulva.

The pharmaceutical delivery vehicles can be administered intravenously, orally, or intramuscularly.

The first engineered polynucleotide can be RNA, RNA, mRNA, small RNA, miRNA, transfer RNA, siRNA, rRNA, or DNA.

The first engineered polynucleotide can comprise an internal ribosome entry site (IRES) sequence that is operably linked to the payload sequence region. The first engineered polynucleotide can comprise a promoter sequence that is operably linked to the payload sequence region The first engineered polynucleotide can comprise a termination element comprising at least one stop codon. The first engineered polynucleotide can comprise a regulatory element.

The first engineered polynucleotide can comprise at least one masking agent.

The first engineered polynucleotide can comprise the payload sequence.

The first engineered polynucleotide can comprise a non-coding nucleic acid sequence.

The first engineered polynucleotide can comprise a coding nucleic acid sequence.

The first engineered polynucleotide does not comprise the payload sequence.

acid sequence. The second engineered polynucleotide can comprise a payload sequence region comprising a coding nucleic acid sequence or non-coding nucleic acid sequence. The second engineered polynucleotide can be RNA, mRNA, RNA, small RNA, e miRNA, transfer RNA, siRNA, rRNA, or DNA.

The first or second engineered polynucleotides or engineered polypeptides can be chemically modified to comprise at least one non-naturally occurring nucleotide selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The unique identifier sequence (e.g., that is a barcode sequence) can comprise five to ten nucleotides.

The one or more pharmaceutical delivery vehicles can each independently be selected from lipid nanoparticles, non-lipid nanoparticles, exosomes, liposomes, micelles, viral particles, and polymeric nanoparticles. For example, the pharmaceutical delivery vehicles can be lipid nanoparticles. Exemplary lipid nanoparticles include, but are not limited to, ionizable lipids, structural lipids, c PEGylated lipids, and phospholipids.

Ionizable lipids can be selected from the group consisting of: the ionizable lipids disclosed in Table 2, and an ionizable lipid disclosed in one of: US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; and WO 2019/089828A1.

Structural lipids can be selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone.

PEGylated lipids may be selected from the group consisting of PEG-c-DOMG, PEG-DMG, PEG-DLPE, PEG-DMPE, PEG-DPPC, and PEG-DSPE.

Phospholipids can be selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

The lipid nanoparticles disclosed herein can comprise about 48.5 mol % of ionizable lipid, about 10 mol % of phospholipid, about 40 mol % of structural lipid, and about 1.5 mol % of PEGylated lipid. The lipid nanoparticles disclosed herein can comprise about 48.5 mol % of ionizable lipid, about 10 mol % of phospholipid, about 39 mol % of structural lipid, and about 2.5 mol % of PEGylated lipid.

The lipid nanoparticles disclosed herein can further comprise a targeting moiety operably connected to the lipid nanoparticle.

The lipid nanoparticles disclosed herein can further comprise one or more additional components selected from the group consisting of DDAB, EPC, 14PA, 18BMP, DODAP, DOTAP, and C12-200.

The DNA can comprise a polynucleotide selected from SEQ ID NOs. 1-11, or a sequence having at least about 70%, at least about 75%, about at least 80%, about at least 85%, about at least 90%, about at least 91%, about at least 92%, about at least 93%, about at least 94%, about at least 95%, about at least 96%, about at least 97%, about at least 98%, about at least 99%%, about at least 99.1%, about at least 99.2%, about at least 99.3%, about at least 99.4%, about at least 99.5%, about at least 99.6%, about at least 99.7%, about at least 99.8% or about at least 99.9% sequence identity with a sequence selected from SEQ ID NOs. 1-11.

### 4. BRIEF DESCRIPTION OF THE DRAWINGS

FIG. 1 is a diagram illustrating one embodiment of the tropism discovery platform of the present disclosure.

FIG. 2 is a diagram illustrating an originator polynucleotide construct of the present disclosure which may be linear or circular.

FIG. 3A is a diagram illustrating a series of benchmark polynucleotide constructs of the present disclosure which may include at least one barcode region (BC) and/or an inverted barcode region (CB) and a payload region (P).

FIG. 3B is a diagram illustrating a series of benchmark polynucleotide constructs of the present disclosure where the barcode region (BC) or inverted barcode region (CB) may overlap the payload region (P).

FIG. 3C is a diagram illustrating a series of benchmark polynucleotide constructs of the present disclosure which may include at least one tag and/or label.

FIG. 4A is a diagram illustrating a series of circular benchmark polynucleotide constructs of the present disclosure which may include at least one barcode region (BC) and/or an inverted barcode region (CB) and a payload region (P).

FIG. 4B is a diagram illustrating a series of circular benchmark polynucleotide constructs of the present disclosure where the barcode region (BC) or inverted barcode region (CB) may overlap the payload region (P).

FIG. 4C is a diagram illustrating a series of circular benchmark polynucleotide constructs of the present disclosure which may include at least one tag and/or label.

FIG. 5 is a diagram illustrating a series of delivery vehicles of the present disclosure.

FIGS. 6A and 6B are bar graphs showing the relative biodistribution of LNP compositions comprising barcoded DNA constructs DNA1 (F-1), DNA2 (F-3), and DNA3 (F-2), at 6 hours (FIG. 6A) and 24 hours (FIG. 6B) post dosing with a combined nine LNP formulation in BALB/C mice.

FIGS. 7A and 7B are bar graphs showing the relative biodistribution of LNP compositions comprising barcoded oRNA constructs mRNA4 (F-4), mRNA5 (F-6), and mRNA6 (F-5), at 6 hours (FIG. 7A) and 24 hours (FIG. 7B) post dosing with a combined nine LNP formulation in BALB/C mice.

FIGS. 8A and 8B are bar graphs showing the relative biodistribution of LNP compositions comprising barcoded oRNA constructs oRNA8A (F-8), oRNA7A (F-10), and oRNA9A (F-16), at 6 hours (FIG. 8A) and 24 hours (FIG. 8B) post dosing with a combined nine LNP formulation in BALB/C mice.

### 5. DETAILED DESCRIPTION

### I. Introduction to Tropism Delivery Systems

Nucleic acid therapy has emerged as the dominant method of treating various diseases and therapeutic indications given the versatility, lower immune response and higher potency as compared to traditional therapies. For example, nucleic acid therapy includes the use of small interfering (siRNA) to reduce the translation of messenger RNA (mRNA), mRNA as a way to produce a target of interest, circular RNA (oRNA) which can provide continuous production of a polypeptide or peptide or can be a sponge to compete with other RNA molecules, and viral vectors to provide a continuous production of a target of interest. However, some nucleic acids are unstable and easily degraded so they need to be formulated to prevent the degradation and to aid in the intracellular delivery of the nucleic acids.

Current pharmaceutical delivery vehicles, including lipid based pharmaceutical delivery vehicles such as lipid nanoparticles and liposomes, focus on protecting the cargo but do not concentrate on localizing the delivery of the cargo or pharmaceutical delivery vehicle to a specific area in vivo.

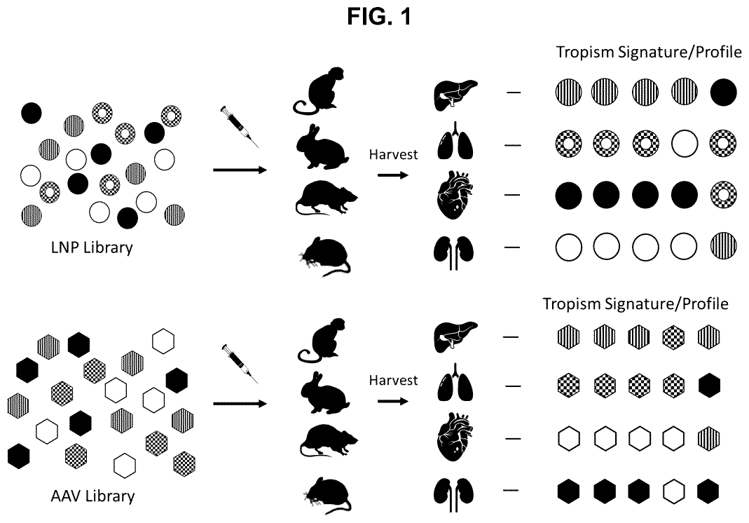

Provided herein is a tropism discovery platform for evaluating targeting systems for localized delivery to a specific target area, cell or tissue. Also provided are materials and methods for evaluating polynucleotides for the therapeutic treatment of a disease or disorder in a subject in need thereof. As shown in FIG. 1, the tropism discovery platform can be used to evaluate a lipid nanoparticle (LNP) library and/or a library of AAVs in order to determine the tropism or signature profile of the targeting systems in the library. The library can be administered to a subject (e.g., non-human primate, rabbit, mouse, rat or another mammal) and the organs and tissues of the subject are scanned and/or harvested and analyzed to determine the location of the identifiers (e.g., barcodes, labels, signals and/or tags) contained in or associated with the LNPs or the AAVs in the library. This analysis provides the tropism signature or profile of each LNP and AAV in the library.

a. Originator Construct Architecture

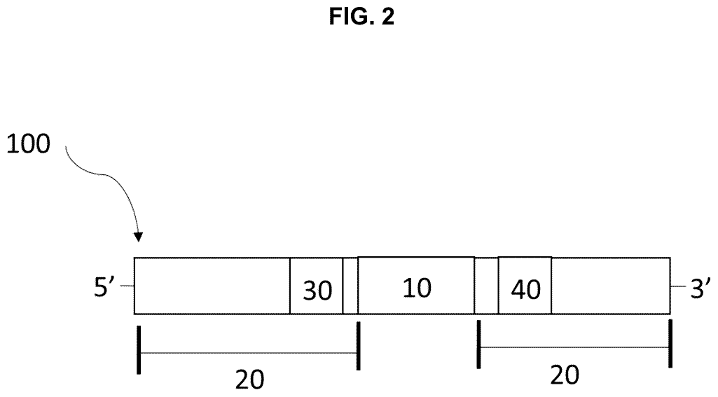

The targeting systems of the tropism discovery platform may include originator constructs (e.g., polynucleotides, polynucleotide plasmids) which encode or include a cargo or payload. An example of an originator polynucleotide construct 100, which may be linear or circular, is provided in FIG. 2. The originator polynucleotide construct 100 may include at least one payload region 10 which is or encodes a payload or cargo of interest. The originator polynucleotide construct 100 may contain 1 or 2 flanking regions 20 and the flanking regions 20 may be located 5′ to the payload region 10 or 3′ to the payload region 10. In some instances the originator polynucleotide construct 100 does not contain a flanking region 20. The flanking region 20 of the originator polynucleotide construct 100 may include at least one regulatory region 30. At least one flanking region 20 of the originator polynucleotide construct 100 may include at least one identifier region 40. The identifier region 40 may be, but is not limited to, a barcode, label, signal and/or tag. Additionally, the identifier region 40 may be located within the payload region 10 or may be located in the payload region 10 and at least one flanking region 20.

The originator polynucleotide constructs described herein can be of any suitable size (e.g., can contain any desirable number of nucleotides) and typically is of a size that is efficiently formulated (e.g., associated) with a pharmaceutical delivery vehicle. In some embodiments, the originator construct comprises from about 5 to about 10,000 nucleotides. As non-limiting examples, the length of the originator construct may be from 5 to 30, from 5 to 50, from 5 to 100, from 5 to 250, from 5 to 500, from 5 to 1,000, from 5 to 1,500, from 5 to 3,000 from 5 to 5,000, from 5 to 7,000, from 5 to 10,000 from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 100 to 250, from 100 to 500, from 100 to 1,000, from 100 to 1,500, from 100 to 3,000, from 100 to 5,000, from 100 to 7,000, from 100 to 10,000, from 500 to 1,000, from 500 to 1,500, from 500 to 2,000, from 500 to 3,000, from 500 to 5,000, from 500 to 7,000, from 500 to 10,000, from 1,000 to 1,500, from 1,000 to 2,000, from 1,000 to 3,000, from 1,000 to 5,000, from 1,000 to 7,000, from 1,000 to 10,000, from 1,500 to 3,000, from 1,500 to 5,000, from 1,500 to 7,000, from 1,500 to 10,000, from 2,000 to 3,000, from 2,000 to 5,000, from 2,000 to 7,000, from 2,000 to 10,000, from 3,000 to 5,000, from 3,000 to 7,000, from 3,000 to 10,000, from 5,000 to 7,000, from 5,000 to 10,000, and from 7,000 to 10,000.

In some embodiments, the length of the payload region is greater than about 5 nucleotides in length such as, but not limited to, at least or greater than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 4,000, 5,000, 6,000, 7,000, 8,000, 9,000, 10,000 or more than 10,000 nucleotides.

In some embodiments, the flanking region may range independently from 0 to 10,000 nucleotides in length such as, but not limited to, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 4,000, 5,000, 6,000, 7,000, 8,000, 9,000, and 10,000.

In some embodiments, the regulatory region may range independently from 0 to 3,000 nucleotides in length such as, but not limited to, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000.

In some embodiments, the originator construct may be cyclized, or concatemerized, to generate a molecule to assist interactions between 3′ and 5′ ends of the originator construct

Originator constructs which include at least one identifier (e.g., barcodes, labels, signals and/or tags) are referred to as benchmark constructs. In some embodiments, the benchmark construct is an engineered polynucleotide. The benchmark polynucleotide construct may comprise 1, 2, 3, 4, 5, 6, 7, 8, 9, or 10 or more identifiers which may be the same or different throughout the benchmark polynucleotide construct.

In some embodiments, the identifier region (also referred to as an identifier sequence, or a unique identifier sequence) may range independently from 1 to 3,000 nucleotides in length such as, but not limited to, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides. As a non-limiting example the identifier region may be 1-5 nucleotides, 2-5 nucleotides, 3-5 nucleotides, 2-7 nucleotides, 3-7 nucleotides, 1-10 nucleotides, 2-10 nucleotides, 3-10 nucleotides, 5-10 nucleotides, 7-10 nucleotides, 1-15 nucleotides, 2-15 nucleotides, 3-15 nucleotides, 5-15 nucleotides, 7-15 nucleotides, 10-15 nucleotides, 12-15 nucleotides, 1-20 nucleotides, 2-20 nucleotides, 3-20 nucleotides, 5-20 nucleotides, 7-20 nucleotides, 10-20 nucleotides, 12-20 nucleotides, 15-20 nucleotides, 17-20 nucleotides, 1-25 nucleotides, 2-25 nucleotides, 3-25 nucleotides, 5-25 nucleotides, 7-25 nucleotides, 10-25 nucleotides, 12-25 nucleotides, 15-25 nucleotides, 17-25 nucleotides, 20-25 nucleotides, 1-30 nucleotides, 2-30 nucleotides, 3-30 nucleotides, 5-30 nucleotides, 7-30 nucleotides, 10-30 nucleotides, 12-30 nucleotides, 15-30 nucleotides, 17-30 nucleotides, 20-30 nucleotides, 25-30 nucleotides, 1-35 nucleotides, 2-35 nucleotides, 3-35 nucleotides, 5-35 nucleotides, 7-35 nucleotides, 10-35 nucleotides, 12-35 nucleotides, 15-35 nucleotides, 17-35 nucleotides, 20-35 nucleotides, 25-35 nucleotides, 30-35 nucleotides, 1-35 nucleotides, 2-35 nucleotides, 3-35 nucleotides, 5-35 nucleotides, 7-35 nucleotides, 10-35 nucleotides, 12-35 nucleotides, 15-35 nucleotides, 17-35 nucleotides, 20-35 nucleotides, 25-35 nucleotides, 30-35 nucleotides, 1-40 nucleotides, 2-40 nucleotides, 3-40 nucleotides, 5-40 nucleotides, 7-40 nucleotides, 10-40 nucleotides, 12-40 nucleotides, 15-40 nucleotides, 17-40 residues, 20-40 nucleotides, 25-40 nucleotides, 30-40 nucleotides, 35-40 nucleotides, 1-45 nucleotides, 2-45 nucleotides, 3-45 nucleotides, 5-45 nucleotides, 7-45 nucleotides, 10-45 nucleotides, 12-45 nucleotides, 15-45 nucleotides, 17-45 nucleotides, 20-45 nucleotides, 25-45 nucleotides, 30-45 nucleotides, 35-45 nucleotides, 40-45 nucleotides, 1-50 residues, 2-50 nucleotides, 3-50 nucleotides, 5-50 nucleotides, 7-50 nucleotides, 10-50 nucleotides, 12-50 nucleotides, 15-50 nucleotides, 17-50 nucleotides, 20-50 nucleotides, 25-50 nucleotides, 30-50 nucleotides, 35-50 nucleotides, 40-50 nucleotides, or 45-50 nucleotides, about 1 to about 100 nucleotides, about 50 to about 150 nucleotides, about 100 to about 200 nucleotides, about 150 to about 200 nucleotides, about 150 to about 500 nucleotides, about 500 to about 3000 nucleotides, or about 250 to about 350 nucleotides in length.

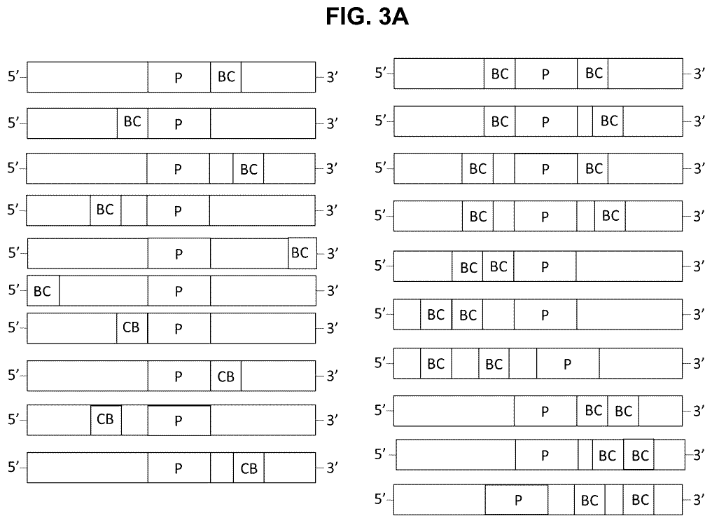

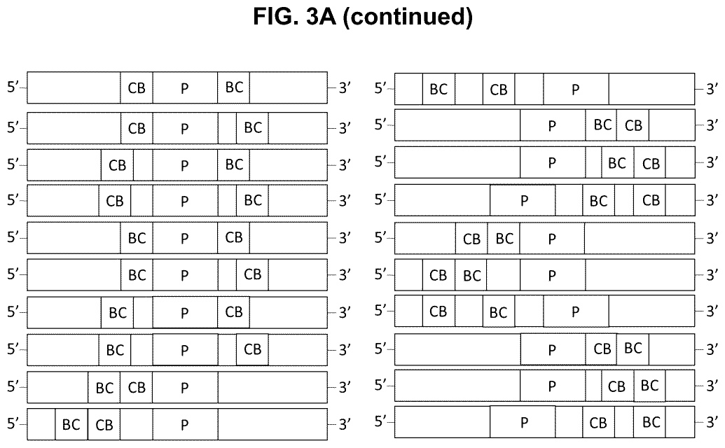

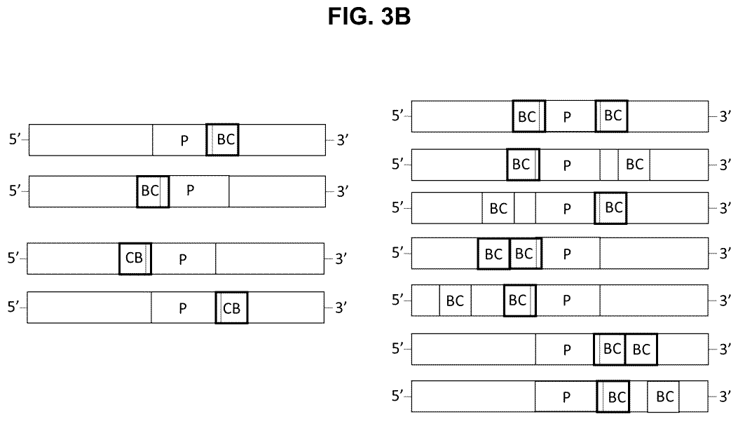

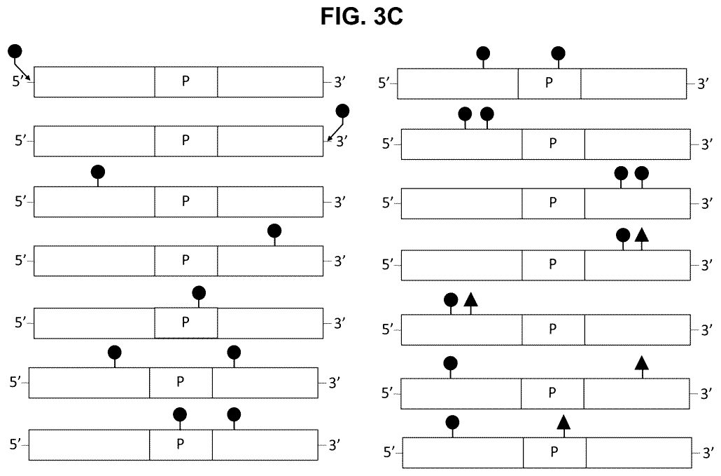

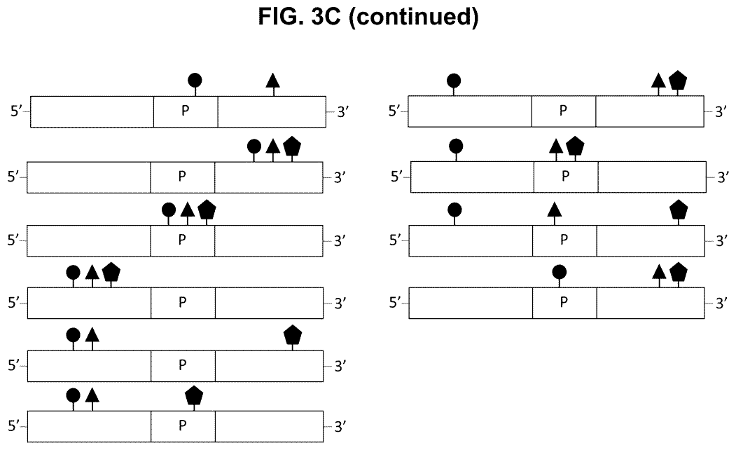

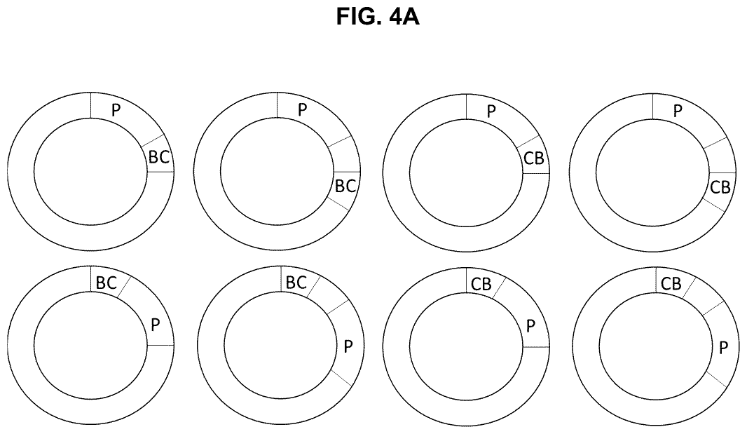

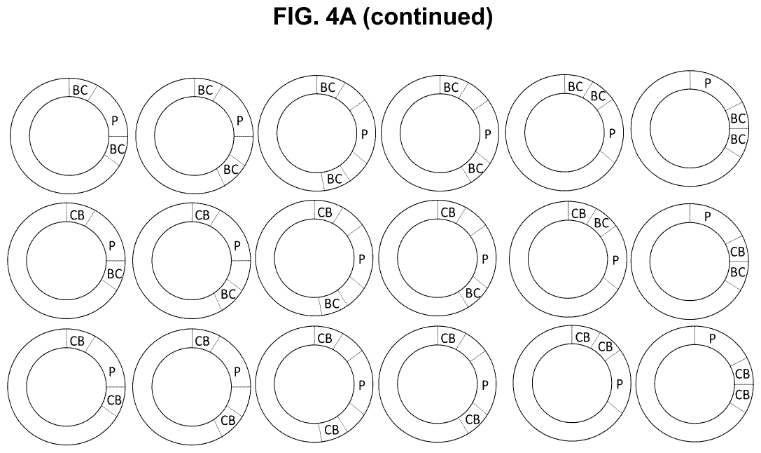

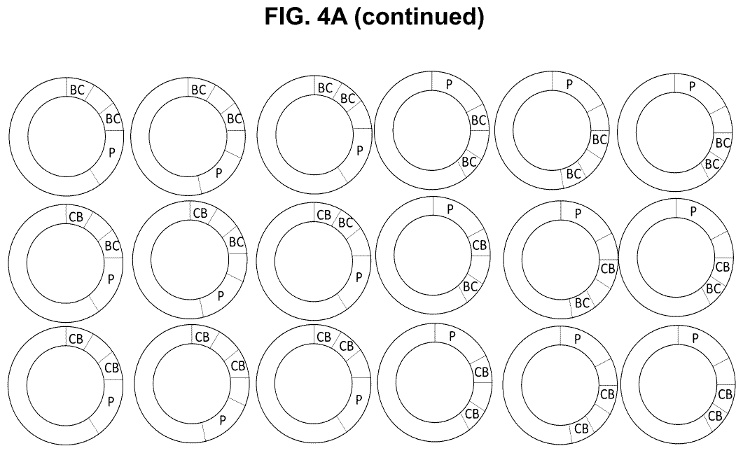

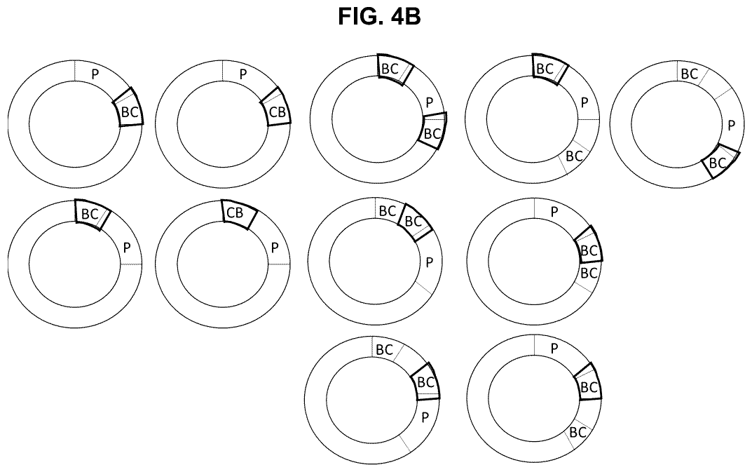

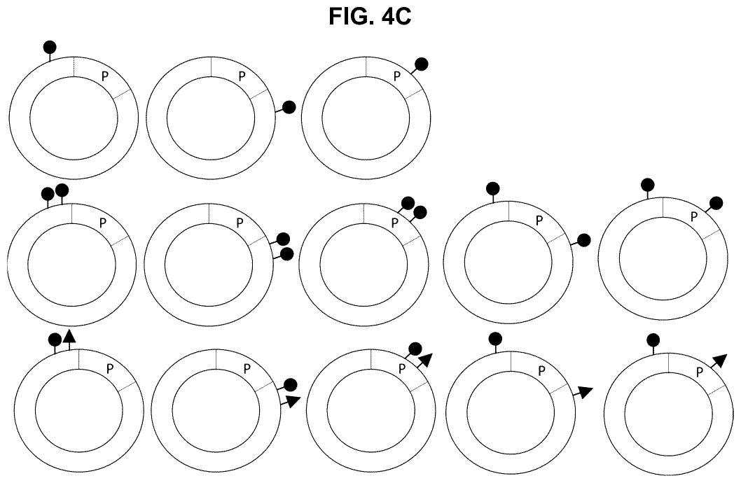

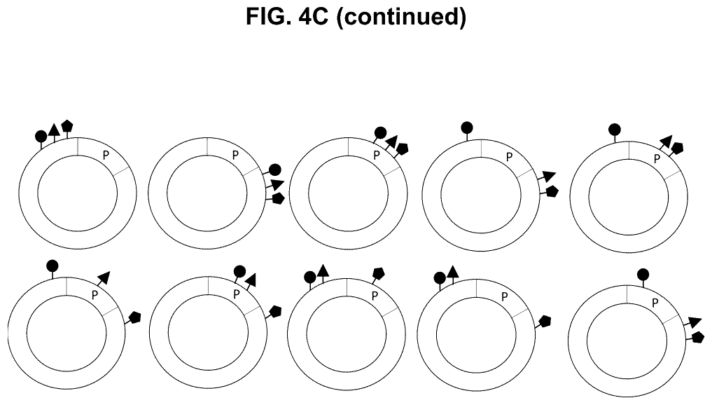

Non-limiting examples of benchmark polynucleotide constructs with at least one identifier, which may be linear or circular, are provided in FIG. 3A, FIG. 3B and FIG. 3C. Non-limiting examples of circular benchmark polynucleotide constructs with at least one identifier are provided in FIG. 4A, FIG. 4B and FIG. 4C. In FIG. 3A, FIG. 3B, FIG. 4A and FIG. 4B the benchmark polynucleotide constructs include a payload region (referred to as “P” in the figure) and at least one identifier region (referred to as “BC” in the figure) and/or an inverted identifier region (referred to as “CB” in the figure). In FIG. 3C and FIG. 4C the benchmark polynucleotide constructs include a payload region (referred to as “P” in the figure) and at least one identifier moiety associated with the benchmark polynucleotide construct.

In some embodiments, the identifier region in the benchmark construct overlaps with the payload region. As used herein, “overlap” means that at least one nucleotide of the identifier region extends into the payload region. In some aspects the identifier region overlaps with the payload region by 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides 41 nucleotides, 42 nucleotides, 43 nucleotides, 44 nucleotides, 45 nucleotides, 46 nucleotides, 47 nucleotides, 48 nucleotides, 49 nucleotides, 50 nucleotides or more than 50 nucleotides. In some aspects the identifier region overlaps with the payload region by 1-5 nucleotides, 2-5 nucleotides, 3-5 nucleotides, 2-7 nucleotides, 3-7 nucleotides, 1-10 nucleotides, 2-10 nucleotides, 3-10 nucleotides, 5-10 nucleotides, 7-10 nucleotides, 1-15 nucleotides, 2-15 nucleotides, 3-15 nucleotides, 5-15 nucleotides, 7-15 nucleotides, 10-15 nucleotides, 12-15 nucleotides, 1-20 nucleotides, 2-20 nucleotides, 3-20 nucleotides, 5-20 nucleotides, 7-20 nucleotides, 10-20 nucleotides, 12-20 nucleotides, 15-20 nucleotides, 17-20 nucleotides, 1-25 nucleotides, 2-25 nucleotides, 3-25 nucleotides, 5-25 nucleotides, 7-25 nucleotides, 10-25 nucleotides, 12-25 nucleotides, 15-25 nucleotides, 17-25 nucleotides, 20-25 nucleotides, 1-30 nucleotides, 2-30 nucleotides, 3-30 nucleotides, 5-30 nucleotides, 7-30 nucleotides, 10-30 nucleotides, 12-30 nucleotides, 15-30 nucleotides, 17-30 nucleotides, 20-30 nucleotides, 25-30 nucleotides, 1-35 nucleotides, 2-35 nucleotides, 3-35 nucleotides, 5-35 nucleotides, 7-35 nucleotides, 10-35 nucleotides, 12-35 nucleotides, 15-35 nucleotides, 17-35 nucleotides, 20-35 nucleotides, 25-35 nucleotides, 30-35 nucleotides, 1-35 nucleotides, 2-35 nucleotides, 3-35 nucleotides, 5-35 nucleotides, 7-35 nucleotides, 10-35 nucleotides, 12-35 nucleotides, 15-35 nucleotides, 17-35 nucleotides, 20-35 nucleotides, 25-35 nucleotides, 30-35 nucleotides, 1-40 nucleotides, 2-40 nucleotides, 3-40 nucleotides, 5-40 nucleotides, 7-40 nucleotides, 10-40 nucleotides, 12-40 nucleotides, 15-40 nucleotides, 17-40 nucleotides, 20-40 nucleotides, 25-40 nucleotides, 30-40 nucleotides, 35-40 nucleotides, 1-45 nucleotides, 2-45 nucleotides, 3-45 nucleotides, 5-45 nucleotides, 7-45 nucleotides, 10-45 nucleotides, 12-45 nucleotides, 15-45 nucleotides, 17-45 nucleotides, 20-45 nucleotides, 25-45 nucleotides, 30-45 nucleotides, 35-45 nucleotides, 40-45 nucleotides, 1-50 nucleotides, 2-50 nucleotides, 3-50 nucleotides, 5-50 nucleotides, 7-50 nucleotides, 10-50 nucleotides, 12-50 nucleotides, 15-50 nucleotides, 17-50 nucleotides, 20-50 nucleotides, 25-50 nucleotides, 30-50 nucleotides, 35-50 nucleotides, 40-50 nucleotides, or 45-50 nucleotides.

In some embodiments, the benchmark polynucleotide construct comprises a payload region and an identifier region. The identifier region may be located 5′ to the payload region, 3′ to the payload region, or the identifier region may overlap with the 5′ end or the 3′end of the payload region.

In some embodiments, the benchmark polynucleotide construct comprises a payload region and two identifier regions. Each identifier region may independently be located 5′ to the payload region, 3′ to the payload region, or the identifier region may overlap with the 5′ end or the 3′ end of the payload region.

As a non-limiting example, the first identifier region is located 5′ to the payload region and the second identifier region is located 3′ to the payload region. As a non-limiting example, the first and second identifier regions are located 5′ to the payload region. As a non-limiting example, the first and second identifier regions are located 3′ to the payload region.

As a non-limiting example, the first identifier region is inverted and is located 5′ to the payload region and the second identifier region is located 3′ to the payload region. As a non-limiting example, the first identifier region is inverted and is located 5′ to the payload region and the second identifier region is inverted and is located 3′ to the payload region. As a non-limiting example, the first identifier region is located 5′ to the payload region and the second identifier region is inverted and is located 3′ to the payload region. As a non-limiting example, the first and second identifier regions are both inverted and are located 5′ to the payload region. As a non-limiting example, the first and second identifier regions are located 5′ to the payload region and the first identifier region is inverted. As a non-limiting example, the first and second identifier regions are located 5′ to the payload region and the second identifier region is inverted. As a non-limiting example, the first and second identifier region are both inverted and located 3′ to the payload region. As a non-limiting example, the first and second identifier regions are located 3′ to the payload region and the first identifier region is inverted. As a non-limiting example, the first and second identifier regions are located 3′ to the payload region and the second identifier region is inverted.

As a non-limiting example, the first identifier region is located 5′ to the payload region and overlaps with the payload region and the second identifier region is located 3′ to the payload region. As a non-limiting example, the first identifier region is located 5′ to the payload region and the second identifier region is located 3′ to the payload region and overlaps with the payload region.

As a non-limiting example, the first and second identifier regions are located 5′ to the payload region and the second identifier region overlaps with the payload region. As a non-limiting example, the first and second identifier regions are located 3′ to the payload region and the first identifier region overlaps with the payload region.

As a non-limiting example, the first identifier region is inverted, is located 5′ to the payload region and overlaps with the payload region, and the second identifier region is located 3′ to the payload region. As a non-limiting example, the first identifier region is inverted and is located 5′ to the payload region and the second identifier region is located 3′ to the payload region and overlaps with the payload region. As a non-limiting example, the first identifier region is inverted, is located 5′ to the payload region, the second identifier region is located 3′ to the payload region, and both of the first and second identifier regions overlap with the payload region.

As a non-limiting example, the first identifier region is inverted, is located 5′ to the payload region and overlaps with the payload region, and the second identifier region is inverted and is located 3′ to the payload region. As a non-limiting example, the first identifier region is inverted and is located 5′ to the payload region and the second identifier region is inverted, is located 3′ to the payload region and overlaps with the payload region. As a non-limiting example, the first identifier region is inverted and is located 5′ to the payload region, and the second identifier region is inverted and is located 3′ to the payload region, and both of the first and second identifier regions overlap with the payload region.

As a non-limiting example, the first identifier region is located 5′ to the payload region and overlaps with the payload region, and the second identifier region is inverted and is located 3′ to the payload region. As a non-limiting example, the first identifier region is located 5′ to the payload region and the second identifier region is inverted, is located 3′ to the payload region and overlaps with the payload region. As a non-limiting example, the first identifier region is located 5′ to the payload region and the second identifier region is inverted and is located 3′ to the payload region, and both of the first and second identifier regions overlap with the payload region.

As a non-limiting example, the first and second identifier regions are both inverted and are located 5′ to the payload region, and the second identifier region overlaps with the payload region. As a non-limiting example, the first and second identifier regions are located 5′ to the payload region and the first identifier region is inverted, and the second identifier region overlaps with the payload region. As a non-limiting example, the first and second identifier regions are located 5′ to the payload region and the second identifier region is inverted and overlaps with the payload region. As a non-limiting example, the first and second identifier region are both inverted and located 3′ to the payload region, and the first identifier region overlap with the payload region. As a non-limiting example, the first and second identifier regions are located 3′ to the payload region and the first identifier region is inverted and overlaps with the payload region. As a non-limiting example, the first and second identifier regions are located 3′ to the payload region and the second identifier region is inverted, and the first payload region overlap with the payload region.

In some embodiments, at least one identifier moiety may be associated with the benchmark polynucleotide construct. The benchmark polynucleotide construct may have 1, 2, 3, 4, 5, 6, 7, 8, 9, or 10 or more identifier moieties associated with the benchmark polynucleotide construct which may be the same moiety or different moieties associated with the benchmark polynucleotide construct. Each identifier moiety may independently be located on the flanking region 5′ to the payload region, on the flanking region 3′ to the payload region, or the location of the identifier moiety may span the 5′ end or the 3′end of the payload region and a flanking region. In some aspects the location of the identifier moiety may include one or more nucleotides of the payload region such as, but not limited to, 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides 41 nucleotides, 42 nucleotides, 43 nucleotides, 44 nucleotides, 45 nucleotides, 46 nucleotides, 47 nucleotides, 48 nucleotides, 49 nucleotides, 50 nucleotides or more than 50 nucleotides. In some aspects the location of the identifier moiety may include one or more nucleotides of the payload region such as, but not limited to, 1-5 nucleotides, 2-5 nucleotides, 3-5 nucleotides, 2-7 nucleotides, 3-7 nucleotides, 1-10 nucleotides, 2-10 nucleotides, 3-10 nucleotides, 5-10 nucleotides, 7-10 nucleotides, 1-15 nucleotides, 2-15 nucleotides, 3-15 nucleotides, 5-15 nucleotides, 7-15 nucleotides, 10-15 nucleotides, 12-15 nucleotides, 1-20 nucleotides, 2-20 nucleotides, 3-20 nucleotides, 5-20 nucleotides, 7-20 nucleotides, 10-20 nucleotides, 12-20 nucleotides, 15-20 nucleotides, 17-20 nucleotides, 1-25 nucleotides, 2-25 nucleotides, 3-25 nucleotides, 5-25 nucleotides, 7-25 nucleotides, 10-25 nucleotides, 12-25 nucleotides, 15-25 nucleotides, 17-25 nucleotides, 20-25 nucleotides, 1-30 nucleotides, 2-30 nucleotides, 3-30 nucleotides, 5-30 nucleotides, 7-30 nucleotides, 10-30 nucleotides, 12-30 nucleotides, 15-30 nucleotides, 17-30 nucleotides, 20-30 nucleotides, 25-30 nucleotides, 1-35 nucleotides, 2-35 nucleotides, 3-35 nucleotides, 5-35 nucleotides, 7-35 nucleotides, 10-35 nucleotides, 12-35 nucleotides, 15-35 nucleotides, 17-35 nucleotides, 20-35 nucleotides, 25-35 nucleotides, 30-35 nucleotides, 1-35 nucleotides, 2-35 nucleotides, 3-35 nucleotides, 5-35 nucleotides, 7-35 nucleotides, 10-35 nucleotides, 12-35 nucleotides, 15-35 nucleotides, 17-35 nucleotides, 20-35 nucleotides, 25-35 nucleotides, 30-35 nucleotides, 1-40 nucleotides, 2-40 nucleotides, 3-40 nucleotides, 5-40 nucleotides, 7-40 nucleotides, 10-40 nucleotides, 12-40 nucleotides, 15-40 nucleotides, 17-40 nucleotides, 20-40 nucleotides, 25-40 nucleotides, 30-40 nucleotides, 35-40 nucleotides, 1-45 nucleotides, 2-45 nucleotides, 3-45 nucleotides, 5-45 nucleotides, 7-45 nucleotides, 10-45 nucleotides, 12-45 nucleotides, 15-45 nucleotides, 17-45 nucleotides, 20-45 nucleotides, 25-45 nucleotides, 30-45 nucleotides, 35-45 nucleotides, 40-45 nucleotides, 1-50 nucleotides, 2-50 nucleotides, 3-50 nucleotides, 5-50 nucleotides, 7-50 nucleotides, 10-50 nucleotides, 12-50 nucleotides, 15-50 nucleotides, 17-50 nucleotides, 20-50 nucleotides, 25-50 nucleotides, 30-50 nucleotides, 35-50 nucleotides, 40-50 nucleotides, or 45-50 nucleotides.

In some embodiments, one identifier moiety may be associated with the benchmark polynucleotide construct. As a non-limiting example, the identifier moiety may be associated with the benchmark polynucleotide construct on the 5′ end of the benchmark polynucleotide construct. As a non-limiting example, the identifier moiety may be associated with the benchmark polynucleotide construct on the 5′ flanking region. As a non-limiting example, the identifier moiety may be associated with the benchmark polynucleotide construct on the 3′ flanking region. As a non-limiting example, the identifier moiety may be associated with the benchmark polynucleotide construct on the 3′ end of the benchmark polynucleotide construct. As a non-limiting example, the identifier moiety may be associated with the benchmark polynucleotide construct on the payload region. As a non-limiting example, the benchmark polynucleotide construct comprises an identifier moiety and the location of the identifier moiety spans the 5′ end of the payload region and the 5′ flanking region. As a non-limiting example, the benchmark polynucleotide construct comprises an identifier moiety and the location of the identifier moiety spans the 3′ end of the payload region and the 3′ flanking region.

In some embodiments, two identifier moieties are associated with the benchmark polynucleotide construct. As a non-limiting example, the first identifier moiety and the second identifier moiety are located on the 5′ flanking region. As a non-limiting example, the first identifier moiety and the second identifier moiety are located on the payload region. As a non-limiting example, the first identifier moiety and the second identifier moiety are located on the 3′ flanking region. As a non-limiting example, the first identifier moiety and the second identifier moiety are located on the 5′ end of the benchmark polynucleotide construct. As a non-limiting example, the first identifier moiety and the second identifier moiety are located on the 3′ end of the benchmark polynucleotide construct.

As a non-limiting example, the first identifier moiety is located on the 5′ end of the benchmark polynucleotide construct and the second identifier moiety is located on the 5′ flanking region. As a non-limiting example, the first identifier moiety is located on the 5′ end of the benchmark polynucleotide construct and the second identifier moiety is located on the payload region. As a non-limiting example, the first identifier moiety is located on the 5′ end of the benchmark polynucleotide construct and the second identifier moiety is located on the 3′ flanking region. As a non-limiting example, the first identifier moiety is located on the 5′ end of the benchmark polynucleotide construct and the location of the second identifier moiety spans the 5′ flanking region and the payload region. As a non-limiting example, the first identifier moiety is located on the 5′ end of the benchmark polynucleotide construct and the location of the second identifier moiety spans the 3′ flanking region and the payload region. As a non-limiting example, the first identifier moiety is located on the 5′ end of the benchmark polynucleotide construct and the second identifier moiety is located on the 3′ end of the benchmark polynucleotide construct.

As a non-limiting example, the first identifier moiety is located on the 5′ flanking region and the second identifier moiety is located on the payload region. As a non-limiting example, the first identifier moiety is located on the 5′ flanking region and the second identifier moiety is located on the 3′ flanking region. As a non-limiting example, the first identifier moiety is located on the 5′ flanking region and the location of the second identifier moiety spans the 5′ flanking region and the payload region. As a non-limiting example, the first identifier moiety is located on the 5′ flanking region and the location of the second identifier moiety spans the 3′ flanking region and the payload region. As a non-limiting example, the first identifier moiety is located on the 5′ flanking region and the second identifier moiety is located on the 5′ end of the benchmark polynucleotide construct. As a non-limiting example, the first identifier moiety is located on the 5′ flanking region and the second identifier moiety is located on the 3′ end of the benchmark polynucleotide construct.

As a non-limiting example, the location of the first identifier moiety spans the 5′ flanking region and the payload region and the second identifier moiety is located on the 5′ end of the benchmark polynucleotide construct. As a non-limiting example, the location of the first identifier moiety spans the 5′ flanking region and the payload region and the second identifier moiety is located on the 5′ flanking region. As a non-limiting example, the location of the first identifier moiety spans the 5′ flanking region and the payload region and the second identifier moiety is located on the payload region. As a non-limiting example, the location of the first identifier moiety spans the 5′ flanking region and the payload region and the location of the second identifier moiety spans the 3′ flanking region and the payload region. As a non-limiting example, the location of the first identifier moiety spans the 5′ flanking region and the payload region and the second identifier moiety is located on the 3′ flanking region. As a non-limiting example, the location of the first identifier moiety spans the 5′ flanking region and the payload region and the second identifier moiety is located on the 3′ end of the benchmark polynucleotide construct.

As a non-limiting example, the first identifier moiety is located on the payload region and the second identifier moiety is located on the 5′ end of the benchmark polynucleotide construct. As a non-limiting example, the first identifier moiety is located on the payload region and the second identifier moiety is located on the 5′ flanking region. As a non-limiting example, the first identifier moiety is located on the payload region and the location of the second identifier moiety spans the 5′ flanking region and the payload region. As a non-limiting example, the first identifier moiety is located on the payload region and the location of the second identifier moiety spans the 3′ flanking region and the payload region. As a non-limiting example, the first identifier moiety is located on the payload region and the second identifier moiety is located on the 3′ flanking region. As a non-limiting example, the first identifier moiety is located on the payload region and the second identifier moiety is located on the 3′ end of the benchmark polynucleotide construct.

As a non-limiting example, the location of the first identifier moiety spans the 3′ flanking region and the payload region and the second identifier moiety is located on the 5′ end of the benchmark polynucleotide construct. As a non-limiting example, the location of the first identifier moiety spans the 3′ flanking region and the payload region and the second identifier moiety is located on the 5′ flanking region. As a non-limiting example, the location of the first identifier moiety spans the 3′ flanking region and the payload region and the location of the second identifier moiety spans the 5′ flanking region and the payload region. As a non-limiting example, the location of the first identifier moiety spans the 3′ flanking region and the payload region and the second identifier moiety is located on the payload region. As a non-limiting example, the location of the first identifier moiety spans the 3′ flanking region and the payload region and the second identifier moiety is located on the 3′ flanking region. As a non-limiting example, the location of the first identifier moiety spans the 3′ flanking region and the payload region and the second identifier moiety is located on the 3′end of the benchmark polynucleotide construct.

As a non-limiting example, the location of the first identifier moiety spans the 3′ flanking region and the payload region and the second identifier moiety is located on the 5′ flanking region. As a non-limiting example, the location of the first identifier moiety spans the 5′ flanking region and the payload region and the second identifier moiety is located on the payload region. As a non-limiting example, the location of the first identifier moiety spans the 5′ flanking region and the payload region and the location of the second identifier moiety spans the 3′ flanking region and the payload region. As a non-limiting example, the location of the first identifier moiety spans the 5′ flanking region and the payload region and the second identifier moiety is located on the 3′ flanking region. As a non-limiting example, the location of the first identifier moiety spans the 5′ flanking region and the payload region and the second identifier moiety is located on the 3′ end of the benchmark polynucleotide construct.

As a non-limiting example, the first identifier moiety is located on the 3′ flanking region and the second identifier moiety is located on the 5′ end of the benchmark polynucleotide construct. As a non-limiting example, the first identifier moiety is located on the 3′ flanking region and the second identifier moiety is located on the 5′ flanking region. As a non-limiting example, the first identifier moiety is located on the 3′ flanking region and the location of the second identifier moiety spans the 5′ flanking region and the payload region. As a non-limiting example, the first identifier moiety is located on the 3′ flanking region and the second identifier moiety is located on the payload region. As a non-limiting example, the first identifier moiety is located on the 3′ flanking region and the location of the second identifier moiety spans the 3′ flanking region and the payload region. As a non-limiting example, the first identifier moiety is located on the 3′ flanking region and the second identifier moiety is located on the 3′ end of the benchmark polynucleotide construct.

As a non-limiting example, the first identifier moiety is located on the 3′ end of the benchmark polynucleotide construct and the second identifier moiety is located on the 5′ end of the benchmark polynucleotide construct. As a non-limiting example, the first identifier moiety is located on the 3′ end of the benchmark polynucleotide construct and the second identifier moiety is located on the 5′ flanking region. As a non-limiting example, the first identifier moiety is located on the 5′ end of the benchmark polynucleotide construct and the location of the second identifier moiety spans the 5′ flanking region and the payload region. As a non-limiting example, the first identifier moiety is located on the 3′ end of the benchmark polynucleotide construct and the second identifier moiety is located on the payload region. As a non-limiting example, the first identifier moiety is located on the 5′ end of the benchmark polynucleotide construct and the location of the second identifier moiety spans the 3′ flanking region and the payload region. As a non-limiting example, the first identifier moiety is located on the 3′ end of the benchmark polynucleotide construct and the second identifier moiety is located on the 3′ flanking region.

In some embodiments, three identifier moieties are associated with the benchmark polynucleotide construct.

In some embodiments, four identifier moieties are associated with the benchmark polynucleotide construct.

In some embodiments, five identifier moieties are associated with the benchmark polynucleotide construct.

In some embodiments, six identifier moieties are associated with the benchmark polynucleotide construct.

In some embodiments, seven identifier moieties are associated with the benchmark polynucleotide construct.

In some embodiments, eight identifier moieties are associated with the benchmark polynucleotide construct.

In some embodiments, nine identifier moieties are associated with the benchmark polynucleotide construct.

In some embodiments, ten identifier moieties are associated with the benchmark polynucleotide construct.

### II. Cargo and Payloads

The originator constructs and benchmark constructs of the present disclosure may comprise, encode or be conjugated to a cargo or payload. As used herein, the term “cargo” or “payload” can refer to one or more molecules or structures encompassed in a pharmaceutical delivery vehicle for delivery to or into a cell or tissue. Non-limiting examples of cargo can include a nucleic acid, a polypeptide, peptide, protein, a liposome, a label, a tag, a small chemical molecule, a large biological molecule, and any combinations or fragments thereof. In the originator constructs and benchmark constructs, the region of the construct which comprises or encodes the cargo or payload is referred to as the “cargo region” or the “payload region.”

In some embodiments, the cargo or payload is or encodes a biologically active molecule such as, but not limited to a therapeutic protein. As used herein, the term “biologically active” refers to a characteristic of any agent that has activity in a biological system, and particularly in an organism. For instance, an agent that, when administered to an organism, has a biological effect on that organism, is considered to be biologically active. In some embodiments, the cargo or payload is or encodes one or more prophylactically- or therapeutically-active proteins, polypeptides, or other factors. As a non-limiting example, the cargo or payload may be or encode an agent that enhances tumor killing activity such as, but not limited to, TRAIL or tumor necrosis factor (TNF), in a cancer. As another non-limiting example, the cargo or payload may be or encode an agent suitable for the treatment of conditions such as muscular dystrophy (e.g., cargo or payload is or encodes Dystrophin), cardiovascular disease (e.g., cargo or payload is or encodes SERCA2a, GATA4, Tbx5, Mef2C, Hand2, Myocd, etc.), neurodegenerative disease (e.g., cargo or payload is or encodes NGF, BDNF, GDNF, NT-3, etc.), chronic pain (e.g., cargo or payload is or encodes GlyRal), an enkephalin, or a glutamate decarboxylase (e.g., cargo or payload is or encodes GAD65, GAD67, or another isoform), lung disease (e.g., cargo or payload is or encodes CFTR), hemophilia (e.g., cargo or payload is or encodes Factor VIII or Factor IX), neoplasia (e.g., cargo or payload is or encodes PTEN, ATM, ATR, EGFR, ERBB2, ERBB3, ERBB4, Notch1, Notch2, Notch3, Notch4, AKT, AKT2, AKT3, HIF, HI Fla, HIF3a, Met, HRG, Bcl2, PPARalpha, PPAR gamma, WT1 (Wilms Tumor), FGF Receptor Family members (5 members: 1, 2, 3, 4, 5), CDKN2a, APC, RB (retinoblastoma), MEN1, VHL, BRCA1, BRCA2, AR (androgen receptor), TSG101, IGF, IGF receptor, Igf1 (4 variants), Igf2 (3 variants), Igf1 receptor, Igf2 receptor, Bax, Bcl2, caspases family (9 members: 1, 2, 3, 4, 6, 7, 8, 9, 12), Kras, Ape), age-related macular degeneration (e.g., cargo or payload is or encodes Aber, Ccl2, Cc2, cp (ceruloplasmin), Timp3, cathepsin D, Vldlr), schizophrenia (e.g. Neuregulin (Nrgl), Erb4 (receptor for Neuregulin), Complexin-1 (Cplx1), Tph1 (tryptophan hydroxylase), Tph2 (tryptophan hydroxylase 2), neurexin 1, GSK3, GSK3a, GSK3b, 5-HIT (Slc6a4), COMT, DRD (Drdla), SLC6A3, DAOA, DTNBPI, Dao (Daol)), trinucleotide repeat disorders (e.g., HTT (Huntington's Dx), SBMA/SMAXI/AR (Kennedy's Dx), FXN/X25 (Friedrich's Ataxia), ATX3 (Machado-Joseph's Dx), ATXNI and ATXN2 (spinocerebellar ataxias), DMPK (myotonic dystrophy), Atrophin-1 and Atnl(DRPLA Dx), CBP (Creb-BP-global instability), VLDLR (Alzheimer's), Atxn7, Atxn10), fragile X syndrome (e.g., cargo or payload is or encodes FMR2, FXRI, FXR2, mGLUR5), secretase related disorders (e.g., cargo or payload is or encodes APH-1 (alpha and beta), Presenilin (Psenl), nicastrin (Ncstn), PEN-2), ALS (e.g., cargo or payload is or encodes SOD1, ALS2, STEX, FUS, TARD BP, VEGF (VEGF-a, VEGF-b, VEGF-c)), autism (e.g., cargo or payload is or encodes Mecp2, BZRAP1, MDGA2, Sema5A, Neurexin 1), Alzheimer's disease (e.g., cargo or payload is or encodes E1, CHIP, UCH, UBB, Tau, LRP, PICALM, Clusterin, PSi, SORL1, CR1, Vldlr, Ubal, Uba3, CHIP28 (Aqpl, Aquaporin 1), Uchll, Uchl3, APP), inflammation (e.g., cargo or payload is or encodes IL-10, IL-1 (IL-Ia, IL-Ib), IL-13, IL-17 (IL-17a (CTLA8), IL-17b, IL-17c, IL-17d, IL-171), IL-23, Cx3crl, ptpn22, TNFa, NOD2/CARD15 for IBD, IL-6, IL-12 (IL-12a, IL-12b), CTLA4, Cx3cll), Parkinson's Disease (e.g., x-Synuclein, DJ-1, LRRK2, Parkin, PINK1), blood and coagulation disorders, such as, e.g., anemia, bare lymphocyte syndrome, bleeding disorders, hemophagocytic lymphohistiocytosis disorders, hemophilia A, hemophilia B, hemorrhagic disorders, leukocyte deficiencies and disorders, sickle cell anemia, and thalassemia (e.g., cargo or payload is or encodes CRAN1, CDA1, RPS19, DBA, PKLR, PK1, NT5C3, UMPH1, PSNI, RHAG, RH50A, NRAMP2, SPTB, ALAS2, ANH1, ASB, ABCB7, ABC7, ASAT, TAPBP, TPSN, TAP2, ABCB3, PSF2, RING11, MHC2TA, C2TA, RFX5, RFXAP, RFX5, TBXA2R, P2RX1, P2X1, HF1, CFH, HUS, MCFD2, FANCA, FAC A, FA1, FA, FA A, FAAP95, FAAP90, FLJ34064, FANCB, FANCC, FACC, BRCA2, FANCDI, FANCD2, FANCD, FACD, FAD, FANCE, FACE, FANCF, XRCC9, FANCG, BR1PI, BACH1, FANCJ, PHF9, FANCL, FANCM, KIAA1596, PRF1, HPLH2, UNC13D, MUNC13-4, HPLH3, HLH3, FHL3, F8, FSC, PI, ATT, F5, ITGB2, CD18, LCAMB, LAD, EIF2B1, EIF2BA, EIF2B2, EIF2B3, EIF2B5, LVWM, CACH, CLE, EIF2B4, HBB, HBA2, HBB, HBD, LCRB, HBA1), B-cell non-Hodgkin lymphoma or leukemia (e.g., cargo or payload is or encodes BCL7A, BCL7, ALI, TCL5, SCL, TAL2, FLT3, NBS1, NBS, ZNFN1AI, 1KI, LYF1, HOXD4, HOX4B, BCR, CML, PHL, ALL, ARNT, KRAS2, RASK2, GMPS, AFIO, ARHGEF12, LARG, KIAA0382, CALM, CLTH, CEBPA, CEBP, CHIC2, BTL, FLT3, KIT, PBT, LPP, NPMI, NUP214, D9S46E, CAN, CAIN, RUNXI, CBFA2, AML1, WHSClLI, NSD3, FLT3, AF1Q, NPMI, NUMA1, ZNF145, PLZF, PML, MYL, STAT5B, AF1Q, CALM, CLTH, ARL11, ARLTS1, P2RX7, P2X7, BCR, CML, PHL, ALL, GRAF, NF1, VRNF, WSS, NFNS, PTPNII, PTP2C, SHP2, NS1, BCL2, CCND1, PRAD1, BCL1, TCRA, GATA1, GF1, ERYF1, NFE1, ABLI, NQO1, DIA4, NMOR1, NUP214, D9S46E, CAN, CAIN), inflammation and immune related diseases and disorders (e.g., cargo or payload is or encodes KIR3DL1, NKAT3, NKB1, AMB11, K1R3DS1, IFNG, CXCL12, TNFRSF6, APT1, FAS, CD95, ALPS1A, IL2RG, SCIDX1, SCIDX, IMD4, CCL5, SCYA5, D17S136E, TCP228, IL10, CSIF, CMKBR2, CCR2, CMKBR5, CCCKR5 (CCR5), CD3E, CD3G, AICDA, AID, HIGM2, TNFRSF5, CD40, UNG, DGU, HIGM4, TNFSFS, CD40LG, HIGM1, IGM, FOXP3, IPEX, AIID, XPID, PIDX, TNFRSF14B, TACI), inflammation (e.g., cargo or payload is or encodes IL-10, IL-1 (IL-IA, IL-IB), IL-13, IL-17 (IL-17a (CTLA8), IL-17b, IL-17c, IL-17d, IL-171), 11-23, Cx3crl, ptpn22, TNFa, NOD2/CARD15 for IBD, IL-6, IL-12 (IL-12a, IL-12b), CTLA4, Cx3cII), JAK3, JAKL, DCLREIC, ARTEMIS, SCIDA, RAG1, RAG2, ADA, PTPRC, CD45, LCA, IL7R, CD3D, T3D, IL2RG, SCIDXI, SCIDX, IMD4), metabolic, liver, kidney and protein diseases and disorders (e.g., cargo or payload is or encodes TTR, PALB, APOA1, APP, AAA, CVAP, ADI, GSN, FGA, LYZ, TTR, PALB, KRT18, KRT8, CIRHIA, NAIC, TEX292, KIAA1988, CFTR, ABCC7, CF, MRP7, SLC2A2, GLUT2, G6PC, G6PT, G6PT1, GAA, LAMP2, LAMPB, AGL, GDE, GBE1, GYS2, PYGL, PFKM, TCF1, HNF1A, MODY3, SCOD1, SCOl, CTNNB1, PDGFRL, PDGRL, PRLTS, AX1NI, AXIN, CTNNB1, TP53, P53, LFS1, IGF2R, MPRI, MET, CASP8, MCH5, UMOD, HNFJ, FJHN, MCKD2, ADMCKD2, PAH, PKU1, QDPR, DHPR, PTS, FCYT, PKHD1, ARPKD, PKD1, PKD2, PKD4, PKDTS, PRKCSH, G19P1, PCLD, SEC63), muscular/skeletal diseases and disorders (e.g., cargo or payload is or encodes DMD, BMD, MYF6, LMNA, LMN1, EMD2, FPLD, CMDIA, HGPS, LGMDIB, LMNA, LMNI, EMD2, FPLD, CMDIA, FSHMD1A, FSHD1A, FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDCIC, LGMD21, TTN, CMD1G, TMD, LGMD2J, POMT1, CAV3, LGMD1C, SEPN1, SELN, RSMD1, PLEC1, PLTN, EBS1, LRP5, BMND1, LRP7, LR3, OPPG, VBCH2, CLCN7, CLC7, OPTA2, OSTMI, GL, TCIRG1, TIRC7, OC116, OPTB1, VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, SMARD1), neurological and neuronal diseases and disorders (e.g., cargo or payload is or encodes SOD1, ALS2, STEX, FUS, TARDBP, VEGF (VEGF-a, VEGF-b, VEGF-c), APP, AAA, CVAP, ADI, APOE, AD2, PSEN2, AD4, STM2, APBB2, FE65LI, NOS3, PLAU, URK, ACE, DCPI, ACEI, MPO, PACIPI, PAXIPIL, PTIP, A2M, BLMH, BMH, PSEN1, AD3, Mecp2, BZRAP1, MDGA2, Sema5A, Neurexin 1, GLOl, MECP2, RTT, PPMX, MRX16, MRX79, NLGN3, NLGN4, KIAA1260, AUTSX2, FMR2, FXR1, FXR2, mGLUR5, HD, IT15, PRNP, PRIP, JPH3, JP3, HDL2, TBP, SCA17, NR4A2, NURR1, NOT, TINUR, SNCAIP, TBP, SCA17, SNCA, NACP, PARK1, PARK4, DJI, PARK7, LRRK2, PARK8, PINK1, PARK6, UCHL1, PARK5, SNCA, NACP, PARK1, PARK4, PRKN, PARK2, PDJ, DBH, NDUFV2, MECP2, RTT, PPMX, MRX16, MRX79, CDKL5, STK9, MECP2, RTT, PPMX, MRX16,MRX79, x-Synuclein, DJ-1, Neuregulin-1 (Nrgl), Erb4 (receptor for Neuregulin), Complexin-1 (Cplx1), Tph1 Tryptophan hydroxylase, Tph2, Tryptophan hydroxylase 2, Neurexin 1, GSK3, GSK3a, GSK3b, 5-HTT (Slc6a4), CONT, DRD (Drdla), SLC6A, DAOA, DTNBP1, Dao (Daol), APH-1(alpha and beta), Presenilin (Psenl), Nicastrin, (Ncstn), PEN-2, Nosl, Parpl, Nat1, Nat2, HTT, SBMA/SMAX1/AR, FXN/X25, ATX3, TXN, ATXN2, DMPK, Atrophin-1, Atnl, CBP, VLDLR, Atxn7, and Atxn10), and ocular diseases and disorders (e.g., Aber, Ccl2, Cc2, cp (ceruloplasmin), Timp3, cathepsin-D, Vldlr, Ccr2, CRYAA, CRYA1, CRYBB2, CRYB2, PITX3, BFSP2, CP49, CP47, CRYAA, CRYAI, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYAI, GJA8, CX50, CAEl, GJA3, CX46, CZP3, CAE3, CCM1, CAM, KRIT1, APOA1, TGFBI, CSD2, CDGG1, CSD, BIGH3, CDG2, TACSTD2, TROP2, MlSI, VSX1, RINX, PPCD, PPD, KTCN, COL8A2, FECD, PPCD2, PIP5K3, CFD, KERA, CNA2, MYOC, TIGR, GLCIA, JO AG, GPOA, OPTN, GLC1E, FIP2, HYPL, NRP, CYPIBI, GLC3A, OPAl, NTG, NPG, CYPIBI, GLC3A, CRB1, RP12, CRX, CORD2, CRD, RPGRIPI, LCA6, CORD9, RPE65, RP20, AIPL1, LCA4, GUCY2D, GUC2D, LCA1, CORD6, RDH12, LCA3, ELOVL4, ADMD, STGD2, STGD3, RDS, RP7, PRPH2, PRPH, AVMD, AOFMD, and VMD2).

In some embodiments, the cargo or payload is or encodes a factor that can affect the differentiation of a cell. As a non-limiting example, the expression of one or more of Oct4, Klf4, Sox2, c-Myc, L-Myc, dominant-negative p53, Nanog, Glisl, Lin28, TFIID, mir-302/367, or other miRNAs can cause the cell to become an induced pluripotent stem (iPS) cell.

In some embodiments, the cargo or payload is or encodes a factor for transdifferentiating cells. Non-limiting examples of factors include: one or more of GATA4, Tbx5, Mef2C, Myocd, Hand2, SRF, Mespl, SMARCD3 for cardiomyocytes; Ascii, Nurrl, LmxlA, Bm2, Mytll, NeuroDl, FoxA2 for neural cells; and Hnf4a, Foxal, Foxa2 or Foxa3 for hepatic cells.

a. Polypeptides, Proteins and Peptides

The originator constructs and benchmark constructs of the present disclosure may comprise, encode or be conjugated to a cargo or payload which is a polypeptide, protein or peptide. As used herein, the term “polypeptide” generally refers to polymers of amino acids linked by peptide bonds and embraces “protein” and “peptides.” Polypeptides for the present disclosure include all polypeptides, proteins and/or peptides known in the art. Non-limiting categories of polypeptides include antigens, antibodies, antibody fragments, cytokines, peptides, hormones, enzymes, oxidants, antioxidants, synthetic polypeptides, and chimeric polypeptides.

As used herein, the term “peptide” generally refers to shorter polypeptides of about 50 amino acids or less. Peptides with only two amino acids may be referred to as “dipeptides.” Peptides with only three amino acids may be referred to as “tripeptides.” Polypeptides generally refer to polypeptides with from about 4 to about 50 amino acids. Peptides may be obtained via any method known to those skilled in the art. In some embodiments, peptides may be expressed in culture. In some embodiments, peptides may be obtained via chemical synthesis (e.g. solid phase peptide synthesis).

In some embodiments, the originator constructs and benchmark constructs of the present disclosure may comprise, encode or be conjugated to a cargo or payload which is a simple protein which upon hydrolysis yields the amino acids and occasionally small carbohydrate compounds. Non-limiting examples of simple proteins include albumins, albuminoids, globulins, glutelins, histones and protamines.

In some embodiments, the originator constructs and benchmark constructs of the present disclosure may comprise, encode or be conjugated to a cargo or payload which is a conjugated protein which may be a simple protein associated with a non-protein. Non-limiting examples of conjugated proteins include, glycoproteins, hemoglobins, lecithoproteins, nucleoproteins, and phosphoproteins.

In some embodiments, the originator constructs and benchmark constructs of the present disclosure may comprise, encode or be conjugated to a cargo or payload which is a derived protein which is a protein that is derived from a simple or conjugated protein by chemical or physical means. Non-limiting examples of derived proteins include denatured proteins and peptides.

In some embodiments, the polypeptide, protein or peptide may be unmodified.

In some embodiments, the polypeptide, protein or peptide may be modified. Types of modifications include, but are not limited to, phosphorylation, glycosylation, acetylation, ubiquitylation/sumoylation, methylation, palmitoylation, quinone, amidation, myristoylation, pyrrolidone carboxylic acid, hydroxylation, phosphopantetheine, prenylation, GPI anchoring, oxidation, ADP-ribosylation, sulfation, S-nitrosylation, citrullination, nitration, gamma-carboxyglutamic acid, formylation, hypusine, topaquinone (TPQ), bromination, lysine topaquinone (LTQ), tryptophan tryptophylquinone (TTQ), iodination, and cysteine tryptophylquinone (CTQ). In some aspects, the polypeptide, protein or peptide may be modified by a post-transcriptional modification which can affect its structure, subcellular localization, and/or function.

In some embodiments, the polypeptide, protein or peptide may be modified using phosphorylation. Phosphorylation, or the addition of a phosphate group to serine, threonine, or tyrosine residues, is one of most common forms of protein modification. Protein phosphorylation plays an important role in fine tuning the signal in the intracellular signaling cascades.

In some embodiments, the polypeptide, protein or peptide may be modified using ubiquitination which is the covalent attachment of ubiquitin to target proteins. Ubiquitination-mediated protein turnover has been shown to play a role in driving the cell cycle as well as in protein-degradation-independent intracellular signaling pathways.

In some embodiments, the polypeptide, protein or peptide may be modified using acetylation and methylation which can play a role in regulating gene expression. As a non-limiting example, the acetylation and methylation could mediate the formation of chromatin domains (e.g., euchromatin and heterochromatin) which could have an impact on mediating gene silencing.

In some embodiments, the polypeptide, protein or peptide may be modified using glycosylation. Glycosylation is the attachment of one of a large number of glycan groups and is a modification that occurs in about half of all proteins and plays a role in biological processes including, but not limited to, embryonic development, cell division, and regulation of protein structure. The two main types of protein glycosylation are N-glycosylation and O-glycosylation. For N-glycosylation the glycan is attached to an asparagine and for O-glycosylation the glycan is attached to a serine or threonine.

In some embodiments, the polypeptide, protein or peptide may be modified using sumoylation. Sumoylation is the addition of SUMOs (small ubiquitin-like modifiers) to proteins and is a post-translational modification similar to ubiquitination.

b. Antibodies

As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional”). Antibodies are primarily amino acid based molecules which are monomeric or multimeric. The antibodies may comprise one or more modifications (including, but not limited to the addition of sugar moieties, fluorescent moieties, chemical tags, etc.). For the purposes herein, an “antibody” may comprise a heavy and light variable domain as well as an Fc region.

The cargo or payload may comprise or may encode polypeptides that form one or more functional antibodies.

In some embodiments, the cargo or payload may comprise or may encode polypeptides that form or function as any antibody including, but not limited to, antibodies that are known in the art and/or antibodies that are commercially available which may be therapeutic, diagnostic, or for research purposes. Additionally, the cargo or payload may comprise or may encode fragments of such antibodies or antibodies such as, but not limited to, variable domains or complementarity determining regions (CDRs).

As used herein, the term “native antibody” refers to an usually heterotetrameric glycoprotein of about 150,000 Daltons, composed of two identical light (L) chains and two identical heavy (H) chains. Genes encoding antibody heavy and light chains are known and segments making up each have been well characterized and described (Matsuda, F. et al., 1998. The Journal of Experimental Medicine. 188(11); 2151-62 and Li, A. et al., 2004. Blood. 103(12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments can also be used based on comparisons with other antibodies (Strohl, W.R. Therapeutic Antibody Engineering. Woodhead Publishing, Philadelphia PA. 2012. Ch. 3, p47-54, the contents of which are herein incorporated by reference in their entirety). Determining residues making up CDRs may include the use of numbering schemes including, but not limited to, those taught by Kabat [Wu, T.T. et al., 1970, JEM, 132(2):211-50 and Johnson, G. et al., 2000, Nucleic Acids Res. 28(1): 214-8, the contents of each of which are herein incorporated by reference in their entirety], Chothia [Chothia and Lesk, J. Mol. Biol. 196, 901 (1987), Chothia et al., Nature 342, 877 (1989) and A1-Lazikani, B. et al., 1997, J. Mol. Biol. 273(4):927-48, the contents of each of which are herein incorporated by reference in their entirety], Lefranc (Lefranc, M. P. et al., 2005, Immunome Res. 1:3) and Honegger (Honegger, A. and Pluckthun, A. 2001. J. Mol. Biol. 309(3):657-70, the contents of which are herein incorporated by reference in their entirety).

V_{H} and V_{L} domains each have three CDRs. V_{L} CDRs are referred to herein as CDR-L1, CDR-L2 and CDR-L3, in order of occurrence when moving from N- to C-terminus along the variable domain polypeptide. V_{H} CDRs are referred to herein as CDR-H1, CDR-H2, and CDR-H3, in order of occurrence when moving from N- to C-terminus along the variable domain polypeptide. Each of the CDRs have favored canonical structures with the exception of the CDR-H3, which comprises amino acid sequences that may be highly variable in sequence and length between antibodies resulting in a variety of three-dimensional structures in antigen-binding domains. In some cases, CDR-H3s may be analyzed among a panel of related antibodies to assess antibody diversity.

Various methods of determining CDR sequences are known in the art and may be applied to known antibody sequences. The system described by Kabat, also referred to as “numbered according to Kabat,” “Kabat numbering,” “Kabat definitions,” and “Kabat labeling,” provides an unambiguous residue numbering system applicable to any variable domain of an antibody, and provides precise residue boundaries defining the three CDRs of each chain. (Kabat et al., Sequences of Proteins of Immunological Interest, National Institutes of Health, Bethesda, Md. (1987) and (1991), the contents of which are incorporated by reference in their entirety). Kabat CDRs and comprise about residues 24-34 (CDR1), 50-56 (CDR2) and 89-97 (CDR3) in the light chain variable domain, and 31-35 (CDR1), 50-65 (CDR2) and 95-102 (CDR3) in the heavy chain variable domain. Chothia and coworkers found that certain sub-portions within Kabat CDRs adopt nearly identical peptide backbone conformations, despite having great diversity at the level of amino acid sequence. (Chothia et al. (1987) J. Mol. Biol. 196: 901-917; and Chothia et al. (1989) Nature 342: 877-883, the contents of each of which is herein incorporated by reference in its entirety). These CDRs can be referred to as “Chothia CDRs,” “Chothia numbering,” or “numbered according to Chothia,” and comprise about residues 24-34 (CDR1), 50-56 (CDR2) and 89-97 (CDR3) in the light chain variable domain, and 26-32 (CDR1), 52-56 (CDR2) and 95-102 (CDR3) in the heavy chain variable domain. Mol. Biol. 196:901-917 (1987). The system described by MacCallum, also referred to as “numbered according to MacCallum,” or “MacCallum numbering” comprises about residues 30-36 (CDR1), 46-55 (CDR2) and 89-96 (CDR3) in the light chain variable domain, and 30-35 (CDR1), 47-58 (CDR2) and 93-101 (CDR3) in the heavy chain variable domain. (MacCallum et al. ((1996) J. Mol. Biol. 262(5):732-745), the contents of which is herein incorporated by reference in its entirety). The system described by AbM, also referred to as “numbering according to AbM,” or “AbM numbering” comprises about residues 24-34 (CDR1), 50-56 (CDR2) and 89-97 (CDR3) in the light chain variable domain, and 26-35 (CDR1), 50-58 (CDR2) and 95-102 (CDR3) in the heavy chain variable domain. The IMGT (INTERNATIONAL IMMUNOGENETICS INFORMATION SYSTEM) numbering of variable regions can also be used, which is the numbering of the residues in an immunoglobulin variable heavy or light chain according to the methods of the IIMGT (Lefranc, M.-P., “The IMGT unique numbering for immunoglobulins, T cell Receptors and Ig-like domains”, The Immunologist, 7, 132-136 (1999), and is herein incorporated by reference in its entirety by reference). As used herein, “IMGT sequence numbering” or “numbered according to IMTG,” refers to numbering of the sequence encoding a variable region according to the IMGT. For the heavy chain variable domain, when numbered according to IMGT, the hypervariable region ranges from amino acid positions 27 to 38 for CDR1, amino acid positions 56 to 65 for CDR2, and amino acid positions 105 to 117 for CDR3. For the light chain variable domain, when numbered according to IMGT, the hypervariable region ranges from amino acid positions 27 to 38 for CDR1, amino acid positions 56 to 65 for CDR2, and amino acid positions 105 to 117 for CDR3.

In some embodiments, the cargo or payload may comprise or may encode antibodies which have been produced using methods known in the art such as, but are not limited to immunization and display technologies (e.g., phage display, yeast display, and ribosomal display), hybridoma technology, heavy and light chain variable region cDNA sequences selected from hybridomas or from other sources.

In some embodiments, the cargo or payload may comprise or may encode antibodies which were developed using any naturally occurring or synthetic antigen. As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen. As used herein, “antigens” also refer to binding partners for specific antibodies or binding agents in a display library.

As used herein, the term “monoclonal antibody” refers to an antibody obtained from a population of substantially homogeneous cells (or clones), e.g., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity.

In some embodiments, the cargo or payload may comprise or may encode antibody mimetics. As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold. In some embodiments, antibody mimetics may be those known in the art including, but are not limited to affibody molecules, affilins, affitins, anticalins, avimers, Centyrins, DARPINS™, fynomers, Kunitz domains, and domain peptides. In other embodiments, antibody mimetics may include one or more non-peptide regions.

i. Antibody Fragments and Variants

In some embodiments, the cargo or payload may comprise or may encode antibody fragments which comprise antigen binding regions from full-length antibodies. Non-limiting examples of antibody fragments include Fab, Fab′, F(ab′)2, and Fv fragments, diabodies, linear antibodies, single-chain antibody molecules, and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)2 fragment that has two antigen-binding sites and is still capable of cross-linking antigen. Compounds and/or compositions of the present invention may comprise one or more of these fragments.

In some embodiments, the Fc region may be a modified Fc region wherein the Fc region may have a single amino acid substitution as compared to the corresponding sequence for the wild-type Fc region, wherein the single amino acid substitution yields an Fc region with preferred properties to those of the wild-type Fc region. Non-limiting examples of Fc properties that may be altered by the single amino acid substitution include binding properties or response to pH conditions.

As used herein, the term “Fv” refers to an antibody fragment comprising the minimum fragment on an antibody needed to form a complete antigen binding site. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a flexible linker between the light chain variable domain and the heavy chain variable domain to form a single chain Fv (scFv) or through the introduction of a disulfide bridge between heavy and light chain variable domains.

As used herein, the term “single chain Fv” or “scFv” refers to a fusion protein of V_{H} and V_{L} antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition, or structure as compared to a native antibody. Antibody variants may include, but are not limited to, antibodies with altered isotypes (e.g., IgA, IgD, IgE, IgG₁, IgG₂, IgG₃, IgG₄, or IgM), humanized variants, optimized variants, multispecific antibody variants (e.g., bispecific variants), and antibody fragments.

ii. Multispecific Antibodies

In some embodiments, the cargo or payload may be or may encode antibodies that bind more than one epitope. As used herein, the terms “multibody” or “multispecific antibody” refer to an antibody wherein two or more variable regions bind to different epitopes. The epitopes may be on the same or different targets. In certain embodiments, a multispecific antibody is a “bispecific antibody,” which recognizes two different epitopes on the same or different antigens.

In some embodiments, multi-specific antibodies may be prepared by the methods used by BIOATLA® and described in International Patent publication WO201109726, the contents of which are herein incorporated by reference in their entirety. First a library of homologous, naturally occurring antibodies is generated by any method known in the art (e.g., mammalian cell surface display), then screened by flow cytometry (e.g., FACSARIA™) or another screening method, for multi-specific antibodies that specifically bind to two or more target antigens. In some embodiments, the identified multi-specific antibodies are further evolved by any method known in the art, to produce a set of modified multi-specific antibodies. These modified multi-specific antibodies are screened for binding to the target antigens. In some embodiments, the multi-specific antibody may be further optimized by screening the evolved modified multi-specific antibodies for optimized or desired characteristics.

In some embodiments, multi-specific antibodies may be prepared by the methods used by BIOATLA® and described in Unites States Publication No. US20150252119, the contents of which are herein incorporated by reference in their entirety. In one approach, the variable domains of two parent antibodies, wherein the parent antibodies are monoclonal antibodies are evolved using any method known in the art in a manner that allows a single light chain to functionally complement heavy chains of two different parent antibodies. Another approach requires evolving the heavy chain of a single parent antibody to recognize a second target antigen. A third approach involves evolving the light chain of a parent antibody so as to recognize a second target antigen. Methods for polypeptide evolution are described in International Publication WO2012009026, the contents of which are herein incorporated by reference in their entirety, and include as non-limiting examples, Comprehensive Positional Evolution (CPE), Combinatorial Protein Synthesis (CPS), Comprehensive Positional Insertion (CPI), Comprehensive Positional Deletion (CPD), or any combination thereof. The Fc region of the multi-specific antibodies described in United States Publication No. US20150252119 may be created using a knob-in-hole approach, or any other method that allows the Fc domain to form heterodimers. The resultant multi-specific antibodies may be further evolved for improved characteristics or properties such as binding affinity for the target antigen.

iii. Bispecific Antibodies

In some embodiments, the cargo or payload may be or may encode bispecific antibodies. As used herein, the term “bispecific antibody” refers to an antibody capable of binding two different antigens. Such antibodies typically comprise regions from at least two different antibodies. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen.

In some cases, the cargo or payload may be or may encode bispecific antibodies comprising antigen-binding regions from two different anti-tau antibodies. For example, such bispecific antibodies may comprise binding regions from two different antibodies

Bispecific antibody frameworks may include any of those described in Riethmuller, G., 2012. Cancer Immunity. 12:12-18; Marvin, J. S. et al., 2005. Acta Pharmacologica Sinica. 26(6):649-58; and Schaefer, W. et al., 2011. PNAS. 108(27):11187-92, the contents of each of which are herein incorporated by reference in their entirety.

New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

Of the two paratopes that form the tops of the variable domains of a bispecific antibody, one can be directed against a target antigen and the other against a T-lymphocyte antigen like CD3. In the case of trifunctional antibodies, the Fc region may additionally bind to a cell that expresses Fc receptors, like a macrophage, a natural killer (NK) cell or a dendritic cell. In sum, the targeted cell is connected to one or two cells of the immune system, which subsequently destroy it.

Other types of bispecific antibodies have been designed to overcome certain problems, such as short half-life, immunogenicity and side-effects caused by cytokine liberation. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bi-specific T-cell engagers (BiTEs) and mAb2's, antibodies engineered to contain an Fcab antigen-binding fragment instead of the Fc constant region.

Using molecular genetics, two scFvs can be engineered in tandem into a single polypeptide, separated by a linker domain, called a “tandem scFv” (tascFv). TascFvs have been found to be poorly soluble and require refolding when produced in bacteria, or they may be manufactured in mammalian cell culture systems, which avoids refolding requirements but may result in poor yields. Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications, and are described as “Bispecific T-cell Engagers (BiTE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed.

In some embodiments, the cargo or payload may be or may encode antibodies comprising a single antigen-binding domain. These molecules are extremely small, with molecular weights approximately one-tenth of those observed for full-sized mAbs. Further antibodies may include “nanobodies” derived from the antigen-binding variable heavy chain regions (VHHS) of heavy chain antibodies found in camels and llamas, which lack light chains.

Disclosed and claimed in PCT Publication WO2014144573 (the contents of which are herein incorporated by reference in its entirety) to Memorial Sloan-Kettering Cancer Center are multimerization technologies for making dimeric multispecific binding agents (e.g., fusion proteins comprising antibody components) with improved properties over multispecific binding agents without the capability of dimerization.

In some cases, the cargo or payload may be or may encode tetravalent bispecific antibodies (TetBiAbs as disclosed and claimed in PCT Publication WO2014144357, the contents of which are herein incorporated in its entirety). TetBiAbs feature a second pair of Fab fragments with a second antigen specificity attached to the C-terminus of an antibody, thus providing a molecule that is bivalent for each of the two antigen specificities. The tetravalent antibody is produced by genetic engineering methods, by linking an antibody heavy chain covalently to a Fab light chain, which associates with its cognate, co-expressed Fab heavy chain.

In some aspects, the cargo or payload may be or may encode biosynthetic antibodies as described in U.S. Pat. No. 5,091,513 (the contents of which are herein incorporated by reference in their entirety). Such an antibody may include one or more sequences of amino acids constituting a region which behaves as a biosynthetic antibody binding site (BABS). The sites comprise 1) non-covalently associated or disulfide bonded synthetic V_{H} and V_{L} dimers, 2) V_{H}-V_{L} or V_{L}-V_{H} single chains wherein the V_{H} and V_{L} are attached by a polypeptide linker, or 3) individuals V_{H} or V_{L} domains. The binding domains comprise linked CDR and FR regions, which may be derived from separate immunoglobulins. The biosynthetic antibodies may also include other polypeptide sequences which function, e.g., as an enzyme, toxin, binding site, or site of attachment to an immobilization media or radioactive atom. Methods are disclosed for producing the biosynthetic antibodies, for designing BABS having any specificity that can be elicited by in vivo generation of antibody, and for producing analogs thereof.

In some embodiments, the cargo or payload may be or may encode antibodies with antibody acceptor frameworks taught in U.S. Pat. No. 8,399,625. Such antibody acceptor frameworks may be particularly well suited accepting CDRs from an antibody of interest. In some cases, CDRs from anti-tau antibodies known in the art or developed according to the methods presented herein may be used.

iv. Miniaturized Antibody

In some embodiments, the cargo or payload may be or may encode a “miniaturized” antibody. Among the best examples of mAb miniaturization are the small modular immunopharmaceuticals (SMIPs) from Trubion Pharmaceuticals. These molecules, which can be monovalent or bivalent, are recombinant single-chain molecules containing one V_{L}, one V_{H} antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythrematosus (SLE) and B cell lymphomas were ultimately discontinued. Trubion and Facet Biotechnology are collaborating in the development of TRU-016, an anti-CD37 SMIP, for the treatment of CLL and other lymphoid neoplasias, a project that has reached Phase 2. Wyeth has licensed the anti-CD20 SMIP SBI-087 for the treatment of autoimmune diseases, including RA, SLE, and possibly multiple sclerosis, although these projects remain in the earliest stages of clinical testing.

v. Diabodies

In some embodiments, the cargo or payload may be or may encode diabodies. As used herein, the term “diabody” refers to a small antibody fragment with two antigen-binding sites. Diabodies comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites.

Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al, Proc. Natl. Acad. Sci., 92: 7021-7025, 1995). Few diabodies have entered clinical development. An iodine-123-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials.gov NCT00647153).

vi. Unibody

In some embodiments, the cargo or payload may be or may encode a “unibody,” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promote intracellular signaling complex formation. These contentions are, however, largely supported by laboratory, rather than clinical, evidence. Other antibodies may be “miniaturized” antibodies, which are compacted 100 kDa antibodies.

vii. Intrabodies

In some embodiments, the cargo or payload may be or may encode intrabodies. The term “intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling, and cell division. In some embodiments, methods of the present invention may include intrabody-based therapies. In some such embodiments, variable domain sequences and/or CDR sequences disclosed herein may be incorporated into one or more constructs for intrabody-based therapy. For example, intrabodies may target one or more glycated intracellular proteins or may modulate the interaction between one or more glycated intracellular proteins and an alternative protein.

More than two decades ago, intracellular antibodies against intracellular targets were first described (Biocca, Neuberger and Cattaneo EMBO J. 9: 101-108, 1990, the contents of which are herein incorporated by reference in their entirety). The intracellular expression of intrabodies in different compartments of mammalian cells allows blocking or modulation of the function of endogenous molecules (Biocca, et al., EMBO J. 9: 101-108, 1990; Colby et al., Proc. Natl. Acad. Sci. U.S.A. 101: 17616-21, 2004, the contents of which are herein incorporated by reference in their entirety). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. They have been largely employed as research tools and are emerging as therapeutic molecules for the treatment of human diseases such as viral pathologies, cancer and misfolding diseases. The fast-growing bio-market of recombinant antibodies provides intrabodies with enhanced binding specificity, stability, and solubility, together with lower immunogenicity, for their use in therapy.

In some embodiments, intrabodies have advantages over interfering RNA (iRNA); for example, iRNA has been shown to exert multiple non-specific effects, whereas intrabodies have been shown to have high specificity and affinity to target antigens. Furthermore, as proteins, intrabodies possess a much longer active half-life than iRNA. Thus, when the active half-life of the intracellular target molecule is long, gene silencing through iRNA may be slow to yield an effect, whereas the effects of intrabody expression can be almost instantaneous. Lastly, it is possible to design intrabodies to block certain binding interactions of a particular target molecule, while sparing others.

Intrabodies are often single chain variable fragments (scFvs) expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies may be produced for use in the viral genomes of the invention using methods known in the art, such as those disclosed and reviewed in: Marasco et al., 1993 Proc. Natl. Acad. Sci. USA, 90: 7889-7893; Chen et al., 1994, Hum. Gene Ther. 5:595-601; Chen et al., 1994, Proc. Natl. Acad. Sci. USA, 91: 5932-5936; Maciejewski et al., 1995, Nature Med., 1: 667-673; Marasco, 1995, Immunotech, 1: 1-19; Mhashilkar, et al., 1995, EMBO J. 14: 1542-51; Chen et al., 1996, Hum. Gene Therap., 7: 1515-1525; Marasco, Gene Ther. 4:11-15, 1997; Rondon and Marasco, 1997, Annu. Rev. Microbiol. 51:257-283; Cohen, et al., 1998, Oncogene 17:2445-56; Proba et al., 1998, J. Mol. Biol. 275:245-253; Cohen et al., 1998, Oncogene 17:2445-2456; Hassanzadeh, et al., 1998, FEBSLett. 437:81-6; Richardson et al., 1998, Gene Ther. 5:635-44; Ohage and Steipe, 1999, J. Mol. Biol. 291:1119-1128; Ohage et al., 1999, J. Mol. Biol. 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J. Biol. Chem. 277:45075-85; Mhashilkar et al., 2002, Gene Ther. 9:307-19; and Wheeler et al., 2003, FASEB J. 17: 1733-5; and references cited therein. In particular, a CCR5 intrabody has been produced by Steinberger et al., 2000, Proc. Natl. Acad. Sci. USA 97:805-810. See generally Marasco, W A, 1998, “Intrabodies: Basic Research and Clinical Gene Therapy Applications” Springer: New York; and for a review of scFvs, see Pluckthun in “The Pharmacology of Monoclonal Antibodies,” 1994, vol. 113, Rosenburg and Moore eds. Springer-Verlag, New York, pp. 269-315; the contents of each of which are each incorporated by reference in their entireties.

Sequences from donor antibodies may be used to develop intrabodies. Intrabodies are often recombinantly expressed as single domain fragments such as isolated V_{H} and V_{L} domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and are capable of modulating the expression or activity of target genes through their specific binding activity. Single chain antibodies can also be expressed as a single chain variable region fragment joined to the light chain constant region.

As is known in the art, an intrabody can be engineered into recombinant polynucleotide vectors to encode sub-cellular trafficking signals at its N or C terminus to allow expression at high concentrations in the sub-cellular compartments where a target protein is located. For example, intrabodies targeted to the endoplasmic reticulum (ER) are engineered to incorporate a leader peptide and, optionally, a C-terminal ER retention signal. Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

There are certain technical challenges with intrabody expression. In particular, protein conformational folding and structural stability of the newly-synthesized intrabody within the cell is affected by reducing conditions of the intracellular environment.

Intrabodies of the invention may be promising therapeutic agents for the treatment of misfolding diseases, including Tauopathies, prion diseases, Alzheimer's, Parkinson's, and Huntington's, because of their virtually infinite ability to specifically recognize the different conformations of a protein, including pathological isoforms, and because they can be targeted to the potential sites of aggregation (both intra- and extracellular sites). These molecules can work as neutralizing agents against amyloidogenic proteins by preventing their aggregation, and/or as molecular shunters of intracellular traffic by rerouting the protein from its potential aggregation site.

viii. Maxibodies

In some embodiments, the cargo or payload may be or may encode a maxibody (bivalent scFV fused to the amino terminus of the Fc (CH2-CH3 domains) of IgG.

c. Chimeric Antigen Receptors (CARs)

In some embodiments, the cargo or payload may be or may encode a chimeric antigen receptor (CAR) which when transduced into immune cells (e.g., T cells and NK cells), can re-direct the immune cells against the target (e.g., a tumor cell) which expresses a molecule recognized by the extracellular target moiety of the CAR.

As used herein, the term “chimeric antigen receptor (CAR)” refers to a synthetic receptor that mimics TCR on the surface of T cells. In general, a CAR is composed of an extracellular targeting domain, a transmembrane domain/region and an intracellular signaling/activation domain. In a standard CAR receptor, the components: the extracellular targeting domain, transmembrane domain and intracellular signaling/activation domain, are linearly constructed as a single fusion protein. The extracellular region comprises a targeting domain/moiety (e.g., a scFv) that recognizes a specific tumor antigen or other tumor cell-surface molecules. The intracellular region may contain a signaling domain of TCR complex (e.g., the signal region of CD3ζ), and/or one or more costimulatory signaling domains, such as those from CD28, 4-1BB (CD137) and OX-40 (CD134). For example, a “first-generation CAR” only has the CD3ζ signaling domain, whereas in an effort to augment T-cell persistence and proliferation, costimulatory intracellular domains are added, giving rise to second generation CARs having a CD3ζ signal domain plus one costimulatory signaling domain, and third generation CARs having CD3ζ signal domain plus two or more costimulatory signaling domains. A CAR, when expressed by a T cell, endows the T cell with antigen specificity determined by the extracellular targeting moiety of the CAR. In some aspects one could add one or more elements such as homing and suicide genes to develop a more competent and safer architecture of CAR (so called the fourth generation CAR).

In some embodiments, the extracellular targeting domain is joined through the hinge (also called space domain or spacer) and transmembrane regions to an intracellular signaling domain. The hinge connects the extracellular targeting domain to the transmembrane domain which transverses the cell membrane and connects to the intracellular signaling domain. The hinge may need to be varied to optimize the potency of CAR transformed cells toward cancer cells due to the size of the target protein where the targeting moiety binds, and the size and affinity of the targeting domain itself. Upon recognition and binding of the targeting moiety to the target cell, the intracellular signaling domain leads to an activation signal to the CAR T cell, which is further amplified by the “second signal” from one or more intracellular costimulatory domains. The CAR T cell, once activated, can destroy the target cell.

In some embodiments, the CAR may be split into two parts, each part is linked a dimerizing domain, such that an input that triggers the dimerization promotes assembly of the intact functional receptor. Wu and Lim reported a split CAR in which the extracellular CD19 binding domain and the intracellular signaling element are separated and linked to the FKBP domain and the FRB* (T2089L mutant of FKBP-rapamycin binding) domain that heterodimerize in the presence of the rapamycin analog AP21967. The split receptor is assembled in the presence of AP21967 and together with the specific antigen binding, activates T cells (Wu et al., Science, 2015, 625(6258): aab4077, the contents of which are herein incorporated by reference in its entirety).

In some embodiments, the CAR may be designed as an inducible CAR which has an incorporation of a Tet-On inducible system to a CD19 CAR construct. The CD19 CAR is activated only in the presence of doxycycline (Dox). Sakemura reported that Tet-CD19CAR T cells in the presence of Dox were equivalently cytotoxic against CD19+ cell lines and had equivalent cytokine production and proliferation upon CD19 stimulation, compared with conventional CD19CAR T cells (Sakemura et al., Cancer Immuno. Res., 2016, Jun. 21, Epub; the contents of which is herein incorporated by reference in its entirety). The dual systems provide more flexibility to turn-on and off of the CAR expression in transduced T cells.

In some embodiments, the cargo or payload may be or may encode a first generation CAR, or a second generation CAR, or a third generation CAR, or a fourth generation CAR. In some embodiments, the cargo or payload may be or may encode a full CAR construct composed of the extracellular domain, the hinge and transmembrane domain and the intracellular signaling region. In other embodiments, the cargo or payload may be or may encode a component of the full CAR construct including an extracellular targeting moiety, a hinge region, a transmembrane domain, an intracellular signaling domain, one or more co-stimulatory domain, and other additional elements that improve CAR architecture and functionality including but not limited to a leader sequence, a homing element and a safety switch, or the combination of such components.

In some embodiments, the cargo or payload may be or may encode a tunable CARs. The reversible on-off switch mechanism allows management of acute toxicity caused by excessive CAR-T cell expansion. The ligand conferred regulation of the CAR may be effective in offsetting tumor escape induced by antigen loss, avoiding functional exhaustion caused by tonic signaling due to chronic antigen exposure and improving the persistence of CAR expressing cells in vivo. The tunable CAR may be utilized to down regulate CAR expression to limit on target on tissue toxicity caused by tumor lysis syndrome. Down regulating the expression of the CARs following anti-tumor efficacy may prevent (1) on target off tumor toxicity caused by antigen expression in normal tissue, (2) antigen independent activation in vivo.

i. Extracellular Targeting Domain/Moiety

In some embodiments, the extracellular target moiety of a CAR may be any agent that recognizes and binds to a given target molecule, for example, a neoantigen on tumor cells, with high specificity and affinity. The target moiety may be an antibody and variants thereof that specifically binds to a target molecule on tumor cells, or a peptide aptamer selected from a random sequence pool based on its ability to bind to the target molecule on tumor cells, or a variant or fragment thereof that can bind to the target molecule on tumor cells, or an antigen recognition domain from native T-cell receptor (TCR) (e.g. CD4 extracellular domain to recognize HIV infected cells), or exotic recognition components such as a linked cytokine that leads to recognition of target cells bearing the cytokine receptor, or a natural ligand of a receptor.

In some embodiments, the targeting domain of a CAR may be a Ig NAR, a Fab fragment, a Fab′ fragment, a F(ab)′2 fragment, a F(ab)′3 fragment, Fv, a single chain variable fragment (scFv), a bis-scFv, a (scFv)2, a minibody, a diabody, a triabody, a tetrabody, a disulfide stabilized Fv protein (dsFv), a unibody, a nanobody, or an antigen binding region derived from an antibody that specifically recognizes a target molecule, for example a tumor specific antigen (TSA). In one embodiment, the targeting moiety is a scFv antibody. The scFv domain, when it is expressed on the surface of a CAR T cell and subsequently binds to a target protein on a cancer cell, is able to maintain the CAR T cell in proximity to the cancer cell and to trigger the activation of the T cell. A scFv can be generated using routine recombinant DNA technology techniques and is discussed in the present invention.

In some embodiments, the targeting moiety of a CAR construct may be an aptamer such as a peptide aptamer that specifically binds to a target molecule of interest. The peptide aptamer may be selected from a random sequence pool based on its ability to bind to the target molecule of interest.

In some embodiments, the targeting moiety of a CAR construct may be a natural ligand of the target molecule, or a variant and/or fragment thereof capable of binding the target molecule. In some aspects, the targeting moiety of a CAR may be a receptor of the target molecule, for example, a full length human CD27, as a CD70 receptor, may be fused in frame to the signaling domain of CD3 (forming a CD27 chimeric receptor as an immunotherapeutic agent for CD70-positive malignancies.

In some embodiments, the targeting moiety of a CAR may recognize a tumor specific antigen (TSA), for example a cancer neoantigen which is restrictedly expressed on tumor cells.

As non-limiting examples, the CAR of the present invention may comprise the extracellular targeting domain capable of binding to a tumor specific antigen selected from 5T4, 707-AP, A33, AFP (α-fetoprotein), AKAP-4 (A kinase anchor protein 4), ALK, α5β1-integrin, androgen receptor, annexin II, alpha-actinin-4, ART-4, B1, B7H3, B7H4, BAGE (B melanoma antigen), BCMA, BCR-ABL fusion protein, beta-catenin, BKT-antigen, BTAA, CA-I (carbonic anhydrase I), CA50 (cancer antigen 50), CA125, CA15-3, CA195, CA242, calretinin, CAIX (carbonic anhydrase), CAMEL (cytotoxic T-lymphocyte recognized antigen on melanoma), CAM43, CAP-1, Caspase-8/m, CD4, CD5, CD7, CD19, CD20, CD22, CD23, CD25, CD27/m, CD28, CD30, CD33, CD34, CD36, CD38, CD40/CD154, CD41, CD44v6, CD44v7/8, CD45,CD49f, CD56, CD68KP1, CD74, CD79a/CD79b, CD103, CD123, CD133, CD138, CD171, cdc27/m, CDK4 (cyclin dependent kinase 4), CDKN2A, CDS, CEA (carcinoembryonic antigen), CEACAM5, CEACAM6, chromogranin, c-Met, c-Myc, coa-1, CSAp, CT7, CT10, cyclophilin B, cyclin B1, cytoplasmic tyrosine kinases, cytokeratin, DAM-10, DAM-6, dek-can fusion protein, desmin, DEPDCl (DEP domain containing 1), E2A-PRL, EBNA, EGF-R (epidermal growth factor receptor), EGP-1(epithelial glycoprotein-1) (TROP-2), EGP-2, EGP-40, EGFR (epidermal growth factor receptor), EGFRvIII, EF-2, ELF2M, EMMPRIN, EpCAM (epithelial cell adhesion molecule), EphA2, Epstein Barr virus antigens, Erb (ErbB1; ErbB3; ErbB4), ETA (epithelial tumor antigen), ETV6-AML1 fusion protein, FAP (fibroblast activation protein), FBP (folate-binding protein), FGF-5, folate receptor, FOS related antigen 1, fucosyl GM1, G250, GAGE (GAGE-1; GAGE-2), galectin, GD2 (ganglioside), GD3, GFAP (glial fibrillary acidic protein), GM2 (oncofetal antigen-immunogenic-1; OFA-I-1), GnT-V, Gp100, H4-RET, HAGE (helicase antigen), HER-2/neu, HIFs (hypoxia inducible factors), HIF-1, HIF-2, HLA-A2, HLA-A*0201-R170I, HLA-A11, HMWMAA, Hom/Mel-40, HSP70-2M (Heat shock protein 70), HST-2, HTgp-175, hTERT (or hTRT), human papillomavirus-E6/human papillomavirus-E7 and E6, iCE (immune-capture EIA), IGF-1R, IGH-IGK, IL-2R, IL-5, ILK (integrin-linked kinase), IMP3 (insulin-like growth factor II mRNA-binding protein 3), IRF4 (interferon regulatory factor 4), KDR (kinase insert domain receptor), KIAA0205, KRAB-zinc finger protein (KID)-3; KID31, KSA (17-1A), K-ras, LAGE, LCK, LDLR/FUT (LDLR-fucosyltransferaseAS fusion protein), LeY (Lewis Y), MAD-CT-1, MAGE (tyrosinase, melanoma-associated antigen) (MAGE-1; MAGE-3), melan-A tumor antigen (MART), MART-2/Ski, MC1R (melanocortin 1 receptor), MDM2, mesothelin, MPHOSPHI, MSA(muscle-specific actin), mTOR (mammalian targets of rapamycin), MUC-1, MUC-2, MUM-1 (melanoma associated antigen (mutated) 1), MUM-2, MUM-3, Myosin/m, MYL-RAR, NA88-A, N-acetylglucosaminyltransferase, neo-PAP, NF-KB (nuclear factor-kappa B), neurofilament, NSE (neuron-specific enolase), Notch receptors, NuMa, N-Ras, NY-BR-1, NY—CO-1, NY-ESO-1, Oncostatin M, OS-9, OY-TES1, p53 mutants, p190 minor bcr-abl, p15(58), pl85erbB2, pl80erbB-3, PAGE (prostate associated gene), PAP (prostatic acid phosphatase), PAX3, PAX5, PDGFR (platelet derived growth factor receptor), cytochrome P450 involved in piperidine and pyrrolidine utilization (PIPA), Pml-RAR alpha fusion protein, PR-3 (proteinase 3), PSA (prostate specific antigen), PSM, PSMA (Prostate stem cell antigen), PRAME (preferentially expressed antigen of melanoma), PTPRK, RAGE (renal tumor antigen), Raf (A-Raf, B-Raf and C-Raf), Ras, receptor tyrosine kinases, RCAS1, RGSS, ROR1 (receptor tyrosine kinase-like orphan receptor 1), RUl, RU2, SAGE, SART-1, SART-3, SCP-1, SDCCAG16, SP-17 (sperm protein 17), src-family, SSX (synovial sarcoma X breakpoint)-1, SSX-2(HOM-MEL-40), SSX-3, SSX-4, SSX-5, STAT-3, STAT-5, STAT-6, STEAD, STn, survivin, syk-ZAP70, TA-90 (Mac-2 binding proteincyclophilin C-associated protein), TAAL6, TACSTD1 (tumor associated calcium signal transducer 1), TACSTD2, TAG-72-4, TAGE, TARP (T cell receptor gamma alternate reading frame protein), TEL/AML1 fusion protein, TEM1, TEM8 (endosialin or CD248), TGFO, TIE2, TLP, TMPRSS2 ETS fusion gene, TNF-receptor (TNF-α receptor, TNF-β receptor; or TNF-γ receptor), transferrin receptor, TPS, TRP-1 (tyrosine related protein 1), TRP-2, TRP-2/INT2, TSP-180, VEGF receptor, WNT, WT-1 (Wilm's tumor antigen) and XAGE.

In some embodiments, the cargo or payload may be or may encode a CAR which comprises a universal immune receptor which has a targeting moiety capable of binding to a labelled antigen.

In some embodiments, the cargo or payload may be or may encode a CAR which comprises a targeting moiety capable of binding to a pathogen antigen.

In some embodiments, the cargo or payload may be or may encode a CAR which comprises a targeting moiety capable of binding to non-protein molecules such as tumor-associated glycolipids and carbohydrates.

In some embodiments, the cargo or payload may be or may encode a CAR which comprises a targeting moiety capable of binding to a component within the tumor microenvironment including proteins expressed in various tumor stroma cells including tumor associated macrophages (TAMs), immature monocytes, immature dendritic cells, immunosuppressive CD4+CD25+ regulatory T cells (Treg) and MDSCs.

In some embodiments, the cargo or payload may be or may encode a CAR which comprises a targeting moiety capable of binding to a cell surface adhesion molecule, a surface molecule of an inflammatory cell that appears in an autoimmune disease, or a TCR causing autoimmunity. As non-limiting examples, the targeting moiety of the present invention may be a scFv antibody that recognizes a tumor specific antigen (TSA), for example scFvs of antibodies SS, SS1 and HN1 that specifically recognize and bind to human mesothelin, scFv of antibody of GD2, a CD19 antigen binding domain, a NKG2D ligand binding domain, human anti-mesothelin scFvs, an anti-CS1 binding agent, an anti-BCMA binding domain, anti-CD19 scFv antibody, GFR alpha 4 antigen binding fragments, anti-CLL-1 (C-type lectin-like molecule 1) binding domains, CD33 binding domains, a GPC3 (glypican-3) binding domain, a GFR alpha4 (Glycosyl-phosphatidylinositol (GPI)-linked GDNF family a-receptor 4 cell-surface receptor) binding domain, CD123 binding domains, an anti-ROR1 antibody or fragments thereof, scFvs specific to GPC-3, scFv for CSPG4, and scFv for folate receptor alpha.

ii. Intracellular Signaling Domains

The intracellular domain of a CAR fusion polypeptide, after binding to its target molecule, transmits a signal to the immune effector cell, activating at least one of the normal effector functions of immune effector cells, including cytolytic activity (e.g., cytokine secretion) or helper activity. Therefore, the intracellular domain comprises an “intracellular signaling domain” of a T cell receptor (TCR).

In some aspects, the entire intracellular signaling domain can be employed. In other aspects, a truncated portion of the intracellular signaling domain may be used in place of the intact chain as long as it transduces the effector function signal.

In some embodiments, the intracellular signaling domain may contain signaling motifs which are known as immunoreceptor tyrosine-based activation motifs (ITAMs). Examples of ITAM containing cytoplasmic signaling sequences include those derived from TCR CD3zeta, FcR gamma, FcR beta, CD3 gamma, CD3 delta, CD3 epsilon, CD5, CD22, CD79a, CD79b, and CD66d. In one example, the intracellular signaling domain is a CD3 zeta (CD3ζ) signaling domain.

In some embodiments, the intracellular region further comprises one or more costimulatory signaling domains which provide additional signals to the immune effector cells. These costimulatory signaling domains, in combination with the signaling domain can further improve expansion, activation, memory, persistence, and tumor-eradicating efficiency of CAR engineered immune cells (e.g., CAR T cells). In some cases, the costimulatory signaling region contains 1, 2, 3, or 4 cytoplasmic domains of one or more intracellular signaling and/or costimulatory molecules. The costimulatory signaling domain may be the intracellular/cytoplasmic domain of a costimulatory molecule, including but not limited to CD2, CD7, CD27, CD28, 4-1BB (CD137), OX40 (CD134), CD30, CD40, ICOS (CD278), GITR (glucocorticoid-induced tumor necrosis factor receptor), LFA-1 (lymphocyte function-associated antigen-1), LIGHT, NKG2C, B7-H3. In one example, the costimulatory signaling domain is derived from the cytoplasmic domain of CD28. In another example, the costimulatory signaling domain is derived from the cytoplasmic domain of 4-1BB (CD137). In another example, the co-stimulatory signaling domain may be an intracellular domain of GITR as taught in U.S. Pat. No. 9,175,308; the contents of which are incorporated herein by reference in its entirety.

In some embodiments, the intracellular region may comprise a functional signaling domain from a protein selected from the group consisting of an MHC class I molecule, a TNF receptor protein, an immunoglobulin-like protein, a cytokine receptor, an integrin, a signaling lymphocytic activation protein (SLAM) such as CD48, CD229, 2B4, CD84, NTB-A, CRACC, BLAME,CD2F-10, SLAMF6, SLAMF7, an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), SLAMF7, NKp80 (KLRF1), NKp44, NKp30, NKp46, CD19, CD4, CD8alpha, CD8beta, IL2R beta, IL2R gamma, IL7R alpha, IL-15Ra, ITGA4, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD1 Ib, ITGAX, CD1 Ic, ITGB1, CD29, ITGB2, CD18, LFA-1, ITGB7, NKG2D, NKG2C, NKD2C SLP76, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, CD270 (HVEM), GADS, SLP-76, PAG/Cbp, CD19a, a ligand that specifically binds with CD83, DAP 10, TRIM, ZAP70, Killer immunoglobulin receptors (KIRs) such as KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, KIR3DL3, and KIR2DP1; lectin related NK cell receptors such as Ly49, Ly49A, and Ly49C.

In some embodiments, the intracellular signaling domain of the present invention may contain signaling domains derived from JAK-STAT. In other embodiments, the intracellular signaling domain of the present invention may contain signaling domains derived from DAP-12 (Death associated protein 12) (Topfer et al., Immunol., 2015, 194: 3201-3212; and Wang et al., Cancer Immunol., 2015, 3: 815-826). DAP-12 is a key signal transduction receptor in NK cells. The activating signals mediated by DAP-12 play important roles in triggering NK cell cytotoxicity responses toward certain tumor cells and virally infected cells. The cytoplasmic domain of DAP12 contains an Immunoreceptor Tyrosine-based Activation Motif (ITAM). Accordingly, a CAR containing a DAP12-derived signaling domain may be used for adoptive transfer of NK cells.

iii. Transmembrane Domains

In some embodiments, the CAR may comprise a transmembrane domain. As used herein, the term “Transmembrane domain (TM)” refers broadly to an amino acid sequence of about 15 residues in length which spans the plasma membrane. The transmembrane domain may include at least 20, 21, 22, 23, 24, 25, 26, 27, 28, 29, 30, 31, 32, 33, 34, 35, 36, 37, 38, 39, 40, 41, 42, 43, 44, or 45 amino acid residues and spans the plasma membrane. In some embodiments, the transmembrane domain may be derived either from a natural or from a synthetic source. The transmembrane domain of a CAR may be derived from any naturally membrane-bound or transmembrane protein. For example, the transmembrane region may be derived from (e.g., comprise at least the transmembrane region(s) of) the alpha, beta or zeta chain of the T-cell receptor, CD3 epsilon, CD4, CD5, CD8, CD8a, CD9, CD16, CD22, CD33, CD28, CD37, CD45, CD64, CD80, CD86, CD134, CD137, CD152, or CD154.

Alternatively, the transmembrane domain of the present invention may be synthetic. In some aspects, the synthetic sequence may comprise predominantly hydrophobic residues such as leucine and valine.

In some embodiments, the transmembrane domain may be selected from the group consisting of a CD8a transmembrane domain, a CD4 transmembrane domain, a CD 28 transmembrane domain, a CTLA-4 transmembrane domain, a PD-1 transmembrane domain, and a human IgG4 Fc region.

In some embodiments, the CAR may comprise an optional hinge region (also called spacer). A hinge sequence is a short sequence of amino acids that facilitates flexibility of the extracellular targeting domain that moves the target binding domain away from the effector cell surface to enable proper cell/cell contact, target binding and effector cell activation. The hinge sequence may be positioned between the targeting moiety and the transmembrane domain. The hinge sequence can be any suitable sequence derived or obtained from any suitable molecule. The hinge sequence may be derived from all or part of an immunoglobulin (e.g., IgG1, IgG2, IgG3, IgG4) hinge region, e.g., the sequence that falls between the CHI and CH2 domains of an immunoglobulin, e.g., an IgG4 Fc hinge, the extracellular regions of type 1 membrane proteins such as CD8a CD4, CD28 and CD7, which may be a wild-type sequence or a derivative. Some hinge regions include an immunoglobulin CH3 domain or both a CH3 domain and a CH2 domain. In certain embodiments, the hinge region may be modified from an IgG1, IgG2, IgG3, or IgG4 that includes one or more amino acid residues, for example, 1, 2, 3, 4 or 5 residues, substituted with an amino acid residue different from that present in an unmodified hinge.

In some embodiments, the CAR may comprise one or more linkers between any of the domains of the CAR. The linker may be between 1-30 amino acids long. In this regard, the linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. In other embodiments, the linker may be flexible.

In some embodiments, the components including the targeting moiety, transmembrane domain and intracellular signaling domains may be constructed in a single fusion polypeptide. The fusion polypeptide may be the payload of an effector module of the invention.

In some embodiments, the cargo or payload may be or may encode a CD19 specific CAR targeting different B cell malignancies and HER2-specific CAR targeting sarcoma, glioblastoma, and advanced Her2-positive lung malignancy. Tandem CAR (TanCAR)

In some embodiments, the CAR may be a tandem chimeric antigen receptor (TanCAR) which is able to target two, three, four, or more tumor specific antigens. In some aspects, The CAR is a bispecific TanCAR including two targeting domains which recognize two different TSAs on tumor cells. The bispecific TanCAR may be further defined as comprising an extracellular region comprising a targeting domain (e.g., an antigen recognition domain) specific for a first tumor antigen and a targeting domain (e.g., an antigen recognition domain) specific for a second tumor antigen. In other aspects, the CAR is a multispecific TanCAR that includes three or more targeting domains configured in a tandem arrangement. The space between the targeting domains in the TanCAR may be between about 5 and about 30 amino acids in length, for example, 6, 7, 8, 9, 10, 11, 12, 13, 14, 15, 16, 17, 18, 19, 20, 21, 22, 23, 24, 25, 26, 27, 28, 29 and 30 amino acids.

iv. Split CAR

In some embodiments, the CAR components including the targeting moiety, transmembrane domain and intracellular signaling domains may be split into two or more parts such that it is dependent on multiple inputs that promote assembly of the intact functional receptor. As a non-limiting example, the split CAR consists of two parts that assemble in a small molecule-dependent manner; one part of the receptor features an extracellular antigen binding domain (e.g. scFv) and the other part has the intracellular signaling domains, such as the CD3ζ intracellular domain.

In other aspects, the split parts of the CAR system can be further modified to increase signal. As a non-limiting example, the second part of cytoplasmic fragment may be anchored to the plasma membrane by incorporating a transmembrane domain (e.g., CD8a transmembrane domain) to the construct. An additional extracellular domain may also be added to the second part of the CAR system, for instance an extracellular domain that mediates homo-dimerization. These modifications may increase receptor output activity, e.g., T cell activation.

In some embodiments, the two parts of the split CAR system contain heterodimerization domains that conditionally interact upon binding of a heterodimerizing small molecule. As such, the receptor components are assembled in the presence of the small molecule, to form an intact system which can then be activated by antigen engagement. Any known heterodimerizing components can be incorporated into a split CAR system. Other small molecule dependent heterodimerization domains may also be used, including, but not limited to, gibberellin-induced dimerization system (GID1-GAI), trimethoprim-SLF induced ecDHFR and FKBP dimerization and ABA (abscisic acid) induced dimerization of PP2C and PYL domains. The dual regulation using inducible assembly (e.g., ligand dependent dimerization) and degradation (e.g., destabilizing domain induced CAR degradation) of the split CAR system may provide more flexibility to control the activity of the CAR modified T cells.

v. Switchable CAR

In some embodiments, the CAR may be a switchable CAR which is a controllable CARs that can be transiently switched on in response to a stimulus (e.g. a small molecule). In this CAR design, a system is directly integrated in the hinge domain that separate the scFv domain from the cell membrane domain in the CAR. In a such system, it is possible to split or combine different key functions of a CAR such as activation and costimulation within different chains of a receptor complex, mimicking the complexity of the TCR native architecture. This integrated system can switch the scFv and antigen interaction between on/off states controlled by the absence/presence of the stimulus.

vi. Reversible CAR

In some embodiments, the CAR may be a reversible CAR system. In this CAR architecture, a LID domain (ligand-induced degradation) is incorporated into the CAR system. The CAR can be temporarily down-regulated by adding a ligand of the LID domain.

vii. Inhibitory CAR (iCAR)

In some embodiments, the CAR may be inhibitory CARs. Inhibitory CAR (iCAR) refers to a bispecific CAR design wherein a negative signal is used to enhance the tumor specificity and limit normal tissue toxicity. This design incorporates a second CAR having a surface antigen recognition domain combined with an inhibitory signal domain to limit T cell responsiveness even with concurrent engagement of an activating receptor. This antigen recognition domain is directed towards a normal tissue specific antigen such that the T cell can be activated in the presence of first target protein, but if the second protein that binds to the iCAR is present, the T cell activation is inhibited.

As a non-limiting example, iCARs against Prostate specific membrane antigen (PMSA) based on CTLA4 and PD1 inhibitory domains demonstrated the ability to selectively limit cytokine secretion, cytotoxicity and proliferation induced by T cell activation.

viii. Chimeric Switch Receptor

In some embodiments, the cargo or payload may be or may encode a chimeric switch receptors which can switch a negative signal to a positive signal. As used herein, the term “chimeric switch receptor” refers to a fusion protein comprising a first extracellular domain and a second transmembrane and intracellular domain, wherein the first domain includes a negative signal region and the second domain includes a positive intracellular signaling region. In some aspects, the fusion protein is a chimeric switch receptor that contains the extracellular domain of an inhibitory receptor on T cell fused to the transmembrane and cytoplasmic domain of a co-stimulatory receptor. This chimeric switch receptor may convert a T cell inhibitory signal into a T cell stimulatory signal.

As a non-limiting example, the chimeric switch receptor may comprise the extracellular domain of PD-1 fused to the transmembrane and cytoplasmic domain of CD28. In some aspects, extracellular domains of other inhibitory receptors such as CTLA-4, LAG-3, TIM-3, KIRs and BTLA may also be fused to the transmembrane and cytoplasmic domain derived from costimulatory receptors such as CD28, 4-1BB, CD27, OX40, CD40, GTIR and ICOS.

In some embodiments, chimeric switch receptors may include recombinant receptors comprising the extracellular cytokine-binding domain of an inhibitory cytokine receptor (e.g., IL-13 receptor α (IL-13Rα1), IL-10R, and IL-4Rα) fused to an intracellular signaling domain of a stimulatory cytokine receptor such as IL-2R (IL-2Rα, IL-2Rβ and IL-2Rgamma) and IL-7Ra. One example of such chimeric cytokine receptor is a recombinant receptor containing the cytokine-binding extracellular domain of IL-4Ra linked to the intracellular signaling domain of IL-7Ra.

In some embodiments, the chimeric switch receptor may be a chimeric TGFP receptor. The chimeric TGFP receptor may comprise an extracellular domain derived from a TGFP receptor such as TGFP receptor 1, TGFP receptor 2, TGFP receptor 3, or any other TGFP receptor or variant thereof, and a non-TGFP receptor intracellular domain. The non-TGFP receptor intracellular domain may be the intracellular domain or fragment thereof derived from TLR1, TLR2, TLR3, TLR4, TLR5, TLR6, TLR7, TLR8, TLR9, TLR10, CD28, 4-1BB (CD137), OX40 (CD134), CD3zeta, CD40, CD27, or a combination thereof.

ix. Activation-Conditional CAR

In some embodiments, the cargo or payload may be or may encode an activation-conditional chimeric antigen receptor, which is only expressed in an activated immune cell. The expression of the CAR may be coupled to activation conditional control region which refers to one or more nucleic acid sequences that induce the transcription and/or expression of a sequence e.g., a CAR under its control. Such activation conditional control regions may be promoters of genes that are upregulated during the activation of the immune effector cell e.g. IL2 promoter or NFAT binding sites.

x. CAR Targeting to Tumor Cells with Specific Proteoglycan Markers

In some embodiments, the cargo or payload may be or may encode a CAR that targets specific types of cancer cells. Human cancer cells and metastasis may express unique and otherwise abnormal proteoglycans, such as polysaccharide chains (e.g., chondroitin sulfate (CS), dermatan sulfate (DS or CSB), heparan sulfate (HS) and heparin). Accordingly, the CAR may be fused with a binding moiety that recognizes cancer associated proteoglycans. In one example, a CAR may be fused with VAR2CSA polypeptide (VAR2-CAR) that binds with high affinity to a specific type of chondroitin sulfate A (CSA) attached to proteoglycans. The extracellular ScFv portion of the CAR may be substituted with VAR2CSA variants comprising at least the minimal CSA binding domain, generating CARs specific to chondroitin sulfate A (CSA) modifications. Alternatively, the CAR may be fused with a split-protein binding system to generate a spy-CAR, in which the scFv portion of the CAR is substituted with one portion of a split-protein binding system such as SpyTag and Spy-catcher and the cancer-recognition molecules (e.g. scFv and or VAR2-CSA) are attached to the CAR through the split-protein binding system.

d. Nucleic Acids

The originator constructs and benchmark constructs of the present disclosure may comprise a payload region (which may also be referred to as a cargo region) which is a nucleic acid. The term “nucleic acid,” in its broadest sense, includes any compound and/or substance that comprise a polymer of nucleotides which may be referred to as polynucleotides. Exemplary nucleic acids or polynucleotides include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs) or hybrids thereof.

In some embodiments, the payload region comprises nucleic acid sequences encoding more than one cargo or payload.

In some embodiments, the payload region may be or encode a coding nucleic acid sequence.

In some embodiments, the payload region may be or encode a non-coding nucleic acid sequence.

In some embodiments, the payload region may be or encode both a coding and a non-coding nucleic acid sequence.

i. DNA

Deoxyribonucleic acid (DNA) is a molecule that carries genetic information for all living things and consists of two strands that wind around one another to form a shape known as a double helix. Each strand has a backbone made of alternating sugar (deoxyribose) and phosphate groups. Attached to each sugar is one of four bases: adenine (A), cytosine (C), guanine (G), and thymine (T). The two strands are held together by bonds between adenine and thymine or cytosine and guanine. The sequence of the bases along the backbones serves as instructions for assembling protein and RNA molecules.

In some embodiments, the payload region may be or encode a coding DNA.

In some embodiments, the payload region may be or encode a non-coding DNA.

In some embodiments, the payload region may be or encode both a coding and a non-coding DNA.

In some embodiments, the DNA may be modified. Suitable modifications include, but are not limited to, methylation, acetylation, phosphorylation, ubiquitination, and sumoylation. Vectors

In some embodiments, the originator constructs and/or benchmark constructs described herein can be or be encoded by vectors such as plasmids or viral vectors. In some embodiments, the originator constructs and/or benchmark constructs are or are encoded by viral vectors. Viral vectors may be, but are not limited to, Herpesvirus (HSV) vectors, retroviral vectors, adenoviral vectors, adeno-associated viral (AAV) vectors, lentiviral vectors, and the like. In some embodiments, the viral vectors are AAV vectors. In some embodiments, the viral vectors are lentiviral vectors. In some embodiments, the viral vectors are retroviral vectors. In some embodiments, the viral vectors are adenoviral vectors.

ii. Adeno-Associated Viral (AAVs) Vectors

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. Due to its relatively simple structure, easily manipulated using standard molecular biology techniques, this virus family is useful as a biological tool. The genome of the virus may be modified to contain a minimum of components for the assembly of a functional recombinant virus, or viral particle, which is loaded with or engineered to express or deliver a desired payload, which may be delivered to a target cell, tissue, organ, or organism.

The Parvoviridae family comprises the Dependovirus genus which includes adeno-associated viruses (AAV) capable of replication in vertebrate hosts including, but not limited to, human, primate, bovine, canine, equine, and ovine species.

The AAV vector genome is a linear, single-stranded DNA (ssDNA) molecule approximately 5,000 nucleotides (nts) in length. The AAV vector genome can comprise a payload region and at least one inverted terminal repeat (ITR) or ITR region. ITRs traditionally flank the coding nucleotide sequences for the non-structural proteins (encoded by Rep genes) and the structural proteins (encoded by capsid genes or Cap genes). While not wishing to be bound by theory, an AAV vector genome typically comprises two ITR sequences. The AAV vector genome comprises a characteristic T-shaped hairpin structure defined by the self-complementary terminal 145 nucleotides of the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In addition to the encoded heterologous payload, AAV vector genomes may comprise, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In some embodiments, the AAV vector genome comprises at least one control element which provides for the replication, transcription, and translation of a coding sequence encoded therein. Not all of the control elements need always be present as long as the coding sequence is capable of being replicated, transcribed, and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

AAV vector genomes of the present invention may be produced recombinantly and may be based on adeno-associated virus (AAV) parent or reference sequences. As used herein, a “vector genome” is any molecule or moiety which transports, transduces, or otherwise acts as a carrier of a heterologous molecule such as the nucleic acids described herein.

In addition to single stranded AAV vector genomes (e.g., ssAAVs), the present invention also provides for self-complementary AAV (scAAVs) vector genomes. scAAV vector genomes contain DNA strands which anneal together to form double stranded DNA. By skipping second strand synthesis, scAAVs allow for rapid expression in the cell.

In some embodiments, the AAV vector genome is an scAAV.

In some embodiments, the AAV vector genome is an ssAAV.

In some embodiments, the AAV vector genome may be part of an AAV particles where the serotype of the capsid may be, but is not limited to, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhErl.5, AAVhER1.14, AAVhErl.8, AAVhErl.16, AAVhErl.18, AAVhErl.35, AAVhErl.7, AAVhErl.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr—B7.3, AAV CBr—B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clvl-10, AAV CLv1-2, AAV CLv-12, AAV CLv1-3, AAV CLv-13, AAV CLv1-4, AAV Clvl-7, AAV Clvl-8, AAV Clvl-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, PHP.B, PUPA, G2B-26, G2B-13, TH1.1-32, and/or TH1.1-35 and variants thereof.Inverted Terminal Repeats (ITRs)

In some embodiments, the AAV vector genomes may comprise at least one ITR region and a payload region. In some embodiments, the vector genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into vector genomes of the invention may be comprised of naturally occurring polynucleotide sequences or recombinantly derived polynucleotide sequences.

The ITRs may be derived from the same serotype as the capsid or a derivative thereof. The ITR may be of a different serotype than the capsid. In some embodiments, the AAV particle has more than one ITR. In a non-limiting example, the AAV particle has a vector genome comprising two ITRs. In some embodiments, the ITRs are of the same serotype as one another. In another embodiment, the ITRs are of different serotypes. Non-limiting examples include zero, one or both of the ITRs having the same serotype as the capsid. In some embodiments both ITRs of the vector genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In some embodiments, the ITRs are 140-142 nucleotides in length. Non-limiting examples of ITR length are 102, 140, 141, 142, 145 nucleotides in length, and those having at least 95% identity thereto.

iii. Promoters

In some embodiments, the payload region of the vector genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

In some embodiments, the promoter is efficient when it drives expression of the polypeptide(s) encoded in the payload region of the vector genome of the AAV particle.

In some embodiments, the promoter is deemed to be efficient when it drives expression in the cell being targeted.

In some embodiments, the promoter drives expression of the payload for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, or 5-10 years.

In some embodiments, the promoter drives expression of the payload for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated.

Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US20110212529, the contents of which are herein incorporated by reference in their entirety)

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800, or 700-800.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In some embodiments, the promoter is a combination of a 382 nucleotide CMV-enhancer sequence and a 260 nucleotide CBA-promoter sequence.

In some embodiments, the vector genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

In some embodiments, the promoter is not cell specific.

In some embodiments, the vector genome comprises an engineered promoter.

In some embodiments, the vector genome comprises a promoter from a naturally expressed protein.

iv. Untranslated Regions (UTRs)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the vector genomes of the AAV particles of the invention to enhance expression in hepatic cell lines or liver.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In some embodiments, the 5′UTR in the vector genome includes a Kozak sequence.

In some embodiments, the 5′UTR in the vector genome does not include a Kozak sequence.

While not wishing to be bound by theory, wild-type 3′ UTRs are known to have stretches of adenosines and uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-a, possess two or more overlapping UUAUUUA(U/A)(U/A) nonamers. Class III AREs, such as, but not limited to, c-Jun and Myogenin, are less well defined. These U rich regions do not contain an AUUUA motif Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of vector genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the vector genome may include an oligo(dT) sequence for templated addition of a poly-A tail.

In some embodiments, the vector genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, e.g., a sequence in the region of positions 2-8 of the mature microRNA, which sequence has perfect Watson-Crick complementarity to the miRNA target sequence of the nucleic acid.

In some embodiments, the vector genome may be engineered to include, alter or remove at least one miRNA binding site, sequence, or seed region.

Any UTR from any gene known in the art may be incorporated into the vector genome of the AAV particle. These UTRs, or portions thereof, may be placed in the same orientation as in the gene from which they were selected or they may be altered in orientation or location. In some embodiments, the UTR used in the vector genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the vector genome of the AAV particle comprises at least one artificial UTRs which is not a variant of a wild-type UTR.

In some embodiments, the vector genome of the AAV particle comprises UTRs which have been selected from a family of transcripts whose proteins share a common function, structure, feature or property.

v. Polyadenylation Sequence

In some embodiments, the vector genome comprises at least one polyadenylation sequence between the 3′ end of the payload coding sequence and the 5′ end of the 3′ITR.

In some embodiments, the polyadenylation (poly-A) sequence may range from absent to about 500 nucleotides in length. The polyadenylation sequence may be, but is not limited to, 1, 2, 3, 4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310,311,312,313,314,315,316,317,318,319,320,321,322,323,324,325,326,327,328,329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and 500 nucleotides in length.

In some embodiments, the polyadenylation sequence is 50-100 nucleotides in length. In some embodiments, the polyadenylation sequence is 50-150 nucleotides in length. In some embodiments, the polyadenylation sequence is 50-160 nucleotides in length. In some embodiments, the polyadenylation sequence is 50-200 nucleotides in length. In some embodiments, the polyadenylation sequence is 60-100 nucleotides in length. In some embodiments, the polyadenylation sequence is 60-150 nucleotides in length. In some embodiments, the polyadenylation sequence is 60-160 nucleotides in length. In some embodiments, the polyadenylation sequence is 60-200 nucleotides in length. In some embodiments, the polyadenylation sequence is 70-100 nucleotides in length. In some embodiments, the polyadenylation sequence is 70-150 nucleotides in length. In some embodiments, the polyadenylation sequence is 70-160 nucleotides in length. In some embodiments, the polyadenylation sequence is 70-200 nucleotides in length. In some embodiments, the polyadenylation sequence is 80-100 nucleotides in length. In some embodiments, the polyadenylation sequence is 80-150 nucleotides in length. In some embodiments, the polyadenylation sequence is 80-160 nucleotides in length. In some embodiments, the polyadenylation sequence is 80-200 nucleotides in length. In some embodiments, the polyadenylation sequence is 90-100 nucleotides in length. In some embodiments, the polyadenylation sequence is 90-150 nucleotides in length. In some embodiments, the polyadenylation sequence is 90-160 nucleotides in length. In some embodiments, the polyadenylation sequence is 90-200 nucleotides in length.

vi. Linkers

Vector genomes may be engineered with one or more spacer or linker regions to separate coding or non-coding regions.

In some embodiments, the payload region of the vector genome may optionally encode one or more linker sequences. In some cases, the linker may be a peptide linker that may be used to connect the polypeptides encoded by the payload region (e.g., light and heavy antibody chains during expression). Some peptide linkers may be cleaved after expression to separate heavy and light chain domains, allowing assembly of mature antibodies or antibody fragments. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains from a single transcript. In some cases, two or more linkers are encoded by a payload region of the vector genome.

Internal ribosomal entry site (IRES) is a nucleotide sequence (>500 nucleotides) that allows for initiation of translation in the middle of an mRNA sequence (Kim, J. H. et al., 2011. PLoS One 6(4): e18556; the contents of which are herein incorporated by reference in its entirety). Use of an IRES sequence ensures co-expression of genes before and after the IRES, though the sequence following the IRES may be transcribed and translated at lower levels than the sequence preceding the IRES sequence.

2A peptides are small “self-cleaving” peptides (18-22 amino acids) derived from viruses such as foot-and-mouth disease virus (F2A), porcine teschovirus-1 (P2A), Thoseaasigna virus (T2A), or equine rhinitis A virus (E2A). The 2A designation refers specifically to a region of picornavirus polyproteins that lead to a ribosomal skip at the glycyl-prolyl bond in the C-terminus of the 2A peptide (Kim, J. H. et al., 2011. PLoS One 6(4): e18556; the contents of which are herein incorporated by reference in its entirety). This skip results in a cleavage between the 2A peptide and its immediate downstream peptide. As opposed to IRES linkers, 2A peptides generate stoichiometric expression of proteins flanking the 2A peptide and their shorter length can be advantageous in generating viral expression vectors.

Some payload regions encode linkers comprising furin cleavage sites. Furin is a calcium dependent serine endoprotease that cleaves proteins just downstream of a basic amino acid target sequence (Arg-X-(Arg/Lys)-Arg) (Thomas, G., 2002. Nature Reviews Molecular Cell Biology 3(10): 753-66; the contents of which are herein incorporated by reference in its entirety). Furin is enriched in the trans-golgi network where it is involved in processing cellular precursor proteins. Furin also plays a role in activating a number of pathogens. This activity can be taken advantage of for expression of polypeptides of the invention.

In some embodiments, the payload region may encode one or more linkers comprising cathepsin, matrix metalloproteinases or legumain cleavage sites. Such linkers are described e.g. by Cizeau and Macdonald in International Publication No. WO2008052322, the contents of which are herein incorporated in their entirety. Cathepsins are a family of proteases with unique mechanisms to cleave specific proteins. Cathepsin B is a cysteine protease and cathepsin D is an aspartyl protease. Matrix metalloproteinases are a family of calcium-dependent and zinc-containing endopeptidases. Legumain is an enzyme catalyzing the hydrolysis of (-Asn-Xaa-) bonds of proteins and small molecule substrates.

In some embodiments, payload regions may encode linkers that are not cleaved. Such linkers may include a simple amino acid sequence, such as a glycine rich sequence. In some cases, linkers may comprise flexible peptide linkers comprising glycine and serine residues. The linker may comprise flexible peptide linkers of different lengths, e.g. n×G4S, where n=1-10 (SEQ ID NO: 32) and the length of the encoded linker varies between 5 and 50 amino acids. In a non-limiting example, the linker may be 5×G4S (SEQ ID NO: 33). These flexible linkers are small and without side chains so they tend not to influence secondary protein structure while providing a flexible linker between antibody segments (George, R. A., et al., 2002. Protein Engineering 15(11): 871-9; Huston, J. S. et al., 1988. PNAS 85:5879-83; and Shan, D. et al., 1999. Journal of Immunology. 162(11):6589-95; the contents of each of which are herein incorporated by reference in their entirety). Furthermore, the polarity of the serine residues improves solubility and prevents aggregation problems.

In some embodiments, payload regions of the invention may encode small and unbranched serine-rich peptide linkers, such as those described by Huston et al. in U.S. Pat. No. 5,525,491, the contents of which are herein incorporated in their entirety. Polypeptides encoded by the payload region of the invention, linked by serine-rich linkers, have increased solubility.

In some embodiments, payload regions of the invention may encode artificial linkers, such as those described by Whitlow and Filpula in U.S. Pat. No. 5,856,456 and Ladner et al. in U.S. Pat. No. 4,946,778, the contents of each of which are herein incorporated by their entirety.

vii. Introns

In some embodiments, the payload region comprises at least one element to enhance the expression such as one or more introns or portions thereof. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In some embodiments, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

viii. Lentiviral Vectors

Lentiviral vectors are a type of retrovirus that can infect both dividing and nondividing cells because their viral shell can pass through the intact membrane of the nucleus of the target cell. Lentiviral vectors have the ability to deliver transgenes in tissues that had long appeared irremediably refractory to stable genetic manipulation. Lentivectors have also opened fresh perspectives for the genetic treatment of a wide array of hereditary as well as acquired disorders, and a real proposal for their clinical use seems imminent.

ix. RNA

Ribonucleic acid (RNA) is a molecule that is made up of nucleotides, which are ribose sugars attached to nitrogenous bases and phosphate groups. The nitrogenous bases include adenine (A), guanine (G), uracil (U), and cytosine (C). Generally, RNA mostly exists in the single-stranded form but can also exists double-stranded in certain circumstances. The length, form and structure of RNA is diverse depending on the purpose of the RNA. For example, the length of an RNA can vary from a short sequence (e.g., siRNA) to a long sequences (e.g., lncRNA), can be linear (e.g., mRNA) or circular (e.g., oRNA), and can either be a coding (e.g., mRNA) or a non-coding (e.g., lncRNA) sequence.

In some embodiments, the payload region may be or encode a coding RNA.

In some embodiments, the payload region may be or encode a non-coding RNA.

In some embodiments, the payload region may be or encode both a coding and a non-coding RNA.

In some embodiments, the payload region comprises nucleic acid sequences encoding more than one cargo or payload.

In some embodiments, the payload region comprises a nucleic acid sequence to enhance the expression of a gene. As a non-limiting example, the nucleic acid sequence is a messenger RNA (mRNA). As another non-limiting example, the nucleic acid sequence is a circular RNA (oRNA).

In some embodiments, the payload region comprises a nucleic acid sequence to reduce or inhibit the expression of a gene. As a non-limiting example, the nucleic acid sequence is a small interfering RNA (siRNA) or a microRNA (miRNA).

x. Small Interfering RNAs (siRNAs)

In some embodiments, the payload region may be or encode an RNA interference (RNAi) sequence which can be used to reduce or inhibit the expression of a gene. RNAi (also known as post-transcriptional gene silencing (PTGS), quelling, or co-suppression) is a post-transcriptional gene silencing process in which RNA molecules, in a sequence specific manner, reduce or inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2 nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, named microRNAs (miRNAs), elicit gene silencing by regulating the expression of mRNAs. The miRNAs-containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA-mediated down-regulation of gene expression may be caused by cleavage of the target mRNAs, translational inhibition of the target mRNAs, or mRNA decay. miRNA targeting sequences are usually located in the 3′-UTR of the target mRNAs. A single miRNA may target more than 100 transcripts from various genes, and one mRNA may be targeted by different miRNAs.

siRNA duplexes or dsRNA targeting a specific mRNA may be designed and synthesized in vitro and introduced into cells for activating RNAi processes. It has been previously shown that 21-nucleotide siRNA duplexes (termed small interfering RNAs) were capable of effecting potent and specific gene knockdown without inducing immune response in mammalian cells. Now post-transcriptional gene silencing by siRNAs has quickly emerged as a powerful tool for genetic analysis in mammalian cells and has the potential to produce novel therapeutics.

In vitro synthetized siRNA sequences may be introduced into cells in order to activate RNAi. An exogenous siRNA duplex, when it is introduced into cells, similar to the endogenous dsRNAs, can be assembled to form the RNA Induced Silencing Complex (RISC), a multiunit complex that interacts with RNA sequences that are complementary to one of the two strands of the siRNA duplex (e.g., the antisense strand). During the process, the sense strand (or passenger strand) of the siRNA is lost from the complex, while the antisense strand (or guide strand) of the siRNA is matched with its complementary RNA. In particular, the targets of siRNA containing RISC complexes are mRNAs presenting a perfect sequence complementarity. Then, siRNA mediated gene silencing occurs by cleaving, releasing and degrading the target.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases, it requires higher concentration of the ss-siRNA to achieve the effective gene silencing potency of the corresponding duplex.

xi. Design and Sequences of siRNA duplexes

Some guidelines for designing siRNAs have been proposed in the art. These guidelines generally recommend generating a 19-nucleotide duplexed region, symmetric 2-3 nucleotide 3′overhangs, 5′-phosphate and 3′-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U nucleotides in the 5′ terminal one-third of the antisense strand; and (iv) the absence of any GC stretch of more than 9 nucleotides in length. In accordance with such consideration, together with the specific sequence of a target gene, highly effective siRNA constructs essential for suppressing mammalian target gene expression may be readily designed.

In some embodiments, siRNA constructs (e.g., siRNA duplexes or encoded dsRNA) that target a specific gene are designed. Such siRNA constructs can specifically, suppress gene expression and protein production. In some aspects, the siRNA constructs are designed and used to selectively “knock out” gene variants in cells, e.g., mutated transcripts that are identified in patients or that are the cause of various diseases and/or disorders. In some aspects, the siRNA constructs are designed and used to selectively “knock down” variants of the gene in cells. In other aspects, the siRNA constructs are able to inhibit or suppress both the wild type and mutated versions of the gene.

In some embodiments, an siRNA sequence comprises a sense strand and a complementary antisense strand in which both strands are hybridized together to form a duplex structure. The antisense strand has sufficient complementarity to the mRNA sequence to direct target-specific RNAi, e.g., the siRNA sequence has a sequence sufficient to trigger the destruction of the target mRNA by the RNAi machinery or process.

In some embodiments, an siRNA sequence comprises a sense strand and a complementary antisense strand in which both strands are hybridized together to form a duplex structure and where the start site of the hybridization to the mRNA is between nucleotide 100 and 10,000 on the mRNA sequence. As a non-limiting example, the start site may be between nucleotide 100-150, 150-200, 200-250, 250-300, 300-350, 350-400, 400-450, 450-500, 500-550, 550-600, 600-650, 650-700, 700-70, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000, 4000-4050, 4050-4100, 4100-4150, 4150-4200, 4200-4250, 4250-4300, 4300-4350, 4350-4400, 4400-4450, 4450-4500, 4500-4550, 4550-4600, 4600-4650, 4650-4700, 4700-4750, 4750-4800, 4800-4850, 4850-4900, 4900-4950, 4950-5000, 5000-5050, 5050-5100, 5100-5150, 5150-5200, 5200-5250, 5250-5300, 5300-5350, 5350-5400, 5400-5450, 5450-5500, 5500-5550, 5550-5600, 5600-5650, 5650-5700, 5700-5750, 5750-5800, 5800-5850, 5850-5900, 5900-5950, 5950-6000, 6000-6050, 6050-6100, 6100-6150, 6150-6200, 6200-6250, 6250-6300, 6300-6350, 6350-6400, 6400-6450, 6450-6500, 6500-6550, 6550-6600, 6600-6650, 6650-6700, 6700-6750, 6750-6800, 6800-6850, 6850-6900, 6900-6950, 6950-7000, 7000-7050, 7050-7100, 7100-7150, 7150-7200, 7200-7250, 7250-7300, 7300-7350, 7350-7400, 7400-7450, 7450-7500, 7500-7550, 7550-7600, 7600-7650, 7650-7700, 7700-7750, 7750-7800, 7800-7850, 7850-7900, 7900-7950, 7950-8000, 8000-8050, 8050-8100, 8100-8150, 8150-8200, 8200-8250, 8250-8300, 8300-8350, 8350-8400, 8400-8450, 8450-8500, 8500-8550, 8550-8600, 8600-8650, 8650-8700, 8700-8750, 8750-8800, 8800-8850, 8850-8900, 8900-8950, 8950-9000, 9000-9050, 9050-9100, 9100-9150, 9150-9200, 9200-9250, 9250-9300, 9300-9350, 9350-9400, 9400-9450, 9450-9500, 9500-9550, 9550-9600, 9600-9650, 9650-9700, 9700-9750, 9750-9800, 9800-9850, 9850-9900, 9900-9950, 9950-10000 on the mRNA sequence.

In some embodiments, the antisense strand and target mRNA sequences have 100% complementarity. The antisense strand may be complementary to any part of the target mRNA sequence.

In other embodiments, the antisense strand and target mRNA sequences comprise at least one mismatch. As a non-limiting example, the antisense strand and the target mRNA sequence have at least 30%, 40%, 50%, 60%0, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementarity.

In some embodiments, the siRNA sequence has a length from about 10-50 or more nucleotides, e.g., each strand comprising 10-50 nucleotides (or nucleotide analogs). Preferably, the siRNA sequence has a length from about 15-30, e.g., 15, 16, 17, 18, 19, 20, 21, 22, 23, 24, 25, 26, 27, 28, 29, or 30 nucleotides in each strand, wherein one of the strands is sufficiently complementarity to a target region. In some embodiments, the siRNA sequence has a length from about 19 to 25, 19 to 24 or 19 to 21 nucleotides.

In some embodiments, the siRNA sequences can be synthetic RNA duplexes comprising about 19 nucleotides to about 25 nucleotides, and two overhanging nucleotides at the 3′-end. In some aspects, the siRNA constructs may be unmodified RNA molecules. In other aspects, the siRNA constructs may contain at least one modified nucleotide, such as base, sugar or backbone modifications.

In some embodiments, the siRNA sequences can be encoded in plasmid vectors, viral vectors or other nucleic acid expression vectors for delivery to a cell. DNA expression plasmids can be used to stably express the siRNA duplexes or dsRNA in cells and achieve long-term inhibition of the target gene expression. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA constructs.

In some embodiments, the sense and antisense strands of a siRNA duplex may be linked by a short spacer sequence, which may optionally be linked to additional flanking sequence, leading to the expression of a flanking arm-stem-loop structure termed primary microRNA (pri-miRNA). The pri-miRNA may be recognized and cleaved by Drosha and Dicer, and thus generate mature siRNA constructs.

In some embodiments, the siRNA duplexes or encoded dsRNA suppress (or degrade) target mRNA. Accordingly, the siRNA duplexes or encoded dsRNA can be used to substantially inhibit gene expression in a cell. In some aspects, the inhibition of gene expression refers to an inhibition by at least about 20%, preferably by at least about 30%, 40%, 50%, 60%, 70%, 80%, 85%, 90%, 95%, and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 40%, 50%, 60%, 70%, 80%, 85%, 90%, 95%, and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100%, or 95-100%.

In some embodiments, the siRNA constructs comprise a miRNA seed match for the target located in the guide strand. In another embodiment, the siRNA constructs comprise a miRNA seed match for the target located in the passenger strand. In yet another embodiment, the siRNA duplexes or encoded dsRNA targeting gene do not comprise a seed match for the target located in the guide or passenger strand.

In some embodiments, the siRNA duplexes or encoded dsRNA targeting the gene may have almost no significant full-length off targets for the guide strand. In another embodiment, the siRNA duplexes or encoded dsRNA targeting the gene may have almost no significant full-length off target effects for the passenger strand. The siRNA duplexes or encoded dsRNA targeting the gene may have less than 1%, 2%, 3%, 4%, 5%, 5%, 6%, 7%, 8%, 9%10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the guide or passenger strand.

In some embodiments, the siRNA duplexes or encoded dsRNA targeting the gene may have high activity in vitro. In another embodiment, the siRNA constructs may have low activity in vitro. In yet another embodiment, the siRNA duplexes or dsRNA targeting the gene may have high guide strand activity and low passenger strand activity in vitro.

In some embodiments, the siRNA constructs have a high guide strand activity and low passenger strand activity in vitro. The target knock-down (KD) by the guide strand may be at least 40%, 50%, 60%, 65%, 70%, 75%, 80%, 85%, 90%, 95%, 99%, 99.5% or 100%. The target knock-down by the guide strand may be 40-50%, 45-50%, 50-55%, 50-60%, 60-65%, 60-70%, 60-75%, 60-80%, 60-85%, 60-90%, 60-95%, 60-99%, 60-99.5%, 60-100%, 65-70%, 65-75%, 65-80%, 65-85%, 65-90%, 65-95%, 65-99%, 65-99.5%, 65-100%, 70-75%, 70-80%, 70-85%, 70-90%, 70-95%, 70-99%, 70-99.5%, 70-100%, 75-80%, 75-85%, 75-90%, 75-95%, 75-99%, 75-99.5%, 75-100%, 80-85%, 80-90%, 80-95%, 80-99%, 80-99.5%, 80-100%, 85-90%, 85-95%, 85-99%, 85-99.5%, 85-100%, 90-95%, 90-99%, 90-99.5%, 90-100%, 95-99%, 95-99.5%, 95-100%, 99-99.5%, 99-100% or 99.5-100%. As a non-limiting example, the target knock-down (KD) by the guide strand is greater than 70%. As a non-limiting example, the target knock-down (KD) by the guide strand is greater than 60%.

In some embodiments, the guide to passenger (G:P) (also referred to as the antisense to sense) strand ratio expressed is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The guide to passenger ratio refers to the ratio of the guide strands to the passenger strands after the intracellular processing of the pri-microRNA. For example, a 80:20 guide-to-passenger ratio would have 8 guide strands to every 2 passenger strands processed from the precursor. As a non-limiting example, the guide-to-passenger strand ratio is 8:2 in vitro. As a non-limiting example, the guide-to-passenger strand ratio is 8:2 in vivo. As a non-limiting example, the guide-to-passenger strand ratio is 9:1 in vitro. As a non-limiting example, the guide-to-passenger strand ratio is 9:1 in vivo.

In some embodiments, the guide to passenger (G:P) (also referred to as the antisense to sense) strand ratio expressed is greater than 1. In some embodiments, the guide to passenger (G:P) (also referred to as the antisense to sense) strand ratio expressed is greater than 2. In some embodiments, the guide to passenger (G:P) (also referred to as the antisense to sense) strand ratio expressed is greater than 5. In some embodiments, the guide to passenger (G:P) (also referred to as the antisense to sense) strand ratio expressed is greater than 10. In some embodiments, the guide to passenger (G:P) (also referred to as the antisense to sense) strand ratio expressed is greater than 20. In some embodiments, the guide to passenger (G:P) (also referred to as the antisense to sense) strand ratio expressed is greater than 50. In some embodiments, the guide to passenger (G:P) (also referred to as the antisense to sense) strand ratio expressed is at least 3:1. In some embodiments, the guide to passenger (G:P) (also referred to as the antisense to sense) strand ratio expressed is at least 5:1. In some embodiments, the guide to passenger (G:P) (also referred to as the antisense to sense) strand ratio expressed is at least 10:1. In some embodiments, the guide to passenger (G:P) (also referred to as the antisense to sense) strand ratio expressed is at least 20:1. In some embodiments, the guide to passenger (G:P) (also referred to as the antisense to sense) strand ratio expressed is at least 50:1.

In some embodiments, the passenger to guide (P:G) (also referred to as the sense to antisense) strand ratio expressed is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The passenger to guide ratio refers to the ratio of the passenger strands to the guide strands after the excision of the guide strand. For example, a 80:20 passenger to guide ratio would have 8 passenger strands to every 2 guide strands processed from the precursor. As a non-limiting example, the passenger-to-guide strand ratio is 80:20 in vitro. As a non-limiting example, the passenger-to-guide strand ratio is 80:20 in vivo. As a non-limiting example, the passenger-to-guide strand ratio is 8:2 in vitro. As a non-limiting example, the passenger-to-guide strand ratio is 8:2 in vivo. As a non-limiting example, the passenger-to-guide strand ratio is 9:1 in vitro. As a non-limiting example, the passenger-to-guide strand ratio is 9:1 in vivo.

In some embodiments, the passenger to guide (P:G) (also referred to as the sense to antisense) strand ratio expressed is greater than 1. In some embodiments, the passenger to guide (P:G) (also referred to as the sense to antisense) strand ratio expressed is greater than 2. In some embodiments, the passenger to guide (P:G) (also referred to as the sense to antisense) strand ratio expressed is greater than 5. In some embodiments, the passenger to guide (P:G) (also referred to as the sense to antisense) strand ratio expressed is greater than 10. In some embodiments, the passenger to guide (P:G) (also referred to as the sense to antisense) strand ratio expressed is greater than 20. In some embodiments, the passenger to guide (P:G) (also referred to as the sense to antisense) strand ratio expressed is greater than 50. In some embodiments, the passenger to guide (P:G) (also referred to as the sense to antisense) strand ratio expressed is at least 3:1. In some embodiments, the passenger to guide (P:G) (also referred to as the sense to antisense) strand ratio expressed is at least 5:1. In some embodiments, the passenger to guide (P:G) (also referred to as the sense to antisense) strand ratio expressed is at least 10:1. In some embodiments, the passenger to guide (P:G) (also referred to as the sense to antisense) strand ratio expressed is at least 20:1. In some embodiments, the passenger to guide (P:G) (also referred to as the sense to antisense) strand ratio expressed is at least 50:1.

In some embodiments, a passenger-guide strand duplex is considered effective when the pri- or pre-microRNAs demonstrate, by methods known in the art and described herein, greater than 2-fold guide to passenger strand ratio when processing is measured. As a non-limiting examples, the pri- or pre-microRNAs demonstrate great than 2-fold, 3-fold, 4-fold, 5-fold, 6-fold, 7-fold, 8-fold, 9-fold, 10-fold, 11-fold, 12-fold, 13-fold, 14-fold, 15-fold, or 2 to 5-fold, 2 to 10-fold, 2 to 15-fold, 3 to 5-fold, 3 to 10-fold, 3 to 15-fold, 4 to 5-fold, 4 to 10-fold, 4 to 15-fold, 5 to 10-fold, 5 to 15-fold, 6 to 10-fold, 6 to 15-fold, 7 to 10-fold, 7 to 15-fold, 8 to 10-fold, 8 to 15-fold, 9 to 10-fold, 9 to 15-fold, 10 to 15-fold, 11 to 15-fold, 12 to 15-fold, 13 to 15-fold, or 14 to 15-fold guide to passenger strand ratio when processing is measured.

In some embodiments, the vector genome encoding the dsRNA comprises a sequence which is at least 60%, 65%, 70%, 75%, 80%, 85%, 90%, 95%, 99% or more than 99% of the full length of the construct. As a non-limiting example, the vector genome comprises a sequence which is at least 80% of the full length sequence of the construct.

In some embodiments, the siRNA constructs may be used to silence a wild type or mutant gene by targeting at least one exon on the sequence.

siRNA Modification

In some embodiments, the siRNA constructs, when not delivered as a precursor or DNA, may be chemically modified to modulate some features of RNA molecules, such as, but not limited to, increasing the stability of siRNAs in vivo. The chemically modified siRNA constructs can be used in human therapeutic applications and can be improved without compromising the RNAi activity of the siRNA constructs. As a non-limiting example, the siRNA constructs modified at both the 3′ and the 5′ end of both the sense strand and the antisense strand.

In some embodiments, the modified nucleotides may be on just the sense strand.

In some embodiments, the modified nucleotides may be on just the antisense strand.

In some embodiments, the modified nucleotides may be in both the sense and antisense strands.

In some embodiments, the chemically modified nucleotide does not affect the ability of the antisense strand to pair with the target mRNA sequence. microRNA scaffolds.

In some embodiments, the siRNA constructs may be encoded in a polynucleotide sequence which also comprises a microRNA (miRNA) scaffold construct. As used herein a “microRNA (miRNA) scaffold construct” is a framework or starting molecule that forms the sequence or structural basis against which to design or make a subsequent molecule.

In some embodiments, the miRNA scaffold construct comprises at least one 5′ flanking region. As a non-limiting example, the 5′ flanking region may comprise a 5′ flanking sequence which may be of any length and may be derived in whole or in part from wild type microRNA sequence or be a completely artificial sequence.

In some embodiments, the miRNA scaffold construct comprises at least one 3′ flanking region. As a non-limiting example, the 3′ flanking region may comprise a 3′ flanking sequence which may be of any length and may be derived in whole or in part from wild type microRNA sequence or be a completely artificial sequence.

In some embodiments, the miRNA scaffold construct comprises at least one loop motif region. As a non-limiting example, the loop motif region may comprise a sequence which may be of any length.

In some embodiments, the miRNA scaffold construct comprises a 5′ flanking region, a loop motif region and/or a 3′ flanking region.

In some embodiment, at least one payload (e.g., siRNA, miRNA or other RNAi agent described herein) may be encoded by a polynucleotide which may also comprise at least one miRNA scaffold construct. The miRNA scaffold construct may comprise a 5′ flanking sequence which may be of any length and may be derived in whole or in part from wild type microRNA sequence or be completely artificial. The 3′ flanking sequence may mirror the 5′ flanking sequence and/or a 3′ flanking sequence in size and origin. Either flanking sequence may be absent. The 3′ flanking sequence may optionally contain one or more CNNC motifs, where “N” represents any nucleotide.

In some embodiments, the 5′ arm of the stem loop structure of the polynucleotide comprising or encoding the miRNA scaffold construct comprises a sequence encoding a sense sequence.

In some embodiments, the 3′ arm of the stem loop of the polynucleotide comprising or encoding the miRNA scaffold construct comprises a sequence encoding an antisense sequence. The antisense sequence, in some instances, comprises a “G” nucleotide at the 5′ most end.

In some embodiments, the sense sequence may reside on the 3′ arm while the antisense sequence resides on the 5′ arm of the stem of the stem loop structure of the polynucleotide comprising or encoding the miRNA scaffold construct.

In some embodiments, the sense and antisense sequences may be completely complementary across a substantial portion of their length. In other embodiments the sense sequence and antisense sequence may be at least 70, 80, 90, 95 or 99% complementarity across independently at least 50, 60, 70, 80, 85, 90, 95, or 99% of the length of the strands.

Neither the identity of the sense sequence nor the homology of the antisense sequence need to be 100% complementarity to the target sequence.

In some embodiments, separating the sense and antisense sequence of the stem loop structure of the polynucleotide is a loop sequence (also known as a loop motif, linker or linker motif). The loop sequence may be of any length, between 4-30 nucleotides, between 4-20 nucleotides, between 4-15 nucleotides, between 5-15 nucleotides, between 6-12 nucleotides, 6 nucleotides, 7 nucleotides, 8 nucleotides, 9, nucleotides, 10, nucleotides, 11 nucleotides, 12 nucleotides, 13 nucleotides, 14 nucleotides, and/or 15 nucleotides.

In some embodiments, the loop sequence comprises a nucleic acid sequence encoding at least one UGUG motif. In some embodiments, the nucleic acid sequence encoding the UGUG motif is located at the 5′ terminus of the loop sequence.

In some embodiments, spacer regions may be present in the polynucleotide to separate one or more modules (e.g., 5′ flanking region, loop motif region, 3′ flanking region, sense sequence, antisense sequence) from one another. There may be one or more such spacer regions present.

In some embodiments, a spacer region of between 8-20, e.g., 8, 9, 10, 11, 12, 13, 14, 15, 16, 17, 18, 19, or 20 nucleotides may be present between the sense sequence and a flanking region sequence.

In some embodiments, the length of the spacer region is 13 nucleotides and is located between the 5′ terminus of the sense sequence and the 3′ terminus of the flanking sequence. In some embodiments, a spacer is of sufficient length to form approximately one helical turn of the sequence.

In some embodiments, a spacer region of between 8-20, e.g., 8, 9, 10, 11, 12, 13, 14, 15, 16, 17, 18, 19, or 20 nucleotides may be present between the antisense sequence and a flanking sequence.

In some embodiments, the spacer sequence is between 10-13, e.g., 10, 11, 12 or 13 nucleotides and is located between the 3′ terminus of the antisense sequence and the 5′ terminus of a flanking sequence. In some embodiments, a spacer is of sufficient length to form approximately one helical turn of the sequence.

In some embodiments, the polynucleotide comprises in the 5′ to 3′ direction, a 5′ flanking sequence, a 5′ arm, a loop motif, a 3′ arm and a 3′ flanking sequence. As a non-limiting example, the 5′ arm may comprise a sense sequence and the 3′ arm comprises the antisense sequence. In another non-limiting example, the 5′ arm comprises the antisense sequence and the 3′ arm comprises the sense sequence.

In some embodiments, the 5′ arm, payload (e.g., sense and/or antisense sequence), loop motif and/or 3′ arm sequence may be altered (e.g., substituting 1 or more nucleotides, adding nucleotides and/or deleting nucleotides). The alteration may cause a beneficial change in the function of the construct (e.g., increase knock-down of the target sequence, reduce degradation of the construct, reduce off target effect, increase efficiency of the payload, and reduce degradation of the payload).

In some embodiments, the miRNA scaffold construct of the polynucleotides is aligned in order to have the rate of excision of the guide strand be greater than the rate of excision of the passenger strand. The rate of excision of the guide or passenger strand may be, independently, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99%. As a non-limiting example, the rate of excision of the guide strand is at least 80%. As another non-limiting example, the rate of excision of the guide strand is at least 90%. In some embodiments, the rate of excision of the guide strand is greater than the rate of excision of the passenger strand. In one aspect, the rate of excision of the guide strand may be at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99% greater than the passenger strand.

In some embodiments, the efficiency of excision of the guide strand is at least 60%, 65%, 70%, 75%, 80%, 85%, 90%, 95%, 99% or more than 99%. As a non-limiting example, the efficiency of the excision of the guide strand is greater than 80%.

In some embodiments, the efficiency of the excision of the guide strand is greater than the excision of the passenger strand from the miRNA scaffold construct. The excision of the guide strand may be 2, 3, 4, 5, 6, 7, 8, 9, 10 or more than 10 times more efficient than the excision of the passenger strand from the miRNA scaffold construct.

In some embodiments, the miRNA scaffold construct comprises a dual-function targeting polynucleotide. As used herein, a “dual-function targeting” polynucleotide is a polynucleotide where both the guide and passenger strands knock down the same target or the guide and passenger strands knock down different targets.

In some embodiments, the miRNA scaffold construct of the polynucleotides described herein may comprise a 5′ flanking region, a loop motif region and a 3′ flanking region.

In some embodiments, the polynucleotide is designed using at least one of the following properties: loop variant, seed mismatch/bulge/wobble variant, stem mismatch, loop variant and vassal stem mismatch variant, seed mismatch and basal stem mismatch variant, stem mismatch and basal stem mismatch variant, seed wobble and basal stem wobble variant, or a stem sequence variant.

In some embodiments, the miRNA scaffold construct may be a natural pri-miRNA scaffold.

In some embodiments, the selection of a miRNA scaffold construct is determined by a method of comparing polynucleotides in pri-miRNA.

In some embodiments, the selection of a miRNA scaffold construct is determined by a method of comparing polynucleotides in natural pri-miRNA and synthetic pri-miRNA.

xii. Transfer RNA (tRNA)

Transfer RNAs (tRNAs) are RNA molecules that translate mRNA into proteins. tRNA include a cloverleaf structure that comprise a 3′ acceptor site, 5′ terminal phosphate, D arm, T arm, and anticodon arm. The main purpose of a tRNA is to carry amino acids on its 3′ acceptor site to a ribosome complex with the help of aminoacyl-tRNA synthetases which are enzymes that load the appropriate amino acid onto a free tRNA to synthesize proteins. Once an amino acid is bound to tRNA, the tRNA is considered an aminoacyl-tRNA. The type of amino acid on a tRNA is dependent on the mRNA codon. The anticodon arm of the tRNA is the site of the anticodon, which is complementary to an mRNA codon and dictates which amino acid to carry. tRNAs are also known to have a role in the regulation of apoptosis by acting as a cytochrome c scavenger.

In some embodiments, the originator construct and/or the benchmark construct comprises or encodes a tRNA.

xiii. microRNA (miRNA)

microRNAs (or miRNA) are 19-25 nucleotide long noncoding RNAs that bind to the 3′UTR of nucleic acid molecules and down-regulate gene expression either by reducing nucleic acid molecule stability or by inhibiting translation. The originator constructs and/or benchmark constructs may comprise one or more microRNA target sequences, microRNA sequences, or microRNA seeds.

A microRNA sequence comprises a “seed” region, e.g., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. The bases of the microRNA seed have complete complementarity with the target sequence. By engineering microRNA target sequences into the 3′ UTR of the mRNA one can target the molecule for degradation or reduced translation, provided the microRNA in question is available. This process will reduce the hazard of off target effects upon nucleic acid molecule delivery.

As used herein, the term “microRNA site” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Non-limiting examples of tissues where microRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-ld, miR-149), kidney (miR-192, miR-194, miR-204), and lung epithelial cells (let-7, miR-133, miR-126). MicroRNA can also regulate complex biological processes such as angiogenesis (miR-132).

For example, if the nucleic acid molecule is an mRNA and is not intended to be delivered to the liver but ends up there, then miR-122, a microRNA abundant in liver, can inhibit the expression of the gene of interest if one or multiple target sites of miR-122 are engineered into the 3′ UTR of the mRNA. Introduction of one or multiple binding sites for different microRNA can be engineered to further decrease the longevity, stability, and protein translation of a mRNA.

Conversely, microRNA binding sites can be engineered out of (e.g., removed from) sequences in which they naturally occur in order to increase protein expression in specific tissues. For example, miR-122 binding sites may be removed to improve protein expression in the liver. Regulation of expression in multiple tissues can be accomplished through introduction or removal or one or several microRNA binding sites.

xiv. Messenger RNA (mRNA)

In some embodiments, the originator constructs and/or benchmark constructs may be mRNA. As used herein, the term “messenger RNA” (mRNA) refers to any polynucleotide which encodes a target of interest and which is capable of being translated to produce the encoded target of interest in vitro, in vivo, in situ or ex vivo.

Generally, an mRNA molecule comprises at least a coding region, a 5′ untranslated region (UTR), a 3′ UTR, a 5′ cap and a poly-A tail. In some aspects, one or more structural and/or chemical modifications or alterations may be included in the RNA which can reduce the innate immune response of a cell in which the mRNA is introduced. As used herein, a “structural” feature or modification is one in which two or more linked nucleotides are inserted, deleted, duplicated, inverted or randomized in a nucleic acid without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”.

Generally, the shortest length of a region of the originator constructs and/or benchmark constructs can be the length of a nucleic acid sequence that is sufficient to encode for a dipeptide, a tripeptide, a tetrapeptide, a pentapeptide, a hexapeptide, a heptapeptide, an octapeptide, a nonapeptide, or a decapeptide. In another embodiment, the length may be sufficient to encode a peptide of 2-30 amino acids, e.g. 5-30, 10-30, 2-25, 5-25, 10-25, or 10-20 amino acids. The length may be sufficient to encode for a peptide of at least 11, 12, 13, 14, 15, 17, 20, 25 or 30 amino acids, or a peptide that is no longer than 40 amino acids, e.g. no longer than 35, 30, 25, 20, 17, 15, 14, 13, 12, 11 or 10 amino acids.

Generally, the length of the region of the mRNA encoding a target of interest is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the mRNA includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

In some embodiments, the region or regions flanking the region encoding the target of interest may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

In some embodiments, the mRNA comprises a tailing sequence which can range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

In some embodiments, the mRNA comprises a capping sequence which comprises a single cap or a series of nucleotides forming the cap. The capping sequence may be from 1 to 10, e.g. 2-9, 3-8, 4-7, 1-5, 5-10, or at least 2, or 10 or fewer nucleotides in length. In some embodiments, the caping sequence is absent.

In some embodiments, the mRNA comprises a region comprising a start codon. The region comprising the start codon may range from 3 to 40, e.g., 5-30, 10-20, 15, or at least 4, or 30 or fewer nucleotides in length.

In some embodiments, the mRNA comprises a region comprising a stop codon. The region comprising the stop codon may range from 3 to 40, e.g., 5-30, 10-20, 15, or at least 4, or 30 or fewer nucleotides in length.

In some embodiments, the mRNA comprises a region comprising a restriction sequence. The region comprising the restriction sequence may range from 3 to 40, e.g., 5-30, 10-20, 15, or at least 4, or 30 or fewer nucleotides in length. xv. Untranslated Regions (UTRs)

In some embodiments, the mRNA comprises at least one untranslated region (UTR) which flanks the region encoding the target of interest. UTRs are transcribed but not translated.

The 5′ UTR starts at the transcription start site and continues to the start codon but does not include the start codon; whereas, the 3′ UTR starts immediately following the stop codon and continues until the transcriptional termination signal. While not wishing to be bound by theory, the UTRs may have a regulatory role in terms of translation and stability of the nucleic acid.

Natural 5′ UTRs usually include features which have a role in translation initiation as they tend to include Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTR also have been known to form secondary structures which are involved in elongation factor binding.

3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo. Introduction, removal or modification of 3′ UTR AU rich elements (AREs) can be used to modulate the stability of mRNA. For example, one or more copies of an ARE can be introduced to make mRNA less stable and thereby curtail translation and decrease production of the resultant protein. Alternatively, AREs can be identified and removed or mutated to increase the intracellular stability and thus increase translation and production of the resultant protein.

In some embodiments, the introduction of features often expressed in genes of target organs the stability and protein production of the mRNA can be enhanced in a specific organ and/or tissue. As a non-limiting example, the feature can be a UTR. As another example, the feature can be introns or portions of introns sequences. xvi. 5′ Capping

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap nucleotide and the 5′-terminal transcribed sense nucleotide of the mRNA molecule. This 5′-guanylate cap may then be methylated to generate an N7-methyl-guanylate nucleotide. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to mRNA may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, MA) may be used with 6-thio-guanosine nucleotides according to the manufacturer's instructions to create a phosphorothioate linkage in the 5′-ppp-5′ cap.

Additional modified guanosine nucleotides may be used such as a-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (e.g., endogenous, wild-type or physiological) 5′-caps in their chemical structure, while retaining cap function. Cap analogs may be chemically (e.g., non-enzymatically) or enzymatically synthesized and/or linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (e.g., N7,3′-O-dimethyl-guanosine-5′-triphosphate-5′-guanosine (m⁷G-3′mppp-G; which may equivalently be designated 3′-O-Me-m7G(5′)ppp(5′)G). The 3′-O atom of the other, unmodified, guanine becomes linked to the 5′-terminal nucleotide of the capped nucleic acid molecule (e.g. an mRNA). The N7- and 3′-O-methlyated guanine provides the terminal moiety of the capped nucleic acid molecule (e.g. mRNA).

Another exemplary cap is mCAP, which is similar to ARCA but has a 2′-O-methyl group on guanosine (e.g., N7,2′-O-dimethyl-guanosine-5′-triphosphate-5′-guanosine, m⁷Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

mRNA may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′ cap structures are those which, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5*)ppp(5*)N,pN2p (cap 0), 7mG(5*)ppp(5*)NlmpNp (cap 1), and 7mG(5*)-ppp(5′)NlmpN2mp (cap 2).

In some embodiments, the 5′ terminal caps may include endogenous caps or cap analogs.

In some embodiments, a 5′ terminal cap may comprise a guanosine analog. Useful guanosine analogs include, but are not limited to, inosine, N1-methyl-guanosine, 2′-fluoro-guanosine, 7-deaza-guanosine, 8-oxo-guanosine, 2-amino-guanosine, LNA-guanosine, and 2-azido-guanosine.

xvii. IRES Sequences

In some embodiments, the mRNA may contain an internal ribosome entry site (IRES). First identified as a feature Picornavirus RNA, IRES plays an important role in initiating protein synthesis in absence of the 5′ cap structure. An IRES may act as the sole ribosome binding site, or may serve as one of multiple ribosome binding sites of an mRNA. An mRNA that contains more than one functional ribosome binding site may encode several peptides or polypeptides that are translated independently by the ribosomes. Non-limiting examples of IRES sequences that can be used include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

xviii. Poly A Tails

During RNA processing, a long chain of adenine nucleotides (poly-A tail) may be added to a polynucleotide such as an mRNA molecules in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail of a certain length.

In some embodiments, the length of a poly-A tail is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the mRNA includes a poly-A tail from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall mRNA. This design may be based on the length of the region coding for a target of interest, the length of a particular feature or region (such as a flanking region), or based on the length of the ultimate product expressed from the mRNA.

In this context the poly-A tail may be 10, 20, 30, 40, 50, 60, 70, 80, 90, or 100% greater in length than the mRNA or feature thereof. The poly-A tail may also be designed as a fraction of mRNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of mRNA for poly-A binding protein may enhance expression.

Additionally, multiple distinct mRNA may be linked together to the PABP (Poly-A binding protein) through the 3′-end using modified nucleotides at the 3′-terminus of the poly-A tail. Transfection experiments can be conducted in relevant cell lines and protein production can be assayed by ELISA at 12 hr, 24 hr, 48 hr, 72 hr and day 7 post-transfection.

In some embodiments, the mRNA are designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly-A tail.

xix. Stop Codons

In some embodiments, the mRNA may include one stop codon. In some embodiments, the mRNA may include two stop codons. In some embodiments, the mRNA may include three stop codons. In some embodiments, the mRNA may include at least one stop codon. In some embodiments, the mRNA may include at least two stop codons. In some embodiments, the mRNA may include at least three stop codons. As non-limiting examples, the stop codon may be selected from UGA, YAA and UAG.

In some embodiments, the mRNA includes the stop codon TGA and one additional stop codon. In a further embodiment the addition stop codon may be TAA.

xx. Circular RNA (oRNA)

In some embodiments, the originator construct and/or the benchmark construct is a circular RNA (oRNA). As used herein, the terms “oRNA” or “circular RNA” are used interchangeably and can refer to a RNA that forms a circular structure through covalent or non-covalent bonds.

In some embodiments, the oRNA may be non-immunogenic in a mammal (e.g., a human, non-human primate, rabbit, rat, and mouse).

In some embodiments, the oRNA may be capable of replicating or replicates in a cell from an aquaculture animal (e.g., fish, crabs, shrimp, oysters etc.), a mammalian cell, a cell from a pet or zoo animal (e.g., cats, dogs, lizards, birds, lions, tigers and bears etc.), a cell from a farm or working animal (e.g., horses, cows, pigs, chickens etc.), a human cell, cultured cells, primary cells or cell lines, stem cells, progenitor cells, differentiated cells, germ cells, cancer cells (e.g., tumorigenic, metastatic), non-tumorigenic cells (e.g., normal cells), fetal cells, embryonic cells, adult cells, mitotic cells, non-mitotic cells, or any combination thereof.

In some embodiments, the oRNA has a half-life of at least that of a linear counterpart. In some embodiments, the oRNA has a half-life that is increased over that of a linear counterpart. In some embodiments, the half-life is increased by about 5%, 10%, 15%, 20%, 25%, 30%, 35%, 40%, 45%, 50%, or greater. In some embodiments, the oRNA has a half-life or persistence in a cell for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In some embodiments, the oRNA has a half-life or persistence in a cell for no more than about 10 mins to about 7 days, or no more than about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 24 hours (1 day), 36 hours (1.5 days), 48 hours (2 days), 60 hours (2.5 days), 72 hours (3 days), 4 days, 5 days, 6 days, or 7 days.

In some embodiments, the oRNA has a half-life or persistence in a cell while the cell is dividing. In some embodiments, the oRNA has a half-life or persistence in a cell post division. In certain embodiments, the oRNA has a half-life or persistence in a dividing cell for greater than about 10 minutes to about 30 days, or at least about 10 minutes, 15 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, the oRNA modulates a cellular function, e.g., transiently or long term. In certain embodiments, the cellular function is stably altered, such as a modulation that persists for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer. In certain embodiments, the cellular function is transiently altered, e.g., such as a modulation that persists for no more than about 30 mins to about 7 days, or no more than about 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours (1 day), 36 hours (1.5 days), 48 hours (2 days), 60 hours (2.5 days), 72 hours (3 days), 4 days, 5 days, 6 days, or 7 days.

In some embodiments, the oRNA is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the oRNA may be of a sufficient size to accommodate a binding site for a ribosome.

In some embodiments, the maximum size of the oRNA may be limited by the ability of packaging and delivering the RNA to a target. In some embodiments, the size of the oRNA is a length sufficient to encode polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides may be useful.

In some embodiments, the oRNA comprises one or more elements described elsewhere herein. In some embodiments, the elements may be separated from one another by a spacer sequence or linker. In some embodiments, the elements may be separated from one another by 1 nucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides.

In some embodiments, one or more elements are contiguous with one another, e.g., lacking a spacer element.

In some embodiments, one or more elements is conformationally flexible. In some embodiments, the conformational flexibility is due to the sequence being substantially free of a secondary structure.

In some embodiments, the oRNA comprises a secondary or tertiary structure that accommodates a binding site for a ribosome, translation, or rolling circle translation.

In some embodiments, the oRNA comprises particular sequence characteristics. For example, the oRNA may comprise a particular nucleotide composition. In some such embodiments, the oRNA may include one or more purine rich regions (adenine or guanosine). In some such embodiments, the oRNA may include one or more purine rich regions (adenine or guanosine). In some embodiments, the oRNA may include one or more AU rich regions or elements (AREs). In some embodiments, the oRNA may include one or more adenine rich regions.

In some embodiments, the oRNA comprises one or more modifications described elsewhere herein.

In some embodiments, the oRNA comprises one or more expression sequences and is configured for persistent expression in a cell of a subject in vivo. In some embodiments, the oRNA is configured such that expression of the one or more expression sequences in the cell at a later time point is equal to or higher than an earlier time point. In such embodiments, the expression of the one or more expression sequences can be either maintained at a relatively stable level or can increase over time. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

xxi. Regulatory Elements

In some embodiments, the oRNA comprises a regulatory element. As used herein, a “regulatory element” is a sequence that modifies expression of an expression sequence. The regulatory element may include a sequence that is located adjacent to a payload or cargo region. The regulatory element may be operatively linked operatively to a payload or cargo region.

In some embodiments, a regulatory element may increase an amount of payload or cargo expressed as compared to an amount expressed when no regulatory element exists. As a non-limiting example, one regulatory element can increase an amount of payloads or cargos expressed for multiple payload or cargo sequences attached in tandem.

In some embodiments, a regulatory element may comprise a sequence to selectively initiates or activates translation of a payload or cargo.

In some embodiments, a regulatory element may comprise a sequence to initiate degradation of the oRNA or the payload or cargo. Non-limiting examples of the sequence to initiate degradation include, but is not limited to, riboswitch aptazymes and miRNA binding sites.

In some embodiments, a regulatory element can modulate translation of the payload or cargo in the oRNA. The modulation can create an increase (enhancer) or decrease (suppressor) in the payload or cargo. The regulatory element may be located adjacent to the payload or cargo (e.g., on one side or both sides of the payload or cargo).

In some embodiments, a translation initiation sequence functions as a regulatory element. In some embodiments, the translation initiation sequence comprises an AUG/ATG codon. In some embodiments, a translation initiation sequence comprises any eukaryotic start codon such as, but not limited to, AUG/ATG, CUG/CTG, GUG/GTG, UUG/TTG, ACG, AUC/ATC, AUU, AAG, AUA/ATA, or AGG. In some embodiments, a translation initiation sequence comprises a Kozak sequence. In some embodiments, translation begins at an alternative translation initiation sequence, e.g., translation initiation sequence other than AUG/ATG codon, under selective conditions, e.g., stress induced conditions. As a non-limiting example, the translation of the circular polyribonucleotide may begin at alternative translation initiation sequence, such as ACG. As another non-limiting example, the circular polyribonucleotide translation may begin at alternative translation initiation sequence, CUG/CTG. As another non-limiting example, the translation may begin at alternative translation initiation sequence, GUG/GTG. As yet another non-limiting example, the translation may begin at a repeat-associated non-AUG (RAN) sequence, such as an alternative translation initiation sequence that includes short stretches of repetitive RNA e.g. CGG, GGGGCC, CAG, CTG.

xxii. Masking Agents

Masking any of the nucleotides flanking a codon that initiates translation may be used to alter the position of translation initiation, translation efficiency, length and/or structure of the oRNA. In some embodiments, a masking agent may be used near the start codon or alternative start codon in order to mask or hide the codon to reduce the probability of translation initiation at the masked start codon or alternative start codon. Non-limiting examples of masking agents include antisense locked nucleic acids (LNA) oligonucleotides and exon junction complexes (EJCs). In some embodiments, a masking agent may be used to mask a start codon of the oRNA in order to increase the likelihood that translation will initiate at an alternative start codon.

xxiii. Translation Initiation Sequence

In some embodiments, the oRNA encodes a polypeptide or peptide and may comprise a translation initiation sequence. The translation initiation sequence may comprise, but is not limited to a start codon, a non-coding start codon, a Kozak sequence or a Shine-Dalgarno sequence. The translation initiation sequence may be located adjacent to the payload or cargo (e.g., on one side or both sides of the payload or cargo).

In some embodiments, the translation initiation sequence provides conformational flexibility to the oRNA. In some embodiments, the translation initiation sequence is within a substantially single stranded region of the oRNA.

The oRNA may include more than 1 start codon such as, but not limited to, at least 2, at least 3, at least 4, at least 5, at least 6, at least 7, at least 8, at least 9, at least 10, at least 11, at least 12, at least 13, at least 14, at least 15 or more than 15 start codons. Translation may initiate on the first start codon or may initiate downstream of the first start codon.

In some embodiments, the oRNA may initiate at a codon which is not the first start codon, e.g., AUG. Translation of the circular polyribonucleotide may initiate at an alternative translation initiation sequence, such as, but not limited to, ACG, AGG, AAG, CUG/CTG, GUG/GTG, AUA/ATA, AUU/ATT, UUG/TTG. In some embodiments, translation begins at an alternative translation initiation sequence under selective conditions, e.g., stress induced conditions. As a non-limiting example, the translation of the oRNA may begin at alternative translation initiation sequence, such as ACG. As another non-limiting example, the oRNA translation may begin at alternative translation initiation sequence, CUG/CTG. As yet another non-limiting example, the oRNA translation may begin at alternative translation initiation sequence, GTG/GUG. As yet another non-limiting example, the oRNA may begin translation at a repeat-associated non-AUG (RAN) sequence, such as an alternative translation initiation sequence that includes short stretches of repetitive RNA e.g. CGG, GGGGCC, CAG, CTG.

xxiv. IRES Sequences

In some embodiments, the oRNA described herein comprises an internal ribosome entry site (IRES) element capable of engaging an eukaryotic ribosome. In some embodiments, the IRES element is at least about 5 nucleotides, at least about 8 nucleotides, at least about 9 nucleotides, at least about 10 nucleotides, at least about 15 nucleotides, at least about 20 nucleotides, at least about 25 nucleotides, at least about 30 nucleotides, at least about 40 nucleotides, at least about 50 nucleotides, at least about 100 nucleotides, at least about 200 nucleotides, at least about 250 nucleotides, at least about 350 nucleotides, or at least about 500 nucleotides. In one embodiment, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES element is at least partially derived from a virus, for instance, it can be derived from a viral IRES element, such as ABPV_IGRpred, AEV, ALPV_IGRpred, BQCV_IGRpred, BVDV1_1-385, BVDV1_29-391, CrPV_5NCR, CrPV_IGR, crTMV_IREScp, crTMV_IRESmp75, crTMV_IRESmp228, crTMV_IREScp, crTMV_IREScp, CSFV, CVB3, DCV_IGR, EMCV-R, EoPV_5NTR, ERAV 245-961, ERBV 162-920, EV71_1-748, FeLV-Notch2, FMDV_type_C, GBV-A, GBV-B, GBV-C, gypsy_env, gypsyD5, gypsyD2, HAV_HM175, HCV type_1a, HiPV_IGRpred, HIV-1, HoCV1_IGRpred, HRV-2, IAPV_IGRpred, idefix, KBV_IGRpred, LINE-1_ORF1_-101_to_-1, LINE-1_ORF1-302_to_-202, LINE-1_ORF2-138_to_-86, LINE-1_ORF1_-44to_-1, PSIV_IGR, PV typel_Mahoney, PV_type3_Leon, REV-A, RhPV_5NCR, RhPV_IGR, SINV1_IGRpred, SV40_661-830, TMEV, TMV_UI_IRESmp228, TRV_5NTR, TrV_IGR, or TSV_IGR. In some embodiments, the IRES element is at least partially derived from a cellular IRES, such as AML1/RUNX1, Antp-D, Antp-DE, Antp-CDE, Apaf-1, Apaf-1, AQP4, AT1R_varl, AT1R_var2, AT1R_var3, AT1R_var4, BAG1_p36delta236 nt, BAG1_p36, BCL2, BiP_-222_-3, c-IAP1_285-1399, c-IAP1_1313-1462, c-jun, c-myc, Cat-1224, CCND1, DAPS, eIF4G, eIF4GI-ext, eIF4GII, eIF4GII-long, ELG1, ELH, FGF1A, FMR1, Gtx-133-141, Gtx-1-166, Gtx-1-120, Gtx-1-196, hairless, HAP4, HIFla, hSNM1, Hsp101, hsp70, hsp70, Hsp90, IGF2_leader2, Kv1.4_1.2, L-myc, LamB1_-335_-1, LEF1, MNT_75-267, MNT_36-160, MTG8a, MYB, MYT2_997-1152, n-MYC, NDST1, NDST2, NDST3, NDST4L, NDST4S, NRF_-653_-17, NtHSF1, ODC1, p27kipl, 03_128-269, PDGF2/c-sis, Pim-1, PITSLRE_p58, Rbm3, reaper, Scamper, TFIID, TIF4631, Ubx_1-966, Ubx_373-961, UNR, Ure2, UtrA, VEGF-A-133-1, XIAP_5-464, XIAP_305-466, or YAP1.

xxv. Termination Element

In some embodiments, the oRNA includes one or more cargo or payload sequences (also referred to as expression sequences) and each cargo or payload sequence may or may not have a termination element.

In some embodiments, the oRNA includes one or more cargo or payload sequences and the sequences lack a termination element, such that the oRNA is continuously translated. Exclusion of a termination element may result in rolling circle translation or continuous expression of the encoded peptides or polypeptides as the ribosome will not stalling or fall-off. In such an embodiment, rolling circle translation expresses a continuous expression through each cargo or payload sequence.

In some embodiments, one or more cargo or payload sequences in the oRNA comprise a termination element.

In some embodiments, not all of the cargo or payload sequences in the oRNA comprise a termination element. In such instances, the cargo or payload may fall off the ribosome when the ribosome encounters the termination element and terminates translation. In some embodiments, translation is terminated while at least one region of the ribosome remains in contact with the oRNA.

xxvi. Rolling Circle Translation

In some embodiments, once translation of the oRNA is initiated, the ribosome bound to the oRNA does not disengage from the oRNA before finishing at least one round of translation of the oRNA. In some embodiments, the oRNA as described herein is competent for rolling circle translation. In some embodiments, during rolling circle translation, once translation of the oRNA is initiated, the ribosome bound to the oRNA does not disengage from the oRNA before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 100,000 rounds, or at least 1,000,000 rounds of translation of the oRNA.

In some embodiments, the rolling circle translation of the oRNA leads to generation of polypeptide that is translated from more than one round of translation of the oRNA. In some embodiments, the oRNA comprises a stagger element, and rolling circle translation of the oRNA leads to generation of polypeptide product that is generated from a single round of translation or less than a single round of translation of the oRNA.

xxvii. Circularization

In one embodiment, a linear RNA may be cyclized, or concatemerized. In some embodiments, the linear RNA may be cyclized in vitro prior to formulation and/or delivery. In some embodiments, the linear RNA may be cyclized within a cell.

In some embodiments, the mechanism of cyclization or concatemerization may occur through at least 3 different routes: 1) chemical, 2) enzymatic, and 3) ribozyme catalyzed. The newly formed 5′-/3′-linkage may be intramolecular or intermolecular.

In the first route, the 5′-end and the 3′-end of the nucleic acid contain chemically reactive groups that, when close together, form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a synthetic mRNA molecule will undergo a nucleophilic attack on the 5′-NHS-ester moiety forming a new 5′-/3′-amide bond.

In the second route, T4 RNA ligase may be used to enzymatically link a 5′-phosphorylated nucleic acid molecule to the 3′-hydroxyl group of a nucleic acid forming a new phosphodiester linkage. In an example reaction, 10 ng of a nucleic acid molecule is incubated at 37° C. for 1 hour with 1-10 units of T4 RNA ligase (New England Biolabs, Ipswich, MA) according to the manufacturer's protocol. The ligation reaction may occur in the presence of a split oligonucleotide capable of base-pairing with both the 5′- and 3′-region in juxtaposition to assist the enzymatic ligation reaction.

In the third route, either the 5′- or 3′-end of the cDNA template encodes a ligase ribozyme sequence such that during in vitro transcription, the resultant nucleic acid molecule can contain an active ribozyme sequence capable of ligating the 5′-end of a nucleic acid molecule to the 3′-end of a nucleic acid molecule. The ligase ribozyme may be derived from the Group I Intron, Group II Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

xxviii. Extracellular Circularization

In some embodiments, the linear RNA is cyclized, or concatemerized using a chemical method to form an oRNA. In some chemical methods, the 5′-end and the 3′-end of the nucleic acid (e.g., a linear RNA) include chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA will undergo a nucleophilic attack on the 5′-NHS-ester moiety forming a new 5′-/3′-amide bond.

In one embodiment, a DNA or RNA ligase may be used to enzymatically link a 5′-phosphorylated nucleic acid molecule (e.g., a linear RNA) to the 3′-hydroxyl group of a nucleic acid (e.g., a linear nucleic acid) forming a new phosphorodiester linkage. In an example reaction, a linear RNA is incubated at 37C for 1 hour with 1-10 units of T4 RNA ligase according to the manufacturer's protocol. The ligation reaction may occur in the presence of a linear nucleic acid capable of base-pairing with both the 5′- and 3′-region in juxtaposition to assist the enzymatic ligation reaction. In one embodiment, the ligation is splint ligation where a single stranded polynucleotide (splint), like a single stranded RNA, can be designed to hybridize with both termini of a linear RNA, so that the two termini can be juxtaposed upon hybridization with the single-stranded splint. Splint ligase can thus catalyze the ligation of the juxtaposed two termini of the linear RNA, generating an oRNA.

In one embodiment, a DNA or RNA ligase may be used in the synthesis of the oRNA. As a non-limiting example, the ligase may be a circ ligase or circular ligase.

In one embodiment, either the 5′- or 3′-end of the linear RNA can encode a ligase ribozyme sequence such that during in vitro transcription, the resultant linear RNA includes an active ribozyme sequence capable of ligating the 5′-end of the linear RNA to the 3′-end of the linear RNA. The ligase ribozyme may be derived from the Group I Intron, Hepatitis Delta Virus, Hairpin ribozyme or may be selected by SELEX (systematic evolution of ligands by exponential enrichment).

In one embodiment, a linear RNA may be cyclized or concatemerized by using at least one non-nucleic acid moiety. In one aspect, the at least one non-nucleic acid moiety may react with regions or features near the 5′ terminus and/or near the 3′ terminus of the linear RNA in order to cyclize or concatermerize the linear RNA. In another aspect, the at least one non-nucleic acid moiety may be located in or linked to or near the 5′ terminus and/or the 3′ terminus of the linear RNA. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In one embodiment, a linear RNA may be cyclized or concatemerized due to a non-nucleic acid moiety that causes an attraction between atoms, molecular surfaces at, near or linked to the 5′ and 3′ ends of the linear RNA. As a non-limiting example, one or more linear RNA may be cyclized or concatemerized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agostic bonds, dipolar bonds, conjugation, hyperconjugation and antibonding.

In one embodiment, the linear RNA may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′ terminus may associate with each other causing a linear RNA to cyclize or concatemerize. In another aspect, the peptides covalently linked to the ribozyme RNA near the 5′ terminus and the 3′ terminus may cause the linear RNA to cyclize or concatemerize after being subjected to ligation using various methods known in the art such as, but not limited to, protein ligation.

In some embodiments, the linear RNA may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear RNA into a 5′ monophosphate may occur by a two-step reaction comprising: (a) contacting the 5′ nucleotide of the linear RNA with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

xxix. Splicing Element

In some embodiment, the oRNA includes at least one splicing element. The splicing element can be a complete splicing element that can mediate splicing of the oRNA or the spicing element can be a residual splicing element from a completed splicing event. For instance, in some cases, a splicing element of a linear RNA can mediate a splicing event that results in circularization of the linear RNA, thereby the resultant oRNA comprises a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the oRNA includes a splicing element adjacent each expression sequence. In some embodiments, the splicing element is on one or both sides of each expression sequence, leading to separation of the expression products, e.g., peptide(s) and or polypeptide(s).

In some embodiments, the oRNA includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in cis-sequence elements proximal to backsplice events such as sequences in the 200 bp preceding (upstream of) or following (downstream from) a backsplice site with flanking exons. In some embodiments, the oRNA includes at least one repetitive nucleotide sequence described elsewhere herein as an internal splicing element. In such embodiments, the repetitive nucleotide sequence may include repeated sequences from the Alu family of introns.

In some embodiments, the oRNA may include canonical splice sites that flank head-to-tail junctions of the oRNA.

In some embodiments, the oRNA may include a bulge-helix-bulge motif, comprising a 4-base pair stem flanked by two 3-nucleotide bulges. Cleavage occurs at a site in the bulge region, generating characteristic fragments with terminal 5′-hydroxyl group and 2′, 3′-cyclic phosphate. Circularization proceeds by nucleophilic attack of the 5′—OH group onto the 2′, 3′-cyclic phosphate of the same molecule forming a 3′, 5′-phosphodiester bridge.

In some embodiments, the oRNA may include a sequence that mediates self-ligation. Non-limiting examples of sequences that can mediate self-ligation include a self-circularizing intron, e.g., a 5′ and 3′ slice junction, or a self-circularizing catalytic intron such as a Group I, Group II or Group III Introns. Non-limiting examples of group I intron self-splicing sequences may include self-splicing permuted intron-exon sequences derived from T4 bacteriophage gene thymidylate synthase, and the intervening sequence (IVS) rRNA of Tetrahymena.

xxx. Other Circularization Methods

In some embodiments, linear RNA may include complementary sequences, including either repetitive or nonrepetitive nucleic acid sequences within individual introns or across flanking introns. In some embodiments, the oRNA includes a repetitive nucleic acid sequence. In some embodiments, the repetitive nucleotide sequence includes poly CA or poly UG sequences. In some embodiments, the oRNA includes at least one repetitive nucleic acid sequence that hybridizes to a complementary repetitive nucleic acid sequence in another segment of the oRNA, with the hybridized segment forming an internal double strand. In some embodiments, repetitive nucleic acid sequences and complementary repetitive nucleic acid sequences from two separate oRNA that hybridize to generate a single oRNA, with the hybridized segments forming internal double strands. In some embodiments, the complementary sequences are found at the 5′ and 3′ ends of the linear RNA. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the oRNA. Such methods may include, but are not limited to click chemistry (e.g., alkyne and azide based methods, or clickable bases), olefin metathesis, phosphoramidate ligation, hemiaminal-imine crosslinking, base modification, and any combination thereof.

In some embodiments, enzymatic methods of circularization may be used to generate the oRNA. In some embodiments, a ligation enzyme, e.g., DNA or RNA ligase, may be used to generate a template of the oRNA or complement, a complementary strand of the oRNA, or the oRNA.

xxxi. Ribosomal RNA (rRNA)

Ribosomal RNAs (rRNAs) are RNA which form ribosomes. Ribosomes are essential to protein synthesis and contain a large and small ribosomal subunit. In prokaryotes, a small 30S and large 50S ribosomal subunit make up a 70S ribosome. In eukaryotes, the 40S and 60S subunit form an 80S ribosome. In order to bind aminoacyl-tRNAs and link amino acids together to create polypeptides, the ribosome contains 3 sites: an exit site (E), a peptidyl site (P), and acceptor site (A).

In some embodiments, the originator construct and/or the benchmark construct comprises or encodes a rRNA.

xxxii. Long Non-Coding RNA (lncRNA)

Long non-coding RNAs (lncRNAs) are regulatory RNA molecules that do not code for proteins but influence a vast array of biological processes. The lncRNA designation is generally restricted to non-coding transcripts longer than about 200 nucleotides. The length designation differentiates lncRNA from small regulatory RNAs such as short interfering RNA (siRNA) and micro RNA (miRNA). In vertebrates, the number of lncRNA species is thought to greatly exceed the number of protein-coding species. It is also thought that lncRNAs drive biologic complexity observed in vertebrates compared to invertebrates. Evidence of this complexity is seen in many cellular compartments of a vertebrate organism such as the T lymphocyte compartment of the adaptive immune system. Differences in expression and function of lncRNA can be major contributors to human disease.

In some embodiments, the originator constructs and/or the benchmark constructs comprise lncRNAs.

xxxiii. RNA Modifications

In some aspects, the originator constructs or benchmark constructs may contain one or more modified nucleotides such as, but not limited to, sugar modified nucleotides, nucleobase modifications and/or backbone modifications. In some aspects, the originator constructs or benchmark constructs may contain combined modifications, for example, combined nucleobase and backbone modifications.

In some embodiments, the modified nucleotide may be a sugar-modified nucleotide. Sugar modified nucleotides include, but are not limited to 2′-fluoro, 2′-amino and 2′-thio modified ribonucleotides, e.g. 2′-fluoro modified ribonucleotides. Modified nucleotides may be modified on the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

In some embodiments, the modified nucleotide may be a nucleobase-modified nucleotide.

In some embodiments, the modified nucleotide may be a backbone-modified nucleotide. In some embodiments, the originator constructs or benchmark constructs may further comprise other modifications on the backbone. A normal “backbone”, as used herein, refers to the repeating alternating sugar-phosphate sequences in a DNA or RNA molecule. The deoxyribose/ribose sugars are joined at both the 3-hydroxyl and 5′-hydroxyl groups to phosphate groups in ester links, also known as “phosphodiester” bonds/linker (PO linkage). The PO backbones may be modified as “phosphorothioate backbone (PS linkage). In some cases, the natural phosphodiester bonds may be replaced by amide bonds but the four atoms between two sugar units are kept. Such amide modifications can facilitate the solid phase synthesis of oligonucleotides and increase the thermodynamic stability of a duplex formed with siRNA complement.

Modified bases refer to nucleotide bases such as, but not limited to, adenine, guanine, cytosine, thymine, uracil, xanthine, inosine, and queuosine that have been modified by the replacement or addition of one or more atoms or groups. Some examples of modifications on the nucleobase moieties include, but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

The originator constructs and/or benchmark constructs may include one or more substitutions, insertions and/or additions, deletions, and covalent modifications with respect to reference sequences, in particular, the parent RNA, are included within the scope of this invention.

In some embodiments, the originator constructs and/or benchmark constructs includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, and the like). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the modified nucleic acid comprises messenger RNA (mRNA). In some embodiments, the originator constructs and/or benchmark constructs comprise at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The originator constructs and/or benchmark constructs may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof. Additional modifications are described herein.

In some embodiments, the originator constructs and/or benchmark constructs includes at least one N(6)methyladenosine (m6A) modification to increase translation efficiency. In some embodiments, the N(6)methyladenosine (m6A) modification can reduce immunogeneicity of the originator constructs and/or benchmark constructs.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the RNA may enhance immune evasion. The RNA may be synthesized and/or modified by methods well established in the art, such as those described in “Current protocols in nucleic acid chemistry,” Beaucage, S. L. et al. (Eds.), John Wiley & Sons, Inc., New York, N.Y., USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases may also include 5-methylcytidine and pseudouridine. In some embodiments, base modifications may modulate expression, immune response, stability, subcellular localization, to name a few functional effects, of the RNA. In some embodiments, the modification includes a bi-orthogonal nucleotides, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more RNA may, as well as backbone modifications, include modification or replacement of the phosphodiester linkages. Specific examples of modifications include modified backbones or no natural internucleoside linkages such as internucleoside modifications, including modification or replacement of the phosphodiester linkages. RNA having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, the RNA will include ribonucleotides with a phosphorus atom in its internucleoside backbone.

Modified RNA backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the RNA may be negatively or positively charged.

The modified nucleotides can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the RNA is expected to reduce the innate immune response through weaker binding/activation of cellular innate immune molecules.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the RNA may include one or more cytotoxic nucleosides. For example, cytotoxic nucleosides may be incorporated into RNA, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the RNA sequence includes or comprises natural nucleosides (e.g., adenosine, guanosine, cytidine, uridine), nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), and/or modified phosphate groups (e.g., phosphorothioates and 5′-N-phosphoramidite linkages). In one embodiment, the RNA sequence includes or comprises incorporates pseudouridine (y). In another embodiment, the RNA sequence includes or comprises 5-methylcytosine (m5C).

The RNA may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the RNA, or in a given predetermined sequence region thereof. In some embodiments, the RNA includes a pseudouridine. In some embodiments, the RNA includes an inosine, which may aid in the immune system characterizing the RNA as endogenous versus viral RNAs. The incorporation of inosine may also mediate improved RNA stability/reduced degradation.

In some embodiments, all nucleotides in the RNA (or in a given sequence region thereof) are modified. In some embodiments, the modification may include an m6A, which may augment expression, an inosine, which may attenuate an immune response, pseudouridine, which may increase RNA stability, or translational readthrough (stagger element), an m5C, which may increase stability, and a 2,2,7-trimethylguanosine, which aids subcellular translocation (e.g., nuclear localization).

Different sugar modifications, nucleotide modifications, and/or internucleoside linkages (e.g., backbone structures) may exist at various positions in the RNA. One of ordinary skill in the art will appreciate that the nucleotide analogs or other modification(s) may be located at any position(s) of the RNA, such that the function of the RNA is not substantially decreased. A modification may also be a non-coding region modification. The RNA may include from about 1% to about 100% modified nucleotides (either in relation to overall nucleotide content, or in relation to one or more types of nucleotide, e.g., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

xxxiv. Codon Optimization

A nucleotide sequence of the originator construct and/or benchmark construct may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the mRNA. Codon optimization tools, algorithms and services are known in the art, non-limiting examples include services from GeneArt (Life Technologies), DNA2.0 (Menlo Park CA) and/or proprietary methods. In some embodiments, the ORF sequence is optimized using optimization algorithms.

III. DELIVERY VEHICLES AND TRACKING SYSTEMS

Originator constructs and benchmark constructs described herein may be formulated in a delivery vehicle (e.g., a pharmaceutical delivery vehicle). Non-limiting examples of pharmaceutical delivery vehicles include lipid nanoparticles, non-lipid nanoparticles, exosomes, liposomes, micelles, viral particles, and polymeric delivery technology.

In some embodiments, the delivery vehicle comprises an originator construct or a benchmark construct with at least one cargo or payload. The cargo or payload may be any DNA, RNA or polypeptide described herein.

In some embodiments, the delivery vehicle comprises an originator construct or a benchmark construct with at least one cargo or payload which is a coding RNA.

In some embodiments, the delivery vehicle comprises an originator construct or a benchmark construct with at least one cargo or payload which is a non-coding RNA.

In some embodiments, the delivery vehicle comprises an originator construct or a benchmark construct with at least one cargo or payload which is a oRNA.

In some embodiments, the delivery vehicle comprises an originator construct or a benchmark construct with at least one cargo or payload which is a siRNA.

In some embodiments, the delivery vehicle comprises an originator construct or a benchmark construct with at least one cargo or payload which is an mRNA.

In some embodiments, the at least one RNA compound is comprised of a functional RNA where the RNA results in at least one change in a cell, tissue, organ and/or organism. Said changes in state may include, but are not limited to, altering the expression level of a polypeptide, altering the translation level of a nucleic acid, altering the expression level of a nucleic acid, altering the amount of a polypeptide present in a cell, tissue, organ and/or organism, changing a genetic sequence of a cell, tissue, organ and/or organism, adding nucleic acids to a target genome, subtracting nucleic acids from a target genome, altering physiological activity in a cell, tissue, organ and/or organism or any combination thereof.

In some embodiments, the delivery vehicle comprises an originator construct or a benchmark construct with at least one cargo or payload which is DNA.

In some embodiments, the delivery vehicle comprises an originator construct or a benchmark construct with two cargos or payloads which are DNA. The DNA may be the same DNA or different DNA. As a non-limiting example, the DNA are the same. As a non-limiting example, the DNA are different. As a non-limiting example, the DNA are different but encode the same payload or cargo. As a non-limiting example, the DNA are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with three cargos or payloads which are DNA. The DNA may be the same DNA or different DNA. As a non-limiting example, the DNA are the same. As a non-limiting example, the DNA are different. As a non-limiting example, two DNA are the same and one is different. As a non-limiting example, the first DNA is different from the second and third DNA. As a non-limiting example, the first DNA, second DNA and third DNA are all different. As a non-limiting example, the first DNA is different from the second and third DNA but they all encode the same payload or cargo. As a non-limiting example, the first DNA is different from the second and third DNA but the second and third DNA encode the same payload or cargo.

In some embodiments, the delivery vehicle comprises an originator construct or a benchmark construct with at least one cargo or payload which is a polypeptide.

In some embodiments, the delivery vehicle comprises an originator construct or a benchmark construct with two cargos or payloads which are polypeptide. The polypeptide may be the same polypeptide or different polypeptide As a non-limiting example, the polypeptide are the same. As a non-limiting example, the polypeptide are different. As a non-limiting example, the polypeptides are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with three cargos or payloads which are polypeptide. The polypeptide may be the same polypeptide or different polypeptide. As a non-limiting example, the polypeptide are the same. As a non-limiting example, the polypeptide are different. As a non-limiting example, two polypeptide are the same and one is different. As a non-limiting example, the first polypeptide is different from the second and third polypeptide. As a non-limiting example, the first polypeptide, second polypeptide and third polypeptide are all different. As a non-limiting example, the first polypeptide is different from the second and third polypeptide but they all encode the same payload or cargo. As a non-limiting example, the first polypeptide is different from the second and third polypeptide but the second and third polypeptide encode the same payload or cargo.

In some embodiments, the delivery vehicle comprises an originator construct or a benchmark construct with at least one cargo or payload which is a peptide.

In some embodiments, the delivery vehicle comprises an originator construct or a benchmark construct with two cargos or payloads which are peptide. The peptide may be the same peptide or different peptide As a non-limiting example, the peptide are the same. As a non-limiting example, the peptide are different. As a non-limiting example, the peptides are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with three cargos or payloads which are peptide. The peptide may be the same peptide or different peptide. As a non-limiting example, the peptide are the same. As a non-limiting example, the peptide are different. As a non-limiting example, two peptide are the same and one is different. As a non-limiting example, the first peptide is different from the second and third peptide. As a non-limiting example, the first peptide, second peptide and third peptide are all different. As a non-limiting example, the first peptide is different from the second and third peptide but they all encode the same payload or cargo. As a non-limiting example, the first peptide is different from the second and third peptide but the second and third peptide encode the same payload or cargo.

In some embodiments, the delivery vehicle comprises an originator construct or a benchmark construct with at least one cargo or payload which is RNA.

In some embodiments, the delivery vehicle comprises an originator construct or a benchmark construct with two cargos or payloads which are RNA. The RNA may be the same RNA or different RNA. As a non-limiting example, the RNA are the same. As a non-limiting example, the RNA are different. As a non-limiting example, the RNA are different but encode the same payload or cargo. As a non-limiting example, the RNA are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with three cargos or payloads which are RNA. The RNA may be the same RNA or different RNA. As a non-limiting example, the RNA are the same. As a non-limiting example, the RNA are different. As a non-limiting example, two RNA are the same and one is different. As a non-limiting example, the first RNA is different from the second and third RNA. As a non-limiting example, the first RNA, second RNA and third RNA are all different. As a non-limiting example, the first RNA is different from the second and third RNA but they all encode the same payload or cargo. As a non-limiting example, the first RNA is different from the second and third RNA but the second and third RNA encode the same payload or cargo.

In some embodiments, the delivery vehicle comprises an originator construct or a benchmark construct with two cargos or payloads where one is RNA and one is DNA. The RNA and DNA may encode the same peptide or polypeptide or may encode different peptides or polypeptides. As a non-limiting example, the RNA and DNA may encode the same peptide or polypeptide. As a non-limiting example, the RNA and DNA may encode different peptides or polypeptides. As a non-limiting example, the RNA and DNA are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with two cargos or payloads where one is RNA and one is a peptide. The RNA may encode the same peptide as the peptide cargo/payload the RNA may encode a different peptide. As a non-limiting example, the RNA encodes the same peptide. As a non-limiting example, the RNA encodes a different peptides. As a non-limiting example, the RNA and peptide are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with two cargos or payloads where one is RNA and one is a polypeptide. The RNA may encode the same polypeptide as the polypeptide cargo/payload the RNA may encode a different polypeptide. As a non-limiting example, the RNA encodes the same polypeptide. As a non-limiting example, the RNA encodes a different polypeptides. As a non-limiting example, the RNA and polypeptide are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with two cargos or payloads where one is DNA and one is a peptide. The DNA may encode the same peptide as the peptide cargo/payload the DNA may encode a different peptide. As a non-limiting example, the DNA encodes the same peptide. As a non-limiting example, the DNA encodes a different peptides. As a non-limiting example, the DNA and peptide are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with two cargos or payloads where one is DNA and one is a polypeptide. The DNA may encode the same polypeptide as the polypeptide cargo/payload the DNA may encode a different polypeptide. As a non-limiting example, the DNA encodes the same polypeptide. As a non-limiting example, the DNA encodes a different polypeptides. As a non-limiting example, the DNA and polypeptide are different pieces of a larger payload or cargo (e.g., heavy chain or light chain of an antibody) that can come together using natural systems or synthetic methods known in the art to produce a functional polypeptide (e.g., antibody).

In some embodiments, the target organ, tissue, or cell is not the liver.

In some embodiments, the target organ, tissue, or cell is not the kidney.

In some embodiments, the target organ, tissue, or cell is not the spleen.

In some embodiments, a higher proportion of the polynucleotides are delivered to a target organ, tissue, or cell than to the liver following administration (e.g., intravenous administration) to a subject.

In some embodiments, a higher proportion of the polynucleotides are delivered to a target organ, tissue, or cell than to the kidney following administration (e.g., intravenous administration) to a subject.

In some embodiments, a higher proportion of the polynucleotides are delivered to a target organ, tissue, or cell than to the spleen following administration (e.g., intravenous administration) to a subject.

In some embodiments, 1.5 times, 2 times, 3 times, 4, times, 5 times, 6 times, 7 times, 8 times, 9 times, 10 times, 20 times, 30 times, 40 times, 50 times, 60 times, 70 times, 80 times, 90 times, 100 times or more of the polynucleotides are delivered to a target organ, tissue, or cell than to the liver following administration (e.g., intravenous administration) to a subject. For example, the amount of polynucleotides delivered to a target organ, tissue, or cell can be at least about 50%, at least about 60%, at least about 70%, at least about 80%, at least about 90%, at least about 100%, at least about 120%, at least about 140%, at least about 160%, at least about 180%, at least about 200%, at least about 240%, at least about 240%, at least about 260%, or at least about 280% the amount of polynucleotides delivered to the liver following administration (e.g., intravenous administration). In some embodiments, 1.5 times, 2 times, 3 times, 4, times, 5 times, 6 times, 7 times, 8 times, 9 times, 10 times, 20 times, 30 times, 40 times, 50 times, 60 times, 70 times, 80 times, 90 times, 100 times or more of the polynucleotides are delivered to a target organ, tissue, or cell than to the liver following administration (e.g., intravenous administration) to a subject, when accounting for relative weight of the target organ, tissue, or cell as compared to the liver. In some embodiments, 1.5 times, 2 times, 3 times, 4, times, 5 times, 6 times, 7 times, 8 times, 9 times, 10 times, 20 times, 30 times, 40 times, 50 times, 60 times, 70 times, 80 times, 90 times, 100 times or greater concentration of the polynucleotides are delivered to the target organ, tissue or cell than to the liver following administration (e.g., intravenous administration) to a subject.

In some embodiments, 1.5 times, 2 times, 3 times, 4, times, 5 times, 6 times, 7 times, 8 times, 9 times, 10 times, 20 times, 30 times, 40 times, 50 times, 60 times, 70 times, 80 times, 90 times, 100 times or more of the polynucleotides are delivered to a target organ, tissue, or cell than to the kidney following administration (e.g., intravenous administration) to a subject. For example, the amount of polynucleotides delivered to a target organ, tissue, or cell can be at least about 50%, at least about 60%, at least about 70%, at least about 80%, at least about 90%, at least about 100%, at least about 120%, at least about 140%, at least about 160%, at least about 180%, at least about 200%, at least about 240%, at least about 240%, at least about 260%, or at least about 280% the amount of polynucleotides delivered to the kidney following administration (e.g., intravenous administration). In some embodiments, about 1.5 times, about 2 times, about 3 times, about 4, times, about 5 times, about 6 times, about 7 times, about 8 times, about 9 times, about 10 times, about 20 times, about 30 times, about 40 times, about 50 times, about 60 times, about 70 times, about 80 times, about 90 times, about 100 times or more of the polynucleotides are delivered to a target organ, tissue, or cell than to the kidney following administration (e.g., intravenous administration) to a subject, when accounting for relative weight of the target organ, tissue, or cell as compared to the kidney. In some embodiments, about 1.5 times, about 2 times, about 3 times, about 4 times, about 5 times, about 6 times, about 7 times, about 8 times, about 9 times, about 10 times, about 20 times, about 30 times, about 40 times, about 50 times, about 60 times, about 70 times, about 80 times, about 90 times, about 100 times or greater concentration of the polynucleotides are delivered to the target organ, tissue or cell than to the kidney following administration (e.g., intravenous administration) to a subject.

In some embodiments, 1.5 times, 2 times, 3 times, 4, times, 5 times, 6 times, 7 times, 8 times, 9 times, 10 times, 20 times, 30 times, 40 times, 50 times, 60 times, 70 times, 80 times, 90 times, 100 times or more of the polynucleotides are delivered to a target organ, tissue, or cell than to the spleen following administration (e.g., intravenous administration) to a subject. For example, the amount of polynucleotides delivered to a target organ, tissue, or cell can be at least about 50%, at least about 60%, at least about 70%, at least about 80%, at least about 90%, at least about 100%, at least about 120%, at least about 140%, at least about 160%, at least about 180%, at least about 200%, at least about 240%, at least about 240%, at least about 260%, or at least about 280% the amount of polynucleotides delivered to the spleen following administration (e.g., intravenous administration).In some embodiments, about 1.5 times, about 2 times, about 3 times, about 4 times, about 5 times, about 6 times, about 7 times, about 8 times, about 9 times, about 10 times, about 20 times, about times, about 40 times, about 50 times, about 60 times, about 70 times, about 80 times, about 90 times, about 100 times or more of the polynucleotides are delivered to a target organ, tissue, or cell than to the spleen following administration (e.g., intravenous administration) to a subject, when accounting for relative weight of the target organ, tissue, or cell as compared to the spleen. In some embodiments, about 1.5 times, about 2 times, about 3 times, about 4 times, about 5 times, about 6 times, about 7 times, about 8 times, about 9 times, about 10 times, about 20 times, about 30 times, about 40 times, about 50 times, about 60 times, about 70 times, about 80 times, about 90 times, about 100 times or greater concentration of the polynucleotides are delivered to the target organ, tissue or cell than to the spleen following administration (e.g., intravenous administration) to a subject.

a. Delivery Vehicles

i. Nanoparticles

In some embodiments, the delivery vehicle is a nanoparticle. The term “nanoparticle” as used herein refers to any particle ranging in size from 10-1000 nm. The nanoparticle may be 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 nm.

ii. Lipid Nanoparticles

In some embodiments, the nanoparticles may be a lipid nanoparticle (LNP). In general, LNPs can be characterized as small solid or semi-solid particles possessing an exterior lipid layer with a hydrophilic exterior surface that is exposed to the non-LNP environment, an interior space which may aqueous (vesicle like) or non-aqueous (micelle like), and at least one hydrophobic inter-membrane space. LNP membranes may be lamellar or non-lamellar and may be comprised of 1, 2, 3, 4, 5 or more layers. In some embodiments, LNPs may comprise a cargo or a payload into their interior space, into the inter membrane space, onto their exterior surface, or any combination thereof.

LNPs useful herein are known in the art and generally comprise cholesterol (aids in stability and promotes membrane fusion), a phospholipid (which provides structure to the LNP bilayer and also may aid in endosomal escape), a polyethylene glycol (PEG) derivative (which reduces LNP aggregation and “shields” the LNP from non-specific endocytosis by immune cells), and an ionizable lipid (complexes negatively charged RNA and enhances endosomal escape), which form the LNP-forming composition.

The components of the LNP may be selected based on the desired target, cargo, size, etc. As a non-limiting example, previous studies have shown that that polymeric nanoparticles made of low molecular weight polyamines and lipids can deliver nucleic acids to endothelial cells with high efficiency. (Dahlman, et al, In vivo endothelial siRNA delivery using polymeric nanoparticles with low molecular weight, Nat Nanotechnol. 2014 August; 9(8): 648-655; the contents of which is herein incorporated by reference in its entirety).

In some embodiments, the originator constructs and benchmark constructs of the present disclosure may be incorporated into lipid nanoparticles (LNPs). In some embodiments a lipid nanoparticle may be comprised of at least one cationic lipid, at least one non-cationic lipid, at least one sterol, at least one particle-activity-modifying-agent, or any combination thereof. In some embodiments a lipid nanoparticle may be comprised of at least one cationic lipid, at least one non-cationic lipid, at least one sterol, and at least one particle-activity-modifying-agent. In some embodiments, the LNP may be comprised of at least one cationic lipid, at least one non-cationic lipid, and at least one sterol. In some embodiments, the LNP may be comprised of at least one cationic lipid, at least one non-cationic lipid, and at least one particle-activity-modifying-agent. In some embodiments, the LNP may be comprised of at least one non-cationic lipid, at least one sterol, and at least one particle-activity-modifying-agent. In some embodiments, the LNP may be comprised of at least one cationic lipid and at least one non-cationic lipid. In some embodiments, the LNP may be comprised of at least one cationic lipid and at least one sterol. In some embodiments, the LNP may be comprised of at least one cationic lipid and at least one particle-activity-modifying-agent. In some embodiments, the LNP may be comprised of at least one non-cationic lipid and at least one sterol. In some embodiments, the LNP may be comprised of at least one non-cationic lipid and at least one particle-activity-modifying-agent. In some embodiments, the LNP may be comprised of at least one sterol and at least one particle-activity-modifying-agent. In some embodiments, the LNP may be comprised of at least one cationic lipid. In some embodiments, the LNP may be comprised of at least one non-cationic lipid. In some embodiments, a LNP may be comprised of a sterol. In some embodiments, the LNP may be comprised of a particle-activity-modifying-agent.

In some embodiments, the at least one cationic lipid may comprise any of at least one ionizable cationic lipid, at least one amino lipid, at least one saturated cationic lipid, at least one unsaturated cationic lipid, at least one zwitterionic lipid, at least one multivalent cationic lipid, or any combination thereof. In some embodiments, the LNP may be essentially devoid of the at least one cationic lipid. In some embodiments, the LNP may contain no amount of the at least one cationic lipid.

In some embodiments, at least one cationic lipid may be selected from, but not limited to, at least one of 1,3-Bis-(1,2-bis-tetradecyloxy-propyl-3-dimethylethoxyammoniumbromide)-propan-2-ol ((R)-PLC-2), 2-(Dinonylamino)ethan-1-ol (17-10), 2-(Didodecylamino)ethan-1-ol (17-11), 3-(Didodecylamino)propan-1-ol (17-12), 4-(Didodecylamino)butan-1-ol (17-13), 2-(Hexyl((9Z,12Z)-octadeca-9,12-dien-1-yl)amino)ethan-1-ol (17-2), 2-(Nonyl((9Z,12Z)-octadeca-9,12-dien-1-yl)amino)ethan-1-ol (17-3), 2-(Dodecyl((9Z,12Z)-octadeca-9,12-dien-1-yl)amino)ethan-1-ol (17-4), 2-(((9Z,12Z)-Octadeca-9,12-dien-1-yl)(tetradecyl)amino)ethan-1-ol (17-5), 2-(((9Z,12Z)-Octadeca-9,12-dien-1-yl)(octadecyl)amino)ethan-1-ol (17-6), 2-(Ditetradecylamino)ethan-1-ol (17-7), 2-(Di((Z)-octadec-9-en-1-yl)amino)ethan-1-ol (17-8), (9Z,12Z)—N-(2-Methoxyethyl)-N-((9Z,12Z)-octadeca-9,12-dien-1-yl)octadeca-9,12-dien-1-amine (17-9), N-Nonyl-N-(2-(piperazin-1-yl)ethyl)nonan-1-amine (19-1), N-Dodecyl-N-(2-(piperazin-1-yl)ethyl)dodecan-1-amine (19-2), (9Z,12Z)—N-((9Z,12Z)-Octadeca-9,12-dien-1-yl)-N-(2-(piperazin-1-yl)ethyl)octadeca-9,12-dien-1-amine (19-3), N-Dodecyl-N-(2-(4-methylpiperazin-1-yl)ethyl)dodecan-1-aminelntermediatel:2-(Didodecylamino)ethan-1-ol (19-4), N-Dodecyl-N-(2-(4-(4-methoxybenzyl)piperazin-1-yl)ethyl)dodecan-1-amine (19-5), (9Z,12Z)—N-(2-(4-Dodecylpiperazin-1-yl)ethyl)-N-((9Z,12Z)-octadeca-9,12-dien-1-yl)octadeca-9,12-dien-1-amine (19-6), (3-((6Z,9Z,28Z,31Z)-heptatriaconta-6,9,28,31-tetraen-19-yloxy)-N,N-dimethylpropan-1-amine) (1-B1 1), N-(2-(Didodecylamino)ethyl)-N-dodecylglycine (20-1), Dinonyl8,8′-((2-(dodecyl(2-hydroxyethyl)amino)ethyl)azanediyl)dioctanoate (20-10), 3-((2-(Ditetradecylamino)ethyl)(dodecyl)amino)propan-1-ol (20-11), 2-((2-(Ditetradecylamino)ethyl)(tetradecyl)amino)ethan-1-ol (20-12), 2-((2-(Di((9Z,12Z)-octadeca-9,12-dien-1-yl)amino)ethyl)(dodecyl)amino)ethan-1-ol (20-13), 2-((2-(Di((9Z,12Z)-octadeca-9,12-dien-1-yl)amino)ethyl)((9Z,12Z)-octadeca-9,12-dien-1-yl)amino)ethan-1-ol (20-14), 2-((2-(Didodecylamino)ethyl)(hexyl)amino)ethan-1-ol (20-15), 2-((2-(Dinonylamino)ethyl)(nonyl)amino)ethan-1-ol (20-16), 2-((2-(Didodecylamino)ethyl)(nonyl)amino)ethan-1-ol (20-17), 2-((2-(Dinonylamino)ethyl)(dodecyl)amino)ethan-1-ol (20-18), 2-((2-(Didodecylamino)ethyl)amino)ethan-1-ol (20-19), Pentyl6-(dodecyl(2-(dodecyl(2-hydroxyethyl)amino)ethyl)amino)hexanoate (20-2), 2-((2-(Didodecylamino)ethyl)(dodecyl)amino)ethan-1-ol (20-20), 3-((2-(Didodecylamino)ethyl)(dodecyl)amino)propan-1-ol (20-21), 4-((2-(Didodecylamino)ethyl)(dodecyl)amino)butan-1-ol (20-22), (Z)-2-((2-(Didodecylamino)ethyl)(dodec-6-en-1-yl)amino)ethan-1-ol (20-23), 2-((2-(Didodecylamino)ethyl)(tetradecyl)amino)ethan-1-ol (20-24), 2-((2-(Didodecylamino)ethyl)((9Z,12Z)-octadeca-9,12-dien-1-yl)amino)ethan-1-ol (20-25), Pentyl6-((2-(didodecylamino)ethyl)(2-hydroxyethyl)amino)hexanoate (20-3), Dipentyl6,6′-((2-(dodecyl(2-hydroxyethyl)amino)ethyl)azanediyl)dihexanoate (20-4), Diheptyl6,6′-((2-((6-(heptyloxy)-6-oxohexyl)(2hydroxyethyl)amino)ethyl)azanediyl)dihexanoate (20-5), Pentyl6-((2-(dinonylamino)ethyl)(2-hydroxyethyl)amino)hexanoate (20-6), Heptyl6-(dodecyl(2-(dodecyl(2-hydroxyethyl)amino)ethyl)amino)hexanoate (20-7), Nonyl8-((2-(didodecylamino)ethyl)(2-hydroxyethyl)amino)octanoate (20-8), Heptadecan-9-yl8-((2-(didodecylamino)ethyl)(2-hydroxyethyl)amino)octanoate (20-9), 1-(2,2-Di((9Z,12Z)-octadeca-9,12-dien-1-yl)cyclopropyl)-N,N-dimethylmethanamine (21-1), 3,3-Di((9Z,12Z)-octadeca-9,12-dien-1-yl)cyclobutyl4-(dimethylamino)butanoate (21-2), 3,3-Di((9Z,12Z)-octadeca-9,12-dien-1-yl)cyclopentyl3-(dimethylamino)propanoate (21-3), 3,3-Di((9Z,12Z)-octadeca-9,12-dien-1-yl)cyclopentyl4-(dimethylamino)butanoate (21-4), 1-(2,3-Di((8Z,11Z)-heptadeca-8,11-dien-1-yl)cyclopropyl)-N,N-dimethylmethanamine (21-6), poly{4-((2-(dimethylamino)ethyl)thio)tetrahydro-2H-pyran-2-one}-r-poly{4-(octylthio)tetrahydro-2H-pyran-2-one} (A7), (3aR5s,6aS)-N,N-dimethyl-2,2-di((9Z,12Z)-octadeca-9,12-dienyl)tetrahydro-3aH-cyclopentad1,3dioxol-5-amine (ALN100), (3aR,5s,6aS)-N,N-dimethyl-2,2-di((9Z,12Z)-octadeca-9,12-dienyl)tetrahydro-3a-H-cyclopenta[d][1,3]dioxol-5-amine (ALN1001), ((3aR,5s,6aS)-N,N-dimethyl-2,2-di((9Z,12Z)-octadeca-9,12-dienyl)tetrahydro-3aH-cyclopenta[d][1,3]dioxol-5-amine)) (ALNY-100), dimyristoyl trimethylammoniumpropane (Amino Lipid 6), N,N-dihydroxyethylmethyl-N-2-(cholesteryloxycarbonylamino)ethylammoniumbromide (BHEM-Chol), N,N-bis-(2-hydroxyethyl)-N-methyl-N-(2-cholesteryloxycarbonylamino-ethyl)ammoniumbromide (BHEM-Chol), (, 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), Cholesteryl-succinyl Silane (C2), (9Z,9′Z,12Z,12′Z)-2-((4-(((3-(dimethylamino)propoxy)carbonyl)oxy)hexadecanoyl)oxy)propane-1,3-diylbis(octadeca-9,12-dienoate) (Cationic Lipid A2), 9Z,12Z)-3-((4,4-bis(octyloxy)butanoyl)oxy)-2-((((3-(diethylamino)propoxy)carbonyl)oxy)methyl)propyloctadeca-9,12-dienoate (Cationic Lipid A3), 1-(3-cholesteryl)-oxycarbonyl-aminomethylimidazole (CHIM), [(2-Morpholine-4-yl-ethylcarbamoyl)methyl]-carbamicacidcholesterylester (Chol-C3N-Mo2), [(2-Morpholine-4-yl-ethylcarbamoyl)-ethyl]-carbamicacidcholesterylesterChol-DMC3N-Mo2[1-Methyl-2-(2-morpholine-4-yl-ethylcarbamoyl)-propyl]-carbamicacidcholesterylester (Chol-C4N-Mo2), 1,17-bis(2-octylcyclopropyl)heptadecan-9-yl4-(dimethylamino)butanoate (CL), heptatriaconta-6,9,28,31-tetraen-19-yl-4-(dimethylamino)-butanoate (CLO1), cholesteryl3-(dimethylamino)propanoate (CL06), cholesteryl2-(dimethylamino)acetate (CL08), N,N-dimethyl-2,3-bis(((9Z,12Z)-octadeca-9,12-dien-1-yl)oxy)propan-1-amine (CL-1), N-methyl-2-(((9Z,12Z)-octadeca-9,12-dien-1-yl)oxy)-N-(2-((((9Z,12Z)-octadeca-9,12-diene-1-yl)oxy)ethyl)ethan-1-amine (CL-11), (3R,4R)-3,4-bis(((Z)-hexadec-9-en-1-yl)oxy)-1-methylpyrrolidine(CompoundCL-12) (CL-12), 2-(Dimethylamino)-N-((6Z,9Z,28Z,31Z)-Heptatriconta-6,9,28,31-tetraen-19-yl)acetamide (CL-13), 3-(Dimethylamino)propane-1,2-diyl(9Z,9′Z,12Z,12′Z)-bis(octadeca-9,12-dienoate) (CL-14), (9Z,12Z)-di((9Z,12Z)-octadeca-9,12-dien-1-yl)amine (CL-15), 7-Hydroxy7-(4-((1-methylpiperidine-4-carbonyl)oxy)butyl)tridecane-1,13-diyldidodecanoate (CL15B6), 7-Hydroxy7-(4-((1-methylpiperidine-4-carbonyl)oxy)butyl)tridecane-1,13-diylditetradecanoate (CL15C6), 7-Hydroxy7-(4-((1-methylpiperidine-4-carbonyl)oxy)butyl)tridecane-1,13-diyldipalmitate (CL15D6), 7-Hydroxy7-(4-((1-methylpiperidine-4-carbonyl)oxy)butyl)tridecane-1,13-diyldioleate (CL15H6), Bis(2-(((9Z,12Z)-octadeca-9,12-dien-1-yl)oxy)ethyl)amine (CL-16), (9Z,12Z)—N-Methyl-N-(2-(((9Z,12Z)-octadeca-9,12-dien-1-yl)oxy)ethyl)octadeca-9,12-dien-1-amine (CL-17), (9Z,12Z)—N-(3-(((9Z,12Z)-octadeca-9,12-dien-1-yl)oxy)propyl)octadeca-9,12-dien-1-amine (CL-18), (1-Methylpiperidin-3-yl)methyldi((11Z,14Z)-icosa-11,14-dien-1-yl)carbamate (CL-19), N-methyl-N,N-bis(2-((Z)-hexadec-9-enyloxy)ethyl)amine (CL-2), (13Z,16Z)—N,N-Dimethyl-4-((9Z,12Z)-octadeca-9,12-dien-1-yl)docosa-3,13,16-trien-1-amine (CL-20), (S)-2-Amino-3-hydroxy-N,N-bis(2-(((Z)-octadeca-9-en-1-yl)oxy)ethyl)propanamide (CL-21), C2:N,N-dihexadecyl-N′-(3-triethoxysilylpropyl)succinamide (CL3), trans-1-Methyl-3,4-bis((((Z)-octadec-9-en-1-yl)oxy)methyl)pyrrolidine (CL-3), trans-1-methylpyrrolidine-3,4-diyl)bis(methylene)(9Z,9′Z,12Z,12′Z)-bis(octadeca-9,12-dienoate) (CL-4), 7-(4-(Diisopropylamino)butyl)-7-hydroxytridecane-1,13-diylditetradecanoate (CL4C6), 7-(4-(Diisopropylamino)butyl)-7-hydroxytridecane-1,13-diyldipalmitate (CL4D6), 11-(4-(Diisopropylamino)butyl)-11-hydroxyhenicosane-1,21-diyldioleate (CL4H10), 7-(4-(Diisopropylamino)butyl)-7-hydroxytridecane-1,13-diyldioleate (CL4H6), 9-(4-(Diisopropylamino)butyl)-7-hydroxyheptadecane-1,17-diyldioleate (CL4H8), (6Z,9Z,28Z,31Z)-Heptatriaconta-6,9,28,31-tetraen-19-yl4-(dimethylamino)butanoate (CL-5), 2-(Dimethylamino)-N-(2-(((Z)-octadeca-9-en-1-yl)oxy)ethyl)-N-((9Z,12Z)-octadeca-9,12-diene-1-yl)acetamide (CL-53), 3-((2-(((Z)-octadeca-9-en-1-yl)oxy)ethyl)((9Z,12Z)-octadeca-9,12-dien-1-yl)amino)propane-1-All (CL-54), 1-Methyl-3,3-bis((((9Z,12Z)-octadeca-9,12-dien-1-yl)oxy)methyl)azetidine (CL-55), 1-Methyl-3,3-bis(2-(((9Z,12Z)-octadeca-9,12-dien-1-yl)oxy)ethyl)azetidine (CL-56), 1-Methyl-3,3-bis(2-(((9Z,12Z)-octadeca-9,12-dien-1-yl)oxy)propyl)azetidine (CL-57), 2-(3,3-di((9Z,12Z)-octadeca-9,12-dien-1-yl)azetidin-1-yl)ethan-1-ol (CL-58), 2-(3,3-di((9Z,12Z)-octadeca-9,12-dien-1-yl)azetidin-1-yl)propan-1-ol (CL-59), 3-(Di((9Z,12Z)-octadeca-9,12-dien-1-yl)amino)propan-1-o (CL-6), 3-(Dimethylamino)propyl3,3-di((9Z,12Z)-octadeca-9,12-dien-1-yl)azetidine-1-carboxylate (CL-60), 2-(Di((Z)-octadeca-9-en-1-yl)amino)ethane-1-ol (CL-61), 3-(Di((Z)-octadeca-9-en-1-yl)amino)propan-1-ol (CL-62), (11Z,14Z)-2-((Dimethylamino)methyl)-2-((9Z,12Z)-octadeca-9,12-dien-1-yl)icosa-11,14-dien-1-ol (CL-63), (11Z,14Z)-2-(Dimethylamino)-2-((9Z,12Z)-octadeca-9,12-dien-1-yl)icosa-11,14-dien-1-ol (CL-64), 3-(Dimethylamino)-2,2-bis((((9Z,12Z)-octadeca-9,12-dien-1-yl)oxy)methyl)propan-1-ol (CL-65), (9Z,12Z)—N-(2-(((Z)-Octadeca-9-en-1-yl)oxy)ethyl)octadeca-9,12-dien-1-amine (CL-7), 1-Methyl-3,3-di((9Z,12Z)-octadeca-9,12-dien-1-yl)azetidine (CL-8), N,2-Dimethyl-1,3-bis(((9Z,12Z)-octadeca-9,12-dien-1-yl)oxy)propan-2-amine (CL-9), 3-Dimethylamino-2-(Cholest-5-en-3B-oxybutan-4-oxy)-1-(cis,cis-9,12-octadecadienoxy)propane (CLinDMA), 2-[5′-(cholest-5-en-3-oxy)-3′-oxapentoxy)-3-dimethy-1-(cis,cis-9′, 12′-octadecadienoxy)propane (CpLinDMA), cetyltrimethylammoniumbromide (CTAB), O,O′-ditetradecanoyl-N-(a-trimethylammonioacetyl)diethanolaminechloride (DC-6-14), 30-[N—(N′,N′-dimethylaminoethane)carbamoyl]cholesterol (DC-Chol), dimethyldioctadecylammonium (DDA), dimethyldioctadecylammoniumbromide (DDA), N,N-distearyl-N,N-dimethylammoniumbromide (DDAB), N-(2-(dimethylamino)ethyl)-4,5-bis(dodecylthio)pentanamide (DEDPA), 3-Dimethylamino-2-(Cholest-5-en-30-oxypent-3-oxa-an-5-oxy)-1-(cis,cis-9,12-octadecadienoxy)propane (DEG-CLinDMA), 1,6-Dileoyl Triethylenetetramide (dio-TETA), N1,N19-bis((S,23E,25E,27E,29E)-16-((2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide (diVA-PEG-diVA), DiLin-N-Methylpiperazine (DL-033), DiLin-N,N-DimethylGlycine (DL-036), Dioleyl-N,N-DimethylGlycine (DL-048), 3-((1,3-bis(((9Z,12Z)-octadeca-9,12-dienoyl)oxy)propan-2-yl)amino)propanoicacid (DLAPA), 1,2-dilinolenyloxy-3-dimethylaminopropane (DLenDMA), 1-Linoleoyl-2-linoleyloxy-3-dimethylaminopropane (DLin-2-DMAP), 3-(N,N-Dilinoleylamino)-1,2-propanediol (DLinAP), 1,2-N,N′-Dilinoleylcarbamyl-3-dimethylaminopropane (DLincarbDAP), 1,2-Dilinoleoylcarbamyl-3-dimethylaminopropane (DLinCDAP), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleoyl-3-dimethylaminopropane (DLinDAP), 1,2-DiLinoleyloxy-N,N-dimethylaminopropane (DLinDMA), 1,2-dilinoleyloxy-3-dimethylaminopropane (DLinDMA 1), 1,2-Dilinoleyloxo-3-(2-N,N-dimethylamino)ethoxypropane (DLin-EG-DMA), dilinoleoyl-4-aminobutyricacid (DLinFAB), 2,2-dilinoleyl-4-(2-dimethylaminoethyl)-[1,3]-dioxolane (DLin-K-C2-DMA), 2,2-Dilinoleyl-4-dimethylaminomethyl-[1,3]-dioxolane (DLin-K-DMA), 1,2-Dilinoleyoxy-3-morpholinopropane (DLin-MA), (6Z,9Z,28Z,31Z)-heptatriacont-6,9,28,31-tetraene-19-yl 4-(dimethylamino)butanoate (DLin-MC3-DMA), 1,2-Dilinoleyloxy-3-(N-methylpiperazino)propane (DLinMPZ), 1,2-Dilinoleyloxy-3-(N-methylpiperazino)propane (DLin-MPZ), Dilinoleyloxy3-piperidinopropylamine (DLinPip), 1.2Dilinoleyloxy3-(3′-hvdroxypiperidino)-propylamine (DLinPip-30H), 1,2Dilinoleyloxy3-(4′-hvdroxypiperidino)-propylamine (DLinPip-40H), 1,2-Dilinoleyloxy-3-hvdroxypropane (DLinPO), 1,2-Dilinoleylthio-3-dimethylaminopropane (DLin-S-DMA), 1,2-Dilinoleoyl-3-trimethylaminopropane (DLinTAP), 1,2-Dilinoleoyl-3-trimethylaminopropanechloridesalt (DLin-TAP.C1), 1,2-Dilinoleyloxy-3-trimethylaminopropane (DLinTMA), 1,2-Dilinoleyloxy-3-trimethylaminopropanechloridesalt (DLin-TMA.C1), 3-((1,3-bis(((9Z,12Z.15Z)-octadeca-9,12,15-trienoyl)oxy)propan-2-yl)amino)propanoic acid (DLLAPA), 1,2Dilinoleyloxy3-(N,Ndimethyl)-propylamine (DLmDEA), 1,2-Dilauroyl-sn-Glicero-3-Phosphoethanolamine (DLPE), 1,2-Dilauroyl-sn-Glicero-3-Glycerol (DLPG), N,N-Dimethyl-3,4-dioleyloxybenzylamine (DMOBA), dimyristoylphosphatidylserine (DMPS), N-[1-(2,3-dimyristyloxy)propyl]-N,N-dimethyl-N-(2-hydroxyethyl)ammoniumbromide (DMRIE), 1,2-Dimyristyloxypropyl-3-dimethyl-hydroxyethylammoniumbromide (DMRIE1), 1,2-dimyristoyl-3-trimethylammoniumpropane (DMTAP), 3-(N,N-Dioleylamino)-1,2-propanedio (DOAP), 3-((1,3-bis(oleoyloxy)propan-2-yl)amino)propanoicacid (DOAPA), 1,2-N,N′-dioleylcarbamyl-3-dimethylaminopropane (DOcarbDAP), 1,2-Dioleoylcarbamyl-3-Dimethylammonium-propane (DOCDAP), N,N-dioleyl-N,N-dimethylammoniumchloride (DODAC), 1,2-Dioleoyl-3-Dimethylammonium-propane (DODAP), N,N-dihydroxyethylN,N-dioctadecylammoniumchloride (DODEAC), N,N-dimethyl-2,3-dioleyloxypropylamine (DODMA), dioleoyl-4-aminobutyric acid (DOFAB), Dioctadecylamidoglycylspermine (DOGS), 1,2-Dioleoyl-3-methyl-(methoxycarbonyl-ethyl)ammonium-Propane (DOMCAP), 1,2-Dioleoyl-3-N-pyrrolidine-propane (DOP5P), 1,2-Dioleoyl-3-N-pyrridinium-propane, bromide salt (DOP6P), 1,2-dioleoyl-3-dimethyl-hydroxyethylammoniumbromide (DORI), 1,2-dioleyloxypropyl-3-dimethyl-hydroxyethylammoniumbromide (DORIE), 1,2-dioleyloxypropyl-3-dimethyl-hydroxybutylammoniumbromide (DORIE-HB), 1,2-dioleyloxypropyl-3-dimetyl-hydroxypropylammoniumbromide (DORIE-HP), 1,2-dioleyloxypropyl-3-dimethyl-hydroxypentylammoniumbromide (DORIE-Hpe), 2,3-dioleyloxy-N-[2(spermine-carboxamido)ethyl]-N,N-dimethyl-1-propanaminiumtrifluoroacetate (DOSPA), 1,3-dioleoyloxy-2-(6-carboxy-spermyl)-propylamide (DOSPER), N-(1-(2,3-dioleoyloxy)propyl)-N,N,N-trimethylammoniumchloride (DOTAP), 1,2-dioleoyl-3-trimethylammonium-propane (DOTAP1), N-[5′-(2′, 3′-dioleoyl)uridine]-N′,N′,N′-trimethylammoniumtosylate (DOTAU), 1-[2-(9(Z)-octadecenoyloxy)ethyl]-2-(8(Z)-heptadecenyl-3-(2-hydroxyethyl)imidazoliniumchloride (DOTIM), N-(1-(2,3-dioleyloxy)propyl)-N,N,N-trimethylammonium chloride (DOTMA), dioleylphosphatidyluridinephosphatidylcholine (DOUPC), 2,3-bis(dodecylthio)propyl(2-(dimethylamino)ethyl)carbamate (DPDEC), dipalmitoyl-4-aminobutyricacid (DPFAB), 1,2-dipalmityloxypropyl-3-dimethyl-hydroxyethylammoniumbromide (DPRIE), 1,2-dipalmitoyl-3-trimethylammoniumpropane (DPTAP), 1-[2-(hexadecanoyloxy)ethyl]-2-pentadecyl-3-(2-hydroxyethyl)imidazoliniumchloride (DPTIM), 3-((1,3-bis(stearoyloxy)propan-2-yl)amino)propanoic acid (DSAPA), distearyldimethylammonium (DSDMA), 1,2-distearloxy-N,N-dimethylaminopropane (DSDMA1), 1,2-disteryloxypropyl-3-dimethyl-hydroxyethylammonium bromide (DSRIE), 1,2-disteroyl-3-trimethylammoniumpropane (DSTAP), ditetradecyltrimethylammonium (DTDTMA), 1,2-dioleoyl-sn-glycero-3-ethylphosphocholine (EDOPC), N2-[N2,N5-bis(3-aminopropyl)-L-ornithyl]-N,N-dioctadecyl-L-glutamine tetrahydrotrifluoroacetate (GC33), Cholest-5-en-3-ol(3P)—,3-[(3-aminopropyl)[4-[(3-aminopropyl)amino]butyl]carbamate] (GL67), glyceryl mono-oleate (GMO), Guanidino-dialkyl-carboxylic acid (GUADACA), 2-(bis(2-(tetradecanoyloxy)ethyl)amino)-N-(2-hydroxyethyl)-N,N-dimethyl-2-oxoethan-aminiumbromide (HEDC), 2,2′-(tert-butoxycarbonylazanediyl)bis(ethane-2,1-diyl)ditetradecanoate (HEDC-BOC-TN), 1-(2-(((3S,10R,13R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yldisulfanyl)ethyl)guanidine (HGT4002), (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15,18-trien-1-amine (HGT5001), Histaminyl-Cholesterol hemisuccinat (HisChol), histidinylcholesterol hemisuccinate (Hist-Chol), HydroSoyPC (HSPC), imidazole cholesterolester (ICE), 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), N,N-di-n-octadecyl,N-mcthyl-N-(2-guanidinyl)cthylammoniumchloride (Lipid 2), 3-((4,4-bis(octyloxy)butanoyl)oxy)-2-((((3-(diethylamino)propoxy)carbonyl)oxy)methyl)propyl(9Z,12Z)-octadeca-9,12-dienoate (Lipid A), (9Z,12Z)-3-((4,4-bis(octyloxy)butanoyl)oxy)-2-((((3-(diethylamino)propoxy)carbonyl)oxy)methyl)propyloctadeca-9,12-dienoate (Lipid A1), 2,2-Dilinoleyl-4-dimethylaminoethyl-[1,3]-dioxolane (Lipid A2), ((5-((dimethylamino)methyl)-1,3-phenylene)bis(oxy))bis(octane-8,1-diyl)bis(decanoate) (Lipid B), 2-((4-(((3-(dimethylamino)propoxy)carbonyl)oxy)hexadecanoyl)oxy)propane-1,3-diyl 9Z,9′Z,12Z,12′Z)-bis(octadeca-9,12-dienoate) (Lipid C), 3-(((3-(dimethylamino)propoxy)carbonyl)oxy)-13-(octanoyloxy)tridecyl 3-octylundecanoate (Lipid D), (6Z,16Z)-12-((Z)-dec-4-en-1-yl)docosa-6,16-dien-11-yl5-(dimethylamino)pentanoate (Lipid I), Dioctadecyl-(2-hydroxyl-3-propylamino)aminopolylysine (Lipid T), (3-((6Z,9Z,28Z,31Z)-heptatriaconta-6,9,28,31-tetraen-19-yloxy)-N,N-dimethylpropan-1-amine (MC3 Ether), described in U.S. Provisional application No. 61/384,050 (MC3 Thioester), (4-((6Z,9Z,28Z,31Z)-heptatriaconta-6,9,28,31-tetraen-19-yloxy)-N,N-dimethylbutan-1-amine (MC4 Ether), 3-((2-(((9Z,12Z)-octadeca-9,12-dienoyl)oxy)ethyl)amino)propanoic acid (MLAPA), 3-((2-(((9Z,12Z,15Z)-octadeca-9,12,15-trienoyl)oxy)ethvnamino)propanoicacid (MLLAPA), monomycolylglycerol (MMG), 3-((2-(oleoyloxy)ethyl)amino)propanoic acid (MOAPA), 4-(2-Aminoethyl)-Morpholino-Cholesterolhemisuccinat (MoChol), 1,2-Dioleoyl-3-N-morpholine-propane (MoDO), Methylpyridiyl-dialkyl-carboxylic acid (MPDACA), monopalmitoylphosphatidylcholine (MPPC), 3-((2-(stearoyloxy)ethyl)amino)propanoic acid (MSAPA), N1-[2-((1S)-1-[(3-aminopropyl)amino]-4-[di(3-amino-propyl)amino]butylcarboxamido)ethyl]-3,4-di[oleyloxy]-benzamide (MVL5), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), phosphatidylcholines (PC), 1,3-Bis-(1,2-bis-tetradecyloxy-propyl-3-dimemylethoxyammonium bromide)-propane-2-ol (PCL-2), palmitoyl-oleoyl-nor-arginine (PONA), stearylamine (STA), 2-(((tert-Butyldimethylsilyl)oxy)methyl)-2-(hydroxymethyl)propane-1,3-diol, 3-((tert-Butyl(dimethyl)silyl)oxy)-2,2-bis(((9Z)-tetradec-9-enoyloxy)methyl)propyl(9Z)-tetradec-9-enoate, 3-Hydroxy-2,2-bis(((9Z)-tetradec-9-enoyloxy)methyl)propyl(9Z)-tetradec-9-enoate, 3-((4-(Dimethylamino)butanoyl)oxy)-2,2-bis(((9Z)-tetradec-9-enoyloxy)methyl)propyl(9Z)-tetradec-9-enoate, 3-(5-(bis(2-hydroxydodecyl)amino)pentan-2-yl)-6-(5-((2-hydroxydodecyl)(2-hydroxyundecyl)amino)pentan-2-yl)-1,4-dioxane-2,5-dione), trehalose-6′, 6′-dibehenate (TDB), 1,1′-(2-(4-(2-((2-(bis(2hydroxydodecyl)amino)ethyl)(2-hydroxydodecyl)amino)ethyl)piperazin-1-yl)ethylazanediyl)didodecan-2-ol (Tech G1), 3-((1,3-bis(((9Z,12Z)-octadeca-9,12-dienoyl)oxy)-2-((((9Z,12Z)-octadeca-9,12-dienoyl)oxy)methyl)propan-2-yl)amino)propanoic acid (TLAPA), 3-((1,3-bis(((9Z.12Z.15Z)-octadeca-9.12.15-trienoyl)oxy)-2-((((9Z.12Z.15E)-octadeca-9,12,15-trienoyl)oxy)methyl)propan-2-yl)amino)propanoicacid (TLLAPA), N-(a-trimethylammonioacetyl)-didodecyl-D-glutamate chloride (TMAG), 3-((1,3-bis(((Z)-octadec-9-enoyl)oxy)-2-((((Z)-octadec-9-enoyl)oxy)methyl)propan-2-yl)amino)propanoic acid (TOAPA), 3-((1,3-bis(stearoyloxy)-2-((stearoyloxy)methyl)propan-2-yl)amino)propanoic acid (TSAPA), 1,N19-bis((16E,18E,20E,22E)-17,21-dimethyl-15-oxo-23-(2,6,6-trimethylcyclohex-1-en-1-yl)-4,7,10-trioxa-14-azatricosa-16,18,20,22-tetraen-1-yl)-4,7,10,13,16-pentaoxanonadecane-1,19-diamide (VA-PEG-VA), 2,2-Dillinoleyl-4-dimethylaminoethyl-[1,3]-dioxolane (XTC), disclosed in Non-Patent Literaturel 1 (YSK05), 1,2-di-7-linolenyloxy-N,N-dimethylaminopropane (y-DLenDMA), a-D-Tocopherol hemisuccinoyl, (9Z,9,Z,12Z,12,Z)-2-((2-(((3-(dimethylamino)propoxy)carbonyl)oxy)tetradecanoyl)oxy)propane-1,3-diylbis(octadeca-9,12-dienoate), 2-(((13Z,16Z)-4-(((3-(diethylamino)propoxy)carbonyl)oxy)docosa-13,16-dienoyl)oxy)propane-1,3-diyldioctanoate, 2-(((13Z,16Z)-4-(((3-(dimethylamino)propoxy)carbonyl)oxy)docosa-13,16-dienoyl)oxy)propane-1,3-diyldioctanoate, 2-((4-(((3-(ethyl(methyl)amino)propoxy)carbonyl)oxy)hexadecanoyl)oxy)propane-1,3-diyldioctanoate, 2-((4-(((3-(ethyl(methyl)amino)propoxy)carbonyl)oxy)hexadecanoyl)oxy)propane-1,3-diylbis(decanoate), 2-((4-(((3-(diethylamino)propoxy)carbonyl)oxy)hexadecanoyl)oxy)propane-1,3-diylbis(decanoate), 2-(10-dodecyl-3-ethyl-8,14-dioxo-7,9,13-trioxa-3-azaicosan-20-yl)propane-1,3-diyldioctanoate, 2-(((4-(dimethylamino)butanoyl)oxy)methyl)-2-((octanoyloxy)methyl)propane-1,3-diyl(9Z,9′Z)bis-tetradec-9-enoate, (9Z,9′Z,12Z,12′Z)-2-(((1-(cyclopropylmethyl)piperidine-4-carbonyl)oxy)methyl)propane-1,3-diylbis(octadeca-9,12-dienoate), ((2-(((1-isopropylpiperidine-4-carbonyl)oxy)methyl)-1,4-phenylene)bis(oxy))bis(octane-8,1-diyl)bis(decanoate), 2-((4-(((3-(ethyl(methyl)amino)propoxy)carbonyl)oxy)hexadecanoyl)oxy)propane-1,3-diyldidodecanoate, 2-((4-(((3-(diethylamino)propoxy)carbonyl)oxy)hexadecanoyl)oxy)propane-1,3-diyldidodecanoate, 2-((4-(((3-(dimethylamino)propoxy)carbonyl)oxy)hexadecanoyl)oxy)propane-1,3-diyldidodecanoate, 2-((4-(((3-(ethyl(methyl)amino)propoxy)carbonyl)oxy)hexadecanoyl)oxy)propane-1,3-diylditetradecanoate, 2-((4-(((3-(dimethylamino)propoxy)carbonyl)oxy)hexadecanoyl)oxy)propane-1,3-diylditetradecanoate, 2-((4-(((3-(diethylamino)propoxy)carbonyl)oxy)hexadecanoyl)oxy)propane-1,3-diylditetradecanoate, (Z)-2-((4-(((3-(dimethylamino)propoxy)carbonyl)oxy)hexadecanoyl)oxy)propane-1,3-diyldioleate, (9Z,9,Z,12Z,12,Z,15Z,15,Z)-2-((4-(((3-(dimethylamino)propoxy)carbonyl)oxy)hexadecanoyl)oxy)propane-1,3-diylbis(octadeca-9,12,15-trienoate), (9Z,9,Z,12Z,12,Z)-2-((4-(((3-(diethylamino)propoxy)carbonyl)oxy)hexadecanoyl)oxy)propane-1,3-diylbis(octadeca-9,12-dienoate), (9Z,9,Z,12Z,12,Z)-2-((4-(((3-(dimethylamino)propoxy)carbonyl)oxy)hexadecanoyl)oxy)propane-1,3-diylbis(octadeca-9,12-dienoate), N,N,N-trimethyl-5-oxo-5-(3-((3-pentyloctanoyl)oxy)-2,2-bis(((3-pentyloctanoyl)oxy)methyl)propoxy)pentane-1-Aminiumiodide3-((5-(dimethylamino)pentanoyl)oxy)-2,2-bis(((3-pentyloctanoyl)oxy)methyl)propyl3-pentyloctanoate, 3-dimethylaminopropylcarbonate(9Z,12Z)-octacosa-19,22-dien-11-yl, 2-(((N,N-dimethyl-p-alanyl)oxy]methyl}-2-[(octanoyloxy)methyl)propane-1,3-diyl(9Z,9′Z)bis-tetradec-9-enoate, 0′1,01-(2-(7-dodecyl-14-methyl-3,9-dioxo-2,4,8,10-tetraoxa-14-azapentadecyl)propane-1,3-diyl)8-dimethyldioctanedioate, 8-dimethylO′I,O1-(2-(((1-methylpyrrolidine-3-carbonyl)oxy)methyl)propane-1,3-diyl)dioctanedioate, 1-(3-((6,6-bis((2-propylpentyl)oxy)hexanoyl)oxy)-2-(((1,4-dimethylpiperidine-4-carbonyl)oxy)methyl)propyl)8-methyloctanedioate, (9Z,12Z)-5-(((3-(dimethylamino)propoxy)carbonyl)oxy)-7-octylpentadecyloctadeca-9,12-dienoate, 5-(((3-(dimethylamino)propoxy)carbonyl)oxy)-7-octylpentadecyloctanoate, 1-(3-((6,6-bis((2-propylpentyl)oxy)hexanoyl)oxy)-2-(((1,4-dimethylpiperidine-4-carbonyl)oxy)methyl)propyl)10-octyldecanedioate, 3-(((3-(dimethylamino)propoxy)carbonyl)oxy)-5-octyltridecyldecanoate, 1-(16-(((4,4-bis(octyloxy)butanoyl)oxy)methyl)-9-dodecyl-2-methyl-7,13-dioxo-6,8,12,14-tetraoxa-2-azaheptadecan-17-yl)8-methyloctanedioate, 3-((5-(dimethylamino)pentanoyl)oxy)-2,2-bis(((9Z)-tetradec-9-enoyloxy)methyl)propyl(9Z,12Z)-octadec-9,12-dienoate, 3-((5-(Dimethylamino)pentanoyl)oxy)-2,2-bis(((3-pentyloctanoyl)oxy)methyl)propyl3-pentyloctanoate, (9Z,9′Z,12Z,12′Z)-2-(((3-(diethylamino)propanoyl)oxy)methyl)propane-1,3-diylbis(octadeca-9,12-dienoate), ((2-(((4-(dimethylamino)butanoyl)oxy)methyl)-1,4-phenylene)bis(oxy))bis(octane-8,1-diyl)bis(decanoate), 1-(3-((4,4-bis(octyloxy)butanoyl)oxy)-2-(((1-methylpyrrolidine-3-carbonyl)oxy)methyl)propyl)8-methyloctanedioate, 3-((4,4-bis(octyloxy)butanoyl)oxy)-2-((palmitoyloxy)methyl)propyll-methylpyrrolidine-3-carboxylate, 3-((4,4-bis(octyloxy)butanoyl)oxy)-2-((tetradecanoyloxy)methyl)propyll-methylpyrrolidine-3-carboxylate, 3-(((3-(dimethylamino)propoxy)carbonyl)oxy)-13-(octanoyloxy)tridecyl9-pentyltetradecanoate, 3-((4,4-bis(octyloxy)butanoyl)oxy)-2-((dodecanoyloxy)methyl)propyll-methylpyrrolidine-3-carboxylate, 3-(((3-(dimethylamino)propoxy)carbonyl)oxy)-13-hydroxytridecyl9-pentyltetradecanoate, 3-(((3-(dimethylamino)propoxy)carbonyl)oxy)-13-(octanoyloxy)tridecyl7-hexyltridecanoate, 2-(5-(3-((1-methylpyrrolidine-3-carbonyl)oxy)-2-((tetradecanoyloxy)methyl)propoxy)-5-oxopentyl)propane-1,3-diyldioctanoate, 3-(((3-(dimethylamino)propoxy)carbonyl)oxy)-13-(octanoyloxy)tridecyl5-heptyldodecanoate, 2-(5-(3-((1-methylpyrrolidine-3-carbonyl)oxy)-2-((palmitoyloxy)methyl)propoxy)-5-oxopentyl)propane-1,3-diyldioctanoate, 3-(((3-(dimethylamino)propoxy)carbonyl)oxy)-13-hydroxytridecyl5-heptyldodecanoate, 2-(((1-methylpyrrolidine-3-carbonyl)oxy)methyl)propane-1,3-diylbis(6,6-bis(octyloxy)hexanoate), (9Z,12Z)-3-(((3-dimethylamino)propoxy)carbonyl)oxy)-13-(octanoyloxy)tridecyloctadeca-9,12-dienoate, 3-((5-(dimethylamino)pentanoyl)oxy)-2,2-bis(((9Z)-tetradec-9-enoyloxy)methyl)propyl(9Z)-octadec-9-enoate, 2-(10-dodecyl-3-ethyl-8,14-dioxo-7,9,13-trioxa-3-azanonadecan-19-yl)propane-1,3-diyldioctanoate, ((2-(((1-methylpiperidine-4-carbonyl)oxy)methyl)-1,4-phenylene)bis(oxy))bis(octane-8,1-diyl)bis(decanoate), 2-(((3-(dimethylamino)propanoyl)oxy)methyl)propane-1,3-diylbis(4,4-bis(octyloxy)butanoate), (9Z,12Z)-2-(((11Z,14Z)-2-((3-(dimethylamino)propanoyl)oxy)icosa-11,14-dien-1-yl)oxy)ethyloctadeca-9,12-dienoate, 2-(((1,3-dimethylpyrrolidine-3-carbonyl)oxy)methyl)propane-1,3-diylbis(4,4-bis(octyloxy)butanoate), (13Z,16Z)-4-(((3-(dimethylamino)propoxy)carbonyl)oxy)docosa-13,16-dien-1-ylheptadecan-9-ylsuccinate, 2,2-bis(heptyloxy)ethyl3-((3-ethyl-10-((9Z,12Z)-octadeca-9,12-dien-1-yl)-8,15-dioxo-7,9,14-trioxa-3-azaheptadecan-17-yl)disulfanyl)propanoate, 2-(((1-methylpyrrolidine-3-carbonyl)oxy)methyl)propane-1,3-diylbis(4,4-bis(octyloxy)buta, 1-(3-((1,3-dimethylpyrrolidine-3-carbonyl)oxy)-2-(((9Z,12Z)-octadeca-9,12-dienoyloxy)methyl)propyl)10-octyldecanedioate, (13Z,16Z)-4-(((3-(diethylamino)propoxy)carbonyl)oxy)docosa-13,16-dien-1-yl2,2-bis(heptyloxy)acetate, (13Z,16Z)-4-(((2-(dimethylamino)ethoxy)carbonyl)oxy)docosa-13,16-dien-1-yl2,2-bis(heptyloxy)acetate, Aceticacid(20,23R)-2-methyl-9-[(9Z,12Z)-octadeca-9,12-dien-1-yl]-7-oxo-6,8,11-trioxa-2-azanonacosa-20-En-23-yl3-(dimethylamino)propylcarbonate(11Z,14Z)-1-{[(9Z,12R)-12-hydroxyoctadec-9-en-1-yl], (12Z,15Z)-1-((((9Z,12Z)-octadeca-9,12-dien-1-yloxy)carbonyl)oxy)henicosa-12,15-dien-3-yl3-(dimethylamino)propanoate, (9Z,12Z)-3-((4,4-bis(octyloxy)butanoyl)oxy)-2-((((3-(dimethylamino)propyl)carbamoyl)oxy)methyl)propyloctadeca-9,12-dienoate, (12Z,15Z)-3-((4-(dimethylamino)butanoyl)oxy)henicosa-12,15-dien-1-yl9-pentyltetradecanoate, (9Z,12Z)-3-((4,4-bis(octyloxy)butanoyl)oxy)-2-(((((1,2,2,6,6-pentamethylpiperidin-4-yl)oxy)carbonyl)oxy)methyl)propyloctadeca-9,12-dienoate, (12Z,15Z)-3-((4-(dimethylamino)butanoyl)oxy)henicosa-12,15-dien-1-yl7-hexyltridecanoate, (9Z,12Z)-3-((4,4-bis(octyloxy)butanoyl)oxy)-2-(((((1-methylpiperidin-4-yl)methoxy)carbonyl)oxy)methyl)propyloctadeca-9,12-dienoate, (12Z,15Z)-3-((4-(dimethylamino)butanoyl)oxy)henicosa-12,15-dien-1-yl5-heptyldodecanoate, (9Z,12Z)-3-((4,4-bis(octyloxy)butanoyl)oxy)-2-(((((1-ethylpiperidin-4-yl)oxy)carbonyl)oxy)methyl)propyloctadeca-9,12-dienoate, (12Z,15Z)-3-((4-(dimethylamino)butanoyl)oxy)henicosa-12,15-dien-1-yl3-octylundecanoate,formatesalt, 3-((5-(dimethylamino)pentanoyl)oxy)-2,2-bis(((9Z)-tetradec-9-enoyloxy)methyl)propyl(9Z)-hexadec-9-enoate, (9Z,12Z)-3-((4,4-bis(octyloxy)butanoyl)oxy)-2-(((((1-methylazetidin-3-yl)oxy)carbonyl)oxy)methyl)propyloctadeca-9,12-dienoate, (9Z,12Z)-(12Z,15Z)-3-((3-(dimethylamino)propanoyl)oxy)henicosa-12,15-dien-1-yloctadeca-9,12-dienoate, 2-(((3-(diethylamino)propoxy)carbonyl)oxy)tetradecyl4,4-bis((2-ethylhexyl)oxy)butanoate, (9Z,12Z)-3-((4,4-bis(octyloxy)butanoyl)oxy)-2-(((((1-methylpiperidin-4-yl)oxy)carbonyl)oxy)methyl)propyloctadeca-9,12-dienoate, (9Z,12Z)-3-((4,4-bis(octyloxy)butanoyl)oxy)-2-(((((1-methylpyrrolidin-3-yl)oxy)carbonyl)oxy)methyl)propyloctadeca-9,12-dienoate, (9Z,12Z)-3-(((2-(dimethylamino)ethoxy)carbonyl)oxy)pentadecyloctadeca-9,12-dienoate, (9Z,12Z)-3-((4,4-bis(octyloxy)butanoyl)oxy)-2-((((3-(4-methylpiperazin-1-yl)propoxy)carbonyl)oxy)methyl)propyloctadeca-9,12-dienoate, 3-(Dimethylamino)propyltriacontan-11-ylcarbonateTriacontan-11-ol, (9Z,12Z)-3-((4,4-bis(octyloxy)butanoyl)oxy)-2-((((3-(pyrrolidin-1-yl)propoxy)carbonyl)oxy)methyl)propyloctadeca-9,12-dienoate, (9Z,12Z)-3-(((3-(ethyl(methyl)amino)propoxy)carbonyl)oxy)pentadecyloctadeca-9,12-dienoate, 3-((4,4-bis(octyloxy)butanoyl)oxy)-2-(((9Z,12Z)-octadeca-9,12-dienoyloxy)methyl)propyl4-((diethylamino)methyl)benzoate, (9Z,12Z)-3-(((3-(diethylamino)propoxy)carbonyl)oxy)pentadecyloctadeca-9,12-dienoate, 3-((4,4-bis(octyloxy)butanoyl)oxy)-2-(((9Z,12Z)-octadeca-9,12-dienoyloxy)methyl)propyl3-((dimethylamino)methyl)benzoate, (9Z,12Z)-3-(((3-(dimethylamino)propoxy)carbonyl)oxy)pentadecyloctadeca-9,12-dienoate, 3-((4,4-bis(octyloxy)butanoyl)oxy)-2-(((9Z,12Z)-octadeca-9,12-dienoyloxy)methyl)propyll-methylpiperidine-3-carboxylate, 3-((4,4-bis(octyloxy)butanoyl)oxy)-2-(((9Z,12Z)-octadeca-9,12-dienoyloxy)methyl)propyll-methylpiperidine-4-carboxylate, 3-((4,4-bis(octyloxy)butanoyl)oxy)-2-(((9Z,12Z)-octadeca-9,12-dienoyloxy)methyl)propyl1,4-dimethylpiperidine-4-carboxylate, 3-((4-(dimethylamino)butanoyl)oxy)-2,2-bis(((9Z)-tetradec-9-enoyloxy)methyl)propyl(9Z)-hexadec-9-enoate, 2-(10-dodecyl-3-ethyl-8,14-dioxo-7,9,13-trioxa-3-azahexadecan-16-yl)propane-1,3-diyldioctanoate, (9Z,9′Z,12Z,12′Z)-2-(((4-(piperidin-1-yl)butanoyl)oxy)methyl)propane-1,3-diylbis(octadeca-9,12-dienoate), 3-((4,4-bis(octyloxy)butanoyl)oxy)-2-(((9Z,12Z)-octadeca-9,12-dienoyloxy)methyl)propyl4-methylmorpholine-2-carboxylate, (2R)-3-((4,4-bis(octyloxy)butanoyl)oxy)-2-(((9Z,12Z)-octadeca-9,12-dienoyloxy)methyl)propyll-methylpyrrolidine-2-carboxylate, (2S)-3-((4,4-bis(octyloxy)butanoyl)oxy)-2-(((9Z,12Z)-octadeca-9,12-dienoyloxy)methyl)propyll-methylpyrrolidine-2-carboxylate, (9Z,9′Z,12Z,12′Z)-2-((((3-(diethylamino)propoxy)carbonyl)oxy)methyl)-2-(((9Z,12Z)-octadeca-9,12-dienoyloxy)methyl)propane-1,3-diylbis(octadeca-9,12-dienoate), (9Z,12Z)-3-((4,4-bis(octyloxy)butanoyl)oxy)-2-(((((1-ethylpiperidin-3-yl)methoxy)carbonyl)oxy)methyl)propyloctadeca-9,12-dienoate, 3-((4,4-bis(octyloxy)butanoyl)oxy)-2-(((9Z,12Z)-octadeca-9,12-dienoyloxy)methyl)propyll-(cyclopropylmethyl)piperidine-4-carboxylate, 3-((4,4-bis(octyloxy)butanoyl)oxy)-2-(((9Z,12Z)-octadeca-9,12-dienoyloxy)methyl)propyll-isopropylpiperidine-4-carboxylate, (9Z,12Z)-3-((4,4-bis(octyloxy)butanoyl)oxy)-2-(((3-(dimethylamino)propanoyl)oxy)methyl)propyloctadeca-9,12-dienoate, 4-(dimethylamino)butylcarbonate(6Z,9Z,26Z,29Z)-pentatriaconta-6,9,26,29-tetraen-18-yl, 3-((6-(dimethylamino)hexanoyl)oxy)-2,2-bis(((9Z)-tetradec-9-enoyloxy)methyl)propyl(9Z)-tetradec-9-enoate, 2,5-bis((9Z,12Z)-octadeca-9,12-dienyloxy)benzyl3-(dimethylamino)propylcarbonate, (9Z,9′Z,12Z,12′Z)-2-(((4-(pyrrolidin-1-yl)butanoyl)oxy)methyl)propane-1,3-diylbis(octadeca-9,12-dienoate), 3-(((3-(dimethylamino)propoxy)carbonyl)oxy)pentadecyl5-heptyldodecanoate, Aceticacid(7R,9Z)-18-({[3-(dimethylamino)propyloxy]carbonyl}oxy)octacosa-9-en-7-yl, 3-(((3-(dimethylamino)propoxy)carbonyl)oxy)pentadecyl9-pentyltetradecanoate, (9Z,12Z)-3-((6,6-bis(octyloxy)hexanoyl)oxy)-2-((((3-(diethylamino)propoxy)carbonyl)oxy)methyl)propyloctadeca-9,12-dienoate, 3-(((3-(dimethylamino)propoxy)carbonyl)oxy)pentadecyl7-hexyltridec-6-enoate, (9Z,12Z)-3-(2,2-bis(heptyloxy)acetoxy)-2-((((2-(dimethylamino)ethoxy)carbonyl)oxy)methyl)propyloctadeca-9,12-dienoate, 3-(((3-(dimethylamino)propoxy)carbonyl)oxy)pentadecyl3-octylundec-2-enoate, (9Z,12Z)-3-(((3-(diethylamino)propoxy)carbonyl)oxy)-2-(((5-heptyldodecanoyl)oxy)methyl)propyloctadeca-9,12-dienoate, 3-(((3-dimethylamino)propoxy)carbonyl)oxy)pentadecyl3octylundecanoate, (9Z,12Z)-3-(((3-(diethylamino)propoxy)carbonyl)oxy)-2-(((9-pentyltetradecanoyl)oxy)methyl)propyloctadeca-9,12-dienoate, Diaceticacid(7R,9Z,26Z,29R)-18-({[3-(dimethylamino)propoxy]carbonyl}oxy)pentatriaconta-9,26-diene-7,29-diyl, 3-(((3-(dimethylamino)propoxy)carbonyl)oxy)pentadecyl8,8-bis((2-propylpentyl)oxy)octanoate, (9Z,12Z)-3-(((3-(diethylamino)propoxy)carbonyl)oxy)-2-(((7-hexyltridecanoyl)oxy)methyl)propyloctadeca-9,12-dienoate, 3-(((3-(ethyl(methyl)amino)propoxy)carbonyl)oxy)pentadecyl8,8-bis((2-propylpentyl)oxy)octanoate, (9Z,12Z)-3-(((3-(diethylamino)propoxy)carbonyl)oxy)-2-(((3-octylundecanoyl)oxy)methyl)propyloctadeca-9,12-dienoate, 3-(((3-(diethylamino)propoxy)carbonyl)oxy)pentadecyl8,8-bis((2-propylpentyl)oxy)octanoate, 3-(((3-(diethylamino)propoxy)carbonyl)oxy)pentadecyl8,8-dibutoxyoctanoate, 3-((5-(dimethylamino)pentanoyl)oxy)-2,2-bis(((9Z)-tetradec-9-enoyloxy)methyl)propyl(9Z)-tetradec-9-enoate, 3-(Dimethylamino)propylcarbonate(6Z,9Z,26Z,29Z)-pentatriacontour-6,9,26,29-tetraen-18-yl, 2,5-bis((9Z,12Z)-octadeca-9,12-dien-1-yloxy)benzyl3-(dimethylamino)propanoate, (9Z,9′Z,12Z,12′Z)-2-(((3-(4-methylpiperazin-1-yl)propanoyl)oxy)methyl)propane-1,3-diylbis(octadeca-9,12-dienoate), 3-(((3-(diethylamino)propoxy)carbonyl)oxy)pentadecyl8,8-bis(octyloxy)octanoate, 3-(dimethylamino)propyloctacosane-1l-yl carbonate, 2,4-bis((9Z,12Z)-octadeca-9,12-dienyloxy)benzyl4-(dimethylamino)butanoate, (9Z,12Z)-3-(((3-(diethylamino)propoxy)carbonyl)oxy)-2-(((2-heptylundecanoyl)oxy)methyl)propyloctadeca-9,12-dienoate, 3-(((3-(diethylamino)propoxy)carbonyl)oxy)pentadecyl6,6-bis((2-ethylhexyl)oxy)hexanoate, 2-((((3-(dimethylamino)propoxy)carbonyl)oxy)methyl)propane-1,3-diylbis(2-heptylundecanoate), 3-(((3-(diethylamino)propoxy)carbonyl)oxy)pentadecyl6,6-bis(hexyloxy)hexanoate, 4-methyl-2,5-bis((9Z,12Z)-octadeca-9,12-dien-1-yloxy)benzyl4-(dimethylamino)butanoate, 3-(((3-(diethylamino)propoxy)carbonyl)oxy)pentadecyl6,6-bis(octyloxy)hexanoate, 4-(dimethylamino)butyl4-methyl-2,5-bis((9Z,12Z)-octadeca-9,12-dienyloxy)benzylcarbonate, 3-(((3-(dimethylamino)propoxy)carbonyl)oxy)pendadecyl4,4-bis((2-propylpentyl)oxy)butanoate, 2-(12-dodecyl-3-ethyl-8,14-dioxo-7,9,13-trioxa-3-azaoctadecan-18-yl)propane-1,3-diyldioctanoate, 2-(5-oxo-5-((3-(((3-(piperidin-1-yl)propoxy)carbonyl)oxy)pentadecyl)oxy)pentyl)propane-1,3-diyldioctanoate, 3-(dimethylamino)propyl4-methyl-2,5-bis((9Z,12Z)-octadeca-9,12-dien-1-yloxy)benzylcarbonate, 3-(((3-(ethyl(methyl)amino)propoxy)carbonyl)oxy)pentadecyl4,4-bis((2-propylpentyl)oxy)butanoate, 2-(11-dodecyl-3-ethyl-9,15-dioxo-8,10,14-trioxa-3-azanonadecan-19-yl)propane-1,3-diyldioctanoate, 2-(10-dodecyl-3-ethyl-8,15-dioxo-7,9,14-trioxa-3-azanonadecan-19-yl)propane-1,3-diyldioctanoate, 2-(5-((4-((((1-methylpiperidin-4-yl)oxy)carbonyl)oxy)hexadecyl)oxy)-5-oxopentyl)propane-1,3-diyldioctanoate, 2-(5-((4-((((1-ethylpiperidin-3-yl)methoxy)carbonyl)oxy)hexadecyl)oxy)-5-oxopentyl)propane-1,3-diyldioctanoate, 2-(5-((4-(((((R)-1-methylpyrrolidin-3-yl)oxy)carbonyl)oxy)hexadecyl)oxy)-5-oxopentyl)propane-1,3-diyldioctanoate, 2-(5-((4-(((((S)-1-methylpyrrolidin-3-yl)oxy)carbonyl)oxy)hexadecyl)oxy)-5-oxopentyl)propane-1,3-diyldioctanoate, 2-(5-oxo-5-((4-(((S)-pyrrolidine-2-carbonyl)oxy)hexadecyl)oxy)pentyl)propane-1,3-diyldioctanoate, 2-(5-((4-((1,3-dimethylpyrrolidine-3-carbonyl)oxy)hexadecyl)oxy)-5-oxopentyl)propane-1,3-diyldioctanoate, 2-(5-((4-((1,4-dimethylpiperidine-4-carbonyl)oxy)hexadecyl)oxy)-5-oxopentyl)propane-1,3-diyldioctanoate, 4,4-bis(octyloxy)butyl(3-(diethylamino)propyl)pentadecane-1,3-diyldicarbonate, 3-(((3-(diethylamino)propoxy)carbonyl)oxy)pentadecyl4,4-bis((2-propylpentyl)oxy)butanoate, ((2-((((3-(diethylamino)propoxy)carbonyl)oxy)methyl)-1,4-phenylene)bis(oxy))bis(octane-8,1-diyl)bis(decanoate), 4,4-bis(octyloxy)butyl5-(((3-(diethylamino)propoxy)carbonyl)oxy)heptadecanoate, 6-((6,6-bis(octyloxy)hexanoyl)oxy)-4-(((3-(diethylamino)propoxy)carbonyl)oxy)hexyloctanoate, (12Z,15Z)-3-(((3-(diethylamino)propoxy)carbonyl)oxy)henicosa-12,15-dien-1-yl6,6-bis(octyloxy)hexanoate, 3-(((3-(diethylamino)propoxy)carbonyl)oxy)tridecyl6,6-bis(octyloxy)hexanoate, 3-(((3-(diethylamino)propoxy)carbonyl)oxy)undecyl6,6-bis(octyloxy)hexanoate, 3-(((3-(diethylamino)propoxy)carbonyl)oxy)pentadecyl5-(4,6-diheptyl-1,3-dioxan-2-yl)pentanoate, 3-((5-(diethylamino)pentanoyl)oxy)pentadecyl6,6-bis(octyloxy)hexanoate, 1-((6,6-bis(octyloxy)hexanoyl)oxy)pentadecan-3-yl1,4-dimethylpiperidine-4-carboxylate, 3-((3-(1-methylpiperidin-4-yl)propanoyl)oxy)pentadecyl6,6-bis(octyloxy)hexanoate, 1-((6,6-bis(octyloxy)hexanoyl)oxy)pentadecan-3-yl1,3-dimethylpyrrolidine-3-carboxylate, 3-(((3-(diethylamino)propoxy)carbonyl)oxy)pentadecyl4,4-bis((2-ethylhexyl)oxy)butanoate, 2-(((1,3-dimethylpyrrolidine-3-carbonyl)oxy)methyl)propane-1,3-diylbis(8-(octanoyloxy)octanoate), ((2-((((3-(dimethylamino)propoxy)carbonyl)oxy)methyl)-1,4-phenylene)bis(oxy))bis(octane-8,1-diyl)bis(decanoate), (2R)-1-((6,6-bis(octyloxy)hexanoyl)oxy)pentadecan-3-ylpyrrolidine-2-carboxylate, (2S)-1-((6,6-bis(octyloxy)hexanoyl)oxy)pentadecan-3-yl1-methylpyrrolidine-2-carboxylate, (2R)-1-((6,6-bis(octyloxy)hexanoyl)oxy)pentadecan-3-yl1-methylpyrrolidine-2-carboxylate, 3-(((3-(dimethylamino)propoxy)carbonyl)oxy)pentadecyl6,6-bis((3-ethylpentyl)oxy)hexanote, 3-(((3-(dimethylamino)propoxy)carbonyl)oxy)pentadecyl6,6-bis((2-propylpentyl)oxy)hexanoate, 3-(((3-(diethylamino)propoxy)carbonyl)oxy)pentadecyl6,6-bis((2-propylpentyl)oxy)hexanoate, 3-(((2-(diethylamino)ethoxy)carbonyl)oxy)pentadecyl6,6-bis(octyloxy)hexanoate, 3-(((3-morpholinoproproxy)carbonyl)oxy)pentadecyl6,6-bis(octyloxy)hexanoate, 3-((((1-methylpiperidin-4-yl)methoxy)carbonyl)oxy)pentadecyl6,6-bis(octyloxy)hexanoate, 3-(((3-(4-methylpiperazin-1-yl)propoxy)carbonyl)oxy)pentadecyl6,6-bis(octyloxy)hexanoate, 3-(((3-(diethylamino)propoxy)carbonyl)oxy)pentadecyl4,4-bis(octyloxy)butanoate, 2-(((4-(dimethylamino)butanoyl)oxy)methyl)-2-((dodecanoyloxy)methyl)propane-1,3-diyl(9Z,9′Z)bis-tetradec-9-enoate, (9Z,9′Z,12Z,12′Z)-2-(((4-(dimethylamino)butanoyl)oxy)methyl)propane-1,3-diylbis(octadeca-9,12-dienoate), 3-(((4-(diethylamino)butoxy)carbonyl)oxy)pentadecyl6,6-bis(octyloxy)hexanote, 3-(((3-(piperazin-1-yl)propoxy)carbonyl)oxy)pentadecyl6,6-bis(octyloxy)hexanoate, 3-(((3-piperidin-1-yl)propoxy)carbonyl)oxy)pentadecyl6.6-bis(octyloxy)hexanoate, 3-(((3-(dimethylamino)propoxy)carbonyl)oxy)pentadecyl4,4-bis(octyloxy)butanoate, (9Z,9′Z,12Z,12′Z)-2-(9-dodecyl-2-methyl-7,12-dioxo-6,8,13-trioxa-2-azatetradecan-14-yl)propane-1,3-diylbis(octadeca-9,12-dienoate), (9Z,12Z)-10-dodecyl-3-ethyl-14-(2-((9Z,12Z)-octadeca-9,12-dienoyloxy)ethyl)-8,13-dioxo-7,9-dioxa-3,14-diazahexadecan-16-yloctadeca-9,12-dienoate, 2-((2-(((3-(diethylamino)propoxy)carbonyl)oxy)tetradecanoyl)oxy)propane-1,3-diyldioctanoate, 2-(9-dodecyl-2-methyl-7,13-dioxo-6,8,12-trioxa-2-azanonadecan-19-yl)propane-1,3-diyldioctanoate, 2-((decanoyloxy)methyl)-2-(((4-(dimethylamino)butanoyl)oxy)methyl)propane-1,3-diyl(9Z,9′Z)bis-tetradec-9-enoate, (9Z,9′Z,12Z,12′Z)-2-(((3-morpholinopropanoyl)oxy)methyl)propane-1,3-diylbis(octadeca-9,12-dienoate), 3-(Dimethylamino)propylcarbonate(6Z,9Z,28Z,31Z)-heptatriconta-6,9,28,31-tetraen-19-yl, 2,5-bis((9Z,12Z)-octadeca-9,12-dien-1-yloxy)benzyl4-(dimethylamino)butanoate, 2-(10-dodecyl-3-ethyl-8,14-dioxo-7,9,13-trioxa-3-azaoctadecan-18-yl)propane-1,3-diyldioctanoate, (9Z,9′Z,12Z,12′Z)-2-(((1,3-dimethylpyrrolidine-3-carbonyl)oxy)methyl)propane-1,3-diylbis(octadeca-9,12-dienoate), ((5-((dimethylamino)methyl)benzene-1,2,3-triyl)tris(oxy))tris(decane10,1-diyl)trioctanoate, 0′, 0-(((5-((dimethylamino)methyl)-1,3-phenylene)bis(oxy))bis(propane-3,1-diyl))9-dioctyldinonanedioate, (9Z,12Z)-3-(3-((dimethylamino)methyl)-5-(3-((3-octylundecanoyl)oxy)propoxy)phenoxy)propyloctadeca-9,12-dienoate, ((((5-((dimethylamino)methyl)-1,3-phenylene)bis(oxy))bis(propane-3,1-diyl))bis(oxy))bis(4-oxobutane-4,1-diyl)bis(decanoate), (R)-4-(3-((R)-3,4-bis(octanoyloxy)butoxy)-5-((dimethylamino)methyl)phenoxy)butane-1,2-diyldioctanoate, (S)-4-(3-((S)-3,4-bis(octanoyloxv)butoxv)-5-((dimethylamino)methyl)phenoxy)butane-1,2-diyldioctanoate, (R)-4-(3-((S)-3,4-bis(octanoyloxy)butoxy)-5-((dimethylamino)methyl)phenoxy)butane-1,2-diyldioctanoate, 4,4′-((5-((dimethylamino)methyl)-1,3-phenylene)bis(oxy))bis(butane1,2-diyl)tetraoctanoate, didodecyl6,6′-((5-((dimethylamino)methyl)-1,3-phenylene)bis(oxy))dihexanoate, di((9Z,12Z)-octadeca-9,12-dien-1-yl)5, 5′-((5-((dimethylamino)methyl)-1,3-phenylene)bis(oxy))dipentanoate, (((5-((dimethylamino)methyl)-1,3-phenylene)bis(methylene))bis(oxy))bis(6-oxohexane-6,1-diyl)bis(decanoate), (5-((dimethylamino)methyl)-1,3-phenylene)bis(methylene)bis(8-(octanoyloxy)octanoate), (5-((dimethylamino)methyl)-1,3-phenylene)bis(methylene)bis(10-(octanoyloxy)decanoate), (((5-((dimethylamino)methyl)-1,3-phenylene)bis(methylene))bis(oxy))bis(6-oxohexane-6,1-diyl)dioctanoate, (((5-((dimethylamino)methyl)-1,3-phenylene)bis(methylene))bis(oxy))bis(8-oxooctane-8,1-diyl)bis(decanoate), (9Z,9′Z,12Z,12′Z)-(((5-((dimethylamino)methyl)-1,3-phenylene)bis(methylene))bis(oxy))bis(4-oxobutane-4,1-diyl)bis(octadeca-9,12-dienoate), O′, O-((5-((dimethylamino)methyl)-1,3-phenylene)bis(methylene))8-dinonyldioctanedioate, O, O′-((5-((dimethylamino)methyl)-1,3-phenylene)bis(methylene))bis(10-(octanoyloxy)decyl)disuccinate, 0,0′-((5-((dimethylamino)methyl)-1,3-phenylene)bis(methylene))di((9Z,12Z)-octadeca-9,12-dien-1-yl) disuccinate, (9Z,9′Z,12Z,12′Z)-(5-((((3-(diethylamino)propoxy)carbonyl)oxy)methyl)-1,3-phenylene)bis(methylene)bis(octadeca-9,12-dienoate), (9Z,12Z)-4-(3-((dimethylarnino)methyl)-5-(4-(oleoyloxy)butoxy)phenoxy)butyloctadeca-9,12-dienoate, (9Z,9′Z,12Z,12′Z,15Z,15′Z)-((5-((dimethvlamino)methyl)-1,3-phenylene)bis(oxy))bis(butane-4,1-diyl)bis(octadeca-9,12,15-trienoate), ((5-((dimethylamino)methyl)-1,3-phenylene)bis(oxy))bis(butane-4,1-diyl)ditetradecanoate, (Z)-((5-((dimethylamino)methyl)-1,3-phenylene)bis(oxy))bis(butane-4,1-diyl)dioleate, ((5-((dimethylamino)methyl)-1,3-phenylene)bis(oxy))bis(hexane-6,1-diyl)didodecanoate, (9Z,9′Z,12Z,12′Z)-((((5-((diethylamino)methyl)-1,3-phenylene)bis(oxy))bis(ethane-2,1-diyl))bis(oxy))bis(ethane-2,1-diyl)bis(octadeca-9,12-dienoate), didecyl8,8′-((5-((dimethylamino)methyl)-1,3-phenylene)bis(oxy))dioctanoate, ((5-((dimethylamino)methyl)-1,3-phenylene)bis(oxy))bis(propane-3,1-diyl)bis(3-octylundecanoate), (9Z.9′Z.12Z.12′Z)-((5-((diethvlamino)methvn-2-methvl-1.3-phenylene)bis(oxy))bis(butane-4,1-diyl)bis(octadeca-9,12-dienoate), ((5-((dimethylamino)methyl)-1,3-phenylene)bis(oxy))bis(octane-8,1-diyl)didodecanoate, ((5-((dimethylamino)methyl)-1,3-phenylene)bis(oxy))bis(octane-8,1-diyl)bis(decanoate), (9Z,9′Z,12Z,12′Z)-((5-((dimethvlarnino)methen-2-methyl-1,3-phenylene)bis(oxy))bis(butane-4,1-diyl)bis(octadeca-9,12-dienoate), (8Z,8′Z)-((5-((dimethylamino)methyl)-1,3-phenylene)bis(oxy))bis(hexane-bis(dodec-8-enoate), (9Z,9′Z,12Z,12′Z)-((5-((3-hydroxyazetidin-1-yl)methyl)-1,3-phenylene)bis(oxy))bis(butane-4,1-diyl)bis(octadeca-9,12-dienoate), ((5-((dimethylamino)methyl)-1,3-phenylene)bis(oxy))bis(hexane-6,1-diyl)dioctanoate, ((5-((dimethylamino)methyl)-1,3-phenylene)bis(oxy))bis(hexane-6,1-diyl)bis(decanoate), (9Z.9′Z.12Z.12′Z)-((5-((dimethvlamino)methvn-1.3-phenylene)bis(oxy))bis(octane-8,1-diyl)bis(octadeca-9,12-dienoate), (9Z,9′Z,12Z,12′Z)-((5-((dimethvlamino)methyl)-1,3-phenylene)bis(oxy))bis(hexane-6,1-diyl)bis(octadeca-9,12-dienoate), ((5-((dimethylamino)methyl)-1,3-phenylene)bis(oxy))bis(decane-10,1-diyl)dihexanoate, ((5-((dimethylamino)methyl)-1,3-phenylene)bis(oxy))bis(decane-10,1-diyl)dioctanoate, ((5-((dimethylamino)methyl)-1,3-phenylene)bis(oxy))bis(octane-8,1-diyl)dioctanoate, ((5-((dimethylamino)methyl)-1,3-phenylene)bis(oxy))bis(octane-8,1-diyl)dihexanoate, (9Z,9′Z,12Z,12′Z)-((5-((dimethvlamino)methyl)-1,3-phenylene)bis(oxy))bis(ethane-2,1-diyl)bis(octadeca-9,12-dienoate), (9Z,9′Z,12Z,12′Z)-((5-((dimethylamino)methyl)-1,3-phenylene)bis(oxy))bis(propane-3,1-diyl)bis(octadeca-9,12-dienoate), (9Z,9′Z,12Z,12′Z)-((5-((dimethylamino)methyl)-1,3-phenylene)bis(oxy))bis(butane-4,1-diyl)bis(octadeca-9,12-dienoate), (5-((dimethylamino)methyl)-1,3-phenylene)bis(methylene)ditridecanoate, (9Z,9′Z,12Z,12′Z)-(5-((dimethylamino)methyl)-1,3-phenylene)bis(methylene)bis(octadeca-9,12-dienoate), (2,6-bis((9Z,12Z)-octadeca-9,12-dien-1-yloxy)pyridin-4-yl)methyl3-(dimethylamino)propanoate, (9Z,9′Z,12Z,12′Z)-5-(((3-(dimethylamino)propanoyl)oxy)methyl)-1,3-phenylenebis(octadeca-9,12-dienoate), 1-(3,5-bis((9Z,12Z)-octadeca-9,12-dien-1-yloxy)phenyl)-N,Ndimethylmethanamine, 3,5-bis((9Z,12Z)-octadeca-9,12-dien-1-yloxy)benzyl3-(dimethylamino)propanoate, 1-(3,5-bis(4,4-bis(octyloxy)butoxy)phenyl)-N,N-dimethylmethanamine, ((((5-((dimethylamino)methyl)-1,3-phenylene)bis(oxy))bis(butane-4,1-diyl))bis(oxy))bis(propane-3,2,1-triyl)tetraoctanoate, ((5-(((4-(dimethylamino)butanoyl)oxy)methyl)-1,3-phenylene)bis(oxy))bis(octane-8,1-diyl)bis(decanoate), ((5-(((3-(dimethylamino)propanoyl)oxy)methyl)-1,3-phenylene)bis(oxy))bis(octane-8,1-diyl)bis(decanoate), (9Z,9′Z,12Z,12′Z)-((5-(3-morpholinopropyl)-1,3-phenylene)bis(oxy))bis(butane4,1-diyl)bis(octadeca-9,12-dienoate), (9Z,9′Z,12Z,12′Z)-((5-(3-(dimethvlamino)propyl)-1,3-phenylene)bis(oxy))bis(butane-4,1-diyl)bis(octadeca-9,12-dienoate), (9Z,9′Z,12Z,12′Z)-((5-(3-(piperidin-1-yl)propyl)-1,3-phenylene)bis(oxy))bis(butane-4,1-diyl)bis(octadeca-9,12-dienoate), (5-((dimethylamino)methyl)-1,3-phenylene)bis(methylene)bis(9-pentyltetradecanoate), (5-((dimethylamino)methyl)-1,3-phenylene)bis(methylene)bis(7-hexyltridecanoate), (5-((dimethylamino)methyl)-1,3-phenylene)bis(methylene)bis(5-heptyldodecanoate), ((5-((dimethylamino)methyl)-1,3-phenylene)bis(oxy))bis(butane-4,1-diyl)bis(3-octylundecanoate), ((5-((dimethylamino)methyl)-1,3-phenylene)bis(oxy))bis(butane-4,1-diyl)bis(5-heptyldodecanoate), ((5-((dimethylamino)methyl)-1,3-phenylene)bis(oxy))bis(butane-4,1-diyl)bis(9-pentyltetradecanoate), ((5-((dimethylamino)methyl)-1,3-phenylene)bis(oxy))bis(butane-4,1-diyl)bis(7-hexyltridecanoate), (9Z,9′Z,12Z,12′Z)-((5-(pyrrolidin-1-ylmethyl)-1,3-phenylene)bis(oxy))bis(butan4,1-diyl)bis(octadeca-9,12-dienoate), (((5-((dimethylamino)methyl)-1,3-phenylene)bis(oxy))bis(methylene))bis(propane-3,2,1-triyl)tetraoctanoate, (((5-((dimethylamino)methyl)-1,3-phenylene)bis(oxy))bis(butane-4,1-diyl))bis(propane-3,2,1-triyl)tetraoctanoate, (9Z.12Z)-4-(3-((dimethvlamino)methvn-5-(4-((3-octylundecanoyl)oxy)butoxy)phenoxy)butyloctadeca-9,12-dienoate, bis(1,3-bis(octanoyloxy)propan-2-yl)0,0′-((5-((dimethylamino)methyl)-1,3-phenylene)bis(methylene))disuccinate, (5-((dimethylamino)methyl)-1,3-phenylene)bis(methylene)bis(6-(((nonyloxy)carbonyl)oxy)hexanoate), 2-(3-(4-(5-((dimethylamino)methyl)-2-methyl-3-((9Z,12Z)-octadeca9,12-dien-1-yloxy)phenoxy)butoxy)-3-oxopropyl)propane-1,3-diyldihexanoate, 3-((dimethylamino)methyl)-5-(((8-(octanoyloxy)octanoyl)oxy)methyl)benzyl3-octylundecanoate, ((5-((diethylamino)methyl)benzene-1,2,3-triyl)tris(oxy))tris(decane-10,1-diyl)trioctanoate, 1-(3,5-bis((Z)-octadec-9-en-1-yloxy)phenyl)-N,N-dimethylmethanamine, N‘-methyl-N’,N″.N″-tris((2E.6E)-3.7.11-trimethyldodeca-2.6.10-trien-1-vnpropane-1,3-diamine, 1,17-bis(2-((2-pentylcyclopropyl)methyl)cyclopropyl)heptadecan-9-yl4-(dimethylamino)butanoate, ethyl(7Z)-17-{[4-(dimethylamino)butanoyl]oxy}hexacos-7-enoate, (Z)-methyl6-(2-(dimethylamino)-3-(octadec-9-en-1-yloxy)propoxy)hexanoate, 2-(Didodecylamino)-1-(4-(N-(2-(dinonylamino)ethyl)-N-dodecylglycyl)piperazin-1-yl)ethan-1-one, 3-((3-(1-(3-((2-(Dinonylamino)ethyl)(nonyl)amino)propanoyl)piperidin-4-yl)propyl)(nonyl)amino)propylhexanoate, 3-((3-(4-(3-((2-(Dinonylamino)ethyl)(nonyl)amino)propanoyl)piperazin-1-yl)-3-oxopropyl)(nonyl)amino)propylhexanoate, 3-((2-(Dinonylamino)ethyl)(nonyl)amino)-1-(4-(3-(dinonylamino)propyl)piperidin-1-yl)propan-1-one, Pentyl4-((3-(1-(3-((2-(dinonylamino)ethyl)(nonyl)amino)propanoyl)piperidin-4-yl)propyl)(nonyl)amino)butano, Pentyl4-((2-(1-(N-(2-(dinonylamino)ethyl)-N-nonylglycyl)piperidin-4-yl)ethyl)(nonyl)amino)butanoate, Pentyl4-(((1-(N-(2-(dinonylamino)ethyl)-N-nonylglycyl)pyrrolidin-3-yl)methyl)(nonyl)amino)butanoate, Pentyl4-((2-(1-(N-(2-(dinonylamino)ethyl)-N-nonylglycyl)pyrrolidin-3-yl)ethyl)(nonyl)amino)butanoate, Pentyl4-((2-(1-(N-(2-(dinonylamino)ethyl)-N-nonylglycyl)piperidin-3-yl)ethyl)(nonyl)amino)butanoate, 2-(Didodecylamino)-1-(4-(N-(2-(dinonylamino)ethyl)-N-nonylglycyl)piperazin-1-yl)ethan-1-one, 2-((2-(Dinonylamino)ethyl)(nonyl)amino)-1-(3-(2-(dinonylamino)ethyl)piperidin-1-yl)ethan-1-one, Dipentyl 4,4′-((2-(4-(N-(2-(dinonylamino)ethyl)-N-nonylglycyl)piperazin-1-yl)-2-oxoethyl)azanediyl)dibutyrate, Pentyl 4-(nonyl(2-(4-(N-nonyl-N-(2-(nonyl(4-oxo-4-(penlyloxy)buryl)amino)ethyl)glycyl)piperazin-1-yl)-2-oxoethyl)amino)butanoate, 2-((2-(Dinonylamino)ethyl)(nonyl)amino)-1-(3-((dinonylamino)methyl)pyrrolidin-1-yl)ethan-1-one, 2-((2-(Didodecylamino)ethyl)(dodecyl)amino)-1-(4-(dinonylglycyl)piperazin-1-yl)ethan-1-one, 2-((2-(Dinonylamino)ethyl)(nonyl)amino)-1-(3-(2-(dinonylamino)ethyl)pyrrolidin-1-yl)ethan-1-one, Pentyl4-((3-(4-(3-((2-(dinonylamino)ethyl)(nonyl)amino)propanoyl)piperazin-1-yl)-3-oxopropyl)(nonyl)amino)butanoate, 3-((2-(1-(N-(2-(Dinonylamino)ethyl)-N-nonylglycyl)piperidin-4-yl)ethyl)(nonyl)amino)propylhexanoate, Butyl5-((2-(1-(N-(2-(dinonylamino)ethyl)-N-nonylglycyl)piperidin-4-yl)ethyl)(nonyl)amino)pentanoate, 2-((2-(Didodecylamino)ethyl)(nonyl)amino)-1-(4-(dinonylglycyl)piperazin-1-yl)ethan-1-one, Propyl6-((2-(1-(N-(2-(dinonylamino)ethyl)-N-nonylglycyl)piperidin-4-yl)ethyl)(nonyl)amino)hexanoate, Ethyl7-((2-(1-(N-(2-(dinonylamino)ethyl)-N-nonylglycyl)piperidin-4-yl)ethyl)(nonyl)amino)heptanoate, Methyl8-((2-(1-(N-(2-(dinonylamino)ethyl)-N-nonylglycyl)piperidin-4-yl)ethyl)(nonyl)amino)octanoate, 3-((2-(4-(N-(2-(Dinonylamino)ethyl)-N-nonylglycyl)piperazin-1-yl)-2-oxoethyl)(nonyl)amino)propylhexanoate, Butyl5-((2-(4-(N-(2-(dinonylamino)ethyl)-N-nonylglycyl)piperazin-1-yl)-2-oxoethyl)(nonyl)amino)pentanoate, Propyl 6-((2-(4-(N-(2-(dinonylamino)ethyl)-N-nonylglycyl)piperazin-2-oxoethyl)(nonyl)amino)hexanoate, Ethyl7-((2-(4-(N-(2-(dinonylamino)ethyl)-N-nonylglycyl)piperazin-1-yl)-2-oxoethyl)(nonyl)amino)heptanoate, 3-(Dinonylamino)-1-(4-(3-((2-(dinonylamino)ethyl)(nonyl)amino)propanoyl)piperazin-1-yl)propan-1-one, 2-((2-(Dinonylamino)ethyl)(nonyl)amino)-1-(4-(ditetradecylglycyl)piperazin-1-yl)ethan-1-one, 2-(Dinonylamino)-1-(4-(2-((2-(dinonylamino)ethyl)(nonyl)amino)ethyl)piperidin-1-yl)ethan-1-one, 2-(Dinonylamino)-1-(4-(N-(2-(dinonylamino)ethyl)-N-dodecylglycyl)piperazin-1-yl)ethan-1-one, 2-((2-(Dinonylamino)ethyl)(nonyl)amino)-1-(4-(2-(dinonylamino)ethyl)piperidin-1-yl)ethan-1-one, Methyl8-((2-(4-(dinonylglycyl)piperazin-1-yl)-2-oxoethyl)(2-((8-methoxy-8-oxooctyl)(nonyl)amino)ethyl)amino)octanoate, Methyl8-((2-(dinonylamino)ethyl)(2-(4-(dinonylglycyl)piperazin-1-yl)-2-oxoethyl)amino)octanoate, Methyl 8-((2-((2-(4-(dinonylglycyl)piperazin-1-yl)-2-oxoethyl)(nonyl)amino)ethyl)(nonyl)amino)octanoate, Pentyl 4-((2-(4-(N-(2-(dinonylamino)ethyl)-N-nonylglycyl)piperazin-2-oxoethyl)(nonyl)amino)butanoate, Methyl8-((2-(4-(N-(2-(dinonylamino)ethyl)-N-nonylglycyl)piperazin-1-yl)-2-oxoethyl)(nonyl)amino)octanoate, 2-((2-(Didodecylamino)ethyl)(dodecyl)amino)-1-(5-(dinonylglycyl)-2,5-diazabicyclo[2.2.1]heptan-2-yl)ethan-1-one3, 2-(Dinonylamino)-1-(5-(N-(2-(dinonylamino)ethyl)-N-nonylglycyl)-2,5-diazabicyclo[2.2.1]heptan-2-yl)ethan-1-one, N1,N1,N2-Tri((9Z,12Z)-octadeca-9,12-dien-1-yl)-N2-(2-(piperazin-1-yl)ethyl)ethane-1,2-diamine, N1,N1,N2-Tri((Z)-octadec-9-en-1-yl)-N2-(2-(piperazin-1-yl)ethyl)ethane-1,2-diamine, 2-(Dinonylamino)-1-(4-(N-(2-(dinonylamino)ethyl)-N-nonylglycyl)piperazin-1-yl)ethan-1-one, N1,N1,N2-Tridodecyl-N2-(2-(piperazin-1-yl)ethyl)ethane-1,2-diamine, N1,N1,N2-Trinonyl-N2-(2-(piperazin-1-yl)ethyl)ethane-1,2-diamine, N1,N1,N2-Trihexyl-N2-(2-(piperazin-1-yl)ethyl)ethane-1,2-diamine, N1-(2-(4-(2-(Didodecylamino)ethyl)piperazin-1-yl)ethyl)-N1,N2,N2-tri((9Z,12Z)-octadeca-9,12-dien-1-yl)ethane-1,2-diamine, N1-(2-(4-(2-(Didodecylamino)ethyl)piperazin-1-yl)ethyl)-N1,N2,N2-tri((Z)-octadec-9-en-1-yl)ethane-1,2-diamine, N1-(2-(4-(2-(Ditetradecylamino)ethyl)piperazin-1-yl)ethyl)-N1,N2,N2-tritetradecylethane-1,2-diamine, N1-(2-(4-(2-(Didodecylamino)ethyl)piperazin-1-yl)ethyl)-N1,N2,N2-tritetradecylethane-1,2-diamine, N1-(2-(4-(2-(Dinonylamino)ethyl)piperazin-1-yl)ethyl)-N1,N2,N2-tritetradecylethane-1,2-diamine, 2-(Didodecylamino)-1-(4-(2-((2-(didodecylamino)ethyl)(dodecyl)amino)ethyl)piperazin-1-yl)ethan-1-one, N1-(2-(4-(2-(Di((9Z,12Z)-octadeca-9,12-dien-1-yl)amino)ethyl)piperazin-1-yl)ethyl)-N1,N2,N2-tridodecylethane-1,2-diamine, N1-(2-(4-(2-(Di((Z)-octadec-9-en-1-yl)amino)ethyl)piperazin-1-yl)ethyl)-N1,N2,N2-tridodecylethane-1,2-diamine, N1,N1,N2-Tridodecyl-N2-(2-(4-(2-(dodecyl((9Z,12Z)-octadeca-9,12-dien-1-yl)amino)ethyl)piperazin-1-yl)ethyl)ethane-1,2-diamine, N1-(2-(4-(2-(Ditetradecylamino)ethyl)piperazin-1-yl)ethyl)-N1,N2,N2-tridodecylethane-1,2-diamine, N1-(2-(4-(2-(Di((Z)-dodec-6-en-1-yl)amino)ethyl)piperazin-1-yl)ethyl)-N1,N2,N\hat{ }-tridodecylethane-1,2-diamine, (Z)—N1-(2-(4-(2-(Dodec-6-en-1-yl(dodecyl)amino)ethyl)piperazin-1-yl)ethyl)-N,N2,N2-tridodecylethane-1,2-diamine, N1-(2-(4-(2-(Dinonylamino)ethyl)piperazin-1-yl)ethyl)-N1,N2,N2-tridodecylethane-1,2-diamine, N1-(2-(4-(2-(Dioctylamino)ethyl)piperazin-1-yl)ethyl)-N1,N2,N2-tridodecylethane-1,2-diamine, N1-(2-(4-(2-(Dihexylamino)ethyl)piperazin-1-yl)ethyl)-N1,N2,N2-tridodecylethan-1,2-diamine, N1-(2-(4-(2-(Ditetradecylamino)ethyl)piperazin-1-yl)ethyl)-N1,N2,N2-trinonylethane-1,2-diamine, 2-((2-(Didodecylamino)ethyl)(dodecyl)amino)-1-(4-(2-(didodecylamino)ethyl)piperazin-1-yl)ethan-1-one, N1-(2-(4-(2-(Didodecylamino)ethyl)piperazin-1-yl)ethyl)-N1,N2,N2-trinonylethane-1,2-diamine, N1-(2-(4-(2-(Dinonylamino)ethyl)piperazin-1-yl)ethyl)-N1,N2,N2-trinonylethane-1,2-diamine, N1-(2-(4-(2-(Didodecylamino)ethyl)piperazin-1-yl)ethyl)-N1,N2,N2-trihexylethane-1,2-diamine, Dimethy112,12′-((2-(4-(2-((2-(didodecylamino)ethyl)(dodecyl)amino)ethyl)piperazin-1-yl)ethyl)azanediyl)didodecanoate, Methy112-((2-(4-(2-((2-(didodecylamino)ethyl)(dodecyl)amino)ethyl)piperazin-1-yl)ethyl)(dodecyl)amino)dodecanoate, Dipentyl 6,6′-((2-(4-(2-((2-(didodecylamino)ethyl)(dodecyl)amino)ethyl)piperazin-1-yl)ethyl)azanediyl)dihexanoate, Pentyl 6-((2-(4-(2-((2-(ditetradecylamino)ethyl)(tetradecyl)amino)ethyl)piperazin-1-yl)ethyl)(dodecyl)amino)hexanoate, Pentyl 6-((2-(4-(2-((2-(didodecylamino)ethyl)(dodecyl)amino)ethyl)piperazin-1-yl)ethyl)(dodecyl)amino)hexanoate, 2-(Didodecylamino)-1-(4-(N-(2-(didodecylamino)ethyl)-N-dodecylglycyl)piperazin-1-yl)ethan-1-one, 2-(Didodecylamino)-1-(4-(N-(2-(didodecylamino)ethyl)-N-nonylglycyl)piperazin-1-yl)ethan-1-one, 2-(Didodecylamino)-N-(2-(4-(2-(didodecylamino)ethyl)piperazin-1-yl)ethyl)-N-dodecylacetamide, ((2-((3,S′,4R)-3,4-dihydroxypyrrolidin-1-yl)acetyl)azanediyl)bis(ethane-2,1-diyl)(9Z,9′Z,12Z,12′Z)-bis(octadeca-9,12-dienoate), 2-amino-N,N-dihexadecyl-3-(1H-imidazol-5-yl)propanamide, (2-amino-N,N-dihexadecyl-3-(1H-imidazol-5-yl)propanamide, methyl(9Z)-19-[2-(dimethylamino)ethyl]heptacos-9-enoate, methyl8-(2-{9-[2-(dimethylamino)ethyl]octadecyl}cyclopropyl)octanoate, methyl(9Z)-19-[2-(dimethylamino)ethyl]octacos-9-enoate, ethyl8-(2-{ll-[(dimethylamino)methyl]heptadecyl}cyclopropyl)octanoate, ethyl8-(2-{ll-[(dimethylamino)methyl]octadecyl}cyclopropyl)octanoate, di((9Z,12Z)-octadeca-9,12-dien-1-yl)3-(((2-(dimethylamino)ethoxy)carbonyl)amino)pentanedioate, Heptyl6-((2-(1-(N-(2-(dinonylamino)ethyl)-N-nonylglycyl)piperidin-4-yl)ethyl)(tetradecyl)amino)hexanoate, ethyl8-(2-{ll-[(dimethylamino)methyl]nonadecyl}cyclopropyl)octanoate, Pentyl8-((2-(1-(N-(2-(dinonylamino)ethyl)-N-nonylglycyl)piperidin-4-yl)ethyl)(tetradecyl)amino)octanoate, ethyl8-(2-{ll-[(dimethylamino)methyl]icosyl}cyclopropyl)octanoate, ethyl 8-(2-{9-[(dimethylamino)methyl]pentadecyl}cyclopropyl)octanoate, 3-((2-(1-(N-(2-(Dinonylamino)ethyl)-N-nonylglycyl)piperidin-4-yl)ethyl)(tetradecyl)amino)propyldecanoate, Heptyl6-((2-(4-(N-(2-(dinonylamino)ethyl)-N-nonylglycyl)piperazin-1-yl)-2-oxoethyl)(tetradecyl)amino)hexanoate, ethyl8-(2-{9-[(dimethylamino)methyl]hexadecyl}cyclopropyl)octanoate, Pentyl8-((2-(4-(N-(2-(dinonylamino)ethyl)-N-nonylglycyl)piperazin-2-oxoethyl)(tetradecyl)amino)octanoate, ethyl8-(2-{9-[(dimethylamino)methyl]heptadecyl}cyclopropyl)octanoate, methyl6-(2-(8-(2-(dimethylamino)-3-(nonyloxy)propoxy)octyl)cyclopropyl)hexanoate, methyl(9Z)-21-(dimethylamino)heptacos-9-enoate, methyl(9Z)-21-{[4-(dimethylamino)butanoyl]oxy}heptacos-9-enoate, (2R)—N,N-dimethyl-1-[(9Z,12Z)-octadeca-9,12-dien-1-yloxy]dodecan-2-amine, (15Z,18Z)—N,N-dimethyltetracoda-15,18-dien-5-amine, ethyl8-(2-{9-[(dimethylamino)methyl]octadecyl}cyclopropyl)octanoate, 3-((2-(4-(N-(2-(Dinonylamino)ethyl)-N-nonylglycyl)piperazin-1-yl)-2-oxoethyl)(tetradecyl)amino)propyldecanoate, ethyl4-(2-{ll-[(dimethylamino)methyl]icosyl}cyclopropyl)butanoate, ethyl8-(2-{7-[(dimethylamino)methyl]hexadecyl}cyclopropyl)octanoate, 3-((3-(1-(3-((2-(Dinonylamino)ethyl)(nonyl)amino)propanoyl)piperidin-4-yl)propyl)(nonyl)amino)propylhexanoate, ethyl6-(2-{9-[(dimethylamino)methyl]pentadecyl}cyclopropyl)hexanoate, 3-((3-(4-(3-((2-(Dinonylamino)ethyl)(nonyl)amino)propanoyl)piperazin-1-yl)-3-oxopropyl)(nonyl)amino)propylhexanoate, ethyl6-(2-{9-[(dimethylamino)methyl]hexadecyl}cyclopropyl)hexanoate, 3-((2-(Dinonylamino)ethyl)(nonyl)amino)-1-(4-(3-(dinonylamino)propyl)piperidin-1-yl)propan-1-one, ethyl6-(2-{9-[(dimethylamino)methyl]heptadecyl}cyclopropyl)hexanoate, Pentyl 4-((2-(1-(N-(2-(dinonylamino)ethyl)-N-nonylglycyl)piperidin-4-yl)ethyl)(nonyl)amino)butanoate, ethyl6-(2-{9-[(dimethylamino)methyl]octadecyl}cyclopropyl)hexanoate, Pentyl 4-(((1-(N-(2-(dinonylamino)ethyl)-N-nonylglycyl)pyrrolidin-3-yl)methyl)(nonyl)amino)butanoate, ethyl(9Z)-21-[(dimethylamino)methyl]heptacos-9-enoate, Pentyl4-((2-(1-(N-(2-(dinonylamino)ethyl)-N-nonylglycyl)pyrrolidin-3-yl)ethyl)(nonyl)amino)butanoate, ethyl(9Z)-21-[(dimethylamino)methyl]octacos-9-enoate, ((2-((3,S′,4R)-3,4-dihydroxypyrrolidin-1-yl)acetyl)azanediyl)bis(ethane-2,l-diyl)(9Z,9′Z,12Z,12′Z)-bis(octadeca-9,12-dienoate), Pentyl 4-((2-(1-(N-(2-(dinonylamino)ethyl)-N-nonylglycyl)piperidin-3-yl)ethyl)(nonyl)amino)butanoate, ethyl(9Z)-21-[(dimethylamino)methyl]nonacos-9-enoate, methyl6-(2-(8-(2-(dimethylamino)-3-(heptyloxy)propoxy)octyl)cyclopropyl)hexanoate, methyl(9Z)-21-{[4-(dimethylamino)butanoyl]oxy}octacos-9-enoate, methyl(9Z)-21-(dimethylamino)octacos-9-enoate, 2-(Didodecylamino)-1-(4-(N-(2-(dinonylamino)ethyl)-N-nonylglycyl)piperazin-1-yl)ethan-1-, (2S)—N.N-dimethyl-1-[(9Z,12Z)-octadeca-9,12-dien-1-yloxy]nonan-2-amine, (18Z,21Z)—N,N-dimethylheptacosa-18,21-dien-10-amine, ethyl(9Z)-21-[(dimethylamino)methyl]triacont-9-enoate, ethyl(9Z)-19-[(dimethylamino)methyl]pentacos-9-enoate, ethyl(9Z)-19-[(dimethylamino)methyl]hexacos-9-enoate, ethyl(9Z)-19-[(dimethylamino)methyl]heptacos-9-enoate, ethyl(9Z)-19-[(dimethylamino)methyl]octacos-9-enoate, ethyl(5Z)-17-[(dimethylamino)methyl]hexacos-5-enoate, ethyl(9Z)-17-[(dimethylamino)methyl]hexacos-9-enoate, 2-((2-(Dinonylamino)ethyl)(nonyl)amino)-1-(3-(2-(dinonylamino)ethyl)piperidin-1-yl)ethan-1-one, ethyl(7Z)-17-[(dimethylamino)methyl]tricos-7-enoate, Dipentyl4,4′-((2-(4-(N-(2-(dinonylarnino)ethyl)-N-nonylglycyl)piperazin-1-yl)-2-oxoethyl)azanediyl)dibutyrate, Pentyl4-(nonyl(2-(4-(N-nonyl-N-(2-(nonyl(4-oxo-4-(pentyloxy)butyl)amino)ethyl)glycyl)piperazin-1-yl)-2-oxoethyl)amino)butanoate, ethyl(7Z)-17-[(dimethylamino)methyl]tetracos-7-enoate, ethyl(7Z)-17-[(dimethylamino)methyl]pentacos-7-enoate, 2-((2-(Dinonylamino)ethyl)(nonyl)amino)-1-(3-((dinonylamino)methyl)pyrrolidin-1-yl)ethan-1-one, trans-3-[(3}7-dimethyloctyl)oxy]-1-methyl-4˜[(9Z,12Z)-octadeca-9512-dien-1-yloxyjpyrrolidine, methyl6-(2-(8-(2-(dimethylamino)-3-(hexyloxy)propoxy)octyl)cyclopropyl)hexanoate, methyl(9Z)-21-{[4-(dimethylamino)butanoyl]oxy}nonacos-9-enoate, methyl(9Z)-21-(dimethylamino)nonacos-9-enoate, (2S)—N,N-dimethyl-1-[(9Z,12Z)-octadeca-9,12-dien-1-yloxy]tridecan-2-amine, (15Z,18Z)—N,N-dimethyltetracosa-15,18-dien-7-amine, ethyl(7Z)-17-[(dimethylamino)methyl]hexacos-7-enoate, 2-((2-(Dinonylamino)ethyl)(nonyl)amino)-1-(3-(2-(dinonylamino)ethyl)pyrrolidin-1-yl)ethan-1-one, methyl6-(2-{ll-[(dimethylamino)methyl]icosyl}cyclopropyl)hexanoate, methyl10-(2-{7-[(dimethylamino)methyl]hexadecyl}cyclopropyl)decanoate, methyl8-(2-{11-[(dimethylamino)methyl]heptadecyl}cyclopropyl)octanoate, methyl8-(2-{11-[(dimethylamino)methyl]octadecyl}cyclopropyl)octanoate, methyl8-(2-{ll-[(dimethylamino)methyl]nonadecyl}cyclopropyl)octanoate, methyl8-(2-{11-[(dimethylamino)methyl]icosyl}cyclopropyl)octanoate, Pentyl4-((3-(4-(3-((2-(dinonylamino)ethyl)(nonyl)amino)propanoyl)piperazin-1-yl)-3-oxopropyl)(nonyl)amino)butanoate, methyl8-(2-{9-[(dimethylamino)methyl]pentadecyl}cyclopropyl)octanoate, methyl8-(2-{9-[(dimethylamino)methyl]hexadecyl}cyclopropyl)octanoate, 3-((2-(1-(N-(2-(Dinonylamino)ethyl)-N-nonylglycyl)piperidin-4-yl)ethyl)(nonyl)amino)propylhexanoate, methyl8-(2-{9-[(dimethylamino)methyl]heptadecyl}cyclopropyl)octanoate, methyl8-(2-(dimethylamino)-3-((6-((2-octylcyclopropyl)methoxy)-6-oxohexyl)oxy)propoxy)octanoate, Butyl5-((2-(1-(N-(2-(dinonylamino)ethyl)-N-nonylglycyl)piperidin-4-yl)ethyl)(nonyl)amino)pentanoate, trans-1-methyl-3-[(12Z)-octadec-12-en-1-yloxy]-4-(octyloxy)pyrrolidine, methyl(9Z)-21-{[4-(dimethylamino)butanoyl]oxy}triacont-9-enoate, methyl(9Z)-21-(dimethylamino)triacont-9-enoate, 1-((2R,3 S,5R)-3-(bis(hexadecyloxy)methoxy)-5-(5-methyl-2,4-dioxo-3,4-dihydropyrimidin-1(2H)-yl)tetrahydrofumethanesulfonate, (Z)-methy116-(3-(decyloxy)-2-(dimethylamino)propoxy)hexadec-7-enoate, (2S)-1-[(9Z,12Z)-octadeca-9,12-dien-1-yloxy]nonan-2-amine, (14Z,17Z)—N,N-dimethyltricosa-14,17-dien-6-amine, Propyl6-((2-(1-(N-(2-(dinonylamino)ethyl)-N-nonylglycyl)piperidin-4-yl)ethyl)(nonyl)amino)hexanoate, methyl7-(2-(dimethylamino)-3-((6-((2-octylcyclopropyl)methoxy)-6-oxohexyl)oxy)propoxy)heptanoate, methyl(7Z)-19-[(dimethylamino)methyl]octacos-7-enoate, methyl(HZ)-19-[(dimethylamino)methyl]octacos-li-enoate, Ethyl7-((2-(1-(N-(2-(dinonylamino)ethyl)-N-nonylglycyl)piperidin-4-yl)ethyl)(nonyl)amino)heptanoate, (2-octylcyclopropyl)methyl6-(2-(dimethylamino)-3-((5-methoxy-5-oxopentyl)oxy)propoxy)hexanoate, Methyl8-((2-(1-(N-(2-(dinonylamino)ethyl)-N-nonylglycyl)piperidin-4-yl)ethyl)(nonyl)amino)octanoate, methyl(9Z)-21-[(dimethylamino)methyl]heptacos-9-enoate, (2-octylcyclopropyl)methyl6-(2-(dimethylamino)-3-(4-methoxy-4-oxobutoxy)propoxy)hexanoate, methyl(9Z)-21-[(dimethylamino)methyl]octacos-9-enoate, 3-((2-(4-(N-(2-(Dinonylamino)ethyl)-N-nonylglycyl)piperazin-1-yl)-2-oxoethyl)(nonyl)amino)propylhexanoate, (Z)-methyl8-(2-(dimethylamino)-3-((6-oxo-6-(undec-2-en-1-yloxy)hexyl)oxy)propoxy)octanoate, methyl(9Z)-21-[(dimethylamino)methyl]nonacos-9-enoate, Butyl5-((2-(4-(N-(2-(dinonylamino)ethyl)-N-nonylglycyl)piperazin-1-yl)-2-oxoethyl)(nonyl)amino)pentanoate, (Z)-methyl7-(2-(dimethylamino)-3-((6-oxo-6-(undec-2-en-1-yloxy)hexyl)oxy)propoxy)heptanoate, Propyl6-((2-(4-(N-(2-(dinonylamino)ethyl)-N-nonylglycyl)piperazin-1-yl)-2-oxoethyl)(nonyl)amino)hexanoate, methyl(9Z)-21-[(dimethylamino)methyl]triacont-9-enoate, (Z)-undec-2-en-1-yl6-(2-(dimethylamino)-3-((5-methoxy-5-oxopentyl)oxy)propoxy)hexanoate, methyl(9Z)-19-[(dimethylamino)methyl]pentacos-9-enoate, Ethyl7-((2-(4-(N-(2-(dinonylamino)ethyl)-N-nonylglycyl)piperazin-1-yl)-2-oxoethyl)(nonyl)amino)heptanoate, (Z)-undec-2-en-1-yl6-(2-(dimethylamino)-3-(4-methoxy-4-oxobutoxy)propoxy)hexanoate, methyl6-(2-(dimethylamino)-3-((6-((2-octylcyclopropyl)methoxy)-6-oxohexyl)oxy)propoxy)hexanoate, methyl(9Z)-19-[(dimethylamino)methyl]hexacos-9-enoate, 3-(Dinonylamino)-1-(4-(3-((2-(dinonylamino)ethyl)(nonyl)amino)propanoyl)piperazin-1-yl)propan-1-one, methyl(9Z)-19-[(dimethylamino)methyl]heptacos-9-enoate, 2-((2-(Dinonylamino)ethyl)(nonyl)amino)-1-(4-(ditetradecylglycyl)piperazin-1-yl)ethan-1-one, (Z)-methyl6-(2-(dimethylamino)-3-((6-oxo-6-(undec-2-en-1-yloxy)hexyl)oxy)propoxy)hexanoate, methyl8-(2-(dimethylamino)-3-((8-(2-(6-methoxy-6-oxohexyl)cyclopropyl)octyl)oxy)propoxy)octanoate, methyl8-(2-{9-[(dimethylamino)methyl]octadecyl}cyclopropyl)octanoate, 2-(Dinonylamino)-1-(4-(2-((2-(dinonylamino)ethyl)(nonyl)amino)ethyl)piperidin-1-yl)ethan-1-one, trans-1-methyl-3-[(9Z)-octadec-9-en-1-yloxy]-4-(octyloxy)pyrrolidine, methyl(9Z)-19-{[4-(dimethylamino)butanoyl]oxy}pentacos-9-enoate, methyl(9Z)-19-(dimethylamino)pentacos-9-enoate, (Z)-methy116-(2-(dimethylamino)-3-(nonyloxy)propoxy)hexadec-7-enoate, (2S)-1-[(9Z,12Z)-octadeca-9,12-dien-1-yloxy]decan-2-amine, (12Z,15Z)—N,N-dimethylhenicosa-12,15-dien-4-amine, methyl7-(2-(dimethylamino)-3-((8-(2-(6-methoxy-6-oxohexyl)cyclopropyl)octyl)oxy)propoxy)heptanoate, methyl(9Z)-19-[(dimethylamino)methyl]octacos-9-enoate, 2-((2-(Dinonylamino)ethyl)(nonyl)amino)-1-(4-(2-(dinonylamino)ethyl)piperidin-1-yl)ethan-1-one, Methyl 8-((2-(4-(dinonylglycyl)piperazin-1-yl)-2-oxoethyl)(2-((8-methoxy-8-oxooctyl)(nonyl)amino)ethyl)amino)octanoate, methyl6-(2-(8-(2-(dimethylamino)-3-((5-methoxy-5-oxopentyl)oxy)propoxy)octyl)cyclopropyl)hexanoate, ethyl8-{2-[ll-(dimethylamino)heptadecyl]cyclopropyl}octanoate, Methyl 8-((2-(dinonylamino)ethyl)(2-(4-(dinonylglycyl)piperazin-1-yl)-2-oxoethyl)amino)octanoate, methyl6-(2-(8-(2-(dimethylamino)-3-(4-methoxy-4-oxobutoxy)propoxy)octyl)cyclopropyl)hexanoate, ethyl8-{2-[ll-(dimethylamino)octadecyl]cyclopropyl}octanoate, Methyl 8-((2-((2-(4-(dinonylglycyl)piperazin-1-yl)-2-oxoethyl)(nonyl)amino)ethyl)(nonyl)amino)octanoate, ethyl8-{2-[ll-(dimethylamino)nonadecyl]cyclopropyl}octanoate, (Z)-methy116-(2-(dimethylamino)-3-((8-methoxy-8-oxooctyl)oxy)propoxy)hexadec-7-enoate, Pentyl4-((2-(4-(N-(2-(dinonylamino)ethyl)-N-nonylglycyl)piperazin-1-yl)-2-oxoethyl)(nonyl)amino)butanoate, ethyl8-{2-[ll-(dimethylamino)icosyl]cyclopropyl}octanoate, (Z)-methy116-(2-(dimethylamino)-3-((7-methoxy-7-oxoheptyl)oxy)propoxy)hexadec-7-enoate, Methyl 8-((2-(4-(N-(2-(dinonylamino)ethyl)-N-nonylglycyl)piperazin-1-yl)-2-oxoethyl)(nonyl)amino)octanoate, ethyl8-{2-[9-(dimethylamino)pentadecyl]cyclopropyl}octanoate, (Z)-methy116-(2-(dimethylamino)-3-((5-methoxy-5-oxopentyl)oxy)propoxy)hexadec-7-enoate, (11E,20Z,23Z)—N,N-dimethylnonacosa-11,20,23-trien-10-amine, N,N-dimethyl-1-[(1S,2R)-2-octylcyclopropyl]pentadecan-8-amine, ethyl8-{2-[9-(dimethylamino)hexadecyl]cyclopropyl}octanoate, 2-((2-(Didodecylamino)ethyl)(dodecyl)amino)-1-(5-(dinonylglycyl)-2,5-diazabicyclo[2.2.1]heptan-2-yl)ethan-1-one3, (Z)-methy116-(2-(dimethylamino)-3-(4-methoxy-4-oxobutoxy)propoxy)hexadec-7-enoate, methyl6-(2-(8-(2-(dimethylamino)-3-((6-methoxy-6-oxohexyl)oxy)propoxy)octyl)cyclopropyl)hexanoate, ethyl 8-{2-[9-(dimethylamino)heptadecyl]cyclopropyl}octanoate, 2-(Dinonylamino)-1-(5-(N-(2-(dinonylamino)ethyl)-N-nonylglycyl)-2,5-diazabicyclo[2.2.1]heptan-2-yl)ethan-1-one, 1-[(1S,2R)-2-decylcyclopropyl]-N,N-dimethylpentadecan-6-amine, N1,N1,N2-Tri((9Z,12Z)-octadeca-9,12-dien-1-yl)-N2-(2-(piperazin-1-yl)ethyl)ethane-1,2-diamine, ethyl8-{2-[9-(dimethylamino)octadecyl]cyclopropyl}octanoate, 1-[(1R,2S)-2-heptylcyclopropyl]-N,N-dimethyloctadecan-9-amine, (Z)-methy116-(2-(dimethylamino)-3-((6-methoxy-6-oxohexyl)oxy)propoxy)hexadec-7-enoate, N1,N1,N2-Tri((Z)-octadec-9-en-1-yl)-N2-(2-(piperazin-1-yl)ethyl)ethane-1,2-diamine, N,N-dimethyl-3-{7-[(1S,2R)-2-octylcyclopropyl]heptyl}dodecan-1-amine, methyl8-(2-(dimethylamino)-3-((8-(2-((2-pentylcyclopropyl)methyl)cyclopropyl)octyl)oxy)propoxy)octanoate, ethyl4-{2-[ll-(dimethylamino)icosyl]cyclopropyl}butanoate, trans-1-Methyl-3-[((9Z,12Z)-octadeca-9,12-dienyl)oxy]-4-octyloxy-pyrrolidine, methyl(9Z)-19-(dimethylamino)hexacos-9-enoate, methyl(9Z)-19-{[4-(dimethylamino)butanoyl]oxy}hexacos-9-enoate, (Z)-methy116-(2-(dimethylamino)-3-(heptyloxy)propoxy)hexadec-7-enoate, (2R)-1-[(9Z,12Z)-octadeca-9,12-dien-1-yloxy]dodecan-2-amine, (13Z,16Z)—N,N-dimethyldocosa-13,16-dien-5-amine, N,N-dimethyl-1-[(1R,2S)-2-undecylcyclopropyl]tetradecan-5-amine, methyl7-(2-(dimethylamino)-3-((8-(2-((2-pentylcyclopropyl)methyl)cyclopropyl)octyl)oxy)propoxy)heptanoate, ethyl8-{2-[7-(dimethylamino)hexadecyl]cyclopropyl}octanoate, 2-(Didodecylamino)-N-dodecyl-N-(2-(piperazin-1-yl)ethyl)acetamide, N,N-dimethyl-1-[(1S,2R)-2-octylcyclopropyl]hexadecan-8-amine, N1-(2-(Piperazin-1-yl)ethyl)-N1,N2,N2-tritetradecylethane-1,2-diamine, methyl6-(2-(dimethylamino)-3-((8-(2-((2-pentylcyclopropyl)methyl)cyclopropyl)octyl)oxy)propoxy)hexanoate, ethyl6-{2-[9-(dimethylamino)pentadecyl]cyclopropyl}hexanoate, N,N-dimethyl-1-[(1S,2S)-2-{[(1R,2R)-2-pentylcyclopropyl]methyl}cyclopropyl]nonadecan-10-amine, NN1,N2-Tridodecyl-N2-(2-(piperazin-1-yl)ethyl)ethane-1,2-diamine, methyl5-(2-(dimethylamino)-3-((8-(2-((2-pentylcyclopropyl)methyl)cyclopropyl)octyl)oxy)propoxy)pentanoate, ethyl6-{2-[9-(dimethylamino)hexadecyl]cyclopropyl}hexanoate, N,N-dimethyl-21-[(1S,2R)-2-octylcyclopropyl]henicosan-10-amine, NNN2-Trinonyl-N2-(2-(piperazin-1-yl)ethyl)ethane-1,2-diamine, methyl4-(2-(dimethylamino)-3-((8-(2-((2-pentylcyclopropyl)methyl)cyclopropyl)octyl)oxy)propoxy)butanoate, ethyl6-{2-[9-(dimethylamino)heptadecyl]cyclopropyl}hexanoate, N,N-dimethyl-1-[(1S,2R)-2-octylcyclopropyl]nonadecan-10-amine, N1,N1,N2-Trihexyl-N2-(2-(piperazin-1-yl)ethyl)ethane-1,2-diamine, methyl8-(2-(dimethylamino)-3-((9Z,12Z)-octadeca-9,12-dien-1-yloxy)propoxy)octanoate, ethyl6-{2-[9-(dimethylamino)octadecyl]cyclopropyl}hexanoate, N1-(2-(4-(2-(Didodecylamino)ethyl)piperazin-1-yl)ethyl)-N1,N2,N2-tri((9Z,12Z)-octadeca-9,12-dien-1-yl)ethane-1,2-diamine, methyl7-(2-(dimethylamino)-3-((9Z,12Z)-octadeca-9,12-dien-1-yloxy)propoxy)heptanoate, ethyl(9Z)-21-(dimethylamino)heptacos-9-enoate, 1-[(1S,2R)-2-hexylcyclopropyl]-N,N-dimethylnonadecan-10-amine, 1-methy118-[(2Z)-non-2-en-1-yl]9-{[4-(dimethylamino)butanoyl]oxy}octadecanedioate, N1-(2-(4-(2-(Didodecylamino)ethyl)piperazin-1-yl)ethyl)-N1,N2,N2-tri((Z)-octadec-9-en-1-yl)ethane-1,2-diamine, N,N-dimethyl-1-[(1S,2R)-2-octylcyclopropyl]heptadecan-8-amine, methyl6-(2-(dimethylamino)-3-((9Z,12Z)-octadeca-9,12-dien-1-yloxy)propoxy)hexanoate, ethyl(9Z)-21-(dimethylamino)octacos-9-enoate, dimethyl(9Z)-19-{[4-(dimethylamino)butanoyl]oxy}heptacos-9-enedioate, N1-(2-(4-(2-(Ditetradecylamino)ethyl)piperazin-1-yl)ethyl)-N1,N2,N2-tritetradecylethane-1,2-diamine, methyl5-(2-(dimethylamino)-3-((9Z,12Z)-octadeca-9,12-dien-1-yloxy)propoxy)pentanoate, ethyl8-{[4-(dimethylamino)butanoyl]oxy}-15-(2-octylcyclopropyl)pentadecanoate, ethyl(9Z)-21-(dimethylamino)nonacos-9-enoate, (13Z,16Z)—N,N-dimethyl-3-nonyldocosa-13,16-dien-1-amine, N1-(2-(4-(2-(Didodecylamino)ethyl)piperazin-1-yl)ethyl)-N1,N2,N2-tritetradecylethane-1,2-diamine, methyl9-{[4-(dimethylamino)butanoyl]oxy}-16-(2-octylcyclopropyl)hexadecanoate, methyl4-(2-(dimethylamino)-3-((9Z,12Z)-octadeca-9,12-dien-1-yloxy)propoxy)butanoate, ethyl(9Z)-21-(dimethylamino)triacont-9-enoate, (12Z,15Z)—N,N-dimethyl-2-nonylhenicosa-12,15-dien-1-amine, methyl8-(2-(dimethylamino)-3-((8-(2-octylcyclopropyl)octyl)oxy)propoxy)octanoate, ethyl(9Z)-19-(dimethylamino)pentacos-9-enoate, ethyl(18Z,21Z)-8-{[4-(dimethylamino)butanoyl]oxy}heptacosa-18,21-dienoate, (16Z)—N,N-dimethylpentacos-16-en-8-amine, methyl(9Z)-19-{[4-(dimethylamino)butanoyl]oxy}heptacos-9-enoate, methyl(9Z)-19-(dimethylamino)heptacos-9-enoate, 2-(Didodecylamino)-1-(4-(2-((2-(didodecylamino)ethyl)(dodecyl)amino)ethyl)piperazin-1-yl)ethan-1-one, (Z)-methy116-(2-(dimethylamino)-3-(hexyloxy)propoxy)hexadec-7-enoate, (2S)-1-[(9Z,12Z)-octadeca-9,12-dien-1-yloxy]dodecan-2-amine, methyl 7-(2-(dimethylamino)-3-((8-(2-octylcyclopropyl)-octyl)oxy)propoxy)heptanoate, methyl(19Z,22Z)-9-{[4-(dimethylamino)-butanoyl]oxy} octacosa-19,22-dienoate, ethyl(9Z)-19-(dimethylamino)hexacos-9-enoate, (22Z)—N,N-dimethylhentriacont-22-en-10-amine, methyl5-(2-(dimethylamino)-3-((8-(2-octylcyclopropyl)octyl)oxy)propoxy)pentanoate, ethyl(9Z)-19-(dimethylamino)heptacos-9-enoate, (2-butylcyclopropyl)methy112-{[4-(dimethylamino)butanoyl]oxy}henicosanoate, (20Z)—N,N-dimethylnonacos-20-en-10-amine, N1,N1,N2-Tridodecyl-N2-(2-(4-(2-(dodecyl((9Z,12Z)-octadeca-9,12-dien-1-yl)amino)ethyl)piperazin-1-yl)ethyl)ethane-1,2-diamine, methyl4-(2-(dimethylamino)-3-((8-(2-octylcyclopropyl)octyl)oxy)propoxy)butanoate, ethyl(9Z)-19-(dimethylamino)octacos-9-enoate, (2-octylcyclopropyl)methyl8-{[4-(dimethylamino)butanoyl]oxy}heptadecanoate, (24Z)—N,N-dimethyltritriacont-24-en-10-amine, N1-(2-(4-(2-(Ditetradecylamino)ethyl)piperazin-1-yl)ethyl)-N1,N2,N2-tridodecylethane-1,2-diamine, ethyl(5Z)-17-(dimethylamino)hexacos-5-enoate, (Z)-methyl8-(2-(dimethylamino)-3-(octadec-9-en-1-yloxy)propoxy)octanoate, (2Z)-hept-2-en-1-yl 12-{[4-(dimethylamino)butanoyl]oxy} hemicosanoate, (17Z)—N,N-dimethylnonacos-17-en-10-amine, N1-(2-(4-(2-(Di((Z)-dodec-6-en-1-yl)amino)ethyl)piperazin-1-yl)ethyl)-N1,N2,N2-tridodecylethane-1,2,-diamine, ethyl(9Z)-17-(dimethylamino)hexacos-9-enoate, (Z)-methyl7-(2-(dimethylamino)-3-(octadec-9-en-1-yloxy)propoxy)heptanoate, (2Z)-undec-2-en-1-yl8-{[4-(dimethylamino)butanoyl]oxy}heptadecanoate, (14Z)—N,N-dimethylnonacos-14-en-10-amine, ethyl(7Z)-17-(dimethylamino)tricos-7-enoate, (Z)-methyl5-(2-(dimethylamino)-3-(octadec-9-en-1-yloxy)propoxy)pentanoate, (2-hexylcyclopropyl)methy110-{[4-(dimethylamino)butanoyl]oxy}nonadecanoate, (15Z)—N,N-dimethylheptacos-15-en-10-amine, ethyl(7Z)-17-(dimethylamino)tetracos-7-enoate, (Z)-methyl4-(2-(dimethylamino)-3-(octadec-9-en-1-yloxy)propoxy)butanoate, (2Z)-non-2-en-1-yl10-{[4-(dimethylamino)butanoyl]oxy}nonadecanoate, (20Z)—N,N-dimethylheptacos-20-en-10-amine, N1-(2-(4-(2-(Dioctylamino)ethyl)piperazin-1-yl)ethyl)-N1,N2^{A}V2-tridodecylethane-1,2-diamine, methyl6-(2-(dimethylamino)-3-((8-(2-octylcyclopropyl)octyl)oxy)propoxy)hexanoate, ethyl6-[2-(9-{[4-(dimethylamino)butanoyl]oxy}octadecyl)cyclopropyl]hexanoate, ethyl(7Z)-17-(dimethylamino)pentacos-7-enoate, 1-[(11Z,14Z)-1-nonylicosa-11,14-dien-1-yl]pyrrolidine, ethyl(7Z)-17-(dimethylamino)hexacos-7-enoate, (20Z,23Z)—N-ethyl-N-methylnonacosa-20,23-dien-10-amine, N,N-dimethylheptacosan-10-amine, (2-octylcyclopropyl)methyl6-(3-(decyloxy)-2-(dimethylamino)propoxy)hexanoate, methyl 8-{2-[9-(dimethylamino)octadecyl]cyclopropyl}octanoate, methyl 8-[2-(9-{[4-(dimethylamino)butanoyl]oxy}octadecyl)cyclopropyl]octanoate, methyl 7-(2-(8-(2-(dimethylamino)-3-(octyloxy)propoxy)octyl)cyclopropyl)heptanoate, Heptadecan-9-yl8-((2-hydroxyethyl)(tetradecyl)amino)octanoateRepresentative, 2-((2-(Didodecylamino)ethyl)(dodecyl)amino)-1-(4-(2-(didodecylamino)ethyl)piperazin-1-yl)ethan-1-one, (2S)-1-[(9Z,12Z)-octadeca-9,12-dien-1-yloxy]undecan-2-amine, (17Z,20Z)—N,N-dimemylhexacosa-17,20-dien-9-amine, (18Z)-heptacos-18-en-10-yl4-(dimethylamino)butanoate, (2S)-1-({6-[3B))-cholest-5-en-3-yloxy]hexyl}oxy)-N,N-dimethyl-3-[(9Z)-octadec-9-en-1-yloxy]propan-2-amine, methyl10-{2-[7-(dimethylamino)hexadecyl]cyclopropyl}decanoate, methyl 10-[2-(7-{[4-(dimethylamino)butanoyl]oxy}hexadecyl)cyclopropyl]decanoate, (2S)—N,N-dimethyl-1-({8-[(1R,2R)-2-{[(1S,2S)-2-pentylcyclopropyl]methyl}cyclopropyl]octyl}oxy)tridecan-2-amine, (2-octylcyclopropyl)methyl6-(2-(dimethylamino)-3-(nonyloxy)propoxy)hexanoate, (19Z,22Z)—N,N-dimethyloctacosa-19,22-dien-7-amine, 4-((N-(2-(Dinonylamino)ethyl)-N-nonylglycyl)oxy)pentan-2-yldinonylglycinate, 3-Hydroxybutan-2-ylN-(2-(dinonylamino)ethyl)-N-nonyl, Di(heptadecan-9-yl)8,8′-(26,28-dimethyl-ll,24,30,43-tetraoxo-10,25,29,44-tetraoxa-19,35-diazatripentacontane-19,35-diyl)dioctanoate, Di(heptadecan-9-yl)8,8′-(26,27-dimethyl-ll,24,29,42-tetraoxo-10,25,28,43-tetraoxa-19,34-diazadopentacontane-19,34-diyl)dioctanoate, Di(heptadecan-9-yl) 8,8′-(11,24,29,42-tetraoxo-10,25,28,43-tetraoxa-19,34-diazadopentacontane-19,34-diyl)dioctanoate, Di(heptadecan-9-yl)8,8′-((piperazine-1,4-diylbis(5-oxopentane-5,l-diyl))bis((8-(nonyloxy)-8-oxooctyl)azanediyl))dioctanoate, Di(heptadecan-9-yl)15,18-dimethyl-9,24-bis(8-(nonyloxy)-8-oxooctyl)-14,19-dioxo-9,15,18,24-tetraazadotriacontanedioate, Di(heptadecan-9-yl)15,19-dimethyl-9,25-bis(8-(nonyloxy)-8-oxooctyl)-14,20-dioxo-9,15,19,25-tetraazatritriacontanedioate, Di(heptadecan-9-yl)15,18-diethyl-9,24-bis(8-(nonyloxy)-8-oxooctyl)-14,19-dioxo-9,15,18,24-tetraazadotriacontanedioate, N,N-dimethyl-3-{[(9Z,12Z)-octadeca-9,12-dien-1-yloxy]methyl}dodecan-1-amine, methyl8-[2-(ll-{[4-(dimethylamino)butanoyl]oxy}octadecyl)cyclopropyl]octanoate, methyl8-{2-[ll-(dimethylamino)heptadecyl]cyclopropyl}octanoate(Compound18), Heptadecan-9-yl8-((2-hydroxyethyl)(8-(nonyloxy)-8-oxooctyl)amino)octanoate, (2-octylcyclopropyl)methyl6-(2-(dimethylamino)-3-(heptyloxy)propoxy)hexanoate, (17Z)—N,N-dimethylhexacos-17-en-9-amine, N,N-dimethyl-2-{[(9Z,12Z)-octadeca-9,12-dien-1-yloxy]methyl}undecan-1-amine, methyl8-{2-[ll-(dimethylamino)octadecyl]cyclopropyl}octanoate, (2-octylcyclopropyl)methyl6-(2-(dimethylamino)-3-(hexyloxy)propoxy)hexanoate, (18Z)—N,N-dimethylheptacos-18-en-10-amine, 2-((2-(Dinonylamino)ethyl)(nonyl)amino)ethyltetradecanoate, 2-((2-(Dinonylamino)ethyl)(nonyl)amino)ethylnonanoate, TetradecylN-(2-(dinonylamino)ethyl)-N-nonylglycinate, NonylN-(2-(dinonylamino)ethyl)-N-nonylglycinate, 4-(2-((2-(dinonylamino)ethyl)(nonyl)amino)acetamido)butylpentanoate, 1,1′-(Piperazine-1,4-diyl)bis(5-(didecylamino)pentan-1-one, 2-((2-(dinonylamino)ethyl)(nonyl)armno)-N-tetradecylacetamide, N-decyl-2-((2-(dinonylamino)ethyl)(nonyl)amino), N1-(3-(3-(dinonylamino)propoxy)propyl)-N1,N2,N2-trinonylethane-1,2-diamine, N1-(2-(dinonylamino)ethyl)-NN8,N8-trinonyloctane-1,8-diamine, methyl8-[2-(ll-{[4-(dimethylamino)butanoyl]oxy}nonadecyl)cyclopropyl]octanoate, methyl8-{2-[ll-(dimethylamino)nonadecyl]cyclopropyl}octanoate, (Z)-undec-2-en-1-yl6-(3-(decyloxy)-2-(dimethylamino)propoxy)hexanoate, (2R,12Z,15Z)—N,N-dimethyl-1-(undecyloxy)henicosa-12,15-dien-2-amine, (21Z,24Z)—N,N-dimethyltriaconta-21,24-dien-9-amine, 2-(dinonylamino)-N-(4-(2-((2-(dinonylamino)ethyl)(nonyl)amino)-N-methylacetamido)butyl)-N-methylacetamide, 7,10-dimethyl-13,16-dinonyl-6,11-dioxo-4-tetradecyl-4,7,10,13,16-pentaazapentacosyldecanoate, 2-(dinonylamino)-N-(2-(2-((2-(dinonylamino)ethyl)(nonyl)amino)-N-ethylacetamido)ethyl)-N-ethylacetamide, 2-(dinonylamino)-N-(3-(2-((2-(dinonylamino)ethyl)(nonyl)amino)-N-methylacetamido)propyl)-N-methylacetamide, 2-((2-(di((Z)-non-3-en-1-yl)amino)ethyl)((Z)-non-3-en-1-yl)amino)-N-(2-(2-(dinonylamino)-N-methylacetamido)ethyl)-N-methylacetamide, 2-(dinonylamino)-N-(2-(2-((2-(dinonylamino)ethyl)(nonyl)amino)acetamido)ethyl)acetamide, Pentyl 8,11-dimethyl-5,14,17-trinonyl-7,12-dioxo-5,8,11,14,17-pentaazahexacosanoate2-((2-(Dinonylamino)ethyl)(nonyl)aniino)-N-methyl-N-(2-(methylandno)ethyl)acetami, 2-(Dinonylamino)-N-(2-(2-((2-(dinonylamino)ethyl)(nonyl)amino)-N-methylacetamido)ethyl)-N-methylacetamide2-(Dinonylamino)-N-methyl-N-(2-(methylamino)ethyl)acetamide, 2-((N-(2-(Dinonylamino)ethyl)-N-nonylglycyl)oxy)ethyldinonylglycinate2-Hydroxyethyldinonylglycinate, (Z)-undec-2-en-1-yl 6-(2-(dimethylamino)-3-(nonyloxy)propoxy)hexanoate, (2R,12Z,15Z)-1-(hexadecyloxy)-N,N-dimethylhenicosa-12,15-dien-2-amine, (22Z,25Z)—N,N-dimethylhentriaconta-22,25-dien-10-amine, 1,1-(Piperazine-1,4-diyl)bis(4-(didecylamino)butan-1-one)fert-Butyl4-(didecylaminobutanoate, Heptyl5-(4-(N-(2-(dinonylamino)ethyl)-N-nonylglycyl)piperazin-1-yl)-5-oxopentanoate5-(Heptloxy)-5-oxopentanoicacid, Heptyl5-(4-(N-(2-(dinonylamino)ethyl)-N-nonylglycyl)piperazin-1-yl)-5-oxopentanoate5-(Heptloxy)-5-oxopentanoic, (Z)-4-((2-(4-(N-(2-(dinonylamino)ethyl)-N-nonylglycyl)piperazin-1-yl)-2-oxoethyl)(tetradecyl)amino)but-2-en-1-ylnonanoate(Z)-4-Hydroxybut-2-en-1-ylnonanoate, (Z)-3-((2-(4-(N-(2-(Dinonylamino)ethyl)-N-nonylglycyl)piperazin-1-yl)-2-oxoethyl)(tetradec-9-en-1-yl)amino)propy 1 decanoate(Z)-Tetradec-9-en-1-ylmethanesulfonate, methyl8-[2-(9-{[4-(dimethylamino)butanoyl]oxy}pentadecyl)cyclopropyl]octanoate, methyl8-{2-[9-(dimethylamino)pentadecyl]cyclopropyl}octanoate, (Z)-undec-2-en-1-yl6-(2-(dimethylamino)-3-(heptyloxy)propoxy)hexanoate, (2R,12Z,15Z)-1-(hexyloxy)-N,N-dimethylhenicosa-12,15-dien-2-amine, (16Z,19Z)—N,N-dimethylpentacosa-16,19-dien-6-amine, Methyl8-((2-(4-(N-(2-(Di((Z)-non-3-en-1-yl)amino)ethyl)-N—((Z)-non-3-en-1-yl)glycyl)piperazin-1-yl)-2-oxoethyl)(nonyl)amino)octanoatefert-Butyl4-(nonylglycyl)piperazine-1-carboxylate, 3-((2-(4-(N-(2-(Dinonylamino)ethyl)-N-nonylglycyl)piperazin-1-yl)-2-oxoethyl)(tetradecyl)amino)propyl(Z)-dec-3-enoate(Z)-Dec-3-en-1-ol, 2-((2-(Di((Z)-non-3-en-1-yl)amino)ethyl)((Z)-non-3-en-1-yl)amino)-1-(4-(dinonylglycyl)piperazin-1-yl)ethan-1-one(Z)-1-Bromonon-4-ene, 3-((2-(4-(N-(2-(Dinonylamino)ethyl)-N-nonylglycyl)piperazin-oxoethyl)(dodecyl)amino)propyloctanoatetot-Butyldodecylglycinate, S-Pentyl4-((2-(4-(N-(2-(dinonylamino)ethyl)-N-nonylglycyl)piperazin-1-yl)-2-oxoethyl)(nonyl)amino)butanethioate, 3-((2-(1-(N-(2-(Dinonylamino)ethyl)-N-nonylglycyl)piperidin-yl)ethyl)(nonyl)amino)propyl3-methylhexanoatefert-Butyl4-(2-((3-((3-methylhexanoyl)oxy)propyl)(nonyl)amino)ethyl)piperidine-1-, 3-((2-(1-(N-(2-(Dinonylamino)ethyl)-N-nonylglycyl)piperidin-4-yl)ethyl)(nonyl)amino)-2-methylpropylhexanoate, 3-((2-(4-(N-(2-(Dinonylamino)ethyl)-N-nonylglycyl)piperazin-oxoethyl)(nonyl)amino)propyl3-methylhexanoate, 3-((2-(4-(N-(2-(Dinonylamino)ethyl)-N-nonylglycyl)piperazin-oxoethyl)(nonyl)amino)-2-methylpropylhexanoate, methyl8-[2-(9-{[4-(dimethylamino)butanoyl]oxy}hexadecyl)cyclopropyl]octanoate, methyl8-{2-[9-(dimethylamino)hexadecyl]cyclopropyl}octanoate, (Z)-undec-2-en-1-yl6-(2-(dimethylamino)-3-(hexyloxy)propoxy)hexanoate, (2R,12Z,15Z)-1-(decyloxy)-N,N-dimethylhenicosa-12,15-dien-2-amine, (17Z,20Z)—N,N-dimethylhexacosa-17,20-dien-7-amine, 2-((2-(Dinonylamino)ethyl)(nonyl)amino)ethyl1-(dinonylglycyl)piperidine-4-carboxylate, l-(2-(Dinonylamino)ethyl)4-(2-((2-(dinonylamino)ethyl)(nonyl)amino)ethyl)cyclohexane-1,4-dicarboxylate, 2-(Dinonylamino)ethan-1-ol, Methyl 12-((2-(1-(N-(2-(dinonylamino)ethyl)-N-nonylglycyl)pyrrolidin-3-yl)ethyl)(tetradecyl)amino)dodecanoate, tert-Butyl 3-(2-((12-methoxy-12-oxododecyl)(tetradecyl)amino)ethyl)pyrrolidine-1-carboxylate, Heptyl 6-((2-(1-(N-(2-(dinonylamino)ethyl)-N-nonylglycyl)pyrrolidin-3-yl)ethyl)(tetradecyl)amino)hexanoatetot-Butyl3-(2-((6-(heptyloxy)-6-oxohexyl)(tetradecyl)amino)ethyl)pyrrolidine-1-carboxylate, Pentyl 8-((2-(1-(N-(2-(dinonylamino)ethyl)-N-nonylglycyl)pyrrolidin-3-yl)ethyl)(tetradecyl)amino)octanoate/er/-Butyl3-(2-(tetradecylamino)ethyl)pyrrolidine-1-carboxylate, Methyl 12-((2-(1-(N-(2-(dinonylamino)ethyl)-N-nonylglycyl)piperidin-3-yl)ethyl)(tetradecyl)amino)dodecanoate-Butyl3-(2-((12-methoxy-12-oxododecyl)(tetradecyl)amino)ethyl)piperidine-1-carboxylate, 3-((2-(1-(N-(2-(Dinonylamino)ethyl)-N-nonylglycyl)piperidin-3-yl)ethyl)(tetradecyl)amino)propyldecanoate, Heptyl 6-((2-(1-(N-(2-(dinonylamino)ethyl)-N-nonylglycyl)piperidin-3-yl)ethyl)(tetradecyl)amino)hexanoate, Pentyl 8-((2-(1-(N-(2-(dinonylamino)ethyl)-N-nonylglycyl)piperidin-3-yl)ethyl)(tetradecyl)amino)octanoate, Pentyl6-((2-(4-(2-((2-(didodecylamino)ethyl)(dodecyl)amino)ethyl)piperazin-1-yl)ethyl)(dodecyl)amino)hexanoateStep1:Pentyl6-bromohexanoate, methyl8-[2-(9-{[4-(dimethylamino)butanoyl]oxy}heptadecyl)cyclopropyl]octanoate, methyl8-{2-[9-(dimethylamino)heptadecyl]cyclopropyl}octanoate, (2S,12Z,15Z)—N,N-dimethyl-1-(octyloxy)henicosa-12,15-dien-2-amine, (2-octylcyclopropyl)methyl6-(2-(dimethylamino)-3-(octyloxy)propoxy)hexanoate, (18Z,21Z)—N,N-dimethylheptacosa-18,21-dien-8-amine, trans-1-methyl-3,4-bis(((Z)-hexadec-9-enoyloxy)methyl)pyrrolidine, (Z)—Non-2-en-1-yl4-((2-(4-(N-(2-(dinonylamino)ethyl)-N-nonylglycyl)piperazin-1-yl)-2-oxoethyl)(tetradecyl)amino)butanoate, trans-1-methyl-3,4-bis(((9Z,12Z)-octadeca-9,12-dienoyloxy)methyl)pyrrolidine, Methy112-((2-(4-(N-(2-(dinonylamino)ethyl)-N-nonylglycyl)piperazin-1-yl)-2-oxoethyl)(tetradecyl)amino)dodecanoate, ethyl(7Z)-17-[2-(dimethylamino)ethyl]hexacos-7-enoate, trans-1-methyl-3,4-bis(((Z)-octadeca-9-enoyloxy)methyl)pyrrolidine, Methyl 12-((2-(1-(N-(2-(dinonylamino)ethyl)-N-nonylglycyl)piperidin-4-yl)ethyl)(tetradecyl)amino)dodecanoate, methyl8-(2-{111-;2-(dimethylamino)ethyl]heptadecyl}cyclopropyl)octanoate, 2-(1-(N-(2-(Dinonylamino)ethyl)-N-nonylglycyl)piperidin-4-yl)ethyldinonylglycinatefert-Butyl4-(2-((dinonylglycyl)oxy)ethyl)piperidine-1-carboxylate, 1,-(piperazine-1,4-diyl)bis(2-(dinonylamino)ethan-1-one), methyl8-(2-{9-[2-(dimethylamino)ethyl]pentadecyl}cyclopropyl)octanoate, methyl(7Z)-19-{[4-(dimethylamino)butanoyl]oxy}octacos-7-enoate, methyl(7Z)-19-(dimethylamino)octacos-7-enoate, cis-1-methyl-3-[(9Z,12Z)-octadeca-9,12-dien-1-yloxy]-4-(octyloxy)pyrrolidine, 2-(Didodecylamino)-1-(4-(N-(2-(didodecylamino)ethyl)-N-dodecylglycyl)piperazin-1-yl)ethan-1-one, (Z)-undec-2-en-1-yl6-(2-(dimethylamino)-3-(octyloxy)propoxy)hexanoate, (2SN,N-dimethyl-1-[(9Z,12Z)-octadeca-9,12-dien-1-yloxy]decan-2-amine(Compound11), (19Z,22Z)—N,N-dimeihyloctacosa-19,22-dien-9-amine, methyl8-(2-{9-[2-(dimethylamino)ethyl]hexadecyl}cyclopropyl)octanoate, 5-((2-(4-(N-(2-(Dinonylamino)ethyl)-N-nonylglycyl)piperazin-oxoethyl)(nonyl)amino)pentylmethylcarbonate, methyl8-(2-{9-[2-(dimethylamino)ethyl]heptadecyl}cyclopropyl)octanoate, methyl(7Z)-19-[2-(dimethylamino)ethyl]octacos-7-enoate, (Z)-Pent-2-en-1-yl4-((2-(4-(N-(2-(dinonylamino)ethyl)-N-nonylglycyl)piperazin-1-yl)-2-oxoethyl)(nonyl)amino)butanoate, methyl(1lZ)-19-[2-(dimethylamino)ethyl]octacos-ll-enoate, methyl(9Z)-21-[2-(dimethylamino)ethyl]heptacos-9-enoate, methyl(9Z)-21-[2-(dimethylamino)ethyl]octacos-9-enoate, methyl(9Z)-21-[2-(dimethylamino)ethyl]nonacos-9-enoate, 2-(1-(N-(2-(Dinonylamino)ethyl)-N-nonylglycyl)pyrrolidin-3-yl)ethyldinonylglycinate, methyl(9Z)-21-[2-(dimethylamino)ethyl]triacont-9-enoate, (1-(N-(2-(Dinonylamino)ethyl)-N-nonylglycyl)pyrrolidin-3-yl)methyldinonylglycinate, methyl(9Z)-19-[2-(dimethylamino)ethyl]pentacos-9-enoate, methyl(9Z)-19-[2-(dimethylamino)ethyl]hexacos-9-enoate, methyl6-(2-(8-(3-(decyloxy)-2-(dimethylamino)propoxy)octyl)cyclopropyl)hexanoate, methyl(1lZ)-19-{[4-(dimethylamino)butanoyl]oxy}octacos-ll-enoate, methyl(1lZ)-19-(dimethylamino)octacos-11-enoate, (2S)—N,N-dimethyl-1-[(9Z,12Z)-octadeca-9,12-dien-1-yloxy]dodecan-2-amine, (14Z,17Z)—N,N-dimethyltricosa-14,17-dien-4-amine, Methyldi((9Z,12Z)-octadeca-9,12-dienyl)amine, methyl(9Z)-19-{[4-(dimethylamino)butanoyl]oxy}octacos-9-enoate, methyl(9Z)-19-(dimethylamino)octacos-9-enoate, (Z)-methy117-(2-(dimethylamino)-3-(octyloxy)propoxy)heptadec-8-enoate, (3R,4R)-3,4-bis((Z)-hexadec-9-enyloxy)-1-methylpyrrolidine, (2S)—N,N-dimethyl-1-[(9Z,12Z)-octadeca-9,12-dien-1-yloxy]undecan-2-amine, (20Z,23Z)-nonacosa-20,23-dien-10-yl4-(dimethylamino)butanoate, (20Z,23Z)—N,N-dimethylnonacosa-20,23-dien-10-amine, 3-((6Z,9Z,28Z,31Z)-heptatriaconta-6,9,28,31-tetraen-19-yloxy)-N,N-dimethylpropan-1-amine, 3-((6Z,9Z,28Z,31Z)-heptatriaconta-6,9,28,31-tetraen-19-yloxy)-N,N-dimethylpropan-1-amine, (6Z,9Z,28Z,31Z)-heptatriaconta-6,9,28,31-tetraen-19-yl-4-(dimethylamino)butanoate), (6Z,16Z)-12-((Z)-dec-4-enyl)docosa-6,16-dien-ll-yl5-(dimethylamino)pentanoate, (6Z,16Z)-12-((Z)-dec-4-enyl)docosa-6,16-dien-ll-yl5-(dimethylamino)pentanoat, (6Z,16Z)-12-((Z)-dec-4-enyl)docosa-6,16-dien-11-yl5-(dimethylamino)pentanoate, L-arginine-alpha-(2,3-dilauryloxy)propylamide, L-lysine-alpha-(2,3-dilauryloxy)propylamide, 2,3-dioleyloxypropylamine, 2,3-distearyloxypropylamine, 2,3-dilauryloxypropylamine, dilinoleylmethyl4-(dimethylamino)propylether), dilinoleylmethyl4-(dimethylamino)butylether), and 2,2-dilinoleyl-4-(2-dimethylaminoethyl)-[1,3]-dioxolane.

In some embodiments, the at least one non-cationic lipid comprises at least one phospholipid, at least one fusogenic lipid, at least one anionic lipid, at least one helper lipid, at least one neutral lipid, or any combination thereof. In some embodiments, the LNP may be essentially devoid of the at least one non-cationic lipid. In some embodiments, the LNP may contain no amount of the at least one non-cationic lipid.

In some embodiments, at least one non-cationic lipid may be selected from, but is not limited to, at least one of 1,2-di-O-octadecenyl-sn-glycero-3-phosphocholine (18:0 Diether PC), DSPC but with 3unsaturateddoublebondspertail (18:3 PC), Acylcarnosine (AC), 1-hexadecyl-sn-glycero-3-phosphocholine (C16 Lyso PC), N-oleoyl-sphingomyelin (SPM) (C18:1), N-lignoceryl SPM (C24:0), N-nervonoylshphingomyelin (C24:1), carbamoyl]cholesterol (Cet-P), cholesterol hemisuccinate (CHEMS), cholesterol (Chol), Cholesterol hemidodecanedicarboxylic acid (Chol-C12), 12-Cholesteryloxycarbonylaminododecanoic acid (Chol-C13N), Cholesterol hemioxalate (Chol-C2), Cholesterol hemimalonate (Chol-C3), N-(Cholesteryl-oxycarbonyl)glycine (Chol-C3N), Cholesterol hemiglutarate (Chol-C5), Cholesterol hemiadipate (Chol-C6), Cholesterol hemipimelate (Chol-C7), Cholesterol hemisuberate (Chol-C8), Cardiolipin (CL), 1,2-bis(tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis-n-heptadecanoyl phosphatidylcholine (DHPC), dihexadecoyl-phosphoethanolamine (DUPE), 1,2-dilinoleoyl-sn-glycero-3-phosphocholine (DLPC), 1,2-dilauroyl-sn-glycero-3-PE (DLPE), Dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-dioleoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-letrahydro-pyran-2-ylsulfanyl)-propionamide (DOGP4aMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylcholine (DPPC), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoylphosphoethanolamineimidazole (DSPEI), 1,2-diundecanoyl-sn-glycero-phosphocholine (DUPC), egg phosphatidylcholine (EPC), N-histidinylcholesterolcarbamate (HCChol), histaminedistearoylglycerol (HDSG), N-histidinylcholesterol hemisuccinate (HistChol), 1,2-Dipalmitoylglycerol-hemisuccinate-Na-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-a-histidinyl-Na-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-maleimidomethyl)cyclohexane-carboxamide] (MCC-PE), 1,2-diphytanoyl-sn-glycero-3-phosphoethanolamine (ME 16.0 PE), 1-myristoyl-2-hydroxy-sn-glycero-phosphocholine (MHPC), a thiol-reactive maleimide headgroup lipid e.g.1,2-dioleoyl-sn-glycero-3-phosphoethanolamine-N-[4-(p-maleimidophenyl)but-yramid (MPB-PE), Nervonic Acid (NA), sodium cholate (NaChol), 1,2-dioleoyl-sn-glycero-3-[phosphoethanolamine-N-dodecanoyl (NC12-DOPE), OleicAcid (OA), 1-oleoyl-2-cholesteryl hemisuccinoyl-sn-glycero-3-phosphocholine (OChemsPC), phosphatidicacid (PA), phosphatidylethanolamine lipid (PE), PE lipid conjugated with polyethylene glycol(PEG) (e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissamine rhodamineB-phosphatidylethanolamine lipid (Rh-PE), purifiedsoy-derivedmixtureofphospholipids (SIOO), phosphatidylcholine (SM), 18-1-trans-PE,1-stearoyl-2-oleoyl-phosphatidyethanolamine (SOPE), soybean phosphatidylcholine (SPC), sphingomyelins (SPM), alpha,alpha-trehalose-6,6′-dibehenate (TDB), 1,2-dielaidoyl-sn-glycero-3-phophoethanolamine (transDOPE), ((23S,5R)-3-(bis(hexadecyloxy)methoxy)-5-(5-methyl-2,4-dioxo-3,4-dihydropyrimidin-1(2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE,16-O-dimethyl PE, and dioleylphosphatidylethanolamine.

In some embodiments, the LNP comprises an ionizable lipid or lipid-like material. As a non-limiting example, the ionizable lipid may be C12-200, CKK-E12, 5A2-SC8, BAMEA-016B, or 7C1. Other ionizable lipids are known in the art and are useful herein.

In some embodiments, the LNP comprises a phospholipid. As a non-limiting example, the phospholipid (helper) may be DOPE, DSPC, DOTAP, or DOTMA.

In some embodiments, the LNP comprises a PEG derivative. As a non-limiting example, the PEG derivative may be a lipid-anchored such as PEG is C14-PEG2000, C14-PEG1000, C14-PEG3000, C14-PEG5000, C12-PEG1000, C12-PEG2000, C12-PEG3000, C12-PEG5000, C16-PEG1000, C16-PEG2000, C16-PEG3000, C16-PEG5000, C18-PEG1000, C18-PEG2000, C18-PEG3000, or C18-PEG5000.

In some embodiments, the at least one sterol comprises at least one cholesterol or cholesterol derivative. In some embodiments, the LNP may be essentially devoid of an at least one sterol. In some embodiments, the LNP may contain no amount of the at least one sterol.

In some embodiments, the at least one particle-activity-modifying-agent comprises at least one component that reduced aggregation of particles, at least one component that decreases clearing of the LNP from circulation in a subject, at least component that increases the LNP's ability to traverse mucus layers, at least one component that decreases a subjects immune response to administration of the LNP, at least one component that modifies membrane fluidity of the LNP, at least one component that contributes to the stability of the LNP, or any combination thereof. In some embodiments, the LNP may be essentially devoid of the at least one particle-activity-modifying-agent. In some embodiments, the LNP may contain no amount of the at least one particle-activity-modifying-agent.

In some embodiments, the particle-activity-modifying-agent may be comprised of a polymer. In some embodiments, the polymer comprising the particle-activity-modifying-agent may be comprised of at least one polyethylene glycol (PEG), at least one polypropylene glycol (PPG), poly(2-oxazoline) (POZ), at least one polyamide (ATTA), at least one cationic polymer, or any combination thereof.

In some embodiments, the average molecular weight of the polymer moiety (e.g., PEG) may be between 500 and 20,000 Daltons. In some embodiments, the molecular weight of the polymer may be about 500 to 20,000 Daltons, e.g., about 500 to 2,000, about 2,000 to 6,000, about 6,000 to 10,000, about 10,000 to 14,000, about 14,000 to 18,000, or about 18,000 to 20,000 Daltons.

In some embodiments the polymer (e.g., PEG) is conjugated to at least one lipid. In some embodiments the lipid conjugated to the polymer comprised of at least one neutral lipid, at least one phospholipid, at least one anionic lipid, at least one cationic lipid, at least one cholesterol, at least one cholesterol derivative, or any combination thereof.

In some embodiments, the lipid conjugated to the polymer may be selected from, but is not limited to, at least one of the cationic, non-cationic, or sterol lipids listed previously.

In some embodiments, the at least one PEG-lipid conjugate may be selected from, but is not limited to at least one of Siglec-1L-PEG-DSPE, R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N(Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, CI8PEG5000, CI8PEG3000, CI8PEG2000, CI6PEG2000, CI4PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X.

The amounts and ratios of LNP components may be varied by any amount dependent on the desired form, structure, function, cargo, target, or any combination thereof. The amount of each component may be expressed in various embodiments as percent of the total molar mass of all lipid or lipid conjugated components accounted for by the indicated component (mol %), The amount of each component may be expressed in various embodiments as the relative ratio of each component based on molar mass (Molar Ratio). The amount of each component may be expressed in various embodiments as the weight of each component used to formulate the LNP prior to fabrication (mg or equivalent). The amount of each component may be expressed in various embodiments by any other method known in the art. Any formulation given in one representation of component amounts (“units”) is expressly meant to encompass any formulation expressed in different units of component amounts, wherein those representations are effectively equivalent when converted into the same units. In some embodiments, “effectively equivalent” means two or more values within about 10% of one another.

In some embodiments, the LNP comprises at least one cationic lipid in an amount of about 0.1 to 100 mol %. In some embodiments, the LNP comprises at least one cationic lipid in an amount of about 20 to 60 mol %. In some embodiments, the LNP comprises at least one cationic lipid in an amount of about 50 to 85 mol %. In some embodiments, the LNP comprises at least one cationic lipid in an amount of less than about 20 mol %. In some embodiments, the LNP comprises at least one cationic lipid in an amount of more than about 60 mol % or about 85 mol %. In some embodiments, the LNP comprises at least one cationic lipid in an amount of about 95 mol % or less. In some embodiments, the LNP comprises a cationic lipid in an amount of less than or equal to about 95, 90, 85, 80, 75, 70, 65, 60, 55, 50, 45, 40, 35, 30, 25, 20, 15, 10, and 5 mol %. In some embodiments, the LNP comprises at least one cationic lipid in an amount of more than or equal to about 5, 10, 15, 20, 25, 30, 35, 40, 45, 50, 55, 60, 65, 70, 75, 80, 85, 90, and 95 mol %. In some embodiments, the LNP comprises at least one cationic lipid in an amount from about 20 to 30 mol %, 20 to 35 mol %, 20 to 40 mol %, 20 to 45 mol %, 20 to 50 mol %, 20 to 55 mol %, 20 to 60 mol %, 20 to 65 mol %, 20 to 70 mol %, 20 to 75 mol %, 20 to 80 mol %, 20 to 85 mol %, 20 to 90 mol %, 25 to 35 mol %, 25 to 40 mol %, 25 to 45 mol %, 25 to 50 mol %, 25 to 55 mol %, 25 to 60 mol %, 25 to 65 mol %, 25 to 70 mol %, 25 to 75 mol %, 25 to 80 mol %, 25 to 85 mol %, 25 to 90 mol %, 30 to 40 mol %, 30 to 45 mol %, 30 to 50 mol %, 30 to 55 mol %, 30 to 60 mol %, 30 to 65 mol %, 30 to 70 mol %, 30 to 75 mol %, 30 to 80 mol %, 30 to 85 mol %, 30 to 90 mol %, 35 to 40 mol %, 35 to 45 mol %, 35 to 50 mol %, 35 to 55 mol %, 35 to 60 mol %, 35 to 65 mol %, 35 to 70 mol %, 35 to 75 mol %, 35 to 80 mol %, 35 to 85 mol %, 35 to 90 mol %, 40 to 45 mol %, 40 to 50 mol %, 40 to 55 mol %, 40 to 60 mol %, 40 to 65 mol %, 40 to 70 mol %, 40 to 75 mol %, 40 to 80 mol %, 40 to 85 mol %, 40 to 90 mol %, 45 to 55 mol %, 45 to 60 mol %, 45 to 65 mol %, 45 to 70 mol %, 45 to 75 mol %, 45 to 80 mol %, 45 to 85 mol %, 45 to 90 mol %, 50 to 60 mol %, 50 to 65 mol %, 50 to 70 mol %, 50 to 75 mol %, 50 to 80 mol %, 50 to 85 mol %, 50 to 90 mol %, 55 to 65 mol %, 55 to 70 mol %, 55 to 75 mol %, 55 to 80 mol %, 55 to 85 mol %, 55 to 90 mol %, 60 to 70 mol %, 60 to 75 mol %, 60 to 80 mol %, 60 to 85 mol %, 60 to 90 mol %, 65 to 75 mol %, 65 to 80 mol %, 65 to 85 mol %, 65 to 90 mol %, 70 to 80 mol %, 70 to 85 mol %, 70 to 90 mol %, 75 to 85 mol %, 75 to 90 mol %, 80 to 90 mol % or 85 to 95 mol %.

In some embodiments, the LNP comprises at least one non-cationic lipid in an amount of about 0.1 to 100 mol %. In some embodiments, the LNP comprises at least one non-one cationic lipid in an amount of about 5 to 35 mol %. In some embodiments, the LNP comprises at least one cationic lipid in an amount of about 5 to 25 mol %. In some embodiments, the LNP comprises at least one non-cationic lipid in an amount of less than about 5 mol %. In some embodiments, the LNP comprises at least one non-cationic lipid in an amount of more than about 25 mol % or about 35 mol %. In some embodiments, the LNP comprises at least one non-cationic lipid in an amount of about 95 mol % or less. In some embodiments, the LNP comprises at least one non-cationic lipid in an amount of less than or equal to about 95, 90, 85, 80, 75, 70, 65, 60, 55, 50, 45, 40, 35, 30, 25, 20, 15, 10, and 5 mol %. In some embodiments, the LNP comprises at least one non-cationic lipid in an amount of more than or equal to about 5, 10, 15, 20, 25, 30, 35, 40, 45, 50, 55, 60, 65, 70, 75, 80, 85, 90, and 95 mol %. In some embodiments, the LNP comprises at least one non-cationic lipid in an amount from about 5 to 15 mol %, 5 to 25 mol %, 5 to 35 mol %, 5 to 45 mol %, 5 to 55 mol %, 10 to 20 mol %, 10 to 30 mol %, 10 to 40 mol %, 10 to 50 mol %, 15 to 25 mol %, 15 to 35 mol %, 15 to 45 mol %, 20 to 30 mol %, 20 to 40 mol %, 20 to 50 mol %, 25 to 35 mol %, 25 to 45 mol %, 30 to 40 mol %, 30 to 50 mol %, and 35 to 45 mol %.

In some embodiments, the LNP comprises at least one sterol in an amount of about 0.1 to 100 mol %. In some embodiments, the LNP comprises at least one sterol in an amount of about 20 to 45 mol %. In some embodiments, the LNP comprises at least one sterol in an amount of about 25 to 55 mol %. In some embodiments, the LNP comprises at least one sterol in an amount of less than about 20 mol %. In some embodiments, the LNP comprises at least one sterol in an amount of more than about 45 mol % or about 55 mol %. In some embodiments, the LNP comprises at least one sterol in an amount of about 95 mol % or less. In some embodiments, the LNP comprises at least one sterol in an amount of less than or equal to about 95, 90, 85, 80, 75, 70, 65, 60, 55, 50, 45, 40, 35, 30, 25, 20, 15, 10, and 5 mol %. In some embodiments, the LNP comprises at least one sterol in an amount of more than or equal to about 5, 10, 15, 20, 25, 30, 35, 40, 45, 50, 55, 60, 65, 70, 75, 80, 85, 90, and 95 mol %. In some embodiments, the LNP comprises at least one sterol in an amount from about 10 to 20 mol %, 10 to 30 mol %, 10 to 40 mol %, 10 to 50 mol %, 10 to 60 mol %, 15 to 25 mol %, 15 to 35 mol %, 15 to 45 mol %, 15 to 55 mol %, 15 to 65 mol %, 20 to 30 mol %, 20 to 40 mol %, 20 to 50 mol %, 20 to 60 mol %, 25 to 35 mol %, 25 to 45 mol %, 25 to 55 mol %, 25 to 65 mol %, 30 to 40 mol %, 30 to 50 mol %, 30 to 60 mol %, 35 to 45 mol %, 35 to 55 mol %, 35 to 65 mol %, 40 to 50 mol %, 40 to 60 mol %, 45 to 55 mol %, 45 to 65 mol %, 50 to 60 mol %, and 55 to 65 mol %.

In some embodiments, the LNP comprises at least one particle-activity-modifying-agent in an amount of about 0.1 to 100 mol %. In some embodiments, the LNP comprises at least one particle-activity-modifying-agent in an amount of about 0.5 to 15 mol %. In some embodiments, the LNP comprises at least one particle-activity-modifying-agent in an amount of about 15 to 40 mol %. In some embodiments, the LNP comprises at least one particle-activity-modifying-agent in an amount of less than about 0.1 mol %. In some embodiments, the LNP comprises at least one particle-activity-modifying-agent in an amount of more than about 15 mol % or about 40 mol %. In some embodiments, the LNP comprises at least one particle-activity-modifying-agent in an amount of about 95 mol % or less. In some embodiments, the LNP comprises at least one particle-activity-modifying-agent in an amount of less than or equal to about 95, 90, 85, 80, 75, 70, 65, 60, 55, 50, 45, 40, 35, 30, 25, 20, 15, 10, and 5 mol %. In some embodiments, the LNP comprises at least one particle-activity-modifying-agent in an amount of more than or equal to about 5, 10, 15, 20, 25, 30, 35, 40, 45, 50, 55, 60, 65, 70, 75, 80, 85, 90, and 95 mol %. In some embodiments, the LNP comprises at least one particle-activity-modifying-agent in an amount from about 0.1 to 1 mol %, 0.1 to 2 mol %, 0.1 to 3 mol %, 0.1 to 4 mol %, 0.1 to 5 mol %, 0.1 to 6 mol %, 0.1 to 7 mol %, 0.1 to 8 mol %, 0.1 to 9 mol %, 0.1 to 10 mol %, 0.1 to 15 mol %, 0.1 to 20 mol %, 0.1 to 25 mol %, 1 to 2 mol %, 1 to 3 mol %, 1 to 4 mol %, 1 to 5 mol %, 1 to 6 mol %, 1 to 7 mol %, 1 to 8 mol %, 1 to 9 mol %, 1 to 10 mol %, 1 to 15 mol %, 1 to 20 mol %, 1 to 25 mol %, 2 to 3 mol %, 2 to 4 mol %, 2 to 5 mol %, 2 to 6 mol %, 2 to 7 mol %, 2 to 8 mol %, 2 to 9 mol %, 2 to 10 mol %, 2 to 15 mol %, 2 to 25 mol %, 3 to 4 mol %, 3 to 5 mol %, 3 to 6 mol %, 3 to 7 mol %, 3 to 8 mol %, 3 to 9 mol %, 3 to 10 mol %, 3 to 15 mol %, 3 to 20 mol %, 3 to 25 mol %, 4 to 5 mol %, 4 to 6 mol %, 4 to 7 mol %, 4 to 8 mol %, 4 to 9 mol %, 4 to 10 mol %, 4 to 15 mol %, 4 to 20 mol %, 4 to 25 mol %, 5 to 10 mol %, 5 to 15 mol %, 5 to 20 mol %, 5 to 25 mol %, 10 to 15 mol %, 10 to 20 mol %, 10 to 25 mol %, 15 to 20 mol %, 15 to 25 mol %, and 20 to 25 mol %.

In some embodiments, the LNP is comprised of about 30-60 mol % of at least one cationic lipid, about 0-30 mol % of at least one non-cationic lipid (e.g., a phospholipid), about 18.5-48.5 mol % of at least one sterol (e.g., cholesterol), and about 0-10 mol % of at least one particle-activity-modifying-agent (e.g., a PEGylated lipid).

In some embodiments, the LNP is comprised of about 35-55 mol % of at least one cationic lipid, about 5-25 mol % of at least one non-cationic lipid (e.g., a phospholipid), about 30-40 mol % of at least one sterol (e.g., cholesterol), and about 0-10 mol % of at least one particle-activity-modifying-agent (e.g., a PEGylated lipid).

In some embodiments, the LNP is comprised of about 35-45 mol % of at least one cationic lipid, about 25-35 mol % of at least one non-cationic lipid (e.g., a phospholipid), about 20-30 mol % of at least one sterol (e.g., cholesterol), and about 0-10 mol % of at least one particle-activity-modifying-agent (e.g., a PEGylated lipid).

In some embodiments, the LNP is comprised of about 45-65 mol % of at least one cationic lipid, about 5-10 mol % of at least one non-cationic lipid (e.g., a phospholipid), about 25-40 mol % of at least one sterol (e.g., cholesterol), and about 0.5-10 mol % of at least one particle-activity-modifying-agent (e.g., a PEGylated lipid).

In some embodiments, the LNP is comprised of about 40-60 mol % of at least one cationic lipid, about 5-15 mol % of at least one non-cationic lipid (e.g., a phospholipid), about 35-45 mol % of at least one sterol (e.g., cholesterol), and about 0.5-3 mol % of at least one particle-activity-modifying-agent (e.g., a PEGylated lipid).

In some embodiments, the LNP is comprised of about 30-60 mol % of at least one cationic lipid, about 0-30 mol % of at least one non-cationic lipid (e.g., a phospholipid), about 15-50 mol % of at least one sterol (e.g., cholesterol), and about 0.01-10 mol % of at least one particle-activity-modifying-agent (e.g., a PEGylated lipid).

In some embodiments, the LNP is comprised of about 10-75 mol % of at least one cationic lipid, about 0.5-50 mol % of at least one non-cationic lipid (e.g., a phospholipid), about 5-60 mol % of at least one sterol (e.g., cholesterol), and about 0.1-20 mol % of at least one particle-activity-modifying-agent (e.g., a PEGylated lipid).

In some embodiments, the LNP is comprised of about 50-65 mol % of at least one cationic lipid, about 3-15 mol % of at least one non-cationic lipid (e.g., a phospholipid), about 30-40 mol % of at least one sterol (e.g., cholesterol), and about 0.5-2 mol % of at least one particle-activity-modifying-agent (e.g., a PEGylated lipid).

In some embodiments, the LNP is comprised of about 50-85 mol % of at least one cationic lipid, about 3-15 mol % of at least one non-cationic lipid (e.g., a phospholipid), about 30-40 mol % of at least one sterol (e.g., cholesterol), and about 0.5-2 mol % of at least one particle-activity-modifying-agent (e.g., a PEGylated lipid).

In some embodiments, the LNP is comprised of about 25-75 mol % of at least one cationic lipid, about 0.1-15 mol % of at least one non-cationic lipid (e.g., a phospholipid), about 5-50 mol % of at least one sterol (e.g., cholesterol), and about 0.5-20 mol % of at least one particle-activity-modifying-agent (e.g., a PEGylated lipid).

In some embodiments, the LNP is comprised of about 50-65 mol % of at least one cationic lipid, about 5-10 mol % of at least one non-cationic lipid (e.g., a phospholipid), about 25-35 mol % of at least one sterol (e.g., cholesterol), and about 5-10 mol % of at least one particle-activity-modifying-agent (e.g., a PEGylated lipid).

In some embodiments, the LNP is comprised of about 20-60 mol % of at least one cationic lipid, about 5-25 mol % of at least one non-cationic lipid (e.g., a phospholipid), about 25-55 mol % of at least one sterol (e.g., cholesterol), and about 0.5-15 mol % of at least one particle-activity-modifying-agent (e.g., a PEGylated lipid).

In some embodiments, the LNPs can be characterized by their shape. In some embodiments, the LNPs are essentially spherical. In some embodiments, the LNPs are essentially rod-shaped (e.g., cylindrical). In some embodiments, the LNPs are essentially disk shaped.

In some embodiments, the LNPs can be characterized by their size. In some embodiments, the size of an LNP can be defined as the diameter of its largest circular cross section, referred to herein simply as its diameter. In some embodiments the LNPs may have a diameter between 30 nm to about 150 nm. In some embodiments, the LNP may have diameters ranging between about 40 to 150 nm, 50 to 150 nm, 60 to 150 nm, about 70 to 150 nm, or 80 to 150 nm, 90 to 150 nm, 100 to nm, 110 to 150 nm, 120 to 150 nm, 130 to 150 nm, 140 to 150 nm, 30 to 150 nm, 40 to 140 nm, 40 to 140 nm, 50 to 140 nm, 60 to 140 nm, 70 to 140 nm, 80 to 140 nm, 90 to 140 nm, 100 to 140 nm, 110 to 140 nm, 120 to 140 nm, 130 to 140 nm, 30 to 140 nm, 40 to 130 nm, 50 to 130 nm, 60 to 130 nm, 70 to 130 nm, 80 to 130 nm, 90 to 130 nm, 100 to 130 nm, 110 to 130 nm, 120 to 130 nm, 30 to 120 nm, 40 to 120 nm, 50 to 120 nm, 60 to 120 nm, 70 to 120 nm, 80 to 120 nm, 90 to 120 nm, 100 to 120 nm, 110 to 120 nm, 30 to 110 nm, 40 to 110 nm, 50 to 110 nm, 60 to 110 nm, 70 to 110 nm, 80 to 110 nm, 90 to 110 nm, 100 to 110 nm, 30 to 100 nm, 40 to 100 nm, 50 to 100 nm, 60 to 100 nm, 70 to 100 nm, 80 to 100 nm, 90 to 100 nm, 30 to 90 nm, 40 to 90 nm, 50 to 90 nm, 60 to 90 nm, 70 to 90 nm, 80 to 90 nm, 30 to 80 nm, 40 to 80 nm, 50 to 80 nm, 60 to 80 nm, 70 to 80 nm, 30 to 70 nm, 40 to 70 nm, 50 to 70 nm, 60 to 70 nm, 30 to 60 nm, 40 to 60 nm, 50 to 60 nm, 30 to 50 nm, 40 to 50 nm, and 30 to 40 nm.

In some embodiments, a population of LNPs, such as those resulting from the same formulation, may be characterized by measuring the uniformity of size, shape, or mass of the particles in the population. uniformity may be expressed in some embodiments as the polydispersity index (PI) of the population. In some embodiments uniformity may be expressed in some embodiments as the disparity (D) of the population. The terms “polydispersity index” and “disparity” are understood herein to be equivalent and may be used interchangeably. In some embodiments, a population of LNPs resulting from a given formulation will have a PI of between about 0.1 and 1. In some embodiments, a population of LNPs resulting from a giving formulation will have a PI of less than about 1, less than about 0.5, less than about 0.4, less than about 0.3, less than about 0.2, less than about 0.1. In some embodiments, a population of LNPs resulting from a given formulation will have a PI of between about 0.1 to 1, 0.1 to 0.8, 0.1 to 0.6, 0.1 to 0.4, 0.1 to 0.2, 0.2 to 1, 0.2 to 0.8, 0.2 to 0.6, 0.2 to 0.4, 0.4 to 1, 0.4 to 0.8, 0.4 to 0.6, 0.6 to 1, 0.6 to 0.8, and 0.8 to 1.

In some embodiments, the LNP may fully or partially encapsulate a cargo, such as the originator constructs and benchmark constructs of the present disclosure. In some embodiments, essentially 0% of the cargo present in the final formulation is exposed to the environment outside of the LNP (e.g., the cargo is fully encapsulated. In some embodiments, the cargo is associated with the LNP but is at least partially exposed to the environment outside of the LNP. In some embodiments, the LNP may be characterized by the % of the cargo not exposed to the environment outside of the LNP, e.g., the encapsulation efficiency. For the sake of clarity, an encapsulation efficiency of about 100% refers to an LNP formulation where essentially all the cargo is fully encapsulated by the LNP, while an encapsulation rate of about 0% refers to an LNP where essential none of the cargo is encapsulated in the LNP, such as with an LNP where the cargo is bound to the external surface of the LNP. On some embodiments, an LNP may have an encapsulation efficiency of less than about 100%, less than about 95%, less than about 85%. less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15% less than about 10%, or less than 5%. In some embodiments, an LNP may have an encapsulation efficiency of between about 90 to 100%, 80 to 100%, 70 to 100%, 60 to 100%, 50 to 100%, 40 to 100%, 30 to 100%, 20 to 100%, 10 to 100%, 80 to 90%, 70 to 90%, 60 to 90%, 50 to 90%, 40 to 90%, 30 to 90%, 20 to 90%, 10 to 90%, 70 to 80%, 60 to 80%, 50 to 80%, 40 to 80%, 30 to 80%, 20 to 80%, 10 to 80%, 60 to 70%, 50 to 70%, 40 to 70%, 30 to 70%, 20 to 70%, 10 to 70%, 40 to 50%, 30 to 50%, 20 to 50%, 10 to 50%, 30 to 40%, 20 to 40%, 10 to 40%, 20 to 30%, 10 to 30%, and 10 to 20%.

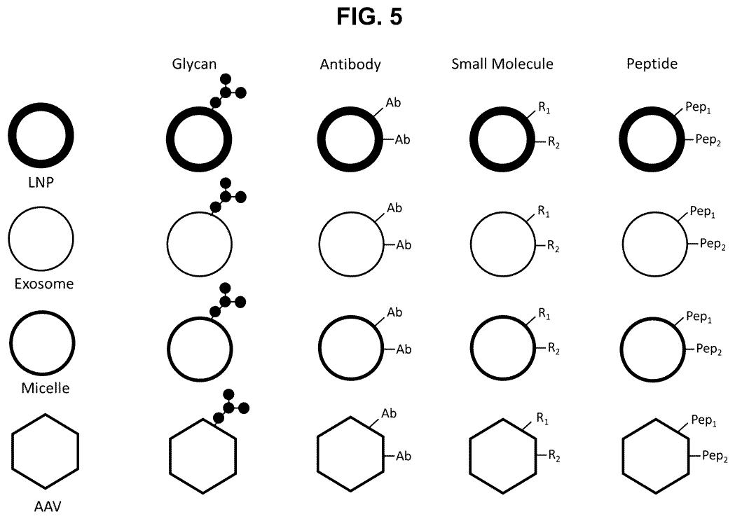

In some embodiments, a LNP may include at least one identifier moiety as shown in FIG. 5. Non-limiting examples of an identifier moiety include glycans, antibodies, peptides, small molecules, polynucleotides (e.g., circular DNA, circular RNA) and any combination thereof. In some embodiments, the at least one targeting agent may be incorporated into the lipid membrane of the lipid-based nanoparticle. In some embodiments, the at least one targeting agent may be presented on the external surface of the nanoparticle. In some embodiments, the at least one targeting agent may be conjugated to a lipid-component of the nanoparticle. In some embodiments, the at least one targeting agent may be conjugated to a polymer component of the nanoparticle. In some embodiments, the at least one targeting agent may be anchored to the nanoparticle via hydrophobic ad hydrophilic interactions among the at least one targeting agent, the nanoparticle membrane, and the aqueous environments inside or outside the nanoparticle. In some embodiments, the at least one targeting agent is conjugated to a peptide/protein component of the nanoparticle membrane. In some embodiments, the at least one targeting agent is conjugated to a suitable linker moiety which is conjugated to a component of the nanoparticle membrane. In some embodiments, any combination of forces and bonds can result in the targeting agent being associated with the nanoparticle.

The LNPs described herein may be formed using techniques known in the art. As a non-limiting example, an organic solution containing the lipids is mixed together with an acidic aqueous solution containing the originator construct or benchmark construct in a microfluidic channel resulting in the formation of targeting system (delivery vehicle and the benchmark construct).

In some embodiments, each LNP formulation includes a benchmark construct having a uniquely identifiable nucleotide identifier sequence (e.g., nucleotide barcode). The unique identifier sequence provides the ability to identify the specific LNP which produces the desired result. The LNP formulation may also differ in the LNP-forming composition used to generate the LNP. For example, the LNP-forming compositions can be varied in the molar amount and/or structure of the ionizable lipid, the molar amount and/or structure of the helper lipid, the molar amount/or structure of PEG, and/or the molar amount of cholesterol. Additionally, or alternatively, the LNP formulation may comprise benchmark constructs which differ in the coding sequence for the biologically active molecule. Additionally, or alternatively, the LNP formulation may comprise benchmark constructs which differ in the modifications made to the nucleic acid sequence.

In some embodiments, an LNP of the present disclosure comprises an ionizable lipid, a structural lipid, a PEGylated lipid (aka PEG lipid), and a phospholipid. In alternative embodiments, an LNP comprises an ionizable lipid, a structural lipid, a PEGylated lipid (aka PEG lipid), and a zwitterionic amino acid lipid. In some embodiments, an LNP further comprises a₅th lipid, besides any of the aforementioned lipid components. In some embodiments, the LNP encapsulates one or more elements of the active agent of the present disclosure. In some embodiments, an LNP further comprises a targeting moiety covalently or non-covalently bound to the outer surface of the LNP. In some embodiments, the targeting moiety is a targeting moiety that binds to, or otherwise facilitates uptake by, cells of a particular organ system.

In some embodiments, an LNP has a diameter of at least about 20 nm, 30 nm, 40 nm, 50 nm, 60 nm, 70 nm, 80 nm, or 90 nm. In some embodiments, an LNP has a diameter of less than about 100 nm, 110 nm, 120 nm, 130 nm, 140 nm, 150 nm, or 160 nm. In some embodiments, an LNP has a diameter of less than about 100 nm. In some embodiments, an LNP has a diameter of less than about 90 nm. In some embodiments, an LNP has a diameter of less than about 80 nm. In some embodiments, an LNP has a diameter of about 60-100 nm. In some embodiments, an LNP has a diameter of about 75-80 nm.

In some embodiments, the lipid nanoparticle compositions of the present disclosure are described according to the respective molar ratios of the component lipids in the formulation. As a non-limiting example, the mol-% of the ionizable lipid may be from about 10 mol-% to about 80 mol-%. As a non-limiting example, the mol-% of the ionizable lipid may be from about 20 mol-% to about 70 mol-%. As a non-limiting example, the mol-% of the ionizable lipid may be from about 30 mol-% to about 60 mol-%. As a non-limiting example, the mol-% of the ionizable lipid may be from about 35 mol-% to about 55 mol-%. As a non-limiting example, the mol-% of the ionizable lipid may be from about 40 mol-% to about 50 mol-%.

In some embodiments, the mol-% of the phospholipid may be from about 1 mol-% to about 50 mol-%. In some embodiments, the mol-% of the phospholipid may be from about 2 mol-% to about 45 mol-%. In some embodiments, the mol-% of the phospholipid may be from about 3 mol-% to about 40 mol-%. In some embodiments, the mol-% of the phospholipid may be from about 4 mol-% to about 35 mol-%. In some embodiments, the mol-% of the phospholipid may be from about 5 mol-% to about 30 mol-%. In some embodiments, the mol-% of the phospholipid may be from about 10 mol-% to about 20 mol-%. In some embodiments, the mol-% of the phospholipid may be from about 5 mol-% to about 20 mol-%.

In some embodiments, the mol-% of the structural lipid may be from about 10 mol-% to about 80 mol-%. In some embodiments, the mol-% of the structural lipid may be from about 20 mol-% to about 70 mol-%. In some embodiments, the mol-% of the structural lipid may be from about 30 mol-% to about 60 mol-%. In some embodiments, the mol-% of the structural lipid may be from about 35 mol-% to about 55 mol-%. In some embodiments, the mol-% of the structural lipid may be from about 40 mol-% to about 50 mol-%.

In some embodiments, the mol-% of the PEG lipid may be from about 0.1 mol-% to about 10 mol-%. In some embodiments, the mol-% of the PEG lipid may be from about 0.2 mol-% to about 5 mol-%. In some embodiments, the mol-% of the PEG lipid may be from about 0.5 mol-% to about 3 mol-%. In some embodiments, the mol-% of the PEG lipid may be from about 1 mol-% to about 2 mol-%. In some embodiments, the mol-% of the PEG lipid may be about 1.5 mol-%. a. Ionizable Lipids

In some embodiments, an LNP disclosed herein comprises an ionizable lipid. In some embodiments, an LNP comprises two or more ionizable lipids.

In some embodiments, an ionizable lipid has a dimethylamine or an ethanolamine head. In some embodiments, an ionizable lipid has an alkyl tail. In some embodiments, a tail has one or more ester linkages, which may enhance biodegradability. In some embodiments, a tail is branched, such as with 3 or more branches. In some embodiments, a branched tail may enhance endosomal escape. In some embodiments, an ionizable lipid has a pKa between 6 and 7, which may be measured, for example, by TNS assay.

In some embodiments, an ionizable lipid has a structure of any of the formulas disclosed below, and all formulas disclosed in a reference publication and patent application publication cited below. In some embodiments, an ionizable lipid comprises a head group of any structure or formula disclosed below. In some embodiments, an ionizable lipid comprises a bridging moiety of any structure or formula disclosed below. In some embodiments, an ionizable lipid comprises any tail group, or combination of tail groups disclosed below. The present disclosure contemplates all permutations and combinations of head group, bridging moiety and tail group, or tail groups, disclosed herein.

In some embodiments, a head, tail, or structure of an ionizable lipid is described in US patent application US20170210697A1.

In some embodiments, a compound has a structure according to formula 1:

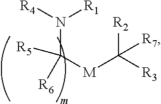

wherein:
R¹ is selected from the group consisting of C₅₋₃₀ alkyl, C₅₋₂₀ alkenyl,—R*YR″, YR″, and —R″M′R′;
R² and R³ are independently selected from the group consisting of H, C₁₋₁₄ alkyl, C₂₋₁₄ alkenyl, R*YR″, YR″, and R*OR″, or R² and R³, together with the atom to which they are attached, form a heterocycle or carbocycle;
R⁴ is selected from the group consisting of a C₃₋₆ carbocycle, (CH₂)nQ, (CH₂)nCHQR, —CHQR, CO(R)₂, and unsubstituted C₁₋₆ alkyl, where Q is selected from a carbocycle, heterocycle, —OR, O(CH₂)nN(R)₂, C(O)OR, OC(O)R, CX3, CX2H, —CXH2, —CN, N(R)₂, —C(O)N(R)₂, —N(R)C(O)R, —N(R)S(O)2R, —N(R)C(O)N(R)₂, —N(R)C(S)N(R)₂, —N(R)R⁸, —O(CH₂)ₙOR, —N(R)C(═NR⁹)N(R)₂—N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂—N(OR)C(—NR)N(R)—N(OR)C(═CHR⁹)N(R)₂, —C(═NR⁹)N(R)₂, C(═NR⁹)R, —C(O)N(R)OR, and C(R)N(R)₂, C(O)OR, and each n is independently selected from 1, 2, 3, 4, and 5 or a head group disclosed in Table 1; each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are independently selected from C(O)O—, —OC(O)—, —C(O)N(R′)—, —N(R′)C(O)—, —C(O)—, C(S)—, C(S)S—, SC(S)—, CH(OH)—, P(O)(OR′)O—, —S(O), S—S—, an aryl group, and a heteroaryl group;
R⁷ is selected from the group consisting of C1-3alkyl, C2-3 alkenyl, and H;
R⁸ is selected from the group consisting of C3-6 carbocycle and heterocycle;
R⁹ is selected from the group consisting of H. CN, NO₂, C₁₋₆ alkyl, —OR, S(O)₂R, S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, R*YR″, YR″, and H;
each R″ is independently selected from the group consisting of C₃₋₁₄ alkyl, C₃₋₁₄ alkenyl, and H;
each R* is independently selected from the group consisting of C₁₋₁₂ alkyl and C₂₋₁₂ alkenyl:
each Y is independently a C₃₋₆ carbocycle;
each X is independently selected from the group consisting of F, Cl, Br, and I;
each Q is —OH, —NHC(S)N(R)₂, —NHC(O)N(R)₂, —N(R)C(O)R, —N(R)S(O)₂R, —N(R)R′, —NHC(═NR⁹)N(R)₂, —NHC(═CHR⁹)N(R)₂, —OC(O)N(R)₂, —N(R)C(O)OR, heteroaryl or heterocycloalkyl; and
m is selected from 5, 6, 7, 8, 9, 10, 11, 12, and 13:
and wherein when R⁴ is (CH₂)nQ, (CH₂)nCHQR, CHQR, or CQ(R)₂, then (i) Q is not —N(R), when n is 1, 2, 3, 4 or 5, or (ii) Q is not 5, 6, or 7-membered heterocycloalkyl when n is 1 or 2.

In some embodiments, R⁴ is in Table 1.

In some embodiments, R⁴ in formula 1 is selected from head groups 1-47.

TABLE 1
Ionizable lipid head groups

| Head | |
|---|---|
| number | Structure |
| 1 | 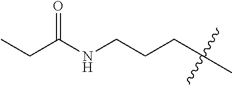 [SMILES: *CCCNC(=O)CC] |
| 2 | 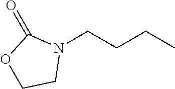 |
| 3 | 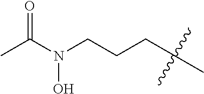 [SMILES: *CCCN(O)C(C)=O] |
| 4 | 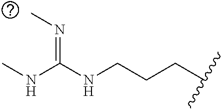 [SMILES: *CCCN/C(=N/C)NC] |
| 5 | 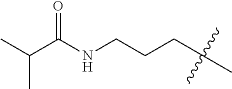 [SMILES: *CCCNC(=O)C(C)C] |
| 6 | 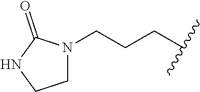 [SMILES: *CCCN1CCNC1=O] |
| 7 | 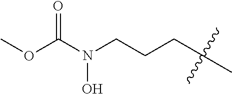 [SMILES: *CCCN(O)C(=O)OC] |
| 8 | 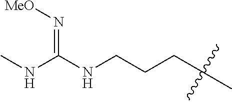 [SMILES: *CCCN/C(=N/OC)NC] |
| 9 | 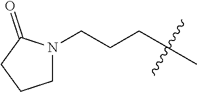 [SMILES: *CCCN1CCCC1=O] |
| 10 | 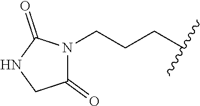 [SMILES: *CCCN1C(=O)CNC1=O] |
| 11 | 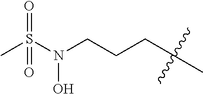 [SMILES: *CCCN(O)S(C)(=O)=O] |
| 12 | 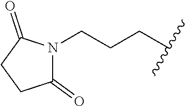 [SMILES: *CCCN1C(=O)CCC1=O] |
| 13 | 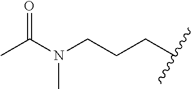 [SMILES: *CCCN(C)C(C)=O] |
| 14 | 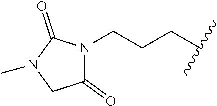 [SMILES: *CCCN1C(=O)CN(C)C1=O] |
| 15 | 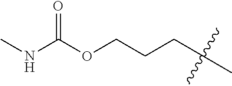 [SMILES: *CCCOC(=O)NC] |
| 16 | 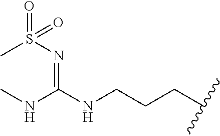 [SMILES: *CCCN/C(=N/S(C)(=O)=O)NC] |
| 17 | 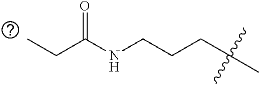 [SMILES: *CCCNC(=O)CC] |
| 18 | 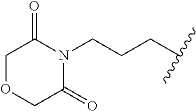 [SMILES: *CCCN1C(=O)COCC1=O] |
| 19 | 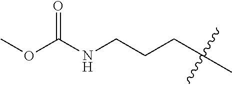 [SMILES: *CCCNC(=O)OC] |
| 20 | 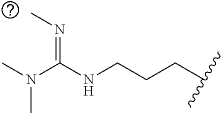 [SMILES: *CCCN/C(=N/C)N(C)C] |
| 21 | 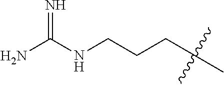 [SMILES: *CCCNC(=N)N] |
| 22 | 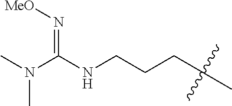 [SMILES: *CCCN/C(=N/OC)N(C)C] |
| 23 | 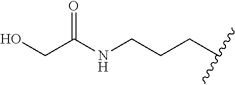 [SMILES: *CCCNC(=O)CO] |
| 24 | 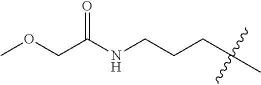 [SMILES: *CCCNC(=O)COC] |
| 25 | 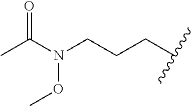 [SMILES: *CCCN(OC)C(C)=O] |
| 26 | 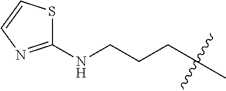 [SMILES: *CCCNc1nccs1] |
| 27 | 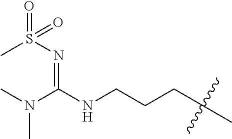 [SMILES: *CCCN/C(=N/S(C)(=O)=O)N(C)C] |
| 28 | 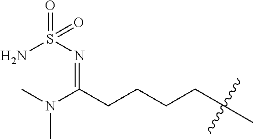 [SMILES: *CCCC/C(=N/S(N)(=O)=O)N(C)C] |
| 29 | 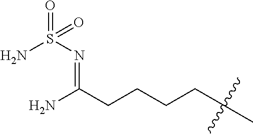 [SMILES: *CCCC/C(N)=N/S(N)(=O)=O] |
| 30 | 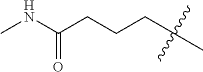 [SMILES: *CCCC(=O)NC] |
| 31 | 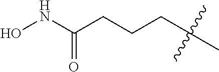 [SMILES: *CCCC(=O)NO] |
| 32 | 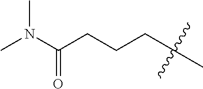 [SMILES: *CCCC(=O)N(C)C] |
| 33 | 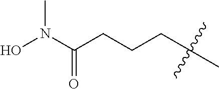 [SMILES: *CCCC(=O)N(C)O] |
| 34 | 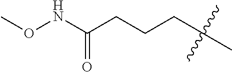 [SMILES: *CCCC(=O)NOC] |
| 35 | 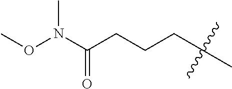 [SMILES: *CCCC(=O)N(C)OC] |
| 36 | 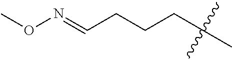 [SMILES: *CCC/C=N/OC] |
| 37 | 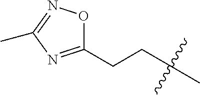 [SMILES: *CCc1nc(C)no1] |
| 38 | 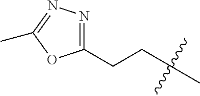 [SMILES: *CCc1nnc(C)o1] |
| 39 | 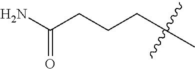 [SMILES: *CCCC(N)=O] |
| 40 | 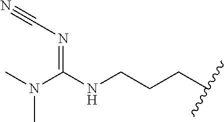 [SMILES: *CCCN/C(=N/C#N)N(C)C] |
| 41 | 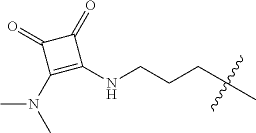 [SMILES: *CCCNc1c(N(C)C)c(=O)c1=O] |
| 42 | 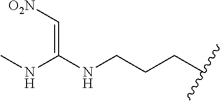 [SMILES: *CCCN/C(=C/[N+](=O)[O-])NC] |
| 43 | 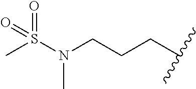 [SMILES: *CCCN(C)S(C)(=O)=O] |
| 44 | 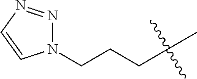 [SMILES: *CCCn1ccnn1] |
| 45 | 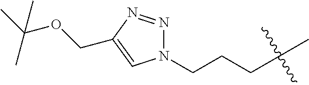 [SMILES: *CCCn1cc(COC(C)(C)C)nn1] |
| 46 | 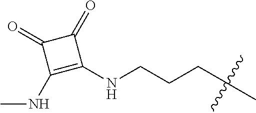 [SMILES: *CCCNc1c(NC)c(=O)c1=O] |
| 47 | 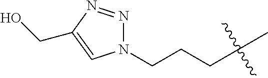 [SMILES: *CCCn1cc(CO)nn1] |
| 48 | 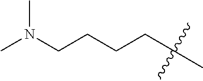 [SMILES: *CCCCN(C)C] |
| 49 | 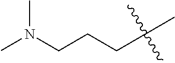 [SMILES: *CCCN(C)C] |
| 50 | 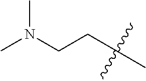 [SMILES: *CCN(C)C] |
| 51 | 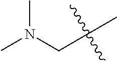 [SMILES: *CN(C)C] |
| 52 | 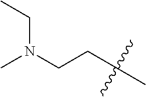 [SMILES: *CCN(C)CC] |
| 53 | 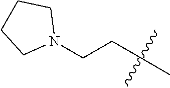 [SMILES: *CCN1CCCC1] |
| 54 | 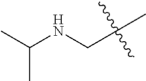 [SMILES: *CNC(C)C] |
| 55 | 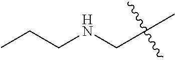 [SMILES: *CNCCC] |
| 56 | 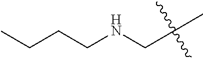 [SMILES: *CNCCCC] |
| 57 | 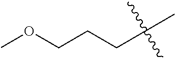 [SMILES: *CCCOC] |
| 58 | 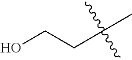 [SMILES: *CCO] |
| 59 | 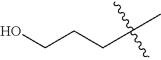 [SMILES: *CCCO] |
| 60 | 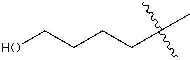 [SMILES: *CCCCO] |
| 61 | 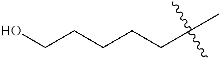 [SMILES: *CCCCCO] |
| 62 | 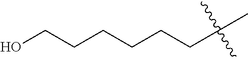 [SMILES: *CCCCCCO] |
| 63 | 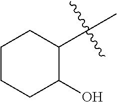 [SMILES: *C1CCCCC1O] |
| 64 | 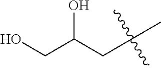 [SMILES: *CC(O)CO] |
| 65 | 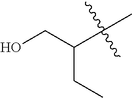 [SMILES: *C(CC)CO] |
| 66 | 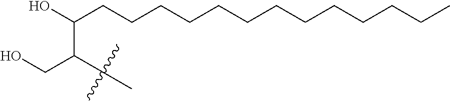 [SMILES: *C(CO)C(O)CCCCCCCCCCCCC] |
| 67 | 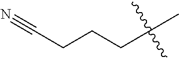 [SMILES: *CCCC#N] |
| 68 | 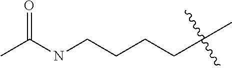 [SMILES: *CCCCNC(C)=O] |

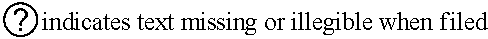

In some embodiments, a subset of the compounds of formula 1 are also described by formula 1b:

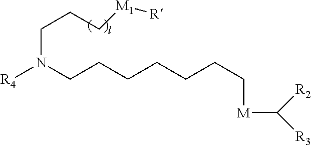

wherein 1 is selected from 1, 2, 3, 4, and 5; M¹ is a bond or M′; R⁴ is unsubstituted C₁₋₃ alkyl, or —(CH₂)nQ, in which n is 2, 3, or 4, and Q is —OH, —NHC(S)N(R)₂, —NHC(O)N(R)₂, —N(R)C(O)R, —N(R)S(O)₂R, —N(R)R′, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R² and R3 are independently selected from the group consisting of H, C₁₋₁₄ alkyl, and C₂₋₁₄ alkenyl.

In some embodiments, a head, tail, or structure of an ionizable lipid is described in international patent application PCT/US2018/058555.

In some embodiments, an ionizable lipid has a structure according to formula 2:

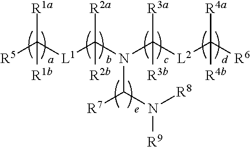

wherein:
one of L1 or L² is —O(C═O)—, —(C═O)O—, —C(═O)—, —O—, —S(O)_{X}—, —S—S—, —C(═O)S—, SC(═O)—, —NRdC(═O)—, —C(═O)NRa—, NRdC(═O)NRa—, —OC(═O)NRa— or —NRdC(═O)O—, and the other of L1 or L² is —O(C═O)—, —(C═O)O—, —C(═O)—, —O—, —S(O)_{X}—, —S—S—, —C(═O)S—, SC(═O)—, —NRdC(═O)—, —C(═O)NRa—, NRdC(═O)NRa—, —OC(═O)NRa— or —NRdC(═O)O— or a direct bond;
Ra is H or C1-C12 alkyl;
R^{1a} and R^{1b} are, at each occurrence, independently either (a) H or C1-C12 alkyl, or (b) R^{1a} is H or C1-C12 alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C1-C12 alkyl, or (b) R^{2a} is H or C1-C12 alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C1-C12 alkyl, or (b) R^{3a} is H or C1-C12 alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C1-C12 alkyl, or (b) R^{4a} is H or C1-C12 alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C1-C12 alkyl;
R⁸ and R⁹ are each independently unsubstituted C1-C12 alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24;
e is 1 or 2; and
x is 0, 1 or 2.

In some embodiments, an ionizable lipid has a structure according to formula 3:

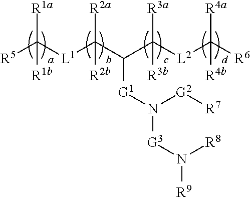

wherein:
one of L¹ or L² is —O(C═O)—, —(C═O)O—, —C(═O)—, —O—, —S(O)_{X}—, —S—S—, —C(═O)S—, SC(═O)—, —NRdC(═O)—, —C(═O)NRa—, NRdC(═O)NRa—, —OC(═O)NRa— or —NRdC(═O)O—, and the other of L¹ or L² is —O(C═O)—, —(C═O)O—, —C(═O)—, —O—, —S(O)_{X}—, —S—S—, —C(═O)S—, SC(═O)—, —NRdC(═O)—, —C(═O)NRa—, NRdC(═O)NRa—, —OC(═O)NRa— or —NRdC(═O)O— or a direct bond;
G1 is C1-C2 alkylene, —(C═O)—, —O(C═O)—, —SC(═O)—, —NRdC(═O)— or a direct bond:
G2 is —C(═O)—, —(C═O)O—, —C(═O)S—, —C(═O)NRa— or a direct bond;
G3 is C1-C6 alkylene;
Ra is H or C1-C12 alkyl;
R^{1a} and R^{1b} are, at each occurrence, independently either: (a) H or C1-C12 alkyl; or (b) R^{1a} is H or C1-C12 alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C1-C12 alkyl; or (b) R^{2a} is H or C1-C12 alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C1-C12 alkyl; or (b) R^{3a} is H or C1-C12 alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C1-C12 alkyl; or (b) R^{4a} is H or C1-C12 alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C4-C20 alkyl;
R⁸ and R⁹ are each independently C1-C12 alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

In some embodiments, an ionizable lipid has a structure according to formula 4:

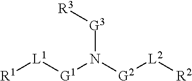

wherein:
one of L¹ or L² is —O(C═O)—, —(C═O)O—, —C(═O)—, —O—, —S(O)_{X}—, —S—S—, —C(═O)S—, SC(═O)—, —NRdC(═O)—, —C(═O)NRa—, NRdC(═O)NRa—, —OC(═O)NRa— or —NRdC(═O)O—, and the other of L¹ or L² is —O(C═O)—, —(C═O)O—, —C(═O)—, —O—, —S(O)_{X}—, —S—S—, —C(═O)S—, SC(═O)—, —NRdC(═O)—, —C(═O)NRa—, NRdC(═O)NRa—, —OC(═O)NRa— or —NRdC(═O)O— or a direct bond;
G¹ and G² are each independently unsubstituted C1-C12 alkylene or C1-C12 alkenylene;
G³ is C1-C24 alkylene, C1-C24 alkenylene, C3-C8 cycloalkylene, C3-C8 cycloalkenylene;
Ra is H or C1-C12 alkyl;
R¹ and R² are each independently C6-C24 alkyl or C6-C24 alkenyl;
R³ is H, OR5, CN, —C(═O)OR4, —OC(═O)R4 or —NR5C(═O)R4;
R⁴ is C1-C12 alkyl;
R⁵ is H or C1-C6 alkyl; and
x is 0, 1 or 2.

In some embodiments, an ionizable lipid has a structure according to formula 5:

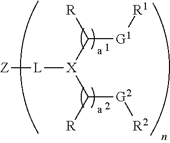

wherein:
one of G¹ or G² is, at each occurrence, —O(C═O)—, —(C═O)O—, —C(═O)—, —O—, —S(O)y, —S—S—, —C(═O)S—, SC(═O)—, —N(Ra)C(═O)—, —C(═O)N(Ra)—, —N(Ra)C(═O)N(Ra)—, —OC(═O)N(Ra)— or —N(Ra)C(═O)O—, and the other of G1 or G2 is, at each occurrence, —O(C═O)—, —(C═O)O—, —C(═O)—, —O—, —S(O)y, —S—S—, —C(═O)S—, —SC(═O)—, —N(Ra)C(═O)—, —C(═O)N(Ra)—, —N(Ra)C(═O)N(Ra)—, —OC(═O)N(Ra)— or —N(Ra)C(═O)O— or a direct bond;
L is, at each occurrence, ˜O(C═O)—, wherein ˜ represents a covalent bond to X;
X is CRa;
Z is alkyl, cycloalkyl or a monovalent moiety comprising at least one polar functional group when n is 1; or Z is alkylene, cycloalkylene or a polyvalent moiety comprising at least one polar functional group when n is greater than 1;
Ra is, at each occurrence, independently H, C1-C12 alkyl, C1-C12 hydroxylalkyl, C1-C12 aminoalkyl, C1-C12 alkylaminylalkyl, C1-C12 alkoxyalkyl, C1-C12 alkoxycarbonyl, C1-C12 alkylcarbonyloxy, C1-C12 alkylcarbonyloxyalkyl or C1-C12 alkylcarbonyl;
R is, at each occurrence, independently either: (a) H or C1-C12 alkyl; or (b) R together with the carbon atom to which it is bound is taken together with an adjacent R and the carbon atom to which it is bound to form a carbon-carbon double bond;
R¹ and R² have, at each occurrence, the following structure, respectively:

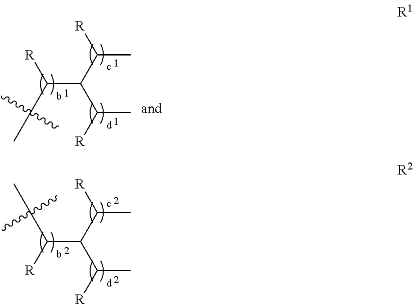

a¹ and a² are, at each occurrence, independently an integer from 3 to 12;
b¹ and b² are, at each occurrence, independently 0 or 1;
c¹ and c² are, at each occurrence, independently an integer from 5 to 10;
d¹ and d² are, at each occurrence, independently an integer from 5 to 10;
y is, at each occurrence, independently an integer from 0 to 2; and
n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, an ionizable lipid has a structure according to formula 6:

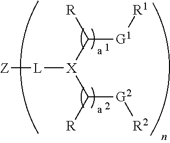

wherein:
one of G¹ or G² is, at each occurrence, —O(C═O)—, —(C═O)O—, —C(═O)—, —O—, —S(O)y, —S—S—, —C(═O)S—, SC(═O)—, —N(Ra)C(═O)—, —C(═O)N(Ra)—, —N(Ra)C(═O)N(Ra)—, —OC(═O)N(Ra)— or —N(Ra)C(═O)O—, and the other of G1 or G2 is, at each occurrence, —O(C═O)—, —(C═O)O—, —C(═O)—, —O—, —S(O)y-, —S—S—, —C(═O)S—, —SC(═O)—, —N(Ra)C(═O)—, —C(═O)N(Ra)—, —N(Ra)C(═O)N(Ra)—, —OC(═O)N(Ra)— or —N(Ra)C(═O)O— or a direct bond;
L is, at each occurrence, ˜O(C═O)—, wherein ˜ represents a covalent bond to X;
X is CRa;
Z is alkyl, cycloalkyl or a monovalent moiety comprising at least one polar functional group when n is 1; or Z is alkylene, cycloalkylene or a polyvalent moiety comprising at least one polar functional group when n is greater than 1;
Ra is, at each occurrence, independently H, C1-C12 alkyl, C1-C12 hydroxylalkyl, C1-C12 aminoalkyl, C1-C12 alkylaminylalkyl, C1-C12 alkoxyalkyl, C1-C12 alkoxycarbonyl, C1-C12 alkylcarbonyloxy, C1-C12 alkylcarbonyloxyalkyl or C1-C12 alkylcarbonyl;
R is, at each occurrence, independently either: (a) H or C1-C12 alkyl; or (b) R together with the carbon atom to which it is bound is taken together with an adjacent R and the carbon atom to which it is bound to form a carbon-carbon double bond;
R¹ and R² have, at each occurrence, the following structure, respectively:

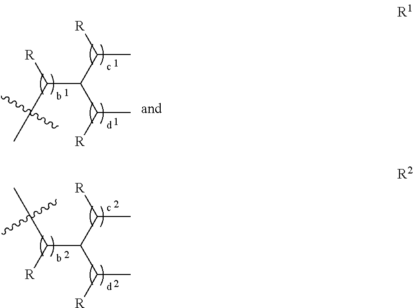

R′ is, at each occurrence, independently H or C1-C12 alkyl;
a¹ and a² are, at each occurrence, independently an integer from 3 to 12;
b¹ and b² are, at each occurrence, independently 0 or 1;
c¹ and c² are, at each occurrence, independently an integer from 2 to 12;
d¹ and d² are, at each occurrence, independently an integer from 2 to 12;
y is, at each occurrence, independently an integer from 0 to 2; and
n is an integer from 1 to 6,
wherein a¹, a², c¹, c², d¹ and d² are selected such that the sum of a¹+c¹+d¹ is an integer from 18 to 30, and the sum of a²+c²+d² is an integer from 18 to 30, and wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.
   In certain embodiments of Formula (V), G¹ and G² are each independently —O(C═O)— or —(C═O)O—.
   In some embodiments, an ionizable lipid has a disulfide tail.
   In some embodiments, an ionizable lipid includes short peptides of 12-15 mer length as head groups.
   In some embodiments, the head of an ionizable lipid comprises the structure of Vitamin A, D, E, or K as described in the published Patent Application WO2019232095A1, which is incorporated by herein by reference in its entirety.

In some embodiments, a lipid is described in international patent applications WO2021077067, or WO2019152557, each of which is incorporated herein by reference in its entirety.

In some embodiments, an LNP described herein comprises a lipid, e.g., an ionizable lipid, disclosed in US 2019/0240354, which is incorporated herein by reference in its entirety.

In some embodiments, the lipids disclosed in US 2019/0240354 are of Formula I:

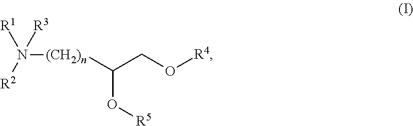

or salts thereof, wherein:
R¹ and R² are either the same or different and are independently hydrogen (H) or an optionally substituted C₁-C₆ alkyl, C₂-C₆ alkenyl, or C₂-C₆ alkynyl, or R¹ and R²may join to form an optionally substituted heterocyclic ring of 4 to 6 carbon atoms and 1 or 2 heteroatoms selected from the group consisting of nitrogen (N), oxygen (O), and mixtures thereof;
R³ is either absent or is hydrogen (H) or a C1-C6 alkyl to provide a quaternary amine; R⁴ and R⁵ are either the same or different and are independently an optionally substituted C₁₀-C₂₄ alkyl, C₁₀-C₂₄ alkenyl, C₁₀-C₂₄ alkynyl, or C₁₀-C₂₄ acyl, wherein at least one of R⁴ and R⁵ comprises at least two sites of unsaturation; and
n is 0, 1, 2, 3, or 4.

In some embodiments, the lipids disclosed in US 2019/0240354 are of Formula II:

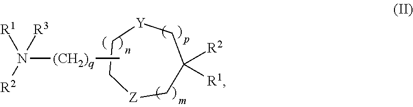

wherein R¹ and R are either the same or different and are independently an optionally substituted C₁₂-C₂₄ alkyl, C₁₂-C₂₄ alkenyl, C₁₂-C₂₄ alkynyl, or C₁₂-C₂₄ acyl; R³ and R⁴ are either the same or different and are independently an optionally substituted C₁-C₆ alkyl, C₂-C₆ alkenyl, or C₂-C₆ alkynyl, or R³ and R⁴ may join to form an optionally substituted heterocyclic ring of 4 to 6 carbon atoms and 1 or 2 heteroatoms chosen from nitrogen and oxygen; R⁵ is either absent or is hydrogen (H) or a C₁-C₆ alkyl to provide a quaternary amine; m, n, and p are either the same or different and are independently either 0, 1, or 2, with the proviso that m, n, and p are not simultaneously 0; q is 0, 1, 2, 3, or 4; and Y and Z are either the same or different and are independently O, S, or NH. In some embodiments, q is 2.

In some embodiments, the cationic lipid of Formula II is 2,2-dilinoleyl-4-(2-dimethylaminoethyl)-[1,3]-dioxolane, 2,2-dilinoleyl-4-(3-dimethylaminopropyl)-[1,3]-dioxolane, 2,2-dilinoleyl-4-(4-dimethylaminobutyl)-[1,3]-dioxolane, 2,2-dilinoleyl-5-dimethylaminomethyl-[1,3]-dioxane, 2,2-dilinoleyl-4-N-methylpepiazino-[1,3]-dioxolane, 2,2-dilinoleyl-4-dimethylaminomethyl-[1,3]-dioxolane, 2,2-dioleoyl-4-dimethylaminomethyl-[1,3]-dioxolane, 2,2-distearoyl-4-dimethylaminomethyl-[1,3]-dioxolane, 2,2-dilinoleyl-4-N-morpholino-[1,3]-dioxolane, 2,2-Dilinoleyl-4-trimethylamino-[1,3]-dioxolane chloride, 2,2-dilinoleyl-4,5-bis(dimethylaminomethyl)-[1,3]-dioxolane, 2,2-dilinoleyl-4-methylpiperzine-[1,3]-dioxolane, or mixtures thereof. In some embodiments, the cationic lipid of Formula II is 2,2-dilinoleyl-4-(2-dimethylaminoethyl)-[1,3]-dioxolane.

In some embodiments, the lipids disclosed in US 2019/0240354 are of Formula III:

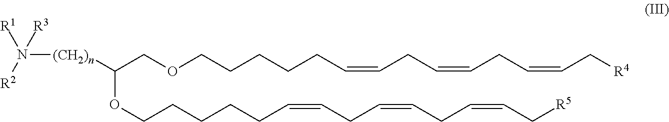

or salts thereof, wherein: R¹ and R² are either the same or different and are independently an optionally substituted C₁-C₆ alkyl, C₂-C₆ alkenyl, or C₂-C₆ alkynyl, or R¹ and R²may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either absent or present and when present are either the same or different and are independently an optionally substituted C₁-C₁₀ alkyl or C₂-C₁₀ alkenyl; and n is 0, 1, 2, 3, or 4.

In some embodiments, the lipids disclosed in US 2019/0240354 are of Formula C:

X-A-Y—Z¹; (Formula C)

or salts thereof, wherein:
X is N(H)R or NR₂;
A is absent, C₁ to C₆ alkyl, C₂to C₆ alkenyl, or C₂to C₆ alkynyl, which C₁ to C₆ alkyl, C₂to C₆ alkenyl, and C₂ to C₆ alkynyl is optionally substituted with one or more groups independently selected from oxo, halogen, heterocycle, CN, OR^{x}, NR^{x}Ry, NR^{x}C(═O)R^{y}, NRxSO₂R^{y}, C(═O)R^{x}, C(═O)OR^{x}, C(═O)NR^{x}R^{y}, SOnR^{x}, and SOnNR^{x}R^{y}, wherein n is 0, 1, or 2, and R^{x} and R are each independently hydrogen, alkyl, or heterocycle, wherein each alkyl and heterocycle of R^{x} and R may be further substituted with one or more groups independently selected from oxo, halogen, OH, CN, alkyl, OR^{x′}, heterocycle, NR^{x′}R^{y′}, —NR^{x′}C(═O)R^{y′}, NR^{x′}SO₂R^{y′}, C(═O)R^{x′}, C(═O)OR^{x′}, C(═O)NR^{x′}R^{y′}, SO_{n′}R^{x′}, and SO_{n′}NR^{x′}R^{y′}, wherein n′ is 0, 1, or 2, and R^{x′} and R^{y′}are each independently hydrogen, alkyl, or heterocycle;
Y is selected from the group consisting of absent, C(═O)—, —O—, OC(═O) C(═O)O—, N(Rb)C(═O)—, C(═O)N(Rb)—, N(Rb)C(═O)O—, and OC(═O)N(Rb)—;
Z¹ is a C₁ to C₆ alkyl that is substituted with three or four R^{x} groups, wherein each RX is independently selected from C₆ to C₁₁ alkyl, C₆ to C₁₁ alkenyl, and C₆ to C₁₁ alkynyl, which C₆ to C₁₁ alkyl, C₆to C₁₁ alkenyl, and C₆to C₁₁ alkynyl is optionally substituted with one or more groups independently selected from oxo, halogen, heterocycle, CN, OR^{x}, NR^{x}R^{y}, NR^{x}C(═O)R^{y}, NR^{x}SO₂R^{Y}, C(═O)R^{x}, C(═O)OR^{x}, C(═O)NR^{x}R^{y}, SOₙR^{x}, and SOₙNR^{x}R^{y}, wherein n is 0, 1, or 2, and R^{x} and R are each independently hydrogen, alkyl, or heterocycle, wherein any alkyl and heterocycle of R^{x} and R may be further substituted with one or more groups independently selected from oxo, halogen, OH, CN, alkyl, OR^{x′}, heterocycle, NR^{x}R^{y′}, —NR^{x′}C(═O)R^{y′}, NR^{x}SO₂R^{y′}, C(═O)R^{x′}, C(═O)OR^{x′}, C(═O)NR^{x′}R^{y′}, SO_{n′}R^{x′}, and SO_{n′}NR^{x′}R^{y′}, wherein n′ is 0, 1, or 2, and R^{x′} and R^{y′}are each independently hydrogen, alkyl, or heterocycle;
each R is independently alkyl, alkenyl, or alkynyl, that is optionally substituted with one or more groups independently selected from oxo, halogen, heterocycle, CN, OR′, —NR^{x}R NR^{x}C(═O)R^{y}, —NR^{x}SO₂R^{y}, C(═O)R^{x}, C(═O)OR^{x}, C(═O)NR^{x}R^{y}, SOₙR^{x}, and SOₙNR^{x}R^{y}, wherein n is 0, 1, or 2, and R^{x} and R^{y} are each independently hydrogen, alkyl, or heterocycle, wherein any alkyl and heterocycle of R and R may be further substituted with one or more groups independently selected from oxo, halogen, OH, CN, alkyl, OR^{x′}, heterocycle, NR^{x′}R^{y′}, —NR^{x′}C(═O)R^{y′}, NR^{x}SO₂R^{y′}, C(═O)R′, C(═O)OR^{x′}, C(═O)NR^{x′}R^{y′}, SO_{n′}R^{x′}, and SO_{n′}NR^{x′}R^{y′}, wherein n′ is 0, 1, or 2, and R^{x′} and R^{y′}are each independently hydrogen, alkyl, or heterocycle; and
each R^{b} is H or C₁ to C₆alkyl.

In some embodiments, an LNP described herein comprises a lipid, e.g., an ionizable lipid, disclosed in US 2010/0130588, which is incorporated herein by reference in its entirety. In some embodiments, the lipids disclosed in US 2010/0130588 are of Formula I:

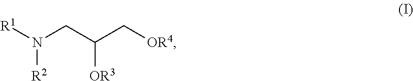

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation. In some embodiments, R³ and R⁴ are both the same, i.e., R³ and R⁴ are both linoleyl (Cis), etc. In some embodiments, R³ and R⁴ are different, i.e., R³ is tetradectrienyl (C₁₄) and R⁴ is linoleyl (Cis).

In some embodiments, the lipid of Formula I is 1,2-dilinoleyloxy-N,N-dimethylaminopropane (DLinDMA) or 1,2-dilinolenyloxy-N,N-dimethylaminopropane (DLenDMA).

In some embodiments, the lipids disclosed in US 2010/0130588 are of Formula II:

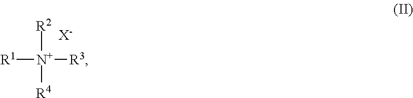

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation.

In some embodiments, an LNP described herein comprises a lipid, e.g., an ionizable lipid, disclosed in US 2021/0087135, which is incorporated herein by reference in its entirety.

In some embodiments, the lipids disclosed in US 2021/0087135 are of Formula (A):

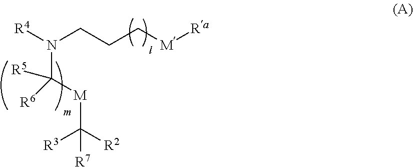

or its N-oxide, or a salt or isomer thereof,
wherein R^{′a} is R^{′branched} or R^{′cyclic}; wherein
R^{′branched} is:

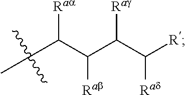

R^{′cyclic} is:

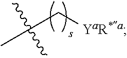

wherein:

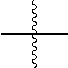

denotes a point of attachment;
wherein R^{a α}is H, and R^{aβ}, R^{aγ}, and R^{aδ}are each independently selected from the group consisting of H, C₂₋₁₂ alkyl, and C₂₋₁₂ alkenyl, wherein at least one of R^{aβ}, R^{aγ}, and R^{aδ}is selected from the group consisting of C₂₋₁₂ alkyl and C₂₋₁₂ alkenyl;
R² and R³ are each C₁₋₁₄ alkyl;
R⁴ is selected from the group consisting of (CH₂)₂OH, (CH₂)₃₀H, (CH₂)₄OH, (CH₂)₅OH and
wherein:

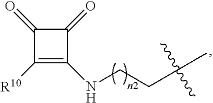

denotes a point of attachment;

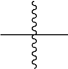

R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
R⁷ is H;
M and M′ are each independently selected from the group consisting of C(O)O and OC(O);
R′ is a C₁₋₁₂ alkyl or C₂₋₁₂ alkenyl;
Y^{a} is a C₃₋₆ carbocycle;
R*^{″a} is selected from the group consisting of C₁₋₁₅ alkyl and C₂₋₁₅ alkenyl;
1 is selected from the group consisting of 1, 2, 3, 4, and 5;
s is 2 or 3; and
m is selected from the group consisting of 5, 6, 7, 8, 9, 10, 11, 12, and 13.

In some embodiments, an LNP described herein comprises a lipid, e.g., an ionizable lipid, disclosed in US 2021/0128488, which is incorporated herein by reference in its entirety In some embodiments, the lipids disclosed in US 2021/0128488 are of structure (I):

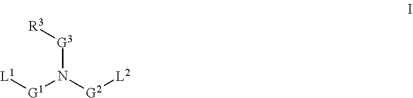

or a pharmaceutically acceptable salt, prodrug or stereoisomer thereof, wherein:
L¹ is O(C═O)R′, (C═O)OR¹, C(═O)R¹, OR¹, —S(O)ₓR¹, —S—SR¹, C(′O)SR′, SC(═O)R′, NR^{a}C(═O)R¹, C(═O)NR^{b}R^{c}, —NR^{a}C(═O)NR^{b}R^{c}, OC(═O)NR^{b}R^{c} or NR^{d}C(═O)OR¹;
L² is O(C═O)R², (C═O)OR², C(═O)R², OR², —S(O)ₓR², S—SR², C(═O)SR², SC(═O)R², NR^{d}C(═O)R², C(═O)NR^{e}RW, NR^{f}C(═O)NR^{c}R, OC(═O)NR^{c}R; NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{c} are each independently H or C₁-C₁₂ alkyl or C₁-C₁₂ alkenyl;
R^{c} and R are each independently C₁-C₁₂ alkyl or C₂-C₁₂ alkenyl;
R¹ and R² are each independently branched C₆-C₂₄ alkyl or branched C₆-C₂₄ alkenyl;
R³ is N(R⁴)R⁵;
R⁴ is C₁-C₁₂ alkyl;
R⁵ is substituted C₁-C₁₂ alkyl; and
x is 0, 1 or 2, and wherein each alkyl, alkenyl, alkylene, alkenylene, cycloalkylene, cycloalkenylene, aryl and aralkyl is independently substituted or unsubstituted unless otherwise specified.

In some embodiments, an LNP described herein comprises a lipid, e.g., an ionizable lipid, disclosed in US 2020/0121809, which is incorporated herein by reference in its entirety.

In some embodiments the lipids disclosed in US 2020/0121809 have a structure of Formula II:

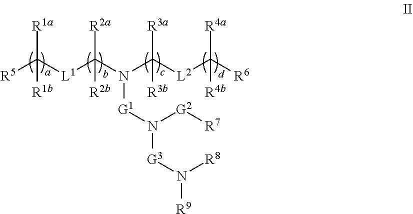

or a pharmaceutically acceptable salt, tautomer, prodrug or stereoisomer thereof, wherein:
one of L¹ or L² is O(C═O)—, (C═O)O—, C(═O), —O—, —S(O)_{X}—, —S—S—, C(═O)S—, SC(═O)—, —NR^{a}C(═O)—, C(═O)NR^{a}—, NR^{a}C(═)NR^{a}, OC(═O)NR^{a}— or —NR^{a}C(═O)O—, and the other of L¹ or L² is O(C═O)—, (C═O)O—, C(═O)—, —O—, S(O)ₓ—, —S—S, C(═O)S, SC(═O)—, NR^{a}C(═O)—, C(═O)NR^{a}—, NR^{a}C(═O)NR^{a}OC(═O)NR^{a}— or NR^{a}C(═O)O or a direct bond;
G¹ is C₁-C₂ alkylene, (C═O)—, O(C═O)—, SC(═O)—, NR^{a}C(═O) or a direct bond;
G² is C(═O)—, (C═O)O—, C(═O)S—, C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In some embodiments, the lipids disclosed in US 2020/0121809 have a structure of Formula III:

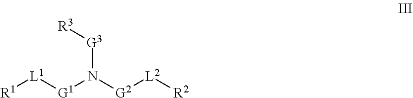

or a pharmaceutically acceptable salt, prodrug or stereoisomer thereof, wherein:
one of L¹ or L² is O(C═O)—, (C═O)O—, C(═O), —O—, —S(O)ₓ—, —S—S—, C(═O)S, SC(═O)—, NR^{a}C(═O)—, C(═O)NR^{a}—, NR^{a}C(═O)NR^{a}—, OC(═O)NR^{a}— or NR^{a}C(═O)O—, and the other of L¹ or L² is O(C═O)—, (C═O)O—, C(═O)—, —O—, S(O)ₓ, —S—S, C(═O)S, SC(═O)—, NR^{a}C(═O)—, C(═O)NR^{a}—, NR^{a}C(═O)NR^{a}OC(═O)NR^{a}— or NR^{a}C(═O)O or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR^{S}, CN, C(═O)OR⁴, OC(═O)R⁴ or NR⁵C(═O)R⁴;
R⁴ is C₁-C₁₂ alkyl;
R⁵ is H or C₁-C₆ alkyl; and
x is 0, 1 or 2.

In some embodiments, the lipids disclosed in US 2020/0121809 have a structure of Formula (IV):

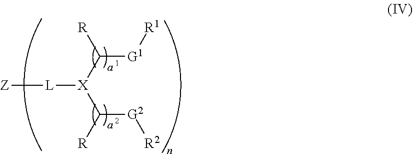

or a pharmaceutically acceptable salt, prodrug or stereoisomer thereof, wherein:
one of G¹ or G² is, at each occurrence, O(C═O)—, (C═O)O, C(═O), —O—, S(O)y, —SS, C(═O)S—, SC(═O)—, N(R^{a})C(═O)—, C(═O)N(R^{a}) N(R^{a})C(═O)N(R^{a})—, OC(═O)N(R^{a}) or N(R^{a})C(═O)O—, and the other of G¹ or G² is, at each occurrence, O(C═O)—, (C═O)O—, C(═O)—, —O—, —S(O)_{y}—, S—S—, C(═O)S, SC(═O)—, N(R^{a})C(═O)—, C(═O)N(R^{a})—, N(R^{a})C(═O)N(R^{a})—, OC(═O)N(R^{a}) or N(R^{a})C(═O)O or a direct bond;
L is, at each occurrence, O(C═O)—, wherein — represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂alkyl, C₁-C₁₂hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R²have, at each occurrence, the following structure, respectively:

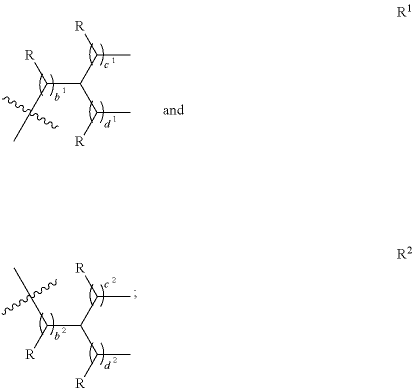

a¹ and a² are, at each occurrence, independently an integer from 3 to 12;
b¹ and b² are, at each occurrence, independently 0 or 1;
c¹ and c² are, at each occurrence, independently an integer from 5 to 10;
d¹ and d² are, at each occurrence, independently an integer from 5 to 10;
y is, at each occurrence, independently an integer from 0 to 2; and
n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, an LNP described herein comprises a lipid, e.g., an ionizable lipid, disclosed in US 2013/0108685, which is incorporated herein by reference in its entirety.

In some embodiments, the lipids disclosed in US 2013/0108685 are represented by the following formula (I):

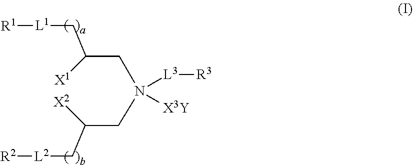

wherein:
R¹ and R² are, the same or different, each linear or branched alkyl, alkenyl or alkynyl having 12 to 24 carbon atoms, or R¹ and R²are combined together to form dialkylmethylene, dialkenylmethylene, dialkynylmethylene or alkylalkenylmethylene, X¹ and X³ are hydrogen atoms, or are combined together to form a single bond or alkylene, X³ is absent or represents alkyl having 1 to 6 carbon atoms, or alkenyl having 3 to 6 carbon atoms,
when X³ is absent,
Y is absent, a and b are 0, L³ is a single bond, R³ is alkyl having 1 to 6 carbon atoms, alkenyl having 3 to 6 carbon atoms, pyrrolidin-3-yl, piperidin-3-yl, piperidin-4-yl, or alkyl having 1 to 6 carbon atoms or alkenyl having 3 to 6 carbon atoms substituted with 1 to 3 substituent(s), which is(are), the same or different, amino, monoalkylamino, dialkylamino, trialkylammonio, hydroxy, alkoxy, carbamoyl, monoalkylcarbamoyl, dialkylcarbamoyl, pyrrolidinyl, piperidyl or morpholinyl, and L¹ and L²are —O—, Y is absent, a and b are, the same or different, 0 to 3, and are not 0 at the same time, L³ is a single bond, R³ is alkyl having 1 to 6 carbon atoms, alkenyl having 3 to 6 carbon atoms, pyrrolidin-3-yl, piperidin-3-yl, piperidin-4-yl, or alkyl having 1 to 6 carbon atoms or alkenyl having 3 to 6 carbon atoms substituted with 1 to 3 substituent(s), which is(are), the same or different, amino, monoalkylamino, dialkylamino, trialkylammonio, hydroxy, alkoxy, carbamoyl, monoalkylcarbamoyl, dialkylcarbamoyl, pyrrolidinyl, piperidyl or morpholinyl, L¹ and L² are, the same or different, —O—, —CO—O or O—CO,
Y is absent, a and b are, the same or different, 0 to 3, L³ is a single bond, R³ is a hydrogen atom, and L¹ and L² are, the same or different, —O—, CO—O or O—CO—, or
Y is absent, a and b are, the same or different, 0 to 3, L³ is CO or CO—O—, R³is pyrrolidin-2-yl, pyrrolidin-3-yl, piperidin-2-yl, piperidin-3-yl, piperidin-4-yl, morpholin-2-yl, morpholin-3-yl, or alkyl having 1 to 6 carbon atoms or alkenyl having 3 to 6 carbon atoms substituted with 1 to 3 substituent(s), which is(are), the same or different, amino, monoalkylamino, dialkylamino, trialkylammonio, hydroxy, alkoxy, carbamoyl, monoalkylcarbamoyl, dialkylcarbamoyl, pyrrolidinyl, piperidyl or morpholinyl, wherein at least one of the substituents is amino, monoalkylamino, dialkylamino, trialkylammonio, pyrrolidinyl, piperidyl or morpholinyl, and L¹ and L² are, the same or different, —O—, CO—O or O—CO—, and
when X³ is alkyl having 1 to 6 carbon atoms or alkenyl having 3 to 6 carbon atoms,
Y is a pharmaceutically acceptable anion, a and b are, the same or different, 0 to 3, L³ is a single bond, R³ is alkyl having 1 to 6 carbon atoms, alkenyl having 3 to 6 carbon atoms, pyrrolidin-2-yl, pyrrolidin-3-yl, piperidin-2-yl, piperidin-3-yl, piperidin-4-yl, morpholin-2-yl, morpholin-3-yl, or alkyl having 1 to 6 carbon atoms or alkenyl having 3 to 6 carbon atoms substituted with 1 to 3 substituent(s), which is(are), the same or different, amino, monoalkylamino, dialkylamino, trialkylammonio, hydroxy, alkoxy, carbamoyl, monoalkylcarbamoyl, dialkylcarbamoyl, pyrrolidinyl, piperidyl or morpholinyl, L¹ and L² are, the same or different, —O—, —CO—O— or —O—CO—). In some embodiments, an LNP described herein comprises a lipid, e.g., an ionizable lipid, disclosed in US 2013/0195920, which is incorporated herein by reference in its entirety.

In some embodiments, the lipids disclosed in US 2013/0195920 are of formula (I), which has a branched alkyl at the alpha position adjacent to the biodegradable group (between the biodegradable group and the teriary carbon):

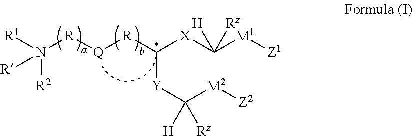

or a salt thereof (e.g., a pharmaceutically acceptable salt thereof), wherein
R′ is absent, hydrogen, or alkyl (e.g., C₁-C₄ alkyl);
with respect to R¹ and R²,
(i) R¹ and R² are each, independently, optionally substituted alkyl, alkenyl, alkynyl, cycloalkylalkyl, heterocycle, or R¹⁰;
(ii) R¹ and R², together with the nitrogen atom to which they are attached, form an optionally substituted heterocylic ring; or
(iii) one of R¹ and R² is optionally substituted alkyl, alkenyl, alkynyl, cycloalkyl, cycloalkylalkyl, or heterocycle, and the other forms a 4-10 member heterocyclic ring or heteroaryl (e.g., a 6-member ring) with (a) the adjacent nitrogen atom and (b) the (R)ₐgroup adjacent to the nitrogen atom;
each occurrence of R is, independently, —(CR³R⁴)—;
each occurrence of R³ and R⁴ are, independently H, halogen, OH, alkyl, alkoxy, —NH₂, R¹⁰, alkylamino, or dialkylamino (In some embodiments, each occurrence of R³ and R⁴ are, independently H or C₁-C₄ alkyl);
each occurrence of R¹⁰ is independently selected from PEG and polymers based on poly(oxazoline), poly(ethylene oxide), poly(vinyl alcohol), poly(glycerol), poly(N-vinylpyrrolidone), poly[N-(2-hydroxypropyl)methacrylamide] and poly(amino acid)s, wherein (i) the PEG or polymer is linear or branched, (ii) the PEG or polymer is polymerized by n subunits, (iii) n is a number-averaged degree of polymerization between 10 and 200 units, and (iv) wherein the compound of formula has at most two R¹⁰ groups (preferably at most one R¹⁰ group);
the dashed line to Q is absent or a bond;
when the dashed line to Q is absent then Q is absent or is —O—, —NH—, —S—, C(O), —C(O)O—, —OC(O)—, C(O)N(R⁴)—, —N(R⁵)C(O)—, —S—S—, OC(O)O—, —O—N═C(R⁵)—, —C(R⁵)═N—O—, OC(O)N(R⁵)—, —N(R⁵)C(O)N(R⁵)—, —N(R⁵)C(O)O—, —C(O)S—, C(S)O or C(R⁵)═N—O—C(O)—; or
when the dashed line to Q is a bond then (i) b is 0 and (ii) Q and the tertiary carbon adjacent to it (C*) form a substituted or unsubstituted, mono- or bi-cyclic heterocyclic group having from 5 to 10 ring atoms (e.g., the heteroatoms in the heterocyclic group are selected from O and S, preferably 0);
each occurrence of R⁵ is, independently, H or alkyl (e.g. C₁-C₄ alkyl);
X and Y are each, independently, alkylene or alkenylene (e.g., C₄to C₂Oalkylene or C₄to C₂Oalkenylene);
M¹ and M² are each, independently, a biodegradable group (e.g., —OC(O)—, —C(O)O—, —SC(O)—, C(O)S—, —OC(S)—, —C(S)O—, —S—S—, C(R⁵)═N—, —N═C(R⁵)—, —C(R⁵)═N—O—, —O—N═C(R⁵)—, —C(O)(NR⁵)—, N(R⁵)C(O)—, C(S)(NR⁵)—, N(R⁵)C(O)—, —N(R⁵)C(O)N(R⁵)—, OC(O)O—, OSi(R⁵)₂O—, C(O)(CR³R⁴)C(O)O—, —OC(O)(CR³R⁴)C(O)—, or

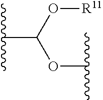

wherein R¹¹ is a C₂-C₈ alkyl or alkenyl;
each occurrence of R^{z} is, independently, C₁-C₈ alkyl (e.g., methyl, ethyl, isopropyl, n-butyl, n-pentyl, or n-hexyl);
a is 1, 2, 3, 4, 5 or 6;
b is 0, 1, 2, or 3; and
Z¹ and Z² are each, independently, C₈-C₁₄ alkyl or C₈-C₁₄ alkenyl, wherein the alkenyl group may optionally be substituted with one or two fluorine atoms at the alpha position to a double bond which is between the double bond and the terminus of Z¹ or Z².

In some embodiments, the lipids disclosed in US 2013/0195920 are of formula (II), which has a branched alkyl at the alpha position adjacent to the biodegradable group (between the biodegradable group and the terminus of the tail, i.e., Z¹ or Z²)

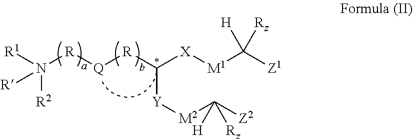

or a salt thereof (e.g., a pharmaceutically acceptable salt thereof), wherein
R′ is absent, hydrogen, or alkyl (e.g., C₁-C₄ alkyl);
with respect to R¹ and R²,
(i) R¹ and R² are each, independently, optionally substituted alkyl, alkenyl, alkynyl, cycloalkylalkyl, heterocycle, or R¹⁰;
(ii) R¹ and R², together with the nitrogen atom to which they are attached, form an optionally substituted heterocylic ring; or
(iii) one of R¹ and R² is optionally substituted alkyl, alkenyl, alkynyl, cycloalkyl, cycloalkylalkyl, or heterocycle, and the other forms a 4-10 membered heterocyclic ring or heteroaryl (e.g., a 6-member ring) with (a) the adjacent nitrogen atom and (b) the (R)ₐgroup adjacent to the nitrogen atom;
each occurrence of R is, independently, —(CR³R⁴)—;
each occurrence of R³ and R⁴ are, independently H, halogen, OH, alkyl, alkoxy, NH2, R¹⁰, alkylamino, or dialkylamino (In some embodiments, each occurrence of R³ and R⁴ are, independently H or C₁-C₄ alkyl);
each occurrence of R¹⁰ is independently selected from PEG and polymers based on poly(oxazoline), poly(ethylene oxide), poly(vinyl alcohol), poly(glycerol), poly(N-vinylpyrrolidone), poly[N-(2-hydroxypropyl)methacrylamide] and poly(amino acid)s, wherein (i) the PEG or polymer is linear or branched, (ii) the PEG or polymer is polymerized by n subunits, (iii) n is a number-averaged degree of polymerization between 10 and 200 units, and (iv) wherein the compound of formula has at most two R¹⁰ groups (preferably at most one R¹⁰ group);
the dashed line to Q is absent or a bond;
when the dashed line to Q is absent then Q is absent or is —O—, —NH—, —S—, C(O), —C(O)O—, —OC(O)—, C(O)N(R⁴)—, —N(R⁵)C(O)—, —S—S—, OC(O)O—, —O—N═C(R⁵)—, —C(R⁵)═N—O—, OC(O)N(R⁵)—, —N(R⁵)C(O)N(R⁵)—, —N(R⁵)C(O)O—, —C(O)S—, C(S)O or C(R⁵)═N—O—C(O)—; or
when the dashed line to Q is a bond then (i) b is 0 and (ii) Q and the tertiary carbon adjacent to it (C*) form a substituted or unsubstituted, mono- or bi-cyclic heterocyclic group having from 5 to 10 ring atoms (e.g., the heteroatoms in the heterocyclic group are selected from O and S, preferably 0);
each occurrence of R⁵ is, independently, H or alkyl;
X and Y are each, independently, alkylene (e.g., C₆-C₈ alkylene) or alkenylene, wherein the alkylene or alkenylene group is optionally substituted with one or two fluorine atoms at the alpha position to the M¹ or M2 group
M¹ and M² are each, independently, a biodegradable group (e.g., —OC(O)—, —C(O)O—, —SC(O)—, C(O)S—, —OC(S)—, —C(S)O—, —S—S—, C(R⁵)═N—, —N═C(R⁵)—, —C(R⁵)═N—O—, —O—N═C(R⁵)—, —C(O)(NR⁵)—, N(R⁵)C(O)—, C(S)(NR⁵)—, N(R⁵)C(O)—, —N(R⁵)C(O)N(R⁵)—, OC(O)O—, OSi(R⁵)₂O—, C(O)(CR³R⁴)C(O)O—, —OC(O)(CR³R⁴)C(O)—, or

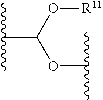

wherein R″ is a C₂-C₈ alkyl or alkenyl;
each occurrence of R^{z} is, independently, C₁-C₈ alkyl (e.g., methyl, ethyl, isopropyl);
a is 1, 2, 3, 4, 5 or 6;
b is 0, 1, 2, or 3; and
Z¹ and Z² are each, independently, C₈-C₁₄ alkyl or C₈-C₁₄ alkenyl, wherein (i) the alkenyl group may optionally be substituted with one or two fluorine atoms at the alpha position to a double bond which is between the double bond and the terminus of Z¹ or Z²;
and (ii) the terminus of at least one of Z¹ and Z² is separated from the group M¹ or M²by at least 8 carbon atoms.

In some embodiments, the lipids disclosed in US 2013/0195920 are of formula (III), which has a branching point at a position that is 2-6 carbon atoms (i.e., at the beta (β), gamma (γ), delta (δ), epsilon (ε) or zeta position (ζ) adjacent to the biodegradable group (between the biodegradable group and the terminus of the tail, i.e., Z¹ or Z²)

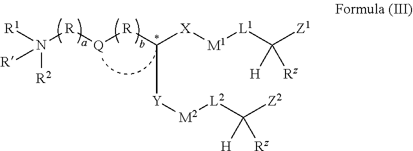

or a salt thereof (e.g., a pharmaceutically acceptable salt thereof), wherein
R′, R¹, R², R, R³, R⁴, R¹⁰, Q, R⁵, M¹, M², R^{z}, a, and b are defined as in formula (I);
L¹ and L² are each, independently, C₁-C₅ alkylene or C₂-C₅ alkenylene;
X and Y are each, independently, alkylene (e.g., C₄to C₂Oalkylene or C₆-C₈ alkylene) or alkenylene (e.g., C₄ to C₂Oalkenylene); and
Z¹ and Z² are each, independently, C₈-C₁₄ alkyl or C₈-C₁₄ alkenyl, wherein the alkenyl group may optionally be substituted with one or two fluorine atoms at the alpha position to a double bond which is between the double bond and the terminus of Z¹ or Z².
and with the proviso that the terminus of at least one of Z¹ and Z² is separated from the group M¹ or M² by at least 8 carbon atoms.

In some embodiments, the cationic lipid disclosed in US 2013/0195920 is a compound of formula (IV), which has a branching point at a position that is 2-6 carbon atoms (i.e., at beta (β), gamma (γ), delta (δ), epsilon (ε) or zeta position (ζ) adjacent to the biodegradable group (between the biodegradable group and the terminus of the tail, i.e., Z¹ or Z²)

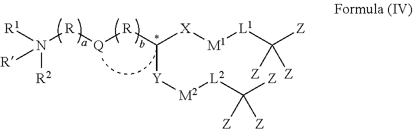

or a salt thereof (e.g., a pharmaceutically acceptable salt thereof), wherein
R′, R¹, R², R, R³, R⁴, R¹⁰, Q, R⁵, M², R^{z}, a, and b are defined as in formula (I);
L¹ and L² and are each, independently, C₁-C₅ alkylene or C₂-C₅ alkenylene;
X and Y are each, independently, alkylene or alkenylene (e.g., C₁₂-C₂Oalkylene or C₁₂-C₂Oalkenylene); and
each occurrence of Z is independently C₁-C₄ alkyl (preferably, methyl).

For example, in some embodiments, -L¹-C(Z)₃ is CH₂C(CH₃)₃. In some embodiments, -L¹-C(Z)₃ is CH₂CH₂C(CH₃)₃.

In some embodiments, the lipids disclosed in US 2013/0195920 are of formula (V), which has an alkoxy or thioalkoxy (i.e., —S-alkyl) group substitution on at least one tail:

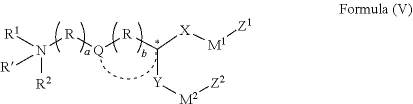

or a salt thereof (e.g., a pharmaceutically acceptable salt thereof), wherein
R′, R¹, R², R, R³, R⁴, R¹⁰, Q, R⁵, M¹, M², a, and b are defined as in formula (I);
X and Y are each, independently, alkylene (e.g., C₆-C₈ alkylene) or alkenylene, wherein the alkylene or alkenylene group is optionally substituted with one or two fluorine atoms at the alpha position to the M¹ or M² group;
Z¹ and Z² are each, independently, C₈-C₁₄ alkyl or C₈-C₁₄ alkenyl, wherein (i) the C₈-C₁₄ alkyl or C₈-C₁₄ alkenyl of at least one of Z¹ and Z² is substituted by one or more alkoxy (e.g., a C₁-C₄ alkoxy such as —OCH₃) or thioalkoxy (e.g., a C₁-C₄ thioalkoxy such as —SCH₃) groups, and (ii) the alkenyl group may optionally be substituted with one or two fluorine atoms at the alpha position to a double bond which is between the double bond and the terminus of Z¹ or Z².

In some embodiments, the lipids disclosed in US 2013/0195920 are of formula (VIA), which has one or more fluoro substituents on at least one tail at a position that is either alpha to a double bond or alpha to a biodegradable group:

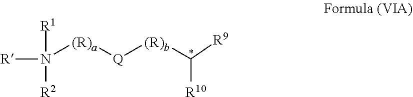

or a salt thereof (e.g., a pharmaceutically acceptable salt thereof), wherein
R¹, R², R, a, and b are as defined with respect to formula (I);
Q is absent or is —O—, —NH—, —S—, —C(O)—, —C(O)O—, —OC(O)—, —C(O)N(R⁴)—, —N(R⁵)C(O)—, —S—S—, OC(O)O—, —O—N═C(R⁵)—, —C(R⁵)═N—O, —OC(O)N(R⁵)—, —N(R⁵)C(O)N(R⁵)—, —N(R⁵)C(O)O—, C(O)S—, C(S)O or —C(R⁵)═N—O—C(O)—;
R′ is absent, hydrogen, or alkyl (e.g., C₁-C₄ alkyl); and
each of R⁹ and R¹⁰ are independently C₁₂-C₂₄ alkyl (e.g., C₁₂-C₂Oalkyl), C₁₂-C₂₄ alkenyl (e.g., C₁₂-C₂Oalkenyl), or C₁₂-C₂₄ alkoxy (e.g., C₁₂-C₂Oalkoxy) (a) having one or more biodegradable groups and (b) optionally substituted with one or more fluorine atoms at a position which is (i) alpha to a biodegradable group and between the biodegradable group and the tertiary carbon atom marked with an asterisk (*), or (ii) alpha to a carbon-carbon double bond and between the double bond and the terminus of the R⁹ or R¹⁰ group; each biodegradable group independently interrupts the C₁₂-C₂₄ alkyl, alkenyl, or alkoxy group or is substituted at the terminus of the C₁₂-C₂₄ alkyl, alkenyl, or alkoxy group, wherein
(i) at least one of R⁹ and R¹⁰ contains a fluoro group;
(ii) the compound does not contain the following moiety:

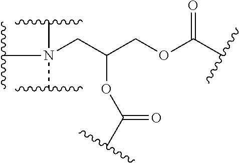

wherein — — — — is an optional bond; and
(iii) the terminus of R⁹ and R¹⁰ is separated from the tertiary carbon atom marked with an asterisk (*) by a chain of 8 or more atoms (e.g., 12 or 14 or more atoms).
   In some embodiments, the terminus of R⁹ and R¹⁰ is separated from the tertiary carbon atom marked with an asterisk (*) by a chain of 18-22 carbon atoms (e.g., 18-20 carbon atoms).
   In some embodiments, the lipids disclosed in US 2013/0195920 are of formula (VIB), which has one or more fluoro substituents on at least one tail at a position that is either alpha to a double bond or alpha to a biodegradable group.

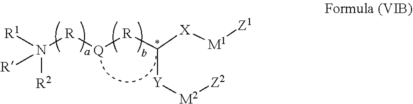

or a salt thereof (e.g., a pharmaceutically acceptable salt thereof), wherein
R′, R¹, R², R, R³, R⁴, R¹⁰, Q, R⁵, M¹, M², a, and b are defined as in formula (I);
X and Y are each, independently, alkylene (e.g., C₆-C₈ alkylene) or alkenylene, wherein the alkylene or alkenylene group is optionally substituted with one or two fluorine atoms at the alpha position to the M¹ or M² group; and
Z¹ and Z² are each, independently, C₈-C₁₄ alkyl or C₈-C₁₄ alkenyl, wherein said C₅-C₁₄ alkenyl is optionally substituted by one or more fluorine atoms at a position that is alpha to a double bond,
wherein at least one of X, Y, Z¹, and Z² contains a fluorine atom.

In some embodiments, the lipids disclosed in US 2013/0195920 are of formula (VII), which has an acetal group as a biodegradable group in at least one tail:

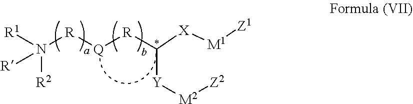

or a salt thereof (e.g., a pharmaceutically acceptable salt thereof), wherein
R′, R¹, R², R, R³, R⁴, R¹⁰, Q, R⁵, a, and b are defined as in formula (I);
X and Y are each, independently, alkylene (e.g., C₆-C₈ alkylene) or alkenylene, wherein the alkylene or alkenylene group is optionally substituted with one or two fluorine atoms at the alpha position to the M¹ or M² group
M¹ and M² are each, independently, a biodegradable group (e.g., —OC(O)—, C(O)O—, —SC(O)—, C(O)S—, —OC(S)—, —C(S)O—, —S—S—, C(R⁵)═N—, —N═C(R⁵)—, —C(R⁵)═N—O—, —O—N═C(R⁵)—, —C(O)(NR⁵)—, N(R⁵)C(O)—, C(S)(NR⁵)—, N(R⁵)C(O)—, —N(R⁵)C(O)N(R⁵)—, OC(O)O—, OSi(R⁵)₂O—, C(O)(CR³R⁴)C(O)O—, —OC(O)(CR³R⁴)C(O)—, or

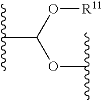

wherein R″ is a C₄-C₁₀ alkyl or C₄-C₁₀ alkenyl;
with the proviso that at least one of M¹ and M² is

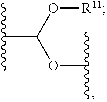

and
Z¹ and Z² are each, independently, C₄-C₁₄ alkyl or C₄-C₁₄ alkenyl, wherein the alkenyl group may optionally be substituted with one or two fluorine atoms at the alpha position to a double bond which is between the double bond and the terminus of Z¹ or Z².

In some embodiments, an LNP described herein comprises a lipid, e.g., an ionizable lipid, disclosed in US 2015/0005363, which is incorporated herein by reference in its entirety.

In some embodiments, an LNP described herein comprises a lipid, e.g., an ionizable lipid, disclosed in US 2014/0308304, which is incorporated herein by reference in its entirety. In some embodiments, the lipid disclosed in US 2014/0308304 is a compound of formula (I):

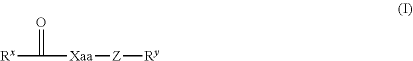

or a salt thereof (e.g., a pharmaceutically acceptable salt thereof), wherein
Xaa is a D- or L-amino acid residue having the formula —NR^{N}—CR¹R² (C═O), or a peptide of amino acid residues having the formula {NR^{N} CR¹R² (C═O)}ₙ, wherein n is 2 to 20;
R¹ is independently, for each occurrence, a non-hydrogen, substituted or unsubstituted side chain of an amino acid;
R² and R^{N} are independently, for each occurrence, hydrogen, an organic group consisting of carbon, oxygen, nitrogen, sulfur, and hydrogen atoms, or any combination of the foregoing, and having from 1 to 20 carbon atoms, C₍₁₋₅)alkyl, cycloalkyl, cycloalkylalkyl, C₍₃-5)alkenyl, C₍₃-5)alkynyl, C₍₁-5)alkanoyl, C₍₁₋₅)alkanoyloxy, C₍₁₋₅)alkoxy, C₍₁₋₅)alkoxy-C₍₁₋₅)alkyl, C₍₁₋₅)alkoxy-C₍₁₋₅)alkoxy, C₍₁-5)alkyl-amino-C₍₁₋₅)alkyl-, C₍₁₋₅)dialkyl-amino-C₍₁₋₅)alkyl-, nitro-C₍₁—S)alkyl, cyano-C₍₁—S)alkyl, aryl-C₍₁-5)alkyl, 4-biphenyl-C₍₁₋₅)alkyl, carboxyl, or hydroxyl;
Z is NH, O, —S—, —CH₂S—, CH₂S(O)—, or an organic linker consisting of 1-40 atoms selected from hydrogen, carbon, oxygen, nitrogen, and sulfur atoms (preferably, Z is NH or O);
R^{x} and R^{y} are, independently, (i) a lipophilic tail derived from a lipid (which can be naturally-occurring or synthetic), phospholipid, glycolipid, triacylglycerol, glycerophospholipid, sphingolipid, ceramide, sphingomyelin, cerebroside, or ganglioside, wherein the tail optionally includes a steroid; (ii) an amino acid terminal group selected from hydrogen, hydroxyl, amino, and an organic protecting group; or (iii) a substituted or unsubstituted C₍₃-22)alkyl, C₍₆-12)cycloalkyl, C₍₆-12)cycloalkyl-C₍₃-22)alkyl, C₍₃-22)alkenyl, C₍₃-22)alkynyl, C₍₃-22)alkoxy, or C₍₆-12)-alkoxy-C₍₃-22)alkyl;
one of Rand R is a lipophilic tail as defined above and the other is an amino acid terminal group, or both R^{x} and R^{y} are lipophilic tails;
at least one of R^{x} and R^{y} is interrupted by one or more biodegradable groups (e.g., OC(O) C(O)O—, SC(O)—, C(O)S—, —OC(S)—, C(S)O—, —S—S—, C(R⁵)═N—N═C(R⁵)—, —C(R⁵)═N—O—, —O—N═C(R⁵)—, —C(O)(NR⁵)—, N(R⁵)C(O)—, —C(S)(NR⁵)—, N(R⁵)C(O)—, —N(R⁵)C(O)N(R⁵)—, OC(O)O—, OSi(R⁵)₂O, —C(O)(CR³R⁴)C(O)O—, OC(O)(CR R⁴)C(O)— or

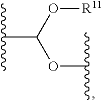

(wherein R¹¹ is a C₂-C₈ alkyl or alkenyl), in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in some embodiments, each occurrence of R³ and R⁴ are, independently H or C₁-C₄ alkyl)); and
R^{x} and R^{y} each, independently, optionally have one or more carbon-carbon double bonds. In some embodiments the lipid disclosed in US 2014/0308304 is a compound of formula (IA):

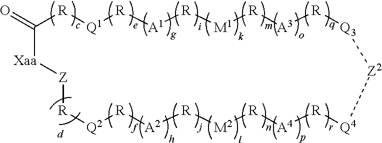

or a salt thereof (e.g., a pharmaceutically acceptable salt thereof), wherein
Z and Xaa are as defined with respect to formula (I) (the variables which are used in the definition of Xaa, namely R^{N}, R¹ and R², are also as defined in formula (I));
each occurrence of R is, independently, —(CR³R⁴);
each occurrence of R³ and R⁴ are, independently H, halogen, OH, alkyl, alkoxy, —NH₂, alkylamino, or dialkylamino (in some embodiments, each occurrence of R³ and R⁴ are, independently H or C₁-C₄ alkyl);
or R³ and R⁴, together with the carbon atom to which they are directly attached, form a cycloalkyl group, wherein no more than three R groups in each chain between the Z-Xaa-C(O) and Z² moieties are cycloalkyl (e.g., cyclopropyl);
Q¹ and Q²are each, independently, absent, —O—, —S—, —OC(O)—, C(O)O—, —SC(O)—, C(O)S—, —OC(S)—, C(S)O—, —S—S—, C(O)(NR⁵)—, N(R⁵)C(O)—, —C(S)(NR⁵)—, N(R⁵)C(O)—, —N(R⁵)C(O)N(R⁵)—, or OC(O)O—;
Q³ and Q⁴ are each, independently, H, —(CR³R⁴)—, cycloalkyl, heterocyclyl, heterocyclylalkyl, aryl, heteroaryl, or a cholesterol moiety;
each occurrence of A1, A², A³ and A⁴is, independently, —(CR⁵R5-CR⁵═CR⁵)—;
M¹ and M² are each, independently, a biodegradable group (e.g., —OC(O)—, C(O)O—, —SC(O)—, C(O)S—, —OC(S)—, C(S)O—, —S—S—, C(R⁵)═N—, —N═C(R⁵) C(R⁵)═N—O—, —O—N═C(R⁵)—, —C(O)(NR⁵)—, N(R⁵)C(O)—, C(S)(NR⁵), N(R⁵)C(O)—, —N(R⁵)C(O)N(R⁵)—, OC(O)O—, OSi(R⁵)₂O—, C(O)(CR³R⁴)C(O)O—, —OC(O)(CR³R⁴)C(O)—, or

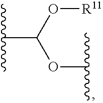

(wherein R¹¹ is a C₂-C₈ alkyl or alkenyl));
each occurrence of R⁵ is, independently, H or alkyl (e.g., C₁-C₄ alkyl);
Z² is absent, alkylene or O P(O)(OH)—O—;
each ————— attached to Z² is an optional bond, such that when Z² is absent, Q³ and Q⁴ are not directly covalently bound together;
c, d, e, f, i, j, m, n, q and r are each, independently, —O—, 1, 2, 3, 4, 5, 6, 7, 8, 9, or 10;
g and h are each, independently, —O—, 1 or 2;
k and 1 are each, independently, 0 or 1, wherein at least one of k and 1 is 1;
o and p are each, independently, —O—, 1 or 2; and
Q³ and Q⁴ are each, independently, separated from the Z-Xaa-C(O) moiety by a chain of 8 or more atoms (e.g., 12 or 14 or more atoms).

In some embodiments the lipids disclosed in US 2014/0308304 are of the formula (IC):

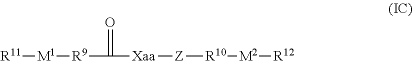

or a salt thereof (e.g., a pharmaceutically acceptable salt thereof), wherein
Z and Xaa are as defined with respect to formula (I) (the variables which are used in the definition of Xaa, namely R^{N}, R¹ and R², are also as defined in formula (I));
each of R⁹ and R¹⁰ are, independently, alkylene or alkenylene;
each of R¹¹ and R¹² are, independently, alkyl or alkenyl, optionally terminated by COOR¹³ wherein each R¹³ is independently unsubstituted alkyl (e.g., C₁-C₄ alkyl such as methyl or ethyl), substituted alkyl (such as benzyl), or cycloalkyl;
M¹ and M² are each, independently, a biodegradable group (e.g., —OC(O)—, —C(O)O—, —SC(O)—, C(O)S—, —OC(S)—, C(S)O—, —S—S—, C(R⁵)═N—, —N═C(R⁵) C(R⁵)═N—O—, —O—N═C(R⁵)—, —C(O)(NR⁵)—, N(R⁵)C(O)—, C(S)(NR⁵), N(R⁵)C(O)—, —N(R⁵)C(O)N(R⁵)—, OC(O)O—, OSi(R⁵)₂O—, C(O)(CR³R⁴)C(O)O—, —OC(O)(CR³R⁴)C(O)—, or

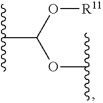

wherein R¹¹ is a C₂-C₈ alkyl or alkenyl, in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in some embodiments, each occurrence of R³ and R⁴ are, independently H or C₁-C₄ alkyl));
R⁹, M¹, and R¹¹ are together at least 8 carbon atoms in length (e.g., 12 or 14 carbon atoms or longer); and
R¹⁰, M², and R¹² are together at least 8 carbon atoms in length (e.g., 12 or 14 carbon atoms or longer).
   In some embodiments, the lipid disclosed in US 2014/0308304 is a compound of the formula II:

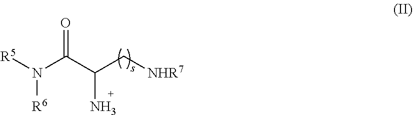

or a salt thereof (e.g., a pharmaceutically acceptable salt thereof), wherein:
s is 1, 2, 3 or 4; and
R⁷ is selected from lysyl, ornithyl, 2,3-diaminobutyryl, histidyl and an acyl moiety of the formula:

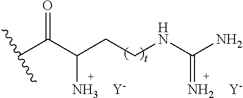

t is 1, 2 or 3;
the NH₃+ moiety in the acyl moiety in R⁷ is optionally absent;
each occurrence of Y⁻is independently a pharmaceutically acceptable anion (e.g., halide, such
as chloride);
R⁵ and R⁶ are each, independently a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid; or a substituted or unsubstituted C₍₃-22)alkyl, C₍₆-12)cycloalkyl, C₍₆-12)cycloalkyl-C₍₃-22)alkyl, C₍₃-22)alkenyl, C₍₃-22)alkynyl, C₍₃-22)alkoxy, or C₍₆-12)alkoxy-C₍₃-22)alkyl;
at least one of R⁵ and R⁶ is interrupted by one or more biodegradable groups (e.g., —SC(O)—, —C(O)S—, —OC(S)—, —C(S)O—, —S—S—, —C(O)(NR^{a})—, —N(R^{a})C(O)—, —C(S)(NR^{a})—, —N(R^{a})C(O)—, N(R^{a})C(O)N(R^{a})—, or OC(O)O—);
each occurrence of R^{a} is, independently, H or alkyl; and
R⁵ and R⁶ each, independently, optionally contain one or more carbon-carbon double bonds.
   In some embodiments, the lipids disclosed in US 2014/0308304 are of the formula (IIA):

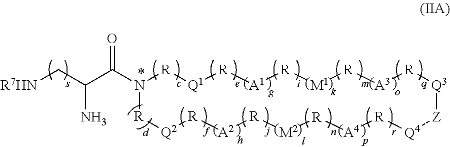

or a salt thereof (e.g., a pharmaceutically acceptable salt thereof), wherein:
R⁷ and s are as defined with respect to formula (II);
each occurrence of R is, independently, —(CR³R⁴)—;
each occurrence of R³ and R⁴ are, independently H, halogen, OH, alkyl, alkoxy, —NH₂, alkylamino, or dialkylamino (in some embodiments, each occurrence of R³ and R⁴ are, independently H or C₁-C₄ alkyl);
or R³ and R⁴, together with the carbon atom to which they are directly attached, form a cycloalkyl group, wherein no more than three R groups in each chain attached to the nitrogen N* are cycloalkyl (e.g., cyclopropyl);
Q¹ and Q²are each, independently, absent, —O—, —S—, —OC(O)—, C(O)O—, —SC(O)—, C(O)S—, —OC(S)—, —C(S)O—, —S—S—, —C(O)(NR⁵), —N(R⁵)C(O)—, C(S)(NR⁵)—, N(R⁵)C(O)—, —N(R⁵)C(O)N(R⁵)—, or OC(O)O—;
Q³ and Q⁴ are each, independently, H, —(CR³R⁴)—, aryl, cycloalkyl, heterocyclyl, heterocyclylalkyl, heteroaryl, or a cholesterol moiety;
each occurrence of A¹, A², A³ and A⁴ is, independently, —(CR⁵R⁵CR⁵═CR⁵)—;
M¹ and M² are each, independently, a biodegradable group (e.g., —OC(O)—, —C(O)O—, —SC(O)—, C(O)S—, —OC(S)—, C(S)O—, —S—S—, C(R⁵)═N—, —N═C(R⁵) C(R⁵)═N—O—, —O—N═C(R⁵)—, —C(O)(NR⁵)—, N(R⁵)C(O)—, C(S)(NR⁵), N(R⁵)C(O)—, —N(R⁵)C(O)N(R⁵)—, OC(O)O—, OSi(R⁵)₂O—, C(O)(CR³R⁴)C(O)O—, —OC(O)(CR³R⁴)C(O)—, or

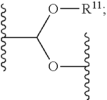

wherein R¹¹ is a C₂-C₈ alkyl or alkenyl;
each occurrence of R⁵ is, independently, H or alkyl;
Z is absent, alkylene or O P(O)(OH)—O—;
each ————— attached to Z is an optional bond, such that when Z is absent, Q³ and Q⁴ are not directly covalently bound together;
c, d, e, f, i, j, m, n, q and r are each, independently, —O—, 1, 2, 3, 4, 5, 6, 7, 8, 9, or 10;
g and h are each, independently, —O—, 1 or 2;
k and 1 are each, independently, 0 or 1, where at least one of k and 1 is 1; and
o and p are each, independently, —O—, 1 or 2.

In some embodiments the lipid disclosed in US 2014/0308304 are of the formula (IIC):

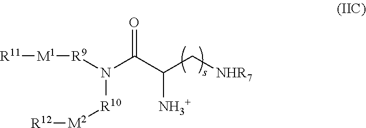

or a salt thereof (e.g., a pharmaceutically acceptable salt thereof), wherein:
R⁷ and s are as defined with respect to formula (II);
each of R⁹ and R¹⁰ are independently alkyl (e.g., C₁₂-C₂₄ alkyl) or alkenyl (e.g., C₁₂-C₂₄ alkenyl);
each of R¹¹ and R¹² are independently alkyl or alkenyl, optionally terminated by COOR¹³ where each R¹³ is independently alkyl (e.g., C₁-C₄ alkyl such as methyl or ethyl);
M¹ and M² are each, independently, a biodegradable group (e.g., —OC(O)—, —C(O)O—, —SC(O)—, C(O)S—, —OC(S)—, C(S)O—, —S—S—, C(R⁵)═N—, —N═C(R⁵) C(R⁵)═N—O—, —O—N═C(R⁵)—, —C(O)(NR⁵)—, N(R⁵)C(O)—, C(S)(NR⁵), N(R⁵)C(O)—, —N(R⁵)C(O)N(R⁵)—, OC(O)O—, OSi(R⁵)₂O—, C(O)(CR³R⁴)C(O)O—, —OC(O)(CR³R⁴)C(O)—, or

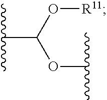

wherein R¹¹ is a C₂-C₈ alkyl or alkenyl;
in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in some embodiments, each occurrence of R³ and R⁴ are, independently, H or C₁-C₄ alkyl));
R⁹, M¹, and R¹¹ are together at least 8 carbons atoms in length (e.g., 12 or 14 carbon atoms or longer); and
R¹⁰, M², and R¹² are together at least 8 carbons atoms in length (e.g., 12 or 14 carbon atoms or longer).

In some embodiments, the lipid disclosed in US 2014/0308304 is a compound of the formula (4):

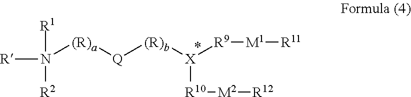

wherein:
X is N or P;
R¹, R², R, a, b, M¹, and M² are as defined with respect to formula (I);
Q is absent or is —O—, —NH—, —S, C(O)O—, —OC(O)—, C(O)N(R⁴), N(R⁵)C(O)—, —S—S—, OC(O)O—, —O—N═C(R⁵)—, —C(R⁵)═N—O—, OC(O)N(R⁵) N(R⁵)C(O)N(R⁵)—, —N(R⁵)C(O)O—, C(O)S—, C(S)O or C(R⁵)═N—O—C(O);
R′ is absent, hydrogen, or alkyl (e.g., C₁-C₄ alkyl);
each of R⁹ and R¹⁰ are independently alkylene, or alkenylene; and
each of R¹¹ and R¹² are independently alkyl or alkenyl, optionally terminated by COOR¹³ where each R¹³ is independently alkyl (e.g., C₁-C₄ alkyl such as methyl or ethyl);
R⁹, M¹, and R¹¹ are together at least 8 carbons atoms in length (e.g., 12 or 14 carbon atoms or longer); and
R¹⁰, M², and R¹² are together at least 8 carbons atoms in length (e.g., 12 or 14 carbon atoms or longer).

In some embodiments, the lipid disclosed in US 2014/0308304 is a compound of the formula (5)

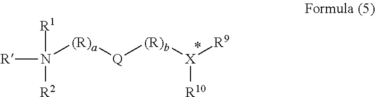

wherein:
X is N or P;
R¹, R², R, a, and b are as defined with respect to formula (I);
Q is absent or is —O—, —NH—, —S, C(O)O—, —OC(O)—, C(O)N(R⁴), N(R⁵)C(O)—, —S—S—, OC(O)O—, —O—N═C(R⁵)—, —C(R⁵)═N—O—, OC(O)N(R⁵), N(R⁵)C(O)N(R⁵)—, —N(R⁵)C(O)O—, C(O)S—, C(S)O or C(R⁵)═N—O—C(O)—;R′ is absent, hydrogen, or alkyl (e.g., C₁-C₄ alkyl);
each of R⁹ and R¹⁰ are independently C₁₂-C₂₄ alkyl or alkenyl substituted at its terminus with a biodegradable group, such as COOR¹³ where each R¹³ is independently alkyl (preferably C₁-C₄ alkyl such as methyl or ethyl).

In some embodiments the lipids disclosed in US 2014/0308304 are of Formula A:

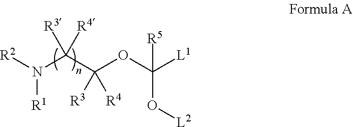

or a pharmaceutically acceptable salt or stereoisomer thereof, wherein:
n is 0-6 (e.g., n is 0, 1 or 2);
R¹ and R² are independently selected from H, (C₁-C₆)alkyl, heterocyclyl, and a polyamine,
wherein said alkyl, heterocyclyl and polyamine are optionally substituted with one or more substituents selected from R′, or R¹ and R² can be taken together with the nitrogen to which they are attached to form a monocyclic heterocycle with 3-7 (e.g., 4-7) members optionally containing, in addition to the nitrogen, one or two additional heteroatoms selected from N, O and S, said monocyclic heterocycle is optionally substituted with one or more substituents selected from R¹;
R³ is selected from H and (C₁-C₆)alkyl, wherein said alkyl is optionally substituted with one or more substituents selected from R′, or R3 can be taken together with R¹ to form a monocyclic heterocycle with 3-7 (e.g., 4-7) members optionally containing, in addition to the nitrogen, one or two additional heteroatoms selected from N, O and S, said monocyclic heterocycle is optionally substituted with one or more substituents selected from R′;
each occurrence of R⁴, R^{3′} and R^{4′} is independently selected from H, (C₁-C₆)alkyl and O-alkyl, said alkyl is optionally substituted with one or more substituents selected from R′; or R^{3′} and R^{4′} when directly bound to the same carbon atom form an oxo (═O) group, cyclopropyl or cyclobutyl;
or R³ and R⁴ form an oxo (═O) group;
R⁵ is selected from H and (C₁-C₆)alkyl; or R⁵ can be taken together with R¹ to form a monocyclic heterocycle with 4-7 members optionally containing, in addition to the nitrogen, one or two additional heteroatoms selected from N, O and S, said monocyclic heterocycle is optionally substituted with one or more substituents selected from R′;
each occurrence of R′ is independently selected from halogen, R″, OR″, SR″, CN, C₀₂R¹¹ and CON(R″)₂;
each occurrence of R¹¹ is selected from H and (C₁-C₆)alkyl, wherein said alkyl is optionally substituted with one or more substituents selected from halogen and OH;
L¹ is a C₄-C₂₂ alkyl or C₄-C₂₂ alkenyl, said alkyl or alkenyl is optionally interrupted by or terminated with one or more biodegradable groups; and said alkyl or alkenyl is optionally substituted with one or more substituents selected from R′; and
L² is a C₄-C₂₂ alkyl or C₄-C₂₂ alkenyl, said alkyl or alkenyl is optionally interrupted by or terminated with one or more biodegradable groups; and said alkyl or alkenyl is optionally substituted with one or more substituents selected from R′;
with the proviso that the CR^{Y}R^{4′} group when present adjacent to the nitrogen atom in formula A is not a ketone (—C(O)—).

In some embodiments the lipids disclosed in US 2014/0308304 are of formula B:

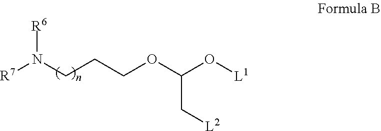

or a pharmaceutically acceptable salt or stereoisomer thereof, wherein:
n is 0, 1, 2, 3, 4, or 5;
R⁶ and R⁷are each independently (i) C₁-C₄linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;
L¹ is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′; and
L² is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′;
each occurrence of R′ is independently selected from halogen, R″, OR″, SR″, CN, CO₂R″ and CON(R″)₂; and
each occurrence of R¹¹ is independently selected from H and (C₁-C₆)alkyl, wherein said alkyl is optionally substituted with one or more substituents selected from halogen and OH.

In some embodiments, lipids disclosed in US 2014/0308304 are of formula C:

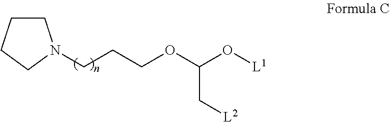

or a pharmaceutically acceptable salt or stereoisomer thereof, wherein:
n is 0, 1, 2, 3, 4, or 5;
L¹ is a C₄-C₂₂ alkyl or C₄-C₂₂ alkenyl, said alkyl or alkenyl optionally has one or more biodegradable groups; each biodegradable group independently interrupts the alkyl or alkenyl group or is substituted at the terminus of the alkyl or alkenyl group, and said alkyl or alkenyl is optionally substituted with 1-5 substituents selected from R′; and
L² is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′;
each occurrence of R′ is independently selected from halogen, R″, OR″, SR″, CN, CO₂R″ and CON(R″)₂; and
each occurrence of R¹¹ is independently selected from H and (C₁-C₆)alkyl, wherein said alkyl is optionally substituted with one or more substituents selected from halogen and OH.
   In some embodiments, the lipid disclosed in US 2014/0308304 are of formula D:

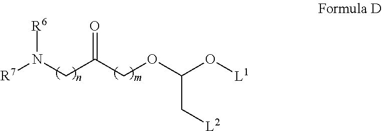

or a pharmaceutically acceptable salt or stereoisomer thereof, wherein
m is 0, 1, 2, or 3;
n is 0, 1, 2, 3, 4, or 5;
R⁶ and R⁷are each independently (i) C₁-C₄linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;
L¹ is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′; and
L² is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′;
each occurrence of R′ is independently selected from halogen, R″, OR″, SR″, CN, CO₂R″ and CON(R″)₂; and
each occurrence of R¹¹ is independently selected from H and (C₁-C₆)alkyl, wherein said alkyl is optionally substituted with one or more substituents selected from halogen and OH.

In some embodiments lipid disclosed in US 2014/0308304 are of formula E:

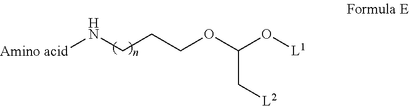

or a pharmaceutically acceptable salt or stereoisomer thereof, wherein
n is 0, 1, 2, 3, 4, or 5;
the group “amino acid” is an amino acid residue;
L¹ is a C₄-C₂₂ alkyl or C₄-C₂₂ alkenyl, said alkyl or alkenyl optionally interrupted by or terminated with one or more biodegradable groups, and said alkyl or alkenyl is optionally substituted with 1-5 substituents selected from R′; and
L² is a C₄-C₂₂ alkyl or C₄-C₂₂ alkenyl, said alkyl or alkenyl optionally interrupted by or terminated with one or more biodegradable groups, and said alkyl or alkenyl is optionally substituted with 1-5 substituents selected from R′;
each occurrence of R′ is independently selected from halogen, R″, OR″, SR″, CN, CO₂R″ and CON(R″)₂; and
each occurrence of R¹¹ is independently selected from H and (C₁-C₆)alkyl, wherein said alkyl is optionally substituted with one or more substituents selected from halogen and OH.

The amino acid residue in formula E may have the formula C(O)—C(R⁹)(NH₂), where R⁹ is an amino acid side chain.

In some embodiments, the lipid disclosed in US 2014/0308304 are of formula F:

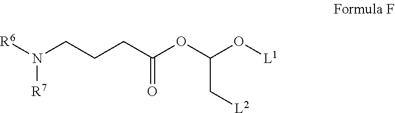

or a pharmaceutically acceptable salt or stereoisomer thereof, wherein:
R⁶ and R⁷ are independently (i) C₁-C₄linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;
L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and
L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;
each occurrence of R′ is independently selected from halogen, R″, OR″, SR″, CN, CO₂R″ and CON(R″)₂;
each occurrence of R¹¹ is independently selected from H and (C₁-C₆)alkyl, wherein said alkyl is optionally substituted with one or more substituents selected from halogen and OH.

In some embodiments, the lipid disclosed in US 2014/0308304 are of formula G:

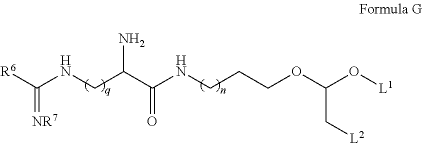

or a pharmaceutically acceptable salt or stereoisomer thereof, wherein:
n is 0, 1, 2, 3, 4, or 5;
q is 1, 2, 3, or 4
R⁶ and R⁷ are independently (i) C₁-C₄linear or branched alkyl (e.g., methyl or ethyl) optionally substituted with 1-4 R′, or (ii) C₃-C₈ cycloalkyl (e.g., C₃-C₆ cycloalkyl);
L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and
L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;
each occurrence of R′ is independently selected from halogen, R″, OR″, SR″, CN, CO₂R″ and CON(R″)₂;
each occurrence of R¹¹ is independently selected from H and (C₁-C₆)alkyl, wherein said alkyl is optionally substituted with one or more substituents selected from halogen and OH.

In some embodiments, an LNP described herein comprises a lipid, e.g., an ionizable lipid, disclosed in US 2013/0053572, which is incorporated herein by reference in its entirety.

In some embodiments, the lipids disclosed in US 2013/0053572 are of Formula A:

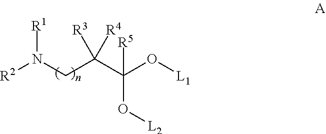

wherein:
n is 0, 1 or 2; R¹ and R² are independently selected from H, (C₁-C₆)alkyl, heterocyclyl, and a polyamine,
wherein said alkyl, heterocyclyl and polyamine are optionally substituted with one or more substituents selected from R′, or R¹, and R² can be taken together with the nitrogen to which they are attached to form a monocyclic heterocycle with 4-7 members optionally containing, in addition to the nitrogen, one or two additional heteroatoms selected from N, O and S, said monocyclic heterocycle is optionally substituted with one or more substituents selected from R¹;
R³ is selected from H and (C₁-C₆)alkyl, wherein said alkyl is optionally substituted with one or more substituents selected from R′, or R3 can be taken together with R1 to form a monocyclic heterocycle with 4-7 members optionally containing, in addition to the nitrogen, one or two additional heteroatoms selected from N, O and S, said monocyclic heterocycle is optionally substituted with one or more substituents selected from R¹;
R⁴ is selected from H, (C₁-C₆)alkyl and O-alkyl, said alkyl is optionally substituted with one or more substituents selected from R¹;
R⁵ is selected from H and (C₁-C₆)alkyl; or R⁵ can be taken together with R¹ to form a monocyclic heterocycle with 4-7 members optionally containing, in addition to the nitrogen, one or two additional heteroatoms selected from N, O and S, said monocyclic heterocycle is optionally substituted with one or more substituents selected from R′;
R′ is independently selected from halogen, R″, OR″, CN, CO₂R″ and CON(R″)₂;
R¹¹ is selected from H and (C₁-C₆)alkyl, wherein said alkyl is optionally substituted with one or more substituents selected from halogen and OH;
L₁ is a C₄-C₂₂ alkenyl, said alkenyl is optionally substituted with one or more substituents selected from R′; and
L₂ is a C₄-C₂₂ alkenyl, said alkenyl is optionally substituted with one or more substituents selected from R′;
or any pharmaceutically acceptable salt or stereoisomer thereof.

In some embodiments, an LNP described herein comprises a lipid, e.g., an ionizable lipid, disclosed in US Application publication US2017/0119904, which is incorporated by reference herein, in its entirety.

In some embodiments, an LNP described herein comprises a lipid, e.g., an ionizable lipid, disclosed in PCT Application publication WO2021/204179, which is incorporated by reference herein, in its entirety.

In some embodiments, an LNP described herein comprises a lipid, e.g., an ionizable lipid, disclosed in PCT Application PCT/US2022/031383, which is incorporated by reference herein, in its entirety.

In some embodiments, an LNP described herein comprises an ionizable lipid of Table 2:

TABLE 2
Exemplary Ionizable Lipids

| Compound # | Structure |
|---|---|
| L-1 | 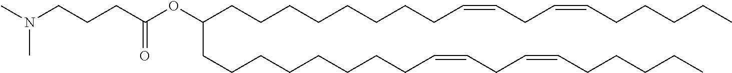 [SMILES: CCCCC/C=C\C/C=C\CCCCCCCCC(CCCCCCCC/C=C\C/C=C\CCCCC)OC(=O)CCCN(C)C] |
| L-2 | 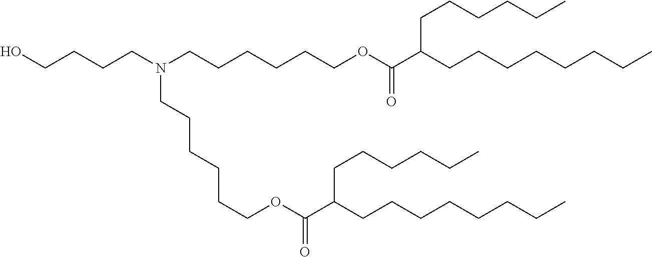 [SMILES: CCCCCCCCC(CCCCCC)C(=O)OCCCCCCN(CCCCO)CCCCCCOC(=O)C(CCCCCC)CCCCCCCC] |
| L-3 | 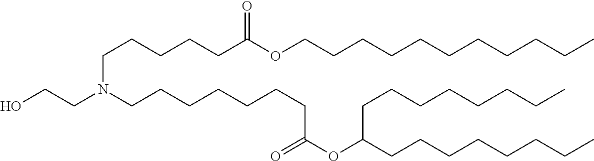 [SMILES: CCCCCCCCCCCOC(=O)CCCCCN(CCO)CCCCCCCC(=O)OC(CCCCCCCC)CCCCCCCC] |
| L-4 | 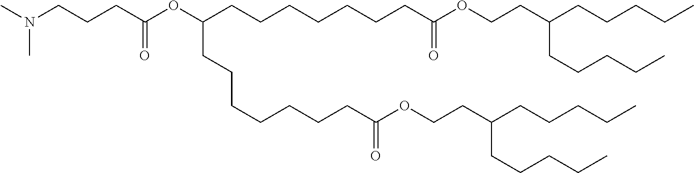 [SMILES: CCCCCC(CCCCC)CCOC(=O)CCCCCCCC(CCCCCCCC(=O)OCCC(CCCCC)CCCCC)OC(=O)CCCN(C)C] |
| L-5 | 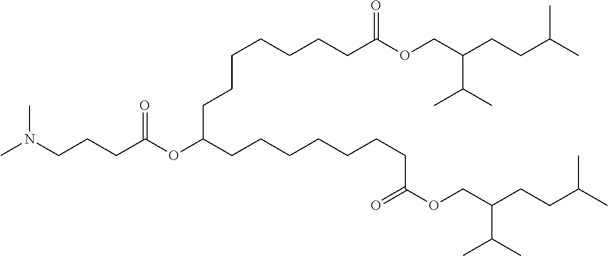 [SMILES: CC(C)CCC(COC(=O)CCCCCCCC(CCCCCCCC(=O)OCC(CCC(C)C)C(C)C)OC(=O)CCCN(C)C)C(C)C] |
| L-6 | 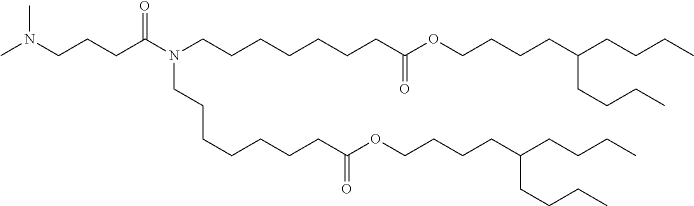 [SMILES: CCCCC(CCCC)CCCCOC(=O)CCCCCCCN(CCCCCCCC(=O)OCCCCC(CCCC)CCCC)C(=O)CCCN(C)C] |
| L-7 | 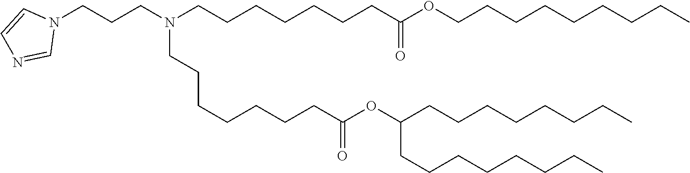 [SMILES: CCCCCCCCCOC(=O)CCCCCCCN(CCCCCCCC(=O)OC(CCCCCCCC)CCCCCCCC)CCCn1ccnc1] |
| L-8 | 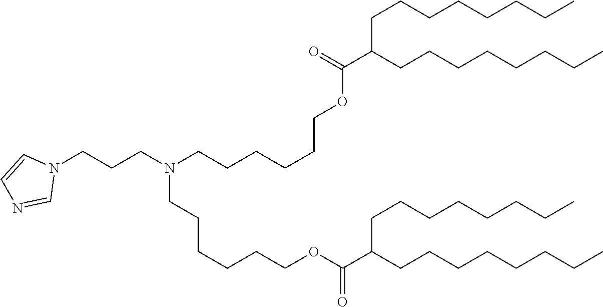 [SMILES: CCCCCCCCC(CCCCCCCC)C(=O)OCCCCCCN(CCCCCCOC(=O)C(CCCCCCCC)CCCCCCCC)CCCn1ccnc1] |
| L-9 | 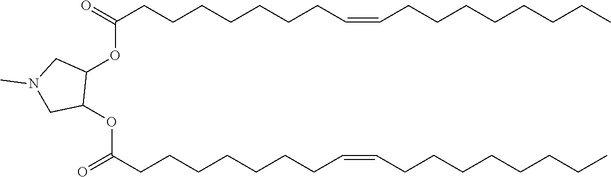 [SMILES: CCCCCCCC/C=C\CCCCCCCC(=O)OC1CN(C)CC1OC(=O)CCCCCCC/C=C\CCCCCCCC] |
| L-10 | 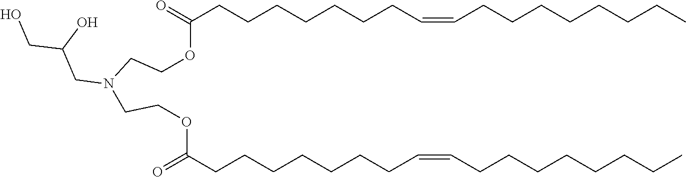 [SMILES: CCCCCCCC/C=C\CCCCCCCC(=O)OCCN(CCOC(=O)CCCCCCC/C=C\CCCCCCCC)CC(O)CO] |

b. Structural Lipids

In some embodiments, an LNP comprises a structural lipid. Structural lipids can be selected from the group consisting of, but are not limited to, cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or any combinations thereof. In some embodiments, a structural lipid is described in international patent application WO2019152557A1, which is incorporated herein by reference in its entirety.

In some embodiments, a structural lipid is a cholesterol analog. Using a cholesterol analog may enhance endosomal escape as described in Patel et al., Naturally-occuring cholesterol analogues in lipid nanoparticles induce polymorphic shape and enhance intracellular delivery of mRNA, Nature Communications (2020), which is incorporated herein by reference.

In some embodiments, a structural lipid is a phytosterol. Using a phytosterol may enhance endosomal escape as described in Herrera et al., Illuminating endosomal escape of polymorphic lipid nanoparticles that boost mRNA delivery, Biomaterials Science (2020), which is incorporated herein by reference.

In some embodiments, a structural lipid contains plant sterol mimetics for enhanced endosomal release.

c. PEGylated Lipids

A PEGylated lipid is a lipid modified with polyethylene glycol. In some embodiments, the LNP comprises a compound of Formula I or a pharmaceutically acceptable salt thereof, as described herein above. In some embodiments, the LNP comprises a compound of Formula II or a pharmaceutically acceptable salt thereof, as described herein above.

In some embodiments, an LNP comprises an additional PEGylated lipid or PEG-modified lipid. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the LNP comprises a PEGylated lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; or WO 2019/089828A1, each of which is incorporated by reference herein in their entirety. d. Phospholipids

In some embodiments, an LNP of the present disclosure comprises a phospholipid. Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In some embodiments, an LNP includes DSPC. In certain embodiments, an LNP includes DOPE. In some embodiments, an LNP includes both DSPC and DOPE.

In some embodiments, a phospholipid tail may be modified in order to promote endosomal escape as described in U.S. 2021/0121411, which is incorporated herein by reference.

In some embodiments, the LNP comprises a phospholipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, phospholipids disclosed in US 2020/0121809 have the following structure:

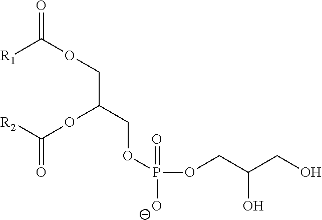

wherein R₁ and R₂ are each independently a branched or straight, saturated or unsaturated carbon chain (e.g., alkyl, alkenyl, alkynyl).

e. Targeting Moieties

In some embodiments, the lipid nanoparticle further comprises a targeting moiety. The targeting moiety may be an antibody or a fragment thereof. The targeting moiety may be capable of binding to a target antigen.

In some embodiments, the pharmaceutical composition comprises a targeting moiety that is operably connected to a lipid nanoparticle. In some embodiments, the targeting moiety is capable of binding to a target antigen. In some embodiments, the target antigen is expressed in a target organ.

In some embodiments, the target antigen is expressed more in the target organ than it is in the liver.

In some embodiments, the targeting moiety is an antibody as described in WO2016189532A1, which is incorporated herein by reference. For example, in some embodiments, the targeted particles are conjugated to a specific anti-CD38 monoclonal antibody (mAb), which allows specific delivery of the siRNAs encapsulated within the particles at a greater percentage to B-cell lymphocytes malignancies (such as MCL) than to other subtypes of leukocytes.

In some embodiments, the lipid nanoparticles may be targeted when conjugated/attached/associated with a targeting moiety such as an antibody.

f. Zwitterionic Amino Lipids

In some embodiments, an LNP comprises a zwitterionic lipid. In some embodiments, an LNP comprising a zwitterionic lipid does not comprise a phospholipid.

Zwitterionic amino lipids have been shown to be able to self-assemble into LNPs without phospholipids to load, stabilize, and release mRNAs intracellular as described in U.S. Patent Application 20210121411, which is incorporated herein by reference in its entirety. Zwitterionic, ionizable cationic and permanently cationic helper lipids enable tissue-selective mRNA delivery and CRISPR-Cas9 gene editing in spleen, liver and lungs as described in Liu et al., Membrane-destablizing ionizable phospholipids for organ-selective mRNA delivery and CRISPR-Cas gene editing, Nat Mater. (2021), which is incorporated herein by reference in its entirety.

The zwitterionic lipids may have head groups containing a cationic amine and an anionic carboxylate as described in Walsh et al., Synthesis, Characterization and Evaluation of Ionizable Lysine-Based Lipids for siRNA Delivery, Bioconjug Chem. (2013), which is incorporated herein by reference in its entirety. Ionizable lysine-based lipids containing a lysine head group linked to a long-chain dialkylamine through an amide linkage at the lysine a-amine may reduce immunogenicity as described in Walsh et al., Synthesis, Characterization and Evaluation of Ionizable Lysine-Based Lipids for siRNA Delivery, Bioconjug Chem. (2013).

g. Additional Lipid Components

In some embodiments, the LNP compositions of the present disclosure further comprise one or more additional lipid components capable of influencing the tropism of the LNP. In some embodiments, the LNP further comprises at least one lipid selected from DDAB, EPC, 14PA, 18BMP, DODAP, DOTAP, and C12-200 (see Cheng, et al. Nat Nanotechnol. 2020 April; 15(4): 313-320.; Dillard, et al. PNAS 2021 Vol. 118 No. 52.).

h. Pharmaceutical Compositions

In some embodiments, a nanoparticle includes an ionizable lipid, a phospholipid, a PEG lipid, and a structural lipid. In certain embodiments, the lipid component of the nanoparticle composition includes about 30 mol % to about 60 mol % ionizable lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the nanoparticle composition includes about 35 mol % to about 55 mol % ionizable lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % ionizable lipid, about 10 mol % phospholipid, about 38.5 mol % structural lipid, and about 1.5 mol % of PEG lipid. In another particular embodiment, the lipid component includes about 40 mol % ionizable lipid, about 20 mol % phospholipid, about 38.5 mol % structural lipid, and about 1.5 mol % of PEG lipid. In another particular embodiment, the lipid component includes about 48.5 mol % ionizable lipid, about 10 mol % phospholipid, about 40 mol % structural lipid, and about 1.5 mol % of PEG lipid. In another particular embodiment, the lipid component includes about 48.5 mol % ionizable lipid, about 10 mol % phospholipid, about 39 mol % structural lipid, and about 2.5 mol % of PEG lipid. In some embodiments, the phospholipid may be DOPE or DSPC. In other embodiments, the PEG lipid may be PEG-DMG and/or the structural lipid may be cholesterol. The amount of active agent in a nanoparticle composition may depend on the size, composition, desired target and/or application, or other properties of the nanoparticle composition as well as on the properties of the active agent. For example, the amount of active agent useful in a nanoparticle composition may depend on the size, sequence, and other characteristics of the active agent. The relative amounts of active agent and other elements (e.g., lipids) in a nanoparticle composition may also vary. In some embodiments, the wt/wt ratio of the lipid component to an enzyme in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. The amount of a enzyme in a nanoparticle composition may, for example, be measured using absorption spectroscopy (e.g., ultraviolet-visible spectroscopy).

In some embodiments, a nanoparticle composition comprising an active agent of the present disclosure is formulated to provide a specific E:P ratio. The E:P ratio of the composition refers to the molar ratio of nitrogen atoms in one or more lipids to the number of phosphate groups in an RNA active agent. In general, a lower E:P ratio is preferred. The one or more enzymes, lipids, and amounts thereof may be selected to provide an E:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the E:P ratio may be from about 2:1 to about 8:1. In other embodiments, the E:P ratio is from about 5:1 to about 8:1. For example, the E:P ratio may be about 5.0:1, about 5.5:1, about 5.67:1, about 6.0:1, about 6.5:1, or about 7.0:1.

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition. Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure Zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, Such as particle size, polydispersity index, and Zeta potential.

The mean size of a nanoparticle composition may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25.

The Zeta potential of a nanoparticle composition may be used to indicate the electrokinetic potential of the composition. For example, the Zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the Zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV, to about +20 mV, from about 0 mV to about +15 mV, from about 0 mV to about +10 mV, from about 0 mV to about +5 mV, from about +5 mV to about +20 mV, from about +5 mV, to about +15 mV, or from about +5 mV to about +10 mV.

The efficiency of encapsulation of a payload describes the amount of payload that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of payload in a solution containing the nanoparticle composition before and after breaking up the nanoparticle composition with one or more organic solvents or detergents. Fluorescence may be used to measure the amount of free payload in a solution. For the nanoparticle compositions described herein, the encapsulation efficiency of a therapeutic and/or prophylactic may be at least 50%, for example 50%, 55%, 60%. 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

Lipids and their method of preparation are disclosed in, e.g., U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548 and Semple et. al, Nature Biotechnology, 2010, 28, 172-176, the full disclosures of which are herein incorporated by reference in their entirety for all purposes.

A nanoparticle composition may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro: Lippincott, Williams & Wilkins, Baltimore, Md., 2006).

iii. Non Lipid Nanoparticle

In some embodiments, the nanoparticle is a non-lipid-based nanoparticle. Non-lipid-based nanoparticles include, but are not limited to, silicon-based nanoparticles (e.g., porous silicon nanoparticles), gold nanoparticles, polypeptide-based nanoparticles, nucleotide-based nanoparticles, and carbon-based nanoparticle.

iv. Exosomes

In some embodiments, the delivery vehicle comprises at least one exosome. As used herein, “exosomes” refer to small membrane bound vesicles with an endocytic origin. Without wishing to be bound by theory, exosomes are generally released into an extracellular environment from host/progenitor cells post fusion of multivesicular bodies the cellular plasma membrane. As such, exosomes will tend to include components of the progenitor membrane in addition to designed components and cargos. Exosome membranes are generally lamellar, composed of a bilayer of lipids, with an aqueous inter-nanoparticle space.

In some embodiments, an exosome may include at least one identifier moiety as shown in FIG. 5. Non-limiting examples of an identifier moiety include glycans, antibodies, peptides, small molecules, polynucleotides (e.g., oRNA, circular DNA) and any combination thereof.

v. Liposomes

In some embodiments, the delivery vehicles comprise of at least one liposome. As used herein, “liposomes” are small vesicles comprised of at least one lipid bilayer membrane surrounding an aqueous inner-nanoparticle space that is generally not derived from a progenitor/host cell. Liposomes can be (large) multilamellar vesicle (MLV), potentially hundreds of nanometers in diameter comprising a series of concentric bilayers separated by narrow aqueous spaces, small unicellular vesicle (SUV), potentially smaller than 50 nm in diameter, and a large unilamellar vesicle (LUV), potentially between 50 and 500 nm in diameter. In some embodiments, liposomes may be comprised of any or all the same components and same component amounts as a lipid nanoparticle, differing principally in their method of manufacture.

vi. Micelles

In some embodiments, the delivery vehicles comprise of at least one micelle. As used herein, “micelles” refer to small particles which do not have an aqueous intra-particle space. Without wishing to be bound by theory, the intra-particle space of micelles is occupied by the hydrophobic tails of the lipids comprising the micelle membrane and possible associated cargo, rather than any additional lipid-head groups. In some embodiments, micelles may be comprised of any or all the same components as a lipid-nanoparticle, differing principally in their method of manufacture.

In some embodiments, a micelle may include at least one identifier moiety as shown in FIG. 5. Non-limiting examples of an identifier moiety include glycans, antibodies, peptides, small molecules, polynucleotides (e.g., circular DNA, oRNA), and any combination thereof. vii. Viral Particle

In some embodiments, the delivery vehicle comprises at least one virus like particle. As used herein, “virus like particles” refer to a vesicle predominantly of a protein capsid, sheath, shell, or coat (all used interchangeably herein) derived from a virus which can be loaded with a cargo moiety. In general, virus like particle are non-infection and may be synthesized using cellular machinery to express viral capsid protein sequences, which then self-assemble and incorporate the associated cargo moiety, though it is possible to form virus like particles by providing the capsid and cargo components without expression related cellular machinery and allowing them to self-assemble.

In some embodiments, the virus like particle may be derived from at least one of species of virus such as, but not limited to, Parvoviridae, Retroviridae, Flaviviridae, Paramyxoviridae, and bacteriophages. In some embodiments, the virus like particle may be derived from an adeno-associated virus, HIV, Hepatitis C virus, HPV, or any combination thereof.

In some embodiments, the virus like particle is an AAV particle and the AAV particle may include at least one identifier moiety as shown in FIG. 5. Non-limiting examples of an identifier moiety include glycans, antibodies, peptides, small molecules, polynucleotides (e.g., circular DNA, oRNA) and any combination thereof.

viii. Polymeric delivery technology

In some embodiments, the delivery vehicle may comprise at least one polymeric delivery agent. As used herein, “polymeric delivery agents” refer to non-aggregating delivery agents comprised of soluble polymers conjugated to cargo moieties via various linkage groups. In some embodiments, polymeric delivery agents may comprise any of the polymers described herein.

ix. Tracking Systems

The tropism discovery platform disclosed herein may utilize a variety of tracking systems which include identifier sequences and moieties (also referred to as a “barcode” e.g., nucleotide barcode) in order to allow qualification of the delivery vehicles and/or the benchmark constructs, cargo and payloads post-administration.

In some embodiments, the tracking system is a single identifier sequence or moiety. The identifier sequence or moiety may be located in the delivery vehicle, benchmark construct, cargo or payload region, 5′ UTR, 3′UTR, promoter region or tailing region. As a non-limiting example, the identifier sequence or moiety is located in or on the delivery vehicle. As a non-limiting example, the identifier sequence or moiety is located in or on the benchmark construct. As a non-limiting example, the identifier sequence or moiety is located in or on the 5′ UTR. As a non-limiting example, the identifier sequence or moiety is located in or on the 3′ UTR. As a non-limiting example, the identifier sequence or moiety is located in or on the promoter region. As a non-limiting example, the identifier sequence or moiety is located in or on the payload region. As a non-limiting example, the identifier sequence or moiety is located in or on the tailing region.

In some embodiments, the tracking system is a set of identifier sequences or moieties with a first identifier sequence or moiety for the delivery vehicle and a second identifier sequence or moiety for the benchmark construct, cargo and payload. The first and second identifier sequence or moiety may be the same or different. If there are additional benchmark constructs, cargos and payloads in the delivery vehicle then each benchmark constructs, cargo and payloads may have its own identifier sequence or moiety or it may be the same at the second identifier sequence or moiety.

In some embodiments, the tropism discovery platform is comprised of multiple tracking systems, wherein each tracking system allows for detecting the delivery vehicle and/or benchmark constructs, cargo and payloads at different levels of resolution.

In some embodiments, the tracking systems comprises at least one barcode sequence. As used herein, a “barcode” or “barcode sequence” is any sequence which can be detected using methods known in the art and is distinct from the sequences in the cell, tissue, organ and/or organism or any sequences being administered. The barcode sequence may be included in or attached to the delivery vehicle and/or in the benchmark construct, cargo and payload. As a non-limiting example, the delivery vehicle comprises the barcode sequence. As a non-limiting example, the cargo or payload comprises the barcode sequence. As a non-limiting example, the benchmark construct comprises the barcode sequence.

In some embodiments, the location of the identifier sequence or moiety in the targeting system is random. As a non-limiting example, the identifier sequence or moiety is in the delivery vehicle. As a non-limiting example, the identifier sequence or moiety is in the delivery vehicle. As a non-limiting example, the identifier sequence or moiety is in the benchmark construct. As a non-limiting example, the identifier sequence or moiety is in the cargo or payload. As a non-limiting example, the identifier sequence or moiety is in the delivery vehicle and the benchmark construct. As a non-limiting example, the identifier sequence or moiety is in the delivery vehicle and the cargo or payload. As a non-limiting example, the identifier sequence or moiety is in the benchmark construct and the cargo or payload. As a non-limiting example, the identifier sequence or moiety is in the delivery vehicle, benchmark construct, and the cargo or payload.

In some embodiments, the location of the identifier sequence or moiety in the targeting system is pre-determined. As a non-limiting example, the identifier sequence or moiety is in the delivery vehicle. As a non-limiting example, the identifier sequence or moiety is in the delivery vehicle. As a non-limiting example, the identifier sequence or moiety is in the benchmark construct. As a non-limiting example, the identifier sequence or moiety is in the cargo or payload. As a non-limiting example, the identifier sequence or moiety is in the delivery vehicle and the benchmark construct. As a non-limiting example, the identifier sequence or moiety is in the delivery vehicle and the cargo or payload. As a non-limiting example, the identifier sequence or moiety is in the benchmark construct and the cargo or payload. As a non-limiting example, the identifier sequence or moiety is in the delivery vehicle, benchmark construct, and the cargo or payload.

In some embodiments, the location of the identifier sequence or moiety in the targeting system is inverted. As a non-limiting example, the identifier sequence or moiety is in the delivery vehicle. As a non-limiting example, the identifier sequence or moiety is in the delivery vehicle. As a non-limiting example, the identifier sequence or moiety is in the benchmark construct. As a non-limiting example, the identifier sequence or moiety is in the cargo or payload. As a non-limiting example, the identifier sequence or moiety is in the delivery vehicle and the benchmark construct. As a non-limiting example, the identifier sequence or moiety is in the delivery vehicle and the cargo or payload. As a non-limiting example, the identifier sequence or moiety is in the benchmark construct and the cargo or payload. As a non-limiting example, the identifier sequence or moiety is in the delivery vehicle, benchmark construct, and the cargo or payload.

In some embodiments, the identifier sequence is a randomly generated sequences which serve to avoid duplication during deep sequencing. In some embodiments, the identifier sequence is a repeating sequence of nucleotides or amino acids. In some embodiments, the identifier sequence is a fragment of a larger sequence such as, but not limited to, a cargo or payload. The identifier sequence may be designed to any length available using synthesis technology (See Clement et al, AmpUMI. design and analysis of unique molecular identifiers for deep amplicon sequencing, Bioinformatics, Volume 34, Issue 13, 1 Jul. 2018, Pages i202-i210; the contents of which is herein incorporated herein by reference in its entirety).

In some embodiments, the identifier sequence has a length between 2 and 1000 nucleotides. For example, the identifier sequence may have a length of 2, 3, 4, 5, 6, 7, 8, 9, 10, 20, 21, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 or more than 1000 nucleotides. The identifier sequence may have a length between 2-5, 2-10, 2-15, 2-20, 2-30, 2-50, 2-70, 2-90, 2-100, 2-250, 2-300, 2-350, 2-400, 2-450, 2-500, 2-550, 2-600, 2-650, 2-700, 2-750, 2-800, 2-850, 2-900, 2-950, 2-1000, 5-10, 5-15, 5-20, 5-30, 5-50, 5-70, 5-90, 5-100, 5-250, 5-300, 5-350, 5-400, 5-450, 5-500, 5-550, 5-600, 5-650, 5-700, 5-750, 5-800, 5-850, 5-900, 5-950, 5-1000, 10-30, 10-50, 10-70, 10-90, 10-100, 10-250, 10-300, 10-350, 10-400, 10-450, 10-500, 10-550, 10-600, 10-650, 10-700, 10-750, 10-800, 10-850, 10-900, 10-950, 10-1000, 20-30, 20-50, 20-70, 20-90, 20-100, 20-250, 20-300, 20-350, 20-400, 20-450, 20-500, 20-550, 20-600, 20-650, 20-700, 20-750, 20-800, 20-850, 20-900, 20-950, 20-1000, 30-50, 30-70, 30-90, 30-100, 30-250, 30-300, 30-350, 30-400, 30-450, 30-500, 30-550, 30-600, 30-650, 30-700, 30-750, 30-800, 30-850, 30-900, 30-950, 30-1000, 40-50, 40-70, 40-90, 40-100, 40-250, 40-300, 40-350, 40-400, 40-450, 40-500, 40-550, 40-600, 40-650, 40-700, 40-750, 40-800, 40-850, 40-900, 40-950, 40-1000, 50-70, 50-90, 50-100, 50-250, 50-300, 50-350, 50-400, 50-450, 50-500, 50-550, 50-600, 50-650, 50-700, 50-750, 50-800, 50-850, 50-900, 50-950, 50-1000, 60-70, 60-90, 60-100, 60-250, 60-300, 60-350, 60-400, 60-450, 60-500, 60-550, 60-600, 60-650, 60-700, 60-750, 60-800, 60-850, 60-900, 60-950, 60-1000, 70-90, 70-100, 70-250, 70-300, 70-350, 70-400, 70-450, 70-500, 70-550, 70-600, 70-650, 70-700, 70-750, 70-800, 70-850, 70-900, 70-950, 70-1000, 80-90, 80-100, 80-250, 80-300, 80-350, 80-400, 80-450, 80-500, 80-550, 80-600, 80-650, 80-700, 80-750, 80-800, 80-850, 80-900, 80-950, 80-1000, 90-100, 90-250, 90-300, 90-350, 90-400, 90-450, 90-500, 90-550, 90-600, 90-650, 90-700, 90-750, 90-800, 90-850, 90-900, 90-950, 90-1000, 100-250, 100-300, 100-350, 100-400, 100-450, 100-500, 100-550, 100-600, 100-650, 100-700, 100-750, 100-800, 100-850, 100-900, 100-950, 100-1000, 150-250, 150-300, 150-350, 150-400, 150-450, 150-500, 150-550, 150-600, 150-650, 150-700, 150-750, 150-800, 150-850, 150-900, 150-950, 150-1000, 200-250, 200-300, 200-350, 200-400, 200-450, 200-500, 200-550, 200-600, 200-650, 200-700, 200-750, 200-800, 200-850, 200-900, 200-950, 200-1000, 250-300, 250-350, 250-400, 250-450, 250-500, 250-550, 250-600, 250-650, 250-700, 250-750, 250-800, 250-850, 250-900, 250-950, 250-1000, 300-350, 300-400, 300-450, 300-500, 300-550, 300-600, 300-650, 300-700, 300-750, 300-800, 300-850, 300-900, 300-950, 300-1000, 350-400, 350-450, 350-500, 350-550, 350-600, 350-650, 350-700, 350-750, 350-800, 350-850, 350-900, 350-950, 350-1000, 400-450, 400-500, 400-550, 400-600, 400-650, 400-700, 400-750, 400-800, 400-850, 400-900, 400-950, 400-1000, 450-500, 450-550, 450-600, 450-650, 450-700, 450-750, 450-800, 450-850, 450-900, 450-950, 450-1000, 500-550, 500-600, 500-650, 500-700, 500-750, 500-800, 500-850, 500-900, 500-950, 500-1000, 550-600, 550-650, 550-700, 550-750, 550-800, 550-850, 550-900, 550-950, 550-1000, 600-650, 600-700, 600-750, 600-800, 600-850, 600-900, 600-950, 600-1000, 650-700, 650-750, 650-800, 650-850, 650-900, 650-950, 650-1000, 700-750, 700-800, 700-850, 700-900, 700-950, 700-1000, 750-800, 750-850, 750-900, 750-950, 750-1000, 800-850, 800-900, 800-950, 800-1000, 850-900, 850-950, 850-1000, 900-950, 900-1000, 950-1000 or over 1000 nucleotides.

In some embodiments, the identifier sequence or moiety may produce a signal that is detectable immediately after administration. In some embodiments, the identifier sequence or moiety may produce a signal that is detectable for an indefinite amount of time after administration. In some embodiments, the identifier sequence or moiety may produce a signal that is detectable for more than 1 day, 2 days, 3 days, 4 days, 5 days, 6 days or 7 days post administration. In some embodiments, the identifier sequence or moiety may produce a signal that is detectable for about 1 to 24 hours. As a non-limiting example, the signal may be detectable for about 1 to 6, 1 to 12, 1 to 18, 6 to 12, 6 to 18,6 to 24, or 18 to 24 hours, or 1,2,3,4, 5,6,7, 8,9, 10, 11, 12, 13, 14, 15, 16, 17, 18, 19,20,21, 22, 23, or 24 hours. In some embodiments, the identifier sequence or moiety may produce a signal that is detectable for about 1-60 minutes such as, but not limited to, 1-5, 1-10, 1-20, 1-30, 1-40, 1-50, 10-20, 10-30, 10-40, 10-50, 10-60, 20-30, 20-40, 20-50, 20-60, 30-40, 30-50, 30-60, 40-50, 40-60, or 50-60 minutes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes. In some embodiments, the identifier sequence or moiety may produce a signal that is detectable for less than 1 minute post administration.

In some embodiments, the identifier sequence or moiety may produce a signal that is detectable from outside the body of a subject. In some embodiments, the identifier sequence or moiety may produce a signal that is detectable from via non-invasive imagery techniques, for example from outside a subject's organs or tissues but within the subject's body. In some embodiments, the identifier sequence or moiety may produce a signal that is detectable on a macroscopic level. In some embodiments, the identifier sequence or moiety may produce a signal that is detectable on the microscopic level. In some embodiments, the identifier sequence or moiety may produce a signal that is detectable on the nanoscopic level. In some embodiments, the identifier sequence or moiety may produce a signal that is only detectable after target cells are harvested and assayed, for non-limiting example via mass spectrometer, electrophoresis, flow cytometry, or deep sequencing.

x. Nucleotide Barcode Identifier Sequences

Detection of the unique identifier to produce a qualitative and/or quantitative determination of the tissue/organ/cell containing a targeting system, and identification of each specific targeting system (e.g., delivery system, payload), can be carried out with any suitable method, of which there are many known in the art, including, but not limited to MRI, sequencing, radio imaging, fluorescence microscopy, DNA sequencing, flow cytometry and the like. One suitable method for detection is nucleotide sequencing of a unique identifier sequence (e.g., a linear DNA barcode, a linear RNA barcode, a circular DNA barcode, a circular RNA barcode), for example to identify the targeting systems (e.g., delivery vehicle, payload, promoter) present and optionally quantify a targeting system relative to other targeting systems and/or relative to endogenous polynucleotides of the target (e.g., cell, tissue, organ, subject).

A nucleotide barcode identifier sequence is a nucleotide sequence that can be of any length. Nucleotide barcode identifier sequences of this disclosure typically range from 6 to about 21 nucleotides. In some embodiments, preferable nucleotide barcode identifier sequences of the present disclosure are 6, 7, 8, 9, 10, 11, or 12 nucleotides in length. Nucleotide barcode identifier sequences can be used to encode information that can be used, for example, to identify particular members of a collection of a library of molecules. For example, a nucleotide barcode identifier sequence of 10 nucleotides comprised of the standard four ribonucleosides or the standard four deoxyribonucleosides (e.g., adenosine, uridine, cytidine, guanosine, 2′-deoxyadenosine, thymidine, 2′-deoxycytidine, and 2′-deoxyguanosine, respectively) can create a library of 1,048,576 unique sequences. This data storage capacity can be utilized to encode for information relating to many different parameters of each member in a library. This encoding can be rational, for example, through design of specific barcoding sequences for each library member. For example, each library member in a targeting system library can be cataloged by (e.g., associated with or linked to) a unique barcode sequence that identifies the originator polynucleotide construct, regulatory sequences (e.g., promotor, enhancer), payloads (e.g., siRNA, oRNA, circular DNA), the delivery vehicle type (e.g., lentivirus, adenovirus, lipid nanoparticle), properties of the delivery vehicle (e.g., size, composition, production method), or targeting moieties of the delivery vehicle (e.g., glycans, peptides, antibodies, small molecules). Accordingly, this disclosure relates to targeting systems that can comprise nucleotide barcode identifier sequences that are rationally assigned to each targeting system in a library. In one alternative, this disclosure relates to targeting systems that can comprise nucleotide barcode identifier sequences that are randomized or assigned randomly to targeting systems in a library.

In some embodiments an originator polynucleotide construct of the present disclosure is an engineered polynucleotide comprising a unique nucleotide barcode identifier sequence, optionally further comprising one or more elements selected from a payload sequence region, at least one flanking sequence region and at least one regulatory sequence region. In some embodiments, the payload sequence region comprises a coding nucleic acid sequence. In some embodiments, the payload region comprises a non-coding nucleic acid sequence. In some embodiments, the originator polynucleotide construct does not comprise a payload sequence. In such instances, the originator polynucleotide construct can be co-formulated with a second engineered polynucleotide that does comprise a payload sequence.

This disclosure relates to nucleotide barcode identifier sequences comprising any suitable nucleoside building block linked by suitable linkages, such as mono-, di-, or triphosphate linkages or phosphorothioate linkages, of which there are many known in the art. Suitable nucleosides for use in barcodes include, but are not limited to, the following: 2′-deoxyribonucleosides (e.g., 2′-deoxyadenosine, 2′-deoxyguanosine, thymidine, 2′-deoxycytidine, 2′-deoxyuridine, 2′-deoxyinosine,), ribonucleosides (e.g., adenosine, guanosine, uridine, cytidine, inosine), methylated nucleosides (e.g., 5-methyluridine, 2′-deoxy-5-methylcytidine, 2′-deoxy-N⁶-methyladenosine, N⁶-methyladenosine, 2′-deoxy-N⁴-methylcytidine, N⁴-methylcytidine, N¹-methyladenosine, 2′-deoxy-N¹-methyladenosine, N⁶, 2′-O-dimethyladenosine, N⁷-methylguanosine, 2′-deoxy-N⁷-methylguanosine, 5-hydroxymethylcytidine, 2′-deoxy-5-hydroxymethylcytidine, N^{N}-methylcytidine, 2′-deoxy-N³-methylcytidine, 2′-O-methyladenosine, 2′-O-methyluridine, 2′-O-methyl-cytidine, 2′—O-methylguanosine), acetylated nucleosides (e.g., N⁴-acetylcytidine, 2′-deoxy-N⁴-acetylcytidine), other modified nucleosides (e.g., pseudouridine (Ψ), N¹-methyl-pseudouridine (N¹-methyl-Ψ), 2-thiouridine (s2U), 5-fluoro-2′-deoxyuridine (FUDR), 8-oxo-7,8-dihydroguanosine (8-oxoG), 2′-deoxy-2′-fluoro-ribonucleosides, 2′-O-methoxyribonucleosides, N-ethylpiperidine-7-EAA triazole modified adenine, N-ethylpiperidine-6-triazole modified adenosine, 6-phenylpyrrolo-cytosine (PhpC), 2,4-difluorotoluyl-ribonucleoside (rF), N¹(5-nitroindole) ribonucleoside, 2′-O-methyl-ribonucleosides (2′-OMe), 2′-O-methoxyethyl-ribonucleosides (2′-O-MOE), 2′-deoxy-2′-fluororibonucleosides (2′-F), 2′-deoxyarbino-2′-fluoronucleosides (2′-Ara-F), 2′-O-benzyl-2′-deoxyribonucleosides, 2′-O-methyl-4-pyridinylribonucleosides (2′-O-CH2Py(4)), 5-methoxyuridine) hybrids thereof, mixtures thereof, or combinations thereof. Suitable linkages for nucleotides comprised of nucleosides include but are not limited to phosphodiester linkages, phosphorothioate linkages, methylphosphonate linkages, phosphoramidate linkages, and phosphorodiamidate linkages. For example, a nucleotide barcode identifier sequence of this disclosure can comprise a circular RNA comprising phosphorothioate linkages between nucleosides and pseudouridine nucleosides. As another example, a nucleotide barcode identifier sequence of this disclosure can comprise a circular DNA comprising 2′-deoxy-5-methylcytidine, 2′-deoxy-N⁶-methyladenosine nucleosides.

This disclosure relates to nucleotide barcode identifier sequences comprising any nucleic acid including, but not limited to ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, a-LNA having an a-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA), or hybrids thereof, or combinations thereof. For example, a nucleotide barcode identifier sequence of this disclosure can comprise peptide nucleic acids wherein such a barcode is identified, detected and quantified by liquid chromatography-mass spectrometry.

This disclosure relates to nucleotide barcode identifier sequences comprising or as part of any suitable polynucleotide structure. Suitable polynucleotide structures include, but are not limited to linear double-stranded structures (e.g., double-stranded linear DNA, double-stranded RNA), linear single-stranded structures (e.g., mRNA, DNA), circular double-stranded structures (e.g., plasmid DNA, covalently closed circular DNA (cccDNA), double-stranded oRNA, double-stranded circular DNA), single-stranded bulged structures, single-stranded loop structures (e.g., hairpin RNA, hairpin DNA), single-stranded helical structures, single-stranded junction structures (e.g., RNA multibranch loop), double-stranded pseudoknot structures, tetra-stranded junction (e.g., Holliday junction), single-stranded circular structures (e.g., circular RNA, circular single-stranded DNA), hybrid structures thereof, and combinations thereof.

In preferred aspects, this disclosure relates to a library of targeting systems (e.g., suitable for detecting tropism) comprising nucleotide barcode identifier sequences wherein the nucleotide barcode identifier sequence is part of a circular RNA. Without intending to be limited to any particular theory, circular RNA comprising one or more unique barcode identifier sequences are especially attractive for use in the methods of the present disclosure because circular RNA has been shown to have improved expression, functional stability in vivo, lower immunogenicity, increased ease of manufacturing and/or extended half-life compared to analogous linear RNAs (see, for example, Front. Mol. Biosci., 10 Jan. 2020 Sec. RNA Networks and Biology). Additionally, it has surprisingly been found that pharmaceutical delivery vehicles can be formulated to contain more circular RNA than analogous linear RNA. Without intending to be limited to any particular theory, this observation is likely due to tighter packing efficiency in the delivery vehicle by the more structured circular RNA, as compared to the linear RNA. In some embodiments, this greater concentration of circular RNA comprising unique barcode identifier sequences can yield greater resolution in methods of the present disclosure for determining the biodistribution of the barcoded circular RNAs and their associated compounds.

Nucleotide barcode identifier sequences of this disclosure typically comprise from about 1 to about 21 nucleotides, e.g., about 1 to about 3, about 3 to about 6, about 6 to about 9, about 9 to about 12, about 12 to about 15, about 15 to about 18, about 18 to about 21, about 2 to about 10 or about 10 to about 21 nucleotides. In some embodiments, nucleotide barcode identifier sequences comprise about 5 to about 10 nucleotides. In some embodiments, nucleotide barcode identifier sequences comprise about 5 to 20 nucleotides. This disclosure relates to targeting systems that can comprise a nucleotide barcode identifier sequence of 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, or 21 nucleotides. In some embodiments, the nucleotide barcode identifier sequences comprise more than 21 nucleotides, as described elsewhere herein.

The nucleotide barcode identifier sequence associated with a targeting system of this disclosure can be associated by or linked through any desired approach, such as, by a covalent bond or a non-covalent interaction (e.g., electrostatic, hydrophobic). This disclosure relates to targeting systems comprising a nucleotide barcode identifier sequence attached through a covalent bond or non-covalent interaction to any element (e.g. polynucleotide construct, promoter, payload, cargo, delivery vehicle) of the targeting system. The nucleotide barcode identifier sequence of targeting systems of this disclosure can be attached to any region of the originator polynucleotide construct including, but not limited to a payload, cargo, a regulatory region (e.g., a promoter, an enhancer), a flanking region, and an untranslated region. The nucleotide barcode identifier sequence of targeting systems of this disclosure can be attached to any position of an originator polynucleotide construct region including, but not limited to, the 5′-end, the 5′-terminus, the 3′-end, the 3′-terminus, and between the 5′ and 3′ ends.

This disclosure also relates to targeting systems comprising a nucleotide barcode identifier sequence attached to the delivery vehicle of the targeting system. The nucleotide barcode identifier sequence can be attached by covalent bond to the delivery vehicle. The nucleotide barcode identifier sequence can be attached by non-covalent interaction to the delivery vehicle. Nucleotide barcode identifier sequences of this disclosure can be attached to delivery vehicles through many chemistries well-known to those skilled in the art including, but not limited to, biotin-streptavidin interaction, condensation reaction (e.g., oxime formation, hydrazone formation), ligation, amide bond formation, esterification, click reaction (e.g., Huisgen alkyne-azide cycloaddition, a tetrazine ligation, thiol-ene reaction, 1,3-dipolar cycloaddition), alkylation, nucleophilic epoxide opening, disulfide formation, and the like. As a non-limiting example, a nucleotide barcode identifier sequence can be synthesized with a 3′-terminus azido chemical group via solid-phase nucleotide synthesis with an appropriate azido-nucleotide building block, followed by click reaction with a lipid nanoparticle comprised of an alkynyl cationic lipid component. For example, similar methods for lipid nanoparticle modification are described in G. Yi, et. al Biomaterials Research (2018), 22, 13.

Nucleotide barcode identifier sequences of this disclosure can be included in a polynucleotide at a desired site (e.g., at a defined number of nucleotides downstream or upstream of a payload sequence, at the 5′ terminus or 3′ terminus) or a desired region (e.g., 3′ end of a payload sequence). Many methods for specific attachment or insertion of a nucleotide sequence (e.g., a nucleotide barcode, an RNA barcode, a DNA barcode) to another polynucleotide (e.g., a DNA plasmid, originator polynucleotide construct) are well-known in the art. As a non-limiting example, a specific region on the originator polynucleotide construct can be cleaved by a restriction enzyme that creates terminal overhanging sequences upon cleavage, incubated with a DNA barcode sequence containing complementary flanking regions for the resulting polynucleotide overhanging sequences, and ligated by a ligase to yield an originator polynucleotide attached to a specific site of the targeting system. As another example, a polynucleotide (e.g., originator polynucleotide construct) comprising a nucleotide barcode identifier sequence attached by a phosphodiester linkage at the 5′-end of a promoter sequence can be synthesized by solid-phase nucleotide synthesis. As another non-limiting example, a polynucleotide (e.g., originator construct) can be synthesized by solid-phase nucleotide synthesis to incorporate, either at a pre-selected position or randomly in the polynucleotide, a click chemistry label (e.g., azide, thiol, tetrazine) at one nucleotide and further reacted with a nucleotide barcode identifier sequence that is synthesized with a complementary click chemistry label (e.g., alkyne, alkene, isocyanide) to form a covalent linkage. The attachment or insertion of a nucleotide barcode identifier sequence can invert the sequence of the nucleotide barcode. As another example, the nucleotide can be attached as an inverted sequence by a phosphodiester bond to the 5′-end of a complement strand of a promoter region of an originator construct of this disclosure.

The association between the nucleotide barcode identifier sequence and another component (e.g., delivery vehicle, polynucleotide, regulatory region, payload, cargo, untranslated region) of a targeting system of this disclosure can be by any suitable bond or non-covalent intermolecular force. The nucleotide barcode identifier sequence can be attached through a suitable covalent bond. Suitable covalent bond linkages between a nucleotide barcode identifier sequence and another element of targeting systems of this disclosure include, but are not limited to, an ester linkage, an amide linkage, a disulfide linkage, a heteroalkyl linkage, carbon-carbon linkage, a thioester linkage, a phosphoramidate linkage, a phosphonate linkage, a phosphorothioate linkage, glycosidic linkage, a 1,4-disubstuted triazole linkage, a thioether linkage, a carbamide linkage, a carbamate linkage, a disubstituted aryl linkage, an oxime linkage, an acylhydrazide linkage, an acylhydrazone linkage, and a phosphodiester linkage. For example, the nucleotide barcode identifier sequence can comprise a sequence covalently attached to the originator construct polynucleotide through a phosphodiester linkage. The nucleotide barcode identifier sequence of this disclosure can be attached through a non-covalent interaction, e.g., methotrexate-dihydrofolate reductase binding, biotin-streptavidin binding, biotin-avidin binding, nucleotide base pairing. In some embodiments, the nucleotide barcode is associated with additional components of a targeting system of this disclosure through hydrophobic interactions. For example, the nucleotide barcode of the present disclosure can be associated with a lipid nanoparticle via encapsulation, such that no covalent bond is formed between the barcoded component and the components of the lipid nanoparticle. For example, the nucleotide barcode identifier sequence can be covalently attached to biotin which forms a non-covalent interaction with a streptavidin-bound polynucleotide originator construct. Suitable covalent bonds can be non-cleavable. For example, suitable covalent bonds for linking a nucleotide barcode identifier sequence to another component of a targeting system can include, but are not limited to, carbon-carbon bonds (e.g., carbon-carbon double bond, carbon-carbon single bond), nitrogen-nitrogen bonds (e.g., hydrazine), and carbon-nitrogen bonds (e.g., amide bond, substituted amine bond). Nucleotide barcode identifier sequences of this disclosure can be attached through a cleavable covalent linkage, of which there are many known in the art. This disclosure relates to cleavable covalent linkages between a nucleotide barcode identifier sequence and another element of the targeting system that are cleavable by enzymatic, chemical, or irradiative processes (e.g., oxidative cleavage in a cell, acid-catalyzed cleavage in a phagolysosome, proteolytic cleavage in a cell/tissue/organ/organism, reductive cleavage in a cell/tissue/organ/organism, chemical reagent-catalyzed oxidative cleavage, hydrogenolysis, UV-irradiation). Nucleotide barcode identifier sequences can be covalently attached to any element of the targeting element via a suitable linker, such as a cleavable linker. Suitable cleavable linkers are shown can include, but are not limited to, disulfide linkers, acid labile linkers (e.g., including dialkoxybenzyl linkers, Sieber linkers, indole linkers, t-butyl Sieber linkers), linkers cleavable by nucleophilic substitution, linkers cleavable by electrophilic substitution, photocleavable linkers (e.g., —O-nitrobenzyl linkers and nitroveratryl linkers), cleavage under reductive conditions (e.g., disulfide, benzyl, benzyloxycarbonyl-containing linkers), oxidative conditions, cleavage via use of safety-catch linkers, enzymatically cleavable peptide linkers (e.g., cathepsin L substrate peptides), and cleavage by elimination mechanisms (e.g., cyanoethyl linkers, allylic linkers). For example, the nucleotide barcode identifier sequence can be attached to the targeting system by a disulfide linkage which can be released from the targeting system by treatment with a solution containing sodium bisulfite during nucleotide isolation and purification. In another example, the nucleotide barcode identifier sequence can exist as a non-attached element of the targeting system (e.g., an unbound polynucleotide within the delivery vehicle).

This disclosure relates to targeting systems (e.g., lipid nanoparticles and collections thereof) comprising one or more nucleotide barcode identifier sequences. The targeting systems of this disclosure can comprise 1, 2, 3, 4, 5, 6, 7, 8, 9, 10, or more nucleotide barcode identifier sequences, e.g., 1 to 2, 1 to 3, 1 to 4, 1 to 5, 1 to 6, 1 to 7, 1 to 8, 1 to 9, 1 to 10 nucleotide barcode identifier sequences. In targeting systems of this disclosure comprising more than one nucleotide barcode, the nucleotide barcode identifier sequences can be the same (e.g., identical nucleotide sequence). In some examples, wherein the targeting systems of this disclosure comprises more than one nucleotide barcode, the nucleotide barcode identifier sequences can be different (e.g. non-identical nucleotide sequences). The nucleotide barcode identifier sequences can comprise any mixture of identical or non-identical sequences. For example, a targeting system of this disclosure can comprise two identical nucleotide barcode identifier sequences and one different nucleotide barcode identifier sequence. Targeting systems of this disclosure can comprise multiple barcodes attached to the same region or component of the targeting system. Targeting systems of this disclosure can comprise multiple barcodes attached to different regions of the targeting system (e.g., benchmark construct, regulatory region, flanking region, cargo, payload, delivery vehicle). For example, the targeting system can contain two nucleotide barcode identifier sequences attached to the 3′-flanking region of the payload, two nucleotide barcode identifier sequences attached to a regulatory region, and six nucleotide barcode identifier sequences attached to various components of the delivery vehicle. The attachment of each nucleotide barcode identifier sequence to each targeting system component (e.g., a payload, cargo, a regulatory region, a delivery vehicle) of targeting systems of this disclosure can be by different chemistries. For example, a targeting system can comprise two nucleotide barcode identifier sequences attached through phosphodiester linkages to an originator polynucleotide construct and one nucleotide barcode identifier sequence attached to a lipid nanoparticle delivery vehicle by a covalent click reaction (e.g., cycloaddition forming a 1,4-disubstituted triazole).

Nucleotide barcode identifier sequences of the present disclosure can be released from the target cell/tissue/organ by any appropriate method for releasing DNA or RNA, many of which are known in the art, for example: cell lysis by detergent, enzymatic, chemical and/or physical disruption, bulk cell or tissue lysis, chloroform-phenol extraction, mechanical lysis (e.g., bead beating), sonication, repeat pipetting, freeze-thaw cell rupture, enzymatic cell lysis (e.g. proteinase K), pressure lysis (e.g., autoclave), or chemical lysis (e.g., treatment with TRIZOL™). For example, cells can be lysed by vortexing in a solution containing TRIZOL™ to release and isolate DNA (e.g., circular DNA) and RNA (e.g., oRNA) from the cells.

Nucleotide barcode identifier sequences of the present disclosure can be extracted from the cellular milieu by any appropriate method for isolating DNA or RNA, many of which are known in the art, for example: chloroform-phenol extraction, ethanol precipitation, silica gel spin columns, de-salting, affinity beads (e.g., POLYA SPIN™ magnetic beads) or anion exchange. For example, a nucleotide barcode comprising an mRNA wherein the mRNA sequence comprises a polyadenylation region can be extracted from cell lysate by poly-thymidine magnetic beads.

Nucleotide barcode identifier sequences of the present disclosure can be amplified by any appropriate method for polymerizing and amplifying DNA or RNA, many of which are known in the art, for example: reverse transcriptase reaction to form cDNA (from an RNA barcode), polymerase chain reaction (PCR) (e.g., quantitative PCR [qPCR], nested PCR, hot start PCR, methylation specific PCR, reverse transcriptase PCR, multiplex PCR, polymerase cycling assembly, helicase-dependent amplification PCR, inverse PCR, intersequence-specific PCR, touchdown PCR, solid phase PCR, miniprimer PCR, ligation-mediated PCR, asymmetric PCR, digital PCR, universal fast walking), loop-mediated isothermal amplification (LAMP), nucleic acid sequence based amplification (NASBA), self-sustained sequence replication (3SR), ligase chain reaction (LCR), strand displacement amplification (SDA) and rolling circle amplification (e.g., for circular RNA). Amplification of nucleotide barcode identifier sequences of the present disclosure can be general to all nucleotide barcode identifier sequences of every targeting system in a target system library. For example, PCR can be used to amplify a nucleotide barcode identifier sequence with PCR primers complementary to 5′- and 3′-flanking regions of every nucleotide barcode identifier sequence present in a biological sample. In an aspect of this disclosure, amplification of nucleotide barcode identifier sequences can be specific to each individual nucleotide barcode identifier sequence in a targeting system library of the present disclosure. For example, a nucleotide barcode identifier sequence of this disclosure can contain unique flanking (e.g., 5′-flanking and 3′-flanking regions) regions complementary to specific PCR primers designed to amplify only one nucleotide barcode identifier sequence unique to only one targeting system in a targeting system library.

Nucleotide barcode identifier sequences of the present disclosure can be detected and quantified by any appropriate method for detecting and quantifying DNA or RNA, many of which are well-known in the art, for example: DNA gel electrophoresis, UV-Vis spectrophotometric analysis, qPCR, CRISPR-typing PCR (ctPCR), fluorometric analysis, Southern blotting, radio-probe hybridization and detection, Northern blotting, nucleic acid hybridization, and phage plaque assay. For example, a targeting system of this disclosure can comprise a circular RNA nucleotide barcode identifier sequence wherein the oRNA can be detected and quantified by reverse-transcriptase qPCR (RT-qPCR). As another example, a targeting system of this disclosure can comprise a circular DNA nucleotide barcode identifier sequence wherein the circular DNA can be detected and quantified by qPCR (qPCR). Quantification of nucleotide barcode identifier sequences of this disclosure can be relative (e.g., relative to host or subject RNA, relative to other barcodes in the targeting system library, relative to host or subject DNA, relative to nucleotides from a separate organ or tissue) or absolute (e.g., quantified in comparison to a spike-in sequence of known quantity).

Nucleotide barcode identifier sequences of the present disclosure can be sequenced and identified by any appropriate method for sequencing DNA or RNA, many of which are known in the art, for example: Sanger sequencing, capillary electrophoresis fragment analysis, next-generation sequencing (e.g., circular RNA sequencing by next-generation sequencing, shotgun metagenomic sequencing, shotgun transcriptomic sequencing, single-cell sequencing, pyrosequencing, high-throughput sequencing, and full-length circular RNA sequencing (e.g., circFL-seq). In some aspects of this disclosure, the nucleotide barcode identifier sequence is RNA and a reverse transcriptase is used to provide cDNA from the RNA, and thereafter the cDNA is sequenced to infer the RNA barcode sequence. As another example, circular RNA sequences can be sequenced and identified by many methods well-known in the art, as summarized in S. Jiao, et al. Front. Genet. (2021).

The present disclosure contemplates the use of many unique nucleotide barcode identifier sequences simultaneously, wherein each unique nucleotide barcode identifier sequence correlates to a different compound being administered to a subject. In this way, the present disclosure enables a method of determining the biodistribution of one or more different compounds in a subject after administration of the uniquely barcoded compounds.

In some embodiments, the present disclosure contemplates administering to a subject at least 2 compounds, each associated with a different unique nucleotide barcode identifier sequence. In some embodiments, the present disclosure contemplates administering to a subject at least 3 compounds, each associated with a different unique nucleotide barcode identifier sequence. In some embodiments, the present disclosure contemplates administering to a subject at least 5 compounds, each associated with a different unique nucleotide barcode identifier sequence. In some embodiments, the present disclosure contemplates administering to a subject at least 10 compounds, each associated with a different unique nucleotide barcode identifier sequence. In some embodiments, the present disclosure contemplates administering to a subject at least 12, 15, 20, 25, 30, 35, 40, 45, 50, 55, 60, 65, 70, 75, 80, 85, 90, 95, or 100 compounds, each associated with a different unique nucleotide barcode identifier sequence.

xi. Tracking System: Fluorescence

In some embodiments, the at least one tracking system comprises an identifier sequence or moieties that is detectable by florescence.

In some embodiments, florescence is achieved via the inclusion of at least one fluorescent dye in the delivery vehicle. In some embodiments, the at least one fluorescent dye may be selected from, but is not limited to, fluorescein, TAMRA (carboxytetramethylrhodamine), Cy dyes, Texas red, HEX, JOE, Oregon green, rhodamine 6 G, coumarin, pyrene, and DiOC6 (3,3′-dihexyloxacarbocyanine iodide).

In some embodiments, florescence is achieved via the inclusion of at least one fluorescent protein in the or associated with the delivery vehicle. In some embodiments, at least one fluorescent protein is encoded in the benchmark construct or the benchmark construct comprises the fluorescent protein. Non-limiting examples of fluorescent protein include Green Fluorescent Protein (GFP), Yellow Fluorescent Protein (YFP), Red Fluorescent Protein (RFP), Sirius, excitable blue fluorescent protein (EBFP2), cyan fluorescent protein (CFP), Cerulean, excitable green fluorescent protein (EGFP), excitable yellow fluorescent protein (EYFP), mOrange, mCherry, mPlum, NIR, iRFP, EosFP, PamCherry, Dronpa, Dreiklang, asFP595, mMaple, mGeo, mEos2, Dendra2, psCFP2, and 2,3,5,6-tetracarbazole-4-cyano-pyridine (CPy).

In some embodiments, florescence is achieved via the inclusion of at least one fluorescent nanoparticle associated with the delivery vehicle or the benchmark construct. In some embodiments, the fluorescent nanoparticle may be, but is not limited to, carbon dots, graphene quantum dots, gold nanorods, polymer-based nanoparticles, aggregation-induced emission dots, Conjugated Polymer nanoparticles (CP-dots), Gold nanospheres, Gold nano shells, Gold nanocages, and AIE pheromone.

In some embodiments, florescence is achieved via inclusion of at least one fluorescent lipid associated with or included in the delivery vehicle. In some embodiments, the fluorescent lipid may be, but is not limited to, DiR, DiD, DiO, and DiI, other members of the Di series of phospholipids, Bodipy, and FL-Sphingomyelin.

In some embodiments, florescence is achieved via the inclusion of at least one luciferase in or associated with the delivery vehicle. In some embodiments, at least one luciferase protein is encoded in the benchmark construct or the benchmark construct comprises the luciferase. Non-limiting examples of the types of luciferase which may be used include Renilla luciferase, Gaussia luciferase, Nanoluc luciferase, Firefly luciferase, and Click Beetle luciferases.

In some embodiments, florescence is achieved via inclusion of β-galactosidase (β-gal) associated with or included in the delivery vehicle. In some embodiments, at least one β-galactosidase (β-gal) protein is encoded in the benchmark construct or the benchmark construct comprises β-galactosidase (β-gal).

In some embodiments, florescence is achieved via inclusion of at least one quencher molecule associated with or included in the delivery vehicle. In some embodiments, florescence is achieved via inclusion of at least one quencher molecule associated with or encoded by the benchmark construct. Non-limiting examples of quencher molecules include dimethylaminophenylazobenzoic acid (DABCYL), QSY 7, Cu(II) ion, Dabcyl, QSY 35, BHQ-0, Eclipse, BHQ-1, QSY 9, BHQ-2, ElleQuencher, Iowa Black, QSY 21, and BHQ-3.

xii. Tracking System: Fluorophores and Radioactive Phosphates

In some embodiments, the at least one tracking system comprises an identifier sequence or moieties that is a fluorophore or radioactive phosphate.

In some embodiments, the at least one tracking system comprises the inclusion of at least one fluorophore associated with or included in the delivery vehicle. In some embodiments, the at least one tracking system comprises the inclusion of at least one fluorophore associated with, encoded in or included in the benchmark construct. Non-limiting examples of fluorophores includes quantum dot and organic small molecule.

In some embodiments, the at least one tracking system comprises the inclusion of at least one quantum dot associated with or included in the delivery vehicle. In some embodiments, the at least one tracking system comprises the inclusion of at least one quantum dot associated with, encoded in or included in the benchmark construct. Non-limiting examples of quantum dots include CdSe/ZnS, CdTe/ZnS, CdTe/CdSe, CdSe/ZnTe, CdSe/CdTe/ZnSe, nAs/ZnSe, InAs/CdSe, InAs/InP, Cu:InP/ZnSe, InAsxP1-x/InP/ZnSe, CdS/CdSe, ZnSe/CdSe, ZnSe/InP/ZnS, ZnSe/InP/ZnS, CdTe/ZnSe, QD585, and QD655.

In some embodiments, the at least one tracking system comprises the inclusion of at least one organic small molecule associated with or included in the delivery vehicle. In some embodiments, the at least one tracking system comprises the inclusion of at least one organic small molecule associated with, encoded in or included in the benchmark construct. Non-limiting examples of organic small molecules include classes of Coumarins, Naphthalimides, Fluoresceins and rhodamines derivatives, BODIPY, Cyanines, xanthenes, oxazines, Oligothiophenes, and Phthalocyanine derivatives (PcDer). In some embodiments, the at least one organic small molecule may be selected from, but is not limited to, 7-dialkyl-amino-4-trifluoromethyl coumarin, rhodamine B, Coumarin 314, Lucifer Yellow CH, florescein, rhodamine 123, BODIPY FL NHS ester, Cy5, Rhodamine 6G, Silicon-rhodamine (SiR), Cy3, Cy5.5, Cy7, Cy2, ATT0655, ATTO680, ATTO700, Nitrobenzoxadiazole (NBD), 1,6-diphenyl-1,3,5-hexatriene (DPH), ABBERIOR™, ALEXA FLUOR™, ATTO™, DYLIGHT FLUOR™, ALEXA FLUOR 647™, and TOPFLUOR™

In some embodiments, the at least one tracking system comprises the inclusion of at least one imaging contrast agent associated with or included in the delivery vehicle. In some embodiments, the at least one tracking system comprises the inclusion of at least one imaging contrast agent associated with, encoded in or included in the benchmark construct. Non-limiting examples of imaging contrast agents include gadolinium-based small molecules, gadolinium-encapsulated liposomes, manganese-based small molecules, and iron oxide nanoparticles.

In some embodiments, the at least one tracking system comprises the inclusion of at least one radiolabel associated with or included in the delivery vehicle. In some embodiments, the at least one tracking system comprises the inclusion of at least one radiolabel associated with, encoded in or included in the benchmark construct. Non-limiting examples of radiolabels include ¹¹¹In, ^{99m}Tc, ¹³N, ⁶⁸Ga, ¹⁸F, ⁶⁴Cu, ⁸⁶Y, ⁷⁶Br, ⁸⁹Zr, ⁷²As, ¹²⁴J, ⁷⁴As, fluorine-18, gallium-68, nitrogen-13, copper-64, bromine-76, iodine-125, arsenic-74, carbon-11, iodine-131, ¹⁵³Sm, ¹⁷⁷Lu, ¹⁸⁶Re, ¹⁸⁸Re, ¹⁹⁸Au, and ²²⁵Ac.

In some embodiments, the at least one tracking system comprises the inclusion of at least one biotin associated with or included in the delivery vehicle.

In some embodiments, the at least one tracking system comprises the inclusion of at least one digoxygenin associated with or included in the delivery vehicle.

In some embodiments, the at least one tracking system comprises the inclusion of at least one dinitrophenyl (DNP) associated with or included in the delivery vehicle.

In some embodiments, the at least one tracking system comprises the inclusion of at least one Fluorescein associated with or included in the delivery vehicle.

In some embodiments, the at least one tracking system comprises the inclusion of at least one fucose associated with or included in the delivery vehicle.

In some embodiments, the at least one tracking system comprises the inclusion of at least one amine associated with or included in the delivery vehicle.

In some embodiments, the at least one tracking system comprises the inclusion of at least one Texas Red® associated with or included in the delivery vehicle.

In some embodiments, the at least one tracking system comprises the inclusion of at least one biotin associated with, encoded in or included in the benchmark construct.

In some embodiments, the at least one tracking system comprises the inclusion of at least one digoxygenin associated with, encoded in or included in the benchmark construct.

In some embodiments, the at least one tracking system comprises the inclusion of at least one dinitrophenyl (DNP) associated with, encoded in or included in the benchmark construct.

In some embodiments, the at least one tracking system comprises the inclusion of at least one Fluorescein associated with, encoded in or included in the benchmark construct.

In some embodiments, the at least one tracking system comprises the inclusion of at least one fucose associated with, encoded in or included in the benchmark construct.

In some embodiments, the at least one tracking system comprises the inclusion of at least one amine associated with, encoded in or included in the benchmark construct.

In some embodiments, the at least one tracking system comprises the inclusion of at least one Texas Red® associated with, encoded in or included in the benchmark construct.

In some embodiments, the at least one tracking system comprises the inclusion of at least one reporter sequence or protein associated with or included in the delivery vehicle. In some embodiments, the at least one tracking system comprises the inclusion of at least one reporter sequence or protein associated with, encoded in or included in the benchmark construct. Non-limiting examples of reporter sequence or protein include eGFP, luciferase, gene editor (e.g. cas9 edit, DNA readout), ox-40, beta6 integrin, CD45, a surface marker with a HA tag, flag tag with or without a TEV protease site, or any combination thereof.

In some embodiments, the at least one tracking system comprises the inclusion of at least one functional sequence or protein associated with or included in the delivery vehicle. In some embodiments, the at least one tracking system comprises the inclusion of at least one functional sequence or protein associated with, encoded in or included in the benchmark construct. Non-limiting examples of functional sequence or protein include fluorescent protein, a surface protein, Cre-Recombinase, CRISPR/CAS system, surface protein with an epitope tag (e.g., HA, FLAG, etc.) or any combination thereof.

In some embodiments, the at least one tracking system comprises the inclusion of at least one functional sequence or protein that comprises a protease cleavage site (e.g., TEV) which may be associated with or included in the delivery vehicle. In some embodiments, the at least one tracking system comprises the inclusion of at least one functional sequence or protein that comprises a protease cleavage site (e.g., TEV) which may be associated with, encoded in or included in the benchmark construct.

In some embodiments, the at least one tracking system comprises the inclusion of at least one functional sequence or protein that comprises an affinity tag (e.g. 3xHA, FLAG, His) which may be associated with or included in the delivery vehicle. In some embodiments, the at least one tracking system comprises the inclusion of at least one functional sequence or protein that comprises an affinity tag (e.g. 3xHA, FLAG, His) which may be associated with, encoded in or included in the benchmark construct.

IV. PHARMACEUTICAL COMPOSITION AND ROUTE OF ADMINISTRATION a. Pharmaceutical Compositions and Formulations

The originator constructs, benchmark constructs, and targeting systems can be formulated using one or more excipients to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the cargo and/or payload.

Formulations can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers either to an originator construct or benchmark construct with a payload region or cargo or payload as described herein.

Formulations of the originator constructs, benchmark constructs, and targeting systems and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, or at least 80% (w/w) active ingredient.

b. Excipients and Diluents

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

c. Inactive Ingredients

In some embodiments, formulations described herein may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present invention may be approved by the US Food and Drug Administration (FDA).

In one embodiment, the formulations described herein comprise at least one inactive ingredient such as, but not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl.Alpha.-Ionone; Almond Oil; Alpha-Terpineol; Alpha-Tocopherol; Alpha-Tocopherol Acetate, D1-; Alpha-Tocopherol, D1-; Aluminum Acetate; Aluminum Chlorhydroxy Allantoinate; Aluminum Hydroxide; Aluminum Hydroxide-Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C₂₀-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, D1-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate-Butyl Methacrylate-Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, D1-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor FIG. 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O F1-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, D1-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa.S); Hypromellose 2910 (15000 Mpa.S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate-Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, D1-; Lactic Acid, L-; Lactobionic Acid; Lactose; Lactose Monohydrate; Lactose, Hydrous; Laneth; Lanolin; Lanolin Alcohol-Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa.S); Methylcelluloses; Methylchloroisothiazolinone; Methylene Blue; Methylisothiazolinone; Methylparaben; Microcrystalline Wax; Mineral Oil; Mono and Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy)Propane Anhydride):Sebacic Acid; Poly(Dimethylsiloxane/Methylvinylsiloxane/Methylhydrogensiloxane) Dimethylvinyl Or Dimethylhydroxy Or Trimethyl Endblocked; Poly(D1-Lactic-Co-Glycolic Acid), (50:50; Poly(D1-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene-Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f, Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; R^{a}-2397; R^{a}-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive,Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether.Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, D1-; Tenox; Tenox-2; Tert-Butyl Alcohol; Tert-Butyl Hydroperoxide; Tert-Butylhydroquinone; Tetrakis(2-Methoxyisobutylisocyanide)Copper(I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

In some embodiments, formulations disclosed herein may include cations or anions. The formulations include metal cations such as, but not limited to, Zn2+, Ca2+, Cu2+, Mn2+, Mg+ and combinations thereof. As a non-limiting example, formulations may include polymers and complexes with a metal cation.

Formulations of the invention may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

d. Routes of Administration

The originator constructs, benchmark constructs, and targeting systems described herein may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

In some embodiments, compositions may be administered in a way which allows them to cross the blood-brain barrier, vascular barrier, or other epithelial barrier. The originator constructs, benchmark constructs, and targeting systems may be administered in any suitable form, either as a liquid solution or suspension, as a solid form suitable for liquid solution or suspension in a liquid solution. The originator constructs, benchmark constructs, and targeting systems may be formulated with any appropriate and pharmaceutically acceptable excipient.

In some embodiments, the originator constructs, benchmark constructs, and targeting systems may be delivered to a subject via a single route administration.

In some embodiments, the originator constructs, benchmark constructs, and targeting systems may be delivered to a subject via a multi-site route of administration. A subject may be administered at 2, 3, 4, 5, or more than 5 sites.

In some embodiments, a subject may be administered the originator constructs, benchmark constructs, and targeting systems using a bolus infusion.

In some embodiments, a subject may be administered originator constructs, benchmark constructs, and targeting systems using sustained delivery over a period of minutes, hours, or days. The infusion rate may be changed depending on the subject, distribution, formulation or another delivery parameter.

In some embodiment, the originator constructs, benchmark constructs, and targeting systems may be delivered by intramuscular delivery route. Non-limiting examples of intramuscular administration include an intravenous injection or a subcutaneous injection.

In some embodiments, the originator constructs, benchmark constructs, and targeting systems may be delivered by oral administration. Non-limiting examples of oral delivery include a digestive tract administration and a buccal administration.

In some embodiments, the originator constructs, benchmark constructs, and targeting systems may be delivered by intraocular delivery route. A non-limiting example of intraocular delivery include an intravitreal injection.

In some embodiment, the originator constructs, benchmark constructs, and targeting systems may be delivered by intranasal delivery route. Non-limiting examples of intranasal delivery include nasal drops or nasal sprays.

In some embodiments, the originator constructs, benchmark constructs, and targeting systems may be administered to a subject by peripheral injections. Non-limiting examples of peripheral injections include intraperitoneal, intramuscular, intravenous, conjunctival, or joint injection.

In some embodiments, the originator constructs, benchmark constructs, and targeting systems may be delivered by injection into the cerebrospinal fluid. Non-limiting examples of delivery to the cerebrospinal fluid include intrathecal and intracerebroventricular administration.

In some embodiments, the originator constructs, benchmark constructs, and targeting systems may be delivered by systemic delivery. As a non-limiting example, the systemic delivery may be by intravascular administration.

In some embodiments, the originator constructs, benchmark constructs, and targeting systems may be administered to a subject by intracranial delivery.

In some embodiments, the originator constructs, benchmark constructs, and targeting systems may be administered to a subject by intraparenchymal administration.

In some embodiments, the originator constructs, benchmark constructs, and targeting systems may be administered to a subject by intramuscular administration.

In some embodiments, the originator constructs, benchmark constructs, and targeting systems are administered to a subject and transduce muscle of a subject. As a non-limiting example, the originator constructs, benchmark constructs, and targeting systems are administered by intramuscular administration.

In some embodiments, the originator constructs, benchmark constructs, and targeting systems may be administered to a subject by intravenous administration.

In some embodiments, the originator constructs, benchmark constructs, and targeting systems may be administered to a subject by subcutaneous administration.

In some embodiments, the originator constructs, benchmark constructs, and targeting systems may be administered to a subject by topical administration.

In some embodiments, the originator constructs, benchmark constructs, and targeting systems may be delivered by more than one route of administration.

i. Injectable and Parenteral Administration

In some embodiments, pharmaceutical compositions and/or formulations described herein may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues. ii. Topical or Transdermal Administration

In some embodiments, pharmaceutical compositions and/or formulations described herein may be formulated for administration topically. The skin may be an ideal target site for delivery as it is readily accessible. Three routes are commonly considered to deliver pharmaceutical compositions and/or formulations described herein to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions).

In some embodiments, pharmaceutical compositions and/or formulations described herein may be delivered using a variety of dressings (e.g., wound dressings) or bandages (e.g., adhesive bandages) for conveniently and/or effectively carrying out methods described herein. Typically dressing or bandages may comprise sufficient amounts of pharmaceutical compositions and/or formulations described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, contemplated herein is the use of transdermal patches, which often have the added advantage of providing controlled delivery of pharmaceutical compositions and/or formulations described herein to the body. Such dosage forms may be prepared, for example, by dissolving and/or dispensing pharmaceutical compositions and/or formulations described herein in the proper medium. Alternatively, or additionally, rates may be controlled by either providing rate controlling membranes and/or by dispersing pharmaceutical compositions and/or formulations described herein in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

iii. Ophthalmic or Otic Administration

In some embodiments, pharmaceutical compositions and/or formulations described herein may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations which are useful include those which comprise active ingredients in microcrystalline form and/or in liposomal preparations. Subretinal inserts may also be used as forms of administration.

iv. Oral Administration

In some embodiments, pharmaceutical compositions and/or formulations described herein may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

v. Depot Administration

In some embodiments, p pharmaceutical compositions and/or formulations described herein are formulated in depots for extended release.

In some embodiments, pharmaceutical compositions and/or formulations described herein are spatially retained within or proximal to target tissues. Provided are methods of providing pharmaceutical compositions and/or formulations described herein to target tissues of mammalian subjects by contacting target tissues (which comprise one or more target cells) with pharmaceutical compositions and/or formulations described herein under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions and/or formulations described herein that enter one or more target cells. For example, at least 1%, 5%, 10%, 20%, 30%, 40%, 50%, 60%, 70%, 80%, 85%0, 90%, 95%, 96%, 97%, 98%, 99%, 99.9% 99.99%, or greater than 99.99% of pharmaceutical compositions and/or formulations described herein administered to subjects are present intracellularly at a period of time following administration. For example, intramuscular injection to mammalian subjects may be performed using aqueous compositions comprising an active ingredient and one or more transfection reagents, and retention is determined by measuring the amount of active ingredient present in muscle cells.

In some embodiments, provided are methods for delivering pharmaceutical compositions and/or formulations described herein to target tissues of mammalian subjects, by contacting target tissues (comprising one or more target cells) with pharmaceutical compositions and/or formulations described herein under conditions such that they are substantially retained in such target tissues. Pharmaceutical compositions and/or formulations described herein comprise enough active ingredient such that the effect of interest is produced in at least one target cell. In some embodiments, pharmaceutical compositions and/or formulations described herein generally comprise one or more cell penetration agents, although “naked” formulations (such as without cell penetration agents or other agents) are also contemplated, with or without pharmaceutically acceptable carriers.

vi. Pulmonary Administration

In some embodiments, pharmaceutical compositions and/or formulations described herein may be prepared, packaged, and/or sold in formulations suitable for pulmonary administration. In some embodiments, such administration is via the buccal cavity. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

vii. Intranasal, Nasal or Buccal Administration

In some embodiments, pharmaceutical compositions and/or formulations described herein may be administered nasally and/or intranasal. In some embodiments, formulations described herein useful for pulmonary delivery may also be useful for intranasal delivery. In some embodiments, formulations for intranasal administration comprise a coarse powder comprising the active ingredient and having an average particle from about 0.2 μm to 500 μm. Such formulations are administered in the manner in which snuff is taken, e.g., by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

viii. Rectal and Vaginal Administration

In some embodiments, pharmaceutical compositions and/or formulations described herein may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### V. Target Area, Tissue or Cell for Delivery

The delivery of nucleic acid sequences, polypeptides or peptides and formulations thereof can be localized to specific target areas, tissues or cells using the methods and targeted delivery systems described herein.

a. Tumors

In some embodiments, the delivery of nucleic acid sequences, polypeptides or peptides and formulations thereof can be localized to a tumor. The tumor may be a benign tumor or a malignant tumor.

In some embodiments, the delivery of nucleic acid sequences, polypeptides or peptides and formulations thereof is localized to a connective tissue tumor such as, but not limited to, adult fibrous tissue, embryonic (myxomatous) fibrous tissue, fat tissue, cartilage, bone, and notochord. As a non-limiting example, the tumor is a benign tumor called fibroma located in adult fibrous tissue. As a non-limiting example, the tumor is a malignant tumor called fibrosarcoma located in adult fibrous tissue. As a non-limiting example, the tumor is a benign tumor called myxoma located in embryonic fibrous tissue. As a non-limiting example, the tumor is a malignant tumor called myxosarcoma located in embryonic fibrous tissue. As a non-limiting example, the tumor is a benign tumor called lipoma located in fat tissue. As a non-limiting example, the tumor is a malignant tumor called liposarcoma located in fat tissue. As a non-limiting example, the tumor is a benign tumor called chondroma located in cartilage. As a non-limiting example, the tumor is a malignant tumor called chondrosarcoma located in cartilage. As a non-limiting example, the tumor is a benign tumor called osteoma located in bone. As a non-limiting example, the tumor is a malignant tumor called osteosarcoma located in bone. As a non-limiting example, the tumor is a malignant tumor called chordoma located in notochord. As a non-limiting example, the tumor is a benign tumor called fibrous histiocytoma located in connective tissue. As a non-limiting example, the tumor is a malignant tumor called malignant fibrous histiocytoma located in connective tissue.

In some embodiments, the delivery of nucleic acid sequences, polypeptides or peptides and formulations thereof is localized to endothelium and/or mesothelium tumor tissue such as, but not limited to, blood vessels, lymph vessels and mesothelium. As a non-limiting example, the tumor is a benign tumor called hemangioma located in blood vessels. As a non-limiting example, the tumor is a benign tumor called hemangiopericytoma located in blood vessels. As a non-limiting example, the tumor is a malignant tumor called hemangiosarcoma located in blood vessels. As a non-limiting example, the tumor is a malignant tumor called angiosarcoma located in blood vessels. As a non-limiting example, the tumor is a benign tumor called lymphangioma located in lymph vessels. As a non-limiting example, the tumor is a malignant tumor called lymphangiosarcoma located in lymph vessels. As a non-limiting example, the tumor is a malignant tumor called mesothelioma located in the mesothelium.

In some embodiments, the delivery of nucleic acid sequences, polypeptides or peptides and formulations thereof is localized to blood and lymphoid cell tissue such as, but not limited to, hematopoietic cells and lymphoid tissue. As a non-limiting example, the tumor is a benign tumor called preleukemias located in hematopoietic cells. As a non-limiting example, the tumor is a benign tumor called myeloproliferative disorders located in hematopoietic cells. As a non-limiting example, the tumor is a malignant tumor called leukemia located in hematopoietic cells. As a non-limiting example, the tumor is a benign tumor called plasmacytosis located in lymphoid tissue. As a non-limiting example, the tumor a malignant tumor called plasmacytoma located in lymphoid tissue. As a non-limiting example, the tumor a malignant tumor called multiple myeloma located in lymphoid tissue. As a non-limiting example, the tumor a malignant tumor called Hodgkin lymphoma located in lymphoid tissue. As a non-limiting example, the tumor a malignant tumor called Non-Hodgkin lymphoma located in lymphoid tissue.

In some embodiments, the delivery of nucleic acid sequences, polypeptides or peptides and formulations thereof is localized to muscle tissue such as, but not limited to, smooth muscle and striated muscle. As a non-limiting example, the tumor is a benign tumor called Leiomyoma located in smooth muscle. As a non-limiting example, the tumor is a malignant tumor called leiomyosarcoma located in smooth muscle. As a non-limiting example, the tumor is a benign tumor called rhabdomyoma located in striated muscle. As a non-limiting example, the tumor is a malignant tumor called rhabdomyosarcoma located in striated muscle.

In some embodiments, the delivery of nucleic acid sequences, polypeptides or peptides and formulations thereof is located to epithelial tissue such as, but not limited to, stratified squamous tissue, glandular epithelium tissue (e.g., liver, kidney, bile duct), transitional epithelium tissue, placenta and testis. As a non-limiting example, the tumor is a benign tumor called papilloma located in stratified squamous. As a non-limiting example, the tumor is a benign tumor called seborrheic keratosis located in stratified squamous. As a non-limiting example, the tumor is a malignant tumor called squamous cell carcinoma located in stratified squamous tissue. As a non-limiting example, the tumor is a malignant tumor called epidermoid carcinoma located in stratified squamous tissue. As a non-limiting example, the tumor is a benign tumor called adenoma located in glandular epithelium tissue. As a non-limiting example, the tumor is a benign tumor called hepatic adenoma located in liver glandular epithelium tissue. As a non-limiting example, the tumor is a benign tumor called renal tubular adenoma located in kidney glandular epithelium tissue. As a non-limiting example, the tumor is a benign tumor called bile duct adenoma located in bile duct glandular epithelium tissue. As a non-limiting example, the tumor is a malignant tumor called adenocarcinoma located in glandular epithelium tissue. As a non-limiting example, the tumor is a malignant tumor called hepatoma located in liver glandular epithelium tissue. As a non-limiting example, the tumor is a malignant tumor called hepatocellular carcinoma located in liver glandular epithelium tissue. As a non-limiting example, the tumor is a malignant tumor called renal cell carcinoma located in kidney glandular epithelium tissue. As a non-limiting example, the tumor is a malignant tumor called hypernephroma located in kidney glandular epithelium tissue. As a non-limiting example, the tumor is a malignant tumor called cholangiocarcinoma located in bile duct glandular epithelium tissue. As a non-limiting example, the tumor is a benign tumor called transitional cell papilloma located in transitional epithelium tissue. As a non-limiting example, the tumor is a malignant tumor called transitional cell carcinoma located in transitional epithelium tissue. As a non-limiting example, the tumor is a benign tumor called hydatidiform mole located in the placenta. As a non-limiting example, the tumor is a malignant tumor called choriocarcinoma located in the placenta. As a non-limiting example, the tumor is a malignant tumor called seminoma located in the testis. As a non-limiting example, the tumor is a malignant tumor called embryonal cell carcinoma located in the testis.

In some embodiments, the delivery of nucleic acid sequences, polypeptides or peptides and formulations thereof is located to neural tissue such as, but not limited to, glial cells, nerve cells, meninges, and nerve sheath. As a non-limiting example, the tumor is a malignant tumor called glioma (grades I-III) located in glial cells. As a non-limiting example, the tumor is a malignant tumor called anaplastic glioma (grades I-III) located in glial cells. As a non-limiting example, the tumor is a malignant tumor called glioblastoma multiforme (grade IV) located in glial cells. As a non-limiting example, the tumor is a benign tumor called ganglioneuroma located in nerve cells. As a non-limiting example, the tumor is a malignant tumor called neuroblastoma located in nerve cells. As a non-limiting example, the tumor is a malignant tumor called medulloblastoma located in nerve cells. As a non-limiting example, the tumor is a benign tumor called meningioma located in meninges tissue. As a non-limiting example, the tumor is a malignant tumor called malignant meningioma located in meninges tissue. As a non-limiting example, the tumor is a benign tumor called schwannoma located in the nerve sheath. As a non-limiting example, the tumor is a benign tumor called neurilemmoma located in the nerve sheath. As a non-limiting example, the tumor is a benign tumor called neurofibroma located in the nerve sheath. As a non-limiting example, the tumor is a malignant tumor called malignant meningioma located in the nerve sheath. As a non-limiting example, the tumor is a malignant tumor called malignant schwannoma located in the nerve sheath. As a non-limiting example, the tumor is a malignant tumor called neurofibrosarcoma located in the nerve sheath.

In some embodiments, the delivery of nucleic acid sequences, polypeptides or peptides and formulations thereof is located to the Amine Precursor Uptake and Decarboxylation (APUD) System such as, but not limited to, pituitary tissue, parathyroid tissue, thyroid tissue, bronchial tissue, adrenalmedulla tissue, pancreas tissue, stomach and intestines, carotid body and chemo-receptor system tissue. The APUD system is a series of cells which have endocrine functions and secrete a variety of small amine or polypeptide hormones. As a non-limiting example, the tumor is a benign tumor called basophilic adenoma located in the pituitary tissue. As a non-limiting example, the tumor is a benign tumor called eosinophilic adenoma located in the pituitary tissue. As a non-limiting example, the tumor is a benign tumor called chromophobe adenoma located in the pituitary tissue. As a non-limiting example, the tumor is a benign tumor called parathyroid adenoma located in the parathyroid. As a non-limiting example, the tumor is a malignant tumor called parathyroid carcinoma located in the parathyroid. As a non-limiting example, the tumor is a benign tumor called c cell hyperplasia located in the thyroid tissue (C cells). As a non-limiting example, the tumor is a malignant tumor called medullary carcinoma of thyroid located in the thyroid tissue (C cells). As a non-limiting example, the tumor is a malignant tumor called bronchial carcinooid located in the bronchial lining (Kultschitzky cells). As a non-limiting example, the tumor is a malignant tumor called oat cells carcinoma located in the bronchial lining (Kultschitzky cells). As a non-limiting example, the tumor is a benign tumor called pheochromocytoma located in the adrenalmedulla. As a non-limiting example, the tumor is a malignant tumor called malignant pheochromocytoma located in the adrenalmedualla. As a non-limiting example, the tumor is a benign tumor called islet cell adenoma located in the pancreas. As a non-limiting example, the tumor is a benign tumor called insulinoma located in the pancreas. As a non-limiting example, the tumor is a benign tumor called gastrinoma located in the pancreas. As a non-limiting example, the tumor is a malignant tumor called islet cell carcinoma located in the pancreas. As a non-limiting example, the tumor is a benign tumor called carcinoid located in the stomach and intestines. As a non-limiting example, the tumor is a malignant tumor called malignant carcinoid located in the stomach and intestines. As a non-limiting example, the tumor is a benign tumor called chemodectoma located in the carotid body and chemo-receptor system. As a non-limiting example, the tumor is a benign tumor called paraganglioma located in the carotid body and chemo-receptor system. As a non-limiting example, the tumor a malignant tumor called malignant carcinoid located in the carotid body and chemo-receptor system. As a non-limiting example, the tumor a malignant tumor called malignant paraganglioma located in the carotid body and chemo-receptor system.

In some embodiments, the delivery of nucleic acid sequences, polypeptides or peptides and formulations thereof is located in neural crest-derived cells such as, but not limited to, pigment-producing cells (e.g., skin and eyes), schwann cells of the peripheral nervous system, and merkel cells in the squamous epithelium. As a non-limiting example, the tumor is a benign tumor called nevus located in pigment-producing cells such as the skin and eyes. As a non-limiting example, the tumor a malignant tumor called melanoma located in pigment-producing cells such as the skin and eyes. As a non-limiting example, the tumor is a benign tumor called schwannoma or neurilemmoma located in schwann cells of the peripheral nervous system. As a non-limiting example, the tumor is a malignant tumor called malignant schwannoma located in schwann cells of the peripheral nervous system. As a non-limiting example, the tumor is a malignant tumor called merkel cell neoplasm located in merkel cells in the squamous epithelium.

In some embodiments, the delivery of nucleic acid sequences, polypeptides or peptides and formulations thereof is located in breast tissue. As a non-limiting example, the tumor is a benign tumor called fibroadenoma. As a non-limiting example, the tumor is a malignant tumor called cystosarcoma phylloides.

In some embodiments, the delivery of nucleic acid sequences, polypeptides or peptides and formulations thereof is located in renal anlage tissue. As a non-limiting example, the tumor is a malignant tumor called Wilms tumor.

In some embodiments, the delivery of nucleic acid sequences, polypeptides or peptides and formulations thereof is located in ovary tissue.

In some embodiments, the delivery of nucleic acid sequences, polypeptides or peptides and formulations thereof is located in testis tissue.

In some embodiments, the delivery of nucleic acid sequences, polypeptides or peptides and formulations thereof is located in germ cell tumor tissue. Non-limiting examples of germ cell tumors including seminoma, dysgerminoma, choriocarcinoma, embryonal carcinoma, endodermal sinus tumor, and teratocarcinoma.

In some embodiments, the delivery of nucleic acid sequences, polypeptides or peptides and formulations thereof is located in the connective tissue stroma. Non-limiting examples of these tumors are Sertoli-Leydig cell tumors, arrhenoblastoma, granulose-theca cell tumors, hilar cell tumors, lipid cell tumors.

b. Organs

In some embodiments, the delivery of nucleic acid sequences, polypeptides or peptides and formulations thereof can be localized to an organ. Non-limiting example of organs include the anal canal, arteries, ascending colon, bladder, bone marrow, brain, bronchi, bronchioles, bulbourethral glands, capillaries, cecum, cerebellum, cerebral hemispheres, cerebrum, cervix, choroid plexus, clitoris, cranial nerves, descending colon, diencephalon, duodenum, ear, enteric nervous system, epididymis, esophagus, external reproductive organs, fallopian tubes, gallbladder, ganglia, gustatory, gut-associated lymphoid tissue, heart, ileum, internal reproductive organs, interstitium, jejunum, joints, kidneys, large intestine, larynx, ligaments, liver, lungs, lymph node, lymphatic vessel, mammary glands, medulla oblongata, mesentery, midbrain, mouth, muscles of breathing, nasal cavity, nerves, olfactory, ovaries, pancreas, parotid glands, penis, pharynx, placenta, pons, prostate, rectum, salivary glands, scrotum, seminal vesicles, sigmoid colon, skeleton, skin, small intestine, spinal nerves, spleen, stomach, subcutaneous tissue, sublingual glands, submandibular glands, teeth, tendons, testes, the brainstem, the spinal cord, the ventricular system, thymus, tongue, tonsils, trachea, transverse colon, ureter, urethra, uterus, vagina, vas deferens, veins, and vulva.

c. Tissues

In some embodiments, the delivery of nucleic acid sequences, polypeptides or peptides and formulations thereof can be localized to a tissue. Non-limiting example of adrenal medulla, adult fibrous tissue, blood vessels, bone, breast, bronchial lining, carotid body, cartilage, connective tissue, embryonic (myxomatous) fibrous tissue, epithelial, epithelium, fat, glandular epithelium (liver, kidney, bile duct), gonads, hematopoietic cells, lymph vessels, lymphoid tissue, meninges, mesothelium, muscle, nerve sheath, nervous, notochord, ovary, pancreas, parathyroid, pituitary, placenta, renal anlage, smooth muscle, stomach and intestines, stratified squamous, striated muscle, stroma, testis, thyroid, and transitional epithelium. As a non-limiting example, the tissue is connective tissue.

d. Cells

In some embodiments, the delivery of nucleic acid sequences, polypeptides or peptides and formulations thereof can be localized to a particular cell type. Non-limiting example of cells include adipocytes, adrenergic neural cells, alpha cell, amacrine cells, ameloblast, anterior lens epithelial cell, anterior/intermediate pituitary cells, apocrine sweat gland cell, astrocytes, auditory inner hair cells of organ of corti, auditory outer hair cells of organ of corti, b cell, bartholin's gland cell, basal cell (stem cell) of cornea, tongue, mouth, nasal cavity, distal anal canal, distal urethra, and distal vagina, basal cells of olfactory epithelium, basket cells, basophil granulocyte and precursors, beta cell, betz cells, bone marrow reticular tissue fibroblasts, border cells of organ of corti, boundary cells, bowman's gland cell, brown fat cell, brunner's gland cell, bulbourethral gland cell, bushy cells, c cells, cajal-retzius cells, cardiac muscle cell, cardiac muscle cells, cartwheel cells, cells of the zona fasciculata produce glucocorticoids, cells of the zona glomerulosa produce mineralocorticoids, cells of the zona reticularis produce androgens, cells of the adrenal cortex, cementoblast, centroacinar cell, ceruminous gland cell in ear, chandelier cells, chemoreceptor glomus cells of carotid body cell, chief cell, cholinergic neurons, chromaffin cells, club cell, cold-sensitive primary sensory neurons, connective tissue macrophage (all types), corneal fibroblasts (corneal keratocytes), corpus luteum cell of ruptured ovarian follicle secreting progesterone, cortical hair shaft cell, corticotropes, crystallin-containing lens fiber cell, cuticular hair shaft cell, cytotoxic t cell, d cell, delta cell, dendritic cell, double-bouquet cells, duct cell, eccrine sweat gland clear cell, eccrine sweat gland dark cell, efferent ducts cell, elastic cartilage chondrocyte, endothelial cells, enteric glial cells, enterochromaffin cell, enterochromaffin-like cell, enteroendocrine cell, eosinophil granulocyte and precursors, ependymal cells, epidermal basal cell, epidermal langerhans cell, epididymal basal cell, epididymal principal cell, epithelial reticular cell, epsilon cell, erythrocyte, fibrocartilage chondrocyte, fork neurons, foveolar cell, g cell, gall bladder epithelial cell, germ cells, gland of littre cell, gland of moll cell in eyelid, glial cells, golgi cells, gonadal stromal cells, gonadotropes, granule cells, granulosa cell, granulosa lutein cells, grid cells, and head direction cells. In some embodiments, the at least one cell type comprise cancerous cells. In some embodiments, the at least one cell type comprise non-cancerous cells. In some embodiments, the at least one cell type comprise both cancerous and non-cancerous type. In some embodiments, the cancerous state of the at least one cell type is unknown.

e. Physiological Systems

In some embodiments, the delivery of nucleic acid sequences, polypeptides or peptides and formulations thereof can be localized to a physiological system. Non-limiting example of physiological system include the auditory, cardiovascular, central nervous system, chemo-receptor system, circulatory, digestive, endocrine, excretory, exocrine, genital, integumentary, lymphatic, muscular, musculoskeletal, nervous, peripheral nervous system, renal, reproductive, respiratory, urinary, and visual systems.

### VI. Methods of Detection and Analysis

Detection of the tropism discovery platform including the targeting systems (e.g., candidate targeting system and validated targeting system) may be carried out through a variety of techniques (e.g., detection techniques or analysis techniques, both of which are used interchangeably herein) which can be selected based on the tracking system used.

In some embodiments, the targeting systems described herein is detected utilizing a nuclear imaging technique. Nuclear imaging techniques, as used herein, are meant to encompass any imaging, detection, couniting, or sorting technique that utilizes radioactive emissions, ether emitted from the subject or an external source. Without limitation, nuclear imaging techniques may include X-ray, magnetic resonance imaging (MRI) including functional magnetic resonance imaging (fMRI) and nuclear magnetic resonance imaging, computed tomography (CT), positron emission tomography (PET), single-photon emission computed tomography (SPECT), absorption imaging, or any combination thereof. The general principles and procedures of these approaches are known in the art, see P6rez-Medina, et. al. Nuclear imaging approaches facilitating nanomedicine translation. Advanced Drug Delivery Reviews 154-155 (2020) 123-141, the contents of which are herein incorporated by reference in their entirety as it relates to nuclear imaging techniques.

In some embodiments, detection of the targeting systems described herein in a subject may be performed utilizing MRI techniques. This approach may be carried out by any method known or discovered. While not wishing to be bound by theory, MRI utilizes the detection of certain nuclide spin characteristics. In some embodiments, MRI may be used as a non-invasive detection technique along with the targeting systems described herein that comprises an MRI contrast agent such as gadolinium-based small molecules, manganese-based small molecules, iron oxide nanoparticles, ¹⁹F-based compounds, and any combination thereof. MRI techniques may, as an example, allow for a detection of the targeting systems in specific organs and tissues of a subject in vivo, as well as changes in those distributions over time.

In some embodiments, detection of the targeting systems described herein in a subject may be performed utilizing CT techniques. This approach may be carried out by any method known or discovered. While not wishing to be bound by theory, CT utilizes the interaction of X-ray photons with matter, CT may be used as a non-invasive detection technique along with the targeting systems that comprise an CT contrast agent such as a gold high-density lipoprotein nanoparticle (Au-HDL). CT techniques may, as an example, allow for a detection of the targeting systems in specific organs and tissues of a subject in vivo, as well as changes in those distributions over time.

In some embodiments, detection of the targeting systems described herein in a subject may be performed utilizing PET techniques. This approach may be carried out by any method known or discovered. While not wishing to be bound by theory, PET utilize detection of photon emission from exogenously administered radiological substances, e.g., radiotracers. Principally, PET scanners detect the two photons emitted in opposite directions after positron-electron annihilation (the coincidence event). PET may be used as either an invasive or non-invasive detection technique along with the targeting systems that comprise an appropriate radiolabel such as 111In, 99mTc, 13N, 68Ga, 18F, 64Cu, 86Y, 76Br, 89Zr, 72As, 1241, 74As, fluorine-18, gallium-68, nitrogen-13, copper-64, bromine-76, iodine-125, arsenic-74, carbon-11, iodine-131, 153Sm, 177Lu, 186Re, 188Re, 198Au, and 225Ac. These labels may be conjugated to either the structural elements, the cargo components, or both. PET scans may be performed to detect distribution of the targeting systems either on the subject in vivo, including changes in those distributions over time, or on excised samples of the subject. PET techniques may allow for detection of the targeting systems in a subject from the organ/tissue level down to the cell type level. Some PET techniques may allow for detection of the targeting systems at the intracellular level.

In some embodiments, detection of the targeting systems described herein in a subject may be performed utilizing SPECT techniques. This approach may be carried out by any method known or discovered. While not wishing to be bound by theory, SPECT utilize detection of photon emission from exogenously administered radiological substances, e.g., radiotracers. Principally, SPECT scanners detect X-ray and gamma photons associated with nuclear state transitions. SPECT may be used as either an invasive or non-invasive detection technique along with the targeting systems that comprise an appropriate radiolabel such as 111In, 99mTc, 13N, 68Ga, 18F, 64Cu, 86Y, 76Br, 89Zr, 72As, 1241, 74As, fluorine-18, gallium-68, nitrogen-13, copper-64, bromine-76, iodine-125, arsenic-74, carbon-11, iodine-131, 153Sm, 177Lu, 186Re, 188Re, 198Au, and 225Ac. These labels may be conjugated to either the structural elements, the cargo components, or both. SPECT scans may be performed to detect distribution of the targeting systems either on the subject in vivo, including changes in those distributions over time, or on excised samples of the subject. SPECT techniques may allow for detection of the targeting systems in a subject from the organ/tissue level down to the cell type level. Some SPECT techniques may allow for detection of the targeting systems at the intracellular level.

In some embodiments, multiple nuclear imaging techniques may be used with the targeting systems comprising a single tracking system. In some embodiments, multiple nuclear imaging techniques may be used with the targeting systems comprising multiple tracking systems.

In some embodiments, the targeting systems described herein is detected utilizing an optical imaging technique. Optical imaging techniques, as used herein, are meant to encompass any imaging, detection, couniting, or sorting technique that utilizes light emissions and the special properties of photons, ether emitted from the subject or an external source. Without limitation, optical imaging techniques may include visible light microscopy, Raman spectroscopy, fluorescence microscopy, bioluminescence imaging (BLI), optical coherence tomography, or any combination thereof. The general principles and procedures of these approaches are known in the art, see Drummen. Fluorescent Probes and Fluorescence (Microscopy) Techniques Illuminating Biological andBiomedical Research. Molecules 2012, 17, 14067-14090, Boutorine, et. al. Fluorescent Probes for Nucleic Acid Visualization in Fixed and Live Cells. Molecules 2013, 18, 15357-15397, and Juskowiak. Nucleic acid-based fluorescentprobes and their analytical potential. Anal Bioanal Chem (2011) 399:3157-3176, the contents of which are herein incorporated by reference in their entirety as relates to optical imaging techniques.

In some embodiments, detection of the targeting systems described herein in a subject may be performed utilizing visible fluorescence microscopy techniques. Fluorescence microscopy techniques include a wide range of techniques known in the art including without limitation confocal fluorescence microscopy, fluorescence reflectance imaging, fluorescence molecular tomographic imaging, and Forster Resonance Energy Transfer (FRET). In general, all fluorescence microscopy techniques utilize detection of light emitted from endogenously present or exogenously administered fluorescent compounds, e.g., compounds which absorb light or other electromagnetic radiation and re-emits it at longer wavelengths. Fluorescence microscopy techniques may be used as either an invasive or non-invasive detection technique along with the targeting systems that comprise at least one tracking system which comprises an appropriate fluorescent compound. Without limitation, such fluorescent compounds may include Green Florescent Protein, Yellow Florescent Protein, Red Florescent Protein, Sirius, EBFP2, CFP, Cerulean, EGFP, EYFP, mOrange, mCherry, mPlum, NIR, iRFP, EosFP, PamCherry, Dronpa, Dreiklang, asFP595, mMaple, mGeo, mEos2, Dendra2, psCFP2, 2,3,5,6-tetracarbazole-4-cyano-pyridine (CPy), florescent nanoparticles, or florescent lipids, fluorescein, TAMRA, Cy dyes, Texas red, HEX, JOE, Oregon green, rhodamine 6 G, coumarin, pyrene, DiOC6 (3,3′-dihexyloxacarbocyanine iodide), or any combination thereof. In some embodiments, a targeting system for detection with fluorescence microscopy will comprise at least one fluorophore which may include, without limitation, a quantum dot, a Coumarins, a Naphthalimide, a Fluorescein, a BODIPY, a Cyanine, a xanthene, an oxazine, an Oligothiophenes, and a Phthalocyanine derivative (PcDer). These fluorescence compounds may be incorporated into the structure of the targeting systems, loaded as a cargo or payload, expressed as the product of a cargo or payload, or any combination thereof. Fluorescence microscopy techniques may be performed to detect distribution of the targeting systems either on the subject in vivo, including changes in those distributions over time, or on excised samples of the subject. Fluorescence microscopy techniques may allow for detection of the targeting systems in a subject from the organ/tissue level down to the cell type level. Some fluorescence microscopy techniques may allow for detection of the targeting systems at the intracellular level. In some embodiments, fluorescence microscopy techniques may be used to sort samples of cells post administration utilizing Fluorescence-activated Cell Sorting (FACS).

In some embodiments, detection of the targeting systems in a subject may be performed utilizing bioluminescence imaging (BLI) techniques. This approach may be carried out by any method known or discovered. While not wishing to be bound by theory, BLI imaging utilizes exogenously supplied compounds which emit light as a product of a chemical reaction under physiological condition. These emissions may be detected through various techniques of light and fluorescence microscopy. In some embodiments, BLI techniques may be used in conjunction with targeting systems which comprise bioluminescent compounds. Such compounds may be incorporated into nanoparticles or as the cargo or payload for expression post-delivery. In some embodiments, bioluminescent compounds may include, but are not limited to, luciferases including Renilla luciferase, Gaussia luciferase, Nanoluc luciferase, Firefly luciferase, Click Beetle luciferases, or any combination thereof. BLI techniques may be performed to detect distribution of the tropism discovery platform either on the subject in vivo, including changes in those distributions over time, or on excised samples of the subject. BLI may allow for detection of the targeting systems in a subject from the organ/tissue level down to the cell type level. Some BLI techniques may allow for detection of the targeting systems at the intracellular level. In some embodiments, BLI techniques may include quantifying luciferase expression from different organs with an in vivo imaging system (IVIS).

In some embodiments, detection of the targeting systems described herein may be performed utilizing nucleotide sequencing techniques. Nucleotide sequencing techniques maybe used to detect the presence of a known sequence of nucleotides, such as an identifier (e.g., barcode) sequence, in a sample. Non-limiting examples of nucleotide sequencing techniques which may be used to detect the targeting systems include high throughput sequencing, PCR, deep sequencing, and any combination thereof.

In some embodiments, detection of the targeting systems described herein may be performed by detecting the product of a tracking system which comprises a functional polynucleotide (e.g., DNA, mRNA, or oRNA) coding for a known peptide sequence or protein (e.g., a reporter sequence). In some embodiments, the functional polynucleotide may comprise a sequence which codes for a unique non-functional polypeptide sequence (e.g., a peptide, a polypeptide, a protein). In some embodiments, the reporter sequence may comprise a β-galactosidase (β-gal) sequence. In some embodiments, the reporter sequence may comprise a eGFP, luciferase, gene editor (e.g. cas9 edit, DNA readout), ox-40, beta6 integrin, CD45, a surface marker with (3x)-HA tag, (3x)-flag tag (with or without) a TEV protease site, or any combination thereof. In some embodiments, the reporter sequence may comprise a luciferase or fluorescent compound sequence. In some embodiments, the expression of the functional sequence, and by extension the presence of the targeting systems may be performed by any technique disclosed previously. In some embodiments, detecting the product of a tracking system which comprises a reporter sequence may be performed using any method known or discovered to detect products of expression. Such techniques include, but are not limited to, liquid/gas chromatography, mass spectrometry, light spectrometry (absorbance), gel electrophoresis, quantitative enzyme-linked immunosorbent assays (ELISA), Western blotting, dot blotting, Northern Blotting, protein immunostaining, protein immunoprecipitation, or any combination thereof.

In some embodiments, detection of the targeting systems described herein may be performed by utilizing detections systems chosen to match especially designed tracking systems. As a non-limiting example, the targeting systems described herein may be detected by electron microscopy, thermal imaging, ultrasound imaging, photoacoustic imaging, lab assays, and any combination thereof.

In some embodiments, detection of the targeting systems described herein may be performed by utilizing cell sorting techniques, including but not limited to, magnetic beads, flow cytometry, cleavage of peptide with LC-MS/MS, Fluorescence-activated Cell Sorting (FACS), or any combination thereof, combined with tracking system nanoparticles comprising components recognized by the cell sorting method.

In some embodiments, a detection technique may analyze only one formulation or cargo at a time. In some embodiments, a detection technique may analyze multiple formulations or cargos at a time. In some embodiments, a detection technique may analyze about 1 formulation, 2 formulations, 3 formulations, 4 formulations 5, formulations, 6 formulations, 7, formulations, 8, formulations, 9 formulations, 10 formulations, 11 formulations, 12 formulations, 13 formulations, 14 formulations, 15 formulations, 16 formulations, 17 formulations, 18 formulations, 19 formulations, 20 formulations, 21 formulations, 22 formulations, 23 formulations, 24 formulations, 25 formulations, or more at a time. In some embodiments, a detection technique may analyze between about 1 and 100 formulations. As a non-limiting example, a detection technique may analyze about 1-10, 1-20, 1-30, 1-40. 1-50, 1-60, 1-70. 1-80, or 1-90 formulations. In some embodiments, a detection technique may analyze more than 100 formulations at a time.

In some embodiments, a library of targeting systems may be analyzed. As a non-limiting examples, targeting systems may have the same formulation and different identifier sequences or moieties. As another non-limiting example, targeting systems may have the same formulation and the same identifier sequences or moieties. As another non-limiting example, targeting systems may have different formulations and the same identifier sequence or moieties. As another non-limiting example, targeting systems may have different formulations and different identifier sequences of moieties.

In some embodiments, a library of targeting systems may have one identifier sequence or moiety for analysis.

In some embodiments, a library of targeting systems may have at least two identifier sequences or moieties for analysis. The library may have 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more unique identifiers, e.g., from 2 to 50, from 2 to 100, from 2 to 100 unique identifiers, or from 2 to 5000 unique identifiers. identifier sequences or moieties for analysis.

In some embodiments, a library of targeting systems may have at least one originator constructs or benchmark constructs formulated in a nanoparticle delivery vehicle. The library may have 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1,000, 1,025, 1,050, 1,075, 1,100, 1,125, 1,150, 1,175, 1,200, 1,225, 1,250, 1,275, 1,300, 1,325, 1,350, 1,375, 1,400, 1,425, 1,450, 1,475, 1,500, 1,525, 1,550, 1,575, 1,600, 1,625, 1,650, 1,675, 1,700, 1,725, 1,750, 1,775, 1,800, 1,825, 1,850, 1,875, 1,900, 1,925, 1,950, 1,975, 2,000, 2,025, 2,050, 2,075, 2,100, 2,125, 2,150, 2,175, 2,200, 2,225, 2,250, 2,275, 2,300, 2,325, 2,350, 2,375, 2,400, 2,425, 2,450, 2,475, 2,500, 2,525, 2,550, 2,575, 2,600, 2,625, 2,650, 2,675, 2,700, 2,725, 2,750, 2,775, 2,800, 2,825, 2,850, 2,875, 2,900, 2,925, 2,950, 2,975, 3,000, 3,025, 3,050, 3,075, 3,100, 3,125, 3,150, 3,175, 3,200, 3,225, 3,250, 3,275, 3,300, 3,325, 3,350, 3,375, 3,400, 3,425, 3,450, 3,475, 3,500, 3,525, 3,550, 3,575, 3,600, 3,625, 3,650, 3,675, 3,700, 3,725, 3,750, 3,775, 3,800, 3,825, 3,850, 3,875, 3,900, 3,925, 3,950, 3,975, 4,000, 4,025, 4,050, 4,075, 4,100, 4,125, 4,150, 4,175, 4,200, 4,225, 4,250, 4,275, 4,300, 4,325, 4,350, 4,375, 4,400, 4,425, 4,450, 4,475, 4,500, 4,525, 4,550, 4,575, 4,600, 4,625, 4,650, 4,675, 4,700, 4,725, 4,750, 4,775, 4,800, 4,825, 4,850, 4,875, 4,900, 4,925, 4,950, 4,975, 5,000, 5,025, 5,050, 5,075, 5,100, 5,125, 5,150, 5,175, 5,200, 5,225, 5,250, 5,275, 5,300, 5,325, 5,350, 5,375, 5,400, 5,425, 5,450, 5,475, 5,500, 5,525, 5,550, 5,575, 5,600, 5,625, 5,650, 5,675, 5,700, 5,725, 5,750, 5,775, 5,800, 5,825, 5,850, 5,875, 5,900, 5,925, 5,950, 5,975, 6,000, 6,025, 6,050, 6,075, 6,100, 6,125, 6,150, 6,175, 6,200, 6,225, 6,250, 6,275, 6,300, 6,325, 6,350, 6,375, 6,400, 6,425, 6,450, 6,475, 6,500, 6,525, 6,550, 6,575, 6,600, 6,625, 6,650, 6,675, 6,700, 6,725, 6,750, 6,775, 6,800, 6,825, 6,850, 6,875, 6,900, 6,925, 6,950, 6,975, 7,000, 7,025, 7,050, 7,075, 7,100, 7,125, 7,150, 7,175, 7,200, 7,225, 7,250, 7,275, 7,300, 7,325, 7,350, 7,375, 7,400, 7,425, 7,450, 7,475, 7,500, 7,525, 7,550, 7,575, 7,600, 7,625, 7,650, 7,675, 7,700, 7,725, 7,750, 7,775, 7,800, 7,825, 7,850, 7,875, 7,900, 7,925, 7,950, 7,975, 8,000, 8,025, 8,050, 8,075, 8,100, 8,125, 8,150, 8,175, 8,200, 8,225, 8,250, 8,275, 8,300, 8,325, 8,350, 8,375, 8,400, 8,425, 8,450, 8,475, 8,500, 8,525, 8,550, 8,575, 8,600, 8,625, 8,650, 8,675, 8,700, 8,725, 8,750, 8,775, 8,800, 8,825, 8,850, 8,875, 8,900, 8,925, 8,950, 8,975, 9,000, 9,025, 9,050, 9,075, 9,100, 9,125, 9,150, 9,175, 9,200, 9,225, 9,250, 9,275, 9,300, 9,325, 9,350, 9,375, 9,400, 9,425, 9,450, 9,475, 9,500, 9,525, 9,550, 9,575, 9,600, 9,625, 9,650, 9,675, 9,700, 9,725, 9,750, 9,775, 9,800, 9,825, 9,850, 9,875, 9,900, 9,925, 9,950, 9,975, or 10,000 nanoparticles. As a non-limiting example, the nanoparticle may be a lipid nanoparticle.

### VII. Methods of Use

In some embodiments, the tropism delivery systems described herein may be used as a therapeutic to diagnose, prevent, treat and/or manage disease, disorders and conditions, or as a diagnostic. The therapeutic may be used in personalized medicine, immuno-oncology, cancer, vaccines, gene editing (e.g., CRISPR).

In some embodiments, the tropism delivery systems described herein may be used for diagnostic purposes or as diagnostic tools.

a. Infectious Disease

In some embodiments, the tropism delivery systems described herein may be used to diagnose, prevent, treat and/or manage infectious diseases. As used herein, the term “infectious disease” may refer to any disorder and/or condition caused by invasion into the body of an exogenous organism or infection agent that is not typically present such as, but not limited to, viruses, bacteria, prions, nematodes, fungus, parasites or arthropods. Infectious diseases are also known as transmissible diseases or communicable diseases. Infectious diseases and/or infection related diseases, disorders, and/or conditions that may be treated by methods, components and compositions of the present disclosure include, but are not limited to, Acute bacterial rhinosinusitis, 14-day measles, Acne, Acrodermatitis chronica atrophicans (ACA)-(late skin manifestation of latent Lyme disease), Acute hemorrhagic conjunctivitis, Acute hemorrhagic cystitis, Acute rhinosinusitis, Adult T-cell Leukemia-Lymphoma (ATLL), African Sleeping Sickness, AIDS (Acquired Immunodeficiency Syndrome), Alveolar hydatid, Amebiasis, Amebic meningoencephalitis, Anaplasmosis, Anthrax, Arboviral or parainfectious, Ascariasis-(Roundworm infections), Aseptic meningitis, Athlete's foot (Tinea pedis), Australian tick typhus, Avian Influenza, Babesiosis, Bacillary angiomatosis, Bacterial meningitis, Bacterial vaginosis, Balanitis, Balantidiasis, Bang's disease, Barmah Forest virus infection, Bartonellosis (Verruga peruana; Carrion's disease; Oroya fever), Bat Lyssavirus Infection, Bay sore (Chiclero's ulcer), Baylisascaris infection (Racoon roundworm infection), Beaver fever, Beef tapeworm, Bejel (endemic syphilis), Biphasic meningoencephalitis, Black Bane, Black death, Black piedra, Blackwater Fever, Blastomycosis, Blennorrhea of the newborn, Blepharitis, Boils, Bornholm disease (pleurodynia), Borrelia miyamotoi Disease, Botulism, Boutonneuse fever, Brazilian purpuric fever, Break Bone fever, Brill, Bronchiolitis, Bronchitis, Brucellosis (Bang's disease), Bubonic plague, Bullous impetigo, Burkholderia mallei (Glanders), Burkholderia pseudomallei (Melioidosis), Buruli ulcers (also Mycoburuli ulcers), Busse, Busse-Buschke disease (Cryptococcosis), California group encephalitis, Campylobacteriosis, Candidiasis, Canefield fever (Canicola fever; 7-day fever; Weil's disease; leptospirosis; canefield fever), Canicola fever, Capillariasis, Carate, Carbapenem-resistant Enterobacteriaceae (CRE), Carbuncle, Carrion's disease, Cat Scratch fever, Cave disease, Central Asian hemorrhagic fever, Central European tick, Cervical cancer, Chagas disease, Chancroid (Soft chancre), Chicago disease, Chickenpox (Varicella), Chiclero's ulcer, Chikungunya fever, Chlamydial infection, Cholera, Chromoblastomycosis, Ciguatera, Clap, Clonorchiasis (Liver fluke infection), Clostridium Difficile Infection, ClostriDium Perfringens (Epsilon Toxin), Coccidioidomycosis fungal infection (Valley fever; desert rheumatism), Coenurosis, Colorado tick fever, Condyloma accuminata, Condyloma accuminata(Warts), Condyloma lata, Congo fever, Congo hemorrhagic fever virus, Conjunctivitis, cowpox, Crabs, Crimean, Croup, Cryptococcosis, Cryptosporidiosis (Crypto), Cutaneous Larval Migrans, Cyclosporiasis, Cystic hydatid, Cysticercosis, Cystitis, Czechoslovak tick, D68 (EV-D68), Dacryocytitis, Dandy fever, Darling's Disease, Deer fly fever, Dengue fever (1, 2, 3 and 4), Desert rheumatism, Devil's grip, Diphasic milk fever, Diphtheria, Disseminated Intravascular Coagulation, Dog tapeworm, Donovanosis, Donovanosis (Granuloma inguinale), Dracontiasis, Dracunculosis, Duke's disease, Dum Dum Disease, Durand-Nicholas-Favre disease, Dwarf tapeworm, E. coli infection (E. coli), Eastern equine encephalitis, Ebola Hemorrhagic Fever (Ebola virus disease EVD), Ectothrix, Ehrlichiosis (Sennetsu fever), Encephalitis, Endemic Relapsing fever, Endemic syphilis, Endophthalmitis, Endothrix, Enterobiasis (Pinworm infection), Enterotoxin-B Poisoning (Staph Food Poisoning), Enterovirus Infection, Epidemic Keratoconjunctivitis, Epidemic Relapsing fever, Epidemic typhus, Epiglottitis, Erysipelis, Erysipeloid (Erysipelothricosis), Erythema chronicum migrans, Erythema infectiosum, Erythema marginatum, Erythema multiforme, Erythema nodosum, Erythema nodosum leprosum, Erythrasma, Espundia, Eumycotic mycetoma, European blastomycosis, Exanthem subitum (Sixth disease), Eyeworm, Far Eastern tick, Fascioliasis, Fievre boutonneuse(Tick typhus), Fifth Disease (erythema infectiosum), Filatow-Dukes' Disease (Scalded Skin Syndrome; Ritter's Disease), Fish tapeworm, Fitz-Hugh-Curtis syndrome-Perihepatitis, Flinders Island Spotted Fever, Flu (Influenza), Folliculitis, Four Corners Disease, Four Corners Disease (Human Pulmonary Syndrome (HPS)), Frambesia, Francis disease, Furunculosis, Gas gangrene, Gastroenteritis, Genital Herpes, Genital Warts, German measles, Gerstmann-Straussler-Scheinker (GSS), Giardiasis, Gilchrist's disease, Gingivitis, Gingivostomatitis, Glanders, Glandular fever (infectious mononucleosis), Gnathostomiasis, Gonococcal Infection (Gonorrhea), Gonorrhea, Granuloma inguinale (Donovanosis), Guinea Worm, Haemophilus Influenza disease, Hamburger disease, Hansen's disease—leprosy, Hantaan disease, Hantaan-Korean hemorrhagic fever, Hantavirus Pulmonary Syndrome, Hantavirus Pulmonary Syndrome (HPS), Hard chancre, Hard measles, Haverhill fever-Rat bite fever, Head and Body Lice, Heartland fever, Helicobacterosis, Hemolytic Uremic Syndrome (HUS), Hepatitis A, Hepatitis B, Hepatitis C, Hepatitis D, Hepatitis E, Herpangina, Herpes-genital, Herpes labialis, Herpes-neonatal, Hidradenitis, Histoplasmosis, Histoplasmosis infection (Histoplasmosis), His-Werner disease, HIV infection, Hookworm infections, Hordeola, Hordeola (Stye), HTLV, HTLV-associated myelopathy (HAM), Human granulocytic ehrlichiosis, Human monocytic ehrlichiosis, Human Papillomavirus (HPV), Human Pulmonary Syndrome, Hydatid cyst, Hydrophobia, Impetigo, Including congenital (German Measles), Inclusion conjunctivitis, Inclusion conjunctivitis-Swimming Pool conjunctivitis-Pannus, Infantile diarrhea, Infectious Mononucleosis, Infectious myocarditis, Infectious pericarditis, Influenza, Isosporiasis, Israeli spotted fever, Japanese Encephalitis, Jock itch, Jorge Lobo disease-lobomycosis, Jungle yellow fever, Junin Argentinian hemorrhagic fever, Kala Azar, Kaposi's sarcoma, Keloidal blastomycosis, Keratoconjunctivitis, Kuru, Kyasanur forest disease, LaCrosse encephalitis, Lassa hemorrhagic fever, Legionellosis (Legionnaires Disease), Legionnaire's pneumonia, Lemierre's Syndrome (Postanginal septicemia), Lemming fever, Leprosy, Leptospirosis (Nanukayami fever; Weil's disease), Listeriosis (Listeria), Liver fluke infection, Lobo's mycosis, Lockjaw, Loiasis, Louping Ill, Ludwig's angina, Lung fluke infection, Lung fluke infection (Paragonimiasis), Lyme disease, Lymphogranuloma venereum infection (LGV), Machupo Bolivian hemorrhagic fever, Madura foot, Mal del pinto, Malaria, Malignant pustule, Malta fever, Marburg hemorrhagic fever, Masters disease, Maternal Sepsis (Puerperal fever), Measles, Mediterranean spotted fever, Melioidosis (Whitmore's disease), Meningitis, Meningococcal Disease, MERS, Milker's nodule, Molluscum contagiosum, Moniliasis, monkeypox, Mononucleosis, Mononucleosis-like syndrome, Montezuma's Revenge, Morbilli, MRSA (methicillin-resistant Staphylococcus aureus) infection, Mucormycosis-Zygomycosis, Multiple Organ Dysfunction Syndrome or MODS, Multiple-system atrophy (MSA), Mumps, Murine typhus, Murray Valley Encephalitis(MVE), Mycoburuli ulcers, Mycoburuli ulcers-Buruli ulcers, Mycotic vulvovaginitis, Myositis, Nanukayami fever, Necrotizing fasciitis, Necrotizing fasciitis-Type 1, Necrotizing fasciitis-Type 2, Negishi, New world spotted fever, Nocardiosis, Nongonococcal urethritis, Non-Polio (Non-Polio Enterovirus), Norovirus infection, North American blastomycosis, North Asian tick typhus, Norwalk virus infection, Norwegian itch, O'Hara disease, Omsk hemorrhagic fever, Onchoceriasis, Onychomycosis, Opisthorchiasis, Opthalmia neonatorium, Oral hairy leukoplakia, Orf, Oriental Sore, Oriental Spotted Fever, Ornithosis (Parrot fever; Psittacosis), Oroya fever, Otitis externa, Otitis media, Pannus, Paracoccidioidomycosis, Paragonimiasis, Paralytic Shellfish Poisoning (Paralytic Shellfish Poisoning), Paronychia (Whitlow), Parotitis, PCP pneumonia, Pediculosis, Peliosis hepatica, Pelvic Inflammatory Disease, Pertussis (also called Whooping cough), Phaeohyphomycosis, Pharyngoconjunctival fever, Piedra (White Piedra), Piedra(Black Piedra), Pigbel, Pink eye conjunctivitis, Pinta, Pinworm infection, Pitted Keratolysis, Pityriasis versicolor (Tinea versicolor), Plague; Bubonic, Pleurodynia, Pneumococcal Disease, Pneumocystosis, Pneumonia, Pneumonic (Plague), Polio or Poliomyelitis, Polycystic hydatid, Pontiac fever, Pork tapeworm, Posada-Wernicke disease, Postanginal septicemia, Powassan, Progressive multifocal leukencephalopathy, Progressive Rubella Panencephalitis, Prostatitis, Pseudomembranous colitis, Psittacosis, Puerperal fever, Pustular Rash diseases (Small pox), Pyelonephritis, Pylephlebitis, Q-Fever, Quinsy, Quintana fever (5-day fever), Rabbit fever, Rabies, Racoon roundworm infection, Rat bite fever, Rat tapeworm, Reiter Syndrome, Relapsing fever, Respiratory syncytial virus (RSV) infection, Rheumatic fever, Rhodotorulosis, Ricin Poisoning, Rickettsialpox, Rickettsiosis, Rift Valley Fever, Ringworm, Ritter's Disease, River Blindness, Rocky Mountain spotted fever, Rose Handler's disease (Sporotrichosis), Rose rash of infants, Roseola, Ross River fever, Rotavirus infection, Roundworm infections, Rubella, Rubeola, Russian spring, Salmonellosis gastroenteritis, San Joaquin Valley fever, Sao Paulo Encephalitis, Sao Paulo fever, SARS, Scabies Infestation (Scabies) (Norwegian itch), Scalded Skin Syndrome, Scarlet fever (Scarlatina), Schistosomiasis, Scombroid, Scrub typhus, Sennetsu fever, Sepsis (Septic shock), Severe Acute Respiratory Syndrome, Severe Acute Respiratory Syndrome (SARS), Shiga Toxigenic Escherichia coli (STEC/VTEC), Shigellosis gastroenteritis (Shigella), Shinbone fever, Shingles, Shipping fever, Siberian tick typhus, Sinusitis, Sixth disease, Slapped cheek disease, Sleeping sickness, Smallpox (Variola), Snail Fever, Soft chancre, Southern tick associated rash illness, Sparganosis, Spelunker's disease, Sporadic typhus, Sporotrichosis, Spotted fever, Spring, St. Louis encephalitis, Staphylococcal Food Poisoning, Staphylococcal Infection, Strep. throat, Streptococcal Disease, Streptococcal Toxic-Shock Syndrome, Strongyloiciasis, Stye, Subacute Sclerosing Panencephalitis, Subacute Sclerosing Panencephalitis (SSPE), Sudden Acute Respiratory Syndrome, Sudden Rash, Swimmer's ear, Swimmer's Itch, Swimming Pool conjunctivitis, Sylvatic yellow fever, Syphilis, Systemic Inflammatory Response Syndrome (SIRS), Tabes dorsalis (tertiary syphilis), Taeniasis, Taiga encephalitis, Tanner's disease, Tapeworm infections, Temporal lobe encephalitis, Temporal lobe encephalitis, tetani (Lock Jaw), Tetanus Infection, Threadworm infections, Thrush, Tick, Tick typhus, Tinea barbae, Tinea capitis, Tinea corporis, Tinea cruris, Tinea manuum, Tinea nigra, Tinea pedis, Tinea unguium, Tinea versicolor, Torulopsosis, Torulosis, Toxic Shock Syndrome, Toxoplasmosis, transmissible spongioform (CJD), Traveler's diarrhea, Trench fever 5, Trichinellosis, Trichomoniasis, Trichomycosis axillaris, Trichuriasis, Tropical Spastic Paraparesis (TSP), Trypanosomiasis, Tuberculosis (TB), Tuberculousis, Tularemia, Typhoid Fever, Typhus fever, Ulcus molle, Undulant fever, Urban yellow fever, Urethritis, Vaginitis, Vaginosis, Vancomycin Intermediate (VISA), Vancomycin Resistant (VRSA), Varicella, Venezuelan Equine encephalitis, Verruga peruana, Vibrio cholerae (Cholera), Vibriosis (Vibrio), Vincent's disease or Trench mouth, Viral conjunctivitis, Viral Meningitis, Viral meningoencephalitis, Viral rash, Visceral Larval Migrans, Vomito negro, Vulvovaginitis, Warts, Waterhouse, Weil's disease, West Nile Fever, Western equine encephalitis, Whipple's disease, Whipworm infection, White Piedra, Whitlow, Whitmore's disease, Winter diarrhea, Wolhynia fever, Wool sorters' disease, Yaws, Yellow Fever, Yersinosis, Yersinosis (Yersinia), Zahorsky's disease, Zika virus disease, Zoster, Zygomycosis, John Cunningham Virus (JCV), Human immunodeficiency virus (HIV), Influenza virus, Hepatitis B, Hepatitis C, Hepatitis D, Respiratory syncytial virus (RSV), Herpes simplex virus 1 and 2, Human Cytomegalovirus, Epstein-Barr virus, Varicella zoster virus, Coronaviruses, Poxviruses, Enterovirus 71, Rubella virus, Human papilloma virus, Streptococcus pneumoniae, Streptococcus viridans., Staphylococcus aureus (S. aureus), Methicillin-resistant Staphylococcus aureus (MRSA), Vancomycin-intermediate Staphylococcus aureus (VISA), Vancomycin-resistant Staphylococcus aureus (VRSA), Staphylococcus epidermidis (S. epidermidis), Clostridium Tetani, Bordetella pertussis, Bordetella paratussis, Mycobacterium, Francisella Tularensis, Toxoplasma gondii, Candida (C. albicans, C. glabrata, C. parapsilosis, C. tropicalis, C. krusei and C. lusitaniae) and/or any other infectious diseases, disorders or syndromes.

Additionally, an infection or symptoms associated with an infection may be caused by one or more toxins produced by such agents. Humans, and other mammals, react to infections with an innate immune system response, often involving an inflammation. The illnesses and symptoms involved with infections vary according to the infectious agent. Many infections may be subclinical without presenting any definite or observable symptoms, whereas some infections cause severe symptoms, require hospitalization or may be life-threatening. Some infections are localized, whereas some may overcome the body through blood circulation or lymphatic vessels. Some infections have long-term effects on wellbeing of infected individuals.

Infectious agents may be transmitted to humans via different routes. For example, infection agents may be transmitted by direct contact with an infected human, an infected animal, or an infected surface. Infections may be transmitted by direct contact with bodily fluids of an infected human or an animal, e.g. blood, saliva, sweat, tears, mucus, female ejaculate, semen, vomit or urine. For example, infection may be transmitted by a fecal-oral route, referring to an infected person shedding the virus in fecal particles which then enters to person's mouth causing infection. The fecal-oral route is especially common transmission route in environments with poor sanitation and hygiene. Non-limiting examples of agents transmitted by the fecal-oral route include bacteria, e.g. shigella, Salmonella typhi and Vibrio cholerae, virus, e.g. norovirus, rotavirus, enteroviruses, and hepatitis A, fungi, protozoans e.g. Entamoeba histolytica, parasites, tape worms, transmitted by contaminated food or beverage, leading to food poisoning or gastroenteritis. Infections may be transmitted by a respiratory route, referring to agents that are spread through the air. Typical examples include agents spread as small droplets of liquid or as aerosols, e.g. respiratory droplets expelled from the mouth and nose while coughing and sneezing. Typical examples of respiratory transmitted diseases include the common cold mostly implicated to rhinoviruses, influenza caused by influenza viruses, respiratory tract infections caused by e.g. respiratory syncytial virus (RSV). Infections may be transmitted by a sexual transmission route. Examples of common sexually transmitted infections include e.g. human immunodeficiency virus (HIV) causing acquired immune deficiency syndrome (AIDS), chlamydia caused by Neisseria gonorrhoeae bacteria, fungal infection Candidiasis caused by Candida yeast, and Herpes Simplex disease caused by herpes simplex virus. Infections may be transmitted by an oral transmission route, e.g. by kissing or sharing a drinking glass. A common infection transmitted by oral transmission is an infectious mononucleosis caused by Epstein-Barr virus. Infections may be transmitted by a vertical transmission, also known as “mother-to-child transmission,” from mother to an embryo, fetus or infant during pregnancy or childbirth. Examples of infection agents that may be transmitted vertically include HIV, chlamydia, rubella, Toxoplasma gondii, and herpes simplex virus. Infections may be transmitted by an iatrogenic route, referring to a transmission by medical procedures such as injection (contaminated reused needles and syringes), or transplantation of infected material, blood transfusions, or infection occurring during surgery. For example, methicillin-resistant Staphylococcus aureus (MRSA), which may cause several severe infections, may be transmitted via iatrogenic route during surgery. Infections may also be transmitted by vector-borne transmission, where a vector may be an organism transferring the infection agents from one host to another. Such vectors may be triatomine bugs, e.g. trypanosomes, parasites, animals, arthropods including e.g. mosquitos, flies, lice, flees, tick and mites or humans. Non-limiting examples of mosquito-borne infections include Dengue fever, West Nile virus related infections, Yellow fever and Chikungunya fever. Non-limiting examples of parasite-borne diseases include malaria, Human African trypanosomiasis and Lyme disease. Non-limiting examples of diseases spread by humans or mammals include HIV, Ebola hemorrhagic fever and Marburg fever.

b. Non-Infectious Disease

The tropism delivery systems described herein may be used to diagnose, prevent, treat and/or manage non-infectious diseases. Non-limiting examples of non-infectious diseases include immune system diseases, autoimmune diseases, inflammatory diseases, stroke, migraine, pain, neuropathies, psychiatric disorders including schizophrenia, bipolar disorder, and autism, cancer, ocular diseases, muscle diseases, endocrine and metabolic diseases, nervous system diseases, and systemic diseases of the blood, heart and bone.

In some embodiments, the non-infections disease is a muscle disease, including but not limited to polymyositis, dermatomyositis, muscular dystrophy, myasthenia gravis, amyotrophic lateral sclerosis, rhabdomyolysis, cardiomyopathy, and sarcopenia.

In some embodiments, the non-infectious disease is a central nervous system (CNS) disease, including but not limited to disorders caused by trauma, infections, degeneration, structural defects, tumors, autoimmune disorders, vascular disorders, or stroke. The CNS disorders include but are not limited to parkinson's disease, multiple sclerosis, amyotrophic lateral sclerosis, alzheimer's disease, huntington's disease, peripheral neuropathies, encephalitis or abscesses of the brain, and meningitis.

c. Cancer and Immuno-Oncology

In some embodiments, the tropism delivery systems described herein may be used to diagnose, prevent, treat and/or manage immuno-oncology (I-0) disease, disorders and/or conditions.

In some embodiments, the tropism delivery systems described herein may be used to develop an immunotherapy or as an immunotherapy in an immuno-oncology treatment of a subject suffering from cancer. Non-limiting examples of immuno-oncology applications include active, passive or hybrid immunotherapies, checkpoint blockade, adoptive cell transfer (ACT), cancer vaccines, CAR or CAR-T therapies, dendritic cell therapy, stem cell therapies, natural killer (NK) cell-based therapies, and interferon or interleukin based methods.

In some embodiments, the tropism delivery systems described herein may be used to diagnose, prevent, treat and/or manage cancer.

Cancer is a group of more than 100 diseases associated with abnormal division and cell growth with characteristic spreading in the body. Many cancers are in the form of tumors, e.g. breast cancer, lung cancer, colon cancer, ovarian cancer, renal cancer, prostate cancer, head and neck cancer, pancreas cancer, bone cancer, and thyroid cancer. Cancers associated with blood and lymphoid tissues may be referred to as liquid tumors, e.g. leukemia, lymphoma and myeloma. Cancer is caused by failure of tissue growth regulation. Genes associated with cancer include oncogenes, that promote cell growth and reproduction, and tumor suppressor genes, that inhibit cell division. Oncogenes include, but are not limited to, growth factors, receptor and cytoplasmic tyrosine kinases, transcription factors, serine/threonine kinases and regulatory GTPases. Tumor protein p53 is the most common tumor suppressor protein found in more than half of cancer types. Susceptibility to cancer is involved with environmental factors, as well as genetic. Though progress with prevention, diagnosis and treatment of cancer has been tremendous, cancer remains a severe and life-threatening disease. According to American Cancer Society, an estimated 1.6 cancers are diagnosed annually in the US, leading to more than a half a million deaths.

In one embodiment, the cancer may be Leptomeningial metastases and/or glioblastoma.

Therapies associated with cancer treatment include surgery, chemotherapy, radiation and antibody therapies. Antibodies for treatment and/or prevention of cancers have been on the market for nearly two decades, and are considered one of the most important strategies for treatment of e.g. hematological malignancies and solid tumors. A number of cancer-associated antigens have been identified for treatment of cancers. Antibodies targeting such antigens may be used to diagnose, prevent and/or treat the associated cancers (see, e.g. Scott et al, 2012, Nature Reviews Cancer 12, 278-287, and references therein).

Some solid cancer tumors are associated with expressed glycoproteins antigens. Such antigens include, but are not limited to, EPCAM (Epithelial cell adhesion molecule), CEA (Carcinoembryonic antigen), gpA33 (Glycoprotein A33 (Transmembrane)), mucins, TAG-72 (Tumor-associated glycoprotein 72), CAIX (Carbonic anhydrase IX), PSMA (Prostate-specific membrane antigen), and FBP (Folate-binding protein). Antibodies targeting the expressed glycoproteins may be used to treat associated tumors. Such solid tumors include, but are not limited to, breast, colon cancer, lung, colorectal, ovarian, renal cell, and/or prostate tumors.

Some solid cancer tumors are associated with growth factor and differentiation signaling associated antigens. Such antigens include, but are not limited to, EGFR/ERBB1/HER1 (epidermal growth factor receptor 1), ERBB2 (epidermal growth factor receptor 2), ERBB3 (epidermal growth factor receptor 3), MET (Tyrosine-Protein Kinase Met), IGF1R (insulin-like growth factor 1 receptor), EPHA3 (EPH Receptor A3), TRAILR1, (Death receptor 4), and (Receptor activator of nuclear factor kappa-B ligand). Cancers that may be treated with antibodies targeting the growth factor and differentiation signaling include, but are not limited to, breast, colon, lung, ovarian, prostate, head and neck, pancreas, thyroid, kidney, and colon tumors, melanoma, glioma, bone metastases, and hematological malignancies.

Some cancer tumors are associated with antigens of stromal and extracellular matrix. Such antigens include, but are not limited to, tenascin and FAP (Fibroblast Activation Protein, Alpha). Cancers that may be treated with antibodies targeting the stromal and extracellular matrix antibodies include, but are not limited to, breast, prostate, colon, lung, pancreas and head and neck tumors and glioma.

Some cancer tumors are associated with such as Lewis —Y Le(y) antigen. Le(y) antigen has been found expressed on a number of cancers, such as, but not limited to, ovarian, breast, colon, lung and prostate cancer. Antibodies targeting Le(y) antigen may be used to treat the associated cancers.

Some cancer tumors are associated with glycolipid antigens. Such antigens include, but are not limited to, gangliosides, such as GD2, GD3, and GM2 (monosialotetrahexosylganglioside 2). Cancers that may be treated with antibodies targeting the glycolipid antigens include, but are not limited to, epithelial tumors (e.g. breast, colon and lung tumors) and neuroectodermal tumors (tumors of the central and peripheral nervous system).

The vasculature of solid tumors is abnormal, compared to normal vasculature. Antigens supporting the formation of abnormal microvasculature and progress of cancer include, but are not limited to, VEGF (Vascular endothelial growth factor), VEGFR (vascular endothelial growth factor receptor), integrin αVβ and integrin α5β1. Antibodies targeting such antigens may be used to treat a number of solid tumors such as, but not limited to, lung, breast, renal, brain, eye, colorectal, melanoma, ovarian, and/or other tumors, by preventing the formation of abnormal vasculature.

Hematopoietic and lymphoid malignancies are cancers affecting the blood, bone marrow, lymph and lymphatic system. Such cancers include e.g. leukemias (acute and chronic lymphoblastic leukemia, acute and chronic myelogenous leukemia), lymphomas (Hodgkin's lymphoma, Non-Hodgkin's lymphoma) and myelomas. Tumors of the hematopoietic and lymphoid tissues are closely related to immune systems. Hematological tumors may be caused by chromosomal abnormalities derived from the myeloid and lymphoid cell lines. The lymphoid cell line produces T and B cells, whereas myeloid cell line produces granulocytes, erythrocytes, thrombocytes, macrophages and mast cells. T and B cell associated hematopoietic differentiation antigens are glycoproteins that are usually from cluster of differentiation (CD) group, such as, but not limited to, CD20, CD30, CD33 and CD52. Antibodies targeting such antigens may be used for prevention and/or treatment of hematopoietic and lymphoid cancers.

d. Immune and Autoimmune Diseases

In some embodiments, the tropism delivery systems described herein may be used to diagnose, prevent, treat and/or manage immune diseases.

In some embodiments, the tropism delivery systems described herein may be used to diagnose, prevent, treat and/or manage autoimmune diseases. In some embodiments, the tropism delivery systems described herein may be used to treat subjects suffering from an autoimmune disease. In some embodiments, the tropism delivery systems described herein may be used to treat subjects suspected of developing an autoimmune disease.

In some embodiments, the tropism delivery systems described herein may be used to treat autoimmune diseases such as systemic sclerosis. In one embodiment, the payload region of the originator construct and/or the benchmark construct may encode antibodies or fragments thereof that target anti-neutrophil cytoplasmic antibodies (ANCA). In some embodiments, the tropism delivery systems described herein may be used to treat ANCA-associated vasculitis.

The human immune system is a complex mechanism for identifying and removing harmful environmental agents and repairing the harm and damage caused by them. In general, immune system identifies the body's own substances from substances acquired, in other words, the self from the non-self. The immune system can be subdivided into innate and adaptive systems. The innate system is always present and includes macrophages, dendritic cells, myeloid cells (neutrophils, mast cells, basophils, eosinophils) NK cells, complement factors and cytokines. The adaptive system responds to infectious agents, and includes T and B lymphocytes, antibodies and cytokines. Activation of T and B cells in the absence of an infectious agents leads to autoimmune diseases (see, e.g. Mackay et al., 2001, N Engl J Med, Vol. 345, No. 5, and references therein). Autoimmune diseases may affect several tissues and biological functions, e.g. joints, skin, blood vessels, muscles, organs, intestine etc. Autoimmune diseases arise from an overactive and misguided immune response to the body's natural tissues and species. Autoimmune diseases and conditions include, but are not limited to, rheumatoid arthritis, diabetes type 1, systemic lupus erythematosus, celiac sprue, psoriasis, Graves' disease, and Lyme disease. Autoimmune diseases may be caused by infections, drugs, environmental irritants, toxins, and/or genetic factors. Autoimmune diseases affect up to 50 million individuals in the US. Two most common autoimmune diseases are rheumatoid arthritis and autoimmune thyroiditis, together affecting approximately 5% of population in Western countries.

Though medical therapies for autoimmune diseases exits, the diseases may still significantly lower the quality of life, or even be fatal. There remains a need for medical therapies affecting the pathophysiology of autoimmune diseases. Autoimmune disease pathophysiology is associated with several factors and may be prevented and/or treated by antibodies targeting associated proteins. Such targets include, but are not limited to, infectious agents, environmental triggers (e.g. gliadin), targets affecting cytokine production or signaling (e.g. TNFa (tumor necrosis factor alpha),IL-1 (interleukin 1-receptor), IL-2 (interleukin-2), IL-2R (interleukin-2 receptor), IL-7 (interleukin-7), IL-10 (interleukin-10), IL-10R (interleukin-10 receptor), interferon-y, STAT-3 (Signal transducer and activator of transcription 3), STAT-4 (Signal transducer and activator of transcription 4), TGF beta (transforming growth factor beta), T cell trans TGF beta), T cell regulators (e.g. CTLA4 (Cytotoxic T-Lymphocyte-Associated Protein 4)), complement components (e.g. C₁ and C₄), TNFa (tumor necrosis factor alpha) and TNFb (tumor necrosis factor beta), T cell regulators (e.g. CD1), epitopes of B and T cells, and/or other targets, such as those associated with B and C cells.

e. Inflammatory Diseases

In some embodiments, the tropism delivery systems described herein may be used to diagnose, prevent, treat and/or manage inflammatory diseases.

In some embodiments, the tropism delivery systems described herein may be used to treat subjects suffering from an inflammatory disease.

In some cases, the tropism delivery systems described herein may be used to treat subjects suspected of developing an inflammatory disease.

Inflammation is a natural response of the body to an irritation e.g. by infection, damaged cells or other harmful agents. The purpose of the inflammation is to remove the cause of irritation and necrotic cells and damaged tissues and to initiate cell and tissue repair. Inflammation has a role in most diseases. Inflammatory disorders are abnormalities in the body's ability to regulate inflammation. Over 100 disorders associated with high levels of inflammation have been identified, including, but not limited to, Alzheimer's disease, ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease, and ulcerative colitis. Many inflammatory disorders are severe, and even life-threatening. Antibodies targeting proteins associated with inflammation may be used to prevent, manage or treat inflammatory disorders as well as inflammation associated diseases.

A large number of proteins are associated in inflammation, including, but not limited to, TNF (anti-tumor necrosis factor), IL-1R (Interleukin-1 receptor), IL-6R (Interleukin-6 receptor), Alpha integrin subunit, CTLA4 (Cytotoxic T-Lymphocyte-Associated Protein 4), and CD20 (see, e.g. Kotsovilis and Andreakos, 2014, Michael Steinitz (ed.), Human Monoclonal Antibodies: Methods and Protocols, Methods in Molecular Biology, vol. 1060, and references therein). For example, adalimumab (developed by Abbot Laboratories) is a TNF-targeting antibody for rheumatoid arthritis and other arthritis, psoriasis, and Crohn's disease and Natalizumab (developed by Biogen Idec) is an antibody targeting alpha 4-integrin for treatment of Crohn's disease. Additionally, plethora of cytokines, chemokines, adhesion and co-stimulatory molecules, receptors, as well as diverse cell types, may have a role in inflammatory diseases.

f. Preventative Applications

In some embodiments, the tropism delivery systems described herein may be used to prevent disease or stabilize the progression of disease.

In some embodiments, the tropism delivery systems described herein may be used as a prophylactic to prevent a disease or disorder in the future.

In some embodiments, the tropism delivery systems described herein may be used to halt further progression of a disease or disorder.

g. Vaccine

In some embodiments, the tropism delivery systems described herein may be used as, and/or in a manner similar to that of a vaccine. As used herein, a “vaccine” is a biological preparation that improves immunity to a particular disease or infectious agent.

In some embodiments, the tropism delivery systems described herein may be used as, and/or in a manner similar to that of a vaccine for a therapeutic area such as, but not limited to, cardiovascular, CNS, dermatology, endocrinology, oncology, immunology, respiratory, and anti-infective.

In some embodiments, the tropism delivery systems described herein may be used as a vaccine to diagnose, prevent, treat and/or manage influenza.

In some embodiments, the tropism delivery systems described herein may be used as a prophylactic to diagnose, prevent, treat and/or manage HIV.

In some embodiments, the tropism delivery systems described herein may be used as a prophylactic to diagnose, prevent, treat and/or manage COVID-19.

In some embodiments, the tropism delivery systems described herein may be used as a prophylactic to diagnose, prevent, treat and/or manage polio.

In some embodiments, the tropism delivery systems described herein may be used as a prophylactic to diagnose, prevent, treat and/or manage tetanus.

In some embodiments, the tropism delivery systems described herein may be used as a prophylactic to diagnose, prevent, treat and/or manage Hepatitis A.

In some embodiments, the tropism delivery systems described herein may be used as a prophylactic to diagnose, prevent, treat and/or manage Hepatitis B.

In some embodiments, the tropism delivery systems described herein may be used as a prophylactic to diagnose, prevent, treat and/or manage Hepatitis C.

In some embodiments, the tropism delivery systems described herein may be used as a prophylactic to diagnose, prevent, treat and/or manage Rubella.

In some embodiments, the tropism delivery systems described herein may be used as a prophylactic to diagnose, prevent, treat and/or manage Hib (Haemophilus influenzae type b).

In some embodiments, the tropism delivery systems described herein may be used as a prophylactic to diagnose, prevent, treat and/or manage Measles.

In some embodiments, the tropism delivery systems described herein may be used as a prophylactic to diagnose, prevent, treat and/or manage Pertussis (Whooping Cough).

In some embodiments, the tropism delivery systems described herein may be used as a prophylactic to diagnose, prevent, treat and/or manage Pneumococcal Disease.

In some embodiments, the tropism delivery systems described herein may be used as a prophylactic to diagnose, prevent, treat and/or manage Rotavirus.

In some embodiments, the tropism delivery systems described herein may be used as a prophylactic to diagnose, prevent, treat and/or manage Mumps.

In some embodiments, the tropism delivery systems described herein may be used as a prophylactic to diagnose, prevent, treat and/or manage Chickenpox.

In some embodiments, the tropism delivery systems described herein may be used as a prophylactic to diagnose, prevent, treat and/or manage Diphtheria.

g. Contraceptive

In some embodiments, the tropism delivery systems described herein may be used as, and/or in a manner similar to that of a contraceptive. As used herein, the term, “contraceptive” may be defined as any agent or method that may be used to prevent pregnancy.

In some embodiments, the contraceptive may be used short-term or long-term.

In some embodiments, the contraceptive may be reversible or permanent.

h. Diagnostics

In some embodiments, the tropism delivery systems described herein may be used for diagnostic purposes or as diagnostic tools for any of the aforementioned diseases or disorders.

In some embodiments, the tropism delivery systems described herein may be used to detect a biomarker for disease diagnosis.

In some embodiments, the tropism delivery systems described herein may be used for diagnostic imaging purposes, e.g., MRI, PET, CT or ultrasound.

h. Research

In some embodiments, the tropism delivery systems described herein may be used for diagnostic purposes or as research tools for any of the aforementioned diseases or disorders.

In some embodiments, the tropism delivery systems described herein may be used to detect a biomarker for research.

In some embodiments, the tropism delivery systems described herein may be used in any research experiment, e.g., in vivo or in vitro experiments.

In some embodiments, the tropism delivery systems described herein may be used in cultured cells. The cultured cells may be derived from any origin known to one with skill in the art, and may be as non-limiting examples, derived from a stable cell line, an animal model or a human patient or control subject.

In some embodiments, the tropism delivery systems described herein may be used in in vivo experiments in animal models (e.g., mouse, rat, rabbit, dog, cat, non-human primate, guinea pig, ferret, c-elegans, drosophila, zebrafish, or any other animal used for research purposes, known in the art).

In some embodiments, the tropism delivery systems described herein may be used in human research experiments or human clinical trials.

In some embodiments, the tropism delivery systems described herein may be used in stem cells and/or cell differentiation

### i. Additional Exemplary Embodiments

This disclosure also relates to the additional exemplary embodiments that are enumerated below:

This disclosure relates to a library of polynucleotides, wherein each of the polynucleotides in said library comprises:
a. a payload sequence region, said payload sequence region comprising a coding nucleic acid sequence or non-coding nucleic acid sequence;
b. at least one flanking sequence region located upstream or downstream of the payload sequence region;
c. at least one unique identifier; and
d. optionally, at least one regulatory sequence region.

Libraries of polynucleotides of this disclosure can comprise polynucleotides that are DNA.

This disclosure relates to libraries of polynucleotides, wherein the polynucleotides are RNA.

Libraries of polynucleotides of this disclosure can comprise RNA, wherein the RNA are circular RNA (oRNA).

Libraries of polynucleotide of this disclosure can comprise RNA, wherein the RNA are short interfering RNA (siRNA)

This disclosure relates to libraries of circular RNA (oRNA), wherein the oRNA inhibits or suppresses the expression of a target of interest in a cell. The inhibition or suppression by libraries of oRNA of this disclosure can be about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

This disclosure relates to libraries of siRNA, wherein the siRNA inhibits or suppresses the expression of a target of interest in a cell. The inhibition or suppression by libraries of siRNA of this disclosure can be about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

This disclosure relates to libraries of polynucleotides comprised of DNA or RNA, wherein the polynucleotides are substantially circular. Libraries of polynucleotides comprising substantially circular DNA or RNA can comprise an internal ribosome entry site (IRES) sequence that is operably linked to the payload sequence region of the polynucleotide. Libraries comprising circular polynucleotides (e.g., circular DNA, circular RNA) can comprise an IRES sequence derived from picornavirus complementary DNA, encephalomyocarditis virus (EMCV) complementary DNA, poliovirus complementary DNA, or an Antennapedia gene from Drosophila melanogaster.

Libraries of this disclosure comprising substantially circular polynucleotides (e.g., circular DNA, oRNA) can comprise a termination element on the polynucleotides, wherein the termination element comprises at least one stop codon.

Libraries of this disclosure comprising substantially circular polynucleotides (e.g., circular DNA, oRNA) can comprise polynucleotides comprising a regulatory element.

Libraries of this disclosure comprising substantially circular polynucleotides (e.g., circular DNA, oRNA) can further comprise at least one masking agent on the polynucleotides.

Libraries of this disclosure comprising substantially circular polynucleotides (e.g., circular DNA, oRNA) may be produced using in vitro transcription.

Libraries of this disclosure comprising substantially circular polynucleotides (e.g., circular DNA, oRNA) can comprise a payload sequence, wherein the payload sequence region comprises a non-coding nucleic acid sequence.

In another example, libraries of this disclosure comprising substantially circular polynucleotides (e.g., circular DNA, oRNA) comprise a payload sequence, wherein the payload sequence region comprises a coding nucleic acid sequence.

The coding nucleic acid sequence of libraries described herein may encode a protein of interest for treatment of muscular dystrophy (e.g., Dystrophin).

The coding nucleic acid sequence of libraries described herein may encode a protein of interest for treatment of cardiovascular disease (e.g., SERCA2a, GATA4, Tbx5, Mef2C, Hand2, Myocd).

The coding nucleic acid sequence of libraries described herein may encode a protein of interest for treatment of neurodegenerative disease (e.g., NGF, BDNF, GDNF, or NT-3).

The coding nucleic acid sequence of libraries described herein may encode a protein of interest for treatment of chronic pain (e.g., GlyRal).

The coding nucleic acid sequence of libraries described herein may encode a protein of interest for treatment of a glutamate decarboxylase disease or disorder (e.g., GAD65 or GAD67).

The coding nucleic acid sequence of libraries described herein may encode a protein of interest for treatment of lung disease (e.g., CFTR).

The coding nucleic acid sequence of libraries described herein may encode a protein of interest for treatment of hemophilia (e.g., Factor VIII, Factor IX).

The coding nucleic acid sequence of libraries described herein may encode a protein of interest for treatment of neoplasia (e.g., PTEN, ATM, ATR, EGFR, ERBB2, ERBB3, ERBB4, Notch1, Notch2, Notch3, Notch4, AKT, AKT2, AKT3, HIF, HI Fla, HIF3a, Met, HRG, Bcl2, PPARalpha, PPAR gamma, WT1 (Wilms Tumor), FGF Receptor Family member 1, FGF Receptor Family member 2, FGF Receptor Family member 3, FGF Receptor Family member 4, FGF Receptor Family member 5, CDKN2a, APC, RB (retinoblastoma), MEN1, VHL, BRCA1, BRCA2, AR (Androgen Receptor), TSG101, IGF, IGF Receptor, Igf1 variant 1, Igf1 variant 2, Igf1 variant 3, Igf1 variant 4, Igf2 variant 1, Igf2 variant 2, Igf2 variant 3, Igf1 Receptor, Igf2 Receptor, Bax, Bcl2, caspase 1, caspase 2, caspase 3, caspase 4, caspase 6, caspase 7, caspase 8, caspase 9, caspase 12, Kras, or Ape).

The coding nucleic acid sequence of libraries described herein may encode a protein of interest for treatment of age-related macular degeneration (e.g., Aber, Ccl2, Cc2, cp (ceruloplasmin), Timp3, cathepsin D, or V1dlr)

The coding nucleic acid sequence of libraries described herein may encode a protein of interest for treatment of schizophrenia (e.g., Neuregulin (Nrgl), Erb4, Complexin-1 (Cplx1), Tph1 Tryptophan hydroxylase, Tph2 Tryptophan hydroxylase 2, Neurexin 1, GSK3, GSK3a, GSK3b, 5-HIT (Slc6a4), COMT, DRD (Drdla), SLC6A3, DAOA, DTNBPI, or Dao (Daol)).

The coding nucleic acid sequence of libraries described herein may encode a protein of interest for treatment of trinucleotide repeat disorders (e.g., HTT, SBMA/SMAXI/AR, FXN/X25, ATX3, ATXNI and ATXN2, DMPK, Atrophin-1 and Atnl, CBP, VLDLR, Atxn7, or Atxn10).

The coding nucleic acid sequence of libraries described herein may encode a protein of interest for treatment of fragile X syndrome (e.g., FMR2, FXRI, FXR2, or mGLUR5).

The coding nucleic acid sequence of libraries described herein may encode a protein of interest for treatment of secretase related disorders (e.g., APH-1 (alpha and beta), Presenilin (Psenl), nicastrin (Ncstn), PEN-2).

The coding nucleic acid sequence of libraries described herein may encode a protein of interest for treatment of ALS (e.g., SOD1, ALS2, STEX, FUS, TARD BP, or VEGF (VEGF-a, VEGF-b, VEGF-c)).

The coding nucleic acid sequence of libraries described herein may encode a protein of interest for treatment of autism (e.g., Mecp2, BZRAP1, MDGA2, Sema5A, or Neurexin 1).

The coding nucleic acid sequence of libraries described herein may encode a protein of interest for treatment of Alzheimer's disease (e.g., E1, CHIP, UCH, UBB, Tau, LRP, PICALM, Clusterin, PS1, SORL1, CR1, V1dlr, Ubal, Uba3, CHIP28 (Aqp1, Aquaporin 1), Uchl1, Uchl3, APP).

The coding nucleic acid sequence of libraries described herein may encode a protein of interest for treatment of inflammation (e.g., IL-10, IL-1 (IL-Ia, IL-Ib), IL-13, IL-17 (IL-17a (CTLA8), IL-17b, IL-17c, IL-17d, IL-171), 11-23, Cx3crl, ptpn22, TNFa, NOD2/CARD15 for IBD, IL-6, IL-12 (IL-12a, IL-12b), CTLA4, Cx3cll, JAK3, JAKL, DCLREIC, ARTEMIS, SCIDA, RAG1, RAG2, ADA, PTPRC, CD45, LCA, IL7R, CD3D, T3D, IL2RG, SCIDXI, SCIDX, or IMD4).

The coding nucleic acid sequence of libraries described herein may encode a protein of interest for treatment of Parkinson's Disease (e.g., x-Synuclein, DJ-1, LRRK2, Parkin, PINK1).

The coding nucleic acid sequence of libraries described herein may encode a protein of interest (e.g., CRAN1, CDA1, RPS19, DBA, PKLR, PK1, NT5C3, UMPH1, PSNI, RHAG, RH50A, NRAMP2, SPTB, ALAS2, ANH1, ASB, ABCB7, ABC7, ASAT, TAPBP, TPSN, TAP2, ABCB3, PSF2, RING11, MHC2TA, C₂TA, RFX5, RFXAP, RFX5, TBXA2R, P2RX1, P2X1, HF1, CFH, HUS, MCFD2, FANCA, FAC A, FA1, FA, FA A, FAAP95, FAAP90, FLJ34064, FANCB, FANCC, FACC, BRCA2, FANCDI, FANCD2, FANCD, FACD, FAD, FANCE, FACE, FANCF, XRCC9, FANCG, BR1PI, BACH1, FANCJ, PHF9, FANCL, FANCM, KIAA1596, PRF1, HPLH2, UNC13D, MUNC13-4, HPLH3, HLH3, FHL3, F8, FSC, PI, ATT, F5, ITGB2, CD18, LCAMB, LAD, EIF2B1, EIF2BA, EIF2B2, EIF2B3, EIF2B5, LVWM, CACH, CLE, EIF2B4, HBB, HBA2, HBB, HBD, LCRB, HBA1) for treatment of blood and coagulation disorders (e.g., anemia, bare lymphocyte syndrome, bleeding disorders, hemophagocytic lymphohistiocytosis disorders, hemophilia A, hemophilia B, hemorrhagic disorders, leukocyte deficiencies and disorders, sickle cell anemia, or thalassemia).

The coding nucleic acid sequence of libraries described herein may encode a protein of interest for treatment of B-cell non-Hodgkin lymphoma or leukemia (e.g., BCL7A, BCL7, ALI, TCL5, SCL, TAL2, FLT3, NBS1, NBS, ZNFN1AI, 1KI, LYF1, HOXD4, HOX4B, BCR, CML, PHL, ALL, ARNT, KRAS2, RASK2, GMPS, AFIO, ARHGEF12, LARG, KIAA0382, CALM, CLTH, CEBPA, CEBP, CHIC2, BTL, FLT3, KIT, PBT, LPP, NPMI, NUP214, D9S46E, CAN, CAIN, RUNXI, CBFA2, AML1, WHSClLI, NSD3, FLT3, AF1Q, NPMI, NUMA1, ZNF145, PLZF, PML, MYL, STAT5B, AF1Q, CALM, CLTH, ARL 11, ARLTS1, P2RX7, P2X7, BCR, CML, PHL, ALL, GRAF, NF1, VRNF, WSS, NFNS, PTPNII, PTP2C, SHP2, NS1, BCL2, CCND1, PRAD1, BCL1, TCRA, GATA1, GF1, ERYF1, NFE1, ABLI, NQO1, DIA4, NMOR1, NUP214, D9S46E, CAN, CAIN).

The coding nucleic acid sequence of libraries described herein may encode a protein of interest for treatment of immune related diseases and disorders (e.g., KIR3DL1, NKAT3, NKB1, AMB11, K1R3DS1, IFNG, CXCL12, TNFRSF6, APT1, FAS, CD95, ALPS1A, IL2RG, SCIDX1, —SCIDX, IMD4, CCL5, SCYA5, D17S136E, TCP228, IL10, CSIF, CMKBR2, CCR2, CMKBR5, CCCKR5 (CCR5), CD3E, CD3G, AICDA, AID, HIGM2, TNFRSF5, CD40, UNG, DGU, HIGM4, TNFSFS, CD40LG, HIGM1, IGM, FOXP3, IPEX, AIID, XPID, PIDX, TNFRSF14B, or TACI).

The coding nucleic acid sequence of libraries described herein may encode a protein of interest for treatment of metabolic, liver, kidney and protein diseases and disorders (e.g., TTR, PALB, APOA1, APP, AAA, CVAP, ADI, GSN, FGA, LYZ, TTR, PALB, KRT18, KRT8, CIRH1A, NAIC, TEX292, KIAA1988, CFTR, ABCC7, CF, MRP7, SLC2A2, GLUT2, G6PC, G6PT, G6PT1, GAA, LAMP2, LAMPB, AGL, GDE, GBE1, GYS2, PYGL, PFKM, TCF1, HNF1A, MODY3, —SCOD1, SCOl, CTNNB1, PDGFRL, PDGRL, PRLTS, AX1NI, AXIN, CTNNB1, TP53, P53, LFS1, IGF2R, MPRI, MET, CASP8, MCH5, UMOD, HNFJ, FJHN, MCKD2, ADMCKD2, PAH, PKU1, QDPR, DHPR, PTS, FCYT, PKHD1, ARPKD, PKD1, PKD2, PKD4, PKDTS, PRKCSH, G19P1, PCLD, or SEC63).

The coding nucleic acid sequence of libraries described herein may encode a protein of interest for treatment of muscular/skeletal diseases and disorders (e.g., DMD, BMD, MYF6, LMNA, LMN1, EMD2, FPLD, CMDIA, HGPS, LGMDIB, LMNA, LMNI, EMD2, FPLD, CMDIA, FSHMD1A, FSHD1A, FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, —SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDCIC, LGMD21, TTN, CMD1G, TMD, LGMD2J, POMT1, CAV3, LGMD 1C, SEPN1, SELN, RSMD 1, PLEC1, PLTN, EBS1, LRP5, BMND1, LRP7, LR3, OPPG, VBCH2, CLCN7, CLC7, OPTA2, OSTMI, GL, TCIRG1, TIRC7, OC116, OPTB1, VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, or SMARD1).

The coding nucleic acid sequence of libraries described herein may encode a protein of interest for treatment of neurological and neuronal diseases and disorders (e.g., SOD1, ALS2, STEX, FUS, TARDBP, VEGF (VEGF-a, VEGF-b, VEGF-c), APP, AAA, CVAP, ADI, APOE, AD2, PSEN2, AD4, STM2, APBB2, FE65LI, NOS3, PLAU, URK, ACE, DCPI, ACEI, MPO, PACIPI, PAXIPIL, PTIP, A2M, BLMH, BMH, PSEN1, AD3, Mecp2, BZRAP1, MDGA2, Sema5A, Neurexin 1, GLOl, MECP2, RTT, PPMX, MRX16, MRX79, NLGN3, NLGN4, KIAA1260, AUTSX2, FMR2, FXR1, FXR2, mGLUR5, HD, IT15, PRNP, PRIP, JPH3, JP3, HDL2, TBP, —SCA17, NR4A2, NURR1, NOT, TINUR, SNCAIP, TBP, SCA17, SNCA, NACP, PARK1, PARK4, DJI, PARK7, LRRK2, PARK8, PINK1, PARK6, UCHL1, PARK5, SNCA, NACP, PARK1, PARK4, PRKN, PARK2, PDJ, DBH, NDUFV2, MECP2, RTT, PPMX, MRX16, MRX79, CDKL5, STK9, MECP2, RTT, PPMX, MRX16,MRX79, x-Synuclein, DJ-1, Neuregulin-1 (Nrgl), Erb4, Complexin-1 (Cplx1), Tph1 Tryptophan hydroxylase, Tph2, Tryptophan hydroxylase 2, Neurexin 1, GSK3, GSK3a, GSK3b, 5-HTT (Slc6a4), CONT, DRD (Drdla), SLC6A, DAOA, DTNBP1, Dao (Daol), APH-1(alpha and beta), Presenilin (Psenl), Nicastrin, (Ncstn), PEN-2, Nosl, Parpl, Nat1, Nat2, HTT, SBMA/SMAX1/AR, FXN/X25, ATX3, TXN, ATXN2, DMPK, Atrophin-1, Atnl, CBP, VLDLR, Atxn7, or AtxnlO).

The coding nucleic acid sequence of libraries described herein may encode a protein of interest for treatment of ocular diseases and disorders (e.g., Aber, Ccl2, Cc2, cp (ceruloplasmin), Timp3, cathepsin-D, V1dlr, Ccr2, CRYAA, CRYA1, CRYBB2, CRYB2, PITX3, BFSP2, CP49, CP47, CRYAA, CRYAI, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYAI, GJA8, CX50, CAEl, GJA3, CX46, CZP3, CAE3, CCM1, CAM, KRIT1, APOA1, TGFBI, CSD2, CDGG1, CSD, BIGH3, CDG2, TACSTD2, TROP2, MlSI, VSX1, RINX, PPCD, PPD, KTCN, COL8A2, FECD, PPCD2, PIP5K3, CFD, KERA, CNA2, MYOC, TIGR, GLCIA, JO AG, GPOA, OPTN, GLC1E, FIP2, HYPL, NRP, CYPIBI, GLC3A, OPAl, NTG, NPG, CYPIBI, GLC3A, CRB1, RP12, CRX, CORD2, CRD, RPGRIPI, LCA6, CORD9, RPE65, RP20, AIPL1, LCA4, GUCY2D, GUC2D, LCA1, CORD6, RDH12, LCA3, ELOVL4, ADMD, STGD2, STGD3, RDS, RP7, PRPH2, PRPH, AVMD, AOFMD, or VMD2).

This disclosure relates to libraries comprising polynucleotides, wherein the polynucleotide comprises at least one modification, e.g., at least 20%, at least 30%, at least 40%, at least 50%, at least 60%, at least 70%, at least 80%, at least 90%, at least 100% of the bases are modified. This disclosure relates to libraries comprising polynucleotides, wherein the polynucleotide comprises at least one specific base is modified, e.g., adenine, guanine, cytosine, uracil. The modified adenine of this disclosure can comprise at least 20%, at least 30%, at least 40%, at least 50%, at least 60%, at least 70%, at least 80%, at least 90%, or at least 100% of the adenine bases in polynucleotides of this disclosure. The modified guanine of this disclosure can comprise at least 20%, at least 30%, at least 40%, at least 50%, at least 60%, at least 70%, at least 80%, at least 90%, or at least 100% of the guanine bases in polynucleotides of this disclosure. The modified cytosine of this disclosure can comprise at least 20%, at least 30%, at least 40%, at least 50%, at least 60%, at least 70%, at least 80%, at least 90%, or at least 100% of the cytosine bases in polynucleotides of this disclosure. The modified uracil of this disclosure can comprise at least 20%, at least 30%, at least 40%, at least 50%, at least 60%, at least 70%, at least 80%, at least 90%, or at least 100% of the uracil bases in polynucleotides of this disclosure. This disclosure is related to libraries of polynucleotides comprising polynucleotides comprising modified bases, wherein at least one base modification is pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, or N2,N2-dimethyl-6-thio-guanosine.

This disclosure relates to libraries of polynucleotides comprising at least one unique identifier. The unique identifier may be detectable by florescence. A unique identifier of this disclosure may be a fluorescent dye (e.g., fluorescein, TAMRA (carboxytetramethylrhodamine), Cy dyes, Texas red, HEX, JOE, Oregon green, rhodamine 6 G, coumarin, pyrene, or DiOC6 (3,3′-dihexyloxacarbocyanine iodide)).

A unique identifier of this disclosure may be a fluorescent protein (e.g., Green Fluorescent Protein (GFP), Yellow Fluorescent Protein (YFP), Red Fluorescent Protein (RFP), Sirius, excitable blue fluorescent protein (EBFP2), cyan fluorescent protein (CFP), Cerulean, excitable green fluorescent protein (EGFP), excitable yellow fluorescent protein (EYFP), mOrange, mCherry, mPlum, NIR, iRFP, EosFP, PamCherry, Dronpa, Dreiklang, asFP595, mMaple, mGeo, mEos2, Dendra2, psCFP2, and 2,3,5,6-tetracarbazole-4-cyano-pyridine (CPy)).

A unique identifier of this disclosure may be a fluorescent protein, for example, luciferase (e.g., Renilla luciferase, Gaussia luciferase, Nanoluc luciferase, Firefly luciferase, or Click Beetle luciferase).

A unique identifier of this disclosure may be a fluorescent nanoparticle (e.g., carbon dots, graphene quantum dots, gold nanorods, polymer-based nanoparticles, aggregation-induced emission dots, Conjugated Polymer nanoparticles (CP-dots), Gold nanospheres, Gold nano shells, Gold nanocages, or AIE pheromone),

A unique identifier of this disclosure may be a fluorescent lipid (e.g., DiR, DiD, DiO, DiI, Bodipy, or FL-Sphingomyelin).

This disclosure relates to libraries of polynucleotides comprising at least one unique identifier. The unique identifier may be detectable by florescence. At least one unique identifier of this disclosure may be β-galactosidase (β-gal).

This disclosure relates to libraries of polynucleotides comprising at least one unique identifier, wherein the at least one unique identifier may be a quencher molecule (e.g., dimethylaminophenylazobenzoic acid (DABCYL), QSY 7, Cu(II) ion, Dabcyl, QSY 35, BHQ-0, Eclipse, BHQ-1, QSY 9, BHQ-2, ElleQuencher, Iowa Black, QSY 21, and BHQ-3).

This disclosure relates to libraries of polynucleotides comprising at least one unique identifier, wherein the at least one unique identifier may be a fluorophore. Fluorophores of this disclosure may be a quantum dot (e.g., CdSe/ZnS, CdTe/ZnS, CdTe/CdSe, CdSe/ZnTe, CdSe/CdTe/ZnSe, nAs/ZnSe, InAs/CdSe, InAs/InP, Cu:InP/ZnSe, InAsxP1-x/InP/ZnSe, CdS/CdSe, ZnSe/CdSe, ZnSe/InP/ZnS, ZnSe/InP/ZnS, CdTe/ZnSe, QD585, and QD655) and/or an organic small molecule (e.g., 7-dialkyl-amino-4-trifluoromethyl coumarin, rhodamine B, Coumarin 314, Lucifer Yellow CH, florescein, rhodamine 123, BODIPY FL NHS ester, Cy5, Rhodamine 6G, Silicon-rhodamine (SiR), Cy3, Cy5.5, Cy7, Cy2, ATT0655, ATT0680, ATT0700, Nitrobenzoxadiazole (NBD), 1,6-diphenyl-1,3,5-hexatriene (DPH), ABBERIOR™, ALEXA FLUOR™, ATTO™, DYLIGHT FLUOR™, ALEXA FLUOR 647™, and TOPFLUOR™)

This disclosure relates to libraries of polynucleotides comprising at least one unique identifier, wherein the at least one unique identifier may be a radioactive chemical group (e.g., radioactive phosphate).

This disclosure relates to libraries of polynucleotides comprising at least one unique identifier, wherein the at least one unique identifier may be biotin.

This disclosure relates to libraries of polynucleotides comprising at least one unique identifier, wherein the at least one unique identifier may be digoxygenin.

This disclosure relates to libraries of polynucleotides comprising at least one unique identifier, wherein the at least one unique identifier may be dinitrophenyl (DNP).

This disclosure relates to libraries of polynucleotides comprising at least one unique identifier, wherein the at least one unique identifier may be Fluorescein.

This disclosure relates to libraries of polynucleotides comprising at least one unique identifier, wherein the at least one unique identifier may be fucose.

This disclosure relates to libraries of polynucleotides comprising at least one unique identifier, wherein the at least one unique identifier may be an amine.

This disclosure relates to libraries of polynucleotides comprising at least one unique identifier, wherein the at least one unique identifier may be TEXAS RED®.

This disclosure relates to libraries of polynucleotides comprising at least two unique identifiers (e.g., about 2, about 3, about 4, about 5, about 6, about 7, about 8, about 9, about 10, about 11, about 12, about 13, about 14, about 15, about 17, about 19, about 21, about 23, about 25, about 28, about 31, about 34, about 37, about 40, about 44, about 49, about 54, about 59, about 64, about 70, about 76, about 83, about 90, about 100, about 110, about 120, about 130, about 140, about 150, about 160, about 170, about 180, about 190, about 200, about 220, about 240 about 260, about 280, about 300, about 330, about 360, about 390, about 420, about 460, about 500, about 550, about 600, about 650 about 700, about 750, about 800, about 850 about 900, about 950, about 1,000, about 1,100, about 1,200, about 1,300 about 1,400, about 1,500 about 1,600, about 1,700, about 1,800, about 1,900, about 2,000, about 2,200, about 2,400, about 2,600, about 2,800, about 3,000, about 3,300, about 3,600, about 3,900, about 4,200, about 4,600, or about 5,000 unique identifiers).

The unique identifiers of the polynucleotide libraries disclosed herein may be the same.

The unique identifiers of the polynucleotide libraries disclosed herein may be different.

The unique identifiers of the polynucleotide libraries disclosed herein may be between about 2 and about 1000 residues long, e.g., about 2, about 3, about 4, about 5, about 6, about 7, about 8, about 9, about 10, about 12, about 14, about 16, about 18, about 20, about 24, about 28, about 30, about 33, about 36, about 40, about 45, about 50, about 55, about 60, about 65, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, or about 1000 residues long.

This disclosure relates to a composition comprising a population of formulated polynucleotides, wherein each of the formulated polynucleotides is selected from a library of polynucleotides disclosed herein and wherein said polynucleotides are formulated in a delivery vehicle at a ratio of at least 1:1 (PN to DV), said delivery vehicle selected from group consisting of nanoparticles, lipid nanoparticles, micelles, exosomes, targeted lipid nanoparticles, non-lipid nanoparticles, liposomes, viral particles, and polymeric delivery particles.

This disclosure relates to libraries of polynucleotides, wherein the delivery vehicle comprises an exosome.

This disclosure relates to libraries of polynucleotides, wherein the delivery vehicle comprises a micelle.

This disclosure relates to libraries of polynucleotides, wherein the delivery vehicle comprises a non-lipid nanoparticle.

This disclosure relates to libraries of polynucleotides, wherein the delivery vehicle comprises a viral particle.

This disclosure relates to libraries of polynucleotides, wherein the viral particle is an AAV particle.

This disclosure relates to libraries of polynucleotides, wherein the delivery vehicle comprises a polymeric particle.

This disclosure relates to libraries of polynucleotides, wherein the delivery vehicle comprises a nanoparticle. This disclosure relates to libraries of polynucleotides, wherein the nanoparticle comprises a lipid nanoparticle.

This disclosure relates to libraries of polynucleotides, wherein the population of formulated polynucleotides comprises at least two lipid nanoparticles. Libraries of this disclosure of can be a population of formulated polynucleotides comprising at least 2 to at least 10,000 lipid nanoparticles, e.g., at least 2, at least 3, at least 4, at least 5, at least 6, at least 7, at least 8, at least 9, at least 10, at least 12, at least 14, at least 16, at least 18, at least 20, at least 24, at least 28, at least 32, at least 35, at least 38, at least 41, at least 45, at least 49, at least 53, at least 58, at least 63, at least 69, at least 75, at least 80, at least 85, at least 90, at least 95, at least 100, at least 110, at least 120, at least 130, at least 150, at least 170, at least 190, at least 210, at least 240, at least 270, at least 300, at least 360, at least 400, at least 450, at least 500, at least 550, at least 600, at least 650, at least 700, at least 850, at least 900, at least 950, at least 1000, at least 1100, at least 1200, at least 1300, at least 1400, at least 1500, at least 1600, at least 1700, at least 1800, at least 1900, at least 2000, at least 2200, at least 2400, at least 2600, at least 2800, at least 3000, at least 3400, at least 3800, at least 4200, at least 5000, at least 5500, at least 6000, at least 6500, at least 7000, at least 7500, at least 8000, at least 8500, at least 9000, at least 9500, or at least 10,000 lipid nanoparticles.

Lipid nanoparticle formulated polynucleotide libraries of this disclosure may comprise targeted lipid nanoparticles, wherein the targeted lipid nanoparticle may comprise one or more unique identifier. In some aspects, one or more unique identifier of a targeted lipid nanoparticle is detectable by florescence.

In some aspects, one or more unique identifier of a targeted lipid nanoparticle is a fluorescent dye (e.g., fluorescein, TAMRA (carboxytetramethylrhodamine), Cy dyes, Texas red, HEX, JOE, Oregon green, rhodamine 6 G, coumarin, pyrene, or DiOC6 (3,3′-dihexyloxacarbocyanine iodide)).

In some aspects, one or more unique identifier of a targeted lipid nanoparticle is a fluorescent protein (e.g., Green Fluorescent Protein (GFP), Yellow Fluorescent Protein (YFP), Red Fluorescent Protein (RFP), Sirius, excitable blue fluorescent protein (EBFP2), cyan fluorescent protein (CFP), Cerulean, excitable green fluorescent protein (EGFP), excitable yellow fluorescent protein (EYFP), mOrange, mCherry, mPlum, NIR, iRFP, EosFP, PamCherry, Dronpa, Dreiklang, asFP595, mMaple, mGeo, mEos2, Dendra2, psCFP2, and 2,3,5,6-tetracarbazole-4-cyano-pyridine (CPy)).

In some aspects, one or more unique identifier of a targeted lipid nanoparticle is a luciferase (e.g., Renilla luciferase, Gaussia luciferase, Nanoluc luciferase, Firefly luciferase, or Click Beetle luciferase).

In some aspects, one or more unique identifier of a targeted lipid nanoparticle is a fluorescent nanoparticle (e.g., carbon dots, graphene quantum dots, gold nanorods, polymer-based nanoparticles, aggregation-induced emission dots, Conjugated Polymer nanoparticles (CP-dots), Gold nanospheres, Gold nano shells, Gold nanocages, or AIE pheromone).

In some aspects, one or more unique identifier of a targeted lipid nanoparticle is a fluorescent lipid (e.g., DiR, DiD, DiO, DiI, Bodipy, or FL-Sphingomyelin).

In some aspects, one or more unique identifier of a targeted lipid nanoparticle is (3-galactosidase (β-gal).

In some aspects, one or more unique identifier of a targeted lipid nanoparticle is a quencher molecule (e.g., dimethylaminophenylazobenzoic acid (DABCYL), QSY 7, Cu(II) ion, Dabcyl, QSY 35, BHQ-0, Eclipse, BHQ-1, QSY 9, BHQ-2, ElleQuencher, Iowa Black, QSY 21, and BHQ-3).

In some aspects, one or more unique identifier of a targeted lipid nanoparticle is a fluorophore (e.g., a quantum dot (e.g., CdSe/ZnS, CdTe/ZnS, CdTe/CdSe, CdSe/ZnTe, CdSe/CdTe/ZnSe, nAs/ZnSe, InAs/CdSe, InAs/InP, Cu:InP/ZnSe, InAsxP1-x/InP/ZnSe, CdS/CdSe, ZnSe/CdSe, ZnSe/InP/ZnS, ZnSe/InP/ZnS, CdTe/ZnSe, QD585, and QD655), an organic small molecule (e.g., 7-dialkyl-amino-4-trifluoromethyl coumarin, rhodamine B, Coumarin 314, Lucifer Yellow CH, florescein, rhodamine 123, BODIPY FL NHS ester, Cy5, Rhodamine 6G, Silicon-rhodamine (SiR), Cy3, Cy5.5, Cy7, Cy2, ATT0655, ATTO680, ATTO700, Nitrobenzoxadiazole (NBD), 1,6-diphenyl-1,3,5-hexatriene (DPH), ABBERIOR™, ALEXA FLUOR™, ATTO™ DYLIGHT FLUOR™, ALEXA FLUOR 647™, and TOPFLUOR™))

In some aspects, one or more unique identifier of a targeted lipid nanoparticle is a radioactive phosphate.

In some aspects, one or more unique identifier of a targeted lipid nanoparticle is biotin.

In some aspects, one or more unique identifier of a targeted lipid nanoparticle is digoxygenin.

In some aspects, one or more unique identifier of a targeted lipid nanoparticle is dinitrophenyl (DNP).

In some aspects, one or more unique identifier of a targeted lipid nanoparticle is Fluorescein.

In some aspects, one or more unique identifier of a targeted lipid nanoparticle is fucose.

In some aspects, one or more unique identifier of a targeted lipid nanoparticle is an amine.

In some aspects, one or more unique identifier of a targeted lipid nanoparticle is TEXAS RED®.

In some aspects, a targeted lipid nanoparticle comprising one or more unique identifier is attached, appended, bound or incorporated therein one or more targeting moieties selected from the group consisting of glycans, antibodies or fragments thereof, small molecules and peptides.

In some aspects, a targeted lipid nanoparticle has attached, appended, bound or incorporated therein an antibody or fragment thereof (e.g., a monospecific antibody, bispecific antibody, miniaturized antibody, diabody, unibody, intrabody, maxibody, Fab, Fab′, F(ab′)₂, or Fv fragments of an antibody).

In some aspects, targeted lipid nanoparticles of this disclosure can target an organ, tissue, or cell that is not the liver.

In some aspects, targeted lipid nanoparticles of this disclosure can target an organ, tissue, or cell that is not the kidney.

In some aspects, targeted lipid nanoparticles of this disclosure can target an organ, tissue, or cell that is not the spleen.

In some aspects, this disclosure relates to targeted lipid nanoparticles comprising polynucleotides, wherein a higher proportion of the polynucleotides are delivered to a target organ, tissue, or cell than to the liver following administration (e.g., intravenous administration) to a subject.

In some aspects, this disclosure relates to targeted lipid nanoparticles comprising polynucleotides, wherein a higher proportion of the polynucleotides are delivered to a target organ, tissue, or cell than to the kidney following administration (e.g., intravenous administration) to a subject.

In some aspects, this disclosure relates to targeted lipid nanoparticles comprising polynucleotides, wherein a higher proportion of the polynucleotides are delivered to a target organ, tissue, or cell than to the spleen following administration (e.g., intravenous administration) to a subject.

In some aspects, this disclosure relates to targeted lipid nanoparticles comprising polynucleotides, wherein 10 times, 20 times, 30 times, 40 times, 50 times, 60 times, 70 times, 80 times, 90 times, 100 times or more of the polynucleotides are delivered to a target organ, tissue, or cell than to the liver following administration (e.g., intravenous administration) to a subject.

In some aspects, this disclosure relates to targeted lipid nanoparticles comprising polynucleotides, wherein 10 times, 20 times, 30 times, 40 times, 50 times, 60 times, 70 times, 80 times, 90 times, 100 times or more of the polynucleotides are delivered to a target organ, tissue, or cell than to the kidney following administration (e.g., intravenous administration) to a subject.

In some aspects, this disclosure relates to targeted lipid nanoparticles comprising polynucleotides, wherein 10 times, 20 times, 30 times, 40 times, 50 times, 60 times, 70 times, 80 times, 90 times, 100 times or more of the polynucleotides are delivered to a target organ, tissue, or cell than to the spleen following administration (e.g., intravenous administration) to a subject.

This disclosure relates to a method of validating/determining the cellular tropism of a candidate targeting system, comprising
a. contacting a non-human mammal with said candidate targeting system, said candidate targeting system comprising the composition of any targeting system of this disclosure, and
b. determining the cellular localization of the candidate targeting system by detecting for the presence of one or more of said unique identifiers in a plurality of tissues or organs.

This disclosure relates to a method of validating/determining the cellular tropism of a candidate targeting system, wherein the candidate targeting system comprises lipid nanoparticles.

This disclosure relates to a method of validating/determining the cellular tropism of a candidate targeting system, wherein a unique identifier is detected determine the localization of the polynucleotide.

This disclosure relates to a method of validating/determining the cellular tropism of a candidate targeting system, wherein a unique identifier is detected determine the localization of the formulation.

This disclosure relates to a method of validating/determining the cellular tropism of a candidate targeting system, wherein unique identifier detected determine the localization of the formulation polynucleotide.

This disclosure relates to a method of validating/determining the cellular tropism of a candidate targeting system, wherein the detection is by nuclear imaging (e.g., X-ray, magnetic resonance imaging (MRI), functional magnetic resonance imaging (fMRI), nuclear magnetic resonance imaging, computed tomography (CT), positron emission tomography (PET), single-photon emission computed tomography (SPECT), or absorption imaging).

This disclosure relates to a method of validating/determining the cellular tropism of a candidate targeting system, wherein the detection is by optical imaging (e.g., visible light microscopy, Raman spectroscopy, fluorescence microscopy, bioluminescence imaging (BLI), or optical coherence tomography).

This disclosure relates to a method of validating/determining the cellular tropism of a candidate targeting system, wherein the detection is by visible fluorescence microscopy (e.g., confocal fluorescence microscopy, fluorescence reflectance imaging, fluorescence molecular tomographic imaging, or Forster Resonance Energy Transfer (FRET)).

This disclosure relates to a method of validating/determining the cellular tropism of a candidate targeting system, wherein the detection is by bioluminescence imaging (BLI).

This disclosure relates to a method of validating/determining the cellular tropism of a candidate targeting system, wherein the detection is by nucleotide sequencing.

This disclosure relates to a method of validating/determining the cellular tropism of a candidate targeting system, wherein the detection is by cell sorting techniques (e.g., magnetic beads, flow cytometry, cleavage of peptide with LC-MS/MS, or Fluorescence-activated Cell Sorting (FACS)).

This disclosure relates to a method of validating/determining the cellular tropism of a candidate targeting system, further comprising
a. obtaining one or more cells from the one or more organs or tissues of the subject,
b. optionally amplifying one or more unique identifiers in the one or more cells, and
c. identifying the one or more molecules associated with the unique identifiers in the one or more cells from the one or more organs or tissues of the subject, thereby identifying one or more targeted nanoparticles suitable for delivery to one or more tissues or organs of the subject.

This disclosure relates to methods of validating/determining the cellular tropism of a candidate targeting system in tissues, wherein tissues are adrenal medulla, adult fibrous tissue, blood vessels, bone, breast, bronchial lining, carotid body, cartilage, connective tissue, embryonic (myxomatous) fibrous tissue, epithelial, epithelium, fat, glandular epithelium (liver, kidney, bile duct), gonads, hematopoietic cells, lymph vessels, lymphoid tissue, meninges, mesothelium, muscle, nerve sheath, nervous, notochord, ovary, pancreas, parathyroid, pituitary, placenta, renal anlage, smooth muscle, stomach and intestines, stratified squamous, striated muscle, stroma, testis, thyroid, and transitional epithelium. As a non-limiting example, the tissue is connective tissue.

This disclosure relates to methods of validating/determining the cellular tropism of a candidate targeting system in organs, wherein the organs are anal canal, arteries, ascending colon, bladder, bone marrow, brain, bronchi, bronchioles, bulbourethral glands, capillaries, cecum, cerebellum, cerebral hemispheres, cerebrum, cervix, choroid plexus, clitoris, cranial nerves, descending colon, diencephalon, duodenum, ear, enteric nervous system, epididymis, esophagus, external reproductive organs, fallopian tubes, gallbladder, ganglia, gustatory, gut-associated lymphoid tissue, heart, ileum, internal reproductive organs, interstitium, jejunum, joints, kidneys, large intestine, larynx, ligaments, liver, lungs, lymph node, lymphatic vessel, mammary glands, medulla oblongata, mesentery, midbrain, mouth, muscles of breathing, nasal cavity, nerves, olfactory, ovaries, pancreas, parotid glands, penis, pharynx, placenta, pons, prostate, rectum, salivary glands, scrotum, seminal vesicles, sigmoid colon, skeleton, skin, small intestine, spinal nerves, spleen, stomach, subcutaneous tissue, sublingual glands, submandibular glands, teeth, tendons, testes, the brainstem, the spinal cord, the ventricular system, thymus, tongue, tonsils, trachea, transverse colon, ureter, urethra, uterus, vagina, vas deferens, veins, or vulva.

This disclosure relates to a method of delivering a therapeutic nucleic acid to a specific cellular location or site, comprising administering to a subject a therapeutic nucleic acid formulated in a delivery vehicle having validated tropism for said specific cellular location or site.

This disclosure relates to a method of administering to a subject a therapeutic nucleic acid formulated in a delivery vehicle, wherein the administration may be enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, or spinal.

This disclosure relates to a method of identifying a treatment for a specific patient or patient population, the method comprising
a. obtaining a sample from the patient or members of the patient population, said sample containing one or more cells;
b. contacting said sample with one or more candidate targeting systems, said candidate targeting systems being targeting systems of this disclosure,
c. assaying for the presence of one or more of said unique identifiers, tags or labels in said one or more cells in said sample; and
d. identifying said unique identifiers, tags or labels in the one or more cells, thereby correlating the specific candidate targeting system with a cell-specific tropism indicator for the patient or patient population.

This disclosure relates to a method of identifying a treatment for a specific patient or patient population, the method comprising obtaining a sample, wherein said sample can be a tissue sample.

This disclosure relates to a method of identifying a treatment for a specific patient or patient population, the method comprising obtaining a sample, wherein said sample can be a biopsy sample.

This disclosure relates to a method of identifying a treatment for a specific patient or patient population, the method comprising obtaining a sample, wherein said sample can be a blood sample.

This disclosure relates to libraries comprising polynucleotides, wherein the polynucleotides may be small RNA.

This disclosure relates to libraries comprising polynucleotides, wherein the polynucleotides may be microRNA.

This disclosure relates to libraries comprising polynucleotides, wherein the polynucleotides may be transfer RNA.

This disclosure relates to libraries comprising polynucleotides, wherein the polynucleotides are RNA and comprise a regulatory element.

This disclosure relates to libraries comprising polynucleotides, wherein the polynucleotides are RNA and comprise at least one masking agent.

This disclosure relates to libraries comprising polynucleotides, wherein the polynucleotides are RNA, wherein the RNA is produced using in vitro transcription.

This disclosure relates to libraries comprising polynucleotides, wherein the polynucleotides are RNA, wherein the polynucleotide comprises a termination element, wherein the termination element comprises at least one stop codon.

This disclosure relates to libraries comprising polynucleotides, wherein the polynucleotides comprise a payload sequence region comprising a non-coding nucleic acid sequence.

This disclosure relates to libraries comprising polynucleotides, wherein the polynucleotides comprise a payload sequence region comprises a coding nucleic acid sequence.

### VIII. DEFINITIONS

A and An: The articles “a” and “an” are used herein to refer to one or to more than one (i.e., to at least one) of the grammatical object of the article. By way of example, “an element” means one element or more than one element.

About: “About” as used herein when referring to a measurable value such as an amount, a temporal duration, and the like, is meant to encompass variations of 10% from the specified value, as such variations are appropriate to perform the disclosed methods.

Antibodies: As used herein, the term “antibody” is referred to in the broadest sense and specifically covers various embodiments including, but not limited to monoclonal antibodies, polyclonal antibodies, multispecific antibodies (e.g., bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.). Non-limiting examples of antibodies or fragments thereof include V_{H} and V_{L} domains, scFvs, Fab, Fab′, F(ab′)2, Fv fragment, diabodies, linear antibodies, single chain antibody molecules, multispecific antibodies, bispecific antibodies, intrabodies, monoclonal antibodies, polyclonal antibodies, humanized antibodies, codon-optimized antibodies, tandem scFv antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, antibodies to senescent cells, antibodies to conformers, antibodies to disease specific epitopes, or antibodies to innate defense molecules.

Associated: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Biodistribution: As used herein, the term “biodistribution” refers to the concept of tracking where compound(s) of interest travel in a subject. Determining the biodistribution of a compound of interest allows for the determination of the organ system(s) that a compound of interest localizes in, and the relative amounts/concentrations of this localization.

Cargo: As used herein, the term “cargo” or “payload” can refer to one or more molecules or structures encompassed in a delivery vehicle for delivery to or into a cell or tissue. Non-limiting examples of cargo can include a nucleic acid, a polypeptide, a peptide, a protein, a liposome, a label, a tag, a small chemical molecule, a large biological molecule, and any combinations thereof.

Chimeric Antigen Receptors (CARs): As used herein, the term “chimeric antigen receptor” or “CAR” refers to an artificial chimeric protein comprising at least one antigen specific targeting region (ASTR), a transmembrane domain and an intracellular signaling domain, wherein the antigen specific targeting region comprises a full-length antibody or a fragment thereof. Any molecule that is capable of binding a target antigen with high affinity can be used in the ASTR of a CAR. The CAR may optionally have an extracellular spacer domain and/or a co-stimulatory domain. A CAR may also be used to generate a cytotoxic cell carrying the CAR.

Circular RNA: As used herein, the term “circular RNA” or “circRNA” or “oRNA” are used interchangeably and refer to a RNA that forms a circular structure through covalent or non-covalent bonds.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Delivery: As used herein, “delivery” refers to the act or manner of delivering a compound, substance, entity, moiety, cargo or payload.

DNA and RNA: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (e.g., ssRNA or ssDNA, respectively) or multi-stranded (e.g., double stranded, e.g., dsRNA and dsDNA, respectively). The term “mRNA” or “messenger RNA”, as used herein, refers to a single stranded RNA that encodes the amino acid sequence of one or more polypeptide chains.

Encapsulate: As used herein, the term “encapsulate” means to enclose, surround or encase.

Enhance expression ofa gene: As used herein, the phrase “add-back” or “enhance expression of a gene” means to cause an increase in the amount of an expression product of the gene. The expression product can be an RNA transcribed from the gene (e.g., an mRNA) or a polypeptide translated from an mRNA transcribed from the gene. Typically, an increase in the level of an mRNA results in an increase in the level of a polypeptide translated therefrom. The level of expression may be determined using standard techniques for measuring mRNA or protein.

Exosomes: As used herein, “exosome” is a vesicle secreted by mammalian cells or a complex involved in RNA degradation.

Formulation: As used herein, a “formulation” includes at least one compound, substance, entity, moiety, cargo or payload and a delivery agent.

Fragment: A “fragment,” as used herein, refers to a portion. For example, fragments of proteins may comprise polypeptides obtained by digesting full-length protein isolated from cultured cells.

Homology: As used herein, the terms “homology” or “sequence identity” refer to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Inactive Ingredient: As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA).

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “knock-down” or “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Modified: As used herein “modified” refers to a changed state or structure of a molecule. Molecules may be modified in many ways including chemically, structurally, and functionally.

Nanoparticle: As used herein, the term “nanoparticle” refers to any particle ranging in size from 10-1,000 nm.

LNP: As used herein, a “lipid nanoparticle” or “LNP” is a composition comprising one or more lipids. LNPs are typically sized on the order of micrometers or smaller and may include a lipid bilayer, and preferably have an average size of less than 1 micrometer.

PEG lipid: As used herein, a “PEG lipid” or “PEGylated lipid” refers to a lipid comprising a polyethylene glycol component.

Ionizable lipid: As used herein, the interchangeable terms “ionizable lipid” and “cationic lipid” refer to a lipid capable of either positively charged or neutrally charged, depending on pH. Exemplary ionizable lipids comprise one or more fatty acid or fatty aliphatic chains and one or more moieties capable of bearing a positive charge. In preferred embodiments, the moiety capable of bearing the positive charge is a protonatable amine group. Preferred ionizable or cationic lipids are ionizable such that they can exist in a positively charged or neutral form depending on pH of the surrounding environment. Preferable ionizable lipids are protonated to form a cation at acidic physiological pH (about pH 4) and are neutral at neutral pH (pH 7).

Peptide: As used herein, “peptide” is less than or equal to 50 amino acids long, e.g., about 5, 10, 15, 20, 25, 30, 35, 40, 45, or 50 amino acids long.

Pharmaceutical Composition: As used herein the term “pharmaceutical composition” refers to compositions comprising at least one active ingredient and optionally one or more pharmaceutically acceptable excipients.

Prevent: As used herein, the terms “prevent”, “preventing”, and “prevention” of a disease or disorder refer to an action, for example, administration of an LNP encapsulating a cargo, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting a change of 5%, 10%, 15%, 20%, 30%, 40%, 50%, 60%, 70%, 80%, 90% or greater as compared to a control level. Such terms can include but do not necessarily include complete elimination.

Subject: As used herein, the terms “subject” and “patient” refer to an animal, preferably a mammal, including but not limited to, humans, rodents, such as mice and rats, non-human primates, and other laboratory animals.

Treatment: As used herein, the terms “treatment”, “treat”, or “treating” refer to a method of reducing the effects of a disease or condition or symptom of the disease or condition. Thus, in the disclosed method, treatment can refer to a 5%, 10%, 15%, 20%, 30%, 40%, 50%, 70%, 80%, 90%, or 100%.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise viral parent or reference sequence. Such parent or reference viral sequences may serve as an original, second, third or subsequent sequence for engineering vectors. In non-limiting examples, such parent or reference viral sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These viral sequences may serve as either the “donor” sequence of one or more codons (at the nucleic acid level) or amino acids (at the polypeptide level) or “acceptor” sequences of one or more codons (at the nucleic acid level) or amino acids (at the polypeptide level). The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

As used herein, the following abbreviations and initialisms have the indicated meanings:

| | |
|---|---|
| MC3 | 4-(dimethylamino)-butanoic acid, (10Z,13Z)-1-(9Z,12Z)-9,12-octadecadien-1- |
| | y1-10,13-nonadecadien-1-yl ester |
| DSPC | 1,2-distearoyl-sn-glycero-3-phosphocholine |
| DMG | 1,2-Dimyristoyl-rac-glycero-3-methanol |
| DOMG-PEG | R-3-[ω-methoxy-poly(ethyleneglycol))carbamoyl)]-1,2-dimyristyloxypropyl- |
| | 3-amine |
| DLPE | 1,2-Dilauroyl-sn-Glycero-3-Phosphoethanolamine |
| DMPE | 1,2-Dimyristoyl-sn-Glycero-3-Phosphoethanolamine |
| DPPC | 1,2-dipalmitoyl-sn-glycero-3-phosphocholine |
| DSPE | 1,2-distearoyl-sn-glycero-3-phosphoethanolamine |
| DDAB | Didodecyldimethylammonium bromide |
| EPC | 1,2-dioleoyl-sn-glycero-3-ethylphosphocholine |
| 14PA | 1,2-dimyristoyl-sn-glycero-3-phosphate |
| 18BMP | bis(monooleoylglycero)phosphate |
| DODAP | 1,2-dioleoyl-3-dimethylammonium-propane |
| DOTAP | 1,2-dioleoyl-3-trimethylammonium-propane |
| C12-200 | 1,1′-((2-(4-(2-((2-(bis(2-hydroxydodecyl)amino)ethyl)(2- |
| | hydroxydodecyl)amino)ethyl)piperazin-1-yl)ethyl)azanediyl)bis(dodecan-2-ol) |

Throughout the disclosure, chemical substituents described in Markush structures are represented by variables. Where a variable is given multiple definitions as applied to different Markush formulas in different sections of the disclosure, it is to be understood that each definition should only apply to the applicable formula in the appropriate section of the disclosure.

Throughout the disclosure, all embodiments utilizing the term “comprising” should be understood to also contemplate alternative embodiments wherein the term “comprising” is substituted with the terms “consisting of” and “consisting essentially of”.

The present disclosure is further illustrated by the following non-limiting examples.

### IX. EXAMPLES

### Example 1: Evaluation of Candidate LNP Targeting Systems

A library of candidate targeting systems is prepared where the candidate targeting systems comprise at least one identifier sequence or moiety in the formulation and at least one identifier sequence and/or payload in the nucleic acid construct.

### Candidate Targeting System Generation

A population of lipid nanoparticle (LNP) formulations are generated where the cationic lipid component is labeled with at least one identifier sequence or moiety. The LNP formulations that are generated may include LNPs where (a) the components are the same for all formulations and the molar ratios of the components are the same for all the LNP formulations, (b) the components are the same for all formulations but the molar ratios of the components are different for all the LNP formulations, or (c) the components are different for the LNP formulations. Each of the different LNP formulation can include different identifier sequence or moiety in order to track targeting system after administration. Nucleic acid constructs including at least one identifier sequence or payload (e.g., a reporter gene) is generated and formulated in the population of LNPs in order to create candidate targeting systems to be administered to a subject.

### Screening and Validation of Candidate Targeting Systems

The candidate targeting systems are then administered into a subject at a pre-determined dose and dosing interval. After administration the entire subject or a region of the subject is screened to determine the location of the LNP formation and/or the payload of the benchmark construct. The subject can be scanned by various methods known in the art including positron emission tomography (PET) and computed tomography (CT) utilizing the ⁶⁴Cu radiolabel. The localization of the LNP formation and/or the payload the will be determined by visual inspection of the PET images for areas with the greatest concentration of ⁶⁴Cu and anatomical position of PET results will be confirmed using the results of the CT scan. The scan can be repeated in order to determine if the localization changes over time.

At the desired time points, samples will be taken from the areas of the subject displaying localization of the LNP and/or the payload in the whole animal localization screening performed above for higher resolution screening of the distribution.

The samples can then be prepared for Fluorescence-activated Cell Sorting (FACS) via the directions supplied with the cell sorter. These populations of cells are then prepared for deep sequencing to determine the presence and identity of the payload and/or identifier sequence.

These results from the screening of the LNP library will provide the tropism of the LNP formulation and the nucleic acid construct that was administered to the subject.

### Example 2: LNP GMO Model

A CRE library is prepared with a unique identifier on the oRNA. Each LNP can have a unique ionizable lipid. A mouse may be transfected with a CRE/Lox RFP gene. Red blood cells may also be sorted to sequence the subsequent unique identifier on the oRNA.

### Example 2a: Generation of Modified Cre-Recombinase

Modified CRE-recombinase may be created in a variety of methods. For example, as described in Kaczmarczyk SJ, Green JE. A single vector containing modified cre recombinase and LOX recombination sequences for inducible tissue-specific amplification of gene expression. Nucleic Acids Research. 2001 June;29(12):E56-6. DOI: 10.1093/nar/29.12.e56. PMID: 11410679; PMCID: PMC55755, which is incorporated herein in its entirety.

Alteration of the 5′ region and introduction of a Kozak consensus translational start site, LOX sites and intron.

Plasmid pBS185 (Life Technologies) containing the CMV enhancer/promoter driving expression of wild-type cre was digested with EcoRI, partially digested with HindIII, blunt-ended and religated to remove a 1.0 kb fragment containing a KpnI site generating pCMV-cre-del (p205). Plasmid p205 is digested with XhoI and BssHII to remove a 5′ region of cre. A 5′ portion of cre is PCR amplified using OLN C (5′-CGC-CAG-AAT-TCC-AAA-ATT-TGC-CTG-CAT-TAC-CGG-TCG-ATG-CAA-CG-3′ (SEQ ID NO: 34)) and OLN D (5′-GAC-CGC-GCG-CCT-GCA-GAT-ATA-GAA-GAT-AAT-CGC-GAA-CAT-CTT-3′ (SEQ ID NO: 35)), which contains an internal PstI restriction site generated by a silent mutation and digested with EcoRI and BssHII. The p205 XhoI/BssHII fragment, cre amplification product and the synthetic double-stranded oligonucleotides OLN A (5′-CTG-AGC-GGC-CGC-CTA-GGC-CCA-TGG-CGA-ATT-TAC-TGA-CGG-TAC-CAG-3′ (SEQ ID NO: 36)) and OLN B (5′-AAT-TCT-GGT-ACC-GTC-AGT-AAA-TTC-GCC-ATG-GCC-TAG-GCGGCC-GC-3′ (SEQ ID NO: 37)) are ligated together to form pCMV-cre-K (p209). These modifications may result in the significant shortening of the 5′ untranslated region from 484 to 19 bp, an optimized Kozak translational start site (28) and the introduction of an EcoRI site at nucleotide 509 and a PstI restriction site through a silent T→G mutation at nucleotide 718 within the coding region of cre to allow for additional alterations of cre. These alterations may result in the changes in the amino acid sequence of Cre. The second amino acid is changed from serine to alanine, the eighth amino acid is changed from histidine to proline and codons for glutamic acid and phenylalanine are inserted at amino acid positions 9 and 10. The human P-globin intron from plasmid pCI (Promega) is altered by PCR mutagenesis to place a PstI site at the 5′ end and a BssHII site at the 3′ end using PCR primers OLN E (5′-GCG-ATC-TGC-AGG-TAA-GTA-TCA-AGG-TTA-CAA-GAC-AGG-3′ (SEQ ID NO: 38)) and OLN F (5′-ATA-TGC-GCG-CCT-GTG-GAG-AGA-AAG-GCA-AAG-TGG-AT-3′ (SEQ ID NO: 39)). The 5′ primer E contains a PstI restriction site and the first 35 nt of the P-globin intron (position 953 to 881 in pCI) and the 3′ primer F contains a BssHII restriction site and 26 nt of the 3′ portion of the P-globin intron (position 890 to 965 in pCI). The modified human P-globin intron is cloned into the PstI/BssH II sites of pCMV-cre-K to generate pCMV-CREM (p²10).

Generation of a plasmid containing modified cre recombinase and a single LOX 511 site inserted into the cre coding region.

An aliquot of plasmid pCMV-CREM (p210) is digested with KpnI and another aliquot is digested with EcoRI. Both aliquots are treated with calf intestinal alkaline phosphatase and subsequently digested with ScaI. The ScaI/KpnI fragment containing the CMV promoter and 5′ region of Cre and the ScaI/EcoRI fragment containing the remaining portion of cre are isolated and ligated with a phosphorylated oligonucleotides OLN 135 (5′-CAA-TAA-CTT-CGT-ATA-ATG-TAT-ACT-ATA-CGA-AGT-TAT-TCG-3′ (SEQ ID NO: 40)) and OLN 136 (5′-AAT-TCG-AAT-AAC-TTC-GTA-TAG-TAT-ACA-TTA-TAC-GAA-GTT-ATT-GGT-AC-3′ (SEQ ID NO: 41)) containing KpnI and EcoRI sites at the 5′ and 3′ ends of the LOX 511 sequence to generate pCMV-CREM-L (p218).

Generation of a plasmid containing floxed RFP inserted into the coding sequence of cre recombinase.

Plasmid pDsRedl-N1 (Clontech) is digested with DraIII. An aliquot of this digest is cut with KpnI and the 3.5 kb DraIII/KpnI fragment was isolated. Another aliquot is digested with AgeI and the 1.1 kb DraIII/AgeI fragment is isolated. The two isolated fragments are ligated with oligonucleotides OLN 03 (5′-CAA-TAA-CTT-CGT-ATA-ATG-TAT-ACT-ATA-CGA-AGT-TAT-CTA-GA-3′ (SEQ ID NO: 42)) and OLN 04 (5′-CCG-GTC-TAG-ATA-ACT-TCG-TAT-AGT-ATA-CAT-TAT-ACG-AAG-TTA-TTG-GTA-C-3′ (SEQ ID NO: 43)) containing the LOX 511 sequence (29) flanked by KpnI and AflII restriction sites resulting in the generation of p221. p²²¹ is digested with KpnI and AflII to isolate the fragment containing a single LOX 511 recognition sequence upstream of the RFP gene. pCMV-CREM-L (p218) is digested with KpnI and EcoRI to remove the LOX 511 site. These two fragments are ligated together with the synthetic oligonucleotides OLN 05 (5′-TTA-AGA-ATA-ACT-TCG-TAT-AAT-GTA-TAC-TAT-ACG-AAG-TTA-TTC-G-3′ (SEQ ID NO: 44)) and OLN 06 (5′-AAT-TCG-AAT-AAC-TTC-GTA-TAG-TAT-ACA-TTA-TAC-GAA-GTT-ATT-C-3′ (SEQ ID NO: 45)) containing a LOX 511 site flanked by AflII and EcoRI. The final plasmid product containing the floxed RFP inserted within the coding sequence of cre recombinase is designated pCMV-RFP/CREM (p224).

### Example 3: LNP Non-GMO Model

For a non-GMO Model, the oRNA may be combined with an OX40 ligand and a unique identifier. Each LNP may have a unique ionizable lipid. A rat may be transfected with the library. Disaggregate tissues may be labeled with an anti-human OX40 antibody. Labeled cells are then sorted and sequenced. A surface marker is detectable by fluorescent antibody detection under fluorescence-activated cell sorting.

### Example 3a: Clonin Expression and Purification of Recombinant Human OX40 Receptor

Cloning expression and purification of recombinant human OX40 receptor can be done as described in US patent US20170037137A1, which is incorporated herein in its entirety. cDNA encoding the extracellular domain of human OX40 Receptor is cloned into a pREP4 expression plasmid (Invitrogen) using standard restriction enzyme digestion and ligation. The construct contained a human Fc portion to aid purification. Constructs are sequenced to ensure their correct sequence composition.

Human OX40 Receptor is expressed transiently to produce recombinant protein using Invitrogen's FreeStyle™ CHO—S suspension adapted cell line. Plasmids are transfected into the cells using PEI (polyethylenimine MW 40000) and left to overgrow for a period of 13 days before harvesting the supernatant for purification. Cells are fed during the overgrow process with ActiCHO™ Feeds A and B from GE Healthcare to help boost productivity and promote longevity of the cells. During the overgrow process, samples are taken regularly to monitor cell growth and viability.

The Fc tagged OX40 Receptor protein is purified in a two-step process; firstly, the clarified tissue culture supernatants from the CHO—S expression are purified using Protein G affinity chromatography. The eluted fractions containing the OX40 Receptor protein are then subjected to size exclusion chromatography and assessed for purity by SDS-PAGE analysis and quantified by spectrophotometer reading at OD280 nm.

### Example 4: Barcoded Polynucleotide Synthesis

### Synthesis of DNA Barcodes

Barcoded DNA sequences SEQ ID NOs: 1-11 were purchased from Integrated DNA Technologies. The DNA was synthesized through standard automated solid phase DNA synthesis.

### Synthesis of oRNA mRNA Barcodes

Barcoded circular RNA and mRNA were procured from Orna Therapeutics, Inc. and Vernal Biosciences, Inc., respectively. Circular RNA and mRNA were synthesized through methods well known in the art, such as, the methods described in PCT Application Publication WO2021113777A2, which is incorporated by reference herein, in its entirety.

TABLE 3
Barcode Libraries Synthesized

| Name | Sequence |
|---|---|
| Barcode 1 | GGCTCAAT or GGCUCAAU |
| Barcode 2 | CGACCTAA or CGACCUAA |
| Barcode 3 | TAACGTCG or UAACGUCG |
| Barcode 4 | AAGCATCG or AAGCAUCG |
| Barcode 5 | GCAATTCC or GCAAUUCC |
| Barcode 6 | GCTACAAC or GCUACAAC |
| Barcode 7 | TAGGAGCT or UAGGAGCU |
| Barcode 8 | TGGATGGT or UGGAUGGU |
| Barcode 9 | TACTAGCG or UACUAGCG |
| Barcode 10 | CAGTGCTT or CAGUGCUU |
| Barcode 11 | GATCAAGG or GAUCAAGG |
| DNA1 | TGCCGTGTAATAGTGAGATCTGGAGGCTCAATNNNNNNNNNNTCGT |
| | GACTGGGAAAACCAGAC (SEQ ID NO.: 1) |
| DNA2 | TGCCGTGTAATAGTGAGATCTGGACGACCTAANNNNNNNNNNTCGT |
| | GACTGGGAAAACCAGAC (SEQ ID NO.: 2) |
| DNA3 | TGCCGTGTAATAGTGAGATCTGGATAACGTCGNNNNNNNNNNTCGT |
| | GACTGGGAAAACCAGAC (SEQ ID NO.: 3) |
| DNA4 | TGCCGTGTAATAGTGAGATCTGGAAAGCATCGNNNNNNNNNNTCGT |
| | GACTGGGAAAACCAGAC (SEQ ID NO.: 4) |
| DNA5 | TGCCGTGTAATAGTGAGATCTGGAGCAATTCCNNNNNNNNNNTCGT |
| | GACTGGGAAAACCAGAC (SEQ ID NO.: 5) |
| DNA6 | TGCCGTGTAATAGTGAGATCTGGAGCTACAACNNNNNNNNNNTCGT |
| | GACTGGGAAAACCAGAC (SEQ ID NO.: 6) |
| DNA7 | TGCCGTGTAATAGTGAGATCTGGATAGGAGCTNNNNNNNNNNTCGT |
| | GACTGGGAAAACCAGAC (SEQ ID NO.: 7) |
| DNA8 | TGCCGTGTAATAGTGAGATCTGGATGGATGGTNNNNNNNNNNTCGT |
| | GACTGGGAAAACCAGAC (SEQ ID NO.: 8) |
| DNA9 | TGCCGTGTAATAGTGAGATCTGGATACTAGCGNNNNNNNNNNTCGT |
| | GACTGGGAAAACCAGAC (SEQ ID NO.: 9) |
| DNA10 | TGCCGTGTAATAGTGAGATCTGGACAGTGCTTNNNNNNNNNNTCGT |
| | GACTGGGAAAACCAGAC (SEQ ID NO.: 10) |
| DNA11 | TGCCGTGTAATAGTGAGATCTGGAGATCAAGGNNNNNNNNNNTCGT |
| | GACTGGGAAAACCAGAC (SEQ ID NO.: 11) |
| mRNA1 | GGGAAAUAAGAGAGAAAAGAAGAGUAAGAAGAAAUAUAAGACCC |
| | CGGCGCCGCCACCAUGGAGGACGCCAAGAACAUCAAGAAGGGCCC |
| | UGCGCCAUUCUACCCCCUGGAAGAUGGAACUGCCGGCGAACAGUU |
| | GCACAAGGCCAUGAAGCGCUACGCCUUGGUGCCCGGUACUAUCGC |
| | CUUCACCGACGCCCACAUCGAAGUGGACAUAACCUACGCGGAGUA |
| | CUUCGAAAUGUCCGUGAGACUGGCCGAGGCUAUGAAGAGAUACGG |
| | ACUGAACACCAACCACCGCAUCGUGGUCUGCUCGGAGAACAGCCU |
| | GCAGUUCUUCAUGCCUGUCCUCGGUGCAUUGUUCAUCGGAGUGGC |
| | GGUGGCUCCUGCAAACGACAUCUACAACGAACGCGAACUCCUGAA |
| | CUCCAUGGGCAUCAGCCAGCCGACUGUAGUGUUCGUGAGCAAAAA |
| | GGGACUCCAGAAGAUCCUGAACGUGCAGAAGAAGCUGCCGAUCAU |
| | UCAAAAGAUUAUCAUCAUGGACUCCAAGACCGACUACCAGGGCUU |
| | CCAGUCGAUGUAUACCUUCGUGACCUCCCAUCUUCCGCCGGGGUU |
| | CAAUGAGUACGAUUUCGUGCCUGAGUCUUUCGACCGGGACAAGAC |
| | UAUCGCCCUCAUCAUGAAUAGCUCCGGAUCCACCGGUCUGCCCAA |
| | GGGAGUGGCCCUGCCACAUCGGACCGCCUGCGUGAGAUUCUCACA |
| | CGCCCGGGAUCCCAUCUUCGGAAACCAGAUCAUCCCCGACACCGC |
| | GAUUCUGUCCGUGGUGCCGUUUCACCACGGAUUCGGGAUGUUCAC |
| | CACUCUGGGUUACCUGAUUUGCGGCUUCCGCGUGGUGCUGAUGUA |
| | CCGGUUCGAGGAGGAACUGUUUCUGCGGAGCCUCCAGGAUUACAA |
| | GAUUCAGAGCGCCCUUCUGGUCCCGACUCUCUUCUCGUUCUUUGC |
| | CAAGUCCACACUUAUUGACAAAUACGAUCUCUCCAACCUCCAUGA |
| | AAUUGCCUCAGGAGGGGCUCCGCUGAGCAAGGAGGUCGGAGAGGC |
| | UGUGGCCAAGCGGUUCCACCUACCCGGCAUUAGACAGGGUUACGG |
| | CCUGACGGAAACCACCUCCGCGAUCCUGAUCACUCCGGAGGGCGA |
| | UGACAAGCCUGGAGCUGUGGGGAAGGUCGUUCCAUUCUUUGAAGC |
| | CAAGGUCGUCGACCUGGAUACCGGCAAAACCCUGGGUGUCAACCA |
| | GCGCGGAGAGCUGUGUGUGCGCGGACCUAUGAUUAUGUCCGGCUA |
| | CGUGAACAACCCCGAGGCCACUAACGCCCUCAUCGACAAGGACGG |
| | CUGGCUGCACUCCGGGGACAUCGCCUACUGGGACGAGGACGAGCA |
| | CUUCUUCAUUGUGGACCGGCUGAAGUCCCUUAUCAAGUAUAAGGG |
| | CUACCAAGUCGCACCGGCUGAACUCGAAUCAAUCUUGCUGCAACA |
| | CCCCAACAUCUUCGACGCCGGAGUGGCAGGACUGCCUGAUGACGA |
| | CGCGGGAGAACUCCCAGCAGCCGUGGUGGUGCUGGAACAUGGCAA |
| | AACCAUGACCGAGAAGGAAAUCGUGGAUUACGUGGCGUCGCAAGU |
| | CACCACUGCCAAGAAGCUGAGGGGGGGAGUGGUCUUUGUGGAUGA |
| | AGUGCCGAAGGGGCUGACGGGGAAGCUGGACGCCAGGAAGAUUAG |
| | GGAGAUCCUGAUUAAGGCCAAGAAGGGCGGCAAAAUUGCCGUGUA |
| | AUAGUGAGAUCUGGAAACAGCUAUGACCAUGGAGGCUCAAUNNN |
| | NNNNNNNGUCGUGACUGGGAAAACCAGACUAGUGACUGACUAGG |
| | AUCUGGUUACCACUAAACCAGCCUCAAGAACACCCGAAUGGAGUC |
| | UCUAAGCUACAUAAUACCAACUUACACUUACAAAAUGUUGUCCCC |
| | CAAAAUGUAGCCAUUCGUAUCUGCUCCUAAUAAAAAGAAAGUUUC |
| | UUCACAUGCGGCCGCAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAA (SEQ ID NO.: 12) |
| mRNA2 | GGGAAAUAAGAGAGAAAAGAAGAGUAAGAAGAAAUAUAAGACCC |
| | CGGCGCCGCCACCAUGGAGGACGCCAAGAACAUCAAGAAGGGCCC |
| | UGCGCCAUUCUACCCCCUGGAAGAUGGAACUGCCGGCGAACAGUU |
| | GCACAAGGCCAUGAAGCGCUACGCCUUGGUGCCCGGUACUAUCGC |
| | CUUCACCGACGCCCACAUCGAAGUGGACAUAACCUACGCGGAGUA |
| | CUUCGAAAUGUCCGUGAGACUGGCCGAGGCUAUGAAGAGAUACGG |
| | ACUGAACACCAACCACCGCAUCGUGGUCUGCUCGGAGAACAGCCU |
| | GCAGUUCUUCAUGCCUGUCCUCGGUGCAUUGUUCAUCGGAGUGGC |
| | GGUGGCUCCUGCAAACGACAUCUACAACGAACGCGAACUCCUGAA |
| | CUCCAUGGGCAUCAGCCAGCCGACUGUAGUGUUCGUGAGCAAAAA |
| | GGGACUCCAGAAGAUCCUGAACGUGCAGAAGAAGCUGCCGAUCAU |
| | UCAAAAGAUUAUCAUCAUGGACUCCAAGACCGACUACCAGGGCUU |
| | CCAGUCGAUGUAUACCUUCGUGACCUCCCAUCUUCCGCCGGGGUU |
| | CAAUGAGUACGAUUUCGUGCCUGAGUCUUUCGACCGGGACAAGAC |
| | UAUCGCCCUCAUCAUGAAUAGCUCCGGAUCCACCGGUCUGCCCAA |
| | GGGAGUGGCCCUGCCACAUCGGACCGCCUGCGUGAGAUUCUCACA |
| | CGCCCGGGAUCCCAUCUUCGGAAACCAGAUCAUCCCCGACACCGC |
| | GAUUCUGUCCGUGGUGCCGUUUCACCACGGAUUCGGGAUGUUCAC |
| | CACUCUGGGUUACCUGAUUUGCGGCUUCCGCGUGGUGCUGAUGUA |
| | CCGGUUCGAGGAGGAACUGUUUCUGCGGAGCCUCCAGGAUUACAA |
| | GAUUCAGAGCGCCCUUCUGGUCCCGACUCUCUUCUCGUUCUUUGC |
| | CAAGUCCACACUUAUUGACAAAUACGAUCUCUCCAACCUCCAUGA |
| | AAUUGCCUCAGGAGGGGCUCCGCUGAGCAAGGAGGUCGGAGAGGC |
| | UGUGGCCAAGCGGUUCCACCUACCCGGCAUUAGACAGGGUUACGG |
| | CCUGACGGAAACCACCUCCGCGAUCCUGAUCACUCCGGAGGGCGA |
| | UGACAAGCCUGGAGCUGUGGGGAAGGUCGUUCCAUUCUUUGAAGC |
| | CAAGGUCGUCGACCUGGAUACCGGCAAAACCCUGGGUGUCAACCA |
| | GCGCGGAGAGCUGUGUGUGCGCGGACCUAUGAUUAUGUCCGGCUA |
| | CGUGAACAACCCCGAGGCCACUAACGCCCUCAUCGACAAGGACGG |
| | CUGGCUGCACUCCGGGGACAUCGCCUACUGGGACGAGGACGAGCA |
| | CUUCUUCAUUGUGGACCGGCUGAAGUCCCUUAUCAAGUAUAAGGG |
| | CUACCAAGUCGCACCGGCUGAACUCGAAUCAAUCUUGCUGCAACA |
| | CCCCAACAUCUUCGACGCCGGAGUGGCAGGACUGCCUGAUGACGA |
| | CGCGGGAGAACUCCCAGCAGCCGUGGUGGUGCUGGAACAUGGCAA |
| | AACCAUGACCGAGAAGGAAAUCGUGGAUUACGUGGCGUCGCAAGU |
| | CACCACUGCCAAGAAGCUGAGGGGGGGAGUGGUCUUUGUGGAUGA |
| | AGUGCCGAAGGGGCUGACGGGGAAGCUGGACGCCAGGAAGAUUAG |
| | GGAGAUCCUGAUUAAGGCCAAGAAGGGCGGCAAAAUUGCCGUGUA |
| | AUAGUGAGAUCUGGAAACAGCUAUGACCAUGGACGACCUAANNNN |
| | NNNNNNGUCGUGACUGGGAAAACCAGACUAGUGACUGACUAGGA |
| | UCUGGUUACCACUAAACCAGCCUCAAGAACACCCGAAUGGAGUCU |
| | CUAAGCUACAUAAUACCAACUUACACUUACAAAAUGUUGUCCCCC |
| | AAAAUGUAGCCAUUCGUAUCUGCUCCUAAUAAAAAGAAAGUUUCU |
| | UCACAUGCGGCCGCAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAA (SEQ ID NO.: 13) |
| mRNA3 | GGGAAAUAAGAGAGAAAAGAAGAGUAAGAAGAAAUAUAAGACCC |
| | CGGCGCCGCCACCAUGGAGGACGCCAAGAACAUCAAGAAGGGCCC |
| | UGCGCCAUUCUACCCCCUGGAAGAUGGAACUGCCGGCGAACAGUU |
| | GCACAAGGCCAUGAAGCGCUACGCCUUGGUGCCCGGUACUAUCGC |
| | CUUCACCGACGCCCACAUCGAAGUGGACAUAACCUACGCGGAGUA |
| | CUUCGAAAUGUCCGUGAGACUGGCCGAGGCUAUGAAGAGAUACGG |
| | ACUGAACACCAACCACCGCAUCGUGGUCUGCUCGGAGAACAGCCU |
| | GCAGUUCUUCAUGCCUGUCCUCGGUGCAUUGUUCAUCGGAGUGGC |
| | GGUGGCUCCUGCAAACGACAUCUACAACGAACGCGAACUCCUGAA |
| | CUCCAUGGGCAUCAGCCAGCCGACUGUAGUGUUCGUGAGCAAAAA |
| | GGGACUCCAGAAGAUCCUGAACGUGCAGAAGAAGCUGCCGAUCAU |
| | UCAAAAGAUUAUCAUCAUGGACUCCAAGACCGACUACCAGGGCUU |
| | CCAGUCGAUGUAUACCUUCGUGACCUCCCAUCUUCCGCCGGGGUU |
| | CAAUGAGUACGAUUUCGUGCCUGAGUCUUUCGACCGGGACAAGAC |
| | UAUCGCCCUCAUCAUGAAUAGCUCCGGAUCCACCGGUCUGCCCAA |
| | GGGAGUGGCCCUGCCACAUCGGACCGCCUGCGUGAGAUUCUCACA |
| | CGCCCGGGAUCCCAUCUUCGGAAACCAGAUCAUCCCCGACACCGC |
| | GAUUCUGUCCGUGGUGCCGUUUCACCACGGAUUCGGGAUGUUCAC |
| | CACUCUGGGUUACCUGAUUUGCGGCUUCCGCGUGGUGCUGAUGUA |
| | CCGGUUCGAGGAGGAACUGUUUCUGCGGAGCCUCCAGGAUUACAA |
| | GAUUCAGAGCGCCCUUCUGGUCCCGACUCUCUUCUCGUUCUUUGC |
| | CAAGUCCACACUUAUUGACAAAUACGAUCUCUCCAACCUCCAUGA |
| | AAUUGCCUCAGGAGGGGCUCCGCUGAGCAAGGAGGUCGGAGAGGC |
| | UGUGGCCAAGCGGUUCCACCUACCCGGCAUUAGACAGGGUUACGG |
| | CCUGACGGAAACCACCUCCGCGAUCCUGAUCACUCCGGAGGGCGA |
| | UGACAAGCCUGGAGCUGUGGGGAAGGUCGUUCCAUUCUUUGAAGC |
| | CAAGGUCGUCGACCUGGAUACCGGCAAAACCCUGGGUGUCAACCA |
| | GCGCGGAGAGCUGUGUGUGCGCGGACCUAUGAUUAUGUCCGGCUA |
| | CGUGAACAACCCCGAGGCCACUAACGCCCUCAUCGACAAGGACGG |
| | CUGGCUGCACUCCGGGGACAUCGCCUACUGGGACGAGGACGAGCA |
| | CUUCUUCAUUGUGGACCGGCUGAAGUCCCUUAUCAAGUAUAAGGG |
| | CUACCAAGUCGCACCGGCUGAACUCGAAUCAAUCUUGCUGCAACA |
| | CCCCAACAUCUUCGACGCCGGAGUGGCAGGACUGCCUGAUGACGA |
| | CGCGGGAGAACUCCCAGCAGCCGUGGUGGUGCUGGAACAUGGCAA |
| | AACCAUGACCGAGAAGGAAAUCGUGGAUUACGUGGCGUCGCAAGU |
| | CACCACUGCCAAGAAGCUGAGGGGGGGAGUGGUCUUUGUGGAUGA |
| | AGUGCCGAAGGGGCUGACGGGGAAGCUGGACGCCAGGAAGAUUAG |
| | GGAGAUCCUGAUUAAGGCCAAGAAGGGCGGCAAAAUUGCCGUGUA |
| | AUAGUGAGAUCUGGAAACAGCUAUGACCAUGGAUAACGUCGNNN |
| | NNNNNNNGUCGUGACUGGGAAAACCAGACUAGUGACUGACUAGG |
| | AUCUGGUUACCACUAAACCAGCCUCAAGAACACCCGAAUGGAGUC |
| | UCUAAGCUACAUAAUACCAACUUACACUUACAAAAUGUUGUCCCC |
| | CAAAAUGUAGCCAUUCGUAUCUGCUCCUAAUAAAAAGAAAGUUUC |
| | UUCACAUGCGGCCGCAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAA (SEQ ID NO.: 14) |
| mRNA4 | GGGAAAUAAGAGAGAAAAGAAGAGUAAGAAGAAAUAUAAGACCC |
| | CGGCGCCGCCACCAUGGAGGACGCCAAGAACAUCAAGAAGGGCCC |
| | UGCGCCAUUCUACCCCCUGGAAGAUGGAACUGCCGGCGAACAGUU |
| | GCACAAGGCCAUGAAGCGCUACGCCUUGGUGCCCGGUACUAUCGC |
| | CUUCACCGACGCCCACAUCGAAGUGGACAUAACCUACGCGGAGUA |
| | CUUCGAAAUGUCCGUGAGACUGGCCGAGGCUAUGAAGAGAUACGG |
| | ACUGAACACCAACCACCGCAUCGUGGUCUGCUCGGAGAACAGCCU |
| | GCAGUUCUUCAUGCCUGUCCUCGGUGCAUUGUUCAUCGGAGUGGC |
| | GGUGGCUCCUGCAAACGACAUCUACAACGAACGCGAACUCCUGAA |
| | CUCCAUGGGCAUCAGCCAGCCGACUGUAGUGUUCGUGAGCAAAAA |
| | GGGACUCCAGAAGAUCCUGAACGUGCAGAAGAAGCUGCCGAUCAU |
| | UCAAAAGAUUAUCAUCAUGGACUCCAAGACCGACUACCAGGGCUU |
| | CCAGUCGAUGUAUACCUUCGUGACCUCCCAUCUUCCGCCGGGGUU |
| | CAAUGAGUACGAUUUCGUGCCUGAGUCUUUCGACCGGGACAAGAC |
| | UAUCGCCCUCAUCAUGAAUAGCUCCGGAUCCACCGGUCUGCCCAA |
| | GGGAGUGGCCCUGCCACAUCGGACCGCCUGCGUGAGAUUCUCACA |
| | CGCCCGGGAUCCCAUCUUCGGAAACCAGAUCAUCCCCGACACCGC |
| | GAUUCUGUCCGUGGUGCCGUUUCACCACGGAUUCGGGAUGUUCAC |
| | CACUCUGGGUUACCUGAUUUGCGGCUUCCGCGUGGUGCUGAUGUA |
| | CCGGUUCGAGGAGGAACUGUUUCUGCGGAGCCUCCAGGAUUACAA |
| | GAUUCAGAGCGCCCUUCUGGUCCCGACUCUCUUCUCGUUCUUUGC |
| | CAAGUCCACACUUAUUGACAAAUACGAUCUCUCCAACCUCCAUGA |
| | AAUUGCCUCAGGAGGGGCUCCGCUGAGCAAGGAGGUCGGAGAGGC |
| | UGUGGCCAAGCGGUUCCACCUACCCGGCAUUAGACAGGGUUACGG |
| | CCUGACGGAAACCACCUCCGCGAUCCUGAUCACUCCGGAGGGCGA |
| | UGACAAGCCUGGAGCUGUGGGGAAGGUCGUUCCAUUCUUUGAAGC |
| | CAAGGUCGUCGACCUGGAUACCGGCAAAACCCUGGGUGUCAACCA |
| | GCGCGGAGAGCUGUGUGUGCGCGGACCUAUGAUUAUGUCCGGCUA |
| | CGUGAACAACCCCGAGGCCACUAACGCCCUCAUCGACAAGGACGG |
| | CUGGCUGCACUCCGGGGACAUCGCCUACUGGGACGAGGACGAGCA |
| | CUUCUUCAUUGUGGACCGGCUGAAGUCCCUUAUCAAGUAUAAGGG |
| | CUACCAAGUCGCACCGGCUGAACUCGAAUCAAUCUUGCUGCAACA |
| | CCCCAACAUCUUCGACGCCGGAGUGGCAGGACUGCCUGAUGACGA |
| | CGCGGGAGAACUCCCAGCAGCCGUGGUGGUGCUGGAACAUGGCAA |
| | AACCAUGACCGAGAAGGAAAUCGUGGAUUACGUGGCGUCGCAAGU |
| | CACCACUGCCAAGAAGCUGAGGGGGGGAGUGGUCUUUGUGGAUGA |
| | AGUGCCGAAGGGGCUGACGGGGAAGCUGGACGCCAGGAAGAUUAG |
| | GGAGAUCCUGAUUAAGGCCAAGAAGGGCGGCAAAAUUGCCGUGUA |
| | AUAGUGAGAUCUGGAAACAGCUAUGACCAUGGAAAGCAUCGNNN |
| | NNNNNNNGUCGUGACUGGGAAAACCAGACUAGUGACUGACUAGG |
| | AUCUGGUUACCACUAAACCAGCCUCAAGAACACCCGAAUGGAGUC |
| | UCUAAGCUACAUAAUACCAACUUACACUUACAAAAUGUUGUCCCC |
| | CAAAAUGUAGCCAUUCGUAUCUGCUCCUAAUAAAAAGAAAGUUUC |
| | UUCACAUGCGGCCGCAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAA (SEQ ID NO.: 15) |
| mRNA5 | GGGAAAUAAGAGAGAAAAGAAGAGUAAGAAGAAAUAUAAGACCC |
| | CGGCGCCGCCACCAUGGAGGACGCCAAGAACAUCAAGAAGGGCCC |
| | UGCGCCAUUCUACCCCCUGGAAGAUGGAACUGCCGGCGAACAGUU |
| | GCACAAGGCCAUGAAGCGCUACGCCUUGGUGCCCGGUACUAUCGC |
| | CUUCACCGACGCCCACAUCGAAGUGGACAUAACCUACGCGGAGUA |
| | CUUCGAAAUGUCCGUGAGACUGGCCGAGGCUAUGAAGAGAUACGG |
| | ACUGAACACCAACCACCGCAUCGUGGUCUGCUCGGAGAACAGCCU |
| | GCAGUUCUUCAUGCCUGUCCUCGGUGCAUUGUUCAUCGGAGUGGC |
| | GGUGGCUCCUGCAAACGACAUCUACAACGAACGCGAACUCCUGAA |
| | CUCCAUGGGCAUCAGCCAGCCGACUGUAGUGUUCGUGAGCAAAAA |
| | GGGACUCCAGAAGAUCCUGAACGUGCAGAAGAAGCUGCCGAUCAU |
| | UCAAAAGAUUAUCAUCAUGGACUCCAAGACCGACUACCAGGGCUU |
| | CCAGUCGAUGUAUACCUUCGUGACCUCCCAUCUUCCGCCGGGGUU |
| | CAAUGAGUACGAUUUCGUGCCUGAGUCUUUCGACCGGGACAAGAC |
| | UAUCGCCCUCAUCAUGAAUAGCUCCGGAUCCACCGGUCUGCCCAA |
| | GGGAGUGGCCCUGCCACAUCGGACCGCCUGCGUGAGAUUCUCACA |
| | CGCCCGGGAUCCCAUCUUCGGAAACCAGAUCAUCCCCGACACCGC |
| | GAUUCUGUCCGUGGUGCCGUUUCACCACGGAUUCGGGAUGUUCAC |
| | CACUCUGGGUUACCUGAUUUGCGGCUUCCGCGUGGUGCUGAUGUA |
| | CCGGUUCGAGGAGGAACUGUUUCUGCGGAGCCUCCAGGAUUACAA |
| | GAUUCAGAGCGCCCUUCUGGUCCCGACUCUCUUCUCGUUCUUUGC |
| | CAAGUCCACACUUAUUGACAAAUACGAUCUCUCCAACCUCCAUGA |
| | AAUUGCCUCAGGAGGGGCUCCGCUGAGCAAGGAGGUCGGAGAGGC |
| | UGUGGCCAAGCGGUUCCACCUACCCGGCAUUAGACAGGGUUACGG |
| | CCUGACGGAAACCACCUCCGCGAUCCUGAUCACUCCGGAGGGCGA |
| | UGACAAGCCUGGAGCUGUGGGGAAGGUCGUUCCAUUCUUUGAAGC |
| | CAAGGUCGUCGACCUGGAUACCGGCAAAACCCUGGGUGUCAACCA |
| | GCGCGGAGAGCUGUGUGUGCGCGGACCUAUGAUUAUGUCCGGCUA |
| | CGUGAACAACCCCGAGGCCACUAACGCCCUCAUCGACAAGGACGG |
| | CUGGCUGCACUCCGGGGACAUCGCCUACUGGGACGAGGACGAGCA |
| | CUUCUUCAUUGUGGACCGGCUGAAGUCCCUUAUCAAGUAUAAGGG |
| | CUACCAAGUCGCACCGGCUGAACUCGAAUCAAUCUUGCUGCAACA |
| | CCCCAACAUCUUCGACGCCGGAGUGGCAGGACUGCCUGAUGACGA |
| | CGCGGGAGAACUCCCAGCAGCCGUGGUGGUGCUGGAACAUGGCAA |
| | AACCAUGACCGAGAAGGAAAUCGUGGAUUACGUGGCGUCGCAAGU |
| | CACCACUGCCAAGAAGCUGAGGGGGGGAGUGGUCUUUGUGGAUGA |
| | AGUGCCGAAGGGGCUGACGGGGAAGCUGGACGCCAGGAAGAUUAG |
| | GGAGAUCCUGAUUAAGGCCAAGAAGGGCGGCAAAAUUGCCGUGUA |
| | AUAGUGAGAUCUGGAAACAGCUAUGACCAUGGAGCAAUUCCNNNN |
| | NNNNNNGUCGUGACUGGGAAAACCAGACUAGUGACUGACUAGGA |
| | UCUGGUUACCACUAAACCAGCCUCAAGAACACCCGAAUGGAGUCU |
| | CUAAGCUACAUAAUACCAACUUACACUUACAAAAUGUUGUCCCCC |
| | AAAAUGUAGCCAUUCGUAUCUGCUCCUAAUAAAAAGAAAGUUUCU |
| | UCACAUGCGGCCGCAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAA (SEQ ID NO.: 16) |
| mRNA6 | GGGAAAUAAGAGAGAAAAGAAGAGUAAGAAGAAAUAUAAGACCC |
| | CGGCGCCGCCACCAUGGAGGACGCCAAGAACAUCAAGAAGGGCCC |
| | UGCGCCAUUCUACCCCCUGGAAGAUGGAACUGCCGGCGAACAGUU |
| | GCACAAGGCCAUGAAGCGCUACGCCUUGGUGCCCGGUACUAUCGC |
| | CUUCACCGACGCCCACAUCGAAGUGGACAUAACCUACGCGGAGUA |
| | CUUCGAAAUGUCCGUGAGACUGGCCGAGGCUAUGAAGAGAUACGG |
| | ACUGAACACCAACCACCGCAUCGUGGUCUGCUCGGAGAACAGCCU |
| | GCAGUUCUUCAUGCCUGUCCUCGGUGCAUUGUUCAUCGGAGUGGC |
| | GGUGGCUCCUGCAAACGACAUCUACAACGAACGCGAACUCCUGAA |
| | CUCCAUGGGCAUCAGCCAGCCGACUGUAGUGUUCGUGAGCAAAAA |
| | GGGACUCCAGAAGAUCCUGAACGUGCAGAAGAAGCUGCCGAUCAU |
| | UCAAAAGAUUAUCAUCAUGGACUCCAAGACCGACUACCAGGGCUU |
| | CCAGUCGAUGUAUACCUUCGUGACCUCCCAUCUUCCGCCGGGGUU |
| | CAAUGAGUACGAUUUCGUGCCUGAGUCUUUCGACCGGGACAAGAC |
| | UAUCGCCCUCAUCAUGAAUAGCUCCGGAUCCACCGGUCUGCCCAA |
| | GGGAGUGGCCCUGCCACAUCGGACCGCCUGCGUGAGAUUCUCACA |
| | CGCCCGGGAUCCCAUCUUCGGAAACCAGAUCAUCCCCGACACCGC |
| | GAUUCUGUCCGUGGUGCCGUUUCACCACGGAUUCGGGAUGUUCAC |
| | CACUCUGGGUUACCUGAUUUGCGGCUUCCGCGUGGUGCUGAUGUA |
| | CCGGUUCGAGGAGGAACUGUUUCUGCGGAGCCUCCAGGAUUACAA |
| | GAUUCAGAGCGCCCUUCUGGUCCCGACUCUCUUCUCGUUCUUUGC |
| | CAAGUCCACACUUAUUGACAAAUACGAUCUCUCCAACCUCCAUGA |
| | AAUUGCCUCAGGAGGGGCUCCGCUGAGCAAGGAGGUCGGAGAGGC |
| | UGUGGCCAAGCGGUUCCACCUACCCGGCAUUAGACAGGGUUACGG |
| | CCUGACGGAAACCACCUCCGCGAUCCUGAUCACUCCGGAGGGCGA |
| | UGACAAGCCUGGAGCUGUGGGGAAGGUCGUUCCAUUCUUUGAAGC |
| | CAAGGUCGUCGACCUGGAUACCGGCAAAACCCUGGGUGUCAACCA |
| | GCGCGGAGAGCUGUGUGUGCGCGGACCUAUGAUUAUGUCCGGCUA |
| | CGUGAACAACCCCGAGGCCACUAACGCCCUCAUCGACAAGGACGG |
| | CUGGCUGCACUCCGGGGACAUCGCCUACUGGGACGAGGACGAGCA |
| | CUUCUUCAUUGUGGACCGGCUGAAGUCCCUUAUCAAGUAUAAGGG |
| | CUACCAAGUCGCACCGGCUGAACUCGAAUCAAUCUUGCUGCAACA |
| | CCCCAACAUCUUCGACGCCGGAGUGGCAGGACUGCCUGAUGACGA |
| | CGCGGGAGAACUCCCAGCAGCCGUGGUGGUGCUGGAACAUGGCAA |
| | AACCAUGACCGAGAAGGAAAUCGUGGAUUACGUGGCGUCGCAAGU |
| | CACCACUGCCAAGAAGCUGAGGGGGGGAGUGGUCUUUGUGGAUGA |
| | AGUGCCGAAGGGGCUGACGGGGAAGCUGGACGCCAGGAAGAUUAG |
| | GGAGAUCCUGAUUAAGGCCAAGAAGGGCGGCAAAAUUGCCGUGUA |
| | AUAGUGAGAUCUGGAAACAGCUAUGACCAUGGAGCUACAACNNNN |
| | NNNNNNGUCGUGACUGGGAAAACCAGACUAGUGACUGACUAGGA |
| | UCUGGUUACCACUAAACCAGCCUCAAGAACACCCGAAUGGAGUCU |
| | CUAAGCUACAUAAUACCAACUUACACUUACAAAAUGUUGUCCCCC |
| | AAAAUGUAGCCAUUCGUAUCUGCUCCUAAUAAAAAGAAAGUUUCU |
| | UCACAUGCGGCCGCAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAA (SEQ ID NO.: 17) |
| mRNA7 | GGGAAAUAAGAGAGAAAAGAAGAGUAAGAAGAAAUAUAAGACCC |
| | CGGCGCCGCCACCAUGGAGGACGCCAAGAACAUCAAGAAGGGCCC |
| | UGCGCCAUUCUACCCCCUGGAAGAUGGAACUGCCGGCGAACAGUU |
| | GCACAAGGCCAUGAAGCGCUACGCCUUGGUGCCCGGUACUAUCGC |
| | CUUCACCGACGCCCACAUCGAAGUGGACAUAACCUACGCGGAGUA |
| | CUUCGAAAUGUCCGUGAGACUGGCCGAGGCUAUGAAGAGAUACGG |
| | ACUGAACACCAACCACCGCAUCGUGGUCUGCUCGGAGAACAGCCU |
| | GCAGUUCUUCAUGCCUGUCCUCGGUGCAUUGUUCAUCGGAGUGGC |
| | GGUGGCUCCUGCAAACGACAUCUACAACGAACGCGAACUCCUGAA |
| | CUCCAUGGGCAUCAGCCAGCCGACUGUAGUGUUCGUGAGCAAAAA |
| | GGGACUCCAGAAGAUCCUGAACGUGCAGAAGAAGCUGCCGAUCAU |
| | UCAAAAGAUUAUCAUCAUGGACUCCAAGACCGACUACCAGGGCUU |
| | CCAGUCGAUGUAUACCUUCGUGACCUCCCAUCUUCCGCCGGGGUU |
| | CAAUGAGUACGAUUUCGUGCCUGAGUCUUUCGACCGGGACAAGAC |
| | UAUCGCCCUCAUCAUGAAUAGCUCCGGAUCCACCGGUCUGCCCAA |
| | GGGAGUGGCCCUGCCACAUCGGACCGCCUGCGUGAGAUUCUCACA |
| | CGCCCGGGAUCCCAUCUUCGGAAACCAGAUCAUCCCCGACACCGC |
| | GAUUCUGUCCGUGGUGCCGUUUCACCACGGAUUCGGGAUGUUCAC |
| | CACUCUGGGUUACCUGAUUUGCGGCUUCCGCGUGGUGCUGAUGUA |
| | CCGGUUCGAGGAGGAACUGUUUCUGCGGAGCCUCCAGGAUUACAA |
| | GAUUCAGAGCGCCCUUCUGGUCCCGACUCUCUUCUCGUUCUUUGC |
| | CAAGUCCACACUUAUUGACAAAUACGAUCUCUCCAACCUCCAUGA |
| | AAUUGCCUCAGGAGGGGCUCCGCUGAGCAAGGAGGUCGGAGAGGC |
| | UGUGGCCAAGCGGUUCCACCUACCCGGCAUUAGACAGGGUUACGG |
| | CCUGACGGAAACCACCUCCGCGAUCCUGAUCACUCCGGAGGGCGA |
| | UGACAAGCCUGGAGCUGUGGGGAAGGUCGUUCCAUUCUUUGAAGC |
| | CAAGGUCGUCGACCUGGAUACCGGCAAAACCCUGGGUGUCAACCA |
| | GCGCGGAGAGCUGUGUGUGCGCGGACCUAUGAUUAUGUCCGGCUA |
| | CGUGAACAACCCCGAGGCCACUAACGCCCUCAUCGACAAGGACGG |
| | CUGGCUGCACUCCGGGGACAUCGCCUACUGGGACGAGGACGAGCA |
| | CUUCUUCAUUGUGGACCGGCUGAAGUCCCUUAUCAAGUAUAAGGG |
| | CUACCAAGUCGCACCGGCUGAACUCGAAUCAAUCUUGCUGCAACA |
| | CCCCAACAUCUUCGACGCCGGAGUGGCAGGACUGCCUGAUGACGA |
| | CGCGGGAGAACUCCCAGCAGCCGUGGUGGUGCUGGAACAUGGCAA |
| | AACCAUGACCGAGAAGGAAAUCGUGGAUUACGUGGCGUCGCAAGU |
| | CACCACUGCCAAGAAGCUGAGGGGGGGAGUGGUCUUUGUGGAUGA |
| | AGUGCCGAAGGGGCUGACGGGGAAGCUGGACGCCAGGAAGAUUAG |
| | GGAGAUCCUGAUUAAGGCCAAGAAGGGCGGCAAAAUUGCCGUGUA |
| | AUAGUGAGAUCUGGAAACAGCUAUGACCAUGGAUAGGAGCUNNN |
| | NNNNNNNGUCGUGACUGGGAAAACCAGACUAGUGACUGACUAGG |
| | AUCUGGUUACCACUAAACCAGCCUCAAGAACACCCGAAUGGAGUC |
| | UCUAAGCUACAUAAUACCAACUUACACUUACAAAAUGUUGUCCCC |
| | CAAAAUGUAGCCAUUCGUAUCUGCUCCUAAUAAAAAGAAAGUUUC |
| | UUCACAUGCGGCCGCAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAA (SEQ ID NO.: 18) |
| mRNA8 | GGGAAAUAAGAGAGAAAAGAAGAGUAAGAAGAAAUAUAAGACCC |
| | CGGCGCCGCCACCAUGGAGGACGCCAAGAACAUCAAGAAGGGCCC |
| | UGCGCCAUUCUACCCCCUGGAAGAUGGAACUGCCGGCGAACAGUU |
| | GCACAAGGCCAUGAAGCGCUACGCCUUGGUGCCCGGUACUAUCGC |
| | CUUCACCGACGCCCACAUCGAAGUGGACAUAACCUACGCGGAGUA |
| | CUUCGAAAUGUCCGUGAGACUGGCCGAGGCUAUGAAGAGAUACGG |
| | ACUGAACACCAACCACCGCAUCGUGGUCUGCUCGGAGAACAGCCU |
| | GCAGUUCUUCAUGCCUGUCCUCGGUGCAUUGUUCAUCGGAGUGGC |
| | GGUGGCUCCUGCAAACGACAUCUACAACGAACGCGAACUCCUGAA |
| | CUCCAUGGGCAUCAGCCAGCCGACUGUAGUGUUCGUGAGCAAAAA |
| | GGGACUCCAGAAGAUCCUGAACGUGCAGAAGAAGCUGCCGAUCAU |
| | UCAAAAGAUUAUCAUCAUGGACUCCAAGACCGACUACCAGGGCUU |
| | CCAGUCGAUGUAUACCUUCGUGACCUCCCAUCUUCCGCCGGGGUU |
| | CAAUGAGUACGAUUUCGUGCCUGAGUCUUUCGACCGGGACAAGAC |
| | UAUCGCCCUCAUCAUGAAUAGCUCCGGAUCCACCGGUCUGCCCAA |
| | GGGAGUGGCCCUGCCACAUCGGACCGCCUGCGUGAGAUUCUCACA |
| | CGCCCGGGAUCCCAUCUUCGGAAACCAGAUCAUCCCCGACACCGC |
| | GAUUCUGUCCGUGGUGCCGUUUCACCACGGAUUCGGGAUGUUCAC |
| | CACUCUGGGUUACCUGAUUUGCGGCUUCCGCGUGGUGCUGAUGUA |
| | CCGGUUCGAGGAGGAACUGUUUCUGCGGAGCCUCCAGGAUUACAA |
| | GAUUCAGAGCGCCCUUCUGGUCCCGACUCUCUUCUCGUUCUUUGC |
| | CAAGUCCACACUUAUUGACAAAUACGAUCUCUCCAACCUCCAUGA |
| | AAUUGCCUCAGGAGGGGCUCCGCUGAGCAAGGAGGUCGGAGAGGC |
| | UGUGGCCAAGCGGUUCCACCUACCCGGCAUUAGACAGGGUUACGG |
| | CCUGACGGAAACCACCUCCGCGAUCCUGAUCACUCCGGAGGGCGA |
| | UGACAAGCCUGGAGCUGUGGGGAAGGUCGUUCCAUUCUUUGAAGC |
| | CAAGGUCGUCGACCUGGAUACCGGCAAAACCCUGGGUGUCAACCA |
| | GCGCGGAGAGCUGUGUGUGCGCGGACCUAUGAUUAUGUCCGGCUA |
| | CGUGAACAACCCCGAGGCCACUAACGCCCUCAUCGACAAGGACGG |
| | CUGGCUGCACUCCGGGGACAUCGCCUACUGGGACGAGGACGAGCA |
| | CUUCUUCAUUGUGGACCGGCUGAAGUCCCUUAUCAAGUAUAAGGG |
| | CUACCAAGUCGCACCGGCUGAACUCGAAUCAAUCUUGCUGCAACA |
| | CCCCAACAUCUUCGACGCCGGAGUGGCAGGACUGCCUGAUGACGA |
| | CGCGGGAGAACUCCCAGCAGCCGUGGUGGUGCUGGAACAUGGCAA |
| | AACCAUGACCGAGAAGGAAAUCGUGGAUUACGUGGCGUCGCAAGU |
| | CACCACUGCCAAGAAGCUGAGGGGGGGAGUGGUCUUUGUGGAUGA |
| | AGUGCCGAAGGGGCUGACGGGGAAGCUGGACGCCAGGAAGAUUAG |
| | GGAGAUCCUGAUUAAGGCCAAGAAGGGCGGCAAAAUUGCCGUGUA |
| | AUAGUGAGAUCUGGAAACAGCUAUGACCAUGGAUGGAUGGUNNN |
| | NNNNNNNGUCGUGACUGGGAAAACCAGACUAGUGACUGACUAGG |
| | AUCUGGUUACCACUAAACCAGCCUCAAGAACACCCGAAUGGAGUC |
| | UCUAAGCUACAUAAUACCAACUUACACUUACAAAAUGUUGUCCCC |
| | CAAAAUGUAGCCAUUCGUAUCUGCUCCUAAUAAAAAGAAAGUUUC |
| | UUCACAUGCGGCCGCAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAA (SEQ ID NO.: 19) |
| mRNA9 | GGGAAAUAAGAGAGAAAAGAAGAGUAAGAAGAAAUAUAAGACCC |
| | CGGCGCCGCCACCAUGGAGGACGCCAAGAACAUCAAGAAGGGCCC |
| | UGCGCCAUUCUACCCCCUGGAAGAUGGAACUGCCGGCGAACAGUU |
| | GCACAAGGCCAUGAAGCGCUACGCCUUGGUGCCCGGUACUAUCGC |
| | CUUCACCGACGCCCACAUCGAAGUGGACAUAACCUACGCGGAGUA |
| | CUUCGAAAUGUCCGUGAGACUGGCCGAGGCUAUGAAGAGAUACGG |
| | ACUGAACACCAACCACCGCAUCGUGGUCUGCUCGGAGAACAGCCU |
| | GCAGUUCUUCAUGCCUGUCCUCGGUGCAUUGUUCAUCGGAGUGGC |
| | GGUGGCUCCUGCAAACGACAUCUACAACGAACGCGAACUCCUGAA |
| | CUCCAUGGGCAUCAGCCAGCCGACUGUAGUGUUCGUGAGCAAAAA |
| | GGGACUCCAGAAGAUCCUGAACGUGCAGAAGAAGCUGCCGAUCAU |
| | UCAAAAGAUUAUCAUCAUGGACUCCAAGACCGACUACCAGGGCUU |
| | CCAGUCGAUGUAUACCUUCGUGACCUCCCAUCUUCCGCCGGGGUU |
| | CAAUGAGUACGAUUUCGUGCCUGAGUCUUUCGACCGGGACAAGAC |
| | UAUCGCCCUCAUCAUGAAUAGCUCCGGAUCCACCGGUCUGCCCAA |
| | GGGAGUGGCCCUGCCACAUCGGACCGCCUGCGUGAGAUUCUCACA |
| | CGCCCGGGAUCCCAUCUUCGGAAACCAGAUCAUCCCCGACACCGC |
| | GAUUCUGUCCGUGGUGCCGUUUCACCACGGAUUCGGGAUGUUCAC |
| | CACUCUGGGUUACCUGAUUUGCGGCUUCCGCGUGGUGCUGAUGUA |
| | CCGGUUCGAGGAGGAACUGUUUCUGCGGAGCCUCCAGGAUUACAA |
| | GAUUCAGAGCGCCCUUCUGGUCCCGACUCUCUUCUCGUUCUUUGC |
| | CAAGUCCACACUUAUUGACAAAUACGAUCUCUCCAACCUCCAUGA |
| | AAUUGCCUCAGGAGGGGCUCCGCUGAGCAAGGAGGUCGGAGAGGC |
| | UGUGGCCAAGCGGUUCCACCUACCCGGCAUUAGACAGGGUUACGG |
| | CCUGACGGAAACCACCUCCGCGAUCCUGAUCACUCCGGAGGGCGA |
| | UGACAAGCCUGGAGCUGUGGGGAAGGUCGUUCCAUUCUUUGAAGC |
| | CAAGGUCGUCGACCUGGAUACCGGCAAAACCCUGGGUGUCAACCA |
| | GCGCGGAGAGCUGUGUGUGCGCGGACCUAUGAUUAUGUCCGGCUA |
| | CGUGAACAACCCCGAGGCCACUAACGCCCUCAUCGACAAGGACGG |
| | CUGGCUGCACUCCGGGGACAUCGCCUACUGGGACGAGGACGAGCA |
| | CUUCUUCAUUGUGGACCGGCUGAAGUCCCUUAUCAAGUAUAAGGG |
| | CUACCAAGUCGCACCGGCUGAACUCGAAUCAAUCUUGCUGCAACA |
| | CCCCAACAUCUUCGACGCCGGAGUGGCAGGACUGCCUGAUGACGA |
| | CGCGGGAGAACUCCCAGCAGCCGUGGUGGUGCUGGAACAUGGCAA |
| | AACCAUGACCGAGAAGGAAAUCGUGGAUUACGUGGCGUCGCAAGU |
| | CACCACUGCCAAGAAGCUGAGGGGGGGAGUGGUCUUUGUGGAUGA |
| | AGUGCCGAAGGGGCUGACGGGGAAGCUGGACGCCAGGAAGAUUAG |
| | GGAGAUCCUGAUUAAGGCCAAGAAGGGCGGCAAAAUUGCCGUGUA |
| | AUAGUGAGAUCUGGAAACAGCUAUGACCAUGGAUACUAGCGNNN |
| | NNNNNNNGUCGUGACUGGGAAAACCAGACUAGUGACUGACUAGG |
| | AUCUGGUUACCACUAAACCAGCCUCAAGAACACCCGAAUGGAGUC |
| | UCUAAGCUACAUAAUACCAACUUACACUUACAAAAUGUUGUCCCC |
| | CAAAAUGUAGCCAUUCGUAUCUGCUCCUAAUAAAAAGAAAGUUUC |
| | UUCACAUGCGGCCGCAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAA (SEQ ID NO.: 20) |
| mRNA10 | GGGAAAUAAGAGAGAAAAGAAGAGUAAGAAGAAAUAUAAGACCC |
| | CGGCGCCGCCACCAUGGAGGACGCCAAGAACAUCAAGAAGGGCCC |
| | UGCGCCAUUCUACCCCCUGGAAGAUGGAACUGCCGGCGAACAGUU |
| | GCACAAGGCCAUGAAGCGCUACGCCUUGGUGCCCGGUACUAUCGC |
| | CUUCACCGACGCCCACAUCGAAGUGGACAUAACCUACGCGGAGUA |
| | CUUCGAAAUGUCCGUGAGACUGGCCGAGGCUAUGAAGAGAUACGG |
| | ACUGAACACCAACCACCGCAUCGUGGUCUGCUCGGAGAACAGCCU |
| | GCAGUUCUUCAUGCCUGUCCUCGGUGCAUUGUUCAUCGGAGUGGC |
| | GGUGGCUCCUGCAAACGACAUCUACAACGAACGCGAACUCCUGAA |
| | CUCCAUGGGCAUCAGCCAGCCGACUGUAGUGUUCGUGAGCAAAAA |
| | GGGACUCCAGAAGAUCCUGAACGUGCAGAAGAAGCUGCCGAUCAU |
| | UCAAAAGAUUAUCAUCAUGGACUCCAAGACCGACUACCAGGGCUU |
| | CCAGUCGAUGUAUACCUUCGUGACCUCCCAUCUUCCGCCGGGGUU |
| | CAAUGAGUACGAUUUCGUGCCUGAGUCUUUCGACCGGGACAAGAC |
| | UAUCGCCCUCAUCAUGAAUAGCUCCGGAUCCACCGGUCUGCCCAA |
| | GGGAGUGGCCCUGCCACAUCGGACCGCCUGCGUGAGAUUCUCACA |
| | CGCCCGGGAUCCCAUCUUCGGAAACCAGAUCAUCCCCGACACCGC |
| | GAUUCUGUCCGUGGUGCCGUUUCACCACGGAUUCGGGAUGUUCAC |
| | CACUCUGGGUUACCUGAUUUGCGGCUUCCGCGUGGUGCUGAUGUA |
| | CCGGUUCGAGGAGGAACUGUUUCUGCGGAGCCUCCAGGAUUACAA |
| | GAUUCAGAGCGCCCUUCUGGUCCCGACUCUCUUCUCGUUCUUUGC |
| | CAAGUCCACACUUAUUGACAAAUACGAUCUCUCCAACCUCCAUGA |
| | AAUUGCCUCAGGAGGGGCUCCGCUGAGCAAGGAGGUCGGAGAGGC |
| | UGUGGCCAAGCGGUUCCACCUACCCGGCAUUAGACAGGGUUACGG |
| | CCUGACGGAAACCACCUCCGCGAUCCUGAUCACUCCGGAGGGCGA |
| | UGACAAGCCUGGAGCUGUGGGGAAGGUCGUUCCAUUCUUUGAAGC |
| | CAAGGUCGUCGACCUGGAUACCGGCAAAACCCUGGGUGUCAACCA |
| | GCGCGGAGAGCUGUGUGUGCGCGGACCUAUGAUUAUGUCCGGCUA |
| | CGUGAACAACCCCGAGGCCACUAACGCCCUCAUCGACAAGGACGG |
| | CUGGCUGCACUCCGGGGACAUCGCCUACUGGGACGAGGACGAGCA |
| | CUUCUUCAUUGUGGACCGGCUGAAGUCCCUUAUCAAGUAUAAGGG |
| | CUACCAAGUCGCACCGGCUGAACUCGAAUCAAUCUUGCUGCAACA |
| | CCCCAACAUCUUCGACGCCGGAGUGGCAGGACUGCCUGAUGACGA |
| | CGCGGGAGAACUCCCAGCAGCCGUGGUGGUGCUGGAACAUGGCAA |
| | AACCAUGACCGAGAAGGAAAUCGUGGAUUACGUGGCGUCGCAAGU |
| | CACCACUGCCAAGAAGCUGAGGGGGGGAGUGGUCUUUGUGGAUGA |
| | AGUGCCGAAGGGGCUGACGGGGAAGCUGGACGCCAGGAAGAUUAG |
| | GGAGAUCCUGAUUAAGGCCAAGAAGGGCGGCAAAAUUGCCGUGUA |
| | AUAGUGAGAUCUGGAAACAGCUAUGACCAUGGACAGUGCUUNNN |
| | NNNNNNNGUCGUGACUGGGAAAACCAGACUAGUGACUGACUAGG |
| | AUCUGGUUACCACUAAACCAGCCUCAAGAACACCCGAAUGGAGUC |
| | UCUAAGCUACAUAAUACCAACUUACACUUACAAAAUGUUGUCCCC |
| | CAAAAUGUAGCCAUUCGUAUCUGCUCCUAAUAAAAAGAAAGUUUC |
| | UUCACAUGCGGCCGCAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAA |
| | AAAAAAAAAAAAA (SEQ ID NO.: 21) |
| ORNA1A | AAAAUCCGUGGCUCGCAGCAAACACCAAACGGCUCAAUAAAACAA |
| | ACACAAACCAACAAAAAGUGGCCACGCCCGGGCCACCGAUACUUC |
| | CCUUCACUCCUUCGGGACUGUUGGGGAGGAACACAACAGGGCUCC |
| | CCUGUUUUCCCAUUCCUUCCCCCUUUUCCCAACCCCAACCGCCGUA |
| | UCUGGUGGCGGCAAGACACACGGGUCUUUCCCUCUAAAGCACAAU |
| | UGUGUGUGUGUCCCAGGUCCUCCUGCGUACGGUGCGGGAGUGCUC |
| | CCACCCAACUGUUGUAAGCCUGUCCAACGCGUCGUCCUGGCAAGA |
| | CUAUGACGUCGCAUGUUCCGCUGCGGAUGCCGACCGGGUAACCGG |
| | UUCCCCAGUGUGUGUAGUGCGAUCUUCCAGGUCCUCCUGGUUGGC |
| | GUUGUCCAGAAACUGCUUCAGGUAAGUGGGGUGUGCCCAAUCCCU |
| | ACAAAGGUUGAUUCUUUCACCACCUUAGGAAUGCUCCGGAGGUAC |
| | CCCAGCAACAGCUGGGAUCUGACCGGAGGCUAAUUGUCUACGGGU |
| | GGUGUUUCCUUUUUCUUUUCACACAACUGUCACGAUGGAAGAUGC |
| | CAAGAACAUCAAGAAGGGCCCUGCUCCAUUCUACCCUCUGGAAGA |
| | UGGAACAGCCGGCGAGCAGCUGCACAAGGCCAUGAAGAGAUAUGC |
| | CCUGGUGCCUGGCACAAUCGCCUUCACAGAUGCCCACAUCGAGGU |
| | GGACAUCACCUACGCCGAGUACUUCGAGAUGUCUGUGCGGCUGGC |
| | CGAAGCUAUGAAGCGCUACGGCCUGAACACCAACCACCGGAUCGU |
| | CGUGUGCAGCGAGAACAGCCUGCAGUUCUUCAUGCCUGUGCUGGG |
| | CGCCCUGUUCAUCGGAGUUGCUGUGGCCCCUGCCAACGACAUCUA |
| | CAACGAGCGCGAGCUGCUGAACAGCAUGGGCAUCUCUCAGCCCAC |
| | CGUGGUGUUCGUGUCCAAGAAGGGACUGCAGAAAAUCCUGAACGU |
| | GCAGAAGAAGCUGCCCAUCAUCCAGAAAAUCAUCAUCAUGGACAG |
| | CAAGACCGACUACCAGGGCUUCCAGAGCAUGUACACCUUCGUGAC |
| | CAGCCAUCUGCCACCUGGCUUCAACGAGUACGACUUCGUGCCCGA |
| | GAGCUUCGACCGGGACAAGACAAUCGCCCUGAUCAUGAACAGCAG |
| | CGGCUCUACCGGACUGCCCAAAGGUGUUGCCCUGCCUCAUAGAAC |
| | UGCCUGCGUGCGGUUUAGCCACGCCAGAGAUCCCAUCUUCGGCAA |
| | CCAGAUCAUCCCCGACACAGCCAUCCUGAGCGUGGUGCCUUUUCA |
| | CCACGGCUUCGGCAUGUUCACCACACUGGGCUACCUGAUCUGCGG |
| | CUUCAGAGUGGUGCUGAUGUACCGCUUCGAGGAAGAACUGUUCCU |
| | GCGGAGCCUGCAGGACUACAAGAUCCAGUCUGCUCUGCUGGUGCC |
| | UACUCUGUUCAGCUUCUUUGCCAAGAGCACCCUGAUCGAUAAGUA |
| | CGACCUGAGCAACCUGCACGAGAUCGCCUCUGGCGGAGCCCCUCU |
| | GUCUAAAGAAGUGGGAGAAGCCGUCGCCAAGCGGUUCCAUCUGCC |
| | UGGCAUCAGACAAGGCUAUGGCCUGACCGAGACAACCAGCGCCAU |
| | UCUGAUUACCCCUGAGGGCGACGAUAAGCCUGGCGCUGUGGGAAA |
| | AGUGGUGCCAUUCUUCGAGGCCAAGGUGGUGGAUCUGGACACCGG |
| | CAAAACACUGGGCGUUAACCAGAGGGGCGAGCUGUGUGUUAGAGG |
| | CCCUAUGAUCAUGAGCGGCUACGUGAACAACCCCGAGGCCACAAA |
| | CGCUCUGAUCGACAAGGAUGGAUGGCUGCACAGCGGCGACAUUGC |
| | CUACUGGGACGAAGAUGAGCACUUCUUCAUCGUGGACCGGCUGAA |
| | GUCCCUGAUCAAGUACAAGGGCUACCAGGUGGCCCCAGCCGAGCU |
| | GGAAUCUAUUCUGCUGCAACACCCCAACAUCUUCGAUGCCGGCGU |
| | UGCAGGACUGCCCGAUGAUGAUGCUGGCGAACUGCCAGCUGCUGU |
| | GGUGGUGCUGGAACACGGCAAGACCAUGACCGAGAAAGAAAUCGU |
| | GGACUACGUGGCCAGCCAAGUGACCACCGCCAAGAAACUGAGAGG |
| | CGGCGUGGUGUUUGUGGACGAGGUGCCAAAAGGCCUGACAGGCAA |
| | GCUGGACGCCCGGAAGAUCAGAGAGAUCCUGAUUAAGGCCAAGAA |
| | AGGCGGCAAGAUCGCCGUGUAAUAGCUAACUAGACAAAACACCAA |
| | ACAAACAAGCUGCGAGCCACGGACUU (SEQ ID NO.: 22) |
| ORNA2A | AAAAUCCGUGGCUCGCAGCAAACACCAAACCGACCUAAAAAACAA |
| | ACACAAACCAACAAAAAGUGGCCACGCCCGGGCCACCGAUACUUC |
| | CCUUCACUCCUUCGGGACUGUUGGGGAGGAACACAACAGGGCUCC |
| | CCUGUUUUCCCAUUCCUUCCCCCUUUUCCCAACCCCAACCGCCGUA |
| | UCUGGUGGCGGCAAGACACACGGGUCUUUCCCUCUAAAGCACAAU |
| | UGUGUGUGUGUCCCAGGUCCUCCUGCGUACGGUGCGGGAGUGCUC |
| | CCACCCAACUGUUGUAAGCCUGUCCAACGCGUCGUCCUGGCAAGA |
| | CUAUGACGUCGCAUGUUCCGCUGCGGAUGCCGACCGGGUAACCGG |
| | UUCCCCAGUGUGUGUAGUGCGAUCUUCCAGGUCCUCCUGGUUGGC |
| | GUUGUCCAGAAACUGCUUCAGGUAAGUGGGGUGUGCCCAAUCCCU |
| | ACAAAGGUUGAUUCUUUCACCACCUUAGGAAUGCUCCGGAGGUAC |
| | CCCAGCAACAGCUGGGAUCUGACCGGAGGCUAAUUGUCUACGGGU |
| | GGUGUUUCCUUUUUCUUUUCACACAACUGUCACGAUGGAAGAUGC |
| | CAAGAACAUCAAGAAGGGCCCUGCUCCAUUCUACCCUCUGGAAGA |
| | UGGAACAGCCGGCGAGCAGCUGCACAAGGCCAUGAAGAGAUAUGC |
| | CCUGGUGCCUGGCACAAUCGCCUUCACAGAUGCCCACAUCGAGGU |
| | GGACAUCACCUACGCCGAGUACUUCGAGAUGUCUGUGCGGCUGGC |
| | CGAAGCUAUGAAGCGCUACGGCCUGAACACCAACCACCGGAUCGU |
| | CGUGUGCAGCGAGAACAGCCUGCAGUUCUUCAUGCCUGUGCUGGG |
| | CGCCCUGUUCAUCGGAGUUGCUGUGGCCCCUGCCAACGACAUCUA |
| | CAACGAGCGCGAGCUGCUGAACAGCAUGGGCAUCUCUCAGCCCAC |
| | CGUGGUGUUCGUGUCCAAGAAGGGACUGCAGAAAAUCCUGAACGU |
| | GCAGAAGAAGCUGCCCAUCAUCCAGAAAAUCAUCAUCAUGGACAG |
| | CAAGACCGACUACCAGGGCUUCCAGAGCAUGUACACCUUCGUGAC |
| | CAGCCAUCUGCCACCUGGCUUCAACGAGUACGACUUCGUGCCCGA |
| | GAGCUUCGACCGGGACAAGACAAUCGCCCUGAUCAUGAACAGCAG |
| | CGGCUCUACCGGACUGCCCAAAGGUGUUGCCCUGCCUCAUAGAAC |
| | UGCCUGCGUGCGGUUUAGCCACGCCAGAGAUCCCAUCUUCGGCAA |
| | CCAGAUCAUCCCCGACACAGCCAUCCUGAGCGUGGUGCCUUUUCA |
| | CCACGGCUUCGGCAUGUUCACCACACUGGGCUACCUGAUCUGCGG |
| | CUUCAGAGUGGUGCUGAUGUACCGCUUCGAGGAAGAACUGUUCCU |
| | GCGGAGCCUGCAGGACUACAAGAUCCAGUCUGCUCUGCUGGUGCC |
| | UACUCUGUUCAGCUUCUUUGCCAAGAGCACCCUGAUCGAUAAGUA |
| | CGACCUGAGCAACCUGCACGAGAUCGCCUCUGGCGGAGCCCCUCU |
| | GUCUAAAGAAGUGGGAGAAGCCGUCGCCAAGCGGUUCCAUCUGCC |
| | UGGCAUCAGACAAGGCUAUGGCCUGACCGAGACAACCAGCGCCAU |
| | UCUGAUUACCCCUGAGGGCGACGAUAAGCCUGGCGCUGUGGGAAA |
| | AGUGGUGCCAUUCUUCGAGGCCAAGGUGGUGGAUCUGGACACCGG |
| | CAAAACACUGGGCGUUAACCAGAGGGGCGAGCUGUGUGUUAGAGG |
| | CCCUAUGAUCAUGAGCGGCUACGUGAACAACCCCGAGGCCACAAA |
| | CGCUCUGAUCGACAAGGAUGGAUGGCUGCACAGCGGCGACAUUGC |
| | CUACUGGGACGAAGAUGAGCACUUCUUCAUCGUGGACCGGCUGAA |
| | GUCCCUGAUCAAGUACAAGGGCUACCAGGUGGCCCCAGCCGAGCU |
| | GGAAUCUAUUCUGCUGCAACACCCCAACAUCUUCGAUGCCGGCGU |
| | UGCAGGACUGCCCGAUGAUGAUGCUGGCGAACUGCCAGCUGCUGU |
| | GGUGGUGCUGGAACACGGCAAGACCAUGACCGAGAAAGAAAUCGU |
| | GGACUACGUGGCCAGCCAAGUGACCACCGCCAAGAAACUGAGAGG |
| | CGGCGUGGUGUUUGUGGACGAGGUGCCAAAAGGCCUGACAGGCAA |
| | GCUGGACGCCCGGAAGAUCAGAGAGAUCCUGAUUAAGGCCAAGAA |
| | AGGCGGCAAGAUCGCCGUGUAAUAGCUAACUAGACAAAACACCAA |
| | ACAAACAAGCUGCGAGCCACGGACUU (SEQ ID NO.: 23) |
| ORNA3A | AAAAUCCGUGGCUCGCAGCAAACACCAAACUAACGUCGAAAACAA |
| | ACACAAACCAACAAAAAGUGGCCACGCCCGGGCCACCGAUACUUC |
| | CCUUCACUCCUUCGGGACUGUUGGGGAGGAACACAACAGGGCUCC |
| | CCUGUUUUCCCAUUCCUUCCCCCUUUUCCCAACCCCAACCGCCGUA |
| | UCUGGUGGCGGCAAGACACACGGGUCUUUCCCUCUAAAGCACAAU |
| | UGUGUGUGUGUCCCAGGUCCUCCUGCGUACGGUGCGGGAGUGCUC |
| | CCACCCAACUGUUGUAAGCCUGUCCAACGCGUCGUCCUGGCAAGA |
| | CUAUGACGUCGCAUGUUCCGCUGCGGAUGCCGACCGGGUAACCGG |
| | UUCCCCAGUGUGUGUAGUGCGAUCUUCCAGGUCCUCCUGGUUGGC |
| | GUUGUCCAGAAACUGCUUCAGGUAAGUGGGGUGUGCCCAAUCCCU |
| | ACAAAGGUUGAUUCUUUCACCACCUUAGGAAUGCUCCGGAGGUAC |
| | CCCAGCAACAGCUGGGAUCUGACCGGAGGCUAAUUGUCUACGGGU |
| | GGUGUUUCCUUUUUCUUUUCACACAACUGUCACGAUGGAAGAUGC |
| | CAAGAACAUCAAGAAGGGCCCUGCUCCAUUCUACCCUCUGGAAGA |
| | UGGAACAGCCGGCGAGCAGCUGCACAAGGCCAUGAAGAGAUAUGC |
| | CCUGGUGCCUGGCACAAUCGCCUUCACAGAUGCCCACAUCGAGGU |
| | GGACAUCACCUACGCCGAGUACUUCGAGAUGUCUGUGCGGCUGGC |
| | CGAAGCUAUGAAGCGCUACGGCCUGAACACCAACCACCGGAUCGU |
| | CGUGUGCAGCGAGAACAGCCUGCAGUUCUUCAUGCCUGUGCUGGG |
| | CGCCCUGUUCAUCGGAGUUGCUGUGGCCCCUGCCAACGACAUCUA |
| | CAACGAGCGCGAGCUGCUGAACAGCAUGGGCAUCUCUCAGCCCAC |
| | CGUGGUGUUCGUGUCCAAGAAGGGACUGCAGAAAAUCCUGAACGU |
| | GCAGAAGAAGCUGCCCAUCAUCCAGAAAAUCAUCAUCAUGGACAG |
| | CAAGACCGACUACCAGGGCUUCCAGAGCAUGUACACCUUCGUGAC |
| | CAGCCAUCUGCCACCUGGCUUCAACGAGUACGACUUCGUGCCCGA |
| | GAGCUUCGACCGGGACAAGACAAUCGCCCUGAUCAUGAACAGCAG |
| | CGGCUCUACCGGACUGCCCAAAGGUGUUGCCCUGCCUCAUAGAAC |
| | UGCCUGCGUGCGGUUUAGCCACGCCAGAGAUCCCAUCUUCGGCAA |
| | CCAGAUCAUCCCCGACACAGCCAUCCUGAGCGUGGUGCCUUUUCA |
| | CCACGGCUUCGGCAUGUUCACCACACUGGGCUACCUGAUCUGCGG |
| | CUUCAGAGUGGUGCUGAUGUACCGCUUCGAGGAAGAACUGUUCCU |
| | GCGGAGCCUGCAGGACUACAAGAUCCAGUCUGCUCUGCUGGUGCC |
| | UACUCUGUUCAGCUUCUUUGCCAAGAGCACCCUGAUCGAUAAGUA |
| | CGACCUGAGCAACCUGCACGAGAUCGCCUCUGGCGGAGCCCCUCU |
| | GUCUAAAGAAGUGGGAGAAGCCGUCGCCAAGCGGUUCCAUCUGCC |
| | UGGCAUCAGACAAGGCUAUGGCCUGACCGAGACAACCAGCGCCAU |
| | UCUGAUUACCCCUGAGGGCGACGAUAAGCCUGGCGCUGUGGGAAA |
| | AGUGGUGCCAUUCUUCGAGGCCAAGGUGGUGGAUCUGGACACCGG |
| | CAAAACACUGGGCGUUAACCAGAGGGGCGAGCUGUGUGUUAGAGG |
| | CCCUAUGAUCAUGAGCGGCUACGUGAACAACCCCGAGGCCACAAA |
| | CGCUCUGAUCGACAAGGAUGGAUGGCUGCACAGCGGCGACAUUGC |
| | CUACUGGGACGAAGAUGAGCACUUCUUCAUCGUGGACCGGCUGAA |
| | GUCCCUGAUCAAGUACAAGGGCUACCAGGUGGCCCCAGCCGAGCU |
| | GGAAUCUAUUCUGCUGCAACACCCCAACAUCUUCGAUGCCGGCGU |
| | UGCAGGACUGCCCGAUGAUGAUGCUGGCGAACUGCCAGCUGCUGU |
| | GGUGGUGCUGGAACACGGCAAGACCAUGACCGAGAAAGAAAUCGU |
| | GGACUACGUGGCCAGCCAAGUGACCACCGCCAAGAAACUGAGAGG |
| | CGGCGUGGUGUUUGUGGACGAGGUGCCAAAAGGCCUGACAGGCAA |
| | GCUGGACGCCCGGAAGAUCAGAGAGAUCCUGAUUAAGGCCAAGAA |
| | AGGCGGCAAGAUCGCCGUGUAAUAGCUAACUAGACAAAACACCAA |
| | ACAAACAAGCUGCGAGCCACGGACUU (SEQ ID NO.: 24) |
| ORNA4A | AAAAUCCGUGGCUCGCAGCAAACACCAAACAAGCAUCGAAAACAA |
| | ACACAAACCAACAAAAAGUGGCCACGCCCGGGCCACCGAUACUUC |
| | CCUUCACUCCUUCGGGACUGUUGGGGAGGAACACAACAGGGCUCC |
| | CCUGUUUUCCCAUUCCUUCCCCCUUUUCCCAACCCCAACCGCCGUA |
| | UCUGGUGGCGGCAAGACACACGGGUCUUUCCCUCUAAAGCACAAU |
| | UGUGUGUGUGUCCCAGGUCCUCCUGCGUACGGUGCGGGAGUGCUC |
| | CCACCCAACUGUUGUAAGCCUGUCCAACGCGUCGUCCUGGCAAGA |
| | CUAUGACGUCGCAUGUUCCGCUGCGGAUGCCGACCGGGUAACCGG |
| | UUCCCCAGUGUGUGUAGUGCGAUCUUCCAGGUCCUCCUGGUUGGC |
| | GUUGUCCAGAAACUGCUUCAGGUAAGUGGGGUGUGCCCAAUCCCU |
| | ACAAAGGUUGAUUCUUUCACCACCUUAGGAAUGCUCCGGAGGUAC |
| | CCCAGCAACAGCUGGGAUCUGACCGGAGGCUAAUUGUCUACGGGU |
| | GGUGUUUCCUUUUUCUUUUCACACAACUGUCACGAUGGAAGAUGC |
| | CAAGAACAUCAAGAAGGGCCCUGCUCCAUUCUACCCUCUGGAAGA |
| | UGGAACAGCCGGCGAGCAGCUGCACAAGGCCAUGAAGAGAUAUGC |
| | CCUGGUGCCUGGCACAAUCGCCUUCACAGAUGCCCACAUCGAGGU |
| | GGACAUCACCUACGCCGAGUACUUCGAGAUGUCUGUGCGGCUGGC |
| | CGAAGCUAUGAAGCGCUACGGCCUGAACACCAACCACCGGAUCGU |
| | CGUGUGCAGCGAGAACAGCCUGCAGUUCUUCAUGCCUGUGCUGGG |
| | CGCCCUGUUCAUCGGAGUUGCUGUGGCCCCUGCCAACGACAUCUA |
| | CAACGAGCGCGAGCUGCUGAACAGCAUGGGCAUCUCUCAGCCCAC |
| | CGUGGUGUUCGUGUCCAAGAAGGGACUGCAGAAAAUCCUGAACGU |
| | GCAGAAGAAGCUGCCCAUCAUCCAGAAAAUCAUCAUCAUGGACAG |
| | CAAGACCGACUACCAGGGCUUCCAGAGCAUGUACACCUUCGUGAC |
| | CAGCCAUCUGCCACCUGGCUUCAACGAGUACGACUUCGUGCCCGA |
| | GAGCUUCGACCGGGACAAGACAAUCGCCCUGAUCAUGAACAGCAG |
| | CGGCUCUACCGGACUGCCCAAAGGUGUUGCCCUGCCUCAUAGAAC |
| | UGCCUGCGUGCGGUUUAGCCACGCCAGAGAUCCCAUCUUCGGCAA |
| | CCAGAUCAUCCCCGACACAGCCAUCCUGAGCGUGGUGCCUUUUCA |
| | CCACGGCUUCGGCAUGUUCACCACACUGGGCUACCUGAUCUGCGG |
| | CUUCAGAGUGGUGCUGAUGUACCGCUUCGAGGAAGAACUGUUCCU |
| | GCGGAGCCUGCAGGACUACAAGAUCCAGUCUGCUCUGCUGGUGCC |
| | UACUCUGUUCAGCUUCUUUGCCAAGAGCACCCUGAUCGAUAAGUA |
| | CGACCUGAGCAACCUGCACGAGAUCGCCUCUGGCGGAGCCCCUCU |
| | GUCUAAAGAAGUGGGAGAAGCCGUCGCCAAGCGGUUCCAUCUGCC |
| | UGGCAUCAGACAAGGCUAUGGCCUGACCGAGACAACCAGCGCCAU |
| | UCUGAUUACCCCUGAGGGCGACGAUAAGCCUGGCGCUGUGGGAAA |
| | AGUGGUGCCAUUCUUCGAGGCCAAGGUGGUGGAUCUGGACACCGG |
| | CAAAACACUGGGCGUUAACCAGAGGGGCGAGCUGUGUGUUAGAGG |
| | CCCUAUGAUCAUGAGCGGCUACGUGAACAACCCCGAGGCCACAAA |
| | CGCUCUGAUCGACAAGGAUGGAUGGCUGCACAGCGGCGACAUUGC |
| | CUACUGGGACGAAGAUGAGCACUUCUUCAUCGUGGACCGGCUGAA |
| | GUCCCUGAUCAAGUACAAGGGCUACCAGGUGGCCCCAGCCGAGCU |
| | GGAAUCUAUUCUGCUGCAACACCCCAACAUCUUCGAUGCCGGCGU |
| | UGCAGGACUGCCCGAUGAUGAUGCUGGCGAACUGCCAGCUGCUGU |
| | GGUGGUGCUGGAACACGGCAAGACCAUGACCGAGAAAGAAAUCGU |
| | GGACUACGUGGCCAGCCAAGUGACCACCGCCAAGAAACUGAGAGG |
| | CGGCGUGGUGUUUGUGGACGAGGUGCCAAAAGGCCUGACAGGCAA |
| | GCUGGACGCCCGGAAGAUCAGAGAGAUCCUGAUUAAGGCCAAGAA |
| | AGGCGGCAAGAUCGCCGUGUAAUAGCUAACUAGACAAAACACCAA |
| | ACAAACAAGCUGCGAGCCACGGACUU (SEQ ID NO.: 25) |
| ORNA5A | AAAAUCCGUGGCUCGCAGCAAACACCAAACGCAAUUCCAAAACAA |
| | ACACAAACCAACAAAAAGUGGCCACGCCCGGGCCACCGAUACUUC |
| | CCUUCACUCCUUCGGGACUGUUGGGGAGGAACACAACAGGGCUCC |
| | CCUGUUUUCCCAUUCCUUCCCCCUUUUCCCAACCCCAACCGCCGUA |
| | UCUGGUGGCGGCAAGACACACGGGUCUUUCCCUCUAAAGCACAAU |
| | UGUGUGUGUGUCCCAGGUCCUCCUGCGUACGGUGCGGGAGUGCUC |
| | CCACCCAACUGUUGUAAGCCUGUCCAACGCGUCGUCCUGGCAAGA |
| | CUAUGACGUCGCAUGUUCCGCUGCGGAUGCCGACCGGGUAACCGG |
| | UUCCCCAGUGUGUGUAGUGCGAUCUUCCAGGUCCUCCUGGUUGGC |
| | GUUGUCCAGAAACUGCUUCAGGUAAGUGGGGUGUGCCCAAUCCCU |
| | ACAAAGGUUGAUUCUUUCACCACCUUAGGAAUGCUCCGGAGGUAC |
| | CCCAGCAACAGCUGGGAUCUGACCGGAGGCUAAUUGUCUACGGGU |
| | GGUGUUUCCUUUUUCUUUUCACACAACUGUCACGAUGGAAGAUGC |
| | CAAGAACAUCAAGAAGGGCCCUGCUCCAUUCUACCCUCUGGAAGA |
| | UGGAACAGCCGGCGAGCAGCUGCACAAGGCCAUGAAGAGAUAUGC |
| | CCUGGUGCCUGGCACAAUCGCCUUCACAGAUGCCCACAUCGAGGU |
| | GGACAUCACCUACGCCGAGUACUUCGAGAUGUCUGUGCGGCUGGC |
| | CGAAGCUAUGAAGCGCUACGGCCUGAACACCAACCACCGGAUCGU |
| | CGUGUGCAGCGAGAACAGCCUGCAGUUCUUCAUGCCUGUGCUGGG |
| | CGCCCUGUUCAUCGGAGUUGCUGUGGCCCCUGCCAACGACAUCUA |
| | CAACGAGCGCGAGCUGCUGAACAGCAUGGGCAUCUCUCAGCCCAC |
| | CGUGGUGUUCGUGUCCAAGAAGGGACUGCAGAAAAUCCUGAACGU |
| | GCAGAAGAAGCUGCCCAUCAUCCAGAAAAUCAUCAUCAUGGACAG |
| | CAAGACCGACUACCAGGGCUUCCAGAGCAUGUACACCUUCGUGAC |
| | CAGCCAUCUGCCACCUGGCUUCAACGAGUACGACUUCGUGCCCGA |
| | GAGCUUCGACCGGGACAAGACAAUCGCCCUGAUCAUGAACAGCAG |
| | CGGCUCUACCGGACUGCCCAAAGGUGUUGCCCUGCCUCAUAGAAC |
| | UGCCUGCGUGCGGUUUAGCCACGCCAGAGAUCCCAUCUUCGGCAA |
| | CCAGAUCAUCCCCGACACAGCCAUCCUGAGCGUGGUGCCUUUUCA |
| | CCACGGCUUCGGCAUGUUCACCACACUGGGCUACCUGAUCUGCGG |
| | CUUCAGAGUGGUGCUGAUGUACCGCUUCGAGGAAGAACUGUUCCU |
| | GCGGAGCCUGCAGGACUACAAGAUCCAGUCUGCUCUGCUGGUGCC |
| | UACUCUGUUCAGCUUCUUUGCCAAGAGCACCCUGAUCGAUAAGUA |
| | CGACCUGAGCAACCUGCACGAGAUCGCCUCUGGCGGAGCCCCUCU |
| | GUCUAAAGAAGUGGGAGAAGCCGUCGCCAAGCGGUUCCAUCUGCC |
| | UGGCAUCAGACAAGGCUAUGGCCUGACCGAGACAACCAGCGCCAU |
| | UCUGAUUACCCCUGAGGGCGACGAUAAGCCUGGCGCUGUGGGAAA |
| | AGUGGUGCCAUUCUUCGAGGCCAAGGUGGUGGAUCUGGACACCGG |
| | CAAAACACUGGGCGUUAACCAGAGGGGCGAGCUGUGUGUUAGAGG |
| | CCCUAUGAUCAUGAGCGGCUACGUGAACAACCCCGAGGCCACAAA |
| | CGCUCUGAUCGACAAGGAUGGAUGGCUGCACAGCGGCGACAUUGC |
| | CUACUGGGACGAAGAUGAGCACUUCUUCAUCGUGGACCGGCUGAA |
| | GUCCCUGAUCAAGUACAAGGGCUACCAGGUGGCCCCAGCCGAGCU |
| | GGAAUCUAUUCUGCUGCAACACCCCAACAUCUUCGAUGCCGGCGU |
| | UGCAGGACUGCCCGAUGAUGAUGCUGGCGAACUGCCAGCUGCUGU |
| | GGUGGUGCUGGAACACGGCAAGACCAUGACCGAGAAAGAAAUCGU |
| | GGACUACGUGGCCAGCCAAGUGACCACCGCCAAGAAACUGAGAGG |
| | CGGCGUGGUGUUUGUGGACGAGGUGCCAAAAGGCCUGACAGGCAA |
| | GCUGGACGCCCGGAAGAUCAGAGAGAUCCUGAUUAAGGCCAAGAA |
| | AGGCGGCAAGAUCGCCGUGUAAUAGCUAACUAGACAAAACACCAA |
| | ACAAACAAGCUGCGAGCCACGGACUU (SEQ ID NO.: 26) |
| ORNA6A | AAAAUCCGUGGCUCGCAGCAAACACCAAACGCUACAACAAAACAA |
| | ACACAAACCAACAAAAAGUGGCCACGCCCGGGCCACCGAUACUUC |
| | CCUUCACUCCUUCGGGACUGUUGGGGAGGAACACAACAGGGCUCC |
| | CCUGUUUUCCCAUUCCUUCCCCCUUUUCCCAACCCCAACCGCCGUA |
| | UCUGGUGGCGGCAAGACACACGGGUCUUUCCCUCUAAAGCACAAU |
| | UGUGUGUGUGUCCCAGGUCCUCCUGCGUACGGUGCGGGAGUGCUC |
| | CCACCCAACUGUUGUAAGCCUGUCCAACGCGUCGUCCUGGCAAGA |
| | CUAUGACGUCGCAUGUUCCGCUGCGGAUGCCGACCGGGUAACCGG |
| | UUCCCCAGUGUGUGUAGUGCGAUCUUCCAGGUCCUCCUGGUUGGC |
| | GUUGUCCAGAAACUGCUUCAGGUAAGUGGGGUGUGCCCAAUCCCU |
| | ACAAAGGUUGAUUCUUUCACCACCUUAGGAAUGCUCCGGAGGUAC |
| | CCCAGCAACAGCUGGGAUCUGACCGGAGGCUAAUUGUCUACGGGU |
| | GGUGUUUCCUUUUUCUUUUCACACAACUGUCACGAUGGAAGAUGC |
| | CAAGAACAUCAAGAAGGGCCCUGCUCCAUUCUACCCUCUGGAAGA |
| | UGGAACAGCCGGCGAGCAGCUGCACAAGGCCAUGAAGAGAUAUGC |
| | CCUGGUGCCUGGCACAAUCGCCUUCACAGAUGCCCACAUCGAGGU |
| | GGACAUCACCUACGCCGAGUACUUCGAGAUGUCUGUGCGGCUGGC |
| | CGAAGCUAUGAAGCGCUACGGCCUGAACACCAACCACCGGAUCGU |
| | CGUGUGCAGCGAGAACAGCCUGCAGUUCUUCAUGCCUGUGCUGGG |
| | CGCCCUGUUCAUCGGAGUUGCUGUGGCCCCUGCCAACGACAUCUA |
| | CAACGAGCGCGAGCUGCUGAACAGCAUGGGCAUCUCUCAGCCCAC |
| | CGUGGUGUUCGUGUCCAAGAAGGGACUGCAGAAAAUCCUGAACGU |
| | GCAGAAGAAGCUGCCCAUCAUCCAGAAAAUCAUCAUCAUGGACAG |
| | CAAGACCGACUACCAGGGCUUCCAGAGCAUGUACACCUUCGUGAC |
| | CAGCCAUCUGCCACCUGGCUUCAACGAGUACGACUUCGUGCCCGA |
| | GAGCUUCGACCGGGACAAGACAAUCGCCCUGAUCAUGAACAGCAG |
| | CGGCUCUACCGGACUGCCCAAAGGUGUUGCCCUGCCUCAUAGAAC |
| | UGCCUGCGUGCGGUUUAGCCACGCCAGAGAUCCCAUCUUCGGCAA |
| | CCAGAUCAUCCCCGACACAGCCAUCCUGAGCGUGGUGCCUUUUCA |
| | CCACGGCUUCGGCAUGUUCACCACACUGGGCUACCUGAUCUGCGG |
| | CUUCAGAGUGGUGCUGAUGUACCGCUUCGAGGAAGAACUGUUCCU |
| | GCGGAGCCUGCAGGACUACAAGAUCCAGUCUGCUCUGCUGGUGCC |
| | UACUCUGUUCAGCUUCUUUGCCAAGAGCACCCUGAUCGAUAAGUA |
| | CGACCUGAGCAACCUGCACGAGAUCGCCUCUGGCGGAGCCCCUCU |
| | GUCUAAAGAAGUGGGAGAAGCCGUCGCCAAGCGGUUCCAUCUGCC |
| | UGGCAUCAGACAAGGCUAUGGCCUGACCGAGACAACCAGCGCCAU |
| | UCUGAUUACCCCUGAGGGCGACGAUAAGCCUGGCGCUGUGGGAAA |
| | AGUGGUGCCAUUCUUCGAGGCCAAGGUGGUGGAUCUGGACACCGG |
| | CAAAACACUGGGCGUUAACCAGAGGGGCGAGCUGUGUGUUAGAGG |
| | CCCUAUGAUCAUGAGCGGCUACGUGAACAACCCCGAGGCCACAAA |
| | CGCUCUGAUCGACAAGGAUGGAUGGCUGCACAGCGGCGACAUUGC |
| | CUACUGGGACGAAGAUGAGCACUUCUUCAUCGUGGACCGGCUGAA |
| | GUCCCUGAUCAAGUACAAGGGCUACCAGGUGGCCCCAGCCGAGCU |
| | GGAAUCUAUUCUGCUGCAACACCCCAACAUCUUCGAUGCCGGCGU |
| | UGCAGGACUGCCCGAUGAUGAUGCUGGCGAACUGCCAGCUGCUGU |
| | GGUGGUGCUGGAACACGGCAAGACCAUGACCGAGAAAGAAAUCGU |
| | GGACUACGUGGCCAGCCAAGUGACCACCGCCAAGAAACUGAGAGG |
| | CGGCGUGGUGUUUGUGGACGAGGUGCCAAAAGGCCUGACAGGCAA |
| | GCUGGACGCCCGGAAGAUCAGAGAGAUCCUGAUUAAGGCCAAGAA |
| | AGGCGGCAAGAUCGCCGUGUAAUAGCUAACUAGACAAAACACCAA |
| | ACAAACAAGCUGCGAGCCACGGACUU (SEQ ID NO.: 27) |
| ORNA7A | AAAAUCCGUGGCUCGCAGCAAACACCAAACUAGGAGCUAAAACAA |
| | ACACAAACCAACAAAAAGUGGCCACGCCCGGGCCACCGAUACUUC |
| | CCUUCACUCCUUCGGGACUGUUGGGGAGGAACACAACAGGGCUCC |
| | CCUGUUUUCCCAUUCCUUCCCCCUUUUCCCAACCCCAACCGCCGUA |
| | UCUGGUGGCGGCAAGACACACGGGUCUUUCCCUCUAAAGCACAAU |
| | UGUGUGUGUGUCCCAGGUCCUCCUGCGUACGGUGCGGGAGUGCUC |
| | CCACCCAACUGUUGUAAGCCUGUCCAACGCGUCGUCCUGGCAAGA |
| | CUAUGACGUCGCAUGUUCCGCUGCGGAUGCCGACCGGGUAACCGG |
| | UUCCCCAGUGUGUGUAGUGCGAUCUUCCAGGUCCUCCUGGUUGGC |
| | GUUGUCCAGAAACUGCUUCAGGUAAGUGGGGUGUGCCCAAUCCCU |
| | ACAAAGGUUGAUUCUUUCACCACCUUAGGAAUGCUCCGGAGGUAC |
| | CCCAGCAACAGCUGGGAUCUGACCGGAGGCUAAUUGUCUACGGGU |
| | GGUGUUUCCUUUUUCUUUUCACACAACUGUCACGAUGGAAGAUGC |
| | CAAGAACAUCAAGAAGGGCCCUGCUCCAUUCUACCCUCUGGAAGA |
| | UGGAACAGCCGGCGAGCAGCUGCACAAGGCCAUGAAGAGAUAUGC |
| | CCUGGUGCCUGGCACAAUCGCCUUCACAGAUGCCCACAUCGAGGU |
| | GGACAUCACCUACGCCGAGUACUUCGAGAUGUCUGUGCGGCUGGC |
| | CGAAGCUAUGAAGCGCUACGGCCUGAACACCAACCACCGGAUCGU |
| | CGUGUGCAGCGAGAACAGCCUGCAGUUCUUCAUGCCUGUGCUGGG |
| | CGCCCUGUUCAUCGGAGUUGCUGUGGCCCCUGCCAACGACAUCUA |
| | CAACGAGCGCGAGCUGCUGAACAGCAUGGGCAUCUCUCAGCCCAC |
| | CGUGGUGUUCGUGUCCAAGAAGGGACUGCAGAAAAUCCUGAACGU |
| | GCAGAAGAAGCUGCCCAUCAUCCAGAAAAUCAUCAUCAUGGACAG |
| | CAAGACCGACUACCAGGGCUUCCAGAGCAUGUACACCUUCGUGAC |
| | CAGCCAUCUGCCACCUGGCUUCAACGAGUACGACUUCGUGCCCGA |
| | GAGCUUCGACCGGGACAAGACAAUCGCCCUGAUCAUGAACAGCAG |
| | CGGCUCUACCGGACUGCCCAAAGGUGUUGCCCUGCCUCAUAGAAC |
| | UGCCUGCGUGCGGUUUAGCCACGCCAGAGAUCCCAUCUUCGGCAA |
| | CCAGAUCAUCCCCGACACAGCCAUCCUGAGCGUGGUGCCUUUUCA |
| | CCACGGCUUCGGCAUGUUCACCACACUGGGCUACCUGAUCUGCGG |
| | CUUCAGAGUGGUGCUGAUGUACCGCUUCGAGGAAGAACUGUUCCU |
| | GCGGAGCCUGCAGGACUACAAGAUCCAGUCUGCUCUGCUGGUGCC |
| | UACUCUGUUCAGCUUCUUUGCCAAGAGCACCCUGAUCGAUAAGUA |
| | CGACCUGAGCAACCUGCACGAGAUCGCCUCUGGCGGAGCCCCUCU |
| | GUCUAAAGAAGUGGGAGAAGCCGUCGCCAAGCGGUUCCAUCUGCC |
| | UGGCAUCAGACAAGGCUAUGGCCUGACCGAGACAACCAGCGCCAU |
| | UCUGAUUACCCCUGAGGGCGACGAUAAGCCUGGCGCUGUGGGAAA |
| | AGUGGUGCCAUUCUUCGAGGCCAAGGUGGUGGAUCUGGACACCGG |
| | CAAAACACUGGGCGUUAACCAGAGGGGCGAGCUGUGUGUUAGAGG |
| | CCCUAUGAUCAUGAGCGGCUACGUGAACAACCCCGAGGCCACAAA |
| | CGCUCUGAUCGACAAGGAUGGAUGGCUGCACAGCGGCGACAUUGC |
| | CUACUGGGACGAAGAUGAGCACUUCUUCAUCGUGGACCGGCUGAA |
| | GUCCCUGAUCAAGUACAAGGGCUACCAGGUGGCCCCAGCCGAGCU |
| | GGAAUCUAUUCUGCUGCAACACCCCAACAUCUUCGAUGCCGGCGU |
| | UGCAGGACUGCCCGAUGAUGAUGCUGGCGAACUGCCAGCUGCUGU |
| | GGUGGUGCUGGAACACGGCAAGACCAUGACCGAGAAAGAAAUCGU |
| | GGACUACGUGGCCAGCCAAGUGACCACCGCCAAGAAACUGAGAGG |
| | CGGCGUGGUGUUUGUGGACGAGGUGCCAAAAGGCCUGACAGGCAA |
| | GCUGGACGCCCGGAAGAUCAGAGAGAUCCUGAUUAAGGCCAAGAA |
| | AGGCGGCAAGAUCGCCGUGUAAUAGCUAACUAGACAAAACACCAA |
| | ACAAACAAGCUGCGAGCCACGGACUU (SEQ ID NO.: 28) |
| ORNA8A | AAAAUCCGUGGCUCGCAGCAAACACCAAACUGGAUGGUAAAACAA |
| | ACACAAACCAACAAAAAGUGGCCACGCCCGGGCCACCGAUACUUC |
| | CCUUCACUCCUUCGGGACUGUUGGGGAGGAACACAACAGGGCUCC |
| | CCUGUUUUCCCAUUCCUUCCCCCUUUUCCCAACCCCAACCGCCGUA |
| | UCUGGUGGCGGCAAGACACACGGGUCUUUCCCUCUAAAGCACAAU |
| | UGUGUGUGUGUCCCAGGUCCUCCUGCGUACGGUGCGGGAGUGCUC |
| | CCACCCAACUGUUGUAAGCCUGUCCAACGCGUCGUCCUGGCAAGA |
| | CUAUGACGUCGCAUGUUCCGCUGCGGAUGCCGACCGGGUAACCGG |
| | UUCCCCAGUGUGUGUAGUGCGAUCUUCCAGGUCCUCCUGGUUGGC |
| | GUUGUCCAGAAACUGCUUCAGGUAAGUGGGGUGUGCCCAAUCCCU |
| | ACAAAGGUUGAUUCUUUCACCACCUUAGGAAUGCUCCGGAGGUAC |
| | CCCAGCAACAGCUGGGAUCUGACCGGAGGCUAAUUGUCUACGGGU |
| | GGUGUUUCCUUUUUCUUUUCACACAACUGUCACGAUGGAAGAUGC |
| | CAAGAACAUCAAGAAGGGCCCUGCUCCAUUCUACCCUCUGGAAGA |
| | UGGAACAGCCGGCGAGCAGCUGCACAAGGCCAUGAAGAGAUAUGC |
| | CCUGGUGCCUGGCACAAUCGCCUUCACAGAUGCCCACAUCGAGGU |
| | GGACAUCACCUACGCCGAGUACUUCGAGAUGUCUGUGCGGCUGGC |
| | CGAAGCUAUGAAGCGCUACGGCCUGAACACCAACCACCGGAUCGU |
| | CGUGUGCAGCGAGAACAGCCUGCAGUUCUUCAUGCCUGUGCUGGG |
| | CGCCCUGUUCAUCGGAGUUGCUGUGGCCCCUGCCAACGACAUCUA |
| | CAACGAGCGCGAGCUGCUGAACAGCAUGGGCAUCUCUCAGCCCAC |
| | CGUGGUGUUCGUGUCCAAGAAGGGACUGCAGAAAAUCCUGAACGU |
| | GCAGAAGAAGCUGCCCAUCAUCCAGAAAAUCAUCAUCAUGGACAG |
| | CAAGACCGACUACCAGGGCUUCCAGAGCAUGUACACCUUCGUGAC |
| | CAGCCAUCUGCCACCUGGCUUCAACGAGUACGACUUCGUGCCCGA |
| | GAGCUUCGACCGGGACAAGACAAUCGCCCUGAUCAUGAACAGCAG |
| | CGGCUCUACCGGACUGCCCAAAGGUGUUGCCCUGCCUCAUAGAAC |
| | UGCCUGCGUGCGGUUUAGCCACGCCAGAGAUCCCAUCUUCGGCAA |
| | CCAGAUCAUCCCCGACACAGCCAUCCUGAGCGUGGUGCCUUUUCA |
| | CCACGGCUUCGGCAUGUUCACCACACUGGGCUACCUGAUCUGCGG |
| | CUUCAGAGUGGUGCUGAUGUACCGCUUCGAGGAAGAACUGUUCCU |
| | GCGGAGCCUGCAGGACUACAAGAUCCAGUCUGCUCUGCUGGUGCC |
| | UACUCUGUUCAGCUUCUUUGCCAAGAGCACCCUGAUCGAUAAGUA |
| | CGACCUGAGCAACCUGCACGAGAUCGCCUCUGGCGGAGCCCCUCU |
| | GUCUAAAGAAGUGGGAGAAGCCGUCGCCAAGCGGUUCCAUCUGCC |
| | UGGCAUCAGACAAGGCUAUGGCCUGACCGAGACAACCAGCGCCAU |
| | UCUGAUUACCCCUGAGGGCGACGAUAAGCCUGGCGCUGUGGGAAA |
| | AGUGGUGCCAUUCUUCGAGGCCAAGGUGGUGGAUCUGGACACCGG |
| | CAAAACACUGGGCGUUAACCAGAGGGGCGAGCUGUGUGUUAGAGG |
| | CCCUAUGAUCAUGAGCGGCUACGUGAACAACCCCGAGGCCACAAA |
| | CGCUCUGAUCGACAAGGAUGGAUGGCUGCACAGCGGCGACAUUGC |
| | CUACUGGGACGAAGAUGAGCACUUCUUCAUCGUGGACCGGCUGAA |
| | GUCCCUGAUCAAGUACAAGGGCUACCAGGUGGCCCCAGCCGAGCU |
| | GGAAUCUAUUCUGCUGCAACACCCCAACAUCUUCGAUGCCGGCGU |
| | UGCAGGACUGCCCGAUGAUGAUGCUGGCGAACUGCCAGCUGCUGU |
| | GGUGGUGCUGGAACACGGCAAGACCAUGACCGAGAAAGAAAUCGU |
| | GGACUACGUGGCCAGCCAAGUGACCACCGCCAAGAAACUGAGAGG |
| | CGGCGUGGUGUUUGUGGACGAGGUGCCAAAAGGCCUGACAGGCAA |
| | GCUGGACGCCCGGAAGAUCAGAGAGAUCCUGAUUAAGGCCAAGAA |
| | AGGCGGCAAGAUCGCCGUGUAAUAGCUAACUAGACAAAACACCAA |
| | ACAAACAAGCUGCGAGCCACGGACUU (SEQ ID NO.: 29) |
| ORNA9A | AAAAUCCGUGGCUCGCAGCAAACACCAAACUACUAGCGAAAACAA |
| | ACACAAACCAACAAAAAGUGGCCACGCCCGGGCCACCGAUACUUC |
| | CCUUCACUCCUUCGGGACUGUUGGGGAGGAACACAACAGGGCUCC |
| | CCUGUUUUCCCAUUCCUUCCCCCUUUUCCCAACCCCAACCGCCGUA |
| | UCUGGUGGCGGCAAGACACACGGGUCUUUCCCUCUAAAGCACAAU |
| | UGUGUGUGUGUCCCAGGUCCUCCUGCGUACGGUGCGGGAGUGCUC |
| | CCACCCAACUGUUGUAAGCCUGUCCAACGCGUCGUCCUGGCAAGA |
| | CUAUGACGUCGCAUGUUCCGCUGCGGAUGCCGACCGGGUAACCGG |
| | UUCCCCAGUGUGUGUAGUGCGAUCUUCCAGGUCCUCCUGGUUGGC |
| | GUUGUCCAGAAACUGCUUCAGGUAAGUGGGGUGUGCCCAAUCCCU |
| | ACAAAGGUUGAUUCUUUCACCACCUUAGGAAUGCUCCGGAGGUAC |
| | CCCAGCAACAGCUGGGAUCUGACCGGAGGCUAAUUGUCUACGGGU |
| | GGUGUUUCCUUUUUUUUUCACACAACUGUCACGAUGGAAGAUGC |
| | CAAGAACAUCAAGAAGGGCCCUGCUCCAUUCUACCCUCUGGAAGA |
| | UGGAACAGCCGGCGAGCAGCUGCACAAGGCCAUGAAGAGAUAUGC |
| | CCUGGUGCCUGGCACAAUCGCCUUCACAGAUGCCCACAUCGAGGU |
| | GGACAUCACCUACGCCGAGUACUUCGAGAUGUCUGUGCGGCUGGC |
| | CGAAGCUAUGAAGCGCUACGGCCUGAACACCAACCACCGGAUCGU |
| | CGUGUGCAGCGAGAACAGCCUGCAGUUCUUCAUGCCUGUGCUGGG |
| | CGCCCUGUUCAUCGGAGUUGCUGUGGCCCCUGCCAACGACAUCUA |
| | CAACGAGCGCGAGCUGCUGAACAGCAUGGGCAUCUCUCAGCCCAC |
| | CGUGGUGUUCGUGUCCAAGAAGGGACUGCAGAAAAUCCUGAACGU |
| | GCAGAAGAAGCUGCCCAUCAUCCAGAAAAUCAUCAUCAUGGACAG |
| | CAAGACCGACUACCAGGGCUUCCAGAGCAUGUACACCUUCGUGAC |
| | CAGCCAUCUGCCACCUGGCUUCAACGAGUACGACUUCGUGCCCGA |
| | GAGCUUCGACCGGGACAAGACAAUCGCCCUGAUCAUGAACAGCAG |
| | CGGCUCUACCGGACUGCCCAAAGGUGUUGCCCUGCCUCAUAGAAC |
| | UGCCUGCGUGCGGUUUAGCCACGCCAGAGAUCCCAUCUUCGGCAA |
| | CCAGAUCAUCCCCGACACAGCCAUCCUGAGCGUGGUGCCUUUUCA |
| | CCACGGCUUCGGCAUGUUCACCACACUGGGCUACCUGAUCUGCGG |
| | CUUCAGAGUGGUGCUGAUGUACCGCUUCGAGGAAGAACUGUUCCU |
| | GCGGAGCCUGCAGGACUACAAGAUCCAGUCUGCUCUGCUGGUGCC |
| | UACUCUGUUCAGCUUCUUUGCCAAGAGCACCCUGAUCGAUAAGUA |
| | CGACCUGAGCAACCUGCACGAGAUCGCCUCUGGCGGAGCCCCUCU |
| | GUCUAAAGAAGUGGGAGAAGCCGUCGCCAAGCGGUUCCAUCUGCC |
| | UGGCAUCAGACAAGGCUAUGGCCUGACCGAGACAACCAGCGCCAU |
| | UCUGAUUACCCCUGAGGGCGACGAUAAGCCUGGCGCUGUGGGAAA |
| | AGUGGUGCCAUUCUUCGAGGCCAAGGUGGUGGAUCUGGACACCGG |
| | CAAAACACUGGGCGUUAACCAGAGGGGCGAGCUGUGUGUUAGAGG |
| | CCCUAUGAUCAUGAGCGGCUACGUGAACAACCCCGAGGCCACAAA |
| | CGCUCUGAUCGACAAGGAUGGAUGGCUGCACAGCGGCGACAUUGC |
| | CUACUGGGACGAAGAUGAGCACUUCUUCAUCGUGGACCGGCUGAA |
| | GUCCCUGAUCAAGUACAAGGGCUACCAGGUGGCCCCAGCCGAGCU |
| | GGAAUCUAUUCUGCUGCAACACCCCAACAUCUUCGAUGCCGGCGU |
| | UGCAGGACUGCCCGAUGAUGAUGCUGGCGAACUGCCAGCUGCUGU |
| | GGUGGUGCUGGAACACGGCAAGACCAUGACCGAGAAAGAAAUCGU |
| | GGACUACGUGGCCAGCCAAGUGACCACCGCCAAGAAACUGAGAGG |
| | CGGCGUGGUGUUUGUGGACGAGGUGCCAAAAGGCCUGACAGGCAA |
| | GCUGGACGCCCGGAAGAUCAGAGAGAUCCUGAUUAAGGCCAAGAA |
| | AGGCGGCAAGAUCGCCGUGUAAUAGCUAACUAGACAAAACACCAA |
| | ACAAACAAGCUGCGAGCCACGGACUU (SEQ ID NO.: 30) |
| ORNA10A | AAAAUCCGUGGCUCGCAGCAAACACCAAACCAGUGCUUAAAACAA |
| | ACACAAACCAACAAAAAGUGGCCACGCCCGGGCCACCGAUACUUC |
| | CCUUCACUCCUUCGGGACUGUUGGGGAGGAACACAACAGGGCUCC |
| | CCUGUUUUCCCAUUCCUUCCCCCUUUUCCCAACCCCAACCGCCGUA |
| | UCUGGUGGCGGCAAGACACACGGGUCUUUCCCUCUAAAGCACAAU |
| | UGUGUGUGUGUCCCAGGUCCUCCUGCGUACGGUGCGGGAGUGCUC |
| | CCACCCAACUGUUGUAAGCCUGUCCAACGCGUCGUCCUGGCAAGA |
| | CUAUGACGUCGCAUGUUCCGCUGCGGAUGCCGACCGGGUAACCGG |
| | UUCCCCAGUGUGUGUAGUGCGAUCUUCCAGGUCCUCCUGGUUGGC |
| | GUUGUCCAGAAACUGCUUCAGGUAAGUGGGGUGUGCCCAAUCCCU |
| | ACAAAGGUUGAUUCUUUCACCACCUUAGGAAUGCUCCGGAGGUAC |
| | CCCAGCAACAGCUGGGAUCUGACCGGAGGCUAAUUGUCUACGGGU |
| | GGUGUUUCCUUUUUCUUUUCACACAACUGUCACGAUGGAAGAUGC |
| | CAAGAACAUCAAGAAGGGCCCUGCUCCAUUCUACCCUCUGGAAGA |
| | UGGAACAGCCGGCGAGCAGCUGCACAAGGCCAUGAAGAGAUAUGC |
| | CCUGGUGCCUGGCACAAUCGCCUUCACAGAUGCCCACAUCGAGGU |
| | GGACAUCACCUACGCCGAGUACUUCGAGAUGUCUGUGCGGCUGGC |
| | CGAAGCUAUGAAGCGCUACGGCCUGAACACCAACCACCGGAUCGU |
| | CGUGUGCAGCGAGAACAGCCUGCAGUUCUUCAUGCCUGUGCUGGG |
| | CGCCCUGUUCAUCGGAGUUGCUGUGGCCCCUGCCAACGACAUCUA |
| | CAACGAGCGCGAGCUGCUGAACAGCAUGGGCAUCUCUCAGCCCAC |
| | CGUGGUGUUCGUGUCCAAGAAGGGACUGCAGAAAAUCCUGAACGU |
| | GCAGAAGAAGCUGCCCAUCAUCCAGAAAAUCAUCAUCAUGGACAG |
| | CAAGACCGACUACCAGGGCUUCCAGAGCAUGUACACCUUCGUGAC |
| | CAGCCAUCUGCCACCUGGCUUCAACGAGUACGACUUCGUGCCCGA |
| | GAGCUUCGACCGGGACAAGACAAUCGCCCUGAUCAUGAACAGCAG |
| | CGGCUCUACCGGACUGCCCAAAGGUGUUGCCCUGCCUCAUAGAAC |
| | UGCCUGCGUGCGGUUUAGCCACGCCAGAGAUCCCAUCUUCGGCAA |
| | CCAGAUCAUCCCCGACACAGCCAUCCUGAGCGUGGUGCCUUUUCA |
| | CCACGGCUUCGGCAUGUUCACCACACUGGGCUACCUGAUCUGCGG |
| | CUUCAGAGUGGUGCUGAUGUACCGCUUCGAGGAAGAACUGUUCCU |
| | GCGGAGCCUGCAGGACUACAAGAUCCAGUCUGCUCUGCUGGUGCC |
| | UACUCUGUUCAGCUUCUUUGCCAAGAGCACCCUGAUCGAUAAGUA |
| | CGACCUGAGCAACCUGCACGAGAUCGCCUCUGGCGGAGCCCCUCU |
| | GUCUAAAGAAGUGGGAGAAGCCGUCGCCAAGCGGUUCCAUCUGCC |
| | UGGCAUCAGACAAGGCUAUGGCCUGACCGAGACAACCAGCGCCAU |
| | UCUGAUUACCCCUGAGGGCGACGAUAAGCCUGGCGCUGUGGGAAA |
| | AGUGGUGCCAUUCUUCGAGGCCAAGGUGGUGGAUCUGGACACCGG |
| | CAAAACACUGGGCGUUAACCAGAGGGGCGAGCUGUGUGUUAGAGG |
| | CCCUAUGAUCAUGAGCGGCUACGUGAACAACCCCGAGGCCACAAA |
| | CGCUCUGAUCGACAAGGAUGGAUGGCUGCACAGCGGCGACAUUGC |
| | CUACUGGGACGAAGAUGAGCACUUCUUCAUCGUGGACCGGCUGAA |
| | GUCCCUGAUCAAGUACAAGGGCUACCAGGUGGCCCCAGCCGAGCU |
| | GGAAUCUAUUCUGCUGCAACACCCCAACAUCUUCGAUGCCGGCGU |
| | UGCAGGACUGCCCGAUGAUGAUGCUGGCGAACUGCCAGCUGCUGU |
| | GGUGGUGCUGGAACACGGCAAGACCAUGACCGAGAAAGAAAUCGU |
| | GGACUACGUGGCCAGCCAAGUGACCACCGCCAAGAAACUGAGAGG |
| | CGGCGUGGUGUUUGUGGACGAGGUGCCAAAAGGCCUGACAGGCAA |
| | GCUGGACGCCCGGAAGAUCAGAGAGAUCCUGAUUAAGGCCAAGAA |
| | AGGCGGCAAGAUCGCCGUGUAAUAGCUAACUAGACAAAACACCAA |
| | ACAAACAAGCUGCGAGCCACGGACUU (SEQ ID NO.: 31) |

### Example 5: LNP Formulation

Ionizable lipids, DSPC, cholesterol, and PEG2K-DMG were dissolved in pure ethanol at a 48.5:10:39:2.5 mol % ratio with a total lipid concentration of 10.8 mM. See, e.g., Qiu et al., PNAS 118:e2020401118 (2021). A 0.10-0.20 mg/mL RNA and/or DNA solution was prepared using acidic buffer (pH 4.0-5.0) containing either: barcoded mRNA encoding firefly luciferase; or mRNA encoding firefly luciferase (fLUC) co-formulated with barcoded DNA at a ratio of 1:1 by weight. The nucleotide and lipid solutions were mixed at a 3:1 volume ratio using the NanoAssemblr microfluidic system at a 12 mL/min total flow rate resulting in rapid mixing and self-assembly of LNPs. Formulations were further dialyzed against PBS (pH 7.4) overnight at 4° C., and buffer exchanged into a sucrose-containing Tris-HCl cryoprotectant buffer for subsequent storage at −80° C. The particle size of formulations was measured by dynamic light scattering (DLS) using a Zetasizer Ultra (Malvern Panalytical). RNA encapsulation efficiency was determined by Ribogreen assay using the complementary payload as a standard (ie. RNA only, DNA only, or DNA/RNA mixture).

TABLE 4
Barcoded LNP Formulations

| | Ionizable | | Acidic | | |
|---|---|---|---|---|---|
| Form. | Lipid | Payload (s) | Buffer | Size (nm) | PDI |
| F-1 | Lipid 1 | DNA1:Luc | A | 150.7 | 0.008 |
| F-2 | Lipid 2 | DNA3:Luc | B | 122.2 | 0.044 |
| F-3 | Lipid 3 | DNA2:Luc | B | 116.4 | 0.004 |
| F-4 | Lipid 1 | mRNA4 | A | 167.7 | 0.106 |
| F-5 | Lipid 2 | mRNA6 | B | 105.8 | 0.060 |
| F-6 | Lipid 3 | mRNA5 | B | 102.6 | 0.020 |
| F-7 | Lipid 1 | oRNA8A | A | 145.4 | 0.030 |
| F-8 | Lipid 2 | oRNA10A | B | 105.7 | 0.030 |
| F-9 | Lipid 3 | oRNA1A | B | 103.1 | 0.097 |
| F-10 | Lipid 3 | oRNA2A | B | 105.5 | 0.097 |
| F-11 | Lipid 3 | oRNA3A | B | 104.7 | 0.073 |
| F-12 | Lipid 3 | oRNA4A | B | 103.3 | 0.092 |
| F-13 | Lipid 3 | oRNA5A | B | 104.8 | 0.083 |
| F-14 | Lipid 3 | oRNA6A | B | 114.4 | 0.110 |
| F-15 | Lipid 3 | oRNA8A | B | 112.6 | 0.097 |
| F-16 | Lipid 3 | oRNA9A | B | 100.7 | 0.028 |
| F-17 | Lipid 3 | oRNA10A | B | 107.0 | 0.108 |

Buffer A: 25 mM Sodium Acetate, pH 5.0;
Buffer B: 50 mM Citrate, pH 4.0

### Example 6: In Vivo FLuc Mixed Barcode Delivery and Bioluminescence Measurements

LNP/barcode complexes were prepared as described in Example 5. LNP/Barcode complex formulations were made by mixing different LNP/Barcode complex formulations together in equal parts (unless otherwise noted), to form Study Groups 1 and 2. Study Group 1 was dosed with a complex formulation that included formulations F-1, F-2, F-3, F-4, F-5, F-6, F-7, F-8 and F-16 in equal parts. Study Group 2 was dosed with a complex formulation that included formulations F-9, F-10, F-11, F-12, F-13, F-14, F-15, F-16 and F-17 in equal parts, except for F-10 which was mixed at 1/10^{th} the concentration. BALB/c mice were injected via tail vein with 0.01 mg/kg (per unique barcoded payload, except for oRNA2A in F-10, which was dosed at 0.001 mg/kg) for each of Study Groups 1 and 2 in a total volume of 5 mL/kg. Each Study Group was dosed in 6 mice, and an additional 3 mice were dosed with PBS control. At 6 hr post injection, 3 animals from Study Group 1 were humanely euthanized by CO₂ inhalation and organs including liver, spleen, lung, heart, kidney, brain, and blood were collected and snap frozen using liquid nitrogen. At 24 hr post injection, the remaining 3 animals from Study Groups 1 and the first 3 animals from Study Group 2 were injected with 150 mg/kg D-luciferin intraperitoneally and the whole-body bioluminescence signal was acquired ˜10 minutes after injection of D-luciferin using an IVIS Spectrum In Vivo Imaging System (PerkinElmer). Similarly, at 48 hr post injection, the remaining 3 animals from Study Group 2 were injected with 150 mg/kg D-luciferin intraperitoneally and the whole-body bioluminescence signal was acquired ˜10 minutes after injection of D-luciferin using an IVIS Spectrum In Vivo Imaging System (PerkinElmer). Directly after whole body imaging, animals were immediately euthanized by CO₂ inhalation, and organs including liver, spleen, lung, heart and kidneys were harvested and subjected to bioluminescence imaging (BLI) analysis within 10 minutes of animal sacrifice. Organs were collected after imaging and snap frozen using liquid nitrogen. BLI images were detected in the auto-exposure mode. The BLI signal was quantitated using Living Image 4.7 software (Perkin Elmer) following the manufacturer's instruction. After BLI analysis the weights of the collected organs was measured. All in vivo experiments in this study were performed under the approved animal care guidelines.

### Example 7: Barcode Extraction, Sequencing and Data Analysis

### Tissue Homogenization

Entire organs were harvested from each study animal and snap frozen. Snap frozen tissues are cooled in liquid nitrogen and then homogenized using the GENOMAX® Homogenizer (SPEX EW-41019-48) for 1 minute 45 seconds at 1500 RPM. Powderized tissues are stored in −80° C. freezer until used for barcode extraction.

### DNA Barcode Extraction and Library Prep

DNA barcodes are extracted first by scooping a consistent weight of tissue into 1 mL QUICKEXTRACT™ DNA extraction solution (Lucigen QE09050) and extraction followed the manufacturer's suggested protocol. 1 μL QUICKEXTRACT™ product is added into a 20 μL PCR reaction containing 1 μL 10 μM base forward primer, 1 L 10 μM base reverse primer, 10 μL PHUSION™ Hotstart 2× Master Mix (ThermoFisher Scientific), 1 μL DNA barcode spike-in, and 6 μL H₂O. A thermocycler protocol was used with the following parameters: an initial denaturation of 98° C. for 30 seconds, followed by cycling conditions of 98° C. for 10 seconds, 65° C. for 20 seconds, and 72° C. for 15 seconds for 30 cycles, and a final extension at 72° C. for 5 minutes. PCR product was cleaned using a 1.8× bead cleanup with AMPURE® XP (Beckman Coulter A63881) following manufacturer protocols using the KINGFISHER™ Apex. 2[L Cleanup product was used for a second PCR to add indexes and NGS adapters with the following in each sample mix: 5 μL Unique Dual Index mix (IDT for Illumina), 12.5 μL PHUSION™ Hotstart 2× Master Mix (ThermoFisher Scientific), and 5.5 μL H₂O.

mRNA and oRNA Barcode Extraction and Library Prep

RNA barcodes are extracted first by scooping a consistent weight of tissue into lysis buffer. MAGMAX™ MIRVANA™ total RNA isolation kit was used for RNA barcode extraction on the KINGFISHER™ Apex automated system. ˜1.5 g total RNA product from the MAGMAX™ MIRVANA™ is used as input for reverse transcription, which uses the following master mix: 1 μL 2 μM gene specific reverse transcription primer, 1 μL 10 mM dNTP, 1 μL spike RNA, and variable amounts of H₂O up to 13 μL. Samples were incubated at 65° C. for 5 minutes, then set on ice for 1 minute. 7 μL of RT master mix containing the following reagents were added to each sample: 4 μL 5× SSIV Buffer, 1 μL 100 mM dithiothreitol (DTT), 1 μL RNase inhibitor, and 1 μL SUPERSCRIPT™ IV reverse transcriptase. The plate is incubated at 50° C. for 10 minutes, and then inactivated with an incubation at 80° C. for 10 minutes. 8 μL of reverse transcription product is added into a 25 μL PCR reaction containing 1.25 μL 10 μM base forward primer, 1.25 μL 10 μM base reverse primer, 12.5 μL PHUSION™ Hotstart 2× Master Mix (ThermoFisher Scientific), and 2[L H₂O. A thermocycler protocol was used with the following parameters: an initial denaturation of 98° C. for 30 seconds, followed by cycling conditions of 98° C. for 10 seconds, 57° C. for 20 seconds, and 72° C. for 15 seconds for 30 cycles, and a final extension at 72° C. for 5 minutes. PCR product was cleaned using a 1.8× bead cleanup with AMPURE® XP (Beckman Coulter A63881) following manufacturer protocols using the KINGFISHER™ Apex. Cleanup product (2[L) was used for a second PCR to add indexes and next-generation sequencing (NGS) adapters with the following in each sample mix: 5 μL Unique Dual Index mix (IDT for Illumina), 12.5 μL PHUSION™ Hotstart 2× Master Mix (ThermoFisher Scientific), and 5.5 μL H₂O.

DNA, mRNA, and oRNA Quantification

PCR products from all barcodes are run on the Tapestation (Agilent G2991AA) using the D1000 kit (Agilent 5067-5582) to confirm existence of the desired amplicon. All barcode libraries are then quantified using the QUBIT™ 1× dsDNA Kit (ThermoFisher Q33231) following manufacturer's protocol. At least 250ng of each sample library was used as input for a normalization reaction using the SEQUELPREP™ normalization plate (ThermoFisher A1051001). The libraries were normalized according to manufacturer's instructions and then then quantified using the QUBIT™ 1× dsDNA Kit (ThermoFisher Q33231) following manufacturer's protocol.

### Sequencing

Libraries were diluted to 550 pM in resuspension buffer (RSB) Tween provided with the Illumina sequencing cartridge. A PhiX control was also diluted to 550 pM and added at 30% of the entire final loading library. 20 μL of the library mixed with the PhiX control were loaded onto the sequencing cartridge and the sequencer was loaded and started according to manufacturer's instructions. Libraries were sequenced on the NextSeq2000 (Illumina).

### Data Analysis

Data was analyzed consistently with methods previously described in the literature (e.g. Dahlman, et al. Proc Natl Acad Sci USA. 2017 Feb. 21; 114(8): 2060-2065; Guimaraes, et al. J Control Release. 2019 Dec. 28; 316: 404-417.). Briefly, custom python scripts were written to produce a count of each barcode in each sample. These scripts process sequencing reads by trimming them for quality, merging pairs into single reads, inspecting the reads for presence of expected constant regions of the PCR amplicon, identifying the barcode and UMI, and binning them according to the barcode/UMI combination they harbor. These bins of reads are then quantitated to produce an estimate of the relative abundance of each barcoded species in the samples and then compared to the apparent abundance of the spike-in control. Estimated absolute abundance of each barcoded species is calculated from the sample/control ratio. Abundances are then compared between samples representing distinct tissues to estimate the distribution of the barcoded species across the tested tissues.

### DISCUSSION

Uniquely barcoded DNAs, mRNAs, and oRNAs were individually formulated with three LNPs and co-delivered into a single mouse to compare the relative biodistributions of the three LNPs and to compare the consistencies of biodistribution across DNA, mRNA, and oRNA.

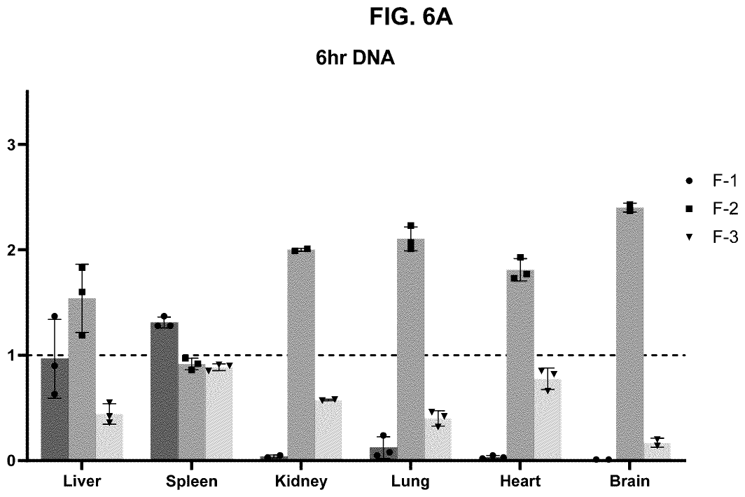

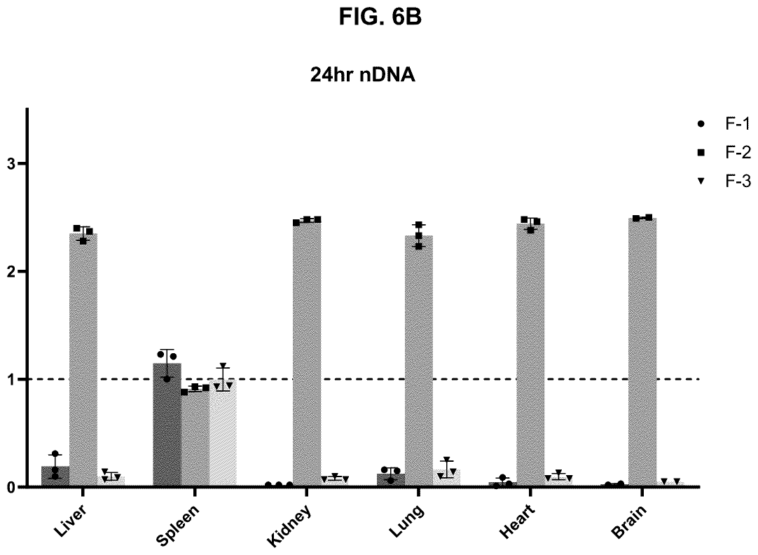

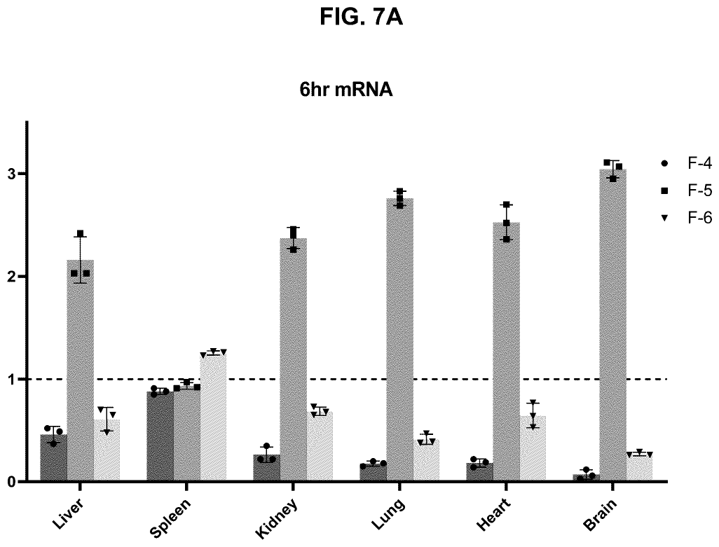

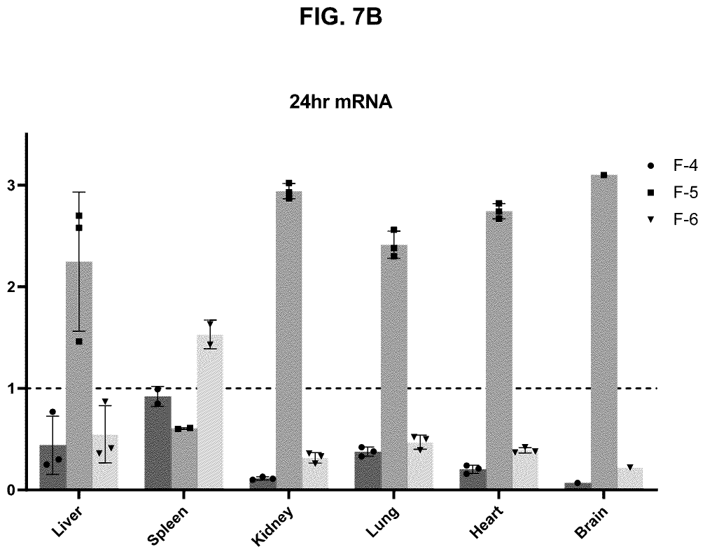

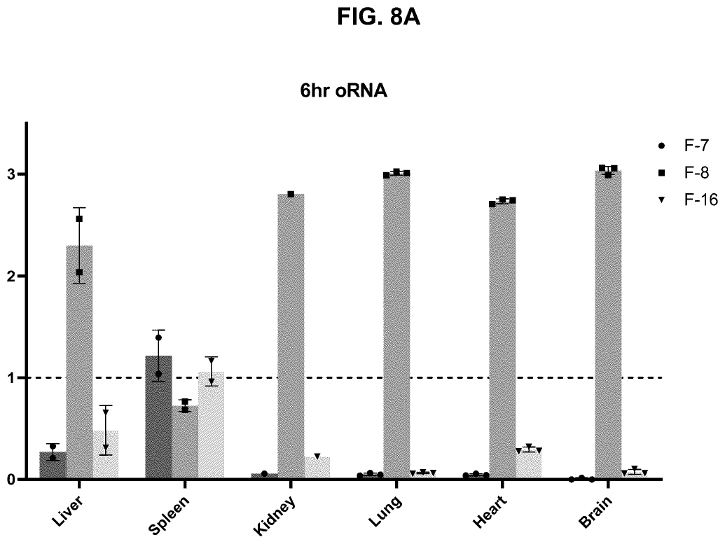

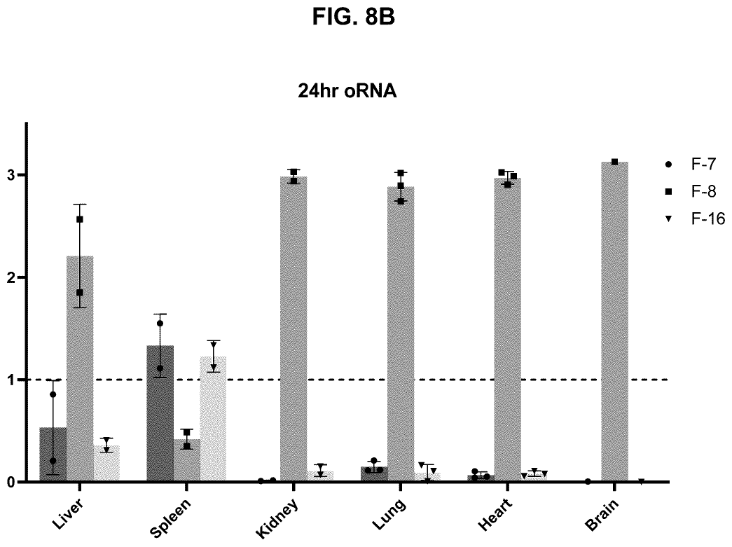

FIGS. 6A, 6B, 7A, 7B, 8A and 8B show that the use of barcoded DNA, mIRNA, and oRNA in combination with various LNPs can be detected using Next Generation Sequencing. The biodistribution of each LNP was able to be determined by the presence of the barcoded cargo. The data also demonstrates that the various barcoded cargos are representative of each other, meaning that the biodistribution of LNPs formulated with barcoded DNAs was consistent with the biodistribution of LNPs when formulated with oRNAs. Specifically, the data show the ratios of individual barcodes within LNPs formulated with Lipids 1, 2 and 3 as detected across liver, spleen, kidney, lung, heart, and brain normalized to the input material. LNPs formulated with Lipid 2 generally showed superior delivery in most organs tested (liver, kidney, lung, heart and brain), as compared to the formulations using Lipids 1 and 3. However, all three LNPs reach the spleen with approximately equivalent efficacy suggesting that all three LNPs demonstrated some level of spleen tropism. Importantly, biodistribution ratios measured utilizing barcoded DNA, mRNA and oRNA were consistent between polynucleotide varieties, demonstrating the viability of utilizing barcoded oRNA for biodistribution studies. Taken together, these data demonstrate the ability of this assay to detect and rank multiple co-delivered LNPs via Next Generation Sequencing and that oRNAs can be successfully used in this platform, with consistent results as compared to both DNA and mRNA.

Without intending to be limited to any particular theory, the consistent results when comparing the barcoded DNA, mRNA and oRNA, in combination with the overall benefits of utilizing oRNA payloads (including but not limited to: lower immunogenicity, increased stability, superior packing efficiency) suggests that oRNAs comprising unique identifier barcodes are an attractive alternative to utilizing DNA and mRNA barcodes for biodistribution studies.

### X. ADDITIONAL EMBODIMENTS

In some embodiments, there is a method for determining the biodistribution of one or more pharmaceutical delivery vehicles upon administration to a subject, the method comprising:
a. formulating the one or more pharmaceutical delivery vehicles to comprise a first engineered polynucleotide comprising:
   i. optionally a payload sequence region comprising a coding nucleic acid sequence or non-coding nucleic acid sequence;
   ii. a unique nucleotide identifier sequence;
   iii. at least one flanking sequence region located at one or more locations selected from upstream of the unique nucleotide identifier sequence and downstream of the unique nucleotide identifier sequence; and
   iv. optionally, at least one regulatory sequence region;
b. administering the one or more pharmaceutical delivery vehicles comprising the first engineered polynucleotide to a subject;
c. collecting at least one sample from the subject at one or more time points after administration of the one or more pharmaceutical delivery vehicles comprising the engineered polynucleotide; and
d. analyzing the at least one sample to determine the amount of the one or more pharmaceutical delivery vehicles present in the at least one sample by measuring the amount of the unique nucleotide identifier sequence present in the sample;
wherein, when more than one distinct pharmaceutical delivery vehicle is administered to the subject, each distinct pharmaceutical delivery vehicle comprises a distinct engineered polynucleotide comprising a different unique nucleotide identifier sequence.

In some embodiments, two or more distinct pharmaceutical delivery vehicles are administered to the subject.

In some embodiments, at least five distinct pharmaceutical delivery vehicles are administered to the subject.

In some embodiments, at least ten distinct pharmaceutical delivery vehicles are administered to the subject.

In some embodiments, the at least one sample collected from the subject is collected from a tissue, an organ or fluid selected from the group consisting of whole blood, plasma, lymph, anal canal, arteries, ascending colon, bladder, bone marrow, brain, bronchi, bronchioles, bulbourethral glands, capillaries, cecum, cerebellum, cerebral hemispheres, cerebrum, cervix, choroid plexus, clitoris, cranial nerves, descending colon, diencephalon, duodenum, ear, enteric nervous system, epididymis, esophagus, external reproductive organs, fallopian tubes, gallbladder, ganglia, gustatory, gut-associated lymphoid tissue, heart, ileum, internal reproductive organs, interstitium, jejunum, joints, kidneys, large intestine, larynx, ligaments, liver, lungs, lymph node, lymphatic vessel, mammary glands, medulla oblongata, mesentery, midbrain, mouth, muscles of breathing, nasal cavity, nerves, olfactory, ovaries, pancreas, parotid glands, penis, pharynx, placenta, pons, prostate, rectum, salivary glands, scrotum, seminal vesicles, sigmoid colon, skeleton, skin, small intestine, spinal nerves, spleen, stomach, subcutaneous tissue, sublingual glands, submandibular glands, teeth, tendons, testes, the brainstem, the spinal cord, the ventricular system, thymus, tongue, tonsils, trachea, transverse colon, ureter, urethra, uterus, vagina, vas deferens, veins, and vulva.

In some embodiments, the one or more pharmaceutical delivery vehicles is administered intravenously, orally, and intramuscularly.

In some embodiments, the first engineered polynucleotide is RNA.

In some embodiments, the first engineered polynucleotide is circular RNA.

In some embodiments, the first engineered polynucleotide is mRNA.

In some embodiments, the first engineered polynucleotide is small RNA.

In some embodiments, the first engineered polynucleotide is miRNA.

In some embodiments, the first engineered polynucleotide is transfer RNA.

In some embodiments, the first engineered polynucleotide is siRNA.

In some embodiments, the first engineered polynucleotide is rRNA.

In some embodiments, the first engineered polynucleotide is DNA.

In some embodiments, the first engineered polynucleotide comprises an internal ribosome entry site (IRES) sequence that is operably linked to the payload sequence region.

In some embodiments, the first engineered polynucleotide comprises a promotor sequence that is operably linked to the payload sequence region.

In some embodiments, the first engineered polynucleotide comprises a termination element comprising at least one stop codon.

In some embodiments, the first engineered polynucleotide comprises a regulatory element.

In some embodiments, the first engineered polynucleotide comprises at least one masking agent.

In some embodiments, the first engineered polynucleotide comprises the payload sequence.

In some embodiments, the payload sequence region comprises a non-coding nucleic acid sequence.

In some embodiments, the payload sequence region comprises a coding nucleic acid sequence.

In some embodiments, the first engineered polynucleotide does not comprise the payload sequence.

In some embodiments, the one or more pharmaceutical delivery vehicles further comprise a second engineered polynucleotide, wherein the second engineered polynucleotide comprises a payload sequence region comprising a coding nucleic acid sequence or non-coding nucleic acid sequence.

In some embodiments, the second engineered polynucleotide is an RNA.

In some embodiments, the second engineered polynucleotide is an mRNA.

In some embodiments, the second engineered polynucleotide is a circular RNA.

In some embodiments, the second engineered polynucleotide is small RNA.

In some embodiments, the second engineered polynucleotide is miRNA.

In some embodiments, the second engineered polynucleotide is transfer RNA.

In some embodiments, the second engineered polynucleotide is siRNA.

In some embodiments, the second engineered polynucleotide is rRNA.

In some embodiments, the second engineered polynucleotide is DNA.

In some embodiments, at least one nucleotide of the first engineered polynucleotide and/or second engineered polynucleotide is chemically modified to comprise at least one non-naturally occurring nucleotide selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine,2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the unique identifier sequence comprises 5 to 10 nucleotides.

In some embodiments, the one or more pharmaceutical delivery vehicles are each independently selected from lipid nanoparticles, non-lipid nanoparticles, exosomes, liposomes, micelles, viral particles, and polymeric nanoparticles.

In some embodiments, the one or more pharmaceutical delivery vehicles are lipid nanoparticles.

In some embodiments, the lipid nanoparticle comprises:
a. one or more ionizable lipids;
b. one or more structural lipids;
c. one or more PEGylated lipids; and
d. one or more phospholipids.

In some embodiments, the one or more ionizable lipids is selected from the group consisting of:
a. the ionizable lipids disclosed in Table 2; and
b. an ionizable lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; and WO 2019/089828A1.

In some embodiments, the one or more structural lipids are selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone.

In some embodiments, the one or more PEGylated lipids are selected from the group consisting of PEG-c-DOMG, PEG-DMG, PEG-DLPE, PEG-DMPE, PEG-DPPC, and PEG-DSPE.

In some embodiments, the one or more phospholipids are selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the lipid nanoparticle comprises about 48.5 mol % of ionizable lipid, about 10 mol % of phospholipid, about 40 mol % of structural lipid, and about 1.5 mol % of PEGylated lipid.

In some embodiments, the lipid nanoparticle comprises about 48.5 mol % of ionizable lipid, about 10 mol % of phospholipid, about 39 mol % of structural lipid, and about 2.5 mol % of PEGylated lipid.

In some embodiments, the lipid nanoparticle further comprises a targeting moiety operably connected to the lipid nanoparticle.

In some embodiments, the lipid nanoparticle further comprises one or more additional components selected from the group consisting of DDAB, EPC, 14PA, 18BMP, DODAP, DOTAP, and C₁₂-200.

In some embodiments, the DNA comprises a polynucleotide selected from SEQ ID NOs. 1-11, or a sequence having at least about 70%, at least about 75%, about at least 80%, about at least 85%, about at least 90%, about at least 91%, about at least 92%, about at least 93%, about at least 94%, about at least 95%, about at least 96%, about at least 97%, about at least 98%, about at least 99%%, about at least 99.1%, about at least 99.2%, about at least 99.3%, about at least 99.4%, about at least 99.5%, about at least 99.6%, about at least 99.7%, about at least 99.8% or about at least 99.9% sequence identity with a sequence selected from SEQ ID NOs. 1-11.

In some embodiments, there is an engineered polynucleotide comprising:
a. a payload sequence region comprising a coding nucleic acid sequence or non-coding nucleic acid sequence;
b. a unique nucleotide identifier sequence that is a barcode sequence;
c. at least one flanking sequence region located at one or more locations selected from upstream of the barcode sequence and downstream of the barcode sequence; and
d. optionally, at least one regulatory sequence region.

In some embodiments, the engineered polynucleotide is DNA.

In some embodiments, the engineered polynucleotide is RNA.

In some embodiments, the engineered polynucleotide is circular RNA.

In some embodiments, the engineered polynucleotide is mRNA.

In some embodiments, the engineered polynucleotide is small RNA.

In some embodiments, the engineered polynucleotide is miRNA.

In some embodiments, the engineered polynucleotide is transfer RNA.

In some embodiments, the engineered polynucleotide is siRNA.

In some embodiments, the engineered polynucleotide is rRNA.

In some embodiments, the RNA inhibits or suppresses the expression of a target of interest in a cell.

In some embodiments, the inhibition or suppression is about 30%, about 40%, about 50%, about 60%, about 70%, about 80%, about 85%, about 90%, about 95%, or about 100%, or at least about 20-30%, about 20-40%, about 20-50%, about 20-60%, about 20-70%, about 20-80%, about 20-90%, about 20-95%, about 20-100%, about 30-40%, about 30-50%, about 30-60%, about 30-70%, about 30-80%, about 30-90%, about 30-95%, about 30-100%, about 40-50%, about 40-60%, about 40-70%, about 40-80%, about 40-90%, about 40-95%, about 40-100%, about 50-60%, about 50-70%, about 50-80%, about 50-90%, about 50-95%, about 50-100%, about 60-70%, about 60-80%, about 60-90%, about 60-95%, about 60-100%, about 70-80%, about 70-90%, about 70-95%, about 70-100%, about 80-90%, about 80-95%, about 80-100%, about 90-95%, about 90-100% or about 95-100%.

In some embodiments, the polynucleotide comprises an internal ribosome entry site (IRES) sequence that is operably linked to the payload sequence region.

In some embodiments, the engineered polynucleotide comprises a promotor sequence that is operably linked to the payload sequence region.

In some embodiments, the polynucleotide comprises a termination element, wherein the termination element comprises at least one stop codon.

In some embodiments, the polynucleotide comprises a regulatory element.

In some embodiments, the polynucleotide comprises at least one masking agent.

In some embodiments, the engineered polynucleotide is produced using in vitro transcription.

In some embodiments, the payload sequence region comprises a non-coding nucleic acid sequence.

In some embodiments, the payload sequence region comprises a coding nucleic acid sequence.

In some embodiments, at least 5%, at least 10%, at least 20%, at least 30%, at least 40%, at least 50%, at least 60%, at least 70%, at least 80%, at least 90%, or at least 100% of the nucleotides are chemically modified nucleotides, wherein the chemically modified nucleotides are non-naturally occurring nucleotides.

In some embodiments, at least one nucleotide of the engineered polynucleotide is chemically modified to comprise at least one non-naturally occurring nucleotide selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine,2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the DNA comprises a polynucleotide selected from SEQ ID NOs. 1-11, or a sequence having at least 70%, at least 75%, at least 80%, at least 85%, at least 90%, at least 91%, at least 92%, at least 93%, at least 94%, at least 95%, at least 96%, at least 97%, at least 98%, at least 99%%, at least 99.1%, at least 99.2%, at least 99.3%, at least 99.4%, at least 99.5%, at least 99.6%, at least 99.7%, at least 99.8% or at least 99.9% sequence identity with a sequence selected from SEQ ID NOs. 1-11.

In some embodiments, there is a pharmaceutical composition comprising:
a. a pharmaceutical delivery vehicle; and
b. an engineered polynucleotide of any one of claims 49-71.

In some embodiments, the one or more pharmaceutical delivery vehicles are lipid nanoparticles.

In some embodiments, the lipid nanoparticle comprises:
a. one or more ionizable lipids;
b. one or more structural lipids;
c. one or more PEGylated lipids; and
d. one or more phospholipids.

In some embodiments, the one or more ionizable lipids is selected from the group consisting of:
a. the ionizable lipids disclosed in Table 2; and
b. an ionizable lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; and WO 2019/089828A1.

In some embodiments, the one or more structural lipids are selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone.

In some embodiments, the one or more PEGylated lipids are selected from the group consisting of PEG-c-DOMG, PEG-DMG, PEG-DLPE, PEG-DMPE, PEG-DPPC, and PEG-DSPE.

In some embodiments, the one or more phospholipids are selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the lipid nanoparticle comprises about 48.5 mol % of ionizable lipid, about 10 mol % of phospholipid, about 40 mol % of structural lipid, and about 1.5 mol % of PEGylated lipid.

In some embodiments, the lipid nanoparticle comprises about 48.5 mol % of ionizable lipid, about 10 mol % of phospholipid, about 39 mol % of structural lipid, and about 2.5 mol % of PEGylated lipid.

In some embodiments, the lipid nanoparticle further comprises a targeting moiety operably connected to the lipid nanoparticle.

In some embodiments, the lipid nanoparticle further comprises one or more additional components selected from the group consisting of DDAB, EPC, 14PA, 18BMP, DODAP, DOTAP, and C₁₂-200.

In some embodiments, there is a method for determining the biodistribution of one or more pharmaceutical delivery vehicles upon administration to a subject, the method comprising:
a. formulating the one or more pharmaceutical delivery vehicles to comprise a first engineered polynucleotide comprising:
i. a unique nucleotide identifier sequence that is a barcode sequence;
ii. at least one flanking sequence region located at one or more locations selected from upstream of the unique identifier sequence and downstream of the barcode sequence; and
iii. optionally, at least one regulatory sequence region;
and a second engineered polynucleotide comprising a payload sequence region comprising a coding nucleic acid sequence or a non-coding nucleic acid sequence;
b. administering the one or more pharmaceutical delivery vehicles comprising the first and second engineered polynucleotides to a subject;
c. collecting at least one sample from the subject at one or more time points after administration of the one or more pharmaceutical delivery vehicles comprising the engineered polynucleotide; and
d. analyzing the at least one sample to determine the amount of the one or more pharmaceutical delivery vehicles present in the at least one sample by measuring the amount of unique identifier sequence(s) present in the sample;
wherein, when more than one distinct pharmaceutical delivery vehicle is administered to the subject, each distinct pharmaceutical delivery vehicle comprises a distinct first engineered polynucleotide comprising a different unique identifier sequence.

In some embodiments, two or more distinct pharmaceutical delivery vehicles are administered to the subject.

In some embodiments, at least five distinct pharmaceutical delivery vehicles are administered to the subject.

In some embodiments, at least ten distinct pharmaceutical delivery vehicles are administered to the subject.

In some embodiments, the at least one sample collected from the subject is collected from a tissue, organ or fluid selected from the group consisting of whole blood, plasma, lymph, anal canal, arteries, ascending colon, bladder, bone marrow, brain, bronchi, bronchioles, bulbourethral glands, capillaries, cecum, cerebellum, cerebral hemispheres, cerebrum, cervix, choroid plexus, clitoris, cranial nerves, descending colon, diencephalon, duodenum, ear, enteric nervous system, epididymis, esophagus, external reproductive organs, fallopian tubes, gallbladder, ganglia, gustatory, gut-associated lymphoid tissue, heart, ileum, internal reproductive organs, interstitium, jejunum, joints, kidneys, large intestine, larynx, ligaments, liver, lungs, lymph node, lymphatic vessel, mammary glands, medulla oblongata, mesentery, midbrain, mouth, muscles of breathing, nasal cavity, nerves, olfactory, ovaries, pancreas, parotid glands, penis, pharynx, placenta, pons, prostate, rectum, salivary glands, scrotum, seminal vesicles, sigmoid colon, skeleton, skin, small intestine, spinal nerves, spleen, stomach, subcutaneous tissue, sublingual glands, submandibular glands, teeth, tendons, testes, the brainstem, the spinal cord, the ventricular system, thymus, tongue, tonsils, trachea, transverse colon, ureter, urethra, uterus, vagina, vas deferens, veins, and vulva.

In some embodiments, the administration of the one or more pharmaceutical delivery vehicles is via a method selected from intravenous, oral, and intramuscular.

In some embodiments, the first engineered polynucleotide is RNA.

In some embodiments, the first engineered polynucleotide is circular RNA.

In some embodiments, the first engineered polynucleotide is mRNA.

In some embodiments, the first engineered polynucleotide is small RNA.

In some embodiments, the first engineered polynucleotide is miRNA.

In some embodiments, the first engineered polynucleotide is transfer RNA.

In some embodiments, the first engineered polynucleotide is siRNA.

In some embodiments, the first engineered polynucleotide is rRNA.

In some embodiments, the first engineered polynucleotide is DNA.

In some embodiments, the first engineered polynucleotide comprises an internal ribosome entry site (IRES) sequence that is operably linked to the payload sequence region.

In some embodiments, the first engineered polynucleotide comprises a promotor sequence that is operably linked to the payload sequence region.

In some embodiments, the first engineered polynucleotide comprises a termination element comprising at least one stop codon.

In some embodiments, the first engineered polynucleotide comprises a regulatory element.

In some embodiments, the first engineered polynucleotide comprises at least one masking agent.

In some embodiments, the second engineered polynucleotide is an RNA.

In some embodiments, the second engineered polynucleotide is an mRNA.

In some embodiments, the second engineered polynucleotide is a circular RNA.

In some embodiments, the second engineered polynucleotide is small RNA.

In some embodiments, the second engineered polynucleotide is miRNA.

In some embodiments, the second engineered polynucleotide is transfer RNA.

In some embodiments, the second engineered polynucleotide is siRNA.

In some embodiments, the second engineered polynucleotide is rRNA.

In some embodiments, the second engineered polynucleotide is DNA.

In some embodiments, at least one nucleotide of the first engineered polynucleotide and/or the second engineered polynucleotide is chemically modified to comprise at least one non-naturally occurring nucleotide selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine,2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the unique identifier sequence comprises 5 to 10 nucleotides.

In some embodiments, the one or more pharmaceutical delivery vehicles are each independently selected from lipid nanoparticles, non-lipid nanoparticles, exosomes, liposomes, micelles, viral particles, and polymeric nanoparticles.

In some embodiments, the one or more pharmaceutical delivery vehicles are lipid nanoparticles.

In some embodiments, the lipid nanoparticle comprises:
a. one or more ionizable lipids;
b. one or more structural lipids;
c. one or more PEGylated lipids; and
d. one or more phospholipids.

In some embodiments, the one or more ionizable lipids is selected from the group consisting of:
a. the ionizable lipids disclosed in Table 2; and
b. an ionizable lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; and WO 2019/089828A1.

In some embodiments, the one or more structural lipids are selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone.

In some embodiments, the one or more PEGylated lipids are selected from the group consisting of PEG-c-DOMG, PEG-DMG, PEG-DLPE, PEG-DMPE, PEG-DPPC, and PEG-DSPE.

In some embodiments, the one or more phospholipids are selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the lipid nanoparticle comprises about 48.5 mol % of ionizable lipid, about 10 mol % of phospholipid, about 40 mol % of structural lipid, and about 1.5 mol % of PEGylated lipid.

In some embodiments, the lipid nanoparticle comprises about 48.5 mol % of ionizable lipid, about 10 mol % of phospholipid, about 39 mol % of structural lipid, and about 2.5 mol % of PEGylated lipid.

In some embodiments, the lipid nanoparticle further comprises a targeting moiety operably connected to the lipid nanoparticle.

In some embodiments, the lipid nanoparticle further comprises one or more additional components selected from the group consisting of DDAB, EPC, 14PA, 18BMP, DODAP, DOTAP, and C₁₂-200.

In some embodiments, the DNA comprises a polynucleotide selected from SEQ ID NOs. 1-11, or a sequence having at least 70%, at least 75%, at least 80%, at least 85%, at least 90%, at least 91%, at least 92%, at least 93%, at least 94%, at least 95%, at least 96%, at least 97%, at least 98%, at least 99%%, at least 99.1%, at least 99.2%, at least 99.3%, at least 99.4%, at least 99.5%, at least 99.6%, at least 99.7%, at least 99.8% or at least 99.9% sequence identity with a sequence selected from SEQ ID NOs. 1-11.

### XI. EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a” and “an” are used herein to refer to one or to more than one (i.e., to at least one) of the grammatical object of the article unless indicated to the contrary or otherwise evident from the context. By way of example, “an element” means one element or more than one element. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A method for determining the biodistribution of one or more pharmaceutical delivery vehicles upon administration to a subject, the method comprising:
a) formulating the one or more pharmaceutical delivery vehicles to comprise a first engineered polynucleotide comprising:
i) optionally a payload sequence region comprising a coding nucleic acid sequence or non-coding nucleic acid sequence;
ii) a unique nucleotide identifier sequence;
iii) at least one flanking sequence region located at one or more locations selected from upstream of the unique nucleotide identifier sequence and downstream of the unique nucleotide identifier sequence; and
iv) optionally, at least one regulatory sequence region;
b) administering the one or more pharmaceutical delivery vehicles comprising the first engineered polynucleotide to a subject;
c) collecting at least one sample from the subject at one or more time points after administration of the one or more pharmaceutical delivery vehicles comprising the engineered polynucleotide; and
d) analyzing the at least one sample to determine the amount of the one or more pharmaceutical delivery vehicles present in the at least one sample by measuring the amount of the unique nucleotide identifier sequence present in the sample;
wherein, when more than one distinct pharmaceutical delivery vehicle is administered to the subject, each distinct pharmaceutical delivery vehicle comprises a distinct engineered polynucleotide comprising a different unique nucleotide identifier sequence.

2. The method of claim 1, wherein two or more distinct pharmaceutical delivery vehicles are administered to the subject.

3. The method of any one of claims 1-2, wherein at least five distinct pharmaceutical delivery vehicles are administered to the subject.

4. The method of any one of claims 1-3, wherein at least ten distinct pharmaceutical delivery vehicles are administered to the subject.

5. The method of any one of claims 1-4, wherein the at least one sample collected from the subject is collected from a tissue, an organ or fluid selected from the group consisting of whole blood, plasma, lymph, anal canal, arteries, ascending colon, bladder, bone marrow, brain, bronchi, bronchioles, bulbourethral glands, capillaries, cecum, cerebellum, cerebral hemispheres, cerebrum, cervix, choroid plexus, clitoris, cranial nerves, descending colon, diencephalon, duodenum, ear, enteric nervous system, epididymis, esophagus, external reproductive organs, fallopian tubes, gallbladder, ganglia, gustatory, gut-associated lymphoid tissue, heart, ileum, internal reproductive organs, interstitium, jejunum, joints, kidneys, large intestine, larynx, ligaments, liver, lungs, lymph node, lymphatic vessel, mammary glands, medulla oblongata, mesentery, midbrain, mouth, muscles of breathing, nasal cavity, nerves, olfactory, ovaries, pancreas, parotid glands, penis, pharynx, placenta, pons, prostate, rectum, salivary glands, scrotum, seminal vesicles, sigmoid colon, skeleton, skin, small intestine, spinal nerves, spleen, stomach, subcutaneous tissue, sublingual glands, submandibular glands, teeth, tendons, testes, the brainstem, the spinal cord, the ventricular system, thymus, tongue, tonsils, trachea, transverse colon, ureter, urethra, uterus, vagina, vas deferens, veins, and vulva.

6. The method of any one of claims 1-5, wherein the one or more pharmaceutical delivery vehicles is administered intravenously, orally, and intramuscularly.

7. The method of any one of claims 1-6, wherein the first engineered polynucleotide is RNA.

8. The method of any one of claims 1-7, wherein the first engineered polynucleotide is circular RNA.

9. The method of any one of claims 1-7, wherein the first engineered polynucleotide is mRNA.

10. The method of any one of claims 1-7, wherein the first engineered polynucleotide is small RNA.

11. The method of any one of claims 1-7, wherein the first engineered polynucleotide is miRNA.

12. The method of any one of claims 1-7, wherein the first engineered polynucleotide is transfer RNA.

13. The method of any one of claims 1-7, wherein the first engineered polynucleotide is siRNA.

14. The method of any one of claims 1-7, wherein the first engineered polynucleotide is rRNA.

15. The method of any one of claims 1-6, wherein the first engineered polynucleotide is DNA.

16. The method of any one of claims 7-9 or 15, wherein the first engineered polynucleotide comprises an internal ribosome entry site (IRES) sequence that is operably linked to the payload sequence region.

17. The method of any one of claims 7-9,15 or 16, wherein the first engineered polynucleotide comprises a promotor sequence that is operably linked to the payload sequence region.

18. The method of any one of claims 7-9 or 15-17, wherein the first engineered polynucleotide comprises a termination element comprising at least one stop codon.

19. The method of any of claims 7-11, 13, or 15-18, wherein the first engineered polynucleotide comprises a regulatory element.

20. The method of any of claims 7-11, 13, or 15, wherein the first engineered polynucleotide comprises at least one masking agent.

21. The method of any one of claims 1-20, wherein the first engineered polynucleotide comprises the payload sequence.

22. The method of claim 21, wherein the payload sequence region comprises a non-coding nucleic acid sequence.

23. The method of claim 21, wherein the payload sequence region comprises a coding nucleic acid sequence.

24. The method of any one of claims 1-20, wherein the first engineered polynucleotide does not comprise the payload sequence.

25. The method of any one of claim 1-24, wherein the one or more pharmaceutical delivery vehicles further comprise a second engineered polynucleotide, wherein the second engineered polynucleotide comprises a payload sequence region comprising a coding nucleic acid sequence or non-coding nucleic acid sequence.

26. The method of claim 25, wherein the second engineered polynucleotide is an RNA.

27. The method of claim 25, wherein the second engineered polynucleotide is an mRNA.

28. The method of claim 25, wherein the second engineered polynucleotide is a circular RNA.

29. The method of claim 25, wherein the second engineered polynucleotide is small RNA.

30. The method of claim 25, wherein the second engineered polynucleotide is miRNA.

31. The method of claim 25, wherein the second engineered polynucleotide is transfer RNA.

32. The method of claim 25, wherein the second engineered polynucleotide is siRNA.

33. The method of claim 25, wherein the second engineered polynucleotide is rRNA.

34. The method of claim 25, wherein the second engineered polynucleotide is DNA.

35. The method of any of claims 1-34, wherein at least one nucleotide of the first engineered polynucleotide and/or second engineered polynucleotide is chemically modified to comprise at least one non-naturally occurring nucleotide selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine,2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

36. The method of any one of claims 1-35, wherein the unique identifier sequence comprises 5 to 10 nucleotides.

37. The method of any one of claims 1-36, wherein the one or more pharmaceutical delivery vehicles are each independently selected from lipid nanoparticles, non-lipid nanoparticles, exosomes, liposomes, micelles, viral particles, and polymeric nanoparticles.

38. The method of claim 37, wherein the one or more pharmaceutical delivery vehicles are lipid nanoparticles.

39. The method of claim 38, wherein the lipid nanoparticle comprises:
a) one or more ionizable lipids;
b) one or more structural lipids;
c) one or more PEGylated lipids; and
d) one or more phospholipids.

40. The method of claim 39, wherein the one or more ionizable lipids is selected from the group consisting of:
a) the ionizable lipids disclosed in Table 2; and
b) an ionizable lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; and WO 2019/089828A1.

41. The method of any one of claims 39-40, wherein the one or more structural lipids are selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone.

42. The method of any one of claims 39-41, wherein the one or more PEGylated lipids are selected from the group consisting of PEG-c-DOMG, PEG-DMG, PEG-DLPE, PEG-DMPE, PEG-DPPC, and PEG-DSPE.

43. The method of any one of claims 39-42, wherein the one or more phospholipids are selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

44. The method of any one of claims 38-43, wherein the lipid nanoparticle comprises about 48.5 mol % of ionizable lipid, about 10 mol % of phospholipid, about 40 mol % of structural lipid, and about 1.5 mol % of PEGylated lipid.

45. The method of any one of claims 38-44, wherein the lipid nanoparticle comprises about 48.5 mol % of ionizable lipid, about 10 mol % of phospholipid, about 39 mol % of structural lipid, and about 2.5 mol % of PEGylated lipid.

46. The method of any one of claims 38-45, wherein the lipid nanoparticle further comprises a targeting moiety operably connected to the lipid nanoparticle.

47. The method of any one of claims 38-46, wherein the lipid nanoparticle further comprises one or more additional components selected from the group consisting of DDAB, EPC, 14PA, 18BMP, DODAP, DOTAP, and C12-200.

48. The method of claim 15, wherein the DNA comprises a polynucleotide selected from SEQ ID NOs. 1-11, or a sequence having at least about 70%, at least about 75%, about at least 80%, about at least 85%, about at least 90%, about at least 91%, about at least 92%, about at least 93%, about at least 94%, about at least 95%, about at least 96%, about at least 97%, about at least 98%, about at least 99%%, about at least 99.1%, about at least 99.2%, about at least 99.3%, about at least 99.4%, about at least 99.5%, about at least 99.6%, about at least 99.7%, about at least 99.8% or about at least 99.9% sequence identity with a sequence selected from SEQ ID NOs. 1-11.

49. An engineered polynucleotide comprising:
a) a payload sequence region comprising a coding nucleic acid sequence or non-coding nucleic acid sequence;
b) a unique nucleotide identifier sequence that is a barcode sequence;
c) at least one flanking sequence region located at one or more locations selected from upstream of the barcode sequence and downstream of the barcode sequence; and
d) optionally, at least one regulatory sequence region.

50. The engineered polynucleotide of claim 49, wherein the engineered polynucleotide is DNA.

51. The engineered polynucleotide of claim 49, wherein the engineered polynucleotide is RNA.

52. The engineered polynucleotide of claim 49, wherein the engineered polynucleotide is circular RNA.

53. The engineered polynucleotide of claim 49, wherein the engineered polynucleotide is mRNA.

54. The engineered polynucleotide of claim 49, wherein the engineered polynucleotide is small RNA.

55. The engineered polynucleotide of claim 49, wherein the engineered polynucleotide is miRNA.

56. The engineered polynucleotide of claim 49, wherein the engineered polynucleotide is transfer RNA.

57. The engineered polynucleotide of claim 49, wherein the engineered polynucleotide is siRNA.

58. The engineered polynucleotide of claim 49, wherein the engineered polynucleotide is rRNA.

59. The engineered polynucleotide of any of claims 51-58, wherein the RNA inhibits or suppresses the expression of a target of interest in a cell.

60. The engineered polynucleotide of claim 59, wherein the inhibition or suppression is about 30%, about 40%, about 50%, about 60%, about 70%, about 80%, about 85%, about 90%, about 95%, or about 100%, or at least about 20-30%, about 20-40%, about 20-50%, about 20-60%, about 20-70%, about 20-80%, about 20-90%, about 20-95%, about 20-100%, about 30-40%, about 30-50%, about 30-60%, about 30-70%, about 30-80%, about 30-90%, about 30-95%, about 30-100%, about 40-50%, about 40-60%, about 40-70%, about 40-80%, about 40-90%, about 40-95%, about 40-100%, about 50-60%, about 50-70%, about 50-80%, about 50-90%, about 50-95%, about 50-100%, about 60-70%, about 60-80%, about 60-90%, about 60-95%, about 60-100%, about 70-80%, about 70-90%, about 70-95%, about 70-100%, about 80-90%, about 80-95%, about 80-100%, about 90-95%, about 90-100% or about 95-100%.

61. The engineered polynucleotide of any of claims 50-53, wherein the polynucleotide comprises an internal ribosome entry site (IRES) sequence that is operably linked to the payload sequence region.

62. The engineered polynucleotide of any one of claims 50-53 or 61, wherein the engineered polynucleotide comprises a promotor sequence that is operably linked to the payload sequence region.

63. The engineered polynucleotide of any of claims 50-53, 61 or 62 wherein the polynucleotide comprises a termination element, wherein the termination element comprises at least one stop codon.

64. The engineered polynucleotide of any of claims 50-55 or 57, wherein the polynucleotide comprises a regulatory element.

65. The engineered polynucleotide of any of claims 50-55 or 57, wherein the polynucleotide comprises at least one masking agent.

66. The engineered polynucleotide of any of claims 51-59, wherein the engineered polynucleotide is produced using in vitro transcription.

67. The engineered polynucleotide of any of claims 49-66, wherein the payload sequence region comprises a non-coding nucleic acid sequence.

68. The engineered polynucleotide of any of claims 49-53, wherein the payload sequence region comprises a coding nucleic acid sequence.

69. The engineered polynucleotide of any of claims 49-68, wherein at least 5%, at least 10%, at least 20%, at least 30%, at least 40%, at least 50%, at least 60%, at least 70%, at least 80%, at least 90%, or at least 100% of the nucleotides are chemically modified nucleotides, wherein the chemically modified nucleotides are non-naturally occurring nucleotides.

70. The engineered polynucleotide of any of claims 49-69, wherein at least one nucleotide of the engineered polynucleotide is chemically modified to comprise at least one non-naturally occurring nucleotide selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine,2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

71. The engineered polynucleotide of claim 50, wherein the DNA comprises a polynucleotide selected from SEQ ID NOs. 1-11, or a sequence having at least 70%, at least 75%, at least 80%, at least 85%, at least 90%, at least 91%, at least 92%, at least 93%, at least 94%, at least 95%, at least 96%, at least 97%, at least 98%, at least 99%%, at least 99.1%, at least 99.2%, at least 99.3%, at least 99.4%, at least 99.5%, at least 99.6%, at least 99.7%, at least 99.8% or at least 99.9% sequence identity with a sequence selected from SEQ ID NOs. 1-11.

72. A pharmaceutical composition comprising:
a) a pharmaceutical delivery vehicle; and
b) an engineered polynucleotide of any one of claims 49-71.

73. The pharmaceutical composition of claim 72, wherein the one or more pharmaceutical delivery vehicles are lipid nanoparticles.

74. The pharmaceutical composition of claim 73, wherein the lipid nanoparticle comprises:
a) one or more ionizable lipids;
b) one or more structural lipids;
c) one or more PEGylated lipids; and
d) one or more phospholipids.

75. The pharmaceutical composition of claim 74, wherein the one or more ionizable lipids is selected from the group consisting of:
a) the ionizable lipids disclosed in Table 2; and
b) an ionizable lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; and WO 2019/089828A1.

76. The pharmaceutical composition of any one of claims 74-75, wherein the one or more structural lipids are selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone.

77. The pharmaceutical composition of any one of claims 74-76, wherein the one or more PEGylated lipids are selected from the group consisting of PEG-c-DOMG, PEG-DMG, PEG-DLPE, PEG-DMPE, PEG-DPPC, and PEG-DSPE.

78. The pharmaceutical composition of any one of claims 74-77, wherein the one or more phospholipids are selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

79. The pharmaceutical composition of any one of claims 73-78, wherein the lipid nanoparticle comprises about 48.5 mol % of ionizable lipid, about 10 mol % of phospholipid, about 40 mol % of structural lipid, and about 1.5 mol % of PEGylated lipid.

80. The pharmaceutical composition of any one of claims 73-79, wherein the lipid nanoparticle comprises about 48.5 mol % of ionizable lipid, about 10 mol % of phospholipid, about 39 mol % of structural lipid, and about 2.5 mol % of PEGylated lipid.

81. The pharmaceutical composition of any one of claims 73-80, wherein the lipid nanoparticle further comprises a targeting moiety operably connected to the lipid nanoparticle.

82. The pharmaceutical composition of any one of claims 73-81, wherein the lipid nanoparticle further comprises one or more additional components selected from the group consisting of DDAB, EPC, 14PA, 18BMP, DODAP, DOTAP, and C₁₂-200.

83. A method for determining the biodistribution of one or more pharmaceutical delivery vehicles upon administration to a subject, the method comprising:
a) formulating the one or more pharmaceutical delivery vehicles to comprise a first engineered polynucleotide comprising:
i) a unique nucleotide identifier sequence that is a barcode sequence;
ii) at least one flanking sequence region located at one or more locations selected from upstream of the unique identifier sequence and downstream of the barcode sequence; and
iii) optionally, at least one regulatory sequence region;
and a second engineered polynucleotide comprising a payload sequence region comprising a coding nucleic acid sequence or a non-coding nucleic acid sequence;
b) administering the one or more pharmaceutical delivery vehicles comprising the first and second engineered polynucleotides to a subject;
c) collecting at least one sample from the subject at one or more time points after administration of the one or more pharmaceutical delivery vehicles comprising the engineered polynucleotide; and
d) analyzing the at least one sample to determine the amount of the one or more pharmaceutical delivery vehicles present in the at least one sample by measuring the amount of unique identifier sequence(s) present in the sample;
wherein, when more than one distinct pharmaceutical delivery vehicle is administered to the subject, each distinct pharmaceutical delivery vehicle comprises a distinct first engineered polynucleotide comprising a different unique identifier sequence.

84. The method of claim 83, wherein two or more distinct pharmaceutical delivery vehicles are administered to the subject.

85. The method of any one of claims 83-84, wherein at least five distinct pharmaceutical delivery vehicles are administered to the subject.

86. The method of any one of claims 83-85, wherein at least ten distinct pharmaceutical delivery vehicles are administered to the subject.

87. The method of any one of claims 83-86, wherein the at least one sample collected from the subject is collected from a tissue, organ or fluid selected from the group consisting of whole blood, plasma, lymph, anal canal, arteries, ascending colon, bladder, bone marrow, brain, bronchi, bronchioles, bulbourethral glands, capillaries, cecum, cerebellum, cerebral hemispheres, cerebrum, cervix, choroid plexus, clitoris, cranial nerves, descending colon, diencephalon, duodenum, ear, enteric nervous system, epididymis, esophagus, external reproductive organs, fallopian tubes, gallbladder, ganglia, gustatory, gut-associated lymphoid tissue, heart, ileum, internal reproductive organs, interstitium, jejunum, joints, kidneys, large intestine, larynx, ligaments, liver, lungs, lymph node, lymphatic vessel, mammary glands, medulla oblongata, mesentery, midbrain, mouth, muscles of breathing, nasal cavity, nerves, olfactory, ovaries, pancreas, parotid glands, penis, pharynx, placenta, pons, prostate, rectum, salivary glands, scrotum, seminal vesicles, sigmoid colon, skeleton, skin, small intestine, spinal nerves, spleen, stomach, subcutaneous tissue, sublingual glands, submandibular glands, teeth, tendons, testes, the brainstem, the spinal cord, the ventricular system, thymus, tongue, tonsils, trachea, transverse colon, ureter, urethra, uterus, vagina, vas deferens, veins, and vulva.

88. The method of any one of claims 83-87, wherein the administration of the one or more pharmaceutical delivery vehicles is via a method selected from intravenous, oral, and intramuscular.

89. The method of any one of claims 83-88, wherein the first engineered polynucleotide is RNA.

90. The method of any one of claims 83-88, wherein the first engineered polynucleotide is circular RNA.

91. The method of any one of claims 83-88, wherein the first engineered polynucleotide is mRNA.

92. The method of any one of claims 83-88, wherein the first engineered polynucleotide is small RNA.

93. The method of any one of claims 83-88, wherein the first engineered polynucleotide is miRNA.

94. The method of any one of claims 83-88, wherein the first engineered polynucleotide is transfer RNA.

95. The method of any one of claims 83-88, wherein the first engineered polynucleotide is siRNA.

96. The method of any one of claims 83-88, wherein the first engineered polynucleotide is rRNA.

97. The method of any one of claims 83-88, wherein the first engineered polynucleotide is DNA.

98. The method of any one of claims 89-91 or 97, wherein the first engineered polynucleotide comprises an internal ribosome entry site (IRES) sequence that is operably linked to the payload sequence region.

99. The method of any one of claims 89-91 or 97, wherein the first engineered polynucleotide comprises a promotor sequence that is operably linked to the payload sequence region.

100. The method of any one of claims 89-91 or 97, wherein the first engineered polynucleotide comprises a termination element comprising at least one stop codon.

101. The method of any of claims 89-93, 95, or 97, wherein the first engineered polynucleotide comprises a regulatory element.

102. The method of any of claims 89-93, 95, or 97, wherein the first engineered polynucleotide comprises at least one masking agent.

103. The method of claim 83, wherein the second engineered polynucleotide is an RNA.

104. The method of claim 83, wherein the second engineered polynucleotide is an mRNA.

105. The method of claim 83, wherein the second engineered polynucleotide is a circular RNA.

106. The method of claim 83, wherein the second engineered polynucleotide is small RNA.

107. The method of claim 83, wherein the second engineered polynucleotide is miRNA.

108. The method of claim 83, wherein the second engineered polynucleotide is transfer RNA.

109. The method of claim 83, wherein the second engineered polynucleotide is siRNA.

110. The method of claim 83, wherein the second engineered polynucleotide is rRNA.

111. The method of claim 83, wherein the second engineered polynucleotide is DNA.

112. The method of any of claims 83-111, wherein at least one nucleotide of the first engineered polynucleotide and/or the second engineered polynucleotide is chemically modified to comprise at least one non-naturally occurring nucleotide selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine,2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

113. The method of any one of claims 83-112, wherein the unique identifier sequence comprises 5 to 10 nucleotides.

114. The method of any one of claims 83-113, wherein the one or more pharmaceutical delivery vehicles are each independently selected from lipid nanoparticles, non-lipid nanoparticles, exosomes, liposomes, micelles, viral particles, and polymeric nanoparticles.

115. The method of claim 114, wherein the one or more pharmaceutical delivery vehicles are lipid nanoparticles.

116. The method of claim 115, wherein the lipid nanoparticle comprises:
a) one or more ionizable lipids;
b) one or more structural lipids;
c) one or more PEGylated lipids; and
d) one or more phospholipids.

117. The method of claim 116, wherein the one or more ionizable lipids is selected from the group consisting of:
a) the ionizable lipids disclosed in Table 2; and
b) an ionizable lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; and WO 2019/089828A1.

118. The method of claim 116, wherein the one or more structural lipids are selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone.

119. The method of claim 116, wherein the one or more PEGylated lipids are selected from the group consisting of PEG-c-DOMG, PEG-DMG, PEG-DLPE, PEG-DMPE, PEG-DPPC, and PEG-DSPE.

120. The method of claim 116, wherein the one or more phospholipids are selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

121. The method of any one of claims 115-120, wherein the lipid nanoparticle comprises about 48.5 mol % of ionizable lipid, about 10 mol % of phospholipid, about 40 mol % of structural lipid, and about 1.5 mol % of PEGylated lipid.

122. The method of any one of claims 115-121, wherein the lipid nanoparticle comprises about 48.5 mol % of ionizable lipid, about 10 mol % of phospholipid, about 39 mol % of structural lipid, and about 2.5 mol % of PEGylated lipid.

123. The method of any one of claims 115-122, wherein the lipid nanoparticle further comprises a targeting moiety operably connected to the lipid nanoparticle.

124. The method of any one of claims 115-123, wherein the lipid nanoparticle further comprises one or more additional components selected from the group consisting of DDAB, EPC, 14PA, 18BMP, DODAP, DOTAP, and C₁₂-200.

125. The method of claim 97, wherein the DNA comprises a polynucleotide selected from SEQ ID NOs. 1-11, or a sequence having at least 70%, at least 75%, at least 80%, at least 85%, at least 90%, at least 91%, at least 92%, at least 93%, at least 94%, at least 95%, at least 96%, at least 97%, at least 98%, at least 99%%, at least 99.1%, at least 99.2%, at least 99.3%, at least 99.4%, at least 99.5%, at least 99.6%, at least 99.7%, at least 99.8% or at least 99.9% sequence identity with a sequence selected from SEQ ID NOs. 1-11.
